(19) United States
(12) Patent Application Publication
(10) Pub. No.: US 2024/0424130 A1
(43) Pub. Date: Dec. 26, 2024

(54) COMPOSITIONS AND METHODS FOR MODULATING DELTA GAMMA CHAIN MEDIATED IMMUNITY

(71) Applicant: JANSSEN BIOTECH, INC., HORSHAM, PA (US)

(72) Inventors: RAJKUMAR GANESAN, BLUE BELL, PA (US); IQBAL S. GREWAL, NEWTOWN, PA (US); SANJAYA SINGH, BLUE BELL, PA (US); MICHAEL RIIS HANSEN, BROOMALL, PA (US)

(73) Assignee: JANSSEN BIOTECH, INC., HORSHAM, PA (US)

(21) Appl. No.: 18/812,337

(22) Filed: Aug. 22, 2024

Related U.S. Application Data
(62) Division of application No. 17/512,195, filed on Oct. 27, 2021, now Pat. No. 12,076,413.
(60) Provisional application No. 63/106,703, filed on Oct. 28, 2020.

(51) Int. Cl. A61K 47/68; A61K 39/00; C07K 16/28

(52) U.S. Cl. CPC A61K 47/6897; C07K 16/2809; A61K 39/00; C07K 2317/24; C07K 2317/31; C07K 2317/35; C07K 2317/56; C07K 2317/565; C07K 2317/73

(57) ABSTRACT

Provided herein, in certain aspects, are antibodies that bind to T cell receptor (TCR) Vγ9 (TRGV9), TCR Vδ2 (TRDV2), or the TCR gamma/delta constant region (TRGDC), as well as recombinant cells containing the vectors, and compositions comprising the antibodies. Methods of making and using the antibodies are also provided.

### CROSS-REFERENCE TO RELATED APPLICATIONS

This application is a divisional application of and claims the benefit of U.S. Ser. No. 17/512,195, filed Oct. 27, 2021, which claims the benefit of U.S. Ser. No. 63/106,703 filed Oct. 28, 2020; the disclosures of which are incorporated by reference herein in their entirety.

### FIELD

Provided herein, in certain aspects, are antibodies that bind to T cell receptor (TCR) Vγ9 (TRGV9), TCR Vδ2 (TRDV2), or the TCR gamma/delta constant region (TRGDC), as well as recombinant cells containing the vectors, and compositions comprising the antibodies. Methods of making and using the antibodies are also provided.

### REFERENCE TO SEQUENCE LISTING SUBMITTED ELECTRONICALLY

This application contains a sequence listing, which is submitted electronically as a XML formatted sequence listing with a file name of “JBI6395USDIV1_SL.xml” and a creation date of Aug. 21, 2024 and having a size of 5,799,673 bytes. The sequence listing submitted is part of the specification and is herein incorporated by reference in its entirety.

### SUMMARY

In one aspect, provided herein is an antibody that binds TRGV9. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:168. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:201; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:202. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:269; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:304. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:337; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:338. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:440. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:609; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:610. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:644. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:712. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:847; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:848. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:881; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:882. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:915; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:916. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:949; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:950. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:983; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:984. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1017; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1018. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1051; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1052. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1085; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1086. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1119; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1120. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1153; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1154. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1187; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1188. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1221; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1222. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1255; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1256. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1289; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1290. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1323; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1324. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1357; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1358. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1391; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1392. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1425; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1426. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1459; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1460. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1493; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1494. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1527; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1528. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1561; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1562. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1595; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1596. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1629; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1630. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1663; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1664. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1697; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1698. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1731; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1732. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1765; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1766. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1799; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1800. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1833; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1834. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1867; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1868. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1901; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1902. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1935; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1936. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1969; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1970. In one embodiment, the TRGV9 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2003; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2004.

In some embodiments, the VH CDR1, VH CDR2, VH CDR3, VL CDR1, VL CDR2, and VL CDR3 amino acid sequences of the TRGV9 antibody are according to the Kabat numbering system. In some embodiments, the VH CDR1, VH CDR2, VH CDR3, VL CDR1, VL CDR2, and VL CDR3 amino acid sequences of the TRGV9 antibody are according to the Chothia numbering system. In some embodiments, the VH CDR1, VH CDR2, VH CDR3, VL CDR1, VL CDR2, and VL CDR3 amino acid sequences of the TRGV9 antibody are according to the AbM numbering system. In some embodiments, the VH CDR1, VH CDR2, VH CDR3, VL CDR1, VL CDR2, and VL CDR3 amino acid sequences of the TRGV9 antibody are according to the Contact numbering system. In some embodiments, the VH CDR1, VH CDR2, VH CDR3, VL CDR1, VL CDR2, and VL CDR3 amino acid sequences of the TRGV9 antibody are according to the IMGT numbering system.

In some embodiments, the antibody binds a TRGV9 antigen. In some embodiments, antibody binds a TRGV9 epitope. In some embodiments, the antibody specifically binds to TRGV9. In some embodiments, the VH CDR1, VH CDR2, VH CDR3, VL CDR1, VL CDR2 and VL CDR3 form a binding site for an antigen of the TRGV9. In some embodiments, the VH CDR1, VH CDR2, VH CDR3, VL CDR1, VL CDR2 and VL CDR3 form a binding site for an epitope of the TRGV9. In some embodiments, the TRGV9 is present on the surface of a T cell.

In some embodiments, the TRGV9 antibody is a humanized antibody. In some embodiments, the antibody is an IgG antibody. In some embodiments, the TRGV9 antibody is an IgG1 antibody. In some embodiments, the TRGV9 antibody is an IgG2 antibody. In some embodiments, the TRGV9 antibody is an IgG3 antibody. In some embodiments, the TRGV9 antibody is an IgG4 antibody. In some embodiments, the TRGV9 antibody comprises a kappa light chain. In some embodiments, the TRGV9 the antibody comprises a lambda light chain. In some embodiments, the TRGV9 antibody is a monoclonal antibody. In some embodiments, the TRGV9 antibody is multivalent. In some embodiments, the TRGV9 antibody is capable of binding at least three antigens. In some embodiments, the TRGV9 antibody is capable of binding at least five antigens. In some embodiments, the TRGV9 antibody is a multispecific antibody. In some embodiments, the TRGV9 antibody is a bispecific antibody.

In another aspect, provided is a nucleic acid sequence encoding a TRGV9 antibody provided herein. Also provided is a vector comprising a nucleic acid sequence encoding a TRGV9 antibody provided herein. Also provided is a host cell comprising a vector comprising a nucleic acid sequence encoding a TRGV9 antibody provided herein. Also provided is a kit comprising vector comprising a nucleic acid sequence encoding a TRGV9 antibody provided herein, and packaging for the same.

In another aspect, provided is a kit comprising a TRGV9 antibody provided herein, and packaging for the same.

In another aspect, provided is a pharmaceutical composition comprising a TRGV9 antibody provided herein, and a pharmaceutically acceptable carrier.

In another aspect, provided is a method of producing a pharmaceutical composition comprising a TRGV9 antibody provided herein, comprising combining the TRGV9 antibody with a pharmaceutically acceptable carrier to obtain the pharmaceutical composition.

In another aspect, provided is a method of activating a T cell expressing TRGV9, comprising contacting the T cell with a TRGV9 antibody provided herein. In certain embodiments, the contacting results in an increase in CD69, CD25, and/or Granzyme B expression, as compared to a control T cell expressing TRGV9.

In another aspect, provided herein is an antibody that binds TRDV2. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2037; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2038. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2071; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2072. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2105; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2106. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2139; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2140. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2173; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2174. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2207; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2208. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2241; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2242. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2275; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2276. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2309; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2310. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2343; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2344. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2377; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2378. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2411; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2412. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2445; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2446. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2479; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2480. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2513; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2514. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2547; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2548. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2581; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2582. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2615; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2616. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2649; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2650. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2683; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2684. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2717; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2718. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2751; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2752. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2785; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2786. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2819; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2820. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2853; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2854. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2887; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2888. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2921; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2922. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2955; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2956. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2989; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2990. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3023; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3024. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3057; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3058. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3091; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3092. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3125; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3126. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3159; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3160. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3193; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3194. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3227; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3228. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3261; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3262. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3295; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3296. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3329; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3330. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3363; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3364. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3397; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3398. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3431; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3432. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3465; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3466. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3499; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3500. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3533; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3534. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3567; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3568. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3601; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3602. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3635; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3636. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3669; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3670. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3703; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3704. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3737; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3738. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3771; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3772. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3805; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3806. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3839; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3840. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3873; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3874. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3907; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3908. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3941; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3942. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3975; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3976. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4009; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4010. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4043; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4044. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4077; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4078. In one embodiment, the TRDV2 antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4111; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4112.

In some embodiments, the VH CDR1, VH CDR2, VH CDR3, VL CDR1, VL CDR2, and VL CDR3 amino acid sequences of the TRDV2 antibody are according to the Kabat numbering system. In some embodiments, the VH CDR1, VH CDR2, VH CDR3, VL CDR1, VL CDR2, and VL CDR3 amino acid sequences of the TRDV2 antibody are according to the Chothia numbering system. In some embodiments, the VH CDR1, VH CDR2, VH CDR3, VL CDR1, VL CDR2, and VL CDR3 amino acid sequences of the TRDV2 antibody are according to the AbM numbering system. In some embodiments, the VH CDR1, VH CDR2, VH CDR3, VL CDR1, VL CDR2, and VL CDR3 amino acid sequences of the TRDV2 antibody are according to the Contact numbering system. In some embodiments, the VH CDR1, VH CDR2, VH CDR3, VL CDR1, VL CDR2, and VL CDR3 amino acid sequences of the TRDV2 antibody are according to the IMGT numbering system.

In some embodiments, the antibody binds a TRDV2 antigen. In some embodiments, antibody binds a TRDV2 epitope. In some embodiments, the antibody specifically binds to TRDV2. In some embodiments, the VH CDR1, VH CDR2, VH CDR3, VL CDR1, VL CDR2 and VL CDR3 form a binding site for an antigen of the TRDV2. In some embodiments, the VH CDR1, VH CDR2, VH CDR3, VL CDR1, VL CDR2 and VL CDR3 form a binding site for an epitope of the TRDV2. In some embodiments, the TRDV2 is present on the surface of a T cell.

In some embodiments, the TRDV2 antibody is a humanized antibody. In some embodiments, the antibody is an IgG antibody. In some embodiments, the TRDV2 antibody is an IgG1 antibody. In some embodiments, the TRDV2 antibody is an IgG2 antibody. In some embodiments, the TRDV2 antibody is an IgG3 antibody. In some embodiments, the TRDV2 antibody is an IgG4 antibody. In some embodiments, the TRDV2 antibody comprises a kappa light chain. In some embodiments, the TRDV2 the antibody comprises a lambda light chain. In some embodiments, the TRDV2 antibody is a monoclonal antibody. In some embodiments, the TRDV2 antibody is multivalent. In some embodiments, the TRDV2 antibody is capable of binding at least three antigens. In some embodiments, the TRDV2 antibody is capable of binding at least five antigens. In some embodiments, the TRDV2 antibody is a multispecific antibody. In some embodiments, the TRDV2 antibody is a bispecific antibody.

In another aspect, provided is a nucleic acid sequence encoding a TRDV2 antibody provided herein. Also provided is a vector comprising a nucleic acid sequence encoding a TRDV2 antibody provided herein. Also provided is a host cell comprising a vector comprising a nucleic acid sequence encoding a TRDV2 antibody provided herein. Also provided is a kit comprising vector comprising a nucleic acid sequence encoding a TRDV2 antibody provided herein, and packaging for the same.

In another aspect, provided is a kit comprising a TRDV2 antibody provided herein, and packaging for the same.

In another aspect, provided is a pharmaceutical composition comprising a TRDV2 antibody provided herein, and a pharmaceutically acceptable carrier.

In another aspect, provided is a method of producing a pharmaceutical composition comprising a TRDV2 antibody provided herein, comprising combining the TRDV2 antibody with a pharmaceutically acceptable carrier to obtain the pharmaceutical composition.

In another aspect, provided is a method of activating a T cell expressing TRDV2, comprising contacting the T cell with a TRDV2 antibody provided herein. In certain embodiments, the contacting results in an increase in CD69, CD25, and/or Granzyme B expression, as compared to a control T cell expressing TRDV2.

In another aspect, provided herein is an antibody that binds TRGDC. In one embodiment, the TRGDC antibody comprises: In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4145; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4146. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4179; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4180. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4213; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4214. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4247; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4248. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4281; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4282. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4315; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4316. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4349; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4350. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4383; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4384. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4417; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4418. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4451; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4452. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4485; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4486. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4519; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4520. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4553; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4554. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4587; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4588. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4621; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4622. In one embodiment, the TRGDC antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4655; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4656.

In some embodiments, the VH CDR1, VH CDR2, VH CDR3, VL CDR1, VL CDR2, and VL CDR3 amino acid sequences of the TRGDC antibody are according to the Kabat numbering system. In some embodiments, the VH CDR1, VH CDR2, VH CDR3, VL CDR1, VL CDR2, and VL CDR3 amino acid sequences of the TRGDC antibody are according to the Chothia numbering system. In some embodiments, the VH CDR1, VH CDR2, VH CDR3, VL CDR1, VL CDR2, and VL CDR3 amino acid sequences of the TRGDC antibody are according to the AbM numbering system. In some embodiments, the VH CDR1, VH CDR2, VH CDR3, VL CDR1, VL CDR2, and VL CDR3 amino acid sequences of the TRGDC antibody are according to the Contact numbering system. In some embodiments, the VH CDR1, VH CDR2, VH CDR3, VL CDR1, VL CDR2, and VL CDR3 amino acid sequences of the TRGDC antibody are according to the IMGT numbering system.

In some embodiments, the antibody binds a TRGDC antigen. In some embodiments, antibody binds a TRGDC epitope. In some embodiments, the antibody specifically binds to TRGDC. In some embodiments, the VH CDR1, VH CDR2, VH CDR3, VL CDR1, VL CDR2 and VL CDR3 form a binding site for an antigen of the TRGDC. In some embodiments, the VH CDR1, VH CDR2, VH CDR3, VL CDR1, VL CDR2 and VL CDR3 form a binding site for an epitope of the TRGDC. In some embodiments, the TRGDC is present on the surface of a T cell.

In some embodiments, the TRGDC antibody is a humanized antibody. In some embodiments, the antibody is an IgG antibody. In some embodiments, the TRGDC antibody is an IgG1 antibody. In some embodiments, the TRGDC antibody is an IgG2 antibody. In some embodiments, the TRGDC antibody is an IgG3 antibody. In some embodiments, the TRGDC antibody is an IgG4 antibody. In some embodiments, the TRGDC antibody comprises a kappa light chain. In some embodiments, the TRGDC the antibody comprises a lambda light chain. In some embodiments, the TRGDC antibody is a monoclonal antibody. In some embodiments, the TRGDC antibody is multivalent. In some embodiments, the TRGDC antibody is capable of binding at least three antigens. In some embodiments, the TRGDC antibody is capable of binding at least five antigens. In some embodiments, the TRGDC antibody is a multispecific antibody. In some embodiments, the TRGDC antibody is a bispecific antibody.

In another aspect, provided is a nucleic acid sequence encoding a TRGDC antibody provided herein. Also provided is a vector comprising a nucleic acid sequence encoding a TRGDC antibody provided herein. Also provided is a host cell comprising a vector comprising a nucleic acid sequence encoding a TRGDC antibody provided herein. Also provided is a kit comprising vector comprising a nucleic acid sequence encoding a TRGDC antibody provided herein, and packaging for the same.

In another aspect, provided is a kit comprising a TRGDC antibody provided herein, and packaging for the same.

In another aspect, provided is a pharmaceutical composition comprising a TRGDC antibody provided herein, and a pharmaceutically acceptable carrier.

In another aspect, provided is a method of producing a pharmaceutical composition comprising a TRGDC antibody provided herein, comprising combining the TRGDC antibody with a pharmaceutically acceptable carrier to obtain the pharmaceutical composition.

In another aspect, provided is a method of activating a T cell expressing TRGDC, comprising contacting the T cell with a TRGDC antibody provided herein. In certain embodiments, the contacting results in an increase in CD69, CD25, and/or Granzyme B expression, as compared to a control T cell expressing TRGDC.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series.

Those skilled in the art will recognize or be able to ascertain using no more than routine experimentation, many equivalents to the specific embodiments described herein. Such equivalents are intended to been compassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc. In a specific embodiments, the subject is a human.

It should also be understood that the terms “about,” “approximately,” “generally,” “substantially,” and like terms, used herein when referring to a dimension or characteristic of a component of embodiments provided herein, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., TRGV9 antibody and polynucleotides that encode them, TRDV2 antibody and polynucleotides that encode them, or TRGDC antibody and polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule provided herein. The “host cell” can be any type of cell, e.g., a primary cell, a cell in culture, or a cell from a cell line. In one embodiment, a “host cell” is a cell transfected with a nucleic acid molecule provided herein. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

### Antibodies

Provided herein are TRGV9 antibodies or antigen-binding fragments thereof, nucleic acids and expression vectors encoding the antibodies, recombinant cells containing the vectors, and compositions comprising the antibodies. The antibodies disclosed herein possess one or more desirable functional properties, including but not limited to high-affinity binding to TRGV9 or high specificity to TRGV9. Also provided herein are TRDV2 antibodies or antigen-binding fragments thereof, nucleic acids and expression vectors encoding the antibodies, recombinant cells containing the vectors, and compositions comprising the antibodies. The antibodies disclosed herein possess one or more desirable functional properties, including but not limited to high-affinity binding to TRDV2 or high specificity to TRDV2. Also provided herein are antibodies or antigen-binding fragments thereof that bind TRGDC, nucleic acids and expression vectors encoding the antibodies, recombinant cells containing the vectors, and compositions comprising the antibodies. The antibodies disclosed herein possess one or more desirable functional properties, including but not limited to high-affinity binding to TRGDC or high specificity to TRGDC. Methods of making the antibodies, and methods of using the antibodies to treat diseases are also provided. In certain embodiments, the antibodies disclosed herein possess the ability to treat or prevent a disease or disorder when administered to a subject alone or in combination with other therapies.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies provided herein can be of any of the five major classes or corresponding sub-classes. In specific embodiments, the antibodies provided herein are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies provided herein can, in certain embodiments, contain a kappa light chain constant domain. The antibodies provided herein can, in certain embodiments, also contain a lambda light chain constant domain. According to particular embodiments, the antibodies provided herein include heavy and/or light chain constant regions from rat or human antibodies. In specific embodiments, the contant region is a human constant region.

In addition to the heavy and light constant domains, antibodies contain an antigen-binding region that is made up of a light chain variable region (VL) and a heavy chain variable region (VH), each of which contains three domains (i.e., complementarity determining regions 1 (CDR1), CDR2 and CDR3. A “CDR” refers to one of three hypervariable regions (HCDR1, HCDR2 or HCDR3) within the non-framework region of the immunoglobulin (Ig or antibody) VH β-sheet framework, or one of three hypervariable regions (LCDR1, LCDR2 or LCDR3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. CDR region sequences have also been defined by AbM, Contact and IMGT. Exemplary CDR region sequences are illustrated herein, for example, in the tables provided in the Examples below. The positions of CDRs within a canonical antibody variable region have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra (1997)). Such nomenclature is similarly well known to those skilled in the art.

The light chain variable region CDR1 domain is interchangeably referred to herein as LCDR1 or VL CDR1. The light chain variable region CDR2 domain is interchangeably referred to herein as LCDR2 or VL CDR2. The light chain variable region CDR3 domain is interchangeably referred to herein as LCDR3 or VL CDR3. The heavy chain variable region CDR1 domain is interchangeably referred to herein as HCDR1 or VH CDR1. The heavy chain variable region CDR2 domain is interchangeably referred to herein as HCDR2 or VH CDR2. The heavy chain variable region CDR1 domain is interchangeably referred to herein as HCDR3 or VH CDR3.

The term “hypervariable region”, such as a VH or VL, when used herein refers to the regions of an antibody variable region that are hypervariable in sequence and/or form structurally defined loops. Generally, antibodies comprise six hypervariable regions; three in the VH (HCDR1, HCDR2, HCDR3), and three in the VL (LCDR1, LCDR2, LCDR3). A number of hypervariable region delineations are in use and are encompassed herein. The “Kabat” CDRs are based on sequence variability and are the most commonly used (see, e.g., Kabat et al., Sequences of Proteins of Immunological Interest, 5th Ed. Public Health Service, National Institutes of Health, Bethesda, MD. (1991)). “Chothia” refers instead to the location of the structural loops (see, e.g., Chothia and Lesk, J. Mol. Biol. 196:901-917 (1987)). The end of the Chothia CDR-HCDR1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The “AbM” hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). “Contact” hypervariable regions are based on an analysis of the available complex crystal structures.

Recently, a universal numbering system has been developed and widely adopted, ImMunoGeneTics (IMGT) Information System® (Lafranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). An Exemplary system, shown herein, combines Kabat and Chothia.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (LCDR1), 46-56 or 50-56 (LCDR2) and 89-97 or 89-96 (LCDR3) in the VL and 26-35 or 26-35A (HCDR1), 50-65 or 49-65 (HCDR2) and 93-102, 94-102, or 95-102 (HCDR3) in the VH. CDR sequences, reflecting each of the above numbering schemes, are provided herein, including in Tables 2-7.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable region, which contains the antigen binding site. The constant region may contain the CH1, CH2 and CH3 regions of the heavy chain and the CL region of the light chain.

The term “framework” or “FR” residues are those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

As used herein, the term an “isolated antibody” refers to an antibody which is substantially free of other antibodies having different antigenic specificities (e.g., an isolated antibody that specifically binds to TRGV9 is substantially free of antibodies that do not bind to TRGV9, or an isolated antibody that specifically binds to TRDV2 is substantially free of antibodies that do not bind to TRDV2, or an isolated antibody that specifically binds to TRGDC is substantially free of antibodies that do not bind to TRGDC). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. Monoclonal antibodies provided herein can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope (e.g., an epitope on a TRGV9 antigen, an epitope on a TRDV2 antigen, or an epitope on TRGDC) and a second immunoglobulin variable domain sequence that has binding specificity for a second epitope. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. A “trispecific antibody” is a multispecific antibody that binds three distinct antigens, or three distinct epitopes within the same antigen. A “quadraspecific antibody” is a multispecific antibody that binds four distinct antigens, or four distinct epitopes within the same antigen.

As used herein, the term “TRGV9” refers to a polypeptide capable of forming a T cell receptor when expressed on the surface of γδ T cells. TRGV9-expressing γδ T cells are among the first T cells to develop in the human fetus and are the predominant γδ T cell subset in healthy adult peripheral blood cells. The terms “TRGV9,” “Vγ9,” and “VG9” are used interchangeably herein. The term “TRGV9” includes any TRGV9 variant, isoform, and species homolog, which is naturally expressed by cells (including T cells) or can be expressed on cells transfected with genes or cDNA encoding the polypeptide. In specific embodiments, the TRGV9 is a human TRGV9. An exemplary human TRGV9 amino acid sequence is provided by GenBank Accession Number NG_001336.2.

As used herein, an antibody that “specifically binds to TRGV9” refers to an antibody that binds to a TRGV9, preferably a human TRGV9, with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less.

As used herein, the term “TRDV2” refers to a polypeptide capable of forming a T cell receptor when expressed on the surface of γδ T cells. TRDV2-expressing γδ T cells are among the first T cells to develop in the human fetus and are the predominant γδ T cell subset in healthy adult peripheral blood cells. The terms “TRDV2,” “Vδ2,” and “VD2” are used interchangeably herein. The term “TRDV2” includes any TRDV2 variant, isoform, and species homolog, which is naturally expressed by cells (including T cells) or can be expressed on cells transfected with genes or cDNA encoding the polypeptide. In specific embodiments, the TRDV2 is a human TRDV2. An exemplary human TRDV2 amino acid sequence is provided by GenBank Accession Number NG_001332.3.

As used herein, an antibody that “specifically binds to TRDV2” refers to an antibody that binds to a TRDV2, such as a human TRDV2, with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹¹ M or less.

As used herein, the term the “TRGDC” refers to a polypeptide capable of forming a T cell receptor when expressed on the surface of γδ T cells. The terms “TRGDC” and “gamma delta constant” are used interchangeably herein. The term “TRGDC” includes any TRGDC variant, isoform, and species homolog, which is naturally expressed by cells (including T cells) or can be expressed on cells transfected with genes or cDNA encoding the polypeptide. In specific embodiments, the TRGDC is a human TRGDC. An exemplary human T cell receptor delta constant (TRDC) amino acid sequence is provided by GenBank Accession Number NG_001332.3, and an exemplary human T cell receptor gamma constant (TRGC) amino acid sequence is provided by GenBank Accession Number NC_000007.14 (TRGC1) or NC_000007.14 (TRGC2).

As used herein, an antibody that “specifically binds to TRGDC” refers to an antibody that binds to a TRGDC, such as a human TRGDC, with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less.

The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system. The smaller the value of the KD of an antibody, the higher affinity that the antibody binds to a target antigen.

In one aspect, provided herein is an antibody that binds to TRGV9. In some embodiments, provided herein is an antibody that binds to a TRGV9 antigen. In some embodiments, provided herein is an antibody that binds to a TRGV9 epitope.

In some embodiments, the antibody comprises a heavy chain variable region and a light chain variable region. In a some embodiments, the TRGV9 antibody is not a single domain antibody or nanobody. In some embodiments, the TRGV9 antibody is a humanized antibody. In some embodiments, the TRGV9 antibody is a fully human antibody.

In certain embodiments, provided herein is a TRGV9 antibody comprising a VH region, VL region, VH CDR1, VH CDR2, VH CDR3, VL CDR1, VL CDR2, and/or VL CDR3 of any one of the antibodies described herein. In some embodiments, provided herein is a TRGV9 antibody comprising a VH region of any one of the antibodies described herein. In some embodiments, provided herein is a TRGV9 antibody comprising a VL region of any one of the antibodies described herein. In some embodiments, provided herein is a TRGV9 antibody comprising a VH region of any one of the antibodies described herein, and a VL region of any one of the antibodies described herein. In some embodiments, provided herein is a TRGV9 antibody comprising a VH CDR1, VH CDR2, and VH CDR3 of any one of the antibodies described herein. In some embodiments, provided herein is a TRGV9 antibody comprising a VL CDR1, VL CDR2, and VL CDR3 of any one of the antibodies described herein. In some embodiments, provided herein is a TRGV9 antibody comprising a VH CDR1, VH CDR2, and VH CDR3 of any one of the antibodies described herein; and a VL CDR1, VL CDR2, and VL CDR3 of any one of the antibodies described herein. Representative VH and VL amino acid sequences, including VH CDR1, VH CDR2, VH CDR3, VL CDR1, VL CDR2 and VL CDR3 amino acid sequences, of TRGV9 antibodies provided herein are provided in the Tables 2-7.

In one aspect, provided herein is an antibody that binds to TRDV2. In some embodiments, provided herein is an antibody that binds to a TRDV2 antigen. In some embodiments, provided herein is an antibody that binds to a TRDV2 epitope.

In some embodiments, the antibody comprises a heavy chain variable region and a light chain variable region. In a some embodiments, the TRDV2 antibody is not a single domain antibody or nanobody. In some embodiments, the TRDV2 antibody is a humanized antibody. In some embodiments, the TRDV2 antibody is a fully human antibody.

In certain embodiments, provided herein is a TRDV2 antibody comprising a VH region, VL region, VH CDR1, VH CDR2, VH CDR3, VL CDR1, VL CDR2, and/or VL CDR3 of any one of the antibodies described herein. In some embodiments, provided herein is a TRDV2 antibody comprising a VH region of any one of the antibodies described herein. In some embodiments, provided herein is a TRDV2 antibody comprising a VL region of any one of the antibodies described herein. In some embodiments, provided herein is a TRDV2 antibody comprising a VH region of any one of the antibodies described herein, and a VL region of any one of the antibodies described herein. In some embodiments, provided herein is a TRDV2 antibody comprising a VH CDR1, VH CDR2, and VH CDR3 of any one of the antibodies described herein. In some embodiments, provided herein is a TRDV2 antibody comprising a VL CDR1, VL CDR2, and VL CDR3 of any one of the antibodies described herein. In some embodiments, provided herein is a TRDV2 antibody comprising a VH CDR1, VH CDR2, and VH CDR3 of any one of the antibodies described herein; and a VL CDR1, VL CDR2, and VL CDR3 of any one of the antibodies described herein. Representative VH and VL amino acid sequences, including VH CDR1, VH CDR2, VH CDR3, VL CDR1, VL CDR2 and VL CDR3 amino acid sequences, of TRDV2 antibodies provided herein are provided in Tables 2-7.

In one aspect, provided herein is an antibody that binds to TRGDC. In some embodiments, provided herein is an antibody that binds to a TRGDC antigen. In some embodiments, provided herein is an antibody that binds to a TRGDC epitope.

In some embodiments, the antibody comprises a heavy chain variable region and a light chain variable region. In a some embodiments, the TRGDC antibody is not a single domain antibody or nanobody. In some embodiments, the TRGDC antibody is a humanized antibody. In some embodiments, the TRGDC antibody is a fully human antibody.

In certain embodiments, provided herein is a TRGDC antibody comprising a VH region, VL region, VH CDR1, VH CDR2, VH CDR3, VL CDR1, VL CDR2, and/or VL CDR3 of any one of the antibodies described herein. In some embodiments, provided herein is a TRGDC antibody comprising a VH region of any one of the antibodies described herein. In some embodiments, provided herein is a TRGDC antibody comprising a VL region of any one of the antibodies described herein. In some embodiments, provided herein is a TRGDC antibody comprising a VH region of any one of the antibodies described herein, and a VL region of any one of the antibodies described herein. In some embodiments, provided herein is a TRGDC antibody comprising a VH CDR1, VH CDR2, and VH CDR3 of any one of the antibodies described herein. In some embodiments, provided herein is a TRGDC antibody comprising a VL CDR1, VL CDR2, and VL CDR3 of any one of the antibodies described herein. In some embodiments, provided herein is a TRGDC antibody comprising a VH CDR1, VH CDR2, and VH CDR3 of any one of the antibodies described herein; and a VL CDR1, VL CDR2, and VL CDR3 of any one of the antibodies described herein. Representative VH and VL amino acid sequences, including VH CDR1, VH CDR2, VH CDR3, VL CDR1, VL CDR2 and VL CDR3 amino acid sequences, of TRGDC antibodies provided herein are provided in Tables 2-7.

In some embodiments, the antibody specifically binds TRGV9. In other embodiments, the TRGV9 is present on the surface of a T cell. In some embodiments, the antibody specifically binds TRDV2. In other embodiments, the TRDV2 is present on the surface of a T cell. In some embodiments, the antibody specifically binds TRGDC. In other embodiments, the TRGDC is present on the surface of a T cell.

In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In some embodiments, the antibody is a trispecific antibody. In some embodiments, the antibody is a quadraspecific antibody. In certain embodiments, the antibody is multivalent. In other embodiments, the antibody is capable of binding at least three antigens. In some embodiments, the antibody is capable of binding at least four antigens. In some embodiments, the antibody is capable of binding at least five antigens.

In certain embodiments, provided is a TRGV9 antibody that is an intact antibody. In other embodiments, provided is a TRGV9 antibody is an antigen binding fragment of the TRGV9 antibody. In some embodiments, the antigen binding fragment of the TRGV9 antibody is a functional fragment. In certain embodiments, provided is a TRDV2 antibody that is an intact antibody. In other embodiments, provided is a TRDV2 antibody is an antigen binding fragment of the TRDV2 antibody. In some embodiments, the antigen binding fragment of the TRDV2 antibody is a functional fragment. In certain embodiments, provided is a TRGDC antibody that is an intact antibody. In other embodiments, provided is a TRGDC antibody is an antigen binding fragment of the TRGDC antibody. In some embodiments, the antigen binding fragment of the TRGDC antibody is a functional fragment.

In some embodiments, the antigen binding fragment is a diabody. In some embodiments, the antigen binding fragment is a Fab. In some embodiments, the antigen binding fragment is a Fab′. In some embodiments, the antigen binding fragment is a F(ab′)2. In some embodiments, the antigen binding fragment is a Fv fragment. In some embodiments, the antigen binding fragment is a disulfide stabilized Fv fragment (dsFv). In some embodiments, the antigen binding fragment is a (dsFv)₂. In some embodiments, the antigen binding fragment is a bispecific dsFv (dsFv-dsFv′). In some embodiments, the antigen binding fragment is a disulfide stabilized diabody (ds diabody). In some embodiments, the antigen binding fragment is a single-chain antibody molecule (scFv). In some embodiments, the antigen binding fragment is a single domain antibody (sdAb). In some embodiments, the antigen binding fragment is an scFv dimer (bivalent diabody). In some embodiments, the antigen binding fragment is a multispecific antibody formed from a portion of an antibody comprising one or more CDRs. In some embodiments, the antigen binding fragment is a camelized single domain antibody. In some embodiments, the antigen binding fragment is a nanobody. In some embodiments, the antigen binding fragment is a domain antibody. In some embodiments, the antigen binding fragment is a bivalent domain antibody. In some embodiments, the antigen binding fragment is an antibody fragment that binds to an antigen but does not comprise a complete antibody structure.

In specific embodiments, the TRGV9 antibody comprises a VH region and a VL region. In some embodiments, the TRGV9 antibody is a single chain antibody. In some embodiments, the TRGV9 antibody is a single domain antibody. In some embodiments, the TRGV9 antibody is a nanobody. In certain embodiments, the TRGV9 antibody is a VHH antibody. In certain embodiments, the TRGV9 antibody is a llama antibody. In some embodiments, the TRGV9 antibody is not a single chain antibody. In some embodiments, the TRGV9 antibody is not a single domain antibody. In some embodiments, the TRGV9 antibody is not a nanobody. In certain embodiments, the TRGV9 antibody is not a VHH antibody. In certain embodiments, the TRGV9 antibody is not a llama antibody. In some embodiments, the TRGV9 antibody is a multispecific antibody In other embodiments, the TRGV9 antibody is a bispecific antibody. In other embodiments, the TRGV9 antibody is a trispecific antibody. In other embodiments, the TRGV9 antibody is a quadraspecific antibody. In certain embodiments, the multispecific antibody comprises an antigen binding fragment of a TRGV9 antibody provided herein. In other embodiments, the bispecific antibody comprises an antigen binding fragment of a TRGV9 antibody provided herein. In other embodiments, the trispecific antibody comprises an antigen binding fragment of a TRGV9 antibody provided herein. In other embodiments, the quadraspecific antibody comprises an antigen binding fragment of a TRGV9 antibody provided herein. In In certain embodiments, the TRGV9 antibody activates T cells. In other embodiments, the TRGV9 antibody is an antagonistic antibody. In certain embodiments, the TRGV9 antibody inactivates T cells. In some embodiments, the TRGV9 antibody blocks activation of T cells. In some embodiments, the TRGV9 antibody modulates the activity of T cells. In some embodiments, the TRGV9 antibody neither activates or inactivates the activity of T cells. In specific embodiments, the T cells are human T cells.

In specific embodiments, the TRDV2 antibody comprises a VH region and a VL region. In some embodiments, the TRDV2 antibody is a single chain antibody. In some embodiments, the TRDV2 antibody is a single domain antibody. In some embodiments, the TRDV2 antibody is a nanobody. In certain embodiments, the TRDV2 antibody is a VHH antibody. In certain embodiments, the TRDV2 antibody is a llama antibody. In some embodiments, the TRDV2 antibody is not a single chain antibody. In some embodiments, the TRDV2 antibody is not a single domain antibody. In some embodiments, the TRDV2 antibody is not a nanobody. In certain embodiments, the TRDV2 antibody is not a VHH antibody. In certain embodiments, the TRDV2 antibody is not a llama antibody. In some embodiments, the TRDV2 antibody is a multispecific antibody In other embodiments, the TRDV2 antibody is a bispecific antibody. In other embodiments, the TRDV2 antibody is a trispecific antibody. In other embodiments, the TRDV2 antibody is a quadraspecific antibody. In certain embodiments, the multispecific antibody comprises an antigen binding fragment of a TRDV2 antibody provided herein. In other embodiments, the bispecific antibody comprises an antigen binding fragment of a TRDV2 antibody provided herein. In other embodiments, the trispecific antibody comprises an antigen binding fragment of a TRDV2 antibody provided herein. In other embodiments, the quadraspecific antibody comprises an antigen binding fragment of a TRDV2 antibody provided herein. In In certain embodiments, the TRDV2 antibody activates T cells. In other embodiments, the TRDV2 antibody is an antagonistic antibody. In certain embodiments, the TRDV2 antibody inactivates T cells. In some embodiments, the TRDV2 antibody blocks activation of T cells. In some embodiments, the TRDV2 antibody modulates the activity of T cells. In some embodiments, the TRDV2 antibody neither activates or inactivates the activity of T cells. In specific embodiments, the T cells are human T cells.

In specific embodiments, the TRGDC antibody comprises a VH region and a VL region. In some embodiments, the TRGDC antibody is a single chain antibody. In some embodiments, the TRGDC antibody is a single domain antibody. In some embodiments, the TRGDC antibody is a nanobody. In certain embodiments, the TRGDC antibody is a VHH antibody. In certain embodiments, the TRGDC antibody is a llama antibody. In some embodiments, the TRGDC antibody is not a single chain antibody. In some embodiments, the TRGDC antibody is not a single domain antibody. In some embodiments, the TRGDC antibody is not a nanobody. In certain embodiments, the TRGDC antibody is not a VHH antibody. In certain embodiments, the TRGDC antibody is not a llama antibody. In some embodiments, the TRGDC antibody is a multispecific antibody In other embodiments, the TRGDC antibody is a bispecific antibody. In other embodiments, the TRGDC antibody is a trispecific antibody. In other embodiments, the TRGDC antibody is a quadraspecific antibody. In certain embodiments, the multispecific antibody comprises an antigen binding fragment of a TRGDC antibody provided herein. In other embodiments, the bispecific antibody comprises an antigen binding fragment of a TRGDC antibody provided herein. In other embodiments, the trispecific antibody comprises an antigen binding fragment of a TRGDC antibody provided herein. In other embodiments, the quadraspecific antibody comprises an antigen binding fragment of a TRGDC antibody provided herein. In In certain embodiments, the TRGDC antibody activates T cells. In other embodiments, the TRGDC antibody is an antagonistic antibody. In certain embodiments, the TRGDC antibody inactivates T cells. In some embodiments, the TRGDC antibody blocks activation of T cells. In some embodiments, the TRGDC antibody modulates the activity of T cells. In some embodiments, the TRGDC antibody neither activates or inactivates the activity of T cells. In specific embodiments, the T cells are human T cells.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein. Other sets of CDRs are contemplated and within the scope of the antibody embodiments provided herein.

In one aspect, provided herein is an antibody that binds to TRGV9. In some embodiments, the antibody comprises a heavy chain variable region and a light chain variable region. In some embodiments, the TRGV9 antibody is a humanized antibody.

In one aspect, provided herein is an antibody that binds TRGV9, comprising: (i) a VH comprising a VH CDR1, a VH CDR2, and a VH CDR3 having an amino acid sequence of SEQ ID NOs:1, 2, and 3, respectively, and (ii) a VL comprising a VL CDR1, VL CDR2, and VL CDR3 having an amino acid sequence of SEQ ID NOs:4, 5, and 6, respectively. In one aspect, provided herein is an antibody that binds TRGV9, comprising: (i) a VH comprising a VH CDR1, a VH CDR2, and a VH CDR3 having an amino acid sequence of SEQ ID NOs:7, 8, and 9, respectively, and (ii) a VL comprising a VL CDR1, VL CDR2, and VL CDR3 having an amino acid sequence of SEQ ID NOs:10, 11, and 12, respectively. In one aspect, provided herein is an antibody that binds TRGV9, comprising: (i) a VH comprising a VH CDR1, a VH CDR2, and a VH CDR3 having an amino acid sequence of SEQ ID NOs:13, 14, and 15, respectively, and (ii) a VL comprising a VL CDR1, VL CDR2, and VL CDR3 having an amino acid sequence of SEQ ID NOs:16, 17, and 18, respectively. In one aspect, provided herein is an antibody that binds TRGV9, comprising: (i) a VH comprising a VH CDR1, a VH CDR2, and a VH CDR3 having an amino acid sequence of SEQ ID NOs:19, 20, and 21, respectively, and (ii) a VL comprising a VL CDR1, VL CDR2, and VL CDR3 having an amino acid sequence of SEQ ID NOs:22, 23, and 24, respectively. In one aspect, provided herein is an antibody that binds TRGV9, comprising: (i) a VH comprising a VH CDR1, a VH CDR2, and a VH CDR3 having an amino acid sequence of SEQ ID NOs:25, 26, and 27, respectively, and (ii) a VL comprising a VL CDR1, VL CDR2, and VL CDR3 having an amino acid sequence of SEQ ID NOs:28, 29, and 30, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of SEQ ID NO:32. In one aspect, provided herein is an antibody that binds TRGV9, comprising a VH having an amino acid sequence of SEQ ID NO:31. In one aspect, provided herein is an antibody that binds TRGV9, comprising a VL having an amino acid sequence of SEQ ID NO:32. In one aspect, provided herein is an antibody that binds TRGV9, comprising a VH having an amino acid sequence of SEQ ID NO:31, and a VL having an amino acid sequence of SEQ ID NO:32. In one aspect, provided herein is an antibody that binds TRGV9, comprising a heavy chain having an amino acid sequence of SEQ ID NO:33. In one aspect, provided herein is an antibody that binds TRGV9, comprising a light chain having an amino acid sequence of SEQ ID NO:34. In one aspect, provided herein is an antibody that binds TRGV9, comprising a heavy chain having an amino acid sequence of SEQ ID NO:33, and a light chain having an amino acid sequence of SEQ ID NO:34. In one aspect, provided herein is an antibody that binds TRGV9, comprising a VH having an amino acid sequence having at least 95% identity to an amino acid sequence of SEQ ID NO:31. In one aspect, provided herein is an antibody that binds TRGV9, comprising a VL having an amino acid sequence having at least 95% identity to an amino acid sequence of SEQ ID NO:32. In one aspect, provided herein is an antibody that binds TRGV9, comprising a VH having an amino acid sequence having at least 95% identity to an amino acid sequence of SEQ ID NO:31, and a VL having an amino acid sequence having at least 95% identity to an amino acid sequence of SEQ ID NO:32. In one aspect, provided herein is an antibody that binds TRGV9, comprising a heavy chain having an amino acid sequence having at least 95% identity to an amino acid sequence of SEQ ID NO:33. In one aspect, provided herein is an antibody that binds TRGV9, comprising a light chain having an amino acid sequence having at least 95% identity to an amino acid sequence of SEQ ID NO:34. In one aspect, provided herein is an antibody that binds TRGV9, comprising a heavy chain having an amino acid sequence having at least 95% identity to an amino acid sequence of SEQ ID NO:33, and a light chain having an amino acid sequence having at least 95% identity to an amino acid sequence of SEQ ID NO:34.

In one aspect, provided herein is an antibody that binds TRGV9, comprising: (i) a VH comprising a VH CDR1, a VH CDR2, and a VH CDR3 having an amino acid sequence of SEQ ID NOs:35, 36, and 37, respectively, and (ii) a VL comprising a VL CDR1, VL CDR2, and VL CDR3 having an amino acid sequence of SEQ ID NOs:38, 39, and 40, respectively. In one aspect, provided herein is an antibody that binds TRGV9, comprising: (i) a VH comprising a VH CDR1, a VH CDR2, and a VH CDR3 having an amino acid sequence of SEQ ID NOs:41, 42, and 43, respectively, and (ii) a VL comprising a VL CDR1, VL CDR2, and VL CDR3 having an amino acid sequence of SEQ ID NOs:44, 45, and 46, respectively. In one aspect, provided herein is an antibody that binds TRGV9, comprising: (i) a VH comprising a VH CDR1, a VH CDR2, and a VH CDR3 having an amino acid sequence of SEQ ID NOs:47, 48, and 49, respectively, and (ii) a VL comprising a VL CDR1, VL CDR2, and VL CDR3 having an amino acid sequence of SEQ ID NOs:50, 51, and 52, respectively. In one aspect, provided herein is an antibody that binds TRGV9, comprising: (i) a VH comprising a VH CDR1, a VH CDR2, and a VH CDR3 having an amino acid sequence of SEQ ID NOs:53, 54, and 55, respectively, and (ii) a VL comprising a VL CDR1, VL CDR2, and VL CDR3 having an amino acid sequence of SEQ ID NOs:56, 57, and 58, respectively. In one aspect, provided herein is an antibody that binds TRGV9, comprising: (i) a VH comprising a VH CDR1, a VH CDR2, and a VH CDR3 having an amino acid sequence of SEQ ID NOs:59, 60, and 61, respectively, and (ii) a VL comprising a VL CDR1, VL CDR2, and VL CDR3 having an amino acid sequence of SEQ ID NOs:62, 63, and 64, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of SEQ ID NO:66. In one aspect, provided herein is an antibody that binds TRGV9, comprising a VH having an amino acid sequence of SEQ ID NO:65. In one aspect, provided herein is an antibody that binds TRGV9, comprising a VL having an amino acid sequence of SEQ ID NO:66. In one aspect, provided herein is an antibody that binds TRGV9, comprising a VH having an amino acid sequence of SEQ ID NO:65, and a VL having an amino acid sequence of SEQ ID NO:66. In one aspect, provided herein is an antibody that binds TRGV9, comprising a heavy chain having an amino acid sequence of SEQ ID NO:67. In one aspect, provided herein is an antibody that binds TRGV9, comprising a light chain having an amino acid sequence of SEQ ID NO:68. In one aspect, provided herein is an antibody that binds TRGV9, comprising a heavy chain having an amino acid sequence of SEQ ID NO:67, and a light chain having an amino acid sequence of SEQ ID NO:68. In one aspect, provided herein is an antibody that binds TRGV9, comprising a VH having an amino acid sequence having at least 95% identity to an amino acid sequence of SEQ ID NO:65. In one aspect, provided herein is an antibody that binds TRGV9, comprising a VL having an amino acid sequence having at least 95% identity to an amino acid sequence of SEQ ID NO:66. In one aspect, provided herein is an antibody that binds TRGV9, comprising a VH having an amino acid sequence having at least 95% identity to an amino acid sequence of SEQ ID NO:65, and a VL having an amino acid sequence having at least 95% identity to an amino acid sequence of SEQ ID NO:66. In one aspect, provided herein is an antibody that binds TRGV9, comprising a heavy chain having an amino acid sequence having at least 95% identity to an amino acid sequence of SEQ ID NO:67. In one aspect, provided herein is an antibody that binds TRGV9, comprising a light chain having an amino acid sequence having at least 95% identity to an amino acid sequence of SEQ ID NO:68. In one aspect, provided herein is an antibody that binds TRGV9, comprising a heavy chain having an amino acid sequence having at least 95% identity to an amino acid sequence of SEQ ID NO:67, and a light chain having an amino acid sequence having at least 95% identity to an amino acid sequence of SEQ ID NO:68.

In one aspect, provided herein is an antibody that binds TRGV9, comprising: (i) a VH comprising a VH CDR1, a VH CDR2, and a VH CDR3 having an amino acid sequence of SEQ ID NOs:69, 70, and 71, respectively, and (ii) a VL comprising a VL CDR1, VL CDR2, and VL CDR3 having an amino acid sequence of SEQ ID NOs:72, 73, and 74, respectively. In one aspect, provided herein is an antibody that binds TRGV9, comprising: (i) a VH comprising a VH CDR1, a VH CDR2, and a VH CDR3 having an amino acid sequence of SEQ ID NOs:75, 76, and 77, respectively, and (ii) a VL comprising a VL CDR1, VL CDR2, and VL CDR3 having an amino acid sequence of SEQ ID NOs:78, 79, and 80, respectively. In one aspect, provided herein is an antibody that binds TRGV9, comprising: (i) a VH comprising a VH CDR1, a VH CDR2, and a VH CDR3 having an amino acid sequence of SEQ ID NOs:81, 82, and 83, respectively, and (ii) a VL comprising a VL CDR1, VL CDR2, and VL CDR3 having an amino acid sequence of SEQ ID NOs:84, 85, and 86, respectively. In one aspect, provided herein is an antibody that binds TRGV9, comprising: (i) a VH comprising a VH CDR1, a VH CDR2, and a VH CDR3 having an amino acid sequence of SEQ ID NOs:87, 88, and 89, respectively, and (ii) a VL comprising a VL CDR1, VL CDR2, and VL CDR3 having an amino acid sequence of SEQ ID NOs:90, 91, and 92, respectively. In one aspect, provided herein is an antibody that binds TRGV9, comprising: (i) a VH comprising a VH CDR1, a VH CDR2, and a VH CDR3 having an amino acid sequence of SEQ ID NOs:93, 94, and 95, respectively, and (ii) a VL comprising a VL CDR1, VL CDR2, and VL CDR3 having an amino acid sequence of SEQ ID NOs:96, 97, and 98, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of SEQ ID NO:100. In one aspect, provided herein is an antibody that binds TRGV9, comprising a VH having an amino acid sequence of SEQ ID NO:99. In one aspect, provided herein is an antibody that binds TRGV9, comprising a VL having an amino acid sequence of SEQ ID NO:100. In one aspect, provided herein is an antibody that binds TRGV9, comprising a VH having an amino acid sequence of SEQ ID NO:99, and a VL having an amino acid sequence of SEQ ID NO:100. In one aspect, provided herein is an antibody that binds TRGV9, comprising a heavy chain having an amino acid sequence of SEQ ID NO:101. In one aspect, provided herein is an antibody that binds TRGV9, comprising a light chain having an amino acid sequence of SEQ ID NO:102. In one aspect, provided herein is an antibody that binds TRGV9, comprising a heavy chain having an amino acid sequence of SEQ ID NO:101, and a light chain having an amino acid sequence of SEQ ID NO:102. In one aspect, provided herein is an antibody that binds TRGV9, comprising a VH having an amino acid sequence having at least 95% identity to an amino acid sequence of SEQ ID NO:99. In one aspect, provided herein is an antibody that binds TRGV9, comprising a VL having an amino acid sequence having at least 95% identity to an amino acid sequence of SEQ ID NO:100. In one aspect, provided herein is an antibody that binds TRGV9, comprising a VH having an amino acid sequence having at least 95% identity to an amino acid sequence of SEQ ID NO:99, and a VL having an amino acid sequence having at least 95% identity to an amino acid sequence of SEQ ID NO:100. In one aspect, provided herein is an antibody that binds TRGV9, comprising a heavy chain having an amino acid sequence having at least 95% identity to an amino acid sequence of SEQ ID NO:101. In one aspect, provided herein is an antibody that binds TRGV9, comprising a light chain having an amino acid sequence having at least 95% identity to an amino acid sequence of SEQ ID NO:102. In one aspect, provided herein is an antibody that binds TRGV9, comprising a heavy chain having an amino acid sequence having at least 95% identity to an amino acid sequence of SEQ ID NO:101, and a light chain having an amino acid sequence having at least 95% identity to an amino acid sequence of SEQ ID NO:102.

In one aspect, provided herein is an antibody that binds TRGV9, comprising: (i) a VH comprising a VH CDR1, a VH CDR2, and a VH CDR3 having an amino acid sequence of SEQ ID NOs:103, 104, and 105, respectively, and (ii) a VL comprising a VL CDR1, VL CDR2, and VL CDR3 having an amino acid sequence of SEQ ID NOs:106, 107, and 108, respectively. In one aspect, provided herein is an antibody that binds TRGV9, comprising: (i) a VH comprising a VH CDR1, a VH CDR2, and a VH CDR3 having an amino acid sequence of SEQ ID NOs:109, 110, and 111, respectively, and (ii) a VL comprising a VL CDR1, VL CDR2, and VL CDR3 having an amino acid sequence of SEQ ID NOs:112, 113, and 114, respectively. In one aspect, provided herein is an antibody that binds TRGV9, comprising: (i) a VH comprising a VH CDR1, a VH CDR2, and a VH CDR3 having an amino acid sequence of SEQ ID NOs:115, 116, and 117, respectively, and (ii) a VL comprising a VL CDR1, VL CDR2, and VL CDR3 having an amino acid sequence of SEQ ID NOs:118, 119, and 120, respectively. In one aspect, provided herein is an antibody that binds TRGV9, comprising: (i) a VH comprising a VH CDR1, a VH CDR2, and a VH CDR3 having an amino acid sequence of SEQ ID NOs:121, 122, and 123, respectively, and (ii) a VL comprising a VL CDR1, VL CDR2, and VL CDR3 having an amino acid sequence of SEQ ID NOs:124, 125, and 126, respectively. In one aspect, provided herein is an antibody that binds TRGV9, comprising: (i) a VH comprising a VH CDR1, a VH CDR2, and a VH CDR3 having an amino acid sequence of SEQ ID NOs:127, 128, and 129, respectively, and (ii) a VL comprising a VL CDR1, VL CDR2, and VL CDR3 having an amino acid sequence of SEQ ID NOs:130, 131, and 132, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133; and (ii) a VL comprising a VL CDR1, a VL CDR2, and a VL CDR3 having an amino acid sequence of a VL CDR1, a VL CDR2, and a VL CDR3, respectively, of SEQ ID NO:134. In one aspect, provided herein is an antibody that binds TRGV9, comprising a VH having an amino acid sequence of SEQ ID NO:133. In one aspect, provided herein is an antibody that binds TRGV9, comprising a VL having an amino acid sequence of SEQ ID NO:134. In one aspect, provided herein is an antibody that binds TRGV9, comprising a VH having an amino acid sequence of SEQ ID NO:133, and a VL having an amino acid sequence of SEQ ID NO:134. In one aspect, provided herein is an antibody that binds TRGV9, comprising a heavy chain having an amino acid sequence of SEQ ID NO:135. In one aspect, provided herein is an antibody that binds TRGV9, comprising a light chain having an amino acid sequence of SEQ ID NO:136. In one aspect, provided herein is an antibody that binds TRGV9, comprising a heavy chain having an amino acid sequence of SEQ ID NO:135, and a light chain having an amino acid sequence of SEQ ID NO:136. In one aspect, provided herein is an antibody that binds TRGV9, comprising a VH having an amino acid sequence having at least 95% identity to an amino acid sequence of SEQ ID NO:133. In one aspect, provided herein is an antibody that binds TRGV9, comprising a VL having an amino acid sequence having at least 95% identity to an amino acid sequence of SEQ ID NO:134. In one aspect, provided herein is an antibody that binds TRGV9, comprising a VH having an amino acid sequence having at least 95% identity to an amino acid sequence of SEQ ID NO:133, and a VL having an amino acid sequence having at least 95% identity to an amino acid sequence of SEQ ID NO:134. In one aspect, provided herein is an antibody that binds TRGV9, comprising a heavy chain having an amino acid sequence having at least 95% identity to an amino acid sequence of SEQ ID NO:135. In one aspect, provided herein is an antibody that binds TRGV9, comprising a light chain having an amino acid sequence having at least 95% identity to an amino acid sequence of SEQ ID NO:136. In one aspect, provided herein is an antibody that binds TRGV9, comprising a heavy chain having an amino acid sequence having at least 95% identity to an amino acid sequence of SEQ ID NO:135, and a light chain having an amino acid sequence having at least 95% identity to an amino acid sequence of SEQ ID NO:136.

In one aspect, provided herein is an antibody that binds TRGV9, comprising: (i) a VH comprising a VH CDR1, a VH CDR2, and a VH CDR3 having an amino acid sequence of SEQ ID NOs:137, 138, and 139, respectively, and (ii) a VL comprising a VL CDR1, VL CDR2, and VL CDR3 having an amino acid sequence of SEQ ID NOs:140, 141, and 142, respectively. In one aspect, provided herein is an antibody that binds TRGV9, comprising: (i) a VH comprising a VH CDR1, a VH CDR2, and a VH CDR3 having an amino acid sequence of SEQ ID NOs:143, 144, and 145, respectively, and (ii) a VL comprising a VL CDR1, VL CDR2, and VL CDR3 having an amino acid sequence of SEQ ID NOs:146, 147, and 148, respectively. In one aspect, provided herein is an antibody that binds TRGV9, comprising: (i) a VH comprising a VH CDR1, a VH CDR2, and a VH CDR3 having an amino acid sequence of SEQ ID NOs:149, 150, and 151, respectively, and (ii) a VL comprising a VL CDR1, VL CDR2, and VL CDR3 having an amino acid sequence of SEQ ID NOs:152, 153, and 154, respectively. In one aspect, provided herein is an antibody that binds TRGV9, comprising: (i) a VH comprising a VH CDR1, a VH CDR2, and a VH CDR3 having an amino acid sequence of SEQ ID NOs:155, 156, and 157, respectively, and (ii) a VL comprising a VL CDR1, VL CDR2, and VL CDR3 having an amino acid sequence of SEQ ID NOs:158, 159, and 160, respectively. In one aspect, provided herein is an antibody that binds TRGV9, comprising: (i) a VH comprising a VH CDR1, a VH CDR2, and a VH CDR3 having an amino acid sequence of SEQ ID NOs:161, 162, and 163, respectively, and (ii) a VL comprising a VL CDR1, VL CDR2, and VL CDR3 having an amino acid sequence of SEQ ID NOs:164, 165, and 166, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of SEQ ID NO:168. In one aspect, provided herein is an antibody that binds TRGV9, comprising a VH having an amino acid sequence of SEQ ID NO:167. In one aspect, provided herein is an antibody that binds TRGV9, comprising a VL having an amino acid sequence of SEQ ID NO:168. In one aspect, provided herein is an antibody that binds TRGV9, comprising a VH having an amino acid sequence of SEQ ID NO:167, and a VL having an amino acid sequence of SEQ ID NO:168. In one aspect, provided herein is an antibody that binds TRGV9, comprising a heavy chain having an amino acid sequence of SEQ ID NO:169. In one aspect, provided herein is an antibody that binds TRGV9, comprising a light chain having an amino acid sequence of SEQ ID NO:170. In one aspect, provided herein is an antibody that binds TRGV9, comprising a heavy chain having an amino acid sequence of SEQ ID NO:169, and a light chain having an amino acid sequence of SEQ ID NO:170. In one aspect, provided herein is an antibody that binds TRGV9, comprising a VH having an amino acid sequence having at least 95% identity to an amino acid sequence of SEQ ID NO:167. In one aspect, provided herein is an antibody that binds TRGV9, comprising a VL having an amino acid sequence having at least 95% identity to an amino acid sequence of SEQ ID NO:168. In one aspect, provided herein is an antibody that binds TRGV9, comprising a VH having an amino acid sequence having at least 95% identity to an amino acid sequence of SEQ ID NO:167, and a VL having an amino acid sequence having at least 95% identity to an amino acid sequence of SEQ ID NO:168. In one aspect, provided herein is an antibody that binds TRGV9, comprising a heavy chain having an amino acid sequence having at least 95% identity to an amino acid sequence of SEQ ID NO:169. In one aspect, provided herein is an antibody that binds TRGV9, comprising a light chain having an amino acid sequence having at least 95% identity to an amino acid sequence of SEQ ID NO:170. In one aspect, provided herein is an antibody that binds TRGV9, comprising a heavy chain having an amino acid sequence having at least 95% identity to an amino acid sequence of SEQ ID NO:169, and a light chain having an amino acid sequence having at least 95% identity to an amino acid sequence of SEQ ID NO:170.

In one aspect, provided herein is an antibody that binds TRGV9, comprising: (i) a VH comprising a VH CDR1, a VH CDR2, and a VH CDR3 having an amino acid sequence of SEQ ID NOs:171, 172, and 173, respectively, and (ii) a VL comprising a VL CDR1, VL CDR2, and VL CDR3 having an amino acid sequence of SEQ ID NOs:174, 175, and 176, respectively. In one aspect, provided herein is an antibody that binds TRGV9, comprising: (i) a VH comprising a VH CDR1, a VH CDR2, and a VH CDR3 having an amino acid sequence of SEQ ID NOs:177, 178, and 179, respectively, and (ii) a VL comprising a VL CDR1, VL CDR2, and VL CDR3 having an amino acid sequence of SEQ ID NOs:180, 181, and 182, respectively. In one aspect, provided herein is an antibody that binds TRGV9, comprising: (i) a VH comprising a VH CDR1, a VH CDR2, and a VH CDR3 having an amino acid sequence of SEQ ID NOs:183, 184, and 185, respectively, and (ii) a VL comprising a VL CDR1, VL CDR2, and VL CDR3 having an amino acid sequence of SEQ ID NOs:186, 187, and 188, respectively. In one aspect, provided herein is an antibody that binds TRGV9, comprising: (i) a VH comprising a VH CDR1, a VH CDR2, and a VH CDR3 having an amino acid sequence of SEQ ID NOs:189, 190, and 191, respectively, and (ii) a VL comprising a VL CDR1, VL CDR2, and VL CDR3 having an amino acid sequence of SEQ ID NOs:192, 193, and 194, respectively. In one aspect, provided herein is an antibody that binds TRGV9, comprising: (i) a VH comprising a VH CDR1, a VH CDR2, and a VH CDR3 having an amino acid sequence of SEQ ID NOs:195, 196, and 197, respectively, and (ii) a VL comprising a VL CDR1, VL CDR2, and VL CDR3 having an amino acid sequence of SEQ ID NOs:198, 199, and 200, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:201; and (ii) a VL comprising a VL CDR1, a VL CDR2, and a VL CDR3 having an amino acid sequence of a VL CDR1, a VL CDR2, and a VL CDR3, respectively, of SEQ ID NO:202. In one aspect, provided herein is an antibody that binds TRGV9, comprising a VH having an amino acid sequence of SEQ ID NO:201. In one aspect, provided herein is an antibody that binds TRGV9, comprising a VL having an amino acid sequence of SEQ ID NO:202. In one aspect, provided herein is an antibody that binds TRGV9, comprising a VH having an amino acid sequence of SEQ ID NO:201, and a VL having an amino acid sequence of SEQ ID NO:202. In one aspect, provided herein is an antibody that binds TRGV9, comprising a heavy chain having an amino acid sequence of SEQ ID NO:203. In one aspect, provided herein is an antibody that binds TRGV9, comprising a light chain having an amino acid sequence of SEQ ID NO:204. In one aspect, provided herein is an antibody that binds TRGV9, comprising a heavy chain having an amino acid sequence of SEQ ID NO:203, and a light chain having an amino acid sequence of SEQ ID NO:204. In one aspect, provided herein is an antibody that binds TRGV9, comprising a VH having an amino acid sequence having at least 95% identity to an amino acid sequence of SEQ ID NO:201. In one aspect, provided herein is an antibody that binds TRGV9, comprising a VL having an amino acid sequence having at least 95% identity to an amino acid sequence of SEQ ID NO:202. In one aspect, provided herein is an antibody that binds TRGV9, comprising a VH having an amino acid sequence having at least 95% identity to an amino acid sequence of SEQ ID NO:201, and a VL having an amino acid sequence having at least 95% identity to an amino acid sequence of SEQ ID NO:202. In one aspect, provided herein is an antibody that binds TRGV9, comprising a heavy chain having an amino acid sequence having at least 95% identity to an amino acid sequence of SEQ ID NO:203. In one aspect, provided herein is an antibody that binds TRGV9, comprising a light chain having an amino acid sequence having at least 95% identity to an amino acid sequence of SEQ ID NO:204. In one aspect, provided herein is an antibody that binds TRGV9, comprising a heavy chain having an amino acid sequence having at least 95% identity to an amino acid sequence of SEQ ID NO:203, and a light chain having an amino acid sequence having at least 95% identity to an amino acid sequence of SEQ ID NO:204.

In one aspect, provided herein is an antibody that binds TRGV9, comprising: (i) a VH comprising a VH CDR1, a VH CDR2, and a VH CDR3 having an amino acid sequence of SEQ ID NOs:205, 206, and 207, respectively, and (ii) a VL comprising a VL CDR1, VL CDR2, and VL CDR3 having an amino acid sequence of SEQ ID NOs:208, 209, and 210, respectively. In one aspect, provided herein is an antibody that binds TRGV9, comprising: (i) a VH comprising a VH CDR1, a VH CDR2, and a VH CDR3 having an amino acid sequence of SEQ ID NOs:211, 212, and 213, respectively, and (ii) a VL comprising a VL CDR1, VL CDR2, and VL CDR3 having an amino acid sequence of SEQ ID NOs:214, 215, and 216, respectively. In one aspect, provided herein is an antibody that binds TRGV9, comprising: (i) a VH comprising a VH CDR1, a VH CDR2, and a VH CDR3 having an amino acid sequence of SEQ ID NOs:217, 218, and 219, respectively, and (ii) a VL comprising a VL CDR1, VL CDR2, and VL CDR3 having an amino acid sequence of SEQ ID NOs:220, 221, and 222, respectively. In one aspect, provided herein is an antibody that binds TRGV9, comprising: (i) a VH comprising a VH CDR1, a VH CDR2, and a VH CDR3 having an amino acid sequence of SEQ ID NOs:223, 224, and 225, respectively, and (ii) a VL comprising a VL CDR1, VL CDR2, and VL CDR3 having an amino acid sequence of SEQ ID NOs:226, 227, and 228, respectively. In one aspect, provided herein is an antibody that binds TRGV9, comprising: (i) a VH comprising a VH CDR1, a VH CDR2, and a VH CDR3 having an amino acid sequence of SEQ ID NOs:229, 230, and 231, respectively, and (ii) a VL comprising a VL CDR1, VL CDR2, and VL CDR3 having an amino acid sequence of SEQ ID NOs:232, 233, and 234, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of SEQ ID NO:236. In one aspect, provided herein is an antibody that binds TRGV9, comprising a VH having an amino acid sequence of SEQ ID NO:235. In one aspect, provided herein is an antibody that binds TRGV9, comprising a VL having an amino acid sequence of SEQ ID NO:236. In one aspect, provided herein is an antibody that binds TRGV9, comprising a VH having an amino acid sequence of SEQ ID NO:235, and a VL having an amino acid sequence of SEQ ID NO:236. In one aspect, provided herein is an antibody that binds TRGV9, comprising a heavy chain having an amino acid sequence of SEQ ID NO:237. In one aspect, provided herein is an antibody that binds TRGV9, comprising a light chain having an amino acid sequence of SEQ ID NO:238. In one aspect, provided herein is an antibody that binds TRGV9, comprising a heavy chain having an amino acid sequence of SEQ ID NO:237, and a light chain having an amino acid sequence of SEQ ID NO:238. In one aspect, provided herein is an antibody that binds TRGV9, comprising a VH having an amino acid sequence having at least 95% identity to an amino acid sequence of SEQ ID NO:235. In one aspect, provided herein is an antibody that binds TRGV9, comprising a VL having an amino acid sequence having at least 95% identity to an amino acid sequence of SEQ ID NO:236. In one aspect, provided herein is an antibody that binds TRGV9, comprising a VH having an amino acid sequence having at least 95% identity to an amino acid sequence of SEQ ID NO:235, and a VL having an amino acid sequence having at least 95% identity to an amino acid sequence of SEQ ID NO:236. In one aspect, provided herein is an antibody that binds TRGV9, comprising a heavy chain having an amino acid sequence having at least 95% identity to an amino acid sequence of SEQ ID NO:237. In one aspect, provided herein is an antibody that binds TRGV9, comprising a light chain having an amino acid sequence having at least 95% identity to an amino acid sequence of SEQ ID NO:238. In one aspect, provided herein is an antibody that binds TRGV9, comprising a heavy chain having an amino acid sequence having at least 95% identity to an amino acid sequence of SEQ ID NO:237, and a light chain having an amino acid sequence having at least 95% identity to an amino acid sequence of SEQ ID NO:238.

In one aspect, provided herein is an antibody that binds TRGV9, comprising: (i) a VH comprising a VH CDR1, a VH CDR2, and a VH CDR3 having an amino acid sequence of SEQ ID NOs:239, 240, and 241, respectively, and (ii) a VL comprising a VL CDR1, VL CDR2, and VL CDR3 having an amino acid sequence of SEQ ID NOs:242, 243, and 244, respectively. In one aspect, provided herein is an antibody that binds TRGV9, comprising: (i) a VH comprising a VH CDR1, a VH CDR2, and a VH CDR3 having an amino acid sequence of SEQ ID NOs:245, 246, and 247, respectively, and (ii) a VL comprising a VL CDR1, VL CDR2, and VL CDR3 having an amino acid sequence of SEQ ID NOs:248, 249, and 250, respectively. In one aspect, provided herein is an antibody that binds TRGV9, comprising: (i) a VH comprising a VH CDR1, a VH CDR2, and a VH CDR3 having an amino acid sequence of SEQ ID NOs:251, 252, and 253, respectively, and (ii) a VL comprising a VL CDR1, VL CDR2, and VL CDR3 having an amino acid sequence of SEQ ID NOs:254, 255, and 256, respectively. In one aspect, provided herein is an antibody that binds TRGV9, comprising: (i) a VH comprising a VH CDR1, a VH CDR2, and a VH CDR3 having an amino acid sequence of SEQ ID NOs:257, 258, and 259, respectively, and (ii) a VL comprising a VL CDR1, VL CDR2, and VL CDR3 having an amino acid sequence of SEQ ID NOs:260, 261, and 262, respectively. In one aspect, provided herein is an antibody that binds TRGV9, comprising: (i) a VH comprising a VH CDR1, a VH CDR2, and a VH CDR3 having an amino acid sequence of SEQ ID NOs:263, 264, and 265, respectively, and (ii) a VL comprising a VL CDR1, VL CDR2, and VL CDR3 having an amino acid sequence of SEQ ID NOs:266, 267, and 268, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:269; and (ii) a VL comprising a VL CDR1, a VL CDR2, and a VL CDR3 having an amino acid sequence of a VL CDR1, a VL CDR2, and a VL CDR3, respectively, of SEQ ID NO:270. In one aspect, provided herein is an antibody that binds TRGV9, comprising a VH having an amino acid sequence of SEQ ID NO:269. In one aspect, provided herein is an antibody that binds TRGV9, comprising a VL having an amino acid sequence of SEQ ID NO:270. In one aspect, provided herein is an antibody that binds TRGV9, comprising a VH having an amino acid sequence of SEQ ID NO:269, and a VL having an amino acid sequence of SEQ ID NO:270. In one aspect, provided herein is an antibody that binds TRGV9, comprising a heavy chain having an amino acid sequence of SEQ ID NO:271. In one aspect, provided herein is an antibody that binds TRGV9, comprising a light chain having an amino acid sequence of SEQ ID NO:272. In one aspect, provided herein is an antibody that binds TRGV9, comprising a heavy chain having an amino acid sequence of SEQ ID NO:271, and a light chain having an amino acid sequence of SEQ ID NO:272. In one aspect, provided herein is an antibody that binds TRGV9, comprising a VH having an amino acid sequence having at least 95% identity to an amino acid sequence of SEQ ID NO:269. In one aspect, provided herein is an antibody that binds TRGV9, comprising a VL having an amino acid sequence having at least 95% identity to an amino acid sequence of SEQ ID NO:270. In one aspect, provided herein is an antibody that binds TRGV9, comprising a VH having an amino acid sequence having at least 95% identity to an amino acid sequence of SEQ ID NO:269, and a VL having an amino acid sequence having at least 95% identity to an amino acid sequence of SEQ ID NO:270. In one aspect, provided herein is an antibody that binds TRGV9, comprising a heavy chain having an amino acid sequence having at least 95% identity to an amino acid sequence of SEQ ID NO:271. In one aspect, provided herein is an antibody that binds TRGV9, comprising a light chain having an amino acid sequence having at least 95% identity to an amino acid sequence of SEQ ID NO:272. In one aspect, provided herein is an antibody that binds TRGV9, comprising a heavy chain having an amino acid sequence having at least 95% identity to an amino acid sequence of SEQ ID NO:271, and a light chain having an amino acid sequence having at least 95% identity to an amino acid sequence of SEQ ID NO:272.

In one aspect, provided herein is an antibody that binds TRGV9, comprising: (i) a VH comprising a VH CDR1, a VH CDR2, and a VH CDR3 having an amino acid sequence of SEQ ID NOs:273, 274, and 275, respectively, and (ii) a VL comprising a VL CDR1, VL CDR2, and VL CDR3 having an amino acid sequence of SEQ ID NOs:276, 277, and 278, respectively. In one aspect, provided herein is an antibody that binds TRGV9, comprising: (i) a VH comprising a VH CDR1, a VH CDR2, and a VH CDR3 having an amino acid sequence of SEQ ID NOs:279, 280, and 281, respectively, and (ii) a VL comprising a VL CDR1, VL CDR2, and VL CDR3 having an amino acid sequence of SEQ ID NOs:282, 283, and 284, respectively. In one aspect, provided herein is an antibody that binds TRGV9, comprising: (i) a VH comprising a VH CDR1, a VH CDR2, and a VH CDR3 having an amino acid sequence of SEQ ID NOs:285, 286, and 287, respectively, and (ii) a VL comprising a VL CDR1, VL CDR2, and VL CDR3 having an amino acid sequence of SEQ ID NOs:288, 289, and 290, respectively. In one aspect, provided herein is an antibody that binds TRGV9, comprising: (i) a VH comprising a VH CDR1, a VH CDR2, and a VH CDR3 having an amino acid sequence of SEQ ID NOs:291, 292, and 293, respectively, and (ii) a VL comprising a VL CDR1, VL CDR2, and VL CDR3 having an amino acid sequence of SEQ ID NOs:294, 295, and 296, respectively. In one aspect, provided herein is an antibody that binds TRGV9, comprising: (i) a VH comprising a VH CDR1, a VH CDR2, and a VH CDR3 having an amino acid sequence of SEQ ID NOs:297, 298, and 299, respectively, and (ii) a VL comprising a VL CDR1, VL CDR2, and VL CDR3 having an amino acid sequence of SEQ ID NOs:300, 301, and 302, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of SEQ ID NO:304. In one aspect, provided herein is an antibody that binds TRGV9, comprising a VH having an amino acid sequence of SEQ ID NO:303. In one aspect, provided herein is an antibody that binds TRGV9, comprising a VL having an amino acid sequence of SEQ ID NO:304. In one aspect, provided herein is an antibody that binds TRGV9, comprising a VH having an amino acid sequence of SEQ ID NO:303, and a VL having an amino acid sequence of SEQ ID NO:304. In one aspect, provided herein is an antibody that binds TRGV9, comprising a heavy chain having an amino acid sequence of SEQ ID NO:305. In one aspect, provided herein is an antibody that binds TRGV9, comprising a light chain having an amino acid sequence of SEQ ID NO:306. In one aspect, provided herein is an antibody that binds TRGV9, comprising a heavy chain having an amino acid sequence of SEQ ID NO:305, and a light chain having an amino acid sequence of SEQ ID NO:306. In one aspect, provided herein is an antibody that binds TRGV9, comprising a VH having an amino acid sequence having at least 95% identity to an amino acid sequence of SEQ ID NO:303. In one aspect, provided herein is an antibody that binds TRGV9, comprising a VL having an amino acid sequence having at least 95% identity to an amino acid sequence of SEQ ID NO:304. In one aspect, provided herein is an antibody that binds TRGV9, comprising a VH having an amino acid sequence having at least 95% identity to an amino acid sequence of SEQ ID NO:303, and a VL having an amino acid sequence having at least 95% identity to an amino acid sequence of SEQ ID NO:304. In one aspect, provided herein is an antibody that binds TRGV9, comprising a heavy chain having an amino acid sequence having at least 95% identity to an amino acid sequence of SEQ ID NO:305. In one aspect, provided herein is an antibody that binds TRGV9, comprising a light chain having an amino acid sequence having at least 95% identity to an amino acid sequence of SEQ ID NO:306. In one aspect, provided herein is an antibody that binds TRGV9, comprising a heavy chain having an amino acid sequence having at least 95% identity to an amino acid sequence of SEQ ID NO:305, and a light chain having an amino acid sequence having at least 95% identity to an amino acid sequence of SEQ ID NO:306.

In one aspect, provided herein is an antibody that binds TRGV9, comprising: (i) a VH comprising a VH CDR1, a VH CDR2, and a VH CDR3 having an amino acid sequence of SEQ ID NOs:307, 308, and 309, respectively, and (ii) a VL comprising a VL CDR1, VL CDR2, and VL CDR3 having an amino acid sequence of SEQ ID NOs:310, 311, and 312, respectively. In one aspect, provided herein is an antibody that binds TRGV9, comprising: (i) a VH comprising a VH CDR1, a VH CDR2, and a VH CDR3 having an amino acid sequence of SEQ ID NOs:313, 314, and 315, respectively, and (ii) a VL comprising a VL CDR1, VL CDR2, and VL CDR3 having an amino acid sequence of SEQ ID NOs:316, 317, and 318, respectively. In one aspect, provided herein is an antibody that binds TRGV9, comprising: (i) a VH comprising a VH CDR1, a VH CDR2, and a VH CDR3 having an amino acid sequence of SEQ ID NOs:319, 320, and 321, respectively, and (ii) a VL comprising a VL CDR1, VL CDR2, and VL CDR3 having an amino acid sequence of SEQ ID NOs:322, 323, and 324, respectively. In one aspect, provided herein is an antibody that binds TRGV9, comprising: (i) a VH comprising a VH CDR1, a VH CDR2, and a VH CDR3 having an amino acid sequence of SEQ ID NOs:325, 326, and 327, respectively, and (ii) a VL comprising a VL CDR1, VL CDR2, and VL CDR3 having an amino acid sequence of SEQ ID NOs:328, 329, and 330, respectively. In one aspect, provided herein is an antibody that binds TRGV9, comprising: (i) a VH comprising a VH CDR1, a VH CDR2, and a VH CDR3 having an amino acid sequence of SEQ ID NOs:331, 332, and 333, respectively, and (ii) a VL comprising a VL CDR1, VL CDR2, and VL CDR3 having an amino acid sequence of SEQ ID NOs:334, 335, and 336, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:337; and (ii) a VL comprising a VL CDR1, a VL CDR2, and a VL CDR3 having an amino acid sequence of a VL CDR1, a VL CDR2, and a VL CDR3, respectively, of SEQ ID NO:338. In one aspect, provided herein is an antibody that binds TRGV9, comprising a VH having an amino acid sequence of SEQ ID NO:337. In one aspect, provided herein is an antibody that binds TRGV9, comprising a VL having an amino acid sequence of SEQ ID NO:338. In one aspect, provided herein is an antibody that binds TRGV9, comprising a VH having an amino acid sequence of SEQ ID NO:337, and a VL having an amino acid sequence of SEQ ID NO:338. In one aspect, provided herein is an antibody that binds TRGV9, comprising a heavy chain having an amino acid sequence of SEQ ID NO:339. In one aspect, provided herein is an antibody that binds TRGV9, comprising a light chain having an amino acid sequence of SEQ ID NO:340. In one aspect, provided herein is an antibody that binds TRGV9, comprising a heavy chain having an amino acid sequence of SEQ ID NO:339, and a light chain having an amino acid sequence of SEQ ID NO:340. In one aspect, provided herein is an antibody that binds TRGV9, comprising a VH having an amino acid sequence having at least 95% identity to an amino acid sequence of SEQ ID NO:337. In one aspect, provided herein is an antibody that binds TRGV9, comprising a VL having an amino acid sequence having at least 95% identity to an amino acid sequence of SEQ ID NO:338. In one aspect, provided herein is an antibody that binds TRGV9, comprising a VH having an amino acid sequence having at least 95% identity to an amino acid sequence of SEQ ID NO:337, and a VL having an amino acid sequence having at least 95% identity to an amino acid sequence of SEQ ID NO:338. In one aspect, provided herein is an antibody that binds TRGV9, comprising a heavy chain having an amino acid sequence having at least 95% identity to an amino acid sequence of SEQ ID NO:339. In one aspect, provided herein is an antibody that binds TRGV9, comprising a light chain having an amino acid sequence having at least 95% identity to an amino acid sequence of SEQ ID NO:340. In one aspect, provided herein is an antibody that binds TRGV9, comprising a heavy chain having an amino acid sequence having at least 95% identity to an amino acid sequence of SEQ ID NO:339, and a light chain having an amino acid sequence having at least 95% identity to an amino acid sequence of SEQ ID NO:340.

In one aspect, provided herein is an antibody that binds TRGV9, comprising: (i) a VH comprising a VH CDR1, a VH CDR2, and a VH CDR3 having an amino acid sequence of SEQ ID NOs:341, 342, and 343, respectively, and (ii) a VL comprising a VL CDR1, VL CDR2, and VL CDR3 having an amino acid sequence of SEQ ID NOs:344, 345, and 346, respectively. In one aspect, provided herein is an antibody that binds TRGV9, comprising: (i) a VH comprising a VH CDR1, a VH CDR2, and a VH CDR3 having an amino acid sequence of SEQ ID NOs:347, 348, and 349, respectively, and (ii) a VL comprising a VL CDR1, VL CDR2, and VL CDR3 having an amino acid sequence of SEQ ID NOs:350, 351, and 352, respectively. In one aspect, provided herein is an antibody that binds TRGV9, comprising: (i) a VH comprising a VH CDR1, a VH CDR2, and a VH CDR3 having an amino acid sequence of SEQ ID NOs:353, 354, and 355, respectively, and (ii) a VL comprising a VL CDR1, VL CDR2, and VL CDR3 having an amino acid sequence of SEQ ID NOs:356, 357, and 358, respectively. In one aspect, provided herein is an antibody that binds TRGV9, comprising: (i) a VH comprising a VH CDR1, a VH CDR2, and a VH CDR3 having an amino acid sequence of SEQ ID NOs:359, 360, and 361, respectively, and (ii) a VL comprising a VL CDR1, VL CDR2, and VL CDR3 having an amino acid sequence of SEQ ID NOs:362, 363, and 364, respectively. In one aspect, provided herein is an antibody that binds TRGV9, comprising: (i) a VH comprising a VH CDR1, a VH CDR2, and a VH CDR3 having an amino acid sequence of SEQ ID NOs:365, 366, and 367, respectively, and (ii) a VL comprising a VL CDR1, VL CDR2, and VL CDR3 having an amino acid sequence of SEQ ID NOs:368, 369, and 370, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising a VL CDR1, a VL CDR2, and a VL CDR3 having an amino acid sequence of a VL CDR1, a VL CDR2, and a VL CDR3, respectively, of SEQ ID NO:372. In one aspect, provided herein is an antibody that binds TRGV9, comprising a VH having an amino acid sequence of SEQ ID NO:371. In one aspect, provided herein is an antibody that binds TRGV9, comprising a VL having an amino acid sequence of SEQ ID NO:372. In one aspect, provided herein is an antibody that binds TRGV9, comprising a VH having an amino acid sequence of SEQ ID NO:371, and a VL having an amino acid sequence of SEQ ID NO:372. In one aspect, provided herein is an antibody that binds TRGV9, comprising a heavy chain having an amino acid sequence of SEQ ID NO:373. In one aspect, provided herein is an antibody that binds TRGV9, comprising a light chain having an amino acid sequence of SEQ ID NO:374. In one aspect, provided herein is an antibody that binds TRGV9, comprising a heavy chain having an amino acid sequence of SEQ ID NO:373, and a light chain having an amino acid sequence of SEQ ID NO:374. In one aspect, provided herein is an antibody that binds TRGV9, comprising a VH having an amino acid sequence having at least 95% identity to an amino acid sequence of SEQ ID NO:371. In one aspect, provided herein is an antibody that binds TRGV9, comprising a VL having an amino acid sequence having at least 95% identity to an amino acid sequence of SEQ ID NO:372. In one aspect, provided herein is an antibody that binds TRGV9, comprising a VH having an amino acid sequence having at least 95% identity to an amino acid sequence of SEQ ID NO:371, and a VL having an amino acid sequence having at least 95% identity to an amino acid sequence of SEQ ID NO:372. In one aspect, provided herein is an antibody that binds TRGV9, comprising a heavy chain having an amino acid sequence having at least 95% identity to an amino acid sequence of SEQ ID NO:373. In one aspect, provided herein is an antibody that binds TRGV9, comprising a light chain having an amino acid sequence having at least 95% identity to an amino acid sequence of SEQ ID NO:374. In one aspect, provided herein is an antibody that binds TRGV9, comprising a heavy chain having an amino acid sequence having at least 95% identity to an amino acid sequence of SEQ ID NO:373, and a light chain having an amino acid sequence having at least 95% identity to an amino acid sequence of SEQ ID NO:374.

In one aspect, provided herein is an antibody that binds TRGV9, comprising: (i) a VH comprising a VH CDR1, a VH CDR2, and a VH CDR3 having an amino acid sequence of SEQ ID NOs:375, 376, and 377, respectively, and (ii) a VL comprising a VL CDR1, VL CDR2, and VL CDR3 having an amino acid sequence of SEQ ID NOs:378, 379, and 380, respectively. In one aspect, provided herein is an antibody that binds TRGV9, comprising: (i) a VH comprising a VH CDR1, a VH CDR2, and a VH CDR3 having an amino acid sequence of SEQ ID NOs:381, 382, and 383, respectively, and (ii) a VL comprising a VL CDR1, VL CDR2, and VL CDR3 having an amino acid sequence of SEQ ID NOs:384, 385, and 386, respectively. In one aspect, provided herein is an antibody that binds TRGV9, comprising: (i) a VH comprising a VH CDR1, a VH CDR2, and a VH CDR3 having an amino acid sequence of SEQ ID NOs:387, 388, and 389, respectively, and (ii) a VL comprising a VL CDR1, VL CDR2, and VL CDR3 having an amino acid sequence of SEQ ID NOs:390, 391, and 392, respectively. In one aspect, provided herein is an antibody that binds TRGV9, comprising: (i) a VH comprising a VH CDR1, a VH CDR2, and a VH CDR3 having an amino acid sequence of SEQ ID NOs:393, 394, and 395, respectively, and (ii) a VL comprising a VL CDR1, VL CDR2, and VL CDR3 having an amino acid sequence of SEQ ID NOs:396, 397, and 398, respectively. In one aspect, provided herein is an antibody that binds TRGV9, comprising: (i) a VH comprising a VH CDR1, a VH CDR2, and a VH CDR3 having an amino acid sequence of SEQ ID NOs:399, 400, and 401, respectively, and (ii) a VL comprising a VL CDR1, VL CDR2, and VL CDR3 having an amino acid sequence of SEQ ID NOs:402, 403, and 404, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of SEQ ID NO:406. In one aspect, provided herein is an antibody that binds TRGV9, comprising a VH having an amino acid sequence of SEQ ID NO:405. In one aspect, provided herein is an antibody that binds TRGV9, comprising a VL having an amino acid sequence of SEQ ID NO:406. In one aspect, provided herein is an antibody that binds TRGV9, comprising a VH having an amino acid sequence of SEQ ID NO:405, and a VL having an amino acid sequence of SEQ ID NO:406. In one aspect, provided herein is an antibody that binds TRGV9, comprising a heavy chain having an amino acid sequence of SEQ ID NO:407. In one aspect, provided herein is an antibody that binds TRGV9, comprising a light chain having an amino acid sequence of SEQ ID NO:408. In one aspect, provided herein is an antibody that binds TRGV9, comprising a heavy chain having an amino acid sequence of SEQ ID NO:407, and a light chain having an amino acid sequence of SEQ ID NO:408. In one aspect, provided herein is an antibody that binds TRGV9, comprising a VH having an amino acid sequence having at least 95% identity to an amino acid sequence of SEQ ID NO:405. In one aspect, provided herein is an antibody that binds TRGV9, comprising a VL having an amino acid sequence having at least 95% identity to an amino acid sequence of SEQ ID NO:406. In one aspect, provided herein is an antibody that binds TRGV9, comprising a VH having an amino acid sequence having at least 95% identity to an amino acid sequence of SEQ ID NO:405, and a VL having an amino acid sequence having at least 95% identity to an amino acid sequence of SEQ ID NO:406. In one aspect, provided herein is an antibody that binds TRGV9, comprising a heavy chain having an amino acid sequence having at least 95% identity to an amino acid sequence of SEQ ID NO:407. In one aspect, provided herein is an antibody that binds TRGV9, comprising a light chain having an amino acid sequence having at least 95% identity to an amino acid sequence of SEQ ID NO:408. In one aspect, provided herein is an antibody that binds TRGV9, comprising a heavy chain having an amino acid sequence having at least 95% identity to an amino acid sequence of SEQ ID NO:407, and a light chain having an amino acid sequence having at least 95% identity to an amino acid sequence of SEQ ID NO:408.

In one aspect, provided herein is an antibody that binds TRGV9, comprising: (i) a VH comprising a VH CDR1, a VH CDR2, and a VH CDR3 having an amino acid sequence of SEQ ID NOs:409, 410, and 411, respectively, and (ii) a VL comprising a VL CDR1, VL CDR2, and VL CDR3 having an amino acid sequence of SEQ ID NOs:412, 413, and 414, respectively. In one aspect, provided herein is an antibody that binds TRGV9, comprising: (i) a VH comprising a VH CDR1, a VH CDR2, and a VH CDR3 having an amino acid sequence of SEQ ID NOs:415, 416, and 417, respectively, and (ii) a VL comprising a VL CDR1, VL CDR2, and VL CDR3 having an amino acid sequence of SEQ ID NOs:418, 419, and 420, respectively. In one aspect, provided herein is an antibody that binds TRGV9, comprising: (i) a VH comprising a VH CDR1, a VH CDR2, and a VH CDR3 having an amino acid sequence of SEQ ID NOs:421, 422, and 423, respectively, and (ii) a VL comprising a VL CDR1, VL CDR2, and VL CDR3 having an amino acid sequence of SEQ ID NOs:424, 425, and 426, respectively. In one aspect, provided herein is an antibody that binds TRGV9, comprising: (i) a VH comprising a VH CDR1, a VH CDR2, and a VH CDR3 having an amino acid sequence of SEQ ID NOs:427, 428, and 429, respectively, and (ii) a VL comprising a VL CDR1, VL CDR2, and VL CDR3 having an amino acid sequence of SEQ ID NOs:430, 431, and 432, respectively. In one aspect, provided herein is an antibody that binds TRGV9, comprising: (i) a VH comprising a VH CDR1, a VH CDR2, and a VH CDR3 having an amino acid sequence of SEQ ID NOs:433, 434, and 435, respectively, and (ii) a VL comprising a VL CDR1, VL CDR2, and VL CDR3 having an amino acid sequence of SEQ ID NOs:436, 437, and 438, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of SEQ ID NO:440. In one aspect, provided herein is an antibody that binds TRGV9, comprising a VH having an amino acid sequence of SEQ ID NO:439. In one aspect, provided herein is an antibody that binds TRGV9, comprising a VL having an amino acid sequence of SEQ ID NO:440. In one aspect, provided herein is an antibody that binds TRGV9, comprising a VH having an amino acid sequence of SEQ ID NO:439, and a VL having an amino acid sequence of SEQ ID NO:440. In one aspect, provided herein is an antibody that binds TRGV9, comprising a heavy chain having an amino acid sequence of SEQ ID NO:441. In one aspect, provided herein is an antibody that binds TRGV9, comprising a light chain having an amino acid sequence of SEQ ID NO:442. In one aspect, provided herein is an antibody that binds TRGV9, comprising a heavy chain having an amino acid sequence of SEQ ID NO:441, and a light chain having an amino acid sequence of SEQ ID NO:442. In one aspect, provided herein is an antibody that binds TRGV9, comprising a VH having an amino acid sequence having at least 95% identity to an amino acid sequence of SEQ ID NO:439. In one aspect, provided herein is an antibody that binds TRGV9, comprising a VL having an amino acid sequence having at least 95% identity to an amino acid sequence of SEQ ID NO:440. In one aspect, provided herein is an antibody that binds TRGV9, comprising a VH having an amino acid sequence having at least 95% identity to an amino acid sequence of SEQ ID NO:439, and a VL having an amino acid sequence having at least 95% identity to an amino acid sequence of SEQ ID NO:440. In one aspect, provided herein is an antibody that binds TRGV9, comprising a heavy chain having an amino acid sequence having at least 95% identity to an amino acid sequence of SEQ ID NO:441. In one aspect, provided herein is an antibody that binds TRGV9, comprising a light chain having an amino acid sequence having at least 95% identity to an amino acid sequence of SEQ ID NO:442. In one aspect, provided herein is an antibody that binds TRGV9, comprising a heavy chain having an amino acid sequence having at least 95% identity to an amino acid sequence of SEQ ID NO:441, and a light chain having an amino acid sequence having at least 95% identity to an amino acid sequence of SEQ ID NO:442.

In one aspect, provided herein is an antibody that binds TRGV9, comprising: (i) a VH comprising a VH CDR1, a VH CDR2, and a VH CDR3 having an amino acid sequence of SEQ ID NOs:443, 444, and 445, respectively, and (ii) a VL comprising a VL CDR1, VL CDR2, and VL CDR3 having an amino acid sequence of SEQ ID NOs:446, 447, and 448, respectively. In one aspect, provided herein is an antibody that binds TRGV9, comprising: (i) a VH comprising a VH CDR1, a VH CDR2, and a VH CDR3 having an amino acid sequence of SEQ ID NOs:449, 450, and 451, respectively, and (ii) a VL comprising a VL CDR1, VL CDR2, and VL CDR3 having an amino acid sequence of SEQ ID NOs:452, 453, and 454, respectively. In one aspect, provided herein is an antibody that binds TRGV9, comprising: (i) a VH comprising a VH CDR1, a VH CDR2, and a VH CDR3 having an amino acid sequence of SEQ ID NOs:455, 456, and 457, respectively, and (ii) a VL comprising a VL CDR1, VL CDR2, and VL CDR3 having an amino acid sequence of SEQ ID NOs:458, 459, and 460, respectively. In one aspect, provided herein is an antibody that binds TRGV9, comprising: (i) a VH comprising a VH CDR1, a VH CDR2, and a VH CDR3 having an amino acid sequence of SEQ ID NOs:461, 462, and 463, respectively, and (ii) a VL comprising a VL CDR1, VL CDR2, and VL CDR3 having an amino acid sequence of SEQ ID NOs:464, 465, and 466, respectively. In one aspect, provided herein is an antibody that binds TRGV9, comprising: (i) a VH comprising a VH CDR1, a VH CDR2, and a VH CDR3 having an amino acid sequence of SEQ ID NOs:467, 468, and 469, respectively, and (ii) a VL comprising a VL CDR1, VL CDR2, and VL CDR3 having an amino acid sequence of SEQ ID NOs:470, 471, and 472, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising a VL CDR1, a VL CDR2, and a VL CDR3 having an amino acid sequence of a VL CDR1, a VL CDR2, and a VL CDR3, respectively, of SEQ ID NO:474. In one aspect, provided herein is an antibody that binds TRGV9, comprising a VH having an amino acid sequence of SEQ ID NO:473. In one aspect, provided herein is an antibody that binds TRGV9, comprising a VL having an amino acid sequence of SEQ ID NO:474. In one aspect, provided herein is an antibody that binds TRGV9, comprising a VH having an amino acid sequence of SEQ ID NO:473, and a VL having an amino acid sequence of SEQ ID NO:474. In one aspect, provided herein is an antibody that binds TRGV9, comprising a heavy chain having an amino acid sequence of SEQ ID NO:475. In one aspect, provided herein is an antibody that binds TRGV9, comprising a light chain having an amino acid sequence of SEQ ID NO:476. In one aspect, provided herein is an antibody that binds TRGV9, comprising a heavy chain having an amino acid sequence of SEQ ID NO:475, and a light chain having an amino acid sequence of SEQ ID NO:476. In one aspect, provided herein is an antibody that binds TRGV9, comprising a VH having an amino acid sequence having at least 95% identity to an amino acid sequence of SEQ ID NO:473. In one aspect, provided herein is an antibody that binds TRGV9, comprising a VL having an amino acid sequence having at least 95% identity to an amino acid sequence of SEQ ID NO:474. In one aspect, provided herein is an antibody that binds TRGV9, comprising a VH having an amino acid sequence having at least 95% identity to an amino acid sequence of SEQ ID NO:473, and a VL having an amino acid sequence having at least 95% identity to an amino acid sequence of SEQ ID NO:474. In one aspect, provided herein is an antibody that binds TRGV9, comprising a heavy chain having an amino acid sequence having at least 95% identity to an amino acid sequence of SEQ ID NO:475. In one aspect, provided herein is an antibody that binds TRGV9, comprising a light chain having an amino acid sequence having at least 95% identity to an amino acid sequence of SEQ ID NO:476. In one aspect, provided herein is an antibody that binds TRGV9, comprising a heavy chain having an amino acid sequence having at least 95% identity to an amino acid sequence of SEQ ID NO:475, and a light chain having an amino acid sequence having at least 95% identity to an amino acid sequence of SEQ ID NO:476.

In one aspect, provided herein is an antibody that binds TRGV9, comprising: (i) a VH comprising a VH CDR1, a VH CDR2, and a VH CDR3 having an amino acid sequence of SEQ ID NOs:477, 478, and 479, respectively, and (ii) a VL comprising a VL CDR1, VL CDR2, and VL CDR3 having an amino acid sequence of SEQ ID NOs:480, 481, and 482, respectively. In one aspect, provided herein is an antibody that binds TRGV9, comprising: (i) a VH comprising a VH CDR1, a VH CDR2, and a VH CDR3 having an amino acid sequence of SEQ ID NOs:483, 484, and 485, respectively, and (ii) a VL comprising a VL CDR1, VL CDR2, and VL CDR3 having an amino acid sequence of SEQ ID NOs:486, 487, and 488, respectively. In one aspect, provided herein is an antibody that binds TRGV9, comprising: (i) a VH comprising a VH CDR1, a VH CDR2, and a VH CDR3 having an amino acid sequence of SEQ ID NOs:489, 490, and 491, respectively, and (ii) a VL comprising a VL CDR1, VL CDR2, and VL CDR3 having an amino acid sequence of SEQ ID NOs:492, 493, and 494, respectively. In one aspect, provided herein is an antibody that binds TRGV9, comprising: (i) a VH comprising a VH CDR1, a VH CDR2, and a VH CDR3 having an amino acid sequence of SEQ ID NOs:495, 496, and 497, respectively, and (ii) a VL comprising a VL CDR1, VL CDR2, and VL CDR3 having an amino acid sequence of SEQ ID NOs:498, 499, and 500, respectively. In one aspect, provided herein is an antibody that binds TRGV9, comprising: (i) a VH comprising a VH CDR1, a VH CDR2, and a VH CDR3 having an amino acid sequence of SEQ ID NOs:501, 502, and 503, respectively, and (ii) a VL comprising a VL CDR1, VL CDR2, and VL CDR3 having an amino acid sequence of SEQ ID NOs:504, 505, and 506, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising a VL CDR1, a VL CDR2, and a VL CDR3 having an amino acid sequence of a VL CDR1, a VL CDR2, and a VL CDR3, respectively, of SEQ ID NO:508. In one aspect, provided herein is an antibody that binds TRGV9, comprising a VH having an amino acid sequence of SEQ ID NO:507. In one aspect, provided herein is an antibody that binds TRGV9, comprising a VL having an amino acid sequence of SEQ ID NO:508. In one aspect, provided herein is an antibody that binds TRGV9, comprising a VH having an amino acid sequence of SEQ ID NO:507, and a VL having an amino acid sequence of SEQ ID NO:508. In one aspect, provided herein is an antibody that binds TRGV9, comprising a heavy chain having an amino acid sequence of SEQ ID NO:509. In one aspect, provided herein is an antibody that binds TRGV9, comprising a light chain having an amino acid sequence of SEQ ID NO:510. In one aspect, provided herein is an antibody that binds TRGV9, comprising a heavy chain having an amino acid sequence of SEQ ID NO:509, and a light chain having an amino acid sequence of SEQ ID NO:510. In one aspect, provided herein is an antibody that binds TRGV9, comprising a VH having an amino acid sequence having at least 95% identity to an amino acid sequence of SEQ ID NO:507. In one aspect, provided herein is an antibody that binds TRGV9, comprising a VL having an amino acid sequence having at least 95% identity to an amino acid sequence of SEQ ID NO:508. In one aspect, provided herein is an antibody that binds TRGV9, comprising a VH having an amino acid sequence having at least 95% identity to an amino acid sequence of SEQ ID NO:507, and a VL having an amino acid sequence having at least 95% identity to an amino acid sequence of SEQ ID NO:508. In one aspect, provided herein is an antibody that binds TRGV9, comprising a heavy chain having an amino acid sequence having at least 95% identity to an amino acid sequence of SEQ ID NO:509. In one aspect, provided herein is an antibody that binds TRGV9, comprising a light chain having an amino acid sequence having at least 95% identity to an amino acid sequence of SEQ ID NO:510. In one aspect, provided herein is an antibody that binds TRGV9, comprising a heavy chain having an amino acid sequence having at least 95% identity to an amino acid sequence of SEQ ID NO:509, and a light chain having an amino acid sequence having at least 95% identity to an amino acid sequence of SEQ ID NO:510.

In one aspect, provided herein is an antibody that binds TRGV9, comprising: (i) a VH comprising a VH CDR1, a VH CDR2, and a VH CDR3 having an amino acid sequence of SEQ ID NOs:511, 512, and 513, respectively, and (ii) a VL comprising a VL CDR1, VL CDR2, and VL CDR3 having an amino acid sequence of SEQ ID NOs:514, 515, and 516, respectively. In one aspect, provided herein is an antibody that binds TRGV9, comprising: (i) a VH comprising a VH CDR1, a VH CDR2, and a VH CDR3 having an amino acid sequence of SEQ ID NOs:517, 518, and 519, respectively, and (ii) a VL comprising a VL CDR1, VL CDR2, and VL CDR3 having an amino acid sequence of SEQ ID NOs:520, 521, and 522, respectively. In one aspect, provided herein is an antibody that binds TRGV9, comprising: (i) a VH comprising a VH CDR1, a VH CDR2, and a VH CDR3 having an amino acid sequence of SEQ ID NOs:523, 524, and 525, respectively, and (ii) a VL comprising a VL CDR1, VL CDR2, and VL CDR3 having an amino acid sequence of SEQ ID NOs:526, 527, and 528, respectively. In one aspect, provided herein is an antibody that binds TRGV9, comprising: (i) a VH comprising a VH CDR1, a VH CDR2, and a VH CDR3 having an amino acid sequence of SEQ ID NOs:529, 530, and 531, respectively, and (ii) a VL comprising a VL CDR1, VL CDR2, and VL CDR3 having an amino acid sequence of SEQ ID NOs:532, 533, and 534, respectively. In one aspect, provided herein is an antibody that binds TRGV9, comprising: (i) a VH comprising a VH CDR1, a VH CDR2, and a VH CDR3 having an amino acid sequence of SEQ ID NOs:535, 536, and 537, respectively, and (ii) a VL comprising a VL CDR1, VL CDR2, and VL CDR3 having an amino acid sequence of SEQ ID NOs:538, 539, and 540, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising a VL CDR1, a VL CDR2, and a VL CDR3 having an amino acid sequence of a VL CDR1, a VL CDR2, and a VL CDR3, respectively, of SEQ ID NO:542. In one aspect, provided herein is an antibody that binds TRGV9, comprising a VH having an amino acid sequence of SEQ ID NO:541. In one aspect, provided herein is an antibody that binds TRGV9, comprising a VL having an amino acid sequence of SEQ ID NO:542. In one aspect, provided herein is an antibody that binds TRGV9, comprising a VH having an amino acid sequence of SEQ ID NO:541, and a VL having an amino acid sequence of SEQ ID NO:542. In one aspect, provided herein is an antibody that binds TRGV9, comprising a heavy chain having an amino acid sequence of SEQ ID NO:543. In one aspect, provided herein is an antibody that binds TRGV9, comprising a light chain having an amino acid sequence of SEQ ID NO:544. In one aspect, provided herein is an antibody that binds TRGV9, comprising a heavy chain having an amino acid sequence of SEQ ID NO:543, and a light chain having an amino acid sequence of SEQ ID NO:544. In one aspect, provided herein is an antibody that binds TRGV9, comprising a VH having an amino acid sequence having at least 95% identity to an amino acid sequence of SEQ ID NO:541. In one aspect, provided herein is an antibody that binds TRGV9, comprising a VL having an amino acid sequence having at least 95% identity to an amino acid sequence of SEQ ID NO:542. In one aspect, provided herein is an antibody that binds TRGV9, comprising a VH having an amino acid sequence having at least 95% identity to an amino acid sequence of SEQ ID NO:541, and a VL having an amino acid sequence having at least 95% identity to an amino acid sequence of SEQ ID NO:542. In one aspect, provided herein is an antibody that binds TRGV9, comprising a heavy chain having an amino acid sequence having at least 95% identity to an amino acid sequence of SEQ ID NO:543. In one aspect, provided herein is an antibody that binds TRGV9, comprising a light chain having an amino acid sequence having at least 95% identity to an amino acid sequence of SEQ ID NO:544. In one aspect, provided herein is an antibody that binds TRGV9, comprising a heavy chain having an amino acid sequence having at least 95% identity to an amino acid sequence of SEQ ID NO:543, and a light chain having an amino acid sequence having at least 95% identity to an amino acid sequence of SEQ ID NO:544.

In one aspect, provided herein is an antibody that binds TRGV9, comprising: (i) a VH comprising a VH CDR1, a VH CDR2, and a VH CDR3 having an amino acid sequence of SEQ ID NOs:545, 546, and 547, respectively, and (ii) a VL comprising a VL CDR1, VL CDR2, and VL CDR3 having an amino acid sequence of SEQ ID NOs:548,549, and 550, respectively. In one aspect, provided herein is an antibody that binds TRGV9, comprising: (i) a VH comprising a VH CDR1, a VH CDR2, and a VH CDR3 having an amino acid sequence of SEQ ID NOs:551, 552, and 553, respectively, and (ii) a VL comprising a VL CDR1, VL CDR2, and VL CDR3 having an amino acid sequence of SEQ ID NOs:554, 555, and 556, respectively. In one aspect, provided herein is an antibody that binds TRGV9, comprising: (i) a VH comprising a VH CDR1, a VH CDR2, and a VH CDR3 having an amino acid sequence of SEQ ID NOs:557, 558, and 559, respectively, and (ii) a VL comprising a VL CDR1, VL CDR2, and VL CDR3 having an amino acid sequence of SEQ ID NOs:560, 561, and 562, respectively. In one aspect, provided herein is an antibody that binds TRGV9, comprising: (i) a VH comprising a VH CDR1, a VH CDR2, and a VH CDR3 having an amino acid sequence of SEQ ID NOs:563, 564, and 565, respectively, and (ii) a VL comprising a VL CDR1, VL CDR2, and VL CDR3 having an amino acid sequence of SEQ ID NOs:566, 567, and 568, respectively. In one aspect, provided herein is an antibody that binds TRGV9, comprising: (i) a VH comprising a VH CDR1, a VH CDR2, and a VH CDR3 having an amino acid sequence of SEQ ID NOs:569, 570, and 571, respectively, and (ii) a VL comprising a VL CDR1, VL CDR2, and VL CDR3 having an amino acid sequence of SEQ ID NOs:572, 573, and 574, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising a VL CDR1, a VL CDR2, and a VL CDR3 having an amino acid sequence of a VL CDR1, a VL CDR2, and a VL CDR3, respectively, of SEQ ID NO:576. In one aspect, provided herein is an antibody that binds TRGV9, comprising a VH having an amino acid sequence of SEQ ID NO:575. In one aspect, provided herein is an antibody that binds TRGV9, comprising a VL having an amino acid sequence of SEQ ID NO:576. In one aspect, provided herein is an antibody that binds TRGV9, comprising a VH having an amino acid sequence of SEQ ID NO:575, and a VL having an amino acid sequence of SEQ ID NO:576. In one aspect, provided herein is an antibody that binds TRGV9, comprising a heavy chain having an amino acid sequence of SEQ ID NO:577. In one aspect, provided herein is an antibody that binds TRGV9, comprising a light chain having an amino acid sequence of SEQ ID NO:578. In one aspect, provided herein is an antibody that binds TRGV9, comprising a heavy chain having an amino acid sequence of SEQ ID NO:577, and a light chain having an amino acid sequence of SEQ ID NO:578. In one aspect, provided herein is an antibody that binds TRGV9, comprising a VH having an amino acid sequence having at least 95% identity to an amino acid sequence of SEQ ID NO:575. In one aspect, provided herein is an antibody that binds TRGV9, comprising a VL having an amino acid sequence having at least 95% identity to an amino acid sequence of SEQ ID NO:576. In one aspect, provided herein is an antibody that binds TRGV9, comprising a VH having an amino acid sequence having at least 95% identity to an amino acid sequence of SEQ ID NO:575, and a VL having an amino acid sequence having at least 95% identity to an amino acid sequence of SEQ ID NO:576. In one aspect, provided herein is an antibody that binds TRGV9, comprising a heavy chain having an amino acid sequence having at least 95% identity to an amino acid sequence of SEQ ID NO:577. In one aspect, provided herein is an antibody that binds TRGV9, comprising a light chain having an amino acid sequence having at least 95% identity to an amino acid sequence of SEQ ID NO:578. In one aspect, provided herein is an antibody that binds TRGV9, comprising a heavy chain having an amino acid sequence having at least 95% identity to an amino acid sequence of SEQ ID NO:577, and a light chain having an amino acid sequence having at least 95% identity to an amino acid sequence of SEQ ID NO:578.

In one aspect, provided herein is an antibody that binds TRGV9, comprising: (i) a VH comprising a VH CDR1, a VH CDR2, and a VH CDR3 having an amino acid sequence of SEQ ID NOs:579, 580, and 581, respectively, and (ii) a VL comprising a VL CDR1, VL CDR2, and VL CDR3 having an amino acid sequence of SEQ ID NOs:582, 583, and 584, respectively. In one aspect, provided herein is an antibody that binds TRGV9, comprising: (i) a VH comprising a VH CDR1, a VH CDR2, and a VH CDR3 having an amino acid sequence of SEQ ID NOs:585, 586, and 587, respectively, and (ii) a VL comprising a VL CDR1, VL CDR2, and VL CDR3 having an amino acid sequence of SEQ ID NOs:588, 589, and 590, respectively. In one aspect, provided herein is an antibody that binds TRGV9, comprising: (i) a VH comprising a VH CDR1, a VH CDR2, and a VH CDR3 having an amino acid sequence of SEQ ID NOs:591, 592, and 593, respectively, and (ii) a VL comprising a VL CDR1, VL CDR2, and VL CDR3 having an amino acid sequence of SEQ ID NOs:594, 595, and 596, respectively. In one aspect, provided herein is an antibody that binds TRGV9, comprising: (i) a VH comprising a VH CDR1, a VH CDR2, and a VH CDR3 having an amino acid sequence of SEQ ID NOs:597, 598, and 599, respectively, and (ii) a VL comprising a VL CDR1, VL CDR2, and VL CDR3 having an amino acid sequence of SEQ ID NOs:600, 601, and 602, respectively. In one aspect, provided herein is an antibody that binds TRGV9, comprising: (i) a VH comprising a VH CDR1, a VH CDR2, and a VH CDR3 having an amino acid sequence of SEQ ID NOs:603, 604, and 605, respectively, and (ii) a VL comprising a VL CDR1, VL CDR2, and VL CDR3 having an amino acid sequence of SEQ ID NOs:606, 607, and 608, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:609; and (ii) a VL comprising a VL CDR1, a VL CDR2, and a VL CDR3 having an amino acid sequence of a VL CDR1, a VL CDR2, and a VL CDR3, respectively, of SEQ ID NO:610. In one aspect, provided herein is an antibody that binds TRGV9, comprising a VH having an amino acid sequence of SEQ ID NO:609. In one aspect, provided herein is an antibody that binds TRGV9, comprising a VL having an amino acid sequence of SEQ ID NO:610. In one aspect, provided herein is an antibody that binds TRGV9, comprising a VH having an amino acid sequence of SEQ ID NO:609, and a VL having an amino acid sequence of SEQ ID NO:610. In one aspect, provided herein is an antibody that binds TRGV9, comprising a heavy chain having an amino acid sequence of SEQ ID NO:611. In one aspect, provided herein is an antibody that binds TRGV9, comprising a light chain having an amino acid sequence of SEQ ID NO:612. In one aspect, provided herein is an antibody that binds TRGV9, comprising a heavy chain having an amino acid sequence of SEQ ID NO:611, and a light chain having an amino acid sequence of SEQ ID NO:612. In one aspect, provided herein is an antibody that binds TRGV9, comprising a VH having an amino acid sequence having at least 95% identity to an amino acid sequence of SEQ ID NO:609. In one aspect, provided herein is an antibody that binds TRGV9, comprising a VL having an amino acid sequence having at least 95% identity to an amino acid sequence of SEQ ID NO:610. In one aspect, provided herein is an antibody that binds TRGV9, comprising a VH having an amino acid sequence having at least 95% identity to an amino acid sequence of SEQ ID NO:609, and a VL having an amino acid sequence having at least 95% identity to an amino acid sequence of SEQ ID NO:610. In one aspect, provided herein is an antibody that binds TRGV9, comprising a heavy chain having an amino acid sequence having at least 95% identity to an amino acid sequence of SEQ ID NO:611. In one aspect, provided herein is an antibody that binds TRGV9, comprising a light chain having an amino acid sequence having at least 95% identity to an amino acid sequence of SEQ ID NO:612. In one aspect, provided herein is an antibody that binds TRGV9, comprising a heavy chain having an amino acid sequence having at least 95% identity to an amino acid sequence of SEQ ID NO:611, and a light chain having an amino acid sequence having at least 95% identity to an amino acid sequence of SEQ ID NO:612.

In one aspect, provided herein is an antibody that binds TRGV9, comprising: (i) a VH comprising a VH CDR1, a VH CDR2, and a VH CDR3 having an amino acid sequence of SEQ ID NOs:613, 614, and 615, respectively, and (ii) a VL comprising a VL CDR1, VL CDR2, and VL CDR3 having an amino acid sequence of SEQ ID NOs:616, 617, and 618, respectively. In one aspect, provided herein is an antibody that binds TRGV9, comprising: (i) a VH comprising a VH CDR1, a VH CDR2, and a VH CDR3 having an amino acid sequence of SEQ ID NOs:619, 620, and 621, respectively, and (ii) a VL comprising a VL CDR1, VL CDR2, and VL CDR3 having an amino acid sequence of SEQ ID NOs:622, 523, and 624, respectively. In one aspect, provided herein is an antibody that binds TRGV9, comprising: (i) a VH comprising a VH CDR1, a VH CDR2, and a VH CDR3 having an amino acid sequence of SEQ ID NOs:625, 626, and 627, respectively, and (ii) a VL comprising a VL CDR1, VL CDR2, and VL CDR3 having an amino acid sequence of SEQ ID NOs:628, 629, and 630, respectively. In one aspect, provided herein is an antibody that binds TRGV9, comprising: (i) a VH comprising a VH CDR1, a VH CDR2, and a VH CDR3 having an amino acid sequence of SEQ ID NOs:631, 632, and 633, respectively, and (ii) a VL comprising a VL CDR1, VL CDR2, and VL CDR3 having an amino acid sequence of SEQ ID NOs:634, 635, and 636, respectively. In one aspect, provided herein is an antibody that binds TRGV9, comprising: (i) a VH comprising a VH CDR1, a VH CDR2, and a VH CDR3 having an amino acid sequence of SEQ ID NOs:637, 638, and 639, respectively, and (ii) a VL comprising a VL CDR1, VL CDR2, and VL CDR3 having an amino acid sequence of SEQ ID NOs:640, 641, and 642, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising a VL CDR1, a VL CDR2, and a VL CDR3 having an amino acid sequence of a VL CDR1, a VL CDR2, and a VL CDR3, respectively, of SEQ ID NO:644. In one aspect, provided herein is an antibody that binds TRGV9, comprising a VH having an amino acid sequence of SEQ ID NO:643. In one aspect, provided herein is an antibody that binds TRGV9, comprising a VL having an amino acid sequence of SEQ ID NO:644. In one aspect, provided herein is an antibody that binds TRGV9, comprising a VH having an amino acid sequence of SEQ ID NO:643, and a VL having an amino acid sequence of SEQ ID NO:644. In one aspect, provided herein is an antibody that binds TRGV9, comprising a heavy chain having an amino acid sequence of SEQ ID NO:645. In one aspect, provided herein is an antibody that binds TRGV9, comprising a light chain having an amino acid sequence of SEQ ID NO:646. In one aspect, provided herein is an antibody that binds TRGV9, comprising a heavy chain having an amino acid sequence of SEQ ID NO:645, and a light chain having an amino acid sequence of SEQ ID NO:646. In one aspect, provided herein is an antibody that binds TRGV9, comprising a VH having an amino acid sequence having at least 95% identity to an amino acid sequence of SEQ ID NO:643. In one aspect, provided herein is an antibody that binds TRGV9, comprising a VL having an amino acid sequence having at least 95% identity to an amino acid sequence of SEQ ID NO:644. In one aspect, provided herein is an antibody that binds TRGV9, comprising a VH having an amino acid sequence having at least 95% identity to an amino acid sequence of SEQ ID NO:643, and a VL having an amino acid sequence having at least 95% identity to an amino acid sequence of SEQ ID NO:644. In one aspect, provided herein is an antibody that binds TRGV9, comprising a heavy chain having an amino acid sequence having at least 95% identity to an amino acid sequence of SEQ ID NO:645. In one aspect, provided herein is an antibody that binds TRGV9, comprising a light chain having an amino acid sequence having at least 95% identity to an amino acid sequence of SEQ ID NO:646. In one aspect, provided herein is an antibody that binds TRGV9, comprising a heavy chain having an amino acid sequence having at least 95% identity to an amino acid sequence of SEQ ID NO:645, and a light chain having an amino acid sequence having at least 95% identity to an amino acid sequence of SEQ ID NO:646.

In one aspect, provided herein is an antibody that binds TRGV9, comprising: (i) a VH comprising a VH CDR1, a VH CDR2, and a VH CDR3 having an amino acid sequence of SEQ ID NOs:647, 648, and 649, respectively, and (ii) a VL comprising a VL CDR1, VL CDR2, and VL CDR3 having an amino acid sequence of SEQ ID NOs:650, 651, and 652, respectively. In one aspect, provided herein is an antibody that binds TRGV9, comprising: (i) a VH comprising a VH CDR1, a VH CDR2, and a VH CDR3 having an amino acid sequence of SEQ ID NOs:653, 654, and 655, respectively, and (ii) a VL comprising a VL CDR1, VL CDR2, and VL CDR3 having an amino acid sequence of SEQ ID NOs:656, 657, and 658, respectively. In one aspect, provided herein is an antibody that binds TRGV9, comprising: (i) a VH comprising a VH CDR1, a VH CDR2, and a VH CDR3 having an amino acid sequence of SEQ ID NOs:659, 660, and 661, respectively, and (ii) a VL comprising a VL CDR1, VL CDR2, and VL CDR3 having an amino acid sequence of SEQ ID NOs:662, 663, and 664, respectively. In one aspect, provided herein is an antibody that binds TRGV9, comprising: (i) a VH comprising a VH CDR1, a VH CDR2, and a VH CDR3 having an amino acid sequence of SEQ ID NOs:665, 666, and 667, respectively, and (ii) a VL comprising a VL CDR1, VL CDR2, and VL CDR3 having an amino acid sequence of SEQ ID NOs:668, 669, and 670, respectively. In one aspect, provided herein is an antibody that binds TRGV9, comprising: (i) a VH comprising a VH CDR1, a VH CDR2, and a VH CDR3 having an amino acid sequence of SEQ ID NOs:671, 672, and 673, respectively, and (ii) a VL comprising a VL CDR1, VL CDR2, and VL CDR3 having an amino acid sequence of SEQ ID NOs:674, 675, and 676, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising a VL CDR1, a VL CDR2, and a VL CDR3 having an amino acid sequence of a VL CDR1, a VL CDR2, and a VL CDR3, respectively, of SEQ ID NO:678. In one aspect, provided herein is an antibody that binds TRGV9, comprising a VH having an amino acid sequence of SEQ ID NO:677. In one aspect, provided herein is an antibody that binds TRGV9, comprising a VL having an amino acid sequence of SEQ ID NO:678. In one aspect, provided herein is an antibody that binds TRGV9, comprising a VH having an amino acid sequence of SEQ ID NO:677, and a VL having an amino acid sequence of SEQ ID NO:678. In one aspect, provided herein is an antibody that binds TRGV9, comprising a heavy chain having an amino acid sequence of SEQ ID NO:679. In one aspect, provided herein is an antibody that binds TRGV9, comprising a light chain having an amino acid sequence of SEQ ID NO:680. In one aspect, provided herein is an antibody that binds TRGV9, comprising a heavy chain having an amino acid sequence of SEQ ID NO:679, and a light chain having an amino acid sequence of SEQ ID NO:680. In one aspect, provided herein is an antibody that binds TRGV9, comprising a VH having an amino acid sequence having at least 95% identity to an amino acid sequence of SEQ ID NO:677. In one aspect, provided herein is an antibody that binds TRGV9, comprising a VL having an amino acid sequence having at least 95% identity to an amino acid sequence of SEQ ID NO:678. In one aspect, provided herein is an antibody that binds TRGV9, comprising a VH having an amino acid sequence having at least 95% identity to an amino acid sequence of SEQ ID NO:677, and a VL having an amino acid sequence having at least 95% identity to an amino acid sequence of SEQ ID NO:678. In one aspect, provided herein is an antibody that binds TRGV9, comprising a heavy chain having an amino acid sequence having at least 95% identity to an amino acid sequence of SEQ ID NO:679. In one aspect, provided herein is an antibody that binds TRGV9, comprising a light chain having an amino acid sequence having at least 95% identity to an amino acid sequence of SEQ ID NO:680. In one aspect, provided herein is an antibody that binds TRGV9, comprising a heavy chain having an amino acid sequence having at least 95% identity to an amino acid sequence of SEQ ID NO:679, and a light chain having an amino acid sequence having at least 95% identity to an amino acid sequence of SEQ ID NO:680.

In one aspect, provided herein is an antibody that binds TRGV9, comprising: (i) a VH comprising a VH CDR1, a VH CDR2, and a VH CDR3 having an amino acid sequence of SEQ ID NOs:681, 682, and 683, respectively, and (ii) a VL comprising a VL CDR1, VL CDR2, and VL CDR3 having an amino acid sequence of SEQ ID NOs:684, 685, and 686, respectively. In one aspect, provided herein is an antibody that binds TRGV9, comprising: (i) a VH comprising a VH CDR1, a VH CDR2, and a VH CDR3 having an amino acid sequence of SEQ ID NOs:687, 688, and 689, respectively, and (ii) a VL comprising a VL CDR1, VL CDR2, and VL CDR3 having an amino acid sequence of SEQ ID NOs:690, 691, and 692, respectively. In one aspect, provided herein is an antibody that binds TRGV9, comprising: (i) a VH comprising a VH CDR1, a VH CDR2, and a VH CDR3 having an amino acid sequence of SEQ ID NOs:693, 694, and 695, respectively, and (ii) a VL comprising a VL CDR1, VL CDR2, and VL CDR3 having an amino acid sequence of SEQ ID NOs:696, 697, and 698, respectively. In one aspect, provided herein is an antibody that binds TRGV9, comprising: (i) a VH comprising a VH CDR1, a VH CDR2, and a VH CDR3 having an amino acid sequence of SEQ ID NOs:699, 700, and 701, respectively, and (ii) a VL comprising a VL CDR1, VL CDR2, and VL CDR3 having an amino acid sequence of SEQ ID NOs:702, 703, and 704, respectively. In one aspect, provided herein is an antibody that binds TRGV9, comprising: (i) a VH comprising a VH CDR1, a VH CDR2, and a VH CDR3 having an amino acid sequence of SEQ ID NOs:705, 706, and 707, respectively, and (ii) a VL comprising a VL CDR1, VL CDR2, and VL CDR3 having an amino acid sequence of SEQ ID NOs:708, 709, and 710, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising a VL CDR1, a VL CDR2, and a VL CDR3 having an amino acid sequence of a VL CDR1, a VL CDR2, and a VL CDR3, respectively, of SEQ ID NO:712. In one aspect, provided herein is an antibody that binds TRGV9, comprising a VH having an amino acid sequence of SEQ ID NO:711. In one aspect, provided herein is an antibody that binds TRGV9, comprising a VL having an amino acid sequence of SEQ ID NO:712. In one aspect, provided herein is an antibody that binds TRGV9, comprising a VH having an amino acid sequence of SEQ ID NO:711, and a VL having an amino acid sequence of SEQ ID NO:712. In one aspect, provided herein is an antibody that binds TRGV9, comprising a heavy chain having an amino acid sequence of SEQ ID NO:713. In one aspect, provided herein is an antibody that binds TRGV9, comprising a light chain having an amino acid sequence of SEQ ID NO:714. In one aspect, provided herein is an antibody that binds TRGV9, comprising a heavy chain having an amino acid sequence of SEQ ID NO:713, and a light chain having an amino acid sequence of SEQ ID NO:714. In one aspect, provided herein is an antibody that binds TRGV9, comprising a VH having an amino acid sequence having at least 95% identity to an amino acid sequence of SEQ ID NO:711. In one aspect, provided herein is an antibody that binds TRGV9, comprising a VL having an amino acid sequence having at least 95% identity to an amino acid sequence of SEQ ID NO:712. In one aspect, provided herein is an antibody that binds TRGV9, comprising a VH having an amino acid sequence having at least 95% identity to an amino acid sequence of SEQ ID NO:711, and a VL having an amino acid sequence having at least 95% identity to an amino acid sequence of SEQ ID NO:712. In one aspect, provided herein is an antibody that binds TRGV9, comprising a heavy chain having an amino acid sequence having at least 95% identity to an amino acid sequence of SEQ ID NO:713. In one aspect, provided herein is an antibody that binds TRGV9, comprising a light chain having an amino acid sequence having at least 95% identity to an amino acid sequence of SEQ ID NO:714. In one aspect, provided herein is an antibody that binds TRGV9, comprising a heavy chain having an amino acid sequence having at least 95% identity to an amino acid sequence of SEQ ID NO:713, and a light chain having an amino acid sequence having at least 95% identity to an amino acid sequence of SEQ ID NO:714.

In one aspect, provided herein is an antibody that binds TRGV9, comprising: (i) a VH comprising a VH CDR1, a VH CDR2, and a VH CDR3 having an amino acid sequence of SEQ ID NOs:715, 716, and 717, respectively, and (ii) a VL comprising a VL CDR1, VL CDR2, and VL CDR3 having an amino acid sequence of SEQ ID NOs:718, 719, and 720, respectively. In one aspect, provided herein is an antibody that binds TRGV9, comprising: (i) a VH comprising a VH CDR1, a VH CDR2, and a VH CDR3 having an amino acid sequence of SEQ ID NOs:721, 722, and 723, respectively, and (ii) a VL comprising a VL CDR1, VL CDR2, and VL CDR3 having an amino acid sequence of SEQ ID NOs:724, 725, and 726, respectively. In one aspect, provided herein is an antibody that binds TRGV9, comprising: (i) a VH comprising a VH CDR1, a VH CDR2, and a VH CDR3 having an amino acid sequence of SEQ ID NOs:727, 728, and 729, respectively, and (ii) a VL comprising a VL CDR1, VL CDR2, and VL CDR3 having an amino acid sequence of SEQ ID NOs:730, 731, and 732, respectively. In one aspect, provided herein is an antibody that binds TRGV9, comprising: (i) a VH comprising a VH CDR1, a VH CDR2, and a VH CDR3 having an amino acid sequence of SEQ ID NOs:733, 734, and 735, respectively, and (ii) a VL comprising a VL CDR1, VL CDR2, and VL CDR3 having an amino acid sequence of SEQ ID NOs:736, 737, and 738, respectively. In one aspect, provided herein is an antibody that binds TRGV9, comprising: (i) a VH comprising a VH CDR1, a VH CDR2, and a VH CDR3 having an amino acid sequence of SEQ ID NOs:739, 740, and 741, respectively, and (ii) a VL comprising a VL CDR1, VL CDR2, and VL CDR3 having an amino acid sequence of SEQ ID NOs:742, 743, and 744, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising a VL CDR1, a VL CDR2, and a VL CDR3 having an amino acid sequence of a VL CDR1, a VL CDR2, and a VL CDR3, respectively, of SEQ ID NO:746. In one aspect, provided herein is an antibody that binds TRGV9, comprising a VH having an amino acid sequence of SEQ ID NO:745. In one aspect, provided herein is an antibody that binds TRGV9, comprising a VL having an amino acid sequence of SEQ ID NO:746. In one aspect, provided herein is an antibody that binds TRGV9, comprising a VH having an amino acid sequence of SEQ ID NO:745, and a VL having an amino acid sequence of SEQ ID NO:746. In one aspect, provided herein is an antibody that binds TRGV9, comprising a heavy chain having an amino acid sequence of SEQ ID NO:747. In one aspect, provided herein is an antibody that binds TRGV9, comprising a light chain having an amino acid sequence of SEQ ID NO:748. In one aspect, provided herein is an antibody that binds TRGV9, comprising a heavy chain having an amino acid sequence of SEQ ID NO:747, and a light chain having an amino acid sequence of SEQ ID NO:748. In one aspect, provided herein is an antibody that binds TRGV9, comprising a VH having an amino acid sequence having at least 95% identity to an amino acid sequence of SEQ ID NO:745. In one aspect, provided herein is an antibody that binds TRGV9, comprising a VL having an amino acid sequence having at least 95% identity to an amino acid sequence of SEQ ID NO:746. In one aspect, provided herein is an antibody that binds TRGV9, comprising a VH having an amino acid sequence having at least 95% identity to an amino acid sequence of SEQ ID NO:745, and a VL having an amino acid sequence having at least 95% identity to an amino acid sequence of SEQ ID NO:746. In one aspect, provided herein is an antibody that binds TRGV9, comprising a heavy chain having an amino acid sequence having at least 95% identity to an amino acid sequence of SEQ ID NO:747. In one aspect, provided herein is an antibody that binds TRGV9, comprising a light chain having an amino acid sequence having at least 95% identity to an amino acid sequence of SEQ ID NO:748. In one aspect, provided herein is an antibody that binds TRGV9, comprising a heavy chain having an amino acid sequence having at least 95% identity to an amino acid sequence of SEQ ID NO:747, and a light chain having an amino acid sequence having at least 95% identity to an amino acid sequence of SEQ ID NO:748.

In one aspect, provided herein is an antibody that binds TRGV9, comprising: (i) a VH comprising a VH CDR1, a VH CDR2, and a VH CDR3 having an amino acid sequence of SEQ ID NOs:749, 750, and 751, respectively, and (ii) a VL comprising a VL CDR1, VL CDR2, and VL CDR3 having an amino acid sequence of SEQ ID NOs:752, 753, and 754, respectively. In one aspect, provided herein is an antibody that binds TRGV9, comprising: (i) a VH comprising a VH CDR1, a VH CDR2, and a VH CDR3 having an amino acid sequence of SEQ ID NOs:755, 756, and 757, respectively, and (ii) a VL comprising a VL CDR1, VL CDR2, and VL CDR3 having an amino acid sequence of SEQ ID NOs:758, 759, and 760, respectively. In one aspect, provided herein is an antibody that binds TRGV9, comprising: (i) a VH comprising a VH CDR1, a VH CDR2, and a VH CDR3 having an amino acid sequence of SEQ ID NOs:761, 762, and 763, respectively, and (ii) a VL comprising a VL CDR1, VL CDR2, and VL CDR3 having an amino acid sequence of SEQ ID NOs:764, 765, and 766, respectively. In one aspect, provided herein is an antibody that binds TRGV9, comprising: (i) a VH comprising a VH CDR1, a VH CDR2, and a VH CDR3 having an amino acid sequence of SEQ ID NOs:767, 768, and 769, respectively, and (ii) a VL comprising a VL CDR1, VL CDR2, and VL CDR3 having an amino acid sequence of SEQ ID NOs:770, 771, and 772, respectively. In one aspect, provided herein is an antibody that binds TRGV9, comprising: (i) a VH comprising a VH CDR1, a VH CDR2, and a VH CDR3 having an amino acid sequence of SEQ ID NOs:773, 774, and 775, respectively, and (ii) a VL comprising a VL CDR1, VL CDR2, and VL CDR3 having an amino acid sequence of SEQ ID NOs:776, 777, and 778, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising a VL CDR1, a VL CDR2, and a VL CDR3 having an amino acid sequence of a VL CDR1, a VL CDR2, and a VL CDR3, respectively, of SEQ ID NO:780. In one aspect, provided herein is an antibody that binds TRGV9, comprising a VH having an amino acid sequence of SEQ ID NO:779. In one aspect, provided herein is an antibody that binds TRGV9, comprising a VL having an amino acid sequence of SEQ ID NO:780. In one aspect, provided herein is an antibody that binds TRGV9, comprising a VH having an amino acid sequence of SEQ ID NO:779, and a VL having an amino acid sequence of SEQ ID NO:780. In one aspect, provided herein is an antibody that binds TRGV9, comprising a heavy chain having an amino acid sequence of SEQ ID NO:781. In one aspect, provided herein is an antibody that binds TRGV9, comprising a light chain having an amino acid sequence of SEQ ID NO:782. In one aspect, provided herein is an antibody that binds TRGV9, comprising a heavy chain having an amino acid sequence of SEQ ID NO:781, and a light chain having an amino acid sequence of SEQ ID NO:782. In one aspect, provided herein is an antibody that binds TRGV9, comprising a VH having an amino acid sequence having at least 95% identity to an amino acid sequence of SEQ ID NO:779. In one aspect, provided herein is an antibody that binds TRGV9, comprising a VL having an amino acid sequence having at least 95% identity to an amino acid sequence of SEQ ID NO:780. In one aspect, provided herein is an antibody that binds TRGV9, comprising a VH having an amino acid sequence having at least 95% identity to an amino acid sequence of SEQ ID NO:779, and a VL having an amino acid sequence having at least 95% identity to an amino acid sequence of SEQ ID NO:780. In one aspect, provided herein is an antibody that binds TRGV9, comprising a heavy chain having an amino acid sequence having at least 95% identity to an amino acid sequence of SEQ ID NO:781. In one aspect, provided herein is an antibody that binds TRGV9, comprising a light chain having an amino acid sequence having at least 95% identity to an amino acid sequence of SEQ ID NO:782. In one aspect, provided herein is an antibody that binds TRGV9, comprising a heavy chain having an amino acid sequence having at least 95% identity to an amino acid sequence of SEQ ID NO:781, and a light chain having an amino acid sequence having at least 95% identity to an amino acid sequence of SEQ ID NO:782.

In one aspect, provided herein is an antibody that binds TRGV9, comprising: (i) a VH comprising a VH CDR1, a VH CDR2, and a VH CDR3 having an amino acid sequence of SEQ ID NOs:783, 784, and 785, respectively, and (ii) a VL comprising a VL CDR1, VL CDR2, and VL CDR3 having an amino acid sequence of SEQ ID NOs:786, 787, and 788, respectively. In one aspect, provided herein is an antibody that binds TRGV9, comprising: (i) a VH comprising a VH CDR1, a VH CDR2, and a VH CDR3 having an amino acid sequence of SEQ ID NOs:789, 790, and 791, respectively, and (ii) a VL comprising a VL CDR1, VL CDR2, and VL CDR3 having an amino acid sequence of SEQ ID NOs:792, 793, and 794, respectively. In one aspect, provided herein is an antibody that binds TRGV9, comprising: (i) a VH comprising a VH CDR1, a VH CDR2, and a VH CDR3 having an amino acid sequence of SEQ ID NOs:795, 796, and 797, respectively, and (ii) a VL comprising a VL CDR1, VL CDR2, and VL CDR3 having an amino acid sequence of SEQ ID NOs:798, 799, and 800, respectively. In one aspect, provided herein is an antibody that binds TRGV9, comprising: (i) a VH comprising a VH CDR1, a VH CDR2, and a VH CDR3 having an amino acid sequence of SEQ ID NOs:801, 802, and 803, respectively, and (ii) a VL comprising a VL CDR1, VL CDR2, and VL CDR3 having an amino acid sequence of SEQ ID NOs:804, 805, and 806, respectively. In one aspect, provided herein is an antibody that binds TRGV9, comprising: (i) a VH comprising a VH CDR1, a VH CDR2, and a VH CDR3 having an amino acid sequence of SEQ ID NOs:807, 808, and 809, respectively, and (ii) a VL comprising a VL CDR1, VL CDR2, and VL CDR3 having an amino acid sequence of SEQ ID NOs:810, 811, and 812, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising a VL CDR1, a VL CDR2, and a VL CDR3 having an amino acid sequence of a VL CDR1, a VL CDR2, and a VL CDR3, respectively, of SEQ ID NO:814. In one aspect, provided herein is an antibody that binds TRGV9, comprising a VH having an amino acid sequence of SEQ ID NO:813. In one aspect, provided herein is an antibody that binds TRGV9, comprising a VL having an amino acid sequence of SEQ ID NO:814. In one aspect, provided herein is an antibody that binds TRGV9, comprising a VH having an amino acid sequence of SEQ ID NO:813, and a VL having an amino acid sequence of SEQ ID NO:814. In one aspect, provided herein is an antibody that binds TRGV9, comprising a heavy chain having an amino acid sequence of SEQ ID NO:815. In one aspect, provided herein is an antibody that binds TRGV9, comprising a light chain having an amino acid sequence of SEQ ID NO:816. In one aspect, provided herein is an antibody that binds TRGV9, comprising a heavy chain having an amino acid sequence of SEQ ID NO:815, and a light chain having an amino acid sequence of SEQ ID NO:816. In one aspect, provided herein is an antibody that binds TRGV9, comprising a VH having an amino acid sequence having at least 95% identity to an amino acid sequence of SEQ ID NO:813. In one aspect, provided herein is an antibody that binds TRGV9, comprising a VL having an amino acid sequence having at least 95% identity to an amino acid sequence of SEQ ID NO:814. In one aspect, provided herein is an antibody that binds TRGV9, comprising a VH having an amino acid sequence having at least 95% identity to an amino acid sequence of SEQ ID NO:813, and a VL having an amino acid sequence having at least 95% identity to an amino acid sequence of SEQ ID NO:814. In one aspect, provided herein is an antibody that binds TRGV9, comprising a heavy chain having an amino acid sequence having at least 95% identity to an amino acid sequence of SEQ ID NO:815. In one aspect, provided herein is an antibody that binds TRGV9, comprising a light chain having an amino acid sequence having at least 95% identity to an amino acid sequence of SEQ ID NO:816. In one aspect, provided herein is an antibody that binds TRGV9, comprising a heavy chain having an amino acid sequence having at least 95% identity to an amino acid sequence of SEQ ID NO:815, and a light chain having an amino acid sequence having at least 95% identity to an amino acid sequence of SEQ ID NO:816.

In one aspect, provided herein is an antibody that binds TRGV9, comprising: (i) a VH comprising a VH CDR1, a VH CDR2, and a VH CDR3 having an amino acid sequence of SEQ ID NOs:817, 818, and 819, respectively, and (ii) a VL comprising a VL CDR1, VL CDR2, and VL CDR3 having an amino acid sequence of SEQ ID NOs:820, 821, and 822, respectively. In one aspect, provided herein is an antibody that binds TRGV9, comprising: (i) a VH comprising a VH CDR1, a VH CDR2, and a VH CDR3 having an amino acid sequence of SEQ ID NOs:823, 824, and 825, respectively, and (ii) a VL comprising a VL CDR1, VL CDR2, and VL CDR3 having an amino acid sequence of SEQ ID NOs:826, 827, and 828, respectively. In one aspect, provided herein is an antibody that binds TRGV9, comprising: (i) a VH comprising a VH CDR1, a VH CDR2, and a VH CDR3 having an amino acid sequence of SEQ ID NOs:829, 830, and 831, respectively, and (ii) a VL comprising a VL CDR1, VL CDR2, and VL CDR3 having an amino acid sequence of SEQ ID NOs:832, 833, and 834, respectively. In one aspect, provided herein is an antibody that binds TRGV9, comprising: (i) a VH comprising a VH CDR1, a VH CDR2, and a VH CDR3 having an amino acid sequence of SEQ ID NOs:835, 836, and 837, respectively, and (ii) a VL comprising a VL CDR1, VL CDR2, and VL CDR3 having an amino acid sequence of SEQ ID NOs:838, 839, and 840, respectively. In one aspect, provided herein is an antibody that binds TRGV9, comprising: (i) a VH comprising a VH CDR1, a VH CDR2, and a VH CDR3 having an amino acid sequence of SEQ ID NOs:841, 842, and 843, respectively, and (ii) a VL comprising a VL CDR1, VL CDR2, and VL CDR3 having an amino acid sequence of SEQ ID NOs:844, 845, and 846, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:847; and (ii) a VL comprising a VL CDR1, a VL CDR2, and a VL CDR3 having an amino acid sequence of a VL CDR1, a VL CDR2, and a VL CDR3, respectively, of SEQ ID NO:848. In one aspect, provided herein is an antibody that binds TRGV9, comprising a VH having an amino acid sequence of SEQ ID NO:847. In one aspect, provided herein is an antibody that binds TRGV9, comprising a VL having an amino acid sequence of SEQ ID NO:848. In one aspect, provided herein is an antibody that binds TRGV9, comprising a VH having an amino acid sequence of SEQ ID NO:847, and a VL having an amino acid sequence of SEQ ID NO:848. In one aspect, provided herein is an antibody that binds TRGV9, comprising a heavy chain having an amino acid sequence of SEQ ID NO:849. In one aspect, provided herein is an antibody that binds TRGV9, comprising a light chain having an amino acid sequence of SEQ ID NO:850. In one aspect, provided herein is an antibody that binds TRGV9, comprising a heavy chain having an amino acid sequence of SEQ ID NO:849, and a light chain having an amino acid sequence of SEQ ID NO:850. In one aspect, provided herein is an antibody that binds TRGV9, comprising a VH having an amino acid sequence having at least 95% identity to an amino acid sequence of SEQ ID NO:847. In one aspect, provided herein is an antibody that binds TRGV9, comprising a VL having an amino acid sequence having at least 95% identity to an amino acid sequence of SEQ ID NO:848. In one aspect, provided herein is an antibody that binds TRGV9, comprising a VH having an amino acid sequence having at least 95% identity to an amino acid sequence of SEQ ID NO:847, and a VL having an amino acid sequence having at least 95% identity to an amino acid sequence of SEQ ID NO:848. In one aspect, provided herein is an antibody that binds TRGV9, comprising a heavy chain having an amino acid sequence having at least 95% identity to an amino acid sequence of SEQ ID NO:849. In one aspect, provided herein is an antibody that binds TRGV9, comprising a light chain having an amino acid sequence having at least 95% identity to an amino acid sequence of SEQ ID NO:850. In one aspect, provided herein is an antibody that binds TRGV9, comprising a heavy chain having an amino acid sequence having at least 95% identity to an amino acid sequence of SEQ ID NO:849, and a light chain having an amino acid sequence having at least 95% identity to an amino acid sequence of SEQ ID NO:850.

In one aspect, provided herein is an antibody that binds TRGV9, comprising: (i) a VH comprising a VH CDR1, a VH CDR2, and a VH CDR3 having an amino acid sequence of SEQ ID NOs:851, 852, and 853, respectively, and (ii) a VL comprising a VL CDR1, VL CDR2, and VL CDR3 having an amino acid sequence of SEQ ID NOs:854, 855, and 856, respectively. In one aspect, provided herein is an antibody that binds TRGV9, comprising: (i) a VH comprising a VH CDR1, a VH CDR2, and a VH CDR3 having an amino acid sequence of SEQ ID NOs:857, 858, and 859, respectively, and (ii) a VL comprising a VL CDR1, VL CDR2, and VL CDR3 having an amino acid sequence of SEQ ID NOs:860, 861, and 862, respectively. In one aspect, provided herein is an antibody that binds TRGV9, comprising: (i) a VH comprising a VH CDR1, a VH CDR2, and a VH CDR3 having an amino acid sequence of SEQ ID NOs:863, 864, and 865, respectively, and (ii) a VL comprising a VL CDR1, VL CDR2, and VL CDR3 having an amino acid sequence of SEQ ID NOs:866, 867, and 868, respectively. In one aspect, provided herein is an antibody that binds TRGV9, comprising: (i) a VH comprising a VH CDR1, a VH CDR2, and a VH CDR3 having an amino acid sequence of SEQ ID NOs:869, 870, and 871, respectively, and (ii) a VL comprising a VL CDR1, VL CDR2, and VL CDR3 having an amino acid sequence of SEQ ID NOs:872, 873, and 874, respectively. In one aspect, provided herein is an antibody that binds TRGV9, comprising: (i) a VH comprising a VH CDR1, a VH CDR2, and a VH CDR3 having an amino acid sequence of SEQ ID NOs:875, 876, and 877, respectively, and (ii) a VL comprising a VL CDR1, VL CDR2, and VL CDR3 having an amino acid sequence of SEQ ID NOs:878, 879, and 880, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:881; and (ii) a VL comprising a VL CDR1, a VL CDR2, and a VL CDR3 having an amino acid sequence of a VL CDR1, a VL CDR2, and a VL CDR3, respectively, of SEQ ID NO:882. In one aspect, provided herein is an antibody that binds TRGV9, comprising a VH having an amino acid sequence of SEQ ID NO:881. In one aspect, provided herein is an antibody that binds TRGV9, comprising a VL having an amino acid sequence of SEQ ID NO:882. In one aspect, provided herein is an antibody that binds TRGV9, comprising a VH having an amino acid sequence of SEQ ID NO:881, and a VL having an amino acid sequence of SEQ ID NO:882. In one aspect, provided herein is an antibody that binds TRGV9, comprising a heavy chain having an amino acid sequence of SEQ ID NO:883. In one aspect, provided herein is an antibody that binds TRGV9, comprising a light chain having an amino acid sequence of SEQ ID NO:884. In one aspect, provided herein is an antibody that binds TRGV9, comprising a heavy chain having an amino acid sequence of SEQ ID NO:883, and a light chain having an amino acid sequence of SEQ ID NO:884. In one aspect, provided herein is an antibody that binds TRGV9, comprising a VH having an amino acid sequence having at least 95% identity to an amino acid sequence of SEQ ID NO:881. In one aspect, provided herein is an antibody that binds TRGV9, comprising a VL having an amino acid sequence having at least 95% identity to an amino acid sequence of SEQ ID NO:882. In one aspect, provided herein is an antibody that binds TRGV9, comprising a VH having an amino acid sequence having at least 95% identity to an amino acid sequence of SEQ ID NO:881, and a VL having an amino acid sequence having at least 95% identity to an amino acid sequence of SEQ ID NO:882. In one aspect, provided herein is an antibody that binds TRGV9, comprising a heavy chain having an amino acid sequence having at least 95% identity to an amino acid sequence of SEQ ID NO:883. In one aspect, provided herein is an antibody that binds TRGV9, comprising a light chain having an amino acid sequence having at least 95% identity to an amino acid sequence of SEQ ID NO:884. In one aspect, provided herein is an antibody that binds TRGV9, comprising a heavy chain having an amino acid sequence having at least 95% identity to an amino acid sequence of SEQ ID NO:883, and a light chain having an amino acid sequence having at least 95% identity to an amino acid sequence of SEQ ID NO:884.

In one aspect, provided herein is an antibody that binds TRGV9, comprising: (i) a VH comprising a VH CDR1, a VH CDR2, and a VH CDR3 having an amino acid sequence of SEQ ID NOs:885, 886, and 887, respectively, and (ii) a VL comprising a VL CDR1, VL CDR2, and VL CDR3 having an amino acid sequence of SEQ ID NOs:888, 889, and 890, respectively. In one aspect, provided herein is an antibody that binds TRGV9, comprising: (i) a VH comprising a VH CDR1, a VH CDR2, and a VH CDR3 having an amino acid sequence of SEQ ID NOs:891, 892, and 893, respectively, and (ii) a VL comprising a VL CDR1, VL CDR2, and VL CDR3 having an amino acid sequence of SEQ ID NOs:894, 895, and 896, respectively. In one aspect, provided herein is an antibody that binds TRGV9, comprising: (i) a VH comprising a VH CDR1, a VH CDR2, and a VH CDR3 having an amino acid sequence of SEQ ID NOs:897, 898, and 899, respectively, and (ii) a VL comprising a VL CDR1, VL CDR2, and VL CDR3 having an amino acid sequence of SEQ ID NOs:900, 901, and 902, respectively. In one aspect, provided herein is an antibody that binds TRGV9, comprising: (i) a VH comprising a VH CDR1, a VH CDR2, and a VH CDR3 having an amino acid sequence of SEQ ID NOs:903, 904, and 905, respectively, and (ii) a VL comprising a VL CDR1, VL CDR2, and VL CDR3 having an amino acid sequence of SEQ ID NOs:906, 907, and 908, respectively. In one aspect, provided herein is an antibody that binds TRGV9, comprising: (i) a VH comprising a VH CDR1, a VH CDR2, and a VH CDR3 having an amino acid sequence of SEQ ID NOs:909, 910, and 911, respectively, and (ii) a VL comprising a VL CDR1, VL CDR2, and VL CDR3 having an amino acid sequence of SEQ ID NOs:912, 913, and 914, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:915; and (ii) a VL comprising a VL CDR1, a VL CDR2, and a VL CDR3 having an amino acid sequence of a VL CDR1, a VL CDR2, and a VL CDR3, respectively, of SEQ ID NO:916. In one aspect, provided herein is an antibody that binds TRGV9, comprising a VH having an amino acid sequence of SEQ ID NO:915. In one aspect, provided herein is an antibody that binds TRGV9, comprising a VL having an amino acid sequence of SEQ ID NO:916. In one aspect, provided herein is an antibody that binds TRGV9, comprising a VH having an amino acid sequence of SEQ ID NO:915, and a VL having an amino acid sequence of SEQ ID NO:916. In one aspect, provided herein is an antibody that binds TRGV9, comprising a heavy chain having an amino acid sequence of SEQ ID NO:917. In one aspect, provided herein is an antibody that binds TRGV9, comprising a light chain having an amino acid sequence of SEQ ID NO:918. In one aspect, provided herein is an antibody that binds TRGV9, comprising a heavy chain having an amino acid sequence of SEQ ID NO:917, and a light chain having an amino acid sequence of SEQ ID NO:918. In one aspect, provided herein is an antibody that binds TRGV9, comprising a VH having an amino acid sequence having at least 95% identity to an amino acid sequence of SEQ ID NO:915. In one aspect, provided herein is an antibody that binds TRGV9, comprising a VL having an amino acid sequence having at least 95% identity to an amino acid sequence of SEQ ID NO:916. In one aspect, provided herein is an antibody that binds TRGV9, comprising a VH having an amino acid sequence having at least 95% identity to an amino acid sequence of SEQ ID NO:915, and a VL having an amino acid sequence having at least 95% identity to an amino acid sequence of SEQ ID NO:916. In one aspect, provided herein is an antibody that binds TRGV9, comprising a heavy chain having an amino acid sequence having at least 95% identity to an amino acid sequence of SEQ ID NO:917. In one aspect, provided herein is an antibody that binds TRGV9, comprising a light chain having an amino acid sequence having at least 95% identity to an amino acid sequence of SEQ ID NO:918. In one aspect, provided herein is an antibody that binds TRGV9, comprising a heavy chain having an amino acid sequence having at least 95% identity to an amino acid sequence of SEQ ID NO:917, and a light chain having an amino acid sequence having at least 95% identity to an amino acid sequence of SEQ ID NO:918.

In one aspect, provided herein is an antibody that binds TRGV9, comprising: (i) a VH comprising a VH CDR1, a VH CDR2, and a VH CDR3 having an amino acid sequence of SEQ ID NOs:919, 920, and 921, respectively, and (ii) a VL comprising a VL CDR1, VL CDR2, and VL CDR3 having an amino acid sequence of SEQ ID NOs:922, 923, and 924, respectively. In one aspect, provided herein is an antibody that binds TRGV9, comprising: (i) a VH comprising a VH CDR1, a VH CDR2, and a VH CDR3 having an amino acid sequence of SEQ ID NOs:925, 926, and 927, respectively, and (ii) a VL comprising a VL CDR1, VL CDR2, and VL CDR3 having an amino acid sequence of SEQ ID NOs:928, 929, and 930, respectively. In one aspect, provided herein is an antibody that binds TRGV9, comprising: (i) a VH comprising a VH CDR1, a VH CDR2, and a VH CDR3 having an amino acid sequence of SEQ ID NOs:931, 932, and 933, respectively, and (ii) a VL comprising a VL CDR1, VL CDR2, and VL CDR3 having an amino acid sequence of SEQ ID NOs:934, 935, and 936, respectively. In one aspect, provided herein is an antibody that binds TRGV9, comprising: (i) a VH comprising a VH CDR1, a VH CDR2, and a VH CDR3 having an amino acid sequence of SEQ ID NOs:937, 938, and 939, respectively, and (ii) a VL comprising a VL CDR1, VL CDR2, and VL CDR3 having an amino acid sequence of SEQ ID NOs:940, 941, and 942, respectively. In one aspect, provided herein is an antibody that binds TRGV9, comprising: (i) a VH comprising a VH CDR1, a VH CDR2, and a VH CDR3 having an amino acid sequence of SEQ ID NOs:943, 944, and 945, respectively, and (ii) a VL comprising a VL CDR1, VL CDR2, and VL CDR3 having an amino acid sequence of SEQ ID NOs:946, 947, and 948, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:949; and (ii) a VL comprising a VL CDR1, a VL CDR2, and a VL CDR3 having an amino acid sequence of a VL CDR1, a VL CDR2, and a VL CDR3, respectively, of SEQ ID NO:950. In one aspect, provided herein is an antibody that binds TRGV9, comprising a VH having an amino acid sequence of SEQ ID NO:949. In one aspect, provided herein is an antibody that binds TRGV9, comprising a VL having an amino acid sequence of SEQ ID NO:950. In one aspect, provided herein is an antibody that binds TRGV9, comprising a VH having an amino acid sequence of SEQ ID NO:949, and a VL having an amino acid sequence of SEQ ID NO:950. In one aspect, provided herein is an antibody that binds TRGV9, comprising a heavy chain having an amino acid sequence of SEQ ID NO:951. In one aspect, provided herein is an antibody that binds TRGV9, comprising a light chain having an amino acid sequence of SEQ ID NO:952. In one aspect, provided herein is an antibody that binds TRGV9, comprising a heavy chain having an amino acid sequence of SEQ ID NO:951, and a light chain having an amino acid sequence of SEQ ID NO:952. In one aspect, provided herein is an antibody that binds TRGV9, comprising a VH having an amino acid sequence having at least 95% identity to an amino acid sequence of SEQ ID NO:949. In one aspect, provided herein is an antibody that binds TRGV9, comprising a VL having an amino acid sequence having at least 95% identity to an amino acid sequence of SEQ ID NO:950. In one aspect, provided herein is an antibody that binds TRGV9, comprising a VH having an amino acid sequence having at least 95% identity to an amino acid sequence of SEQ ID NO:949, and a VL having an amino acid sequence having at least 95% identity to an amino acid sequence of SEQ ID NO:950. In one aspect, provided herein is an antibody that binds TRGV9, comprising a heavy chain having an amino acid sequence having at least 95% identity to an amino acid sequence of SEQ ID NO:951. In one aspect, provided herein is an antibody that binds TRGV9, comprising a light chain having an amino acid sequence having at least 95% identity to an amino acid sequence of SEQ ID NO:952. In one aspect, provided herein is an antibody that binds TRGV9, comprising a heavy chain having an amino acid sequence having at least 95% identity to an amino acid sequence of SEQ ID NO:951, and a light chain having an amino acid sequence having at least 95% identity to an amino acid sequence of SEQ ID NO:952.

In one aspect, provided herein is an antibody that binds TRGV9, comprising: (i) a VH comprising a VH CDR1, a VH CDR2, and a VH CDR3 having an amino acid sequence of SEQ ID NOs:953, 954, and 955, respectively, and (ii) a VL comprising a VL CDR1, VL CDR2, and VL CDR3 having an amino acid sequence of SEQ ID NOs:956, 957, and 958, respectively. In one aspect, provided herein is an antibody that binds TRGV9, comprising: (i) a VH comprising a VH CDR1, a VH CDR2, and a VH CDR3 having an amino acid sequence of SEQ ID NOs:959, 960, and 961, respectively, and (ii) a VL comprising a VL CDR1, VL CDR2, and VL CDR3 having an amino acid sequence of SEQ ID NOs:962, 963, and 964, respectively. In one aspect, provided herein is an antibody that binds TRGV9, comprising: (i) a VH comprising a VH CDR1, a VH CDR2, and a VH CDR3 having an amino acid sequence of SEQ ID NOs:965, 966, and 967, respectively, and (ii) a VL comprising a VL CDR1, VL CDR2, and VL CDR3 having an amino acid sequence of SEQ ID NOs:968, 969, and 970, respectively. In one aspect, provided herein is an antibody that binds TRGV9, comprising: (i) a VH comprising a VH CDR1, a VH CDR2, and a VH CDR3 having an amino acid sequence of SEQ ID NOs:971, 972, and 973, respectively, and (ii) a VL comprising a VL CDR1, VL CDR2, and VL CDR3 having an amino acid sequence of SEQ ID NOs:974, 975, and 976, respectively. In one aspect, provided herein is an antibody that binds TRGV9, comprising: (i) a VH comprising a VH CDR1, a VH CDR2, and a VH CDR3 having an amino acid sequence of SEQ ID NOs:977, 978, and 979, respectively, and (ii) a VL comprising a VL CDR1, VL CDR2, and VL CDR3 having an amino acid sequence of SEQ ID NOs:980, 981, and 982, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:983; and (ii) a VL comprising a VL CDR1, a VL CDR2, and a VL CDR3 having an amino acid sequence of a VL CDR1, a VL CDR2, and a VL CDR3, respectively, of SEQ ID NO:984. In one aspect, provided herein is an antibody that binds TRGV9, comprising a VH having an amino acid sequence of SEQ ID NO:983. In one aspect, provided herein is an antibody that binds TRGV9, comprising a VL having an amino acid sequence of SEQ ID NO:984. In one aspect, provided herein is an antibody that binds TRGV9, comprising a VH having an amino acid sequence of SEQ ID NO:983, and a VL having an amino acid sequence of SEQ ID NO:984. In one aspect, provided herein is an antibody that binds TRGV9, comprising a heavy chain having an amino acid sequence of SEQ ID NO:985. In one aspect, provided herein is an antibody that binds TRGV9, comprising a light chain having an amino acid sequence of SEQ ID NO:986. In one aspect, provided herein is an antibody that binds TRGV9, comprising a heavy chain having an amino acid sequence of SEQ ID NO:985, and a light chain having an amino acid sequence of SEQ ID NO:986. In one aspect, provided herein is an antibody that binds TRGV9, comprising a VH having an amino acid sequence having at least 95% identity to an amino acid sequence of SEQ ID NO:983. In one aspect, provided herein is an antibody that binds TRGV9, comprising a VL having an amino acid sequence having at least 95% identity to an amino acid sequence of SEQ ID NO:984. In one aspect, provided herein is an antibody that binds TRGV9, comprising a VH having an amino acid sequence having at least 95% identity to an amino acid sequence of SEQ ID NO:983, and a VL having an amino acid sequence having at least 95% identity to an amino acid sequence of SEQ ID NO:984. In one aspect, provided herein is an antibody that binds TRGV9, comprising a heavy chain having an amino acid sequence having at least 95% identity to an amino acid sequence of SEQ ID NO:985. In one aspect, provided herein is an antibody that binds TRGV9, comprising a light chain having an amino acid sequence having at least 95% identity to an amino acid sequence of SEQ ID NO:986. In one aspect, provided herein is an antibody that binds TRGV9, comprising a heavy chain having an amino acid sequence having at least 95% identity to an amino acid sequence of SEQ ID NO:985, and a light chain having an amino acid sequence having at least 95% identity to an amino acid sequence of SEQ ID NO:986.

In one aspect, provided herein is an antibody that binds TRGV9, comprising: (i) a VH comprising a VH CDR1, a VH CDR2, and a VH CDR3 having an amino acid sequence of SEQ ID NOs:987, 988, and 989, respectively, and (ii) a VL comprising a VL CDR1, VL CDR2, and VL CDR3 having an amino acid sequence of SEQ ID NOs:990, 991, and 992, respectively. In one aspect, provided herein is an antibody that binds TRGV9, comprising: (i) a VH comprising a VH CDR1, a VH CDR2, and a VH CDR3 having an amino acid sequence of SEQ ID NOs:993, 994, and 995, respectively, and (ii) a VL comprising a VL CDR1, VL CDR2, and VL CDR3 having an amino acid sequence of SEQ ID NOs:996, 997, and 998, respectively. In one aspect, provided herein is an antibody that binds TRGV9, comprising: (i) a VH comprising a VH CDR1, a VH CDR2, and a VH CDR3 having an amino acid sequence of SEQ ID NOs:999, 1000, and 1001, respectively, and (ii) a VL comprising a VL CDR1, VL CDR2, and VL CDR3 having an amino acid sequence of SEQ ID NOs:1002, 1003, and 1004, respectively. In one aspect, provided herein is an antibody that binds TRGV9, comprising: (i) a VH comprising a VH CDR1, a VH CDR2, and a VH CDR3 having an amino acid sequence of SEQ ID NOs:1005, 1006, and 1007, respectively, and (ii) a VL comprising a VL CDR1, VL CDR2, and VL CDR3 having an amino acid sequence of SEQ ID NOs:1008, 1009, and 1010, respectively. In one aspect, provided herein is an antibody that binds TRGV9, comprising: (i) a VH comprising a VH CDR1, a VH CDR2, and a VH CDR3 having an amino acid sequence of SEQ ID NOs:1011, 1012, and 1013, respectively, and (ii) a VL comprising a VL CDR1, VL CDR2, and VL CDR3 having an amino acid sequence of SEQ ID NOs:1014, 1015, and 1016, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1017; and (ii) a VL comprising a VL CDR1, a VL CDR2, and a VL CDR3 having an amino acid sequence of a VL CDR1, a VL CDR2, and a VL CDR3, respectively, of SEQ ID NO:1018. In one aspect, provided herein is an antibody that binds TRGV9, comprising a VH having an amino acid sequence of SEQ ID NO:1017. In one aspect, provided herein is an antibody that binds TRGV9, comprising a VL having an amino acid sequence of SEQ ID NO:1018. In one aspect, provided herein is an antibody that binds TRGV9, comprising a VH having an amino acid sequence of SEQ ID NO:1017, and a VL having an amino acid sequence of SEQ ID NO:1018. In one aspect, provided herein is an antibody that binds TRGV9, comprising a heavy chain having an amino acid sequence of SEQ ID NO:1019. In one aspect, provided herein is an antibody that binds TRGV9, comprising a light chain having an amino acid sequence of SEQ ID NO:1020. In one aspect, provided herein is an antibody that binds TRGV9, comprising a heavy chain having an amino acid sequence of SEQ ID NO:1019, and a light chain having an amino acid sequence of SEQ ID NO:1020. In one aspect, provided herein is an antibody that binds TRGV9, comprising a VH having an amino acid sequence having at least 95% identity to an amino acid sequence of SEQ ID NO:1017. In one aspect, provided herein is an antibody that binds TRGV9, comprising a VL having an amino acid sequence having at least 95% identity to an amino acid sequence of SEQ ID NO:1018. In one aspect, provided herein is an antibody that binds TRGV9, comprising a VH having an amino acid sequence having at least 95% identity to an amino acid sequence of SEQ ID NO:1017, and a VL having an amino acid sequence having at least 95% identity to an amino acid sequence of SEQ ID NO:1018. In one aspect, provided herein is an antibody that binds TRGV9, comprising a heavy chain having an amino acid sequence having at least 95% identity to an amino acid sequence of SEQ ID NO:1019. In one aspect, provided herein is an antibody that binds TRGV9, comprising a light chain having an amino acid sequence having at least 95% identity to an amino acid sequence of SEQ ID NO:1020. In one aspect, provided herein is an antibody that binds TRGV9, comprising a heavy chain having an amino acid sequence having at least 95% identity to an amino acid sequence of SEQ ID NO:1019, and a light chain having an amino acid sequence having at least 95% identity to an amino acid sequence of SEQ ID NO:1020.

In one aspect, provided herein is an antibody that binds TRGV9, comprising: (i) a VH comprising a VH CDR1, a VH CDR2, and a VH CDR3 having an amino acid sequence of SEQ ID NOs:1021, 1022, and 1023, respectively, and (ii) a VL comprising a VL CDR1, VL CDR2, and VL CDR3 having an amino acid sequence of SEQ ID NOs:1024, 1025, and 1026, respectively. In one aspect, provided herein is an antibody that binds TRGV9, comprising: (i) a VH comprising a VH CDR1, a VH CDR2, and a VH CDR3 having an amino acid sequence of SEQ ID NOs:1027, 1028, and 1029, respectively, and (ii) a VL comprising a VL CDR1, VL CDR2, and VL CDR3 having an amino acid sequence of SEQ ID NOs:1030, 1031, and 1032, respectively. In one aspect, provided herein is an antibody that binds TRGV9, comprising: (i) a VH comprising a VH CDR1, a VH CDR2, and a VH CDR3 having an amino acid sequence of SEQ ID NOs:1033, 1034, and 1035, respectively, and (ii) a VL comprising a VL CDR1, VL CDR2, and VL CDR3 having an amino acid sequence of SEQ ID NOs:1036, 1037, and 1038, respectively. In one aspect, provided herein is an antibody that binds TRGV9, comprising: (i) a VH comprising a VH CDR1, a VH CDR2, and a VH CDR3 having an amino acid sequence of SEQ ID NOs:1039, 1040, and 1041, respectively, and (ii) a VL comprising a VL CDR1, VL CDR2, and VL CDR3 having an amino acid sequence of SEQ ID NOs:1042, 1043, and 1044, respectively. In one aspect, provided herein is an antibody that binds TRGV9, comprising: (i) a VH comprising a VH CDR1, a VH CDR2, and a VH CDR3 having an amino acid sequence of SEQ ID NOs:1045, 1046, and 1047, respectively, and (ii) a VL comprising a VL CDR1, VL CDR2, and VL CDR3 having an amino acid sequence of SEQ ID NOs:1048, 1049, and 1050, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1051; and (ii) a VL comprising a VL CDR1, a VL CDR2, and a VL CDR3 having an amino acid sequence of a VL CDR1, a VL CDR2, and a VL CDR3, respectively, of SEQ ID NO:1052. In one aspect, provided herein is an antibody that binds TRGV9, comprising a VH having an amino acid sequence of SEQ ID NO:1051. In one aspect, provided herein is an antibody that binds TRGV9, comprising a VL having an amino acid sequence of SEQ ID NO:1052. In one aspect, provided herein is an antibody that binds TRGV9, comprising a VH having an amino acid sequence of SEQ ID NO:1051, and a VL having an amino acid sequence of SEQ ID NO:1052. In one aspect, provided herein is an antibody that binds TRGV9, comprising a heavy chain having an amino acid sequence of SEQ ID NO:1053. In one aspect, provided herein is an antibody that binds TRGV9, comprising a light chain having an amino acid sequence of SEQ ID NO:1054. In one aspect, provided herein is an antibody that binds TRGV9, comprising a heavy chain having an amino acid sequence of SEQ ID NO:1053, and a light chain having an amino acid sequence of SEQ ID NO:1054. In one aspect, provided herein is an antibody that binds TRGV9, comprising a VH having an amino acid sequence having at least 95% identity to an amino acid sequence of SEQ ID NO:1051. In one aspect, provided herein is an antibody that binds TRGV9, comprising a VL having an amino acid sequence having at least 95% identity to an amino acid sequence of SEQ ID NO:1052. In one aspect, provided herein is an antibody that binds TRGV9, comprising a VH having an amino acid sequence having at least 95% identity to an amino acid sequence of SEQ ID NO:1051, and a VL having an amino acid sequence having at least 95% identity to an amino acid sequence of SEQ ID NO:1052. In one aspect, provided herein is an antibody that binds TRGV9, comprising a heavy chain having an amino acid sequence having at least 95% identity to an amino acid sequence of SEQ ID NO:1053. In one aspect, provided herein is an antibody that binds TRGV9, comprising a light chain having an amino acid sequence having at least 95% identity to an amino acid sequence of SEQ ID NO:1054. In one aspect, provided herein is an antibody that binds TRGV9, comprising a heavy chain having an amino acid sequence having at least 95% identity to an amino acid sequence of SEQ ID NO:1053, and a light chain having an amino acid sequence having at least 95% identity to an amino acid sequence of SEQ ID NO:1054.

In one aspect, provided herein is an antibody that binds TRGV9, comprising: (i) a VH comprising a VH CDR1, a VH CDR2, and a VH CDR3 having an amino acid sequence of SEQ ID NOs:1055, 1056, and 1057, respectively, and (ii) a VL comprising a VL CDR1, VL CDR2, and VL CDR3 having an amino acid sequence of SEQ ID NOs:1058, 1059, and 1060, respectively. In one aspect, provided herein is an antibody that binds TRGV9, comprising: (i) a VH comprising a VH CDR1, a VH CDR2, and a VH CDR3 having an amino acid sequence of SEQ ID NOs:1061, 1062, and 1063, respectively, and (ii) a VL comprising a VL CDR1, VL CDR2, and VL CDR3 having an amino acid sequence of SEQ ID NOs:1064, 1065, and 1066, respectively. In one aspect, provided herein is an antibody that binds TRGV9, comprising: (i) a VH comprising a VH CDR1, a VH CDR2, and a VH CDR3 having an amino acid sequence of SEQ ID NOs:1067, 1068, and 1069, respectively, and (ii) a VL comprising a VL CDR1, VL CDR2, and VL CDR3 having an amino acid sequence of SEQ ID NOs:1070, 1071, and 1072, respectively. In one aspect, provided herein is an antibody that binds TRGV9, comprising: (i) a VH comprising a VH CDR1, a VH CDR2, and a VH CDR3 having an amino acid sequence of SEQ ID NOs:1073, 1074, and 1075, respectively, and (ii) a VL comprising a VL CDR1, VL CDR2, and VL CDR3 having an amino acid sequence of SEQ ID NOs:1076, 1077, and 1078, respectively. In one aspect, provided herein is an antibody that binds TRGV9, comprising: (i) a VH comprising a VH CDR1, a VH CDR2, and a VH CDR3 having an amino acid sequence of SEQ ID NOs:1079, 1080, and 1081, respectively, and (ii) a VL comprising a VL CDR1, VL CDR2, and VL CDR3 having an amino acid sequence of SEQ ID NOs:1082, 1083, and 1084, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1085; and (ii) a VL comprising a VL CDR1, a VL CDR2, and a VL CDR3 having an amino acid sequence of a VL CDR1, a VL CDR2, and a VL CDR3, respectively, of SEQ ID NO:1086. In one aspect, provided herein is an antibody that binds TRGV9, comprising a VH having an amino acid sequence of SEQ ID NO:1085. In one aspect, provided herein is an antibody that binds TRGV9, comprising a VL having an amino acid sequence of SEQ ID NO:1086. In one aspect, provided herein is an antibody that binds TRGV9, comprising a VH having an amino acid sequence of SEQ ID NO:1085, and a VL having an amino acid sequence of SEQ ID NO:1086. In one aspect, provided herein is an antibody that binds TRGV9, comprising a heavy chain having an amino acid sequence of SEQ ID NO:1087. In one aspect, provided herein is an antibody that binds TRGV9, comprising a light chain having an amino acid sequence of SEQ ID NO:1088. In one aspect, provided herein is an antibody that binds TRGV9, comprising a heavy chain having an amino acid sequence of SEQ ID NO:1087, and a light chain having an amino acid sequence of SEQ ID NO:1088. In one aspect, provided herein is an antibody that binds TRGV9, comprising a VH having an amino acid sequence having at least 95% identity to an amino acid sequence of SEQ ID NO:1085. In one aspect, provided herein is an antibody that binds TRGV9, comprising a VL having an amino acid sequence having at least 95% identity to an amino acid sequence of SEQ ID NO:1086. In one aspect, provided herein is an antibody that binds TRGV9, comprising a VH having an amino acid sequence having at least 95% identity to an amino acid sequence of SEQ ID NO:1085, and a VL having an amino acid sequence having at least 95% identity to an amino acid sequence of SEQ ID NO:1086. In one aspect, provided herein is an antibody that binds TRGV9, comprising a heavy chain having an amino acid sequence having at least 95% identity to an amino acid sequence of SEQ ID NO:1087. In one aspect, provided herein is an antibody that binds TRGV9, comprising a light chain having an amino acid sequence having at least 95% identity to an amino acid sequence of SEQ ID NO:1088. In one aspect, provided herein is an antibody that binds TRGV9, comprising a heavy chain having an amino acid sequence having at least 95% identity to an amino acid sequence of SEQ ID NO:1087, and a light chain having an amino acid sequence having at least 95% identity to an amino acid sequence of SEQ ID NO:1088.

In one aspect, provided herein is an antibody that binds TRGV9, comprising: (i) a VH comprising a VH CDR1, a VH CDR2, and a VH CDR3 having an amino acid sequence of SEQ ID NOs:1089, 1090, and 1091, respectively, and (ii) a VL comprising a VL CDR1, VL CDR2, and VL CDR3 having an amino acid sequence of SEQ ID NOs:1092, 1093, and 1094, respectively. In one aspect, provided herein is an antibody that binds TRGV9, comprising: (i) a VH comprising a VH CDR1, a VH CDR2, and a VH CDR3 having an amino acid sequence of SEQ ID NOs:1095, 1096, and 1097, respectively, and (ii) a VL comprising a VL CDR1, VL CDR2, and VL CDR3 having an amino acid sequence of SEQ ID NOs:1098, 1099, and 1100, respectively. In one aspect, provided herein is an antibody that binds TRGV9, comprising: (i) a VH comprising a VH CDR1, a VH CDR2, and a VH CDR3 having an amino acid sequence of SEQ ID NOs:1101, 1102, and 1103, respectively, and (ii) a VL comprising a VL CDR1, VL CDR2, and VL CDR3 having an amino acid sequence of SEQ ID NOs:1104, 1105, and 1106, respectively. In one aspect, provided herein is an antibody that binds TRGV9, comprising: (i) a VH comprising a VH CDR1, a VH CDR2, and a VH CDR3 having an amino acid sequence of SEQ ID NOs:1107, 1108, and 1109, respectively, and (ii) a VL comprising a VL CDR1, VL CDR2, and VL CDR3 having an amino acid sequence of SEQ ID NOs:1110, 1111, and 1112, respectively. In one aspect, provided herein is an antibody that binds TRGV9, comprising: (i) a VH comprising a VH CDR1, a VH CDR2, and a VH CDR3 having an amino acid sequence of SEQ ID NOs:1113, 1114, and 1115, respectively, and (ii) a VL comprising a VL CDR1, VL CDR2, and VL CDR3 having an amino acid sequence of SEQ ID NOs:1116, 1117, and 1118, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1119; and (ii) a VL comprising a VL CDR1, a VL CDR2, and a VL CDR3 having an amino acid sequence of a VL CDR1, a VL CDR2, and a VL CDR3, respectively, of SEQ ID NO:1120. In one aspect, provided herein is an antibody that binds TRGV9, comprising a VH having an amino acid sequence of SEQ ID NO:1119. In one aspect, provided herein is an antibody that binds TRGV9, comprising a VL having an amino acid sequence of SEQ ID NO:1120. In one aspect, provided herein is an antibody that binds TRGV9, comprising a VH having an amino acid sequence of SEQ ID NO:1119, and a VL having an amino acid sequence of SEQ ID NO:1120. In one aspect, provided herein is an antibody that binds TRGV9, comprising a heavy chain having an amino acid sequence of SEQ ID NO:1121. In one aspect, provided herein is an antibody that binds TRGV9, comprising a light chain having an amino acid sequence of SEQ ID NO:1122. In one aspect, provided herein is an antibody that binds TRGV9, comprising a heavy chain having an amino acid sequence of SEQ ID NO:1121, and a light chain having an amino acid sequence of SEQ ID NO:1122. In one aspect, provided herein is an antibody that binds TRGV9, comprising a VH having an amino acid sequence having at least 95% identity to an amino acid sequence of SEQ ID NO:1119. In one aspect, provided herein is an antibody that binds TRGV9, comprising a VL having an amino acid sequence having at least 95% identity to an amino acid sequence of SEQ ID NO:1120. In one aspect, provided herein is an antibody that binds TRGV9, comprising a VH having an amino acid sequence having at least 95% identity to an amino acid sequence of SEQ ID NO:1119, and a VL having an amino acid sequence having at least 95% identity to an amino acid sequence of SEQ ID NO:1120. In one aspect, provided herein is an antibody that binds TRGV9, comprising a heavy chain having an amino acid sequence having at least 95% identity to an amino acid sequence of SEQ ID NO:1121. In one aspect, provided herein is an antibody that binds TRGV9, comprising a light chain having an amino acid sequence having at least 95% identity to an amino acid sequence of SEQ ID NO:1122. In one aspect, provided herein is an antibody that binds TRGV9, comprising a heavy chain having an amino acid sequence having at least 95% identity to an amino acid sequence of SEQ ID NO:1121, and a light chain having an amino acid sequence having at least 95% identity to an amino acid sequence of SEQ ID NO:1122.

In one aspect, provided herein is an antibody that binds TRGV9, comprising: (i) a VH comprising a VH CDR1, a VH CDR2, and a VH CDR3 having an amino acid sequence of SEQ ID NOs:1123, 1124, and 1125, respectively, and (ii) a VL comprising a VL CDR1, VL CDR2, and VL CDR3 having an amino acid sequence of SEQ ID NOs:1126, 1127, and 1128, respectively. In one aspect, provided herein is an antibody that binds TRGV9, comprising: (i) a VH comprising a VH CDR1, a VH CDR2, and a VH CDR3 having an amino acid sequence of SEQ ID NOs:1129, 1130, and 1131, respectively, and (ii) a VL comprising a VL CDR1, VL CDR2, and VL CDR3 having an amino acid sequence of SEQ ID NOs:1132, 1133, and 1134, respectively. In one aspect, provided herein is an antibody that binds TRGV9, comprising: (i) a VH comprising a VH CDR1, a VH CDR2, and a VH CDR3 having an amino acid sequence of SEQ ID NOs:1135, 1136, and 1137, respectively, and (ii) a VL comprising a VL CDR1, VL CDR2, and VL CDR3 having an amino acid sequence of SEQ ID NOs:1138, 1139, and 1140, respectively. In one aspect, provided herein is an antibody that binds TRGV9, comprising: (i) a VH comprising a VH CDR1, a VH CDR2, and a VH CDR3 having an amino acid sequence of SEQ ID NOs:1141, 1142, and 1143, respectively, and (ii) a VL comprising a VL CDR1, VL CDR2, and VL CDR3 having an amino acid sequence of SEQ ID NOs:1144, 1145, and 1146, respectively. In one aspect, provided herein is an antibody that binds TRGV9, comprising: (i) a VH comprising a VH CDR1, a VH CDR2, and a VH CDR3 having an amino acid sequence of SEQ ID NOs:1147, 1148, and 1149, respectively, and (ii) a VL comprising a VL CDR1, VL CDR2, and VL CDR3 having an amino acid sequence of SEQ ID NOs:1150, 1151, and 1152, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1153; and (ii) a VL comprising a VL CDR1, a VL CDR2, and a VL CDR3 having an amino acid sequence of a VL CDR1, a VL CDR2, and a VL CDR3, respectively, of SEQ ID NO:1154. In one aspect, provided herein is an antibody that binds TRGV9, comprising a VH having an amino acid sequence of SEQ ID NO:1153. In one aspect, provided herein is an antibody that binds TRGV9, comprising a VL having an amino acid sequence of SEQ ID NO:1154. In one aspect, provided herein is an antibody that binds TRGV9, comprising a VH having an amino acid sequence of SEQ ID NO:1153, and a VL having an amino acid sequence of SEQ ID NO:1154. In one aspect, provided herein is an antibody that binds TRGV9, comprising a heavy chain having an amino acid sequence of SEQ ID NO:1155. In one aspect, provided herein is an antibody that binds TRGV9, comprising a light chain having an amino acid sequence of SEQ ID NO:1156. In one aspect, provided herein is an antibody that binds TRGV9, comprising a heavy chain having an amino acid sequence of SEQ ID NO:1155, and a light chain having an amino acid sequence of SEQ ID NO:1156. In one aspect, provided herein is an antibody that binds TRGV9, comprising a VH having an amino acid sequence having at least 95% identity to an amino acid sequence of SEQ ID NO:1153. In one aspect, provided herein is an antibody that binds TRGV9, comprising a VL having an amino acid sequence having at least 95% identity to an amino acid sequence of SEQ ID NO:1154. In one aspect, provided herein is an antibody that binds TRGV9, comprising a VH having an amino acid sequence having at least 95% identity to an amino acid sequence of SEQ ID NO:1153, and a VL having an amino acid sequence having at least 95% identity to an amino acid sequence of SEQ ID NO:1154. In one aspect, provided herein is an antibody that binds TRGV9, comprising a heavy chain having an amino acid sequence having at least 95% identity to an amino acid sequence of SEQ ID NO:1155. In one aspect, provided herein is an antibody that binds TRGV9, comprising a light chain having an amino acid sequence having at least 95% identity to an amino acid sequence of SEQ ID NO:1156. In one aspect, provided herein is an antibody that binds TRGV9, comprising a heavy chain having an amino acid sequence having at least 95% identity to an amino acid sequence of SEQ ID NO:1155, and a light chain having an amino acid sequence having at least 95% identity to an amino acid sequence of SEQ ID NO:1156.

In one aspect, provided herein is an antibody that binds TRGV9, comprising: (i) a VH comprising a VH CDR1, a VH CDR2, and a VH CDR3 having an amino acid sequence of SEQ ID NOs:1157, 1158, and 1159, respectively, and (ii) a VL comprising a VL CDR1, VL CDR2, and VL CDR3 having an amino acid sequence of SEQ ID NOs:1160, 1161, and 1162, respectively. In one aspect, provided herein is an antibody that binds TRGV9, comprising: (i) a VH comprising a VH CDR1, a VH CDR2, and a VH CDR3 having an amino acid sequence of SEQ ID NOs:1163, 1164, and 1165, respectively, and (ii) a VL comprising a VL CDR1, VL CDR2, and VL CDR3 having an amino acid sequence of SEQ ID NOs:1166, 1167, and 1168, respectively. In one aspect, provided herein is an antibody that binds TRGV9, comprising: (i) a VH comprising a VH CDR1, a VH CDR2, and a VH CDR3 having an amino acid sequence of SEQ ID NOs:1169, 1170, and 1171, respectively, and (ii) a VL comprising a VL CDR1, VL CDR2, and VL CDR3 having an amino acid sequence of SEQ ID NOs:1172, 1173, and 1174, respectively. In one aspect, provided herein is an antibody that binds TRGV9, comprising: (i) a VH comprising a VH CDR1, a VH CDR2, and a VH CDR3 having an amino acid sequence of SEQ ID NOs:1175, 1176, and 1177, respectively, and (ii) a VL comprising a VL CDR1, VL CDR2, and VL CDR3 having an amino acid sequence of SEQ ID NOs:1178, 1179, and 1180, respectively. In one aspect, provided herein is an antibody that binds TRGV9, comprising: (i) a VH comprising a VH CDR1, a VH CDR2, and a VH CDR3 having an amino acid sequence of SEQ ID NOs:1181, 1182, and 1183, respectively, and (ii) a VL comprising a VL CDR1, VL CDR2, and VL CDR3 having an amino acid sequence of SEQ ID NOs:1184, 1185, and 1186, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1187; and (ii) a VL comprising a VL CDR1, a VL CDR2, and a VL CDR3 having an amino acid sequence of a VL CDR1, a VL CDR2, and a VL CDR3, respectively, of SEQ ID NO:1188. In one aspect, provided herein is an antibody that binds TRGV9, comprising a VH having an amino acid sequence of SEQ ID NO:1187. In one aspect, provided herein is an antibody that binds TRGV9, comprising a VL having an amino acid sequence of SEQ ID NO:1188. In one aspect, provided herein is an antibody that binds TRGV9, comprising a VH having an amino acid sequence of SEQ ID NO:1187, and a VL having an amino acid sequence of SEQ ID NO:1188. In one aspect, provided herein is an antibody that binds TRGV9, comprising a heavy chain having an amino acid sequence of SEQ ID NO:1189. In one aspect, provided herein is an antibody that binds TRGV9, comprising a light chain having an amino acid sequence of SEQ ID NO:1190. In one aspect, provided herein is an antibody that binds TRGV9, comprising a heavy chain having an amino acid sequence of SEQ ID NO:1189, and a light chain having an amino acid sequence of SEQ ID NO:1190. In one aspect, provided herein is an antibody that binds TRGV9, comprising a VH having an amino acid sequence having at least 95% identity to an amino acid sequence of SEQ ID NO:1187. In one aspect, provided herein is an antibody that binds TRGV9, comprising a VL having an amino acid sequence having at least 95% identity to an amino acid sequence of SEQ ID NO:1188. In one aspect, provided herein is an antibody that binds TRGV9, comprising a VH having an amino acid sequence having at least 95% identity to an amino acid sequence of SEQ ID NO:1187, and a VL having an amino acid sequence having at least 95% identity to an amino acid sequence of SEQ ID NO:1188. In one aspect, provided herein is an antibody that binds TRGV9, comprising a heavy chain having an amino acid sequence having at least 95% identity to an amino acid sequence of SEQ ID NO:1189. In one aspect, provided herein is an antibody that binds TRGV9, comprising a light chain having an amino acid sequence having at least 95% identity to an amino acid sequence of SEQ ID NO:1190. In one aspect, provided herein is an antibody that binds TRGV9, comprising a heavy chain having an amino acid sequence having at least 95% identity to an amino acid sequence of SEQ ID NO:1189, and a light chain having an amino acid sequence having at least 95% identity to an amino acid sequence of SEQ ID NO:1190.

In one aspect, provided herein is an antibody that binds TRGV9, comprising: (i) a VH comprising a VH CDR1, a VH CDR2, and a VH CDR3 having an amino acid sequence of SEQ ID NOs:1191, 1192, and 1193, respectively, and (ii) a VL comprising a VL CDR1, VL CDR2, and VL CDR3 having an amino acid sequence of SEQ ID NOs:1194, 1195, and 1196, respectively. In one aspect, provided herein is an antibody that binds TRGV9, comprising: (i) a VH comprising a VH CDR1, a VH CDR2, and a VH CDR3 having an amino acid sequence of SEQ ID NOs:1197, 1198, and 1199, respectively, and (ii) a VL comprising a VL CDR1, VL CDR2, and VL CDR3 having an amino acid sequence of SEQ ID NOs:1200, 1201, and 1202, respectively. In one aspect, provided herein is an antibody that binds TRGV9, comprising: (i) a VH comprising a VH CDR1, a VH CDR2, and a VH CDR3 having an amino acid sequence of SEQ ID NOs:1203, 1204, and 1205, respectively, and (ii) a VL comprising a VL CDR1, VL CDR2, and VL CDR3 having an amino acid sequence of SEQ ID NOs:1206, 1207, and 1208, respectively. In one aspect, provided herein is an antibody that binds TRGV9, comprising: (i) a VH comprising a VH CDR1, a VH CDR2, and a VH CDR3 having an amino acid sequence of SEQ ID NOs:1209, 1210, and 1211, respectively, and (ii) a VL comprising a VL CDR1, VL CDR2, and VL CDR3 having an amino acid sequence of SEQ ID NOs:1212, 1213, and 1214, respectively. In one aspect, provided herein is an antibody that binds TRGV9, comprising: (i) a VH comprising a VH CDR1, a VH CDR2, and a VH CDR3 having an amino acid sequence of SEQ ID NOs:1215, 1216, and 1217, respectively, and (ii) a VL comprising a VL CDR1, VL CDR2, and VL CDR3 having an amino acid sequence of SEQ ID NOs:1218, 1219, and 1220, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1221; and (ii) a VL comprising a VL CDR1, a VL CDR2, and a VL CDR3 having an amino acid sequence of a VL CDR1, a VL CDR2, and a VL CDR3, respectively, of SEQ ID NO:1222. In one aspect, provided herein is an antibody that binds TRGV9, comprising a VH having an amino acid sequence of SEQ ID NO:1221. In one aspect, provided herein is an antibody that binds TRGV9, comprising a VL having an amino acid sequence of SEQ ID NO:1222. In one aspect, provided herein is an antibody that binds TRGV9, comprising a VH having an amino acid sequence of SEQ ID NO:1221, and a VL having an amino acid sequence of SEQ ID NO:1222. In one aspect, provided herein is an antibody that binds TRGV9, comprising a heavy chain having an amino acid sequence of SEQ ID NO:1223. In one aspect, provided herein is an antibody that binds TRGV9, comprising a light chain having an amino acid sequence of SEQ ID NO:1224. In one aspect, provided herein is an antibody that binds TRGV9, comprising a heavy chain having an amino acid sequence of SEQ ID NO:1223, and a light chain having an amino acid sequence of SEQ ID NO:1224. In one aspect, provided herein is an antibody that binds TRGV9, comprising a VH having an amino acid sequence having at least 95% identity to an amino acid sequence of SEQ ID NO:1221. In one aspect, provided herein is an antibody that binds TRGV9, comprising a VL having an amino acid sequence having at least 95% identity to an amino acid sequence of SEQ ID NO:1222. In one aspect, provided herein is an antibody that binds TRGV9, comprising a VH having an amino acid sequence having at least 95% identity to an amino acid sequence of SEQ ID NO:1221, and a VL having an amino acid sequence having at least 95% identity to an amino acid sequence of SEQ ID NO:1222. In one aspect, provided herein is an antibody that binds TRGV9, comprising a heavy chain having an amino acid sequence having at least 95% identity to an amino acid sequence of SEQ ID NO:1223. In one aspect, provided herein is an antibody that binds TRGV9, comprising a light chain having an amino acid sequence having at least 95% identity to an amino acid sequence of SEQ ID NO:1224. In one aspect, provided herein is an antibody that binds TRGV9, comprising a heavy chain having an amino acid sequence having at least 95% identity to an amino acid sequence of SEQ ID NO:1223, and a light chain having an amino acid sequence having at least 95% identity to an amino acid sequence of SEQ ID NO:1224.

In one aspect, provided herein is an antibody that binds TRGV9, comprising: (i) a VH comprising a VH CDR1, a VH CDR2, and a VH CDR3 having an amino acid sequence of SEQ ID NOs:1225, 1226, and 1227, respectively, and (ii) a VL comprising a VL CDR1, VL CDR2, and VL CDR3 having an amino acid sequence of SEQ ID NOs:1228, 1229, and 1230, respectively. In one aspect, provided herein is an antibody that binds TRGV9, comprising: (i) a VH comprising a VH CDR1, a VH CDR2, and a VH CDR3 having an amino acid sequence of SEQ ID NOs:1231, 1232, and 1233, respectively, and (ii) a VL comprising a VL CDR1, VL CDR2, and VL CDR3 having an amino acid sequence of SEQ ID NOs:1234, 1235, and 1236, respectively. In one aspect, provided herein is an antibody that binds TRGV9, comprising: (i) a VH comprising a VH CDR1, a VH CDR2, and a VH CDR3 having an amino acid sequence of SEQ ID NOs:1237, 1238, and 1239, respectively, and (ii) a VL comprising a VL CDR1, VL CDR2, and VL CDR3 having an amino acid sequence of SEQ ID NOs:1240, 1241, and 1242, respectively. In one aspect, provided herein is an antibody that binds TRGV9, comprising: (i) a VH comprising a VH CDR1, a VH CDR2, and a VH CDR3 having an amino acid sequence of SEQ ID NOs:1243, 1244, and 1245, respectively, and (ii) a VL comprising a VL CDR1, VL CDR2, and VL CDR3 having an amino acid sequence of SEQ ID NOs:1246, 1247, and 1248, respectively. In one aspect, provided herein is an antibody that binds TRGV9, comprising: (i) a VH comprising a VH CDR1, a VH CDR2, and a VH CDR3 having an amino acid sequence of SEQ ID NOs:1249, 1250, and 1251, respectively, and (ii) a VL comprising a VL CDR1, VL CDR2, and VL CDR3 having an amino acid sequence of SEQ ID NOs:1252, 1253, and 1254, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1255; and (ii) a VL comprising a VL CDR1, a VL CDR2, and a VL CDR3 having an amino acid sequence of a VL CDR1, a VL CDR2, and a VL CDR3, respectively, of SEQ ID NO:1256. In one aspect, provided herein is an antibody that binds TRGV9, comprising a VH having an amino acid sequence of SEQ ID NO:1255. In one aspect, provided herein is an antibody that binds TRGV9, comprising a VL having an amino acid sequence of SEQ ID NO:1256. In one aspect, provided herein is an antibody that binds TRGV9, comprising a VH having an amino acid sequence of SEQ ID NO:1255, and a VL having an amino acid sequence of SEQ ID NO:1256. In one aspect, provided herein is an antibody that binds TRGV9, comprising a heavy chain having an amino acid sequence of SEQ ID NO:1257. In one aspect, provided herein is an antibody that binds TRGV9, comprising a light chain having an amino acid sequence of SEQ ID NO:1258. In one aspect, provided herein is an antibody that binds TRGV9, comprising a heavy chain having an amino acid sequence of SEQ ID NO:1257, and a light chain having an amino acid sequence of SEQ ID NO:1258. In one aspect, provided herein is an antibody that binds TRGV9, comprising a VH having an amino acid sequence having at least 95% identity to an amino acid sequence of SEQ ID NO:1255. In one aspect, provided herein is an antibody that binds TRGV9, comprising a VL having an amino acid sequence having at least 95% identity to an amino acid sequence of SEQ ID NO:1256. In one aspect, provided herein is an antibody that binds TRGV9, comprising a VH having an amino acid sequence having at least 95% identity to an amino acid sequence of SEQ ID NO:1255, and a VL having an amino acid sequence having at least 95% identity to an amino acid sequence of SEQ ID NO:1256. In one aspect, provided herein is an antibody that binds TRGV9, comprising a heavy chain having an amino acid sequence having at least 95% identity to an amino acid sequence of SEQ ID NO:1257. In one aspect, provided herein is an antibody that binds TRGV9, comprising a light chain having an amino acid sequence having at least 95% identity to an amino acid sequence of SEQ ID NO:1258. In one aspect, provided herein is an antibody that binds TRGV9, comprising a heavy chain having an amino acid sequence having at least 95% identity to an amino acid sequence of SEQ ID NO:1257, and a light chain having an amino acid sequence having at least 95% identity to an amino acid sequence of SEQ ID NO:1258.

In one aspect, provided herein is an antibody that binds TRGV9, comprising: (i) a VH comprising a VH CDR1, a VH CDR2, and a VH CDR3 having an amino acid sequence of SEQ ID NOs:1259, 1260, and 1261, respectively, and (ii) a VL comprising a VL CDR1, VL CDR2, and VL CDR3 having an amino acid sequence of SEQ ID NOs:1262, 1263, and 1264, respectively. In one aspect, provided herein is an antibody that binds TRGV9, comprising: (i) a VH comprising a VH CDR1, a VH CDR2, and a VH CDR3 having an amino acid sequence of SEQ ID NOs:1265, 1266, and 1267, respectively, and (ii) a VL comprising a VL CDR1, VL CDR2, and VL CDR3 having an amino acid sequence of SEQ ID NOs:1268, 1269, and 1270, respectively. In one aspect, provided herein is an antibody that binds TRGV9, comprising: (i) a VH comprising a VH CDR1, a VH CDR2, and a VH CDR3 having an amino acid sequence of SEQ ID NOs:1271, 1272, and 1273, respectively, and (ii) a VL comprising a VL CDR1, VL CDR2, and VL CDR3 having an amino acid sequence of SEQ ID NOs:1274, 1275, and 1276, respectively. In one aspect, provided herein is an antibody that binds TRGV9, comprising: (i) a VH comprising a VH CDR1, a VH CDR2, and a VH CDR3 having an amino acid sequence of SEQ ID NOs:1277, 1278, and 1279, respectively, and (ii) a VL comprising a VL CDR1, VL CDR2, and VL CDR3 having an amino acid sequence of SEQ ID NOs:1280, 1281, and 1282, respectively. In one aspect, provided herein is an antibody that binds TRGV9, comprising: (i) a VH comprising a VH CDR1, a VH CDR2, and a VH CDR3 having an amino acid sequence of SEQ ID NOs:1283, 1284, and 1285, respectively, and (ii) a VL comprising a VL CDR1, VL CDR2, and VL CDR3 having an amino acid sequence of SEQ ID NOs:1286, 1287, and 1288, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1289; and (ii) a VL comprising a VL CDR1, a VL CDR2, and a VL CDR3 having an amino acid sequence of a VL CDR1, a VL CDR2, and a VL CDR3, respectively, of SEQ ID NO:1290. In one aspect, provided herein is an antibody that binds TRGV9, comprising a VH having an amino acid sequence of SEQ ID NO:1289. In one aspect, provided herein is an antibody that binds TRGV9, comprising a VL having an amino acid sequence of SEQ ID NO:1290. In one aspect, provided herein is an antibody that binds TRGV9, comprising a VH having an amino acid sequence of SEQ ID NO:1289, and a VL having an amino acid sequence of SEQ ID NO:1290. In one aspect, provided herein is an antibody that binds TRGV9, comprising a heavy chain having an amino acid sequence of SEQ ID NO:1291. In one aspect, provided herein is an antibody that binds TRGV9, comprising a light chain having an amino acid sequence of SEQ ID NO:1292. In one aspect, provided herein is an antibody that binds TRGV9, comprising a heavy chain having an amino acid sequence of SEQ ID NO:1291, and a light chain having an amino acid sequence of SEQ ID NO:1292. In one aspect, provided herein is an antibody that binds TRGV9, comprising a VH having an amino acid sequence having at least 95% identity to an amino acid sequence of SEQ ID NO:1289. In one aspect, provided herein is an antibody that binds TRGV9, comprising a VL having an amino acid sequence having at least 95% identity to an amino acid sequence of SEQ ID NO:1290. In one aspect, provided herein is an antibody that binds TRGV9, comprising a VH having an amino acid sequence having at least 95% identity to an amino acid sequence of SEQ ID NO:1289, and a VL having an amino acid sequence having at least 95% identity to an amino acid sequence of SEQ ID NO:1290. In one aspect, provided herein is an antibody that binds TRGV9, comprising a heavy chain having an amino acid sequence having at least 95% identity to an amino acid sequence of SEQ ID NO:1291. In one aspect, provided herein is an antibody that binds TRGV9, comprising a light chain having an amino acid sequence having at least 95% identity to an amino acid sequence of SEQ ID NO:1292. In one aspect, provided herein is an antibody that binds TRGV9, comprising a heavy chain having an amino acid sequence having at least 95% identity to an amino acid sequence of SEQ ID NO:1291, and a light chain having an amino acid sequence having at least 95% identity to an amino acid sequence of SEQ ID NO:1292.

In one aspect, provided herein is an antibody that binds TRGV9, comprising: (i) a VH comprising a VH CDR1, a VH CDR2, and a VH CDR3 having an amino acid sequence of SEQ ID NOs:1293, 1294, and 1295, respectively, and (ii) a VL comprising a VL CDR1, VL CDR2, and VL CDR3 having an amino acid sequence of SEQ ID NOs:1296, 1297, and 1298, respectively. In one aspect, provided herein is an antibody that binds TRGV9, comprising: (i) a VH comprising a VH CDR1, a VH CDR2, and a VH CDR3 having an amino acid sequence of SEQ ID NOs:1299, 1300, and 1301, respectively, and (ii) a VL comprising a VL CDR1, VL CDR2, and VL CDR3 having an amino acid sequence of SEQ ID NOs:1302, 1303, and 1304, respectively. In one aspect, provided herein is an antibody that binds TRGV9, comprising: (i) a VH comprising a VH CDR1, a VH CDR2, and a VH CDR3 having an amino acid sequence of SEQ ID NOs:1305, 1306, and 1307, respectively, and (ii) a VL comprising a VL CDR1, VL CDR2, and VL CDR3 having an amino acid sequence of SEQ ID NOs:1308, 1309, and 1310, respectively. In one aspect, provided herein is an antibody that binds TRGV9, comprising: (i) a VH comprising a VH CDR1, a VH CDR2, and a VH CDR3 having an amino acid sequence of SEQ ID NOs:1311, 1312, and 1313, respectively, and (ii) a VL comprising a VL CDR1, VL CDR2, and VL CDR3 having an amino acid sequence of SEQ ID NOs:1314, 1315, and 1316, respectively. In one aspect, provided herein is an antibody that binds TRGV9, comprising: (i) a VH comprising a VH CDR1, a VH CDR2, and a VH CDR3 having an amino acid sequence of SEQ ID NOs:1317, 1318, and 1319, respectively, and (ii) a VL comprising a VL CDR1, VL CDR2, and VL CDR3 having an amino acid sequence of SEQ ID NOs:1320, 1321, and 1322, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1323; and (ii) a VL comprising a VL CDR1, a VL CDR2, and a VL CDR3 having an amino acid sequence of a VL CDR1, a VL CDR2, and a VL CDR3, respectively, of SEQ ID NO:1324. In one aspect, provided herein is an antibody that binds TRGV9, comprising a VH having an amino acid sequence of SEQ ID NO:1323. In one aspect, provided herein is an antibody that binds TRGV9, comprising a VL having an amino acid sequence of SEQ ID NO:1324. In one aspect, provided herein is an antibody that binds TRGV9, comprising a VH having an amino acid sequence of SEQ ID NO:1323, and a VL having an amino acid sequence of SEQ ID NO:1324. In one aspect, provided herein is an antibody that binds TRGV9, comprising a heavy chain having an amino acid sequence of SEQ ID NO:1325. In one aspect, provided herein is an antibody that binds TRGV9, comprising a light chain having an amino acid sequence of SEQ ID NO:1326. In one aspect, provided herein is an antibody that binds TRGV9, comprising a heavy chain having an amino acid sequence of SEQ ID NO:1325, and a light chain having an amino acid sequence of SEQ ID NO:1326. In one aspect, provided herein is an antibody that binds TRGV9, comprising a VH having an amino acid sequence having at least 95% identity to an amino acid sequence of SEQ ID NO:1323. In one aspect, provided herein is an antibody that binds TRGV9, comprising a VL having an amino acid sequence having at least 95% identity to an amino acid sequence of SEQ ID NO:1324. In one aspect, provided herein is an antibody that binds TRGV9, comprising a VH having an amino acid sequence having at least 95% identity to an amino acid sequence of SEQ ID NO:1323, and a VL having an amino acid sequence having at least 95% identity to an amino acid sequence of SEQ ID NO:1324. In one aspect, provided herein is an antibody that binds TRGV9, comprising a heavy chain having an amino acid sequence having at least 95% identity to an amino acid sequence of SEQ ID NO:1325. In one aspect, provided herein is an antibody that binds TRGV9, comprising a light chain having an amino acid sequence having at least 95% identity to an amino acid sequence of SEQ ID NO:1326. In one aspect, provided herein is an antibody that binds TRGV9, comprising a heavy chain having an amino acid sequence having at least 95% identity to an amino acid sequence of SEQ ID NO:1325, and a light chain having an amino acid sequence having at least 95% identity to an amino acid sequence of SEQ ID NO:1326.

In one aspect, provided herein is an antibody that binds TRGV9, comprising: (i) a VH comprising a VH CDR1, a VH CDR2, and a VH CDR3 having an amino acid sequence of SEQ ID NOs:1327, 1328, and 1329, respectively, and (ii) a VL comprising a VL CDR1, VL CDR2, and VL CDR3 having an amino acid sequence of SEQ ID NOs:1330, 1331, and 1332, respectively. In one aspect, provided herein is an antibody that binds TRGV9, comprising: (i) a VH comprising a VH CDR1, a VH CDR2, and a VH CDR3 having an amino acid sequence of SEQ ID NOs:1333, 1334, and 1335, respectively, and (ii) a VL comprising a VL CDR1, VL CDR2, and VL CDR3 having an amino acid sequence of SEQ ID NOs:1336, 1337, and 1338, respectively. In one aspect, provided herein is an antibody that binds TRGV9, comprising: (i) a VH comprising a VH CDR1, a VH CDR2, and a VH CDR3 having an amino acid sequence of SEQ ID NOs:1339, 1340, and 1341, respectively, and (ii) a VL comprising a VL CDR1, VL CDR2, and VL CDR3 having an amino acid sequence of SEQ ID NOs:1342, 1343, and 1344, respectively. In one aspect, provided herein is an antibody that binds TRGV9, comprising: (i) a VH comprising a VH CDR1, a VH CDR2, and a VH CDR3 having an amino acid sequence of SEQ ID NOs:1345, 1346, and 1347, respectively, and (ii) a VL comprising a VL CDR1, VL CDR2, and VL CDR3 having an amino acid sequence of SEQ ID NOs:1348, 1349, and 1350, respectively. In one aspect, provided herein is an antibody that binds TRGV9, comprising: (i) a VH comprising a VH CDR1, a VH CDR2, and a VH CDR3 having an amino acid sequence of SEQ ID NOs:1351, 1352, and 1353, respectively, and (ii) a VL comprising a VL CDR1, VL CDR2, and VL CDR3 having an amino acid sequence of SEQ ID NOs:1354, 1355, and 1356, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1357; and (ii) a VL comprising a VL CDR1, a VL CDR2, and a VL CDR3 having an amino acid sequence of a VL CDR1, a VL CDR2, and a VL CDR3, respectively, of SEQ ID NO:1358. In one aspect, provided herein is an antibody that binds TRGV9, comprising a VH having an amino acid sequence of SEQ ID NO:1357. In one aspect, provided herein is an antibody that binds TRGV9, comprising a VL having an amino acid sequence of SEQ ID NO:1358. In one aspect, provided herein is an antibody that binds TRGV9, comprising a VH having an amino acid sequence of SEQ ID NO:1357, and a VL having an amino acid sequence of SEQ ID NO:1358. In one aspect, provided herein is an antibody that binds TRGV9, comprising a heavy chain having an amino acid sequence of SEQ ID NO:1359. In one aspect, provided herein is an antibody that binds TRGV9, comprising a light chain having an amino acid sequence of SEQ ID NO:1360. In one aspect, provided herein is an antibody that binds TRGV9, comprising a heavy chain having an amino acid sequence of SEQ ID NO:1359, and a light chain having an amino acid sequence of SEQ ID NO:1360. In one aspect, provided herein is an antibody that binds TRGV9, comprising a VH having an amino acid sequence having at least 95% identity to an amino acid sequence of SEQ ID NO:1357. In one aspect, provided herein is an antibody that binds TRGV9, comprising a VL having an amino acid sequence having at least 95% identity to an amino acid sequence of SEQ ID NO:1358. In one aspect, provided herein is an antibody that binds TRGV9, comprising a VH having an amino acid sequence having at least 95% identity to an amino acid sequence of SEQ ID NO:1357, and a VL having an amino acid sequence having at least 95% identity to an amino acid sequence of SEQ ID NO:1358. In one aspect, provided herein is an antibody that binds TRGV9, comprising a heavy chain having an amino acid sequence having at least 95% identity to an amino acid sequence of SEQ ID NO:1359. In one aspect, provided herein is an antibody that binds TRGV9, comprising a light chain having an amino acid sequence having at least 95% identity to an amino acid sequence of SEQ ID NO:1360. In one aspect, provided herein is an antibody that binds TRGV9, comprising a heavy chain having an amino acid sequence having at least 95% identity to an amino acid sequence of SEQ ID NO:1359, and a light chain having an amino acid sequence having at least 95% identity to an amino acid sequence of SEQ ID NO:1360.

In one aspect, provided herein is an antibody that binds TRGV9, comprising: (i) a VH comprising a VH CDR1, a VH CDR2, and a VH CDR3 having an amino acid sequence of SEQ ID NOs:1361, 1362, and 1363, respectively, and (ii) a VL comprising a VL CDR1, VL CDR2, and VL CDR3 having an amino acid sequence of SEQ ID NOs:1364, 1365, and 1366, respectively. In one aspect, provided herein is an antibody that binds TRGV9, comprising: (i) a VH comprising a VH CDR1, a VH CDR2, and a VH CDR3 having an amino acid sequence of SEQ ID NOs:1367, 1368, and 1369, respectively, and (ii) a VL comprising a VL CDR1, VL CDR2, and VL CDR3 having an amino acid sequence of SEQ ID NOs:1370, 1371, and 1372, respectively. In one aspect, provided herein is an antibody that binds TRGV9, comprising: (i) a VH comprising a VH CDR1, a VH CDR2, and a VH CDR3 having an amino acid sequence of SEQ ID NOs:1373, 1374, and 1375, respectively, and (ii) a VL comprising a VL CDR1, VL CDR2, and VL CDR3 having an amino acid sequence of SEQ ID NOs:1376, 1377, and 1378, respectively. In one aspect, provided herein is an antibody that binds TRGV9, comprising: (i) a VH comprising a VH CDR1, a VH CDR2, and a VH CDR3 having an amino acid sequence of SEQ ID NOs:1379, 1380, and 1381, respectively, and (ii) a VL comprising a VL CDR1, VL CDR2, and VL CDR3 having an amino acid sequence of SEQ ID NOs:1382, 1383, and 1384, respectively. In one aspect, provided herein is an antibody that binds TRGV9, comprising: (i) a VH comprising a VH CDR1, a VH CDR2, and a VH CDR3 having an amino acid sequence of SEQ ID NOs:1385, 1386, and 1387, respectively, and (ii) a VL comprising a VL CDR1, VL CDR2, and VL CDR3 having an amino acid sequence of SEQ ID NOs:1388, 1389, and 1390, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1391; and (ii) a VL comprising a VL CDR1, a VL CDR2, and a VL CDR3 having an amino acid sequence of a VL CDR1, a VL CDR2, and a VL CDR3, respectively, of SEQ ID NO:1392. In one aspect, provided herein is an antibody that binds TRGV9, comprising a VH having an amino acid sequence of SEQ ID NO:1391. In one aspect, provided herein is an antibody that binds TRGV9, comprising a VL having an amino acid sequence of SEQ ID NO:1392. In one aspect, provided herein is an antibody that binds TRGV9, comprising a VH having an amino acid sequence of SEQ ID NO:1391, and a VL having an amino acid sequence of SEQ ID NO:1392. In one aspect, provided herein is an antibody that binds TRGV9, comprising a heavy chain having an amino acid sequence of SEQ ID NO:1393. In one aspect, provided herein is an antibody that binds TRGV9, comprising a light chain having an amino acid sequence of SEQ ID NO:1394. In one aspect, provided herein is an antibody that binds TRGV9, comprising a heavy chain having an amino acid sequence of SEQ ID NO:1393, and a light chain having an amino acid sequence of SEQ ID NO:1394. In one aspect, provided herein is an antibody that binds TRGV9, comprising a VH having an amino acid sequence having at least 95% identity to an amino acid sequence of SEQ ID NO:1391. In one aspect, provided herein is an antibody that binds TRGV9, comprising a VL having an amino acid sequence having at least 95% identity to an amino acid sequence of SEQ ID NO:1392. In one aspect, provided herein is an antibody that binds TRGV9, comprising a VH having an amino acid sequence having at least 95% identity to an amino acid sequence of SEQ ID NO:1391, and a VL having an amino acid sequence having at least 95% identity to an amino acid sequence of SEQ ID NO:1392. In one aspect, provided herein is an antibody that binds TRGV9, comprising a heavy chain having an amino acid sequence having at least 95% identity to an amino acid sequence of SEQ ID NO:1393. In one aspect, provided herein is an antibody that binds TRGV9, comprising a light chain having an amino acid sequence having at least 95% identity to an amino acid sequence of SEQ ID NO:1394. In one aspect, provided herein is an antibody that binds TRGV9, comprising a heavy chain having an amino acid sequence having at least 95% identity to an amino acid sequence of SEQ ID NO:1393, and a light chain having an amino acid sequence having at least 95% identity to an amino acid sequence of SEQ ID NO:1394.

In one aspect, provided herein is an antibody that binds TRGV9, comprising: (i) a VH comprising a VH CDR1, a VH CDR2, and a VH CDR3 having an amino acid sequence of SEQ ID NOs:1395, 1396, and 1397, respectively, and (ii) a VL comprising a VL CDR1, VL CDR2, and VL CDR3 having an amino acid sequence of SEQ ID NOs:1398, 1399, and 1400, respectively. In one aspect, provided herein is an antibody that binds TRGV9, comprising: (i) a VH comprising a VH CDR1, a VH CDR2, and a VH CDR3 having an amino acid sequence of SEQ ID NOs:1401, 1402, and 1403, respectively, and (ii) a VL comprising a VL CDR1, VL CDR2, and VL CDR3 having an amino acid sequence of SEQ ID NOs:1404, 1405, and 1406, respectively. In one aspect, provided herein is an antibody that binds TRGV9, comprising: (i) a VH comprising a VH CDR1, a VH CDR2, and a VH CDR3 having an amino acid sequence of SEQ ID NOs:1407, 1408, and 1409, respectively, and (ii) a VL comprising a VL CDR1, VL CDR2, and VL CDR3 having an amino acid sequence of SEQ ID NOs:1410, 1411, and 1412, respectively. In one aspect, provided herein is an antibody that binds TRGV9, comprising: (i) a VH comprising a VH CDR1, a VH CDR2, and a VH CDR3 having an amino acid sequence of SEQ ID NOs:1413, 1414, and 1415, respectively, and (ii) a VL comprising a VL CDR1, VL CDR2, and VL CDR3 having an amino acid sequence of SEQ ID NOs:1416, 1417, and 1418, respectively. In one aspect, provided herein is an antibody that binds TRGV9, comprising: (i) a VH comprising a VH CDR1, a VH CDR2, and a VH CDR3 having an amino acid sequence of SEQ ID NOs:1419, 1420, and 1421, respectively, and (ii) a VL comprising a VL CDR1, VL CDR2, and VL CDR3 having an amino acid sequence of SEQ ID NOs:1422, 1423, and 1424, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1425; and (ii) a VL comprising a VL CDR1, a VL CDR2, and a VL CDR3 having an amino acid sequence of a VL CDR1, a VL CDR2, and a VL CDR3, respectively, of SEQ ID NO:1426. In one aspect, provided herein is an antibody that binds TRGV9, comprising a VH having an amino acid sequence of SEQ ID NO:1425. In one aspect, provided herein is an antibody that binds TRGV9, comprising a VL having an amino acid sequence of SEQ ID NO:1426. In one aspect, provided herein is an antibody that binds TRGV9, comprising a VH having an amino acid sequence of SEQ ID NO:1425, and a VL having an amino acid sequence of SEQ ID NO:1426. In one aspect, provided herein is an antibody that binds TRGV9, comprising a heavy chain having an amino acid sequence of SEQ ID NO:1427. In one aspect, provided herein is an antibody that binds TRGV9, comprising a light chain having an amino acid sequence of SEQ ID NO:1428. In one aspect, provided herein is an antibody that binds TRGV9, comprising a heavy chain having an amino acid sequence of SEQ ID NO:1427, and a light chain having an amino acid sequence of SEQ ID NO:1428. In one aspect, provided herein is an antibody that binds TRGV9, comprising a VH having an amino acid sequence having at least 95% identity to an amino acid sequence of SEQ ID NO:1425. In one aspect, provided herein is an antibody that binds TRGV9, comprising a VL having an amino acid sequence having at least 95% identity to an amino acid sequence of SEQ ID NO:1426. In one aspect, provided herein is an antibody that binds TRGV9, comprising a VH having an amino acid sequence having at least 95% identity to an amino acid sequence of SEQ ID NO:1425, and a VL having an amino acid sequence having at least 95% identity to an amino acid sequence of SEQ ID NO:1426. In one aspect, provided herein is an antibody that binds TRGV9, comprising a heavy chain having an amino acid sequence having at least 95% identity to an amino acid sequence of SEQ ID NO:1427. In one aspect, provided herein is an antibody that binds TRGV9, comprising a light chain having an amino acid sequence having at least 95% identity to an amino acid sequence of SEQ ID NO:1428. In one aspect, provided herein is an antibody that binds TRGV9, comprising a heavy chain having an amino acid sequence having at least 95% identity to an amino acid sequence of SEQ ID NO:1427, and a light chain having an amino acid sequence having at least 95% identity to an amino acid sequence of SEQ ID NO:1428.

In one aspect, provided herein is an antibody that binds TRGV9, comprising: (i) a VH comprising a VH CDR1, a VH CDR2, and a VH CDR3 having an amino acid sequence of SEQ ID NOs:1429, 1430, and 1431, respectively, and (ii) a VL comprising a VL CDR1, VL CDR2, and VL CDR3 having an amino acid sequence of SEQ ID NOs:1432, 1433, and 1434, respectively. In one aspect, provided herein is an antibody that binds TRGV9, comprising: (i) a VH comprising a VH CDR1, a VH CDR2, and a VH CDR3 having an amino acid sequence of SEQ ID NOs:1435, 1436, and 1437, respectively, and (ii) a VL comprising a VL CDR1, VL CDR2, and VL CDR3 having an amino acid sequence of SEQ ID NOs:1438, 1439, and 1440, respectively. In one aspect, provided herein is an antibody that binds TRGV9, comprising: (i) a VH comprising a VH CDR1, a VH CDR2, and a VH CDR3 having an amino acid sequence of SEQ ID NOs:1441, 1442, and 1443, respectively, and (ii) a VL comprising a VL CDR1, VL CDR2, and VL CDR3 having an amino acid sequence of SEQ ID NOs:1444, 1445, and 1446, respectively. In one aspect, provided herein is an antibody that binds TRGV9, comprising: (i) a VH comprising a VH CDR1, a VH CDR2, and a VH CDR3 having an amino acid sequence of SEQ ID NOs:1447, 1448, and 1449, respectively, and (ii) a VL comprising a VL CDR1, VL CDR2, and VL CDR3 having an amino acid sequence of SEQ ID NOs:1450, 1451, and 1452, respectively. In one aspect, provided herein is an antibody that binds TRGV9, comprising: (i) a VH comprising a VH CDR1, a VH CDR2, and a VH CDR3 having an amino acid sequence of SEQ ID NOs:1453, 1454, and 1455, respectively, and (ii) a VL comprising a VL CDR1, VL CDR2, and VL CDR3 having an amino acid sequence of SEQ ID NOs:1456, 1457, and 1458, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1459; and (ii) a VL comprising a VL CDR1, a VL CDR2, and a VL CDR3 having an amino acid sequence of a VL CDR1, a VL CDR2, and a VL CDR3, respectively, of SEQ ID NO:1460. In one aspect, provided herein is an antibody that binds TRGV9, comprising a VH having an amino acid sequence of SEQ ID NO:1459. In one aspect, provided herein is an antibody that binds TRGV9, comprising a VL having an amino acid sequence of SEQ ID NO:1460. In one aspect, provided herein is an antibody that binds TRGV9, comprising a VH having an amino acid sequence of SEQ ID NO:1459, and a VL having an amino acid sequence of SEQ ID NO:1460. In one aspect, provided herein is an antibody that binds TRGV9, comprising a heavy chain having an amino acid sequence of SEQ ID NO:1461. In one aspect, provided herein is an antibody that binds TRGV9, comprising a light chain having an amino acid sequence of SEQ ID NO:1462. In one aspect, provided herein is an antibody that binds TRGV9, comprising a heavy chain having an amino acid sequence of SEQ ID NO:1461, and a light chain having an amino acid sequence of SEQ ID NO:1462. In one aspect, provided herein is an antibody that binds TRGV9, comprising a VH having an amino acid sequence having at least 95% identity to an amino acid sequence of SEQ ID NO:1459. In one aspect, provided herein is an antibody that binds TRGV9, comprising a VL having an amino acid sequence having at least 95% identity to an amino acid sequence of SEQ ID NO:1460. In one aspect, provided herein is an antibody that binds TRGV9, comprising a VH having an amino acid sequence having at least 95% identity to an amino acid sequence of SEQ ID NO:1459, and a VL having an amino acid sequence having at least 95% identity to an amino acid sequence of SEQ ID NO:1460. In one aspect, provided herein is an antibody that binds TRGV9, comprising a heavy chain having an amino acid sequence having at least 95% identity to an amino acid sequence of SEQ ID NO:1461. In one aspect, provided herein is an antibody that binds TRGV9, comprising a light chain having an amino acid sequence having at least 95% identity to an amino acid sequence of SEQ ID NO:1462. In one aspect, provided herein is an antibody that binds TRGV9, comprising a heavy chain having an amino acid sequence having at least 95% identity to an amino acid sequence of SEQ ID NO:1461, and a light chain having an amino acid sequence having at least 95% identity to an amino acid sequence of SEQ ID NO:1462.

In one aspect, provided herein is an antibody that binds TRGV9, comprising: (i) a VH comprising a VH CDR1, a VH CDR2, and a VH CDR3 having an amino acid sequence of SEQ ID NOs:1463, 1464, and 1465, respectively, and (ii) a VL comprising a VL CDR1, VL CDR2, and VL CDR3 having an amino acid sequence of SEQ ID NOs:1466, 1467, and 1468, respectively. In one aspect, provided herein is an antibody that binds TRGV9, comprising: (i) a VH comprising a VH CDR1, a VH CDR2, and a VH CDR3 having an amino acid sequence of SEQ ID NOs:1469, 1470, and 1471, respectively, and (ii) a VL comprising a VL CDR1, VL CDR2, and VL CDR3 having an amino acid sequence of SEQ ID NOs:1472, 1473, and 1474, respectively. In one aspect, provided herein is an antibody that binds TRGV9, comprising: (i) a VH comprising a VH CDR1, a VH CDR2, and a VH CDR3 having an amino acid sequence of SEQ ID NOs:1475, 1476, and 1477, respectively, and (ii) a VL comprising a VL CDR1, VL CDR2, and VL CDR3 having an amino acid sequence of SEQ ID NOs:1478, 1479, and 1480, respectively. In one aspect, provided herein is an antibody that binds TRGV9, comprising: (i) a VH comprising a VH CDR1, a VH CDR2, and a VH CDR3 having an amino acid sequence of SEQ ID NOs:1481, 1482, and 1483, respectively, and (ii) a VL comprising a VL CDR1, VL CDR2, and VL CDR3 having an amino acid sequence of SEQ ID NOs:1484, 1485, and 1486, respectively. In one aspect, provided herein is an antibody that binds TRGV9, comprising: (i) a VH comprising a VH CDR1, a VH CDR2, and a VH CDR3 having an amino acid sequence of SEQ ID NOs:1487, 1488, and 1489, respectively, and (ii) a VL comprising a VL CDR1, VL CDR2, and VL CDR3 having an amino acid sequence of SEQ ID NOs:1490, 1491, and 1492, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1493; and (ii) a VL comprising a VL CDR1, a VL CDR2, and a VL CDR3 having an amino acid sequence of a VL CDR1, a VL CDR2, and a VL CDR3, respectively, of SEQ ID NO:1494. In one aspect, provided herein is an antibody that binds TRGV9, comprising a VH having an amino acid sequence of SEQ ID NO:1493. In one aspect, provided herein is an antibody that binds TRGV9, comprising a VL having an amino acid sequence of SEQ ID NO:1494. In one aspect, provided herein is an antibody that binds TRGV9, comprising a VH having an amino acid sequence of SEQ ID NO:1493, and a VL having an amino acid sequence of SEQ ID NO:1494. In one aspect, provided herein is an antibody that binds TRGV9, comprising a heavy chain having an amino acid sequence of SEQ ID NO:1495. In one aspect, provided herein is an antibody that binds TRGV9, comprising a light chain having an amino acid sequence of SEQ ID NO:1496. In one aspect, provided herein is an antibody that binds TRGV9, comprising a heavy chain having an amino acid sequence of SEQ ID NO:1495, and a light chain having an amino acid sequence of SEQ ID NO:1496. In one aspect, provided herein is an antibody that binds TRGV9, comprising a VH having an amino acid sequence having at least 95% identity to an amino acid sequence of SEQ ID NO:1493. In one aspect, provided herein is an antibody that binds TRGV9, comprising a VL having an amino acid sequence having at least 95% identity to an amino acid sequence of SEQ ID NO:1494. In one aspect, provided herein is an antibody that binds TRGV9, comprising a VH having an amino acid sequence having at least 95% identity to an amino acid sequence of SEQ ID NO:1493, and a VL having an amino acid sequence having at least 95% identity to an amino acid sequence of SEQ ID NO:1494. In one aspect, provided herein is an antibody that binds TRGV9, comprising a heavy chain having an amino acid sequence having at least 95% identity to an amino acid sequence of SEQ ID NO:1495. In one aspect, provided herein is an antibody that binds TRGV9, comprising a light chain having an amino acid sequence having at least 95% identity to an amino acid sequence of SEQ ID NO:1496. In one aspect, provided herein is an antibody that binds TRGV9, comprising a heavy chain having an amino acid sequence having at least 95% identity to an amino acid sequence of SEQ ID NO:1495, and a light chain having an amino acid sequence having at least 95% identity to an amino acid sequence of SEQ ID NO:1496.

In one aspect, provided herein is an antibody that binds TRGV9, comprising: (i) a VH comprising a VH CDR1, a VH CDR2, and a VH CDR3 having an amino acid sequence of SEQ ID NOs:1497, 1498, and 1499, respectively, and (ii) a VL comprising a VL CDR1, VL CDR2, and VL CDR3 having an amino acid sequence of SEQ ID NOs:1500, 1501, and 1502, respectively. In one aspect, provided herein is an antibody that binds TRGV9, comprising: (i) a VH comprising a VH CDR1, a VH CDR2, and a VH CDR3 having an amino acid sequence of SEQ ID NOs:1503, 1504, and 1505, respectively, and (ii) a VL comprising a VL CDR1, VL CDR2, and VL CDR3 having an amino acid sequence of SEQ ID NOs:1506, 1507, and 1508, respectively. In one aspect, provided herein is an antibody that binds TRGV9, comprising: (i) a VH comprising a VH CDR1, a VH CDR2, and a VH CDR3 having an amino acid sequence of SEQ ID NOs:1509, 1510, and 1511, respectively, and (ii) a VL comprising a VL CDR1, VL CDR2, and VL CDR3 having an amino acid sequence of SEQ ID NOs:1512, 1513, and 1514, respectively. In one aspect, provided herein is an antibody that binds TRGV9, comprising: (i) a VH comprising a VH CDR1, a VH CDR2, and a VH CDR3 having an amino acid sequence of SEQ ID NOs:1515, 1516, and 1517, respectively, and (ii) a VL comprising a VL CDR1, VL CDR2, and VL CDR3 having an amino acid sequence of SEQ ID NOs:1518, 1519, and 1520, respectively. In one aspect, provided herein is an antibody that binds TRGV9, comprising: (i) a VH comprising a VH CDR1, a VH CDR2, and a VH CDR3 having an amino acid sequence of SEQ ID NOs:1521, 1522, and 1523, respectively, and (ii) a VL comprising a VL CDR1, VL CDR2, and VL CDR3 having an amino acid sequence of SEQ ID NOs:1524, 1525, and 1526, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1527; and (ii) a VL comprising a VL CDR1, a VL CDR2, and a VL CDR3 having an amino acid sequence of a VL CDR1, a VL CDR2, and a VL CDR3, respectively, of SEQ ID NO:1528. In one aspect, provided herein is an antibody that binds TRGV9, comprising a VH having an amino acid sequence of SEQ ID NO:1527. In one aspect, provided herein is an antibody that binds TRGV9, comprising a VL having an amino acid sequence of SEQ ID NO:1528. In one aspect, provided herein is an antibody that binds TRGV9, comprising a VH having an amino acid sequence of SEQ ID NO:1527, and a VL having an amino acid sequence of SEQ ID NO:1528. In one aspect, provided herein is an antibody that binds TRGV9, comprising a heavy chain having an amino acid sequence of SEQ ID NO:1529. In one aspect, provided herein is an antibody that binds TRGV9, comprising a light chain having an amino acid sequence of SEQ ID NO:1530. In one aspect, provided herein is an antibody that binds TRGV9, comprising a heavy chain having an amino acid sequence of SEQ ID NO:1529, and a light chain having an amino acid sequence of SEQ ID NO:1530. In one aspect, provided herein is an antibody that binds TRGV9, comprising a VH having an amino acid sequence having at least 95% identity to an amino acid sequence of SEQ ID NO:1527. In one aspect, provided herein is an antibody that binds TRGV9, comprising a VL having an amino acid sequence having at least 95% identity to an amino acid sequence of SEQ ID NO:1528. In one aspect, provided herein is an antibody that binds TRGV9, comprising a VH having an amino acid sequence having at least 95% identity to an amino acid sequence of SEQ ID NO:1527, and a VL having an amino acid sequence having at least 95% identity to an amino acid sequence of SEQ ID NO:1528. In one aspect, provided herein is an antibody that binds TRGV9, comprising a heavy chain having an amino acid sequence having at least 95% identity to an amino acid sequence of SEQ ID NO:1529. In one aspect, provided herein is an antibody that binds TRGV9, comprising a light chain having an amino acid sequence having at least 95% identity to an amino acid sequence of SEQ ID NO:1530. In one aspect, provided herein is an antibody that binds TRGV9, comprising a heavy chain having an amino acid sequence having at least 95% identity to an amino acid sequence of SEQ ID NO:1529, and a light chain having an amino acid sequence having at least 95% identity to an amino acid sequence of SEQ ID NO:1530.

In one aspect, provided herein is an antibody that binds TRGV9, comprising: (i) a VH comprising a VH CDR1, a VH CDR2, and a VH CDR3 having an amino acid sequence of SEQ ID NOs:1531, 1532, and 1533, respectively, and (ii) a VL comprising a VL CDR1, VL CDR2, and VL CDR3 having an amino acid sequence of SEQ ID NOs:1534, 1535, and 1536, respectively. In one aspect, provided herein is an antibody that binds TRGV9, comprising: (i) a VH comprising a VH CDR1, a VH CDR2, and a VH CDR3 having an amino acid sequence of SEQ ID NOs:1537, 1538, and 1539, respectively, and (ii) a VL comprising a VL CDR1, VL CDR2, and VL CDR3 having an amino acid sequence of SEQ ID NOs:1540, 1541, and 1542, respectively. In one aspect, provided herein is an antibody that binds TRGV9, comprising: (i) a VH comprising a VH CDR1, a VH CDR2, and a VH CDR3 having an amino acid sequence of SEQ ID NOs:1543, 1544, and 1545, respectively, and (ii) a VL comprising a VL CDR1, VL CDR2, and VL CDR3 having an amino acid sequence of SEQ ID NOs:1546, 1547, and 1548, respectively. In one aspect, provided herein is an antibody that binds TRGV9, comprising: (i) a VH comprising a VH CDR1, a VH CDR2, and a VH CDR3 having an amino acid sequence of SEQ ID NOs:1549, 1550, and 1551, respectively, and (ii) a VL comprising a VL CDR1, VL CDR2, and VL CDR3 having an amino acid sequence of SEQ ID NOs:1552, 1553, and 1554, respectively. In one aspect, provided herein is an antibody that binds TRGV9, comprising: (i) a VH comprising a VH CDR1, a VH CDR2, and a VH CDR3 having an amino acid sequence of SEQ ID NOs:1555, 1556, and 1557, respectively, and (ii) a VL comprising a VL CDR1, VL CDR2, and VL CDR3 having an amino acid sequence of SEQ ID NOs:1558, 1559, and 1560, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1561; and (ii) a VL comprising a VL CDR1, a VL CDR2, and a VL CDR3 having an amino acid sequence of a VL CDR1, a VL CDR2, and a VL CDR3, respectively, of SEQ ID NO:1562. In one aspect, provided herein is an antibody that binds TRGV9, comprising a VH having an amino acid sequence of SEQ ID NO:1561. In one aspect, provided herein is an antibody that binds TRGV9, comprising a VL having an amino acid sequence of SEQ ID NO:1562. In one aspect, provided herein is an antibody that binds TRGV9, comprising a VH having an amino acid sequence of SEQ ID NO:1561, and a VL having an amino acid sequence of SEQ ID NO:1562. In one aspect, provided herein is an antibody that binds TRGV9, comprising a heavy chain having an amino acid sequence of SEQ ID NO:1563. In one aspect, provided herein is an antibody that binds TRGV9, comprising a light chain having an amino acid sequence of SEQ ID NO:1564. In one aspect, provided herein is an antibody that binds TRGV9, comprising a heavy chain having an amino acid sequence of SEQ ID NO:1563, and a light chain having an amino acid sequence of SEQ ID NO:1564. In one aspect, provided herein is an antibody that binds TRGV9, comprising a VH having an amino acid sequence having at least 95% identity to an amino acid sequence of SEQ ID NO:1561. In one aspect, provided herein is an antibody that binds TRGV9, comprising a VL having an amino acid sequence having at least 95% identity to an amino acid sequence of SEQ ID NO:1562. In one aspect, provided herein is an antibody that binds TRGV9, comprising a VH having an amino acid sequence having at least 95% identity to an amino acid sequence of SEQ ID NO:1561, and a VL having an amino acid sequence having at least 95% identity to an amino acid sequence of SEQ ID NO:1562. In one aspect, provided herein is an antibody that binds TRGV9, comprising a heavy chain having an amino acid sequence having at least 95% identity to an amino acid sequence of SEQ ID NO:1563. In one aspect, provided herein is an antibody that binds TRGV9, comprising a light chain having an amino acid sequence having at least 95% identity to an amino acid sequence of SEQ ID NO:1564. In one aspect, provided herein is an antibody that binds TRGV9, comprising a heavy chain having an amino acid sequence having at least 95% identity to an amino acid sequence of SEQ ID NO:1563, and a light chain having an amino acid sequence having at least 95% identity to an amino acid sequence of SEQ ID NO:1564.

In one aspect, provided herein is an antibody that binds TRGV9, comprising: (i) a VH comprising a VH CDR1, a VH CDR2, and a VH CDR3 having an amino acid sequence of SEQ ID NOs:1565, 1566, and 1567, respectively, and (ii) a VL comprising a VL CDR1, VL CDR2, and VL CDR3 having an amino acid sequence of SEQ ID NOs:1568, 1569, and 1570, respectively. In one aspect, provided herein is an antibody that binds TRGV9, comprising: (i) a VH comprising a VH CDR1, a VH CDR2, and a VH CDR3 having an amino acid sequence of SEQ ID NOs:1571, 1572, and 1573, respectively, and (ii) a VL comprising a VL CDR1, VL CDR2, and VL CDR3 having an amino acid sequence of SEQ ID NOs:1574, 1575, and 1576, respectively. In one aspect, provided herein is an antibody that binds TRGV9, comprising: (i) a VH comprising a VH CDR1, a VH CDR2, and a VH CDR3 having an amino acid sequence of SEQ ID NOs:1577, 1578, and 1579, respectively, and (ii) a VL comprising a VL CDR1, VL CDR2, and VL CDR3 having an amino acid sequence of SEQ ID NOs:1580, 1581, and 1582, respectively. In one aspect, provided herein is an antibody that binds TRGV9, comprising: (i) a VH comprising a VH CDR1, a VH CDR2, and a VH CDR3 having an amino acid sequence of SEQ ID NOs:1583, 1584, and 1585, respectively, and (ii) a VL comprising a VL CDR1, VL CDR2, and VL CDR3 having an amino acid sequence of SEQ ID NOs:1586, 1587, and 1588, respectively. In one aspect, provided herein is an antibody that binds TRGV9, comprising: (i) a VH comprising a VH CDR1, a VH CDR2, and a VH CDR3 having an amino acid sequence of SEQ ID NOs:1589, 1590, and 1591, respectively, and (ii) a VL comprising a VL CDR1, VL CDR2, and VL CDR3 having an amino acid sequence of SEQ ID NOs:1592, 1593, and 1594, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1595; and (ii) a VL comprising a VL CDR1, a VL CDR2, and a VL CDR3 having an amino acid sequence of a VL CDR1, a VL CDR2, and a VL CDR3, respectively, of SEQ ID NO:1596. In one aspect, provided herein is an antibody that binds TRGV9, comprising a VH having an amino acid sequence of SEQ ID NO:1595. In one aspect, provided herein is an antibody that binds TRGV9, comprising a VL having an amino acid sequence of SEQ ID NO:1596. In one aspect, provided herein is an antibody that binds TRGV9, comprising a VH having an amino acid sequence of SEQ ID NO:1595, and a VL having an amino acid sequence of SEQ ID NO:1596. In one aspect, provided herein is an antibody that binds TRGV9, comprising a heavy chain having an amino acid sequence of SEQ ID NO:1597. In one aspect, provided herein is an antibody that binds TRGV9, comprising a light chain having an amino acid sequence of SEQ ID NO:1598. In one aspect, provided herein is an antibody that binds TRGV9, comprising a heavy chain having an amino acid sequence of SEQ ID NO:1597, and a light chain having an amino acid sequence of SEQ ID NO:1598. In one aspect, provided herein is an antibody that binds TRGV9, comprising a VH having an amino acid sequence having at least 95% identity to an amino acid sequence of SEQ ID NO:1595. In one aspect, provided herein is an antibody that binds TRGV9, comprising a VL having an amino acid sequence having at least 95% identity to an amino acid sequence of SEQ ID NO:1596. In one aspect, provided herein is an antibody that binds TRGV9, comprising a VH having an amino acid sequence having at least 95% identity to an amino acid sequence of SEQ ID NO:1595, and a VL having an amino acid sequence having at least 95% identity to an amino acid sequence of SEQ ID NO:1596. In one aspect, provided herein is an antibody that binds TRGV9, comprising a heavy chain having an amino acid sequence having at least 95% identity to an amino acid sequence of SEQ ID NO:1597. In one aspect, provided herein is an antibody that binds TRGV9, comprising a light chain having an amino acid sequence having at least 95% identity to an amino acid sequence of SEQ ID NO:1598. In one aspect, provided herein is an antibody that binds TRGV9, comprising a heavy chain having an amino acid sequence having at least 95% identity to an amino acid sequence of SEQ ID NO:1597, and a light chain having an amino acid sequence having at least 95% identity to an amino acid sequence of SEQ ID NO:1598.

In one aspect, provided herein is an antibody that binds TRGV9, comprising: (i) a VH comprising a VH CDR1, a VH CDR2, and a VH CDR3 having an amino acid sequence of SEQ ID NOs:1599, 1600, and 1601, respectively, and (ii) a VL comprising a VL CDR1, VL CDR2, and VL CDR3 having an amino acid sequence of SEQ ID NOs:1602, 1603, and 1604, respectively. In one aspect, provided herein is an antibody that binds TRGV9, comprising: (i) a VH comprising a VH CDR1, a VH CDR2, and a VH CDR3 having an amino acid sequence of SEQ ID NOs:1605, 1606, and 1607, respectively, and (ii) a VL comprising a VL CDR1, VL CDR2, and VL CDR3 having an amino acid sequence of SEQ ID NOs:1608, 1609, and 1610, respectively. In one aspect, provided herein is an antibody that binds TRGV9, comprising: (i) a VH comprising a VH CDR1, a VH CDR2, and a VH CDR3 having an amino acid sequence of SEQ ID NOs:1611, 1612, and 1613, respectively, and (ii) a VL comprising a VL CDR1, VL CDR2, and VL CDR3 having an amino acid sequence of SEQ ID NOs:1614, 1615, and 1616, respectively. In one aspect, provided herein is an antibody that binds TRGV9, comprising: (i) a VH comprising a VH CDR1, a VH CDR2, and a VH CDR3 having an amino acid sequence of SEQ ID NOs:1617, 1618, and 1619, respectively, and (ii) a VL comprising a VL CDR1, VL CDR2, and VL CDR3 having an amino acid sequence of SEQ ID NOs:1620, 1621, and 1622, respectively. In one aspect, provided herein is an antibody that binds TRGV9, comprising: (i) a VH comprising a VH CDR1, a VH CDR2, and a VH CDR3 having an amino acid sequence of SEQ ID NOs:1623, 1624, and 1625, respectively, and (ii) a VL comprising a VL CDR1, VL CDR2, and VL CDR3 having an amino acid sequence of SEQ ID NOs:1626, 1627, and 1628, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1629; and (ii) a VL comprising a VL CDR1, a VL CDR2, and a VL CDR3 having an amino acid sequence of a VL CDR1, a VL CDR2, and a VL CDR3, respectively, of SEQ ID NO:1630. In one aspect, provided herein is an antibody that binds TRGV9, comprising a VH having an amino acid sequence of SEQ ID NO:1629. In one aspect, provided herein is an antibody that binds TRGV9, comprising a VL having an amino acid sequence of SEQ ID NO:1630. In one aspect, provided herein is an antibody that binds TRGV9, comprising a VH having an amino acid sequence of SEQ ID NO:1629, and a VL having an amino acid sequence of SEQ ID NO:1630. In one aspect, provided herein is an antibody that binds TRGV9, comprising a heavy chain having an amino acid sequence of SEQ ID NO:1631. In one aspect, provided herein is an antibody that binds TRGV9, comprising a light chain having an amino acid sequence of SEQ ID NO:1632. In one aspect, provided herein is an antibody that binds TRGV9, comprising a heavy chain having an amino acid sequence of SEQ ID NO:1631, and a light chain having an amino acid sequence of SEQ ID NO:1632. In one aspect, provided herein is an antibody that binds TRGV9, comprising a VH having an amino acid sequence having at least 95% identity to an amino acid sequence of SEQ ID NO:1629. In one aspect, provided herein is an antibody that binds TRGV9, comprising a VL having an amino acid sequence having at least 95% identity to an amino acid sequence of SEQ ID NO:1630. In one aspect, provided herein is an antibody that binds TRGV9, comprising a VH having an amino acid sequence having at least 95% identity to an amino acid sequence of SEQ ID NO:1629, and a VL having an amino acid sequence having at least 95% identity to an amino acid sequence of SEQ ID NO:1630. In one aspect, provided herein is an antibody that binds TRGV9, comprising a heavy chain having an amino acid sequence having at least 95% identity to an amino acid sequence of SEQ ID NO:1631. In one aspect, provided herein is an antibody that binds TRGV9, comprising a light chain having an amino acid sequence having at least 95% identity to an amino acid sequence of SEQ ID NO:1632. In one aspect, provided herein is an antibody that binds TRGV9, comprising a heavy chain having an amino acid sequence having at least 95% identity to an amino acid sequence of SEQ ID NO:1631, and a light chain having an amino acid sequence having at least 95% identity to an amino acid sequence of SEQ ID NO:1632.

In one aspect, provided herein is an antibody that binds TRGV9, comprising: (i) a VH comprising a VH CDR1, a VH CDR2, and a VH CDR3 having an amino acid sequence of SEQ ID NOs:1633, 1634, and 1635, respectively, and (ii) a VL comprising a VL CDR1, VL CDR2, and VL CDR3 having an amino acid sequence of SEQ ID NOs:1636, 1637, and 1638, respectively. In one aspect, provided herein is an antibody that binds TRGV9, comprising: (i) a VH comprising a VH CDR1, a VH CDR2, and a VH CDR3 having an amino acid sequence of SEQ ID NOs:1639, 1640, and 1641, respectively, and (ii) a VL comprising a VL CDR1, VL CDR2, and VL CDR3 having an amino acid sequence of SEQ ID NOs:1642, 1643, and 1644, respectively. In one aspect, provided herein is an antibody that binds TRGV9, comprising: (i) a VH comprising a VH CDR1, a VH CDR2, and a VH CDR3 having an amino acid sequence of SEQ ID NOs:1645, 1646, and 1647, respectively, and (ii) a VL comprising a VL CDR1, VL CDR2, and VL CDR3 having an amino acid sequence of SEQ ID NOs:1648, 1649, and 1650, respectively. In one aspect, provided herein is an antibody that binds TRGV9, comprising: (i) a VH comprising a VH CDR1, a VH CDR2, and a VH CDR3 having an amino acid sequence of SEQ ID NOs:1651, 1652, and 1653, respectively, and (ii) a VL comprising a VL CDR1, VL CDR2, and VL CDR3 having an amino acid sequence of SEQ ID NOs:1654, 1655, and 1656, respectively. In one aspect, provided herein is an antibody that binds TRGV9, comprising: (i) a VH comprising a VH CDR1, a VH CDR2, and a VH CDR3 having an amino acid sequence of SEQ ID NOs:1657, 1658, and 1659, respectively, and (ii) a VL comprising a VL CDR1, VL CDR2, and VL CDR3 having an amino acid sequence of SEQ ID NOs:1660, 1661, and 1662, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1663; and (ii) a VL comprising a VL CDR1, a VL CDR2, and a VL CDR3 having an amino acid sequence of a VL CDR1, a VL CDR2, and a VL CDR3, respectively, of SEQ ID NO:1664. In one aspect, provided herein is an antibody that binds TRGV9, comprising a VH having an amino acid sequence of SEQ ID NO:1663. In one aspect, provided herein is an antibody that binds TRGV9, comprising a VL having an amino acid sequence of SEQ ID NO:1664. In one aspect, provided herein is an antibody that binds TRGV9, comprising a VH having an amino acid sequence of SEQ ID NO:1663, and a VL having an amino acid sequence of SEQ ID NO:1664. In one aspect, provided herein is an antibody that binds TRGV9, comprising a heavy chain having an amino acid sequence of SEQ ID NO:1665. In one aspect, provided herein is an antibody that binds TRGV9, comprising a light chain having an amino acid sequence of SEQ ID NO:1666. In one aspect, provided herein is an antibody that binds TRGV9, comprising a heavy chain having an amino acid sequence of SEQ ID NO:1665, and a light chain having an amino acid sequence of SEQ ID NO:1666. In one aspect, provided herein is an antibody that binds TRGV9, comprising a VH having an amino acid sequence having at least 95% identity to an amino acid sequence of SEQ ID NO:1663. In one aspect, provided herein is an antibody that binds TRGV9, comprising a VL having an amino acid sequence having at least 95% identity to an amino acid sequence of SEQ ID NO:1664. In one aspect, provided herein is an antibody that binds TRGV9, comprising a VH having an amino acid sequence having at least 95% identity to an amino acid sequence of SEQ ID NO:1663, and a VL having an amino acid sequence having at least 95% identity to an amino acid sequence of SEQ ID NO:1664. In one aspect, provided herein is an antibody that binds TRGV9, comprising a heavy chain having an amino acid sequence having at least 95% identity to an amino acid sequence of SEQ ID NO:1665. In one aspect, provided herein is an antibody that binds TRGV9, comprising a light chain having an amino acid sequence having at least 95% identity to an amino acid sequence of SEQ ID NO:1666. In one aspect, provided herein is an antibody that binds TRGV9, comprising a heavy chain having an amino acid sequence having at least 95% identity to an amino acid sequence of SEQ ID NO:1665, and a light chain having an amino acid sequence having at least 95% identity to an amino acid sequence of SEQ ID NO:1666.

In one aspect, provided herein is an antibody that binds TRGV9, comprising: (i) a VH comprising a VH CDR1, a VH CDR2, and a VH CDR3 having an amino acid sequence of SEQ ID NOs:1667, 1668, and 1669, respectively, and (ii) a VL comprising a VL CDR1, VL CDR2, and VL CDR3 having an amino acid sequence of SEQ ID NOs:1670, 1671, and 1672, respectively. In one aspect, provided herein is an antibody that binds TRGV9, comprising: (i) a VH comprising a VH CDR1, a VH CDR2, and a VH CDR3 having an amino acid sequence of SEQ ID NOs:1673, 1674, and 1675, respectively, and (ii) a VL comprising a VL CDR1, VL CDR2, and VL CDR3 having an amino acid sequence of SEQ ID NOs:1676, 1677, and 1678, respectively. In one aspect, provided herein is an antibody that binds TRGV9, comprising: (i) a VH comprising a VH CDR1, a VH CDR2, and a VH CDR3 having an amino acid sequence of SEQ ID NOs:1679, 1680, and 1681, respectively, and (ii) a VL comprising a VL CDR1, VL CDR2, and VL CDR3 having an amino acid sequence of SEQ ID NOs:1682, 1683, and 1684, respectively. In one aspect, provided herein is an antibody that binds TRGV9, comprising: (i) a VH comprising a VH CDR1, a VH CDR2, and a VH CDR3 having an amino acid sequence of SEQ ID NOs:1685, 1686, and 1687, respectively, and (ii) a VL comprising a VL CDR1, VL CDR2, and VL CDR3 having an amino acid sequence of SEQ ID NOs:1688, 1689, and 1690, respectively. In one aspect, provided herein is an antibody that binds TRGV9, comprising: (i) a VH comprising a VH CDR1, a VH CDR2, and a VH CDR3 having an amino acid sequence of SEQ ID NOs:1691, 1692, and 1693, respectively, and (ii) a VL comprising a VL CDR1, VL CDR2, and VL CDR3 having an amino acid sequence of SEQ ID NOs:1694, 1695, and 1696, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1697; and (ii) a VL comprising a VL CDR1, a VL CDR2, and a VL CDR3 having an amino acid sequence of a VL CDR1, a VL CDR2, and a VL CDR3, respectively, of SEQ ID NO:1698. In one aspect, provided herein is an antibody that binds TRGV9, comprising a VH having an amino acid sequence of SEQ ID NO:1697. In one aspect, provided herein is an antibody that binds TRGV9, comprising a VL having an amino acid sequence of SEQ ID NO:1698. In one aspect, provided herein is an antibody that binds TRGV9, comprising a VH having an amino acid sequence of SEQ ID NO:1697, and a VL having an amino acid sequence of SEQ ID NO:1698. In one aspect, provided herein is an antibody that binds TRGV9, comprising a heavy chain having an amino acid sequence of SEQ ID NO:1699. In one aspect, provided herein is an antibody that binds TRGV9, comprising a light chain having an amino acid sequence of SEQ ID NO:1700. In one aspect, provided herein is an antibody that binds TRGV9, comprising a heavy chain having an amino acid sequence of SEQ ID NO:1699, and a light chain having an amino acid sequence of SEQ ID NO:1700. In one aspect, provided herein is an antibody that binds TRGV9, comprising a VH having an amino acid sequence having at least 95% identity to an amino acid sequence of SEQ ID NO:1697. In one aspect, provided herein is an antibody that binds TRGV9, comprising a VL having an amino acid sequence having at least 95% identity to an amino acid sequence of SEQ ID NO:1698. In one aspect, provided herein is an antibody that binds TRGV9, comprising a VH having an amino acid sequence having at least 95% identity to an amino acid sequence of SEQ ID NO:1697, and a VL having an amino acid sequence having at least 95% identity to an amino acid sequence of SEQ ID NO:1698. In one aspect, provided herein is an antibody that binds TRGV9, comprising a heavy chain having an amino acid sequence having at least 95% identity to an amino acid sequence of SEQ ID NO:1699. In one aspect, provided herein is an antibody that binds TRGV9, comprising a light chain having an amino acid sequence having at least 95% identity to an amino acid sequence of SEQ ID NO:1700. In one aspect, provided herein is an antibody that binds TRGV9, comprising a heavy chain having an amino acid sequence having at least 95% identity to an amino acid sequence of SEQ ID NO:1699, and a light chain having an amino acid sequence having at least 95% identity to an amino acid sequence of SEQ ID NO:1700.

In one aspect, provided herein is an antibody that binds TRGV9, comprising: (i) a VH comprising a VH CDR1, a VH CDR2, and a VH CDR3 having an amino acid sequence of SEQ ID NOs:1701, 1702, and 1703, respectively, and (ii) a VL comprising a VL CDR1, VL CDR2, and VL CDR3 having an amino acid sequence of SEQ ID NOs:1704, 1705, and 1706, respectively. In one aspect, provided herein is an antibody that binds TRGV9, comprising: (i) a VH comprising a VH CDR1, a VH CDR2, and a VH CDR3 having an amino acid sequence of SEQ ID NOs:1707, 1708, and 1709, respectively, and (ii) a VL comprising a VL CDR1, VL CDR2, and VL CDR3 having an amino acid sequence of SEQ ID NOs:1710, 1711, and 1712, respectively. In one aspect, provided herein is an antibody that binds TRGV9, comprising: (i) a VH comprising a VH CDR1, a VH CDR2, and a VH CDR3 having an amino acid sequence of SEQ ID NOs:1713, 1714, and 1715, respectively, and (ii) a VL comprising a VL CDR1, VL CDR2, and VL CDR3 having an amino acid sequence of SEQ ID NOs:1716, 1717, and 1718, respectively. In one aspect, provided herein is an antibody that binds TRGV9, comprising: (i) a VH comprising a VH CDR1, a VH CDR2, and a VH CDR3 having an amino acid sequence of SEQ ID NOs:1719, 1720, and 1721, respectively, and (ii) a VL comprising a VL CDR1, VL CDR2, and VL CDR3 having an amino acid sequence of SEQ ID NOs:1722, 1723, and 1724, respectively. In one aspect, provided herein is an antibody that binds TRGV9, comprising: (i) a VH comprising a VH CDR1, a VH CDR2, and a VH CDR3 having an amino acid sequence of SEQ ID NOs:1725, 1726, and 1727, respectively, and (ii) a VL comprising a VL CDR1, VL CDR2, and VL CDR3 having an amino acid sequence of SEQ ID NOs:1728, 1729, and 1730, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1731; and (ii) a VL comprising a VL CDR1, a VL CDR2, and a VL CDR3 having an amino acid sequence of a VL CDR1, a VL CDR2, and a VL CDR3, respectively, of SEQ ID NO:1732. In one aspect, provided herein is an antibody that binds TRGV9, comprising a VH having an amino acid sequence of SEQ ID NO:1731. In one aspect, provided herein is an antibody that binds TRGV9, comprising a VL having an amino acid sequence of SEQ ID NO:1732. In one aspect, provided herein is an antibody that binds TRGV9, comprising a VH having an amino acid sequence of SEQ ID NO:1731, and a VL having an amino acid sequence of SEQ ID NO:1732. In one aspect, provided herein is an antibody that binds TRGV9, comprising a heavy chain having an amino acid sequence of SEQ ID NO:1733. In one aspect, provided herein is an antibody that binds TRGV9, comprising a light chain having an amino acid sequence of SEQ ID NO:1734. In one aspect, provided herein is an antibody that binds TRGV9, comprising a heavy chain having an amino acid sequence of SEQ ID NO:1733, and a light chain having an amino acid sequence of SEQ ID NO:1734. In one aspect, provided herein is an antibody that binds TRGV9, comprising a VH having an amino acid sequence having at least 95% identity to an amino acid sequence of SEQ ID NO:1731. In one aspect, provided herein is an antibody that binds TRGV9, comprising a VL having an amino acid sequence having at least 95% identity to an amino acid sequence of SEQ ID NO:1732. In one aspect, provided herein is an antibody that binds TRGV9, comprising a VH having an amino acid sequence having at least 95% identity to an amino acid sequence of SEQ ID NO:1731, and a VL having an amino acid sequence having at least 95% identity to an amino acid sequence of SEQ ID NO:1732. In one aspect, provided herein is an antibody that binds TRGV9, comprising a heavy chain having an amino acid sequence having at least 95% identity to an amino acid sequence of SEQ ID NO:1733. In one aspect, provided herein is an antibody that binds TRGV9, comprising a light chain having an amino acid sequence having at least 95% identity to an amino acid sequence of SEQ ID NO:1734. In one aspect, provided herein is an antibody that binds TRGV9, comprising a heavy chain having an amino acid sequence having at least 95% identity to an amino acid sequence of SEQ ID NO:1733, and a light chain having an amino acid sequence having at least 95% identity to an amino acid sequence of SEQ ID NO:1734.

In one aspect, provided herein is an antibody that binds TRGV9, comprising: (i) a VH comprising a VH CDR1, a VH CDR2, and a VH CDR3 having an amino acid sequence of SEQ ID NOs:1735, 1736, and 1737, respectively, and (ii) a VL comprising a VL CDR1, VL CDR2, and VL CDR3 having an amino acid sequence of SEQ ID NOs:1738, 1739, and 1740, respectively. In one aspect, provided herein is an antibody that binds TRGV9, comprising: (i) a VH comprising a VH CDR1, a VH CDR2, and a VH CDR3 having an amino acid sequence of SEQ ID NOs:1741, 1742, and 1743, respectively, and (ii) a VL comprising a VL CDR1, VL CDR2, and VL CDR3 having an amino acid sequence of SEQ ID NOs:1744, 1745, and 1746, respectively. In one aspect, provided herein is an antibody that binds TRGV9, comprising: (i) a VH comprising a VH CDR1, a VH CDR2, and a VH CDR3 having an amino acid sequence of SEQ ID NOs:1747, 1748, and 1749, respectively, and (ii) a VL comprising a VL CDR1, VL CDR2, and VL CDR3 having an amino acid sequence of SEQ ID NOs:1750, 1751, and 1752, respectively. In one aspect, provided herein is an antibody that binds TRGV9, comprising: (i) a VH comprising a VH CDR1, a VH CDR2, and a VH CDR3 having an amino acid sequence of SEQ ID NOs:1753, 1754, and 1755, respectively, and (ii) a VL comprising a VL CDR1, VL CDR2, and VL CDR3 having an amino acid sequence of SEQ ID NOs:1756, 1757, and 1758, respectively. In one aspect, provided herein is an antibody that binds TRGV9, comprising: (i) a VH comprising a VH CDR1, a VH CDR2, and a VH CDR3 having an amino acid sequence of SEQ ID NOs:1759, 1760, and 1761, respectively, and (ii) a VL comprising a VL CDR1, VL CDR2, and VL CDR3 having an amino acid sequence of SEQ ID NOs:1762, 1763, and 1764, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1765; and (ii) a VL comprising a VL CDR1, a VL CDR2, and a VL CDR3 having an amino acid sequence of a VL CDR1, a VL CDR2, and a VL CDR3, respectively, of SEQ ID NO:1766. In one aspect, provided herein is an antibody that binds TRGV9, comprising a VH having an amino acid sequence of SEQ ID NO:1765. In one aspect, provided herein is an antibody that binds TRGV9, comprising a VL having an amino acid sequence of SEQ ID NO:1766. In one aspect, provided herein is an antibody that binds TRGV9, comprising a VH having an amino acid sequence of SEQ ID NO:1765, and a VL having an amino acid sequence of SEQ ID NO:1766. In one aspect, provided herein is an antibody that binds TRGV9, comprising a heavy chain having an amino acid sequence of SEQ ID NO:1767. In one aspect, provided herein is an antibody that binds TRGV9, comprising a light chain having an amino acid sequence of SEQ ID NO:1768. In one aspect, provided herein is an antibody that binds TRGV9, comprising a heavy chain having an amino acid sequence of SEQ ID NO:1767, and a light chain having an amino acid sequence of SEQ ID NO:1768. In one aspect, provided herein is an antibody that binds TRGV9, comprising a VH having an amino acid sequence having at least 95% identity to an amino acid sequence of SEQ ID NO:1765. In one aspect, provided herein is an antibody that binds TRGV9, comprising a VL having an amino acid sequence having at least 95% identity to an amino acid sequence of SEQ ID NO:1766. In one aspect, provided herein is an antibody that binds TRGV9, comprising a VH having an amino acid sequence having at least 95% identity to an amino acid sequence of SEQ ID NO:1765, and a VL having an amino acid sequence having at least 95% identity to an amino acid sequence of SEQ ID NO:1766. In one aspect, provided herein is an antibody that binds TRGV9, comprising a heavy chain having an amino acid sequence having at least 95% identity to an amino acid sequence of SEQ ID NO:1767. In one aspect, provided herein is an antibody that binds TRGV9, comprising a light chain having an amino acid sequence having at least 95% identity to an amino acid sequence of SEQ ID NO:1768. In one aspect, provided herein is an antibody that binds TRGV9, comprising a heavy chain having an amino acid sequence having at least 95% identity to an amino acid sequence of SEQ ID NO:1767, and a light chain having an amino acid sequence having at least 95% identity to an amino acid sequence of SEQ ID NO:1768.

In one aspect, provided herein is an antibody that binds TRGV9, comprising: (i) a VH comprising a VH CDR1, a VH CDR2, and a VH CDR3 having an amino acid sequence of SEQ ID NOs:1769, 1770, and 1771, respectively, and (ii) a VL comprising a VL CDR1, VL CDR2, and VL CDR3 having an amino acid sequence of SEQ ID NOs:1772, 1773, and 1774, respectively. In one aspect, provided herein is an antibody that binds TRGV9, comprising: (i) a VH comprising a VH CDR1, a VH CDR2, and a VH CDR3 having an amino acid sequence of SEQ ID NOs:1775, 1776, and 1777, respectively, and (ii) a VL comprising a VL CDR1, VL CDR2, and VL CDR3 having an amino acid sequence of SEQ ID NOs:1778, 1779, and 1780, respectively. In one aspect, provided herein is an antibody that binds TRGV9, comprising: (i) a VH comprising a VH CDR1, a VH CDR2, and a VH CDR3 having an amino acid sequence of SEQ ID NOs:1781, 1782, and 1783, respectively, and (ii) a VL comprising a VL CDR1, VL CDR2, and VL CDR3 having an amino acid sequence of SEQ ID NOs:1784, 1785, and 1786, respectively. In one aspect, provided herein is an antibody that binds TRGV9, comprising: (i) a VH comprising a VH CDR1, a VH CDR2, and a VH CDR3 having an amino acid sequence of SEQ ID NOs:1787, 1788, and 1789, respectively, and (ii) a VL comprising a VL CDR1, VL CDR2, and VL CDR3 having an amino acid sequence of SEQ ID NOs:1790, 1791, and 1792, respectively. In one aspect, provided herein is an antibody that binds TRGV9, comprising: (i) a VH comprising a VH CDR1, a VH CDR2, and a VH CDR3 having an amino acid sequence of SEQ ID NOs:1793, 1794, and 1795, respectively, and (ii) a VL comprising a VL CDR1, VL CDR2, and VL CDR3 having an amino acid sequence of SEQ ID NOs:1796, 1797, and 1798, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1799; and (ii) a VL comprising a VL CDR1, a VL CDR2, and a VL CDR3 having an amino acid sequence of a VL CDR1, a VL CDR2, and a VL CDR3, respectively, of SEQ ID NO:1800. In one aspect, provided herein is an antibody that binds TRGV9, comprising a VH having an amino acid sequence of SEQ ID NO:1799. In one aspect, provided herein is an antibody that binds TRGV9, comprising a VL having an amino acid sequence of SEQ ID NO:1800. In one aspect, provided herein is an antibody that binds TRGV9, comprising a VH having an amino acid sequence of SEQ ID NO:1799, and a VL having an amino acid sequence of SEQ ID NO:1800. In one aspect, provided herein is an antibody that binds TRGV9, comprising a heavy chain having an amino acid sequence of SEQ ID NO:1801. In one aspect, provided herein is an antibody that binds TRGV9, comprising a light chain having an amino acid sequence of SEQ ID NO:1802. In one aspect, provided herein is an antibody that binds TRGV9, comprising a heavy chain having an amino acid sequence of SEQ ID NO:1801, and a light chain having an amino acid sequence of SEQ ID NO:1802. In one aspect, provided herein is an antibody that binds TRGV9, comprising a VH having an amino acid sequence having at least 95% identity to an amino acid sequence of SEQ ID NO:1799. In one aspect, provided herein is an antibody that binds TRGV9, comprising a VL having an amino acid sequence having at least 95% identity to an amino acid sequence of SEQ ID NO:1800. In one aspect, provided herein is an antibody that binds TRGV9, comprising a VH having an amino acid sequence having at least 95% identity to an amino acid sequence of SEQ ID NO:1799, and a VL having an amino acid sequence having at least 95% identity to an amino acid sequence of SEQ ID NO:1800. In one aspect, provided herein is an antibody that binds TRGV9, comprising a heavy chain having an amino acid sequence having at least 95% identity to an amino acid sequence of SEQ ID NO:1801. In one aspect, provided herein is an antibody that binds TRGV9, comprising a light chain having an amino acid sequence having at least 95% identity to an amino acid sequence of SEQ ID NO:1802. In one aspect, provided herein is an antibody that binds TRGV9, comprising a heavy chain having an amino acid sequence having at least 95% identity to an amino acid sequence of SEQ ID NO:1801, and a light chain having an amino acid sequence having at least 95% identity to an amino acid sequence of SEQ ID NO:1802.

In one aspect, provided herein is an antibody that binds TRGV9, comprising: (i) a VH comprising a VH CDR1, a VH CDR2, and a VH CDR3 having an amino acid sequence of SEQ ID NOs:1803, 1804, and 1805, respectively, and (ii) a VL comprising a VL CDR1, VL CDR2, and VL CDR3 having an amino acid sequence of SEQ ID NOs:1806, 1807, and 1808, respectively. In one aspect, provided herein is an antibody that binds TRGV9, comprising: (i) a VH comprising a VH CDR1, a VH CDR2, and a VH CDR3 having an amino acid sequence of SEQ ID NOs:1809, 1810, and 1811, respectively, and (ii) a VL comprising a VL CDR1, VL CDR2, and VL CDR3 having an amino acid sequence of SEQ ID NOs:1812, 1813, and 1814, respectively. In one aspect, provided herein is an antibody that binds TRGV9, comprising: (i) a VH comprising a VH CDR1, a VH CDR2, and a VH CDR3 having an amino acid sequence of SEQ ID NOs:1815, 1816, and 1817, respectively, and (ii) a VL comprising a VL CDR1, VL CDR2, and VL CDR3 having an amino acid sequence of SEQ ID NOs:1818, 1819, and 1820, respectively. In one aspect, provided herein is an antibody that binds TRGV9, comprising: (i) a VH comprising a VH CDR1, a VH CDR2, and a VH CDR3 having an amino acid sequence of SEQ ID NOs:1821, 1822, and 1823, respectively, and (ii) a VL comprising a VL CDR1, VL CDR2, and VL CDR3 having an amino acid sequence of SEQ ID NOs:1824, 1825, and 1826, respectively. In one aspect, provided herein is an antibody that binds TRGV9, comprising: (i) a VH comprising a VH CDR1, a VH CDR2, and a VH CDR3 having an amino acid sequence of SEQ ID NOs:1827, 1828, and 1829, respectively, and (ii) a VL comprising a VL CDR1, VL CDR2, and VL CDR3 having an amino acid sequence of SEQ ID NOs:1830, 1831, and 1832, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1833; and (ii) a VL comprising a VL CDR1, a VL CDR2, and a VL CDR3 having an amino acid sequence of a VL CDR1, a VL CDR2, and a VL CDR3, respectively, of SEQ ID NO:1834. In one aspect, provided herein is an antibody that binds TRGV9, comprising a VH having an amino acid sequence of SEQ ID NO:1833. In one aspect, provided herein is an antibody that binds TRGV9, comprising a VL having an amino acid sequence of SEQ ID NO:1834. In one aspect, provided herein is an antibody that binds TRGV9, comprising a VH having an amino acid sequence of SEQ ID NO:1833, and a VL having an amino acid sequence of SEQ ID NO:1834. In one aspect, provided herein is an antibody that binds TRGV9, comprising a heavy chain having an amino acid sequence of SEQ ID NO:1835. In one aspect, provided herein is an antibody that binds TRGV9, comprising a light chain having an amino acid sequence of SEQ ID NO:1836. In one aspect, provided herein is an antibody that binds TRGV9, comprising a heavy chain having an amino acid sequence of SEQ ID NO:1835, and a light chain having an amino acid sequence of SEQ ID NO:1836. In one aspect, provided herein is an antibody that binds TRGV9, comprising a VH having an amino acid sequence having at least 95% identity to an amino acid sequence of SEQ ID NO:1833. In one aspect, provided herein is an antibody that binds TRGV9, comprising a VL having an amino acid sequence having at least 95% identity to an amino acid sequence of SEQ ID NO:1834. In one aspect, provided herein is an antibody that binds TRGV9, comprising a VH having an amino acid sequence having at least 95% identity to an amino acid sequence of SEQ ID NO:1833, and a VL having an amino acid sequence having at least 95% identity to an amino acid sequence of SEQ ID NO:1834. In one aspect, provided herein is an antibody that binds TRGV9, comprising a heavy chain having an amino acid sequence having at least 95% identity to an amino acid sequence of SEQ ID NO:1835. In one aspect, provided herein is an antibody that binds TRGV9, comprising a light chain having an amino acid sequence having at least 95% identity to an amino acid sequence of SEQ ID NO:1836. In one aspect, provided herein is an antibody that binds TRGV9, comprising a heavy chain having an amino acid sequence having at least 95% identity to an amino acid sequence of SEQ ID NO:1835, and a light chain having an amino acid sequence having at least 95% identity to an amino acid sequence of SEQ ID NO:1836.

In one aspect, provided herein is an antibody that binds TRGV9, comprising: (i) a VH comprising a VH CDR1, a VH CDR2, and a VH CDR3 having an amino acid sequence of SEQ ID NOs:1837, 1838, and 1839, respectively, and (ii) a VL comprising a VL CDR1, VL CDR2, and VL CDR3 having an amino acid sequence of SEQ ID NOs:1840, 1841, and 1842, respectively. In one aspect, provided herein is an antibody that binds TRGV9, comprising: (i) a VH comprising a VH CDR1, a VH CDR2, and a VH CDR3 having an amino acid sequence of SEQ ID NOs:1843, 1844, and 1845, respectively, and (ii) a VL comprising a VL CDR1, VL CDR2, and VL CDR3 having an amino acid sequence of SEQ ID NOs:1846, 1847, and 1848, respectively. In one aspect, provided herein is an antibody that binds TRGV9, comprising: (i) a VH comprising a VH CDR1, a VH CDR2, and a VH CDR3 having an amino acid sequence of SEQ ID NOs:1849, 1850, and 1851, respectively, and (ii) a VL comprising a VL CDR1, VL CDR2, and VL CDR3 having an amino acid sequence of SEQ ID NOs:1852, 1853, and 1854, respectively. In one aspect, provided herein is an antibody that binds TRGV9, comprising: (i) a VH comprising a VH CDR1, a VH CDR2, and a VH CDR3 having an amino acid sequence of SEQ ID NOs:1855, 1856, and 1857, respectively, and (ii) a VL comprising a VL CDR1, VL CDR2, and VL CDR3 having an amino acid sequence of SEQ ID NOs:1858, 1859, and 1860, respectively. In one aspect, provided herein is an antibody that binds TRGV9, comprising: (i) a VH comprising a VH CDR1, a VH CDR2, and a VH CDR3 having an amino acid sequence of SEQ ID NOs:1861, 1862, and 1863, respectively, and (ii) a VL comprising a VL CDR1, VL CDR2, and VL CDR3 having an amino acid sequence of SEQ ID NOs:1864, 1865, and 1866, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1867; and (ii) a VL comprising a VL CDR1, a VL CDR2, and a VL CDR3 having an amino acid sequence of a VL CDR1, a VL CDR2, and a VL CDR3, respectively, of SEQ ID NO:1868. In one aspect, provided herein is an antibody that binds TRGV9, comprising a VH having an amino acid sequence of SEQ ID NO:1867. In one aspect, provided herein is an antibody that binds TRGV9, comprising a VL having an amino acid sequence of SEQ ID NO:1868. In one aspect, provided herein is an antibody that binds TRGV9, comprising a VH having an amino acid sequence of SEQ ID NO:1867, and a VL having an amino acid sequence of SEQ ID NO:1868. In one aspect, provided herein is an antibody that binds TRGV9, comprising a heavy chain having an amino acid sequence of SEQ ID NO:1869. In one aspect, provided herein is an antibody that binds TRGV9, comprising a light chain having an amino acid sequence of SEQ ID NO:1870. In one aspect, provided herein is an antibody that binds TRGV9, comprising a heavy chain having an amino acid sequence of SEQ ID NO:1869, and a light chain having an amino acid sequence of SEQ ID NO:1870. In one aspect, provided herein is an antibody that binds TRGV9, comprising a VH having an amino acid sequence having at least 95% identity to an amino acid sequence of SEQ ID NO:1867. In one aspect, provided herein is an antibody that binds TRGV9, comprising a VL having an amino acid sequence having at least 95% identity to an amino acid sequence of SEQ ID NO:1868. In one aspect, provided herein is an antibody that binds TRGV9, comprising a VH having an amino acid sequence having at least 95% identity to an amino acid sequence of SEQ ID NO:1867, and a VL having an amino acid sequence having at least 95% identity to an amino acid sequence of SEQ ID NO:1868. In one aspect, provided herein is an antibody that binds TRGV9, comprising a heavy chain having an amino acid sequence having at least 95% identity to an amino acid sequence of SEQ ID NO:1869. In one aspect, provided herein is an antibody that binds TRGV9, comprising a light chain having an amino acid sequence having at least 95% identity to an amino acid sequence of SEQ ID NO:1870. In one aspect, provided herein is an antibody that binds TRGV9, comprising a heavy chain having an amino acid sequence having at least 95% identity to an amino acid sequence of SEQ ID NO:1869, and a light chain having an amino acid sequence having at least 95% identity to an amino acid sequence of SEQ ID NO:1870.

In one aspect, provided herein is an antibody that binds TRGV9, comprising: (i) a VH comprising a VH CDR1, a VH CDR2, and a VH CDR3 having an amino acid sequence of SEQ ID NOs:1871, 1872, and 1873, respectively, and (ii) a VL comprising a VL CDR1, VL CDR2, and VL CDR3 having an amino acid sequence of SEQ ID NOs:1874, 1875, and 1876, respectively. In one aspect, provided herein is an antibody that binds TRGV9, comprising: (i) a VH comprising a VH CDR1, a VH CDR2, and a VH CDR3 having an amino acid sequence of SEQ ID NOs:1877, 1878, and 1879, respectively, and (ii) a VL comprising a VL CDR1, VL CDR2, and VL CDR3 having an amino acid sequence of SEQ ID NOs:1880, 1881, and 1882, respectively. In one aspect, provided herein is an antibody that binds TRGV9, comprising: (i) a VH comprising a VH CDR1, a VH CDR2, and a VH CDR3 having an amino acid sequence of SEQ ID NOs:1883, 1884, and 1885, respectively, and (ii) a VL comprising a VL CDR1, VL CDR2, and VL CDR3 having an amino acid sequence of SEQ ID NOs:1886, 1887, and 1888, respectively. In one aspect, provided herein is an antibody that binds TRGV9, comprising: (i) a VH comprising a VH CDR1, a VH CDR2, and a VH CDR3 having an amino acid sequence of SEQ ID NOs:1889, 1890, and 1891, respectively, and (ii) a VL comprising a VL CDR1, VL CDR2, and VL CDR3 having an amino acid sequence of SEQ ID NOs:1892, 1893, and 1894, respectively. In one aspect, provided herein is an antibody that binds TRGV9, comprising: (i) a VH comprising a VH CDR1, a VH CDR2, and a VH CDR3 having an amino acid sequence of SEQ ID NOs:1895, 1896, and 1897, respectively, and (ii) a VL comprising a VL CDR1, VL CDR2, and VL CDR3 having an amino acid sequence of SEQ ID NOs:1898, 1899, and 1900, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1901; and (ii) a VL comprising a VL CDR1, a VL CDR2, and a VL CDR3 having an amino acid sequence of a VL CDR1, a VL CDR2, and a VL CDR3, respectively, of SEQ ID NO:1902. In one aspect, provided herein is an antibody that binds TRGV9, comprising a VH having an amino acid sequence of SEQ ID NO:1901. In one aspect, provided herein is an antibody that binds TRGV9, comprising a VL having an amino acid sequence of SEQ ID NO:1902. In one aspect, provided herein is an antibody that binds TRGV9, comprising a VH having an amino acid sequence of SEQ ID NO:1901, and a VL having an amino acid sequence of SEQ ID NO:1902. In one aspect, provided herein is an antibody that binds TRGV9, comprising a heavy chain having an amino acid sequence of SEQ ID NO:1903. In one aspect, provided herein is an antibody that binds TRGV9, comprising a light chain having an amino acid sequence of SEQ ID NO:1904. In one aspect, provided herein is an antibody that binds TRGV9, comprising a heavy chain having an amino acid sequence of SEQ ID NO:1903, and a light chain having an amino acid sequence of SEQ ID NO:1904. In one aspect, provided herein is an antibody that binds TRGV9, comprising a VH having an amino acid sequence having at least 95% identity to an amino acid sequence of SEQ ID NO:1901. In one aspect, provided herein is an antibody that binds TRGV9, comprising a VL having an amino acid sequence having at least 95% identity to an amino acid sequence of SEQ ID NO:1902. In one aspect, provided herein is an antibody that binds TRGV9, comprising a VH having an amino acid sequence having at least 95% identity to an amino acid sequence of SEQ ID NO:1901, and a VL having an amino acid sequence having at least 95% identity to an amino acid sequence of SEQ ID NO:1902. In one aspect, provided herein is an antibody that binds TRGV9, comprising a heavy chain having an amino acid sequence having at least 95% identity to an amino acid sequence of SEQ ID NO:1903. In one aspect, provided herein is an antibody that binds TRGV9, comprising a light chain having an amino acid sequence having at least 95% identity to an amino acid sequence of SEQ ID NO:1904. In one aspect, provided herein is an antibody that binds TRGV9, comprising a heavy chain having an amino acid sequence having at least 95% identity to an amino acid sequence of SEQ ID NO:1903, and a light chain having an amino acid sequence having at least 95% identity to an amino acid sequence of SEQ ID NO:1904.

In one aspect, provided herein is an antibody that binds TRGV9, comprising: (i) a VH comprising a VH CDR1, a VH CDR2, and a VH CDR3 having an amino acid sequence of SEQ ID NOs:1905, 1906, and 1907, respectively, and (ii) a VL comprising a VL CDR1, VL CDR2, and VL CDR3 having an amino acid sequence of SEQ ID NOs:1908, 1909, and 1910, respectively. In one aspect, provided herein is an antibody that binds TRGV9, comprising: (i) a VH comprising a VH CDR1, a VH CDR2, and a VH CDR3 having an amino acid sequence of SEQ ID NOs:1911, 1912, and 1913, respectively, and (ii) a VL comprising a VL CDR1, VL CDR2, and VL CDR3 having an amino acid sequence of SEQ ID NOs:1914, 1915, and 1916, respectively. In one aspect, provided herein is an antibody that binds TRGV9, comprising: (i) a VH comprising a VH CDR1, a VH CDR2, and a VH CDR3 having an amino acid sequence of SEQ ID NOs:1917, 1918, and 1919, respectively, and (ii) a VL comprising a VL CDR1, VL CDR2, and VL CDR3 having an amino acid sequence of SEQ ID NOs:1920, 1921, and 1922, respectively. In one aspect, provided herein is an antibody that binds TRGV9, comprising: (i) a VH comprising a VH CDR1, a VH CDR2, and a VH CDR3 having an amino acid sequence of SEQ ID NOs:1923, 1924, and 1925, respectively, and (ii) a VL comprising a VL CDR1, VL CDR2, and VL CDR3 having an amino acid sequence of SEQ ID NOs:1926, 1927, and 1928, respectively. In one aspect, provided herein is an antibody that binds TRGV9, comprising: (i) a VH comprising a VH CDR1, a VH CDR2, and a VH CDR3 having an amino acid sequence of SEQ ID NOs:1929, 1930, and 1931, respectively, and (ii) a VL comprising a VL CDR1, VL CDR2, and VL CDR3 having an amino acid sequence of SEQ ID NOs:1932, 1933, and 1934, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1935; and (ii) a VL comprising a VL CDR1, a VL CDR2, and a VL CDR3 having an amino acid sequence of a VL CDR1, a VL CDR2, and a VL CDR3, respectively, of SEQ ID NO:1936. In one aspect, provided herein is an antibody that binds TRGV9, comprising a VH having an amino acid sequence of SEQ ID NO:1935. In one aspect, provided herein is an antibody that binds TRGV9, comprising a VL having an amino acid sequence of SEQ ID NO:1936. In one aspect, provided herein is an antibody that binds TRGV9, comprising a VH having an amino acid sequence of SEQ ID NO:1935, and a VL having an amino acid sequence of SEQ ID NO:1936. In one aspect, provided herein is an antibody that binds TRGV9, comprising a heavy chain having an amino acid sequence of SEQ ID NO:1937. In one aspect, provided herein is an antibody that binds TRGV9, comprising a light chain having an amino acid sequence of SEQ ID NO:1938. In one aspect, provided herein is an antibody that binds TRGV9, comprising a heavy chain having an amino acid sequence of SEQ ID NO:1937, and a light chain having an amino acid sequence of SEQ ID NO:1938. In one aspect, provided herein is an antibody that binds TRGV9, comprising a VH having an amino acid sequence having at least 95% identity to an amino acid sequence of SEQ ID NO:1935. In one aspect, provided herein is an antibody that binds TRGV9, comprising a VL having an amino acid sequence having at least 95% identity to an amino acid sequence of SEQ ID NO:1936. In one aspect, provided herein is an antibody that binds TRGV9, comprising a VH having an amino acid sequence having at least 95% identity to an amino acid sequence of SEQ ID NO:1935, and a VL having an amino acid sequence having at least 95% identity to an amino acid sequence of SEQ ID NO:1936. In one aspect, provided herein is an antibody that binds TRGV9, comprising a heavy chain having an amino acid sequence having at least 95% identity to an amino acid sequence of SEQ ID NO:1937. In one aspect, provided herein is an antibody that binds TRGV9, comprising a light chain having an amino acid sequence having at least 95% identity to an amino acid sequence of SEQ ID NO:1938. In one aspect, provided herein is an antibody that binds TRGV9, comprising a heavy chain having an amino acid sequence having at least 95% identity to an amino acid sequence of SEQ ID NO:1937, and a light chain having an amino acid sequence having at least 95% identity to an amino acid sequence of SEQ ID NO:1938.

In one aspect, provided herein is an antibody that binds TRGV9, comprising: (i) a VH comprising a VH CDR1, a VH CDR2, and a VH CDR3 having an amino acid sequence of SEQ ID NOs:1939, 1940, and 1941, respectively, and (ii) a VL comprising a VL CDR1, VL CDR2, and VL CDR3 having an amino acid sequence of SEQ ID NOs:1942, 1943, and 1944, respectively. In one aspect, provided herein is an antibody that binds TRGV9, comprising: (i) a VH comprising a VH CDR1, a VH CDR2, and a VH CDR3 having an amino acid sequence of SEQ ID NOs:1945, 1946, and 1947, respectively, and (ii) a VL comprising a VL CDR1, VL CDR2, and VL CDR3 having an amino acid sequence of SEQ ID NOs:1948, 1949, and 1950, respectively. In one aspect, provided herein is an antibody that binds TRGV9, comprising: (i) a VH comprising a VH CDR1, a VH CDR2, and a VH CDR3 having an amino acid sequence of SEQ ID NOs:1951, 1952, and 1953, respectively, and (ii) a VL comprising a VL CDR1, VL CDR2, and VL CDR3 having an amino acid sequence of SEQ ID NOs:1954, 1955, and 1956, respectively. In one aspect, provided herein is an antibody that binds TRGV9, comprising: (i) a VH comprising a VH CDR1, a VH CDR2, and a VH CDR3 having an amino acid sequence of SEQ ID NOs:1957, 1958, and 1959, respectively, and (ii) a VL comprising a VL CDR1, VL CDR2, and VL CDR3 having an amino acid sequence of SEQ ID NOs:1960, 1961, and 1962, respectively. In one aspect, provided herein is an antibody that binds TRGV9, comprising: (i) a VH comprising a VH CDR1, a VH CDR2, and a VH CDR3 having an amino acid sequence of SEQ ID NOs:1963, 1964, and 1965, respectively, and (ii) a VL comprising a VL CDR1, VL CDR2, and VL CDR3 having an amino acid sequence of SEQ ID NOs:1966, 1967, and 1968, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1969; and (ii) a VL comprising a VL CDR1, a VL CDR2, and a VL CDR3 having an amino acid sequence of a VL CDR1, a VL CDR2, and a VL CDR3, respectively, of SEQ ID NO:1970. In one aspect, provided herein is an antibody that binds TRGV9, comprising a VH having an amino acid sequence of SEQ ID NO:1969. In one aspect, provided herein is an antibody that binds TRGV9, comprising a VL having an amino acid sequence of SEQ ID NO:1970. In one aspect, provided herein is an antibody that binds TRGV9, comprising a VH having an amino acid sequence of SEQ ID NO:1969, and a VL having an amino acid sequence of SEQ ID NO:1970. In one aspect, provided herein is an antibody that binds TRGV9, comprising a heavy chain having an amino acid sequence of SEQ ID NO:1971. In one aspect, provided herein is an antibody that binds TRGV9, comprising a light chain having an amino acid sequence of SEQ ID NO:1972. In one aspect, provided herein is an antibody that binds TRGV9, comprising a heavy chain having an amino acid sequence of SEQ ID NO:1971, and a light chain having an amino acid sequence of SEQ ID NO:1972. In one aspect, provided herein is an antibody that binds TRGV9, comprising a VH having an amino acid sequence having at least 95% identity to an amino acid sequence of SEQ ID NO:1969. In one aspect, provided herein is an antibody that binds TRGV9, comprising a VL having an amino acid sequence having at least 95% identity to an amino acid sequence of SEQ ID NO:1970. In one aspect, provided herein is an antibody that binds TRGV9, comprising a VH having an amino acid sequence having at least 95% identity to an amino acid sequence of SEQ ID NO:1969, and a VL having an amino acid sequence having at least 95% identity to an amino acid sequence of SEQ ID NO:1970. In one aspect, provided herein is an antibody that binds TRGV9, comprising a heavy chain having an amino acid sequence having at least 95% identity to an amino acid sequence of SEQ ID NO:1971. In one aspect, provided herein is an antibody that binds TRGV9, comprising a light chain having an amino acid sequence having at least 95% identity to an amino acid sequence of SEQ ID NO:1972. In one aspect, provided herein is an antibody that binds TRGV9, comprising a heavy chain having an amino acid sequence having at least 95% identity to an amino acid sequence of SEQ ID NO:1971, and a light chain having an amino acid sequence having at least 95% identity to an amino acid sequence of SEQ ID NO:1972.

In one aspect, provided herein is an antibody that binds TRGV9, comprising: (i) a VH comprising a VH CDR1, a VH CDR2, and a VH CDR3 having an amino acid sequence of SEQ ID NOs:1973, 1974, and 1975, respectively, and (ii) a VL comprising a VL CDR1, VL CDR2, and VL CDR3 having an amino acid sequence of SEQ ID NOs:1976, 1977, and 1978, respectively. In one aspect, provided herein is an antibody that binds TRGV9, comprising: (i) a VH comprising a VH CDR1, a VH CDR2, and a VH CDR3 having an amino acid sequence of SEQ ID NOs:1979, 1980, and 1981, respectively, and (ii) a VL comprising a VL CDR1, VL CDR2, and VL CDR3 having an amino acid sequence of SEQ ID NOs:1982, 1983, and 1984, respectively. In one aspect, provided herein is an antibody that binds TRGV9, comprising: (i) a VH comprising a VH CDR1, a VH CDR2, and a VH CDR3 having an amino acid sequence of SEQ ID NOs:1985, 1986, and 1987, respectively, and (ii) a VL comprising a VL CDR1, VL CDR2, and VL CDR3 having an amino acid sequence of SEQ ID NOs:1988, 1989, and 1990, respectively. In one aspect, provided herein is an antibody that binds TRGV9, comprising: (i) a VH comprising a VH CDR1, a VH CDR2, and a VH CDR3 having an amino acid sequence of SEQ ID NOs:1991, 1992, and 1993, respectively, and (ii) a VL comprising a VL CDR1, VL CDR2, and VL CDR3 having an amino acid sequence of SEQ ID NOs:1994, 1995, and 1996, respectively. In one aspect, provided herein is an antibody that binds TRGV9, comprising: (i) a VH comprising a VH CDR1, a VH CDR2, and a VH CDR3 having an amino acid sequence of SEQ ID NOs:1997, 1998, and 1999, respectively, and (ii) a VL comprising a VL CDR1, VL CDR2, and VL CDR3 having an amino acid sequence of SEQ ID NOs:2000, 2001, and 2002, respectively. In one aspect, provided herein is an antibody that binds TRGV9, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2003; and (ii) a VL comprising a VL CDR1, a VL CDR2, and a VL CDR3 having an amino acid sequence of a VL CDR1, a VL CDR2, and a VL CDR3, respectively, of SEQ ID NO:2004. In one aspect, provided herein is an antibody that binds TRGV9, comprising a VH having an amino acid sequence of SEQ ID NO:2003. In one aspect, provided herein is an antibody that binds TRGV9, comprising a VL having an amino acid sequence of SEQ ID NO:2004. In one aspect, provided herein is an antibody that binds TRGV9, comprising a VH having an amino acid sequence of SEQ ID NO:2003, and a VL having an amino acid sequence of SEQ ID NO:2004. In one aspect, provided herein is an antibody that binds TRGV9, comprising a heavy chain having an amino acid sequence of SEQ ID NO:2005. In one aspect, provided herein is an antibody that binds TRGV9, comprising a light chain having an amino acid sequence of SEQ ID NO:2006. In one aspect, provided herein is an antibody that binds TRGV9, comprising a heavy chain having an amino acid sequence of SEQ ID NO:2005, and a light chain having an amino acid sequence of SEQ ID NO:2006. In one aspect, provided herein is an antibody that binds TRGV9, comprising a VH having an amino acid sequence having at least 95% identity to an amino acid sequence of SEQ ID NO:2003. In one aspect, provided herein is an antibody that binds TRGV9, comprising a VL having an amino acid sequence having at least 95% identity to an amino acid sequence of SEQ ID NO:2004. In one aspect, provided herein is an antibody that binds TRGV9, comprising a VH having an amino acid sequence having at least 95% identity to an amino acid sequence of SEQ ID NO:2003, and a VL having an amino acid sequence having at least 95% identity to an amino acid sequence of SEQ ID NO:2004. In one aspect, provided herein is an antibody that binds TRGV9, comprising a heavy chain having an amino acid sequence having at least 95% identity to an amino acid sequence of SEQ ID NO:2005. In one aspect, provided herein is an antibody that binds TRGV9, comprising a light chain having an amino acid sequence having at least 95% identity to an amino acid sequence of SEQ ID NO:2006. In one aspect, provided herein is an antibody that binds TRGV9, comprising a heavy chain having an amino acid sequence having at least 95% identity to an amino acid sequence of SEQ ID NO:2005, and a light chain having an amino acid sequence having at least 95% identity to an amino acid sequence of SEQ ID NO:2006.

In one aspect, provided herein is an antibody that binds TRGV9. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:168. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:168. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:201. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:202. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:201; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:202. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:269. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:269; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:304. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:304. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:337. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:338. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:337; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:338. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:440. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:440. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:609. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:610. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:609; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:610. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:644. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:644. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:712. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:712. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:847. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:848. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:847; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:848. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:881. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:882. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:881; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:882. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:915. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:916. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:915; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:916. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:949. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:950. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:949; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:950. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:983. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:984. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:983; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:984. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1017. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1018. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1017; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1018. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1051. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1052. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1051; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1052. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1085. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1086. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1085; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1086. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1119. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1120. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1119; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1120. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1153. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1154. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1153; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1154. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1187. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1188. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1187; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1188. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1221. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1222. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1221; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1222. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1255. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1256. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1255; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1256. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1289. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1290. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1289; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1290. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1323. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1324. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1323; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1324. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1357. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1358. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1357; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1358. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1391. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1392. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1391; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1392. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1425. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1426. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1425; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1426. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1459. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1460. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1459; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1460. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1493. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1494. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1493; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1494. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1527. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1528. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1527; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1528. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1561. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1562. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1561; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1562. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1595. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1596. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1595; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1596. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1629. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1630. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1629; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1630. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1663. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1664. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1663; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1664. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1697. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1698. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1697; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1698. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1731. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1732. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1731; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1732. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1765. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1766. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1765; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1766. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1799. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1800. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1799; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1800. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1833. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1834. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1833; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1834. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1867. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1868. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1867; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1868. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1901. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1902. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1901; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1902. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1935. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1936. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1935; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1936. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1969. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1970. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1969; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1970. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2003. In one embodiment, the TRGV9 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2004. In one embodiment, the TRGV9 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2003; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2004. In some embodiments, the TRGV9 antibody is a multispecific antibody. In some embodiments, the TRGV9 antibody is a bispecific antibody. In some embodiments, the TRGV9 antibody is a trispecific antibody. In some embodiments, the TRGV9 antibody is a quadraspecific antibody.

In one aspect, provided herein is an antibody that binds TRGV9, comprising a VH CDR1 having an amino acid sequence of SEQ ID NO:25, wherein the third amino acid is A or T; a VH CDR2 having an amino acid sequence of SEQ ID NO:26, wherein the second amino acid is G or S, the fourth amino acid is S or T, the sixth amino acid is D or S; and a VH CDR3 having an amino acid sequence of SEQ ID NO:27, wherein the first amino acid is A, G, or V, the second amino acid is K, R, or T, the fourth amino acid is D, H, or G, the fifth amino acid is D, E, or V, the sixth amino acid is F, L, or Y, the eighth amino acid is A, G, P, or V, the ninth amino acid is F or L, the third amino acid is I of V. In one aspect, provided herein is an antibody that binds TRGV9, comprising a VL CDR1 having an amino acid sequence of SEQ ID NO:28, wherein the third amino acid is I or V, the fourth amino acid is R or S; a VL CDR2 having an amino acid sequence of SEQ ID NO:29, wherein the first amino acid is A, P or S; and a VL CDR3 having an amino acid sequence of SEQ ID NO:30, wherein the third amino acid is F or Y. In one aspect, provided herein is an antibody that binds TRGV9, comprising a VH CDR1 having an amino acid sequence of SEQ ID NO:25, wherein the third amino acid is A or T; a VH CDR2 having an amino acid sequence of SEQ ID NO:26, wherein the second amino acid is G or S, the fourth amino acid is S or T, the sixth amino acid is D or S; and a VH CDR3 having an amino acid sequence of SEQ ID NO:27, wherein the first amino acid is A, G, or V, the second amino acid is K, R, or T, the fourth amino acid is D, H, or G, the fifth amino acid is D, E, or V, the sixth amino acid is F, L, or Y, the eighth amino acid is A, G, P, or V, the ninth amino acid is F or L, the third amino acid is I of V; a VL CDR1 having an amino acid sequence of SEQ ID NO:28, wherein the third amino acid is I or V, the fourth amino acid is R or S; a VL CDR2 having an amino acid sequence of SEQ ID NO:29, wherein the first amino acid is A, P or S; and a VL CDR3 having an amino acid sequence of SEQ ID NO:30, wherein the third amino acid is F or Y.

In one aspect, provided herein is an antibody that binds TRGV9, comprising a VH CDR1 having an amino acid sequence of SEQ ID NO:93; a VH CDR2 having an amino acid sequence of SEQ ID NO:94; a VH CDR3 having an amino acid sequence of SEQ ID NO:95. In one aspect, provided herein is an antibody that binds TRGV9, comprising a VL CDR1 having an amino acid sequence of SEQ ID NO:96; a VL CDR2 having an amino acid sequence of SEQ ID NO:97; and a VL CDR3 having an amino acid sequence of SEQ ID NO:98, wherein the third amino acid is A or Y, the fourth is N or S, the sixth is F or W, and the eighth is R or W. In one aspect, provided herein is an antibody that binds TRGV9, comprising a VH CDR1 having an amino acid sequence of SEQ ID NO:93; a VH CDR2 having an amino acid sequence of SEQ ID NO:94; a VH CDR3 having an amino acid sequence of SEQ ID NO:95; a VL CDR1 having an amino acid sequence of SEQ ID NO:96; a VL CDR2 having an amino acid sequence of SEQ ID NO:97; and a VL CDR3 having an amino acid sequence of SEQ ID NO:98, wherein the third amino acid is A or Y, the fourth is N or S, the sixth is F or W, and the eighth is R or W.

In one aspect, provided herein is an antibody that binds TRGV9, comprising a VH CDR1 having an amino acid sequence of SEQ ID NO:127, wherein the first amino acid if G or R, the fourth amino acid is F or L, the fifth amino acid if R or S; a VH CDR2 having an amino acid sequence of SEQ ID NO:128, wherein the second amino acid is S or T, the third amino acid is S or T, the fourth amino acid is S or T, the sixth amino acid is G, S, or Y; a VH CDR3 having an amino acid sequence of SEQ ID NO:129, wherein the fourth amino acid is L or R, the sixth amino acid if I or V, the seventh amino acid A, G, or R, the eighth amino acid is G or B, the ninth amino acid is A, T, or V, the tenth amino acid is D or G, the eleventh amino acid is D or Y, the sixteenth amino acid is L or M. In one aspect, provided herein is an antibody that binds TRGV9, comprising a VL CDR1 having an amino acid sequence of SEQ ID NO:130; a VL CDR2 having an amino acid sequence of SEQ ID NO:131; and a VL CDR3 having an amino acid sequence of SEQ ID NO:132. In one aspect, provided herein is an antibody that binds TRGV9, comprising a VH CDR1 having an amino acid sequence of SEQ ID NO:127, wherein the first amino acid if G or R, the fourth amino acid is F or L, the fifth amino acid if R or S; a VH CDR2 having an amino acid sequence of SEQ ID NO:128, wherein the second amino acid is S or T, the third amino acid is S or T, the fourth amino acid is S or T, the sixth amino acid is G, S, or Y; a VH CDR3 having an amino acid sequence of SEQ ID NO:129, wherein the fourth amino acid is L or R, the sixth amino acid if I or V, the seventh amino acid A, G, or R, the eighth amino acid is G or B, the ninth amino acid is A, T, or V, the tenth amino acid is D or G, the eleventh amino acid is D or Y, the sixteenth amino acid is L or M; a VL CDR1 having an amino acid sequence of SEQ ID NO:130; a VL CDR2 having an amino acid sequence of SEQ ID NO:131; and a VL CDR3 having an amino acid sequence of SEQ ID NO:132.

In one aspect, provided herein is an antibody that binds TRGV9, comprising a VH CDR1 having an amino acid sequence of SEQ ID NO:297, wherein the fifth amino acid is N or S, the sixth amino acid is N or S; a VH CDR2 having an amino acid sequence of SEQ ID NO:298; a VH CDR3 having an amino acid sequence of SEQ ID NO:299, wherein the fourth amino acid is R or V, the sixth amino acid is I or V, the seventh amino acid is G or T, the eighth amino acid is G or T, the twelfth amino acid is S or Y, the fourteenth amino acid is A or G. In one aspect, provided herein is an antibody that binds TRGV9, comprising a VL CDR1 having an amino acid sequence of SEQ ID NO:300, wherein the fourth amino acid is A or S; a VL CDR2 having an amino acid sequence of SEQ ID NO:301 and a VL CDR3 having an amino acid sequence of SEQ ID NO:302. In one aspect, provided herein is an antibody that binds TRGV9, comprising a VH CDR1 having an amino acid sequence of SEQ ID NO:297, wherein the fifth amino acid is N or S, the sixth amino acid is N or S; a VH CDR2 having an amino acid sequence of SEQ ID NO:298; a VH CDR3 having an amino acid sequence of SEQ ID NO:299, wherein the fourth amino acid is R or V, the sixth amino acid is I or V, the seventh amino acid is G or T, the eighth amino acid is G or T, the twelfth amino acid is S or Y, the fourteenth amino acid is A or G; a VL CDR1 having an amino acid sequence of SEQ ID NO:300, wherein the fourth amino acid is A or S; a VL CDR2 having an amino acid sequence of SEQ ID NO:301 and a VL CDR3 having an amino acid sequence of SEQ ID NO:302.

In one aspect, provided herein is an antibody that binds TRGV9, comprising a VH CDR1 having an amino acid sequence of SEQ ID NO:569; a VH CDR2 having an amino acid sequence of SEQ ID NO:570; a VH CDR3 having an amino acid sequence of SEQ ID NO:571. In one aspect, provided herein is an antibody that binds TRGV9, comprising a VL CDR1 having an amino acid sequence of SEQ ID NO:572, wherein the second amino acid is G or N, the third amino acid is I or V, the fourth amino acid is A or S, the fifth amino acid is S or there is no amino acid added to the fifth position and sequence continues to the sixth position; a VL CDR2 having an amino acid sequence of SEQ ID NO:573, wherein the first amino acid is A or G, the third amino acid is C or S; and a VL CDR3 having an amino acid sequence of SEQ ID NO:574, wherein the second amino acid is K or Q, the fourth amino acid is G or N, the sixth amino acid is A or S, the ninth amino acid is Y or W. In one aspect, provided herein is an antibody that binds TRGV9, comprising a VH CDR1 having an amino acid sequence of SEQ ID NO:569; a VH CDR2 having an amino acid sequence of SEQ ID NO:570; a VH CDR3 having an amino acid sequence of SEQ ID NO:571; a VL CDR1 having an amino acid sequence of SEQ ID NO:572, wherein the second amino acid is G or N, the third amino acid is I or V, the fourth amino acid is A or S, the fifth amino acid is S or there is no amino acid added to the fifth position and sequence continues to the sixth position; a VL CDR2 having an amino acid sequence of SEQ ID NO:573, wherein the first amino acid is A or G, the third amino acid is C or S; and a VL CDR3 having an amino acid sequence of SEQ ID NO:574, wherein the second amino acid is K or Q, the fourth amino acid is G or N, the sixth amino acid is A or S, the ninth amino acid is Y or W.

In one aspect, provided herein is an antibody that binds TRGV9, comprising a VH CDR1 having an amino acid sequence of SEQ ID NO:739; a VH CDR2 having an amino acid sequence of SEQ ID NO:740; a VH CDR3 having an amino acid sequence of SEQ ID NO:741, wherein the third amino acid is D or E, the fourth amino acid is I or L, the sixth amino acid I or V, the eleventh amino acid is M or Y, the twelfth amino acid is D or Y, the thirteenth amino acid is Q or Y, the fourteenth amino acid is D or Y. In one aspect, provided herein is an antibody that binds TRGV9, comprising a VL CDR1 having an amino acid sequence of SEQ ID NO:742, wherein the third amino acid is F or V, the sixth amino acid is N or S; a VL CDR2 having an amino acid sequence of SEQ ID NO:743; and a VL CDR3 having an amino acid sequence of SEQ ID NO:744. In one aspect, provided herein is an antibody that binds TRGV9, comprising a VH CDR1 having an amino acid sequence of SEQ ID NO:739; a VH CDR2 having an amino acid sequence of SEQ ID NO:740; a VH CDR3 having an amino acid sequence of SEQ ID NO:741, wherein the third amino acid is D or E, the fourth amino acid is I or L, the sixth amino acid I or V, the eleventh amino acid is M or Y, the twelfth amino acid is D or Y, the thirteenth amino acid is Q or Y, the fourteenth amino acid is D or Y; a VL CDR1 having an amino acid sequence of SEQ ID NO:742, wherein the third amino acid is F or V, the sixth amino acid is N or S; a VL CDR2 having an amino acid sequence of SEQ ID NO:743; and a VL CDR3 having an amino acid sequence of SEQ ID NO:744.

In one aspect, provided herein is an antibody that binds TRGV9, comprising a VH CDR1 having an amino acid sequence of SEQ ID NO:1181, wherein the fifth amino acid is G or S, the sixth amino acid is A or S; a VH CDR2 having an amino acid sequence of SEQ ID NO:1182, wherein the third amino acid is S or T; a VH CDR3 having an amino acid sequence of SEQ ID NO:1183, wherein the sixth amino acid is I or M, the seventh amino acid is L or V, the eighth amino acid is A or S, the ninth amino acid is P or T, the eleventh amino acid is K or R. In one aspect, provided herein is an antibody that binds TRGV9, comprising a VL CDR1 having an amino acid sequence of SEQ ID NO:1184, wherein the fifth amino acid is D or N, the eighth amino acid is A or D; a VL CDR2 having an amino acid sequence of SEQ ID NO:1185; and a VL CDR3 having an amino acid sequence of SEQ ID NO:1186. In one aspect, provided herein is an antibody that binds TRGV9, comprising a VH CDR1 having an amino acid sequence of SEQ ID NO:1181, wherein the fifth amino acid is G or S, the sixth amino acid is A or S; a VH CDR2 having an amino acid sequence of SEQ ID NO:1182, wherein the third amino acid is S or T; a VH CDR3 having an amino acid sequence of SEQ ID NO:1183, wherein the sixth amino acid is I or M, the seventh amino acid is L or V, the eighth amino acid is A or S, the ninth amino acid is P or T, the eleventh amino acid is K or R; a VL CDR1 having an amino acid sequence of SEQ ID NO:1184, wherein the fifth amino acid is D or N, the eighth amino acid is A or D; a VL CDR2 having an amino acid sequence of SEQ ID NO:1185; and a VL CDR3 having an amino acid sequence of SEQ ID NO:1186.

In one aspect, provided herein is an antibody that binds to TRDV2. In some embodiments, the antibody comprises a heavy chain variable region and a light chain variable region. In some embodiments, the TRDV2 antibody is a humanized antibody.

In one aspect, provided herein is an antibody that binds TRDV2, comprising: (i) a VH comprising a VH CDR1, a VH CDR2, and a VH CDR3 having an amino acid sequence of SEQ ID NOs:2007, 2008, and 2009, respectively, and (ii) a VL comprising a VL CDR1, VL CDR2, and VL CDR3 having an amino acid sequence of SEQ ID NOs:2010, 2011, and 2012, respectively. In one aspect, provided herein is an antibody that binds TRDV2, comprising: (i) a VH comprising a VH CDR1, a VH CDR2, and a VH CDR3 having an amino acid sequence of SEQ ID NOs:2013, 2014, and 2015, respectively, and (ii) a VL comprising a VL CDR1, VL CDR2, and VL CDR3 having an amino acid sequence of SEQ ID NOs:2016, 2017, and 2018, respectively. In one aspect, provided herein is an antibody that binds TRDV2, comprising: (i) a VH comprising a VH CDR1, a VH CDR2, and a VH CDR3 having an amino acid sequence of SEQ ID NOs:2019, 2020, and 2021, respectively, and (ii) a VL comprising a VL CDR1, VL CDR2, and VL CDR3 having an amino acid sequence of SEQ ID NOs:2022, 2023, and 2024, respectively. In one aspect, provided herein is an antibody that binds TRDV2, comprising: (i) a VH comprising a VH CDR1, a VH CDR2, and a VH CDR3 having an amino acid sequence of SEQ ID NOs:2025, 2026, and 2027, respectively, and (ii) a VL comprising a VL CDR1, VL CDR2, and VL CDR3 having an amino acid sequence of SEQ ID NOs:2028, 2029, and 2030, respectively. In one aspect, provided herein is an antibody that binds TRDV2, comprising: (i) a VH comprising a VH CDR1, a VH CDR2, and a VH CDR3 having an amino acid sequence of SEQ ID NOs:2031, 2032, and 2033, respectively, and (ii) a VL comprising a VL CDR1, VL CDR2, and VL CDR3 having an amino acid sequence of SEQ ID NOs:2034, 2035, and 2036, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2037; and (ii) a VL comprising a VL CDR1, a VL CDR2, and a VL CDR3 having an amino acid sequence of a VL CDR1, a VL CDR2, and a VL CDR3, respectively, of SEQ ID NO:2038. In one aspect, provided herein is an antibody that binds TRDV2, comprising a VH having an amino acid sequence of SEQ ID NO:2037. In one aspect, provided herein is an antibody that binds TRDV2, comprising a VL having an amino acid sequence of SEQ ID NO:2038. In one aspect, provided herein is an antibody that binds TRDV2, comprising a VH having an amino acid sequence of SEQ ID NO:2037, and a VL having an amino acid sequence of SEQ ID NO:2038. In one aspect, provided herein is an antibody that binds TRDV2, comprising a heavy chain having an amino acid sequence of SEQ ID NO:2039. In one aspect, provided herein is an antibody that binds TRDV2, comprising a light chain having an amino acid sequence of SEQ ID NO:2040. In one aspect, provided herein is an antibody that binds TRDV2, comprising a heavy chain having an amino acid sequence of SEQ ID NO:2039, and a light chain having an amino acid sequence of SEQ ID NO:2040. In one aspect, provided herein is an antibody that binds TRDV2, comprising a VH having an amino acid sequence having at least 95% identity to an amino acid sequence of SEQ ID NO:2037. In one aspect, provided herein is an antibody that binds TRDV2, comprising a VL having an amino acid sequence having at least 95% identity to an amino acid sequence of SEQ ID NO:2038. In one aspect, provided herein is an antibody that binds TRDV2, comprising a VH having an amino acid sequence having at least 95% identity to an amino acid sequence of SEQ ID NO:2037, and a VL having an amino acid sequence having at least 95% identity to an amino acid sequence of SEQ ID NO:2038. In one aspect, provided herein is an antibody that binds TRDV2, comprising a heavy chain having an amino acid sequence having at least 95% identity to an amino acid sequence of SEQ ID NO:2039. In one aspect, provided herein is an antibody that binds TRDV2, comprising a light chain having an amino acid sequence having at least 95% identity to an amino acid sequence of SEQ ID NO:2040. In one aspect, provided herein is an antibody that binds TRDV2, comprising a heavy chain having an amino acid sequence having at least 95% identity to an amino acid sequence of SEQ ID NO:2039, and a light chain having an amino acid sequence having at least 95% identity to an amino acid sequence of SEQ ID NO:2040.

In one aspect, provided herein is an antibody that binds TRDV2, comprising: (i) a VH comprising a VH CDR1, a VH CDR2, and a VH CDR3 having an amino acid sequence of SEQ ID NOs:2041, 2042, and 2043, respectively, and (ii) a VL comprising a VL CDR1, VL CDR2, and VL CDR3 having an amino acid sequence of SEQ ID NOs:2044, 2045, and 2046, respectively. In one aspect, provided herein is an antibody that binds TRDV2, comprising: (i) a VH comprising a VH CDR1, a VH CDR2, and a VH CDR3 having an amino acid sequence of SEQ ID NOs:2047, 2048, and 2049, respectively, and (ii) a VL comprising a VL CDR1, VL CDR2, and VL CDR3 having an amino acid sequence of SEQ ID NOs:2050, 2051, and 2052, respectively. In one aspect, provided herein is an antibody that binds TRDV2, comprising: (i) a VH comprising a VH CDR1, a VH CDR2, and a VH CDR3 having an amino acid sequence of SEQ ID NOs:2053, 2054, and 2055, respectively, and (ii) a VL comprising a VL CDR1, VL CDR2, and VL CDR3 having an amino acid sequence of SEQ ID NOs:2056, 2057, and 2058, respectively. In one aspect, provided herein is an antibody that binds TRDV2, comprising: (i) a VH comprising a VH CDR1, a VH CDR2, and a VH CDR3 having an amino acid sequence of SEQ ID NOs:2059, 2060, and 2061, respectively, and (ii) a VL comprising a VL CDR1, VL CDR2, and VL CDR3 having an amino acid sequence of SEQ ID NOs:2062, 2063, and 2064, respectively. In one aspect, provided herein is an antibody that binds TRDV2, comprising: (i) a VH comprising a VH CDR1, a VH CDR2, and a VH CDR3 having an amino acid sequence of SEQ ID NOs:2065, 2066, and 2067, respectively, and (ii) a VL comprising a VL CDR1, VL CDR2, and VL CDR3 having an amino acid sequence of SEQ ID NOs:2068, 2069, and 2070, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2071; and (ii) a VL comprising a VL CDR1, a VL CDR2, and a VL CDR3 having an amino acid sequence of a VL CDR1, a VL CDR2, and a VL CDR3, respectively, of SEQ ID NO:2072. In one aspect, provided herein is an antibody that binds TRDV2, comprising a VH having an amino acid sequence of SEQ ID NO:2071. In one aspect, provided herein is an antibody that binds TRDV2, comprising a VL having an amino acid sequence of SEQ ID NO:2072. In one aspect, provided herein is an antibody that binds TRDV2, comprising a VH having an amino acid sequence of SEQ ID NO:2071, and a VL having an amino acid sequence of SEQ ID NO:2072. In one aspect, provided herein is an antibody that binds TRDV2, comprising a heavy chain having an amino acid sequence of SEQ ID NO:2073. In one aspect, provided herein is an antibody that binds TRDV2, comprising a light chain having an amino acid sequence of SEQ ID NO:2074. In one aspect, provided herein is an antibody that binds TRDV2, comprising a heavy chain having an amino acid sequence of SEQ ID NO:2073, and a light chain having an amino acid sequence of SEQ ID NO:2074. In one aspect, provided herein is an antibody that binds TRDV2, comprising a VH having an amino acid sequence having at least 95% identity to an amino acid sequence of SEQ ID NO:2071. In one aspect, provided herein is an antibody that binds TRDV2, comprising a VL having an amino acid sequence having at least 95% identity to an amino acid sequence of SEQ ID NO:2072. In one aspect, provided herein is an antibody that binds TRDV2, comprising a VH having an amino acid sequence having at least 95% identity to an amino acid sequence of SEQ ID NO:2071, and a VL having an amino acid sequence having at least 95% identity to an amino acid sequence of SEQ ID NO:2072. In one aspect, provided herein is an antibody that binds TRDV2, comprising a heavy chain having an amino acid sequence having at least 95% identity to an amino acid sequence of SEQ ID NO:2073. In one aspect, provided herein is an antibody that binds TRDV2, comprising a light chain having an amino acid sequence having at least 95% identity to an amino acid sequence of SEQ ID NO:2074. In one aspect, provided herein is an antibody that binds TRDV2, comprising a heavy chain having an amino acid sequence having at least 95% identity to an amino acid sequence of SEQ ID NO:2073, and a light chain having an amino acid sequence having at least 95% identity to an amino acid sequence of SEQ ID NO:2074.

In one aspect, provided herein is an antibody that binds TRDV2, comprising: (i) a VH comprising a VH CDR1, a VH CDR2, and a VH CDR3 having an amino acid sequence of SEQ ID NOs:2075, 2076, and 2077, respectively, and (ii) a VL comprising a VL CDR1, VL CDR2, and VL CDR3 having an amino acid sequence of SEQ ID NOs:2078, 2079, and 2080, respectively. In one aspect, provided herein is an antibody that binds TRDV2, comprising: (i) a VH comprising a VH CDR1, a VH CDR2, and a VH CDR3 having an amino acid sequence of SEQ ID NOs:2081, 2082, and 2083, respectively, and (ii) a VL comprising a VL CDR1, VL CDR2, and VL CDR3 having an amino acid sequence of SEQ ID NOs:2084, 2085, and 2086, respectively. In one aspect, provided herein is an antibody that binds TRDV2, comprising: (i) a VH comprising a VH CDR1, a VH CDR2, and a VH CDR3 having an amino acid sequence of SEQ ID NOs:2087, 2088, and 2089, respectively, and (ii) a VL comprising a VL CDR1, VL CDR2, and VL CDR3 having an amino acid sequence of SEQ ID NOs:2090, 2091, and 2092, respectively. In one aspect, provided herein is an antibody that binds TRDV2, comprising: (i) a VH comprising a VH CDR1, a VH CDR2, and a VH CDR3 having an amino acid sequence of SEQ ID NOs:2093, 2094, and 2095, respectively, and (ii) a VL comprising a VL CDR1, VL CDR2, and VL CDR3 having an amino acid sequence of SEQ ID NOs:2096, 2097, and 2098, respectively. In one aspect, provided herein is an antibody that binds TRDV2, comprising: (i) a VH comprising a VH CDR1, a VH CDR2, and a VH CDR3 having an amino acid sequence of SEQ ID NOs:2099, 2100, and 2101, respectively, and (ii) a VL comprising a VL CDR1, VL CDR2, and VL CDR3 having an amino acid sequence of SEQ ID NOs:2102, 2103, and 2104, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2105; and (ii) a VL comprising a VL CDR1, a VL CDR2, and a VL CDR3 having an amino acid sequence of a VL CDR1, a VL CDR2, and a VL CDR3, respectively, of SEQ ID NO:2106. In one aspect, provided herein is an antibody that binds TRDV2, comprising a VH having an amino acid sequence of SEQ ID NO:2105. In one aspect, provided herein is an antibody that binds TRDV2, comprising a VL having an amino acid sequence of SEQ ID NO:2106. In one aspect, provided herein is an antibody that binds TRDV2, comprising a VH having an amino acid sequence of SEQ ID NO:2105, and a VL having an amino acid sequence of SEQ ID NO:2106. In one aspect, provided herein is an antibody that binds TRDV2, comprising a heavy chain having an amino acid sequence of SEQ ID NO:2107. In one aspect, provided herein is an antibody that binds TRDV2, comprising a light chain having an amino acid sequence of SEQ ID NO:2108. In one aspect, provided herein is an antibody that binds TRDV2, comprising a heavy chain having an amino acid sequence of SEQ ID NO:2107, and a light chain having an amino acid sequence of SEQ ID NO:2108. In one aspect, provided herein is an antibody that binds TRDV2, comprising a VH having an amino acid sequence having at least 95% identity to an amino acid sequence of SEQ ID NO:2105. In one aspect, provided herein is an antibody that binds TRDV2, comprising a VL having an amino acid sequence having at least 95% identity to an amino acid sequence of SEQ ID NO:2106. In one aspect, provided herein is an antibody that binds TRDV2, comprising a VH having an amino acid sequence having at least 95% identity to an amino acid sequence of SEQ ID NO:2105, and a VL having an amino acid sequence having at least 95% identity to an amino acid sequence of SEQ ID NO:2106. In one aspect, provided herein is an antibody that binds TRDV2, comprising a heavy chain having an amino acid sequence having at least 95% identity to an amino acid sequence of SEQ ID NO:2107. In one aspect, provided herein is an antibody that binds TRDV2, comprising a light chain having an amino acid sequence having at least 95% identity to an amino acid sequence of SEQ ID NO:2108. In one aspect, provided herein is an antibody that binds TRDV2, comprising a heavy chain having an amino acid sequence having at least 95% identity to an amino acid sequence of SEQ ID NO:2107, and a light chain having an amino acid sequence having at least 95% identity to an amino acid sequence of SEQ ID NO:2108.

In one aspect, provided herein is an antibody that binds TRDV2, comprising: (i) a VH comprising a VH CDR1, a VH CDR2, and a VH CDR3 having an amino acid sequence of SEQ ID NOs:2109, 2110, and 2111, respectively, and (ii) a VL comprising a VL CDR1, VL CDR2, and VL CDR3 having an amino acid sequence of SEQ ID NOs:2112, 2113, and 2114, respectively. In one aspect, provided herein is an antibody that binds TRDV2, comprising: (i) a VH comprising a VH CDR1, a VH CDR2, and a VH CDR3 having an amino acid sequence of SEQ ID NOs:2115, 2116, and 2117, respectively, and (ii) a VL comprising a VL CDR1, VL CDR2, and VL CDR3 having an amino acid sequence of SEQ ID NOs:2118, 2119, and 2120, respectively. In one aspect, provided herein is an antibody that binds TRDV2, comprising: (i) a VH comprising a VH CDR1, a VH CDR2, and a VH CDR3 having an amino acid sequence of SEQ ID NOs:2121, 2122, and 2123, respectively, and (ii) a VL comprising a VL CDR1, VL CDR2, and VL CDR3 having an amino acid sequence of SEQ ID NOs:2124, 2125, and 2126, respectively. In one aspect, provided herein is an antibody that binds TRDV2, comprising: (i) a VH comprising a VH CDR1, a VH CDR2, and a VH CDR3 having an amino acid sequence of SEQ ID NOs:2127, 2128, and 2129, respectively, and (ii) a VL comprising a VL CDR1, VL CDR2, and VL CDR3 having an amino acid sequence of SEQ ID NOs:2130, 2131, and 2132, respectively. In one aspect, provided herein is an antibody that binds TRDV2, comprising: (i) a VH comprising a VH CDR1, a VH CDR2, and a VH CDR3 having an amino acid sequence of SEQ ID NOs:2133, 2134, and 2135, respectively, and (ii) a VL comprising a VL CDR1, VL CDR2, and VL CDR3 having an amino acid sequence of SEQ ID NOs:2136, 2137, and 2138, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2139; and (ii) a VL comprising a VL CDR1, a VL CDR2, and a VL CDR3 having an amino acid sequence of a VL CDR1, a VL CDR2, and a VL CDR3, respectively, of SEQ ID NO:2140. In one aspect, provided herein is an antibody that binds TRDV2, comprising a VH having an amino acid sequence of SEQ ID NO:2139. In one aspect, provided herein is an antibody that binds TRDV2, comprising a VL having an amino acid sequence of SEQ ID NO:2140. In one aspect, provided herein is an antibody that binds TRDV2, comprising a VH having an amino acid sequence of SEQ ID NO:2139, and a VL having an amino acid sequence of SEQ ID NO:2140. In one aspect, provided herein is an antibody that binds TRDV2, comprising a heavy chain having an amino acid sequence of SEQ ID NO:2141. In one aspect, provided herein is an antibody that binds TRDV2, comprising a light chain having an amino acid sequence of SEQ ID NO:2142. In one aspect, provided herein is an antibody that binds TRDV2, comprising a heavy chain having an amino acid sequence of SEQ ID NO:2141, and a light chain having an amino acid sequence of SEQ ID NO:2142. In one aspect, provided herein is an antibody that binds TRDV2, comprising a VH having an amino acid sequence having at least 95% identity to an amino acid sequence of SEQ ID NO:2139. In one aspect, provided herein is an antibody that binds TRDV2, comprising a VL having an amino acid sequence having at least 95% identity to an amino acid sequence of SEQ ID NO:2140. In one aspect, provided herein is an antibody that binds TRDV2, comprising a VH having an amino acid sequence having at least 95% identity to an amino acid sequence of SEQ ID NO:2139, and a VL having an amino acid sequence having at least 95% identity to an amino acid sequence of SEQ ID NO:2140. In one aspect, provided herein is an antibody that binds TRDV2, comprising a heavy chain having an amino acid sequence having at least 95% identity to an amino acid sequence of SEQ ID NO:2141. In one aspect, provided herein is an antibody that binds TRDV2, comprising a light chain having an amino acid sequence having at least 95% identity to an amino acid sequence of SEQ ID NO:2142. In one aspect, provided herein is an antibody that binds TRDV2, comprising a heavy chain having an amino acid sequence having at least 95% identity to an amino acid sequence of SEQ ID NO:2141, and a light chain having an amino acid sequence having at least 95% identity to an amino acid sequence of SEQ ID NO:2142.

In one aspect, provided herein is an antibody that binds TRDV2, comprising: (i) a VH comprising a VH CDR1, a VH CDR2, and a VH CDR3 having an amino acid sequence of SEQ ID NOs:2143, 2144, and 2145, respectively, and (ii) a VL comprising a VL CDR1, VL CDR2, and VL CDR3 having an amino acid sequence of SEQ ID NOs:2146, 2147, and 2148, respectively. In one aspect, provided herein is an antibody that binds TRDV2, comprising: (i) a VH comprising a VH CDR1, a VH CDR2, and a VH CDR3 having an amino acid sequence of SEQ ID NOs:2149, 2150, and 2151, respectively, and (ii) a VL comprising a VL CDR1, VL CDR2, and VL CDR3 having an amino acid sequence of SEQ ID NOs:2152, 2153, and 2154, respectively. In one aspect, provided herein is an antibody that binds TRDV2, comprising: (i) a VH comprising a VH CDR1, a VH CDR2, and a VH CDR3 having an amino acid sequence of SEQ ID NOs:2155, 2156, and 2157, respectively, and (ii) a VL comprising a VL CDR1, VL CDR2, and VL CDR3 having an amino acid sequence of SEQ ID NOs:2158, 2159, and 2160, respectively. In one aspect, provided herein is an antibody that binds TRDV2, comprising: (i) a VH comprising a VH CDR1, a VH CDR2, and a VH CDR3 having an amino acid sequence of SEQ ID NOs:2161, 2162, and 2163, respectively, and (ii) a VL comprising a VL CDR1, VL CDR2, and VL CDR3 having an amino acid sequence of SEQ ID NOs:2164, 2165, and 2166, respectively. In one aspect, provided herein is an antibody that binds TRDV2, comprising: (i) a VH comprising a VH CDR1, a VH CDR2, and a VH CDR3 having an amino acid sequence of SEQ ID NOs:2167, 2168, and 2169, respectively, and (ii) a VL comprising a VL CDR1, VL CDR2, and VL CDR3 having an amino acid sequence of SEQ ID NOs:2170, 2171, and 2172, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2173; and (ii) a VL comprising a VL CDR1, a VL CDR2, and a VL CDR3 having an amino acid sequence of a VL CDR1, a VL CDR2, and a VL CDR3, respectively, of SEQ ID NO:2174. In one aspect, provided herein is an antibody that binds TRDV2, comprising a VH having an amino acid sequence of SEQ ID NO:2173. In one aspect, provided herein is an antibody that binds TRDV2, comprising a VL having an amino acid sequence of SEQ ID NO:2174. In one aspect, provided herein is an antibody that binds TRDV2, comprising a VH having an amino acid sequence of SEQ ID NO:2173, and a VL having an amino acid sequence of SEQ ID NO:2174. In one aspect, provided herein is an antibody that binds TRDV2, comprising a heavy chain having an amino acid sequence of SEQ ID NO:2175. In one aspect, provided herein is an antibody that binds TRDV2, comprising a light chain having an amino acid sequence of SEQ ID NO:2176. In one aspect, provided herein is an antibody that binds TRDV2, comprising a heavy chain having an amino acid sequence of SEQ ID NO:2175, and a light chain having an amino acid sequence of SEQ ID NO:2176. In one aspect, provided herein is an antibody that binds TRDV2, comprising a VH having an amino acid sequence having at least 95% identity to an amino acid sequence of SEQ ID NO:2173. In one aspect, provided herein is an antibody that binds TRDV2, comprising a VL having an amino acid sequence having at least 95% identity to an amino acid sequence of SEQ ID NO:2174. In one aspect, provided herein is an antibody that binds TRDV2, comprising a VH having an amino acid sequence having at least 95% identity to an amino acid sequence of SEQ ID NO:2173, and a VL having an amino acid sequence having at least 95% identity to an amino acid sequence of SEQ ID NO:2174. In one aspect, provided herein is an antibody that binds TRDV2, comprising a heavy chain having an amino acid sequence having at least 95% identity to an amino acid sequence of SEQ ID NO:2175. In one aspect, provided herein is an antibody that binds TRDV2, comprising a light chain having an amino acid sequence having at least 95% identity to an amino acid sequence of SEQ ID NO:2176. In one aspect, provided herein is an antibody that binds TRDV2, comprising a heavy chain having an amino acid sequence having at least 95% identity to an amino acid sequence of SEQ ID NO:2175, and a light chain having an amino acid sequence having at least 95% identity to an amino acid sequence of SEQ ID NO:2176.

In one aspect, provided herein is an antibody that binds TRDV2, comprising: (i) a VH comprising a VH CDR1, a VH CDR2, and a VH CDR3 having an amino acid sequence of SEQ ID NOs:2177, 2178, and 2179, respectively, and (ii) a VL comprising a VL CDR1, VL CDR2, and VL CDR3 having an amino acid sequence of SEQ ID NOs:2180, 2181, and 2182, respectively. In one aspect, provided herein is an antibody that binds TRDV2, comprising: (i) a VH comprising a VH CDR1, a VH CDR2, and a VH CDR3 having an amino acid sequence of SEQ ID NOs:2183, 2184, and 2185, respectively, and (ii) a VL comprising a VL CDR1, VL CDR2, and VL CDR3 having an amino acid sequence of SEQ ID NOs:2186, 2187, and 2188, respectively. In one aspect, provided herein is an antibody that binds TRDV2, comprising: (i) a VH comprising a VH CDR1, a VH CDR2, and a VH CDR3 having an amino acid sequence of SEQ ID NOs:2189, 2190, and 2191, respectively, and (ii) a VL comprising a VL CDR1, VL CDR2, and VL CDR3 having an amino acid sequence of SEQ ID NOs:2192, 2193, and 2194, respectively. In one aspect, provided herein is an antibody that binds TRDV2, comprising: (i) a VH comprising a VH CDR1, a VH CDR2, and a VH CDR3 having an amino acid sequence of SEQ ID NOs:2195, 2196, and 2197, respectively, and (ii) a VL comprising a VL CDR1, VL CDR2, and VL CDR3 having an amino acid sequence of SEQ ID NOs:2198, 2199, and 2200, respectively. In one aspect, provided herein is an antibody that binds TRDV2, comprising: (i) a VH comprising a VH CDR1, a VH CDR2, and a VH CDR3 having an amino acid sequence of SEQ ID NOs:2201, 2202, and 2203, respectively, and (ii) a VL comprising a VL CDR1, VL CDR2, and VL CDR3 having an amino acid sequence of SEQ ID NOs:2204, 2205, and 2206, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2207; and (ii) a VL comprising a VL CDR1, a VL CDR2, and a VL CDR3 having an amino acid sequence of a VL CDR1, a VL CDR2, and a VL CDR3, respectively, of SEQ ID NO:2208. In one aspect, provided herein is an antibody that binds TRDV2, comprising a VH having an amino acid sequence of SEQ ID NO:2207. In one aspect, provided herein is an antibody that binds TRDV2, comprising a VL having an amino acid sequence of SEQ ID NO:2208. In one aspect, provided herein is an antibody that binds TRDV2, comprising a VH having an amino acid sequence of SEQ ID NO:2207, and a VL having an amino acid sequence of SEQ ID NO:2208. In one aspect, provided herein is an antibody that binds TRDV2, comprising a heavy chain having an amino acid sequence of SEQ ID NO:2209. In one aspect, provided herein is an antibody that binds TRDV2, comprising a light chain having an amino acid sequence of SEQ ID NO:2210. In one aspect, provided herein is an antibody that binds TRDV2, comprising a heavy chain having an amino acid sequence of SEQ ID NO:2209, and a light chain having an amino acid sequence of SEQ ID NO:2210. In one aspect, provided herein is an antibody that binds TRDV2, comprising a VH having an amino acid sequence having at least 95% identity to an amino acid sequence of SEQ ID NO:2207. In one aspect, provided herein is an antibody that binds TRDV2, comprising a VL having an amino acid sequence having at least 95% identity to an amino acid sequence of SEQ ID NO:2208. In one aspect, provided herein is an antibody that binds TRDV2, comprising a VH having an amino acid sequence having at least 95% identity to an amino acid sequence of SEQ ID NO:2207, and a VL having an amino acid sequence having at least 95% identity to an amino acid sequence of SEQ ID NO:2208. In one aspect, provided herein is an antibody that binds TRDV2, comprising a heavy chain having an amino acid sequence having at least 95% identity to an amino acid sequence of SEQ ID NO:2209. In one aspect, provided herein is an antibody that binds TRDV2, comprising a light chain having an amino acid sequence having at least 95% identity to an amino acid sequence of SEQ ID NO:2210. In one aspect, provided herein is an antibody that binds TRDV2, comprising a heavy chain having an amino acid sequence having at least 95% identity to an amino acid sequence of SEQ ID NO:2209, and a light chain having an amino acid sequence having at least 95% identity to an amino acid sequence of SEQ ID NO:2210.

In one aspect, provided herein is an antibody that binds TRDV2, comprising: (i) a VH comprising a VH CDR1, a VH CDR2, and a VH CDR3 having an amino acid sequence of SEQ ID NOs:2211, 2212, and 2213, respectively, and (ii) a VL comprising a VL CDR1, VL CDR2, and VL CDR3 having an amino acid sequence of SEQ ID NOs:2214, 2215, and 2216, respectively. In one aspect, provided herein is an antibody that binds TRDV2, comprising: (i) a VH comprising a VH CDR1, a VH CDR2, and a VH CDR3 having an amino acid sequence of SEQ ID NOs:2217, 2218, and 2219, respectively, and (ii) a VL comprising a VL CDR1, VL CDR2, and VL CDR3 having an amino acid sequence of SEQ ID NOs:2220, 2221, and 2222, respectively. In one aspect, provided herein is an antibody that binds TRDV2, comprising: (i) a VH comprising a VH CDR1, a VH CDR2, and a VH CDR3 having an amino acid sequence of SEQ ID NOs:2223, 2224, and 2225, respectively, and (ii) a VL comprising a VL CDR1, VL CDR2, and VL CDR3 having an amino acid sequence of SEQ ID NOs:2226, 2227, and 2228, respectively. In one aspect, provided herein is an antibody that binds TRDV2, comprising: (i) a VH comprising a VH CDR1, a VH CDR2, and a VH CDR3 having an amino acid sequence of SEQ ID NOs:2229, 2230, and 2231, respectively, and (ii) a VL comprising a VL CDR1, VL CDR2, and VL CDR3 having an amino acid sequence of SEQ ID NOs:2232, 2233, and 2234, respectively. In one aspect, provided herein is an antibody that binds TRDV2, comprising: (i) a VH comprising a VH CDR1, a VH CDR2, and a VH CDR3 having an amino acid sequence of SEQ ID NOs:2235, 2236, and 2237, respectively, and (ii) a VL comprising a VL CDR1, VL CDR2, and VL CDR3 having an amino acid sequence of SEQ ID NOs:2238, 2239, and 2240, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2241; and (ii) a VL comprising a VL CDR1, a VL CDR2, and a VL CDR3 having an amino acid sequence of a VL CDR1, a VL CDR2, and a VL CDR3, respectively, of SEQ ID NO:2242. In one aspect, provided herein is an antibody that binds TRDV2, comprising a VH having an amino acid sequence of SEQ ID NO:2241. In one aspect, provided herein is an antibody that binds TRDV2, comprising a VL having an amino acid sequence of SEQ ID NO:2242. In one aspect, provided herein is an antibody that binds TRDV2, comprising a VH having an amino acid sequence of SEQ ID NO:2241, and a VL having an amino acid sequence of SEQ ID NO:2242. In one aspect, provided herein is an antibody that binds TRDV2, comprising a heavy chain having an amino acid sequence of SEQ ID NO:2243. In one aspect, provided herein is an antibody that binds TRDV2, comprising a light chain having an amino acid sequence of SEQ ID NO:2244. In one aspect, provided herein is an antibody that binds TRDV2, comprising a heavy chain having an amino acid sequence of SEQ ID NO:2243, and a light chain having an amino acid sequence of SEQ ID NO:2244. In one aspect, provided herein is an antibody that binds TRDV2, comprising a VH having an amino acid sequence having at least 95% identity to an amino acid sequence of SEQ ID NO:2241. In one aspect, provided herein is an antibody that binds TRDV2, comprising a VL having an amino acid sequence having at least 95% identity to an amino acid sequence of SEQ ID NO:2242. In one aspect, provided herein is an antibody that binds TRDV2, comprising a VH having an amino acid sequence having at least 95% identity to an amino acid sequence of SEQ ID NO:2241, and a VL having an amino acid sequence having at least 95% identity to an amino acid sequence of SEQ ID NO:2242. In one aspect, provided herein is an antibody that binds TRDV2, comprising a heavy chain having an amino acid sequence having at least 95% identity to an amino acid sequence of SEQ ID NO:2243. In one aspect, provided herein is an antibody that binds TRDV2, comprising a light chain having an amino acid sequence having at least 95% identity to an amino acid sequence of SEQ ID NO:2244. In one aspect, provided herein is an antibody that binds TRDV2, comprising a heavy chain having an amino acid sequence having at least 95% identity to an amino acid sequence of SEQ ID NO:2243, and a light chain having an amino acid sequence having at least 95% identity to an amino acid sequence of SEQ ID NO:2244.

In one aspect, provided herein is an antibody that binds TRDV2, comprising: (i) a VH comprising a VH CDR1, a VH CDR2, and a VH CDR3 having an amino acid sequence of SEQ ID NOs:2245, 2246, and 2247, respectively, and (ii) a VL comprising a VL CDR1, VL CDR2, and VL CDR3 having an amino acid sequence of SEQ ID NOs:2248, 2249, and 2250, respectively. In one aspect, provided herein is an antibody that binds TRDV2, comprising: (i) a VH comprising a VH CDR1, a VH CDR2, and a VH CDR3 having an amino acid sequence of SEQ ID NOs:2251, 2252, and 2253, respectively, and (ii) a VL comprising a VL CDR1, VL CDR2, and VL CDR3 having an amino acid sequence of SEQ ID NOs:2254, 2255, and 2256, respectively. In one aspect, provided herein is an antibody that binds TRDV2, comprising: (i) a VH comprising a VH CDR1, a VH CDR2, and a VH CDR3 having an amino acid sequence of SEQ ID NOs:2257, 2258, and 2259, respectively, and (ii) a VL comprising a VL CDR1, VL CDR2, and VL CDR3 having an amino acid sequence of SEQ ID NOs:2260, 2261, and 2262, respectively. In one aspect, provided herein is an antibody that binds TRDV2, comprising: (i) a VH comprising a VH CDR1, a VH CDR2, and a VH CDR3 having an amino acid sequence of SEQ ID NOs:2263, 2264, and 2265, respectively, and (ii) a VL comprising a VL CDR1, VL CDR2, and VL CDR3 having an amino acid sequence of SEQ ID NOs:2266, 2267, and 2268, respectively. In one aspect, provided herein is an antibody that binds TRDV2, comprising: (i) a VH comprising a VH CDR1, a VH CDR2, and a VH CDR3 having an amino acid sequence of SEQ ID NOs:2269, 2270, and 2271, respectively, and (ii) a VL comprising a VL CDR1, VL CDR2, and VL CDR3 having an amino acid sequence of SEQ ID NOs:2272, 2273, and 2274, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2275; and (ii) a VL comprising a VL CDR1, a VL CDR2, and a VL CDR3 having an amino acid sequence of a VL CDR1, a VL CDR2, and a VL CDR3, respectively, of SEQ ID NO:2276. In one aspect, provided herein is an antibody that binds TRDV2, comprising a VH having an amino acid sequence of SEQ ID NO:2275. In one aspect, provided herein is an antibody that binds TRDV2, comprising a VL having an amino acid sequence of SEQ ID NO:2276. In one aspect, provided herein is an antibody that binds TRDV2, comprising a VH having an amino acid sequence of SEQ ID NO:2275, and a VL having an amino acid sequence of SEQ ID NO:2276. In one aspect, provided herein is an antibody that binds TRDV2, comprising a heavy chain having an amino acid sequence of SEQ ID NO:2277. In one aspect, provided herein is an antibody that binds TRDV2, comprising a light chain having an amino acid sequence of SEQ ID NO:2278. In one aspect, provided herein is an antibody that binds TRDV2, comprising a heavy chain having an amino acid sequence of SEQ ID NO:2277, and a light chain having an amino acid sequence of SEQ ID NO:2278. In one aspect, provided herein is an antibody that binds TRDV2, comprising a VH having an amino acid sequence having at least 95% identity to an amino acid sequence of SEQ ID NO:2275. In one aspect, provided herein is an antibody that binds TRDV2, comprising a VL having an amino acid sequence having at least 95% identity to an amino acid sequence of SEQ ID NO:2276. In one aspect, provided herein is an antibody that binds TRDV2, comprising a VH having an amino acid sequence having at least 95% identity to an amino acid sequence of SEQ ID NO:2275, and a VL having an amino acid sequence having at least 95% identity to an amino acid sequence of SEQ ID NO:2276. In one aspect, provided herein is an antibody that binds TRDV2, comprising a heavy chain having an amino acid sequence having at least 95% identity to an amino acid sequence of SEQ ID NO:2277. In one aspect, provided herein is an antibody that binds TRDV2, comprising a light chain having an amino acid sequence having at least 95% identity to an amino acid sequence of SEQ ID NO:2278. In one aspect, provided herein is an antibody that binds TRDV2, comprising a heavy chain having an amino acid sequence having at least 95% identity to an amino acid sequence of SEQ ID NO:2277, and a light chain having an amino acid sequence having at least 95% identity to an amino acid sequence of SEQ ID NO:2278.

In one aspect, provided herein is an antibody that binds TRDV2, comprising: (i) a VH comprising a VH CDR1, a VH CDR2, and a VH CDR3 having an amino acid sequence of SEQ ID NOs:2279, 2280, and 2281, respectively, and (ii) a VL comprising a VL CDR1, VL CDR2, and VL CDR3 having an amino acid sequence of SEQ ID NOs:2282, 2283, and 2284, respectively. In one aspect, provided herein is an antibody that binds TRDV2, comprising: (i) a VH comprising a VH CDR1, a VH CDR2, and a VH CDR3 having an amino acid sequence of SEQ ID NOs:2285, 2286, and 2287, respectively, and (ii) a VL comprising a VL CDR1, VL CDR2, and VL CDR3 having an amino acid sequence of SEQ ID NOs:2288, 2289, and 2290, respectively. In one aspect, provided herein is an antibody that binds TRDV2, comprising: (i) a VH comprising a VH CDR1, a VH CDR2, and a VH CDR3 having an amino acid sequence of SEQ ID NOs:2291, 2292, and 2293, respectively, and (ii) a VL comprising a VL CDR1, VL CDR2, and VL CDR3 having an amino acid sequence of SEQ ID NOs:2294, 2295, and 2296, respectively. In one aspect, provided herein is an antibody that binds TRDV2, comprising: (i) a VH comprising a VH CDR1, a VH CDR2, and a VH CDR3 having an amino acid sequence of SEQ ID NOs:2297, 2298, and 2299, respectively, and (ii) a VL comprising a VL CDR1, VL CDR2, and VL CDR3 having an amino acid sequence of SEQ ID NOs:2300, 2301, and 2302, respectively. In one aspect, provided herein is an antibody that binds TRDV2, comprising: (i) a VH comprising a VH CDR1, a VH CDR2, and a VH CDR3 having an amino acid sequence of SEQ ID NOs:2303, 2304, and 2305, respectively, and (ii) a VL comprising a VL CDR1, VL CDR2, and VL CDR3 having an amino acid sequence of SEQ ID NOs:2306, 2307, and 2308, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2309; and (ii) a VL comprising a VL CDR1, a VL CDR2, and a VL CDR3 having an amino acid sequence of a VL CDR1, a VL CDR2, and a VL CDR3, respectively, of SEQ ID NO:2310. In one aspect, provided herein is an antibody that binds TRDV2, comprising a VH having an amino acid sequence of SEQ ID NO:2309. In one aspect, provided herein is an antibody that binds TRDV2, comprising a VL having an amino acid sequence of SEQ ID NO:2310. In one aspect, provided herein is an antibody that binds TRDV2, comprising a VH having an amino acid sequence of SEQ ID NO:2309, and a VL having an amino acid sequence of SEQ ID NO:2310. In one aspect, provided herein is an antibody that binds TRDV2, comprising a heavy chain having an amino acid sequence of SEQ ID NO:2311. In one aspect, provided herein is an antibody that binds TRDV2, comprising a light chain having an amino acid sequence of SEQ ID NO:2312. In one aspect, provided herein is an antibody that binds TRDV2, comprising a heavy chain having an amino acid sequence of SEQ ID NO:2311, and a light chain having an amino acid sequence of SEQ ID NO:2312. In one aspect, provided herein is an antibody that binds TRDV2, comprising a VH having an amino acid sequence having at least 95% identity to an amino acid sequence of SEQ ID NO:2309. In one aspect, provided herein is an antibody that binds TRDV2, comprising a VL having an amino acid sequence having at least 95% identity to an amino acid sequence of SEQ ID NO:2310. In one aspect, provided herein is an antibody that binds TRDV2, comprising a VH having an amino acid sequence having at least 95% identity to an amino acid sequence of SEQ ID NO:2309, and a VL having an amino acid sequence having at least 95% identity to an amino acid sequence of SEQ ID NO:2310. In one aspect, provided herein is an antibody that binds TRDV2, comprising a heavy chain having an amino acid sequence having at least 95% identity to an amino acid sequence of SEQ ID NO:2311. In one aspect, provided herein is an antibody that binds TRDV2, comprising a light chain having an amino acid sequence having at least 95% identity to an amino acid sequence of SEQ ID NO:2312. In one aspect, provided herein is an antibody that binds TRDV2, comprising a heavy chain having an amino acid sequence having at least 95% identity to an amino acid sequence of SEQ ID NO:2311, and a light chain having an amino acid sequence having at least 95% identity to an amino acid sequence of SEQ ID NO:2312.

In one aspect, provided herein is an antibody that binds TRDV2, comprising: (i) a VH comprising a VH CDR1, a VH CDR2, and a VH CDR3 having an amino acid sequence of SEQ ID NOs:2313, 2314, and 2315, respectively, and (ii) a VL comprising a VL CDR1, VL CDR2, and VL CDR3 having an amino acid sequence of SEQ ID NOs:2316, 2317, and 2318, respectively. In one aspect, provided herein is an antibody that binds TRDV2, comprising: (i) a VH comprising a VH CDR1, a VH CDR2, and a VH CDR3 having an amino acid sequence of SEQ ID NOs:2319, 2320, and 2321, respectively, and (ii) a VL comprising a VL CDR1, VL CDR2, and VL CDR3 having an amino acid sequence of SEQ ID NOs:2322, 2323, and 2324, respectively. In one aspect, provided herein is an antibody that binds TRDV2, comprising: (i) a VH comprising a VH CDR1, a VH CDR2, and a VH CDR3 having an amino acid sequence of SEQ ID NOs:2325, 2326, and 2327, respectively, and (ii) a VL comprising a VL CDR1, VL CDR2, and VL CDR3 having an amino acid sequence of SEQ ID NOs:2328, 2329, and 2330, respectively. In one aspect, provided herein is an antibody that binds TRDV2, comprising: (i) a VH comprising a VH CDR1, a VH CDR2, and a VH CDR3 having an amino acid sequence of SEQ ID NOs:2331, 2332, and 2333, respectively, and (ii) a VL comprising a VL CDR1, VL CDR2, and VL CDR3 having an amino acid sequence of SEQ ID NOs:2334, 2335, and 2336, respectively. In one aspect, provided herein is an antibody that binds TRDV2, comprising: (i) a VH comprising a VH CDR1, a VH CDR2, and a VH CDR3 having an amino acid sequence of SEQ ID NOs:2337, 2338, and 2339, respectively, and (ii) a VL comprising a VL CDR1, VL CDR2, and VL CDR3 having an amino acid sequence of SEQ ID NOs:2340, 2341, and 2342, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2343; and (ii) a VL comprising a VL CDR1, a VL CDR2, and a VL CDR3 having an amino acid sequence of a VL CDR1, a VL CDR2, and a VL CDR3, respectively, of SEQ ID NO:2344. In one aspect, provided herein is an antibody that binds TRDV2, comprising a VH having an amino acid sequence of SEQ ID NO:2343. In one aspect, provided herein is an antibody that binds TRDV2, comprising a VL having an amino acid sequence of SEQ ID NO:2344. In one aspect, provided herein is an antibody that binds TRDV2, comprising a VH having an amino acid sequence of SEQ ID NO:2343, and a VL having an amino acid sequence of SEQ ID NO:2344. In one aspect, provided herein is an antibody that binds TRDV2, comprising a heavy chain having an amino acid sequence of SEQ ID NO:2345. In one aspect, provided herein is an antibody that binds TRDV2, comprising a light chain having an amino acid sequence of SEQ ID NO:2346. In one aspect, provided herein is an antibody that binds TRDV2, comprising a heavy chain having an amino acid sequence of SEQ ID NO:2345, and a light chain having an amino acid sequence of SEQ ID NO:2346. In one aspect, provided herein is an antibody that binds TRDV2, comprising a VH having an amino acid sequence having at least 95% identity to an amino acid sequence of SEQ ID NO:2343. In one aspect, provided herein is an antibody that binds TRDV2, comprising a VL having an amino acid sequence having at least 95% identity to an amino acid sequence of SEQ ID NO:2344. In one aspect, provided herein is an antibody that binds TRDV2, comprising a VH having an amino acid sequence having at least 95% identity to an amino acid sequence of SEQ ID NO:2343, and a VL having an amino acid sequence having at least 95% identity to an amino acid sequence of SEQ ID NO:2344. In one aspect, provided herein is an antibody that binds TRDV2, comprising a heavy chain having an amino acid sequence having at least 95% identity to an amino acid sequence of SEQ ID NO:2345. In one aspect, provided herein is an antibody that binds TRDV2, comprising a light chain having an amino acid sequence having at least 95% identity to an amino acid sequence of SEQ ID NO:2346. In one aspect, provided herein is an antibody that binds TRDV2, comprising a heavy chain having an amino acid sequence having at least 95% identity to an amino acid sequence of SEQ ID NO:2345, and a light chain having an amino acid sequence having at least 95% identity to an amino acid sequence of SEQ ID NO:2346.

In one aspect, provided herein is an antibody that binds TRDV2, comprising: (i) a VH comprising a VH CDR1, a VH CDR2, and a VH CDR3 having an amino acid sequence of SEQ ID NOs:2347, 2348, and 2349, respectively, and (ii) a VL comprising a VL CDR1, VL CDR2, and VL CDR3 having an amino acid sequence of SEQ ID NOs:2350, 2351, and 2352, respectively. In one aspect, provided herein is an antibody that binds TRDV2, comprising: (i) a VH comprising a VH CDR1, a VH CDR2, and a VH CDR3 having an amino acid sequence of SEQ ID NOs:2353, 2354, and 2355, respectively, and (ii) a VL comprising a VL CDR1, VL CDR2, and VL CDR3 having an amino acid sequence of SEQ ID NOs:2356, 2357, and 2358, respectively. In one aspect, provided herein is an antibody that binds TRDV2, comprising: (i) a VH comprising a VH CDR1, a VH CDR2, and a VH CDR3 having an amino acid sequence of SEQ ID NOs:2359, 2360, and 2361, respectively, and (ii) a VL comprising a VL CDR1, VL CDR2, and VL CDR3 having an amino acid sequence of SEQ ID NOs:2362, 2363, and 2364, respectively. In one aspect, provided herein is an antibody that binds TRDV2, comprising: (i) a VH comprising a VH CDR1, a VH CDR2, and a VH CDR3 having an amino acid sequence of SEQ ID NOs:2365, 2366, and 2367, respectively, and (ii) a VL comprising a VL CDR1, VL CDR2, and VL CDR3 having an amino acid sequence of SEQ ID NOs:2368, 2369, and 2370, respectively. In one aspect, provided herein is an antibody that binds TRDV2, comprising: (i) a VH comprising a VH CDR1, a VH CDR2, and a VH CDR3 having an amino acid sequence of SEQ ID NOs:2371, 2372, and 2373, respectively, and (ii) a VL comprising a VL CDR1, VL CDR2, and VL CDR3 having an amino acid sequence of SEQ ID NOs:2374, 2375, and 2376, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2377; and (ii) a VL comprising a VL CDR1, a VL CDR2, and a VL CDR3 having an amino acid sequence of a VL CDR1, a VL CDR2, and a VL CDR3, respectively, of SEQ ID NO:2378. In one aspect, provided herein is an antibody that binds TRDV2, comprising a VH having an amino acid sequence of SEQ ID NO:2377. In one aspect, provided herein is an antibody that binds TRDV2, comprising a VL having an amino acid sequence of SEQ ID NO:2378. In one aspect, provided herein is an antibody that binds TRDV2, comprising a VH having an amino acid sequence of SEQ ID NO:2377, and a VL having an amino acid sequence of SEQ ID NO:2378. In one aspect, provided herein is an antibody that binds TRDV2, comprising a heavy chain having an amino acid sequence of SEQ ID NO:2379. In one aspect, provided herein is an antibody that binds TRDV2, comprising a light chain having an amino acid sequence of SEQ ID NO:2380. In one aspect, provided herein is an antibody that binds TRDV2, comprising a heavy chain having an amino acid sequence of SEQ ID NO:2379, and a light chain having an amino acid sequence of SEQ ID NO:2380. In one aspect, provided herein is an antibody that binds TRDV2, comprising a VH having an amino acid sequence having at least 95% identity to an amino acid sequence of SEQ ID NO:2377. In one aspect, provided herein is an antibody that binds TRDV2, comprising a VL having an amino acid sequence having at least 95% identity to an amino acid sequence of SEQ ID NO:2378. In one aspect, provided herein is an antibody that binds TRDV2, comprising a VH having an amino acid sequence having at least 95% identity to an amino acid sequence of SEQ ID NO:2377, and a VL having an amino acid sequence having at least 95% identity to an amino acid sequence of SEQ ID NO:2378. In one aspect, provided herein is an antibody that binds TRDV2, comprising a heavy chain having an amino acid sequence having at least 95% identity to an amino acid sequence of SEQ ID NO:2379. In one aspect, provided herein is an antibody that binds TRDV2, comprising a light chain having an amino acid sequence having at least 95% identity to an amino acid sequence of SEQ ID NO:2380. In one aspect, provided herein is an antibody that binds TRDV2, comprising a heavy chain having an amino acid sequence having at least 95% identity to an amino acid sequence of SEQ ID NO:2379, and a light chain having an amino acid sequence having at least 95% identity to an amino acid sequence of SEQ ID NO:2380.

In one aspect, provided herein is an antibody that binds TRDV2, comprising: (i) a VH comprising a VH CDR1, a VH CDR2, and a VH CDR3 having an amino acid sequence of SEQ ID NOs:2381, 2382, and 2383, respectively, and (ii) a VL comprising a VL CDR1, VL CDR2, and VL CDR3 having an amino acid sequence of SEQ ID NOs:2384, 2385, and 2386, respectively. In one aspect, provided herein is an antibody that binds TRDV2, comprising: (i) a VH comprising a VH CDR1, a VH CDR2, and a VH CDR3 having an amino acid sequence of SEQ ID NOs:2387, 2388, and 2389, respectively, and (ii) a VL comprising a VL CDR1, VL CDR2, and VL CDR3 having an amino acid sequence of SEQ ID NOs:2390, 2391, and 2392, respectively. In one aspect, provided herein is an antibody that binds TRDV2, comprising: (i) a VH comprising a VH CDR1, a VH CDR2, and a VH CDR3 having an amino acid sequence of SEQ ID NOs:2393, 2394, and 2395, respectively, and (ii) a VL comprising a VL CDR1, VL CDR2, and VL CDR3 having an amino acid sequence of SEQ ID NOs:2396, 2397, and 2398, respectively. In one aspect, provided herein is an antibody that binds TRDV2, comprising: (i) a VH comprising a VH CDR1, a VH CDR2, and a VH CDR3 having an amino acid sequence of SEQ ID NOs:2399, 2400, and 2401, respectively, and (ii) a VL comprising a VL CDR1, VL CDR2, and VL CDR3 having an amino acid sequence of SEQ ID NOs:2402, 2403, and 2404, respectively. In one aspect, provided herein is an antibody that binds TRDV2, comprising: (i) a VH comprising a VH CDR1, a VH CDR2, and a VH CDR3 having an amino acid sequence of SEQ ID NOs:2405, 2406, and 2407, respectively, and (ii) a VL comprising a VL CDR1, VL CDR2, and VL CDR3 having an amino acid sequence of SEQ ID NOs:2408, 2409, and 2410, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2411; and (ii) a VL comprising a VL CDR1, a VL CDR2, and a VL CDR3 having an amino acid sequence of a VL CDR1, a VL CDR2, and a VL CDR3, respectively, of SEQ ID NO:2412. In one aspect, provided herein is an antibody that binds TRDV2, comprising a VH having an amino acid sequence of SEQ ID NO:2411. In one aspect, provided herein is an antibody that binds TRDV2, comprising a VL having an amino acid sequence of SEQ ID NO:2412. In one aspect, provided herein is an antibody that binds TRDV2, comprising a VH having an amino acid sequence of SEQ ID NO:2411, and a VL having an amino acid sequence of SEQ ID NO:2412. In one aspect, provided herein is an antibody that binds TRDV2, comprising a heavy chain having an amino acid sequence of SEQ ID NO:2413. In one aspect, provided herein is an antibody that binds TRDV2, comprising a light chain having an amino acid sequence of SEQ ID NO:2414. In one aspect, provided herein is an antibody that binds TRDV2, comprising a heavy chain having an amino acid sequence of SEQ ID NO:2413, and a light chain having an amino acid sequence of SEQ ID NO:2414. In one aspect, provided herein is an antibody that binds TRDV2, comprising a VH having an amino acid sequence having at least 95% identity to an amino acid sequence of SEQ ID NO:2411. In one aspect, provided herein is an antibody that binds TRDV2, comprising a VL having an amino acid sequence having at least 95% identity to an amino acid sequence of SEQ ID NO:2412. In one aspect, provided herein is an antibody that binds TRDV2, comprising a VH having an amino acid sequence having at least 95% identity to an amino acid sequence of SEQ ID NO:2411, and a VL having an amino acid sequence having at least 95% identity to an amino acid sequence of SEQ ID NO:2412. In one aspect, provided herein is an antibody that binds TRDV2, comprising a heavy chain having an amino acid sequence having at least 95% identity to an amino acid sequence of SEQ ID NO:2413. In one aspect, provided herein is an antibody that binds TRDV2, comprising a light chain having an amino acid sequence having at least 95% identity to an amino acid sequence of SEQ ID NO:2414. In one aspect, provided herein is an antibody that binds TRDV2, comprising a heavy chain having an amino acid sequence having at least 95% identity to an amino acid sequence of SEQ ID NO:2413, and a light chain having an amino acid sequence having at least 95% identity to an amino acid sequence of SEQ ID NO:2414.

In one aspect, provided herein is an antibody that binds TRDV2, comprising: (i) a VH comprising a VH CDR1, a VH CDR2, and a VH CDR3 having an amino acid sequence of SEQ ID NOs:2415, 2416, and 2417, respectively, and (ii) a VL comprising a VL CDR1, VL CDR2, and VL CDR3 having an amino acid sequence of SEQ ID NOs:2418, 2419, and 2420, respectively. In one aspect, provided herein is an antibody that binds TRDV2, comprising: (i) a VH comprising a VH CDR1, a VH CDR2, and a VH CDR3 having an amino acid sequence of SEQ ID NOs:2421, 2422, and 2423, respectively, and (ii) a VL comprising a VL CDR1, VL CDR2, and VL CDR3 having an amino acid sequence of SEQ ID NOs:2424, 2425, and 2426, respectively. In one aspect, provided herein is an antibody that binds TRDV2, comprising: (i) a VH comprising a VH CDR1, a VH CDR2, and a VH CDR3 having an amino acid sequence of SEQ ID NOs:2427, 2428, and 2429, respectively, and (ii) a VL comprising a VL CDR1, VL CDR2, and VL CDR3 having an amino acid sequence of SEQ ID NOs:2430, 2431, and 2432, respectively. In one aspect, provided herein is an antibody that binds TRDV2, comprising: (i) a VH comprising a VH CDR1, a VH CDR2, and a VH CDR3 having an amino acid sequence of SEQ ID NOs:2433, 2434, and 2435, respectively, and (ii) a VL comprising a VL CDR1, VL CDR2, and VL CDR3 having an amino acid sequence of SEQ ID NOs:2436, 2437, and 2438, respectively. In one aspect, provided herein is an antibody that binds TRDV2, comprising: (i) a VH comprising a VH CDR1, a VH CDR2, and a VH CDR3 having an amino acid sequence of SEQ ID NOs:2439, 2440, and 2441, respectively, and (ii) a VL comprising a VL CDR1, VL CDR2, and VL CDR3 having an amino acid sequence of SEQ ID NOs:2442, 2443, and 2444, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2445; and (ii) a VL comprising a VL CDR1, a VL CDR2, and a VL CDR3 having an amino acid sequence of a VL CDR1, a VL CDR2, and a VL CDR3, respectively, of SEQ ID NO:2446. In one aspect, provided herein is an antibody that binds TRDV2, comprising a VH having an amino acid sequence of SEQ ID NO:2445. In one aspect, provided herein is an antibody that binds TRDV2, comprising a VL having an amino acid sequence of SEQ ID NO:2446. In one aspect, provided herein is an antibody that binds TRDV2, comprising a VH having an amino acid sequence of SEQ ID NO:2445, and a VL having an amino acid sequence of SEQ ID NO:2446. In one aspect, provided herein is an antibody that binds TRDV2, comprising a heavy chain having an amino acid sequence of SEQ ID NO:2447. In one aspect, provided herein is an antibody that binds TRDV2, comprising a light chain having an amino acid sequence of SEQ ID NO:2448. In one aspect, provided herein is an antibody that binds TRDV2, comprising a heavy chain having an amino acid sequence of SEQ ID NO:2447, and a light chain having an amino acid sequence of SEQ ID NO:2448. In one aspect, provided herein is an antibody that binds TRDV2, comprising a VH having an amino acid sequence having at least 95% identity to an amino acid sequence of SEQ ID NO:2445. In one aspect, provided herein is an antibody that binds TRDV2, comprising a VL having an amino acid sequence having at least 95% identity to an amino acid sequence of SEQ ID NO:2446. In one aspect, provided herein is an antibody that binds TRDV2, comprising a VH having an amino acid sequence having at least 95% identity to an amino acid sequence of SEQ ID NO:2445, and a VL having an amino acid sequence having at least 95% identity to an amino acid sequence of SEQ ID NO:2446. In one aspect, provided herein is an antibody that binds TRDV2, comprising a heavy chain having an amino acid sequence having at least 95% identity to an amino acid sequence of SEQ ID NO:2447. In one aspect, provided herein is an antibody that binds TRDV2, comprising a light chain having an amino acid sequence having at least 95% identity to an amino acid sequence of SEQ ID NO:2448. In one aspect, provided herein is an antibody that binds TRDV2, comprising a heavy chain having an amino acid sequence having at least 95% identity to an amino acid sequence of SEQ ID NO:2447, and a light chain having an amino acid sequence having at least 95% identity to an amino acid sequence of SEQ ID NO:2448.

In one aspect, provided herein is an antibody that binds TRDV2, comprising: (i) a VH comprising a VH CDR1, a VH CDR2, and a VH CDR3 having an amino acid sequence of SEQ ID NOs:2449, 2450, and 2451, respectively, and (ii) a VL comprising a VL CDR1, VL CDR2, and VL CDR3 having an amino acid sequence of SEQ ID NOs:2452, 2453, and 2454, respectively. In one aspect, provided herein is an antibody that binds TRDV2, comprising: (i) a VH comprising a VH CDR1, a VH CDR2, and a VH CDR3 having an amino acid sequence of SEQ ID NOs:2455, 2456, and 2457, respectively, and (ii) a VL comprising a VL CDR1, VL CDR2, and VL CDR3 having an amino acid sequence of SEQ ID NOs:2458, 2459, and 2460, respectively. In one aspect, provided herein is an antibody that binds TRDV2, comprising: (i) a VH comprising a VH CDR1, a VH CDR2, and a VH CDR3 having an amino acid sequence of SEQ ID NOs:2461, 2462, and 2463, respectively, and (ii) a VL comprising a VL CDR1, VL CDR2, and VL CDR3 having an amino acid sequence of SEQ ID NOs:2464, 2465, and 2466, respectively. In one aspect, provided herein is an antibody that binds TRDV2, comprising: (i) a VH comprising a VH CDR1, a VH CDR2, and a VH CDR3 having an amino acid sequence of SEQ ID NOs:2467, 2468, and 2469, respectively, and (ii) a VL comprising a VL CDR1, VL CDR2, and VL CDR3 having an amino acid sequence of SEQ ID NOs:2470, 2471, and 2472, respectively. In one aspect, provided herein is an antibody that binds TRDV2, comprising: (i) a VH comprising a VH CDR1, a VH CDR2, and a VH CDR3 having an amino acid sequence of SEQ ID NOs:2473, 2474, and 2475, respectively, and (ii) a VL comprising a VL CDR1, VL CDR2, and VL CDR3 having an amino acid sequence of SEQ ID NOs:2476, 2477, and 2478, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2479; and (ii) a VL comprising a VL CDR1, a VL CDR2, and a VL CDR3 having an amino acid sequence of a VL CDR1, a VL CDR2, and a VL CDR3, respectively, of SEQ ID NO:2480. In one aspect, provided herein is an antibody that binds TRDV2, comprising a VH having an amino acid sequence of SEQ ID NO:2479. In one aspect, provided herein is an antibody that binds TRDV2, comprising a VL having an amino acid sequence of SEQ ID NO:2480. In one aspect, provided herein is an antibody that binds TRDV2, comprising a VH having an amino acid sequence of SEQ ID NO:2479, and a VL having an amino acid sequence of SEQ ID NO:2480. In one aspect, provided herein is an antibody that binds TRDV2, comprising a heavy chain having an amino acid sequence of SEQ ID NO:2481. In one aspect, provided herein is an antibody that binds TRDV2, comprising a light chain having an amino acid sequence of SEQ ID NO:2482. In one aspect, provided herein is an antibody that binds TRDV2, comprising a heavy chain having an amino acid sequence of SEQ ID NO:2481, and a light chain having an amino acid sequence of SEQ ID NO:2482. In one aspect, provided herein is an antibody that binds TRDV2, comprising a VH having an amino acid sequence having at least 95% identity to an amino acid sequence of SEQ ID NO:2479. In one aspect, provided herein is an antibody that binds TRDV2, comprising a VL having an amino acid sequence having at least 95% identity to an amino acid sequence of SEQ ID NO:2480. In one aspect, provided herein is an antibody that binds TRDV2, comprising a VH having an amino acid sequence having at least 95% identity to an amino acid sequence of SEQ ID NO:2479, and a VL having an amino acid sequence having at least 95% identity to an amino acid sequence of SEQ ID NO:2480. In one aspect, provided herein is an antibody that binds TRDV2, comprising a heavy chain having an amino acid sequence having at least 95% identity to an amino acid sequence of SEQ ID NO:2481. In one aspect, provided herein is an antibody that binds TRDV2, comprising a light chain having an amino acid sequence having at least 95% identity to an amino acid sequence of SEQ ID NO:2482. In one aspect, provided herein is an antibody that binds TRDV2, comprising a heavy chain having an amino acid sequence having at least 95% identity to an amino acid sequence of SEQ ID NO:2481, and a light chain having an amino acid sequence having at least 95% identity to an amino acid sequence of SEQ ID NO:2482.

In one aspect, provided herein is an antibody that binds TRDV2, comprising: (i) a VH comprising a VH CDR1, a VH CDR2, and a VH CDR3 having an amino acid sequence of SEQ ID NOs:2483, 2484, and 2485, respectively, and (ii) a VL comprising a VL CDR1, VL CDR2, and VL CDR3 having an amino acid sequence of SEQ ID NOs:2486, 2487, and 2488, respectively. In one aspect, provided herein is an antibody that binds TRDV2, comprising: (i) a VH comprising a VH CDR1, a VH CDR2, and a VH CDR3 having an amino acid sequence of SEQ ID NOs:2489, 2490, and 2491, respectively, and (ii) a VL comprising a VL CDR1, VL CDR2, and VL CDR3 having an amino acid sequence of SEQ ID NOs:2492, 2493, and 2494, respectively. In one aspect, provided herein is an antibody that binds TRDV2, comprising: (i) a VH comprising a VH CDR1, a VH CDR2, and a VH CDR3 having an amino acid sequence of SEQ ID NOs:2495, 2496, and 2497, respectively, and (ii) a VL comprising a VL CDR1, VL CDR2, and VL CDR3 having an amino acid sequence of SEQ ID NOs:2498, 2499, and 2500, respectively. In one aspect, provided herein is an antibody that binds TRDV2, comprising: (i) a VH comprising a VH CDR1, a VH CDR2, and a VH CDR3 having an amino acid sequence of SEQ ID NOs:2501, 2502, and 2503, respectively, and (ii) a VL comprising a VL CDR1, VL CDR2, and VL CDR3 having an amino acid sequence of SEQ ID NOs:2504, 2505, and 2506, respectively. In one aspect, provided herein is an antibody that binds TRDV2, comprising: (i) a VH comprising a VH CDR1, a VH CDR2, and a VH CDR3 having an amino acid sequence of SEQ ID NOs:2507, 2508, and 2509, respectively, and (ii) a VL comprising a VL CDR1, VL CDR2, and VL CDR3 having an amino acid sequence of SEQ ID NOs:2510, 2511, and 2512, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2513; and (ii) a VL comprising a VL CDR1, a VL CDR2, and a VL CDR3 having an amino acid sequence of a VL CDR1, a VL CDR2, and a VL CDR3, respectively, of SEQ ID NO:2514. In one aspect, provided herein is an antibody that binds TRDV2, comprising a VH having an amino acid sequence of SEQ ID NO:2513. In one aspect, provided herein is an antibody that binds TRDV2, comprising a VL having an amino acid sequence of SEQ ID NO:2514. In one aspect, provided herein is an antibody that binds TRDV2, comprising a VH having an amino acid sequence of SEQ ID NO:2513, and a VL having an amino acid sequence of SEQ ID NO:2514. In one aspect, provided herein is an antibody that binds TRDV2, comprising a heavy chain having an amino acid sequence of SEQ ID NO:2515. In one aspect, provided herein is an antibody that binds TRDV2, comprising a light chain having an amino acid sequence of SEQ ID NO:2516. In one aspect, provided herein is an antibody that binds TRDV2, comprising a heavy chain having an amino acid sequence of SEQ ID NO:2515, and a light chain having an amino acid sequence of SEQ ID NO:2516. In one aspect, provided herein is an antibody that binds TRDV2, comprising a VH having an amino acid sequence having at least 95% identity to an amino acid sequence of SEQ ID NO:2513. In one aspect, provided herein is an antibody that binds TRDV2, comprising a VL having an amino acid sequence having at least 95% identity to an amino acid sequence of SEQ ID NO:2514. In one aspect, provided herein is an antibody that binds TRDV2, comprising a VH having an amino acid sequence having at least 95% identity to an amino acid sequence of SEQ ID NO:2513, and a VL having an amino acid sequence having at least 95% identity to an amino acid sequence of SEQ ID NO:2514. In one aspect, provided herein is an antibody that binds TRDV2, comprising a heavy chain having an amino acid sequence having at least 95% identity to an amino acid sequence of SEQ ID NO:2515. In one aspect, provided herein is an antibody that binds TRDV2, comprising a light chain having an amino acid sequence having at least 95% identity to an amino acid sequence of SEQ ID NO:2516. In one aspect, provided herein is an antibody that binds TRDV2, comprising a heavy chain having an amino acid sequence having at least 95% identity to an amino acid sequence of SEQ ID NO:2515, and a light chain having an amino acid sequence having at least 95% identity to an amino acid sequence of SEQ ID NO:2516.

In one aspect, provided herein is an antibody that binds TRDV2, comprising: (i) a VH comprising a VH CDR1, a VH CDR2, and a VH CDR3 having an amino acid sequence of SEQ ID NOs:2517, 2518, and 2519, respectively, and (ii) a VL comprising a VL CDR1, VL CDR2, and VL CDR3 having an amino acid sequence of SEQ ID NOs:2520, 2521, and 2522, respectively. In one aspect, provided herein is an antibody that binds TRDV2, comprising: (i) a VH comprising a VH CDR1, a VH CDR2, and a VH CDR3 having an amino acid sequence of SEQ ID NOs:2523, 2524, and 2525, respectively, and (ii) a VL comprising a VL CDR1, VL CDR2, and VL CDR3 having an amino acid sequence of SEQ ID NOs:2526, 2527, and 2528, respectively. In one aspect, provided herein is an antibody that binds TRDV2, comprising: (i) a VH comprising a VH CDR1, a VH CDR2, and a VH CDR3 having an amino acid sequence of SEQ ID NOs:2529, 2530, and 2531, respectively, and (ii) a VL comprising a VL CDR1, VL CDR2, and VL CDR3 having an amino acid sequence of SEQ ID NOs:2532, 2533, and 2534, respectively. In one aspect, provided herein is an antibody that binds TRDV2, comprising: (i) a VH comprising a VH CDR1, a VH CDR2, and a VH CDR3 having an amino acid sequence of SEQ ID NOs:2535, 2536, and 2537, respectively, and (ii) a VL comprising a VL CDR1, VL CDR2, and VL CDR3 having an amino acid sequence of SEQ ID NOs:2538, 2539, and 2540, respectively. In one aspect, provided herein is an antibody that binds TRDV2, comprising: (i) a VH comprising a VH CDR1, a VH CDR2, and a VH CDR3 having an amino acid sequence of SEQ ID NOs:2541, 2542, and 2543, respectively, and (ii) a VL comprising a VL CDR1, VL CDR2, and VL CDR3 having an amino acid sequence of SEQ ID NOs:2544, 2545, and 2546, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2547; and (ii) a VL comprising a VL CDR1, a VL CDR2, and a VL CDR3 having an amino acid sequence of a VL CDR1, a VL CDR2, and a VL CDR3, respectively, of SEQ ID NO:2548. In one aspect, provided herein is an antibody that binds TRDV2, comprising a VH having an amino acid sequence of SEQ ID NO:2547. In one aspect, provided herein is an antibody that binds TRDV2, comprising a VL having an amino acid sequence of SEQ ID NO:2548. In one aspect, provided herein is an antibody that binds TRDV2, comprising a VH having an amino acid sequence of SEQ ID NO:2547, and a VL having an amino acid sequence of SEQ ID NO:2548. In one aspect, provided herein is an antibody that binds TRDV2, comprising a heavy chain having an amino acid sequence of SEQ ID NO:2549. In one aspect, provided herein is an antibody that binds TRDV2, comprising a light chain having an amino acid sequence of SEQ ID NO:2550. In one aspect, provided herein is an antibody that binds TRDV2, comprising a heavy chain having an amino acid sequence of SEQ ID NO:2549, and a light chain having an amino acid sequence of SEQ ID NO:2550. In one aspect, provided herein is an antibody that binds TRDV2, comprising a VH having an amino acid sequence having at least 95% identity to an amino acid sequence of SEQ ID NO:2547. In one aspect, provided herein is an antibody that binds TRDV2, comprising a VL having an amino acid sequence having at least 95% identity to an amino acid sequence of SEQ ID NO:2548. In one aspect, provided herein is an antibody that binds TRDV2, comprising a VH having an amino acid sequence having at least 95% identity to an amino acid sequence of SEQ ID NO:2547, and a VL having an amino acid sequence having at least 95% identity to an amino acid sequence of SEQ ID NO:2548. In one aspect, provided herein is an antibody that binds TRDV2, comprising a heavy chain having an amino acid sequence having at least 95% identity to an amino acid sequence of SEQ ID NO:2549. In one aspect, provided herein is an antibody that binds TRDV2, comprising a light chain having an amino acid sequence having at least 95% identity to an amino acid sequence of SEQ ID NO:2550. In one aspect, provided herein is an antibody that binds TRDV2, comprising a heavy chain having an amino acid sequence having at least 95% identity to an amino acid sequence of SEQ ID NO:2549, and a light chain having an amino acid sequence having at least 95% identity to an amino acid sequence of SEQ ID NO:2550.

In one aspect, provided herein is an antibody that binds TRDV2, comprising: (i) a VH comprising a VH CDR1, a VH CDR2, and a VH CDR3 having an amino acid sequence of SEQ ID NOs:2551, 2552, and 2553, respectively, and (ii) a VL comprising a VL CDR1, VL CDR2, and VL CDR3 having an amino acid sequence of SEQ ID NOs:2554, 2555, and 2556, respectively. In one aspect, provided herein is an antibody that binds TRDV2, comprising: (i) a VH comprising a VH CDR1, a VH CDR2, and a VH CDR3 having an amino acid sequence of SEQ ID NOs:2557, 2558, and 2559, respectively, and (ii) a VL comprising a VL CDR1, VL CDR2, and VL CDR3 having an amino acid sequence of SEQ ID NOs:2560, 2561, and 2562, respectively. In one aspect, provided herein is an antibody that binds TRDV2, comprising: (i) a VH comprising a VH CDR1, a VH CDR2, and a VH CDR3 having an amino acid sequence of SEQ ID NOs:2563, 2564, and 2565, respectively, and (ii) a VL comprising a VL CDR1, VL CDR2, and VL CDR3 having an amino acid sequence of SEQ ID NOs:2566, 2567, and 2568, respectively. In one aspect, provided herein is an antibody that binds TRDV2, comprising: (i) a VH comprising a VH CDR1, a VH CDR2, and a VH CDR3 having an amino acid sequence of SEQ ID NOs:2569, 2570, and 2571, respectively, and (ii) a VL comprising a VL CDR1, VL CDR2, and VL CDR3 having an amino acid sequence of SEQ ID NOs:2572, 2573, and 2574, respectively. In one aspect, provided herein is an antibody that binds TRDV2, comprising: (i) a VH comprising a VH CDR1, a VH CDR2, and a VH CDR3 having an amino acid sequence of SEQ ID NOs:2575, 2576, and 2577, respectively, and (ii) a VL comprising a VL CDR1, VL CDR2, and VL CDR3 having an amino acid sequence of SEQ ID NOs:2578, 2579, and 2580, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2581; and (ii) a VL comprising a VL CDR1, a VL CDR2, and a VL CDR3 having an amino acid sequence of a VL CDR1, a VL CDR2, and a VL CDR3, respectively, of SEQ ID NO:2582. In one aspect, provided herein is an antibody that binds TRDV2, comprising a VH having an amino acid sequence of SEQ ID NO:2581. In one aspect, provided herein is an antibody that binds TRDV2, comprising a VL having an amino acid sequence of SEQ ID NO:2582. In one aspect, provided herein is an antibody that binds TRDV2, comprising a VH having an amino acid sequence of SEQ ID NO:2581, and a VL having an amino acid sequence of SEQ ID NO:2582. In one aspect, provided herein is an antibody that binds TRDV2, comprising a heavy chain having an amino acid sequence of SEQ ID NO:2583. In one aspect, provided herein is an antibody that binds TRDV2, comprising a light chain having an amino acid sequence of SEQ ID NO:2584. In one aspect, provided herein is an antibody that binds TRDV2, comprising a heavy chain having an amino acid sequence of SEQ ID NO:2583, and a light chain having an amino acid sequence of SEQ ID NO:2584. In one aspect, provided herein is an antibody that binds TRDV2, comprising a VH having an amino acid sequence having at least 95% identity to an amino acid sequence of SEQ ID NO:2581. In one aspect, provided herein is an antibody that binds TRDV2, comprising a VL having an amino acid sequence having at least 95% identity to an amino acid sequence of SEQ ID NO:2582. In one aspect, provided herein is an antibody that binds TRDV2, comprising a VH having an amino acid sequence having at least 95% identity to an amino acid sequence of SEQ ID NO:2581, and a VL having an amino acid sequence having at least 95% identity to an amino acid sequence of SEQ ID NO:2582. In one aspect, provided herein is an antibody that binds TRDV2, comprising a heavy chain having an amino acid sequence having at least 95% identity to an amino acid sequence of SEQ ID NO:2583. In one aspect, provided herein is an antibody that binds TRDV2, comprising a light chain having an amino acid sequence having at least 95% identity to an amino acid sequence of SEQ ID NO:2584. In one aspect, provided herein is an antibody that binds TRDV2, comprising a heavy chain having an amino acid sequence having at least 95% identity to an amino acid sequence of SEQ ID NO:2583, and a light chain having an amino acid sequence having at least 95% identity to an amino acid sequence of SEQ ID NO:2584.

In one aspect, provided herein is an antibody that binds TRDV2, comprising: (i) a VH comprising a VH CDR1, a VH CDR2, and a VH CDR3 having an amino acid sequence of SEQ ID NOs:2585, 2586, and 2587, respectively, and (ii) a VL comprising a VL CDR1, VL CDR2, and VL CDR3 having an amino acid sequence of SEQ ID NOs:2588, 2589, and 2590, respectively. In one aspect, provided herein is an antibody that binds TRDV2, comprising: (i) a VH comprising a VH CDR1, a VH CDR2, and a VH CDR3 having an amino acid sequence of SEQ ID NOs:2591, 2592, and 2593, respectively, and (ii) a VL comprising a VL CDR1, VL CDR2, and VL CDR3 having an amino acid sequence of SEQ ID NOs:2594, 2595, and 2596, respectively. In one aspect, provided herein is an antibody that binds TRDV2, comprising: (i) a VH comprising a VH CDR1, a VH CDR2, and a VH CDR3 having an amino acid sequence of SEQ ID NOs:2597, 2598, and 2599, respectively, and (ii) a VL comprising a VL CDR1, VL CDR2, and VL CDR3 having an amino acid sequence of SEQ ID NOs:2600, 2601, and 2602, respectively. In one aspect, provided herein is an antibody that binds TRDV2, comprising: (i) a VH comprising a VH CDR1, a VH CDR2, and a VH CDR3 having an amino acid sequence of SEQ ID NOs:2603, 2604, and 2605, respectively, and (ii) a VL comprising a VL CDR1, VL CDR2, and VL CDR3 having an amino acid sequence of SEQ ID NOs:2606, 2607, and 2608, respectively. In one aspect, provided herein is an antibody that binds TRDV2, comprising: (i) a VH comprising a VH CDR1, a VH CDR2, and a VH CDR3 having an amino acid sequence of SEQ ID NOs:2609, 2610, and 2611, respectively, and (ii) a VL comprising a VL CDR1, VL CDR2, and VL CDR3 having an amino acid sequence of SEQ ID NOs:2612, 2613, and 2614, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2615; and (ii) a VL comprising a VL CDR1, a VL CDR2, and a VL CDR3 having an amino acid sequence of a VL CDR1, a VL CDR2, and a VL CDR3, respectively, of SEQ ID NO:2616. In one aspect, provided herein is an antibody that binds TRDV2, comprising a VH having an amino acid sequence of SEQ ID NO:2615. In one aspect, provided herein is an antibody that binds TRDV2, comprising a VL having an amino acid sequence of SEQ ID NO:2616. In one aspect, provided herein is an antibody that binds TRDV2, comprising a VH having an amino acid sequence of SEQ ID NO:2615, and a VL having an amino acid sequence of SEQ ID NO:2616. In one aspect, provided herein is an antibody that binds TRDV2, comprising a heavy chain having an amino acid sequence of SEQ ID NO:2617. In one aspect, provided herein is an antibody that binds TRDV2, comprising a light chain having an amino acid sequence of SEQ ID NO:2618. In one aspect, provided herein is an antibody that binds TRDV2, comprising a heavy chain having an amino acid sequence of SEQ ID NO:2617, and a light chain having an amino acid sequence of SEQ ID NO:2618. In one aspect, provided herein is an antibody that binds TRDV2, comprising a VH having an amino acid sequence having at least 95% identity to an amino acid sequence of SEQ ID NO:2615. In one aspect, provided herein is an antibody that binds TRDV2, comprising a VL having an amino acid sequence having at least 95% identity to an amino acid sequence of SEQ ID NO:2616. In one aspect, provided herein is an antibody that binds TRDV2, comprising a VH having an amino acid sequence having at least 95% identity to an amino acid sequence of SEQ ID NO:2615, and a VL having an amino acid sequence having at least 95% identity to an amino acid sequence of SEQ ID NO:2616. In one aspect, provided herein is an antibody that binds TRDV2, comprising a heavy chain having an amino acid sequence having at least 95% identity to an amino acid sequence of SEQ ID NO:2617. In one aspect, provided herein is an antibody that binds TRDV2, comprising a light chain having an amino acid sequence having at least 95% identity to an amino acid sequence of SEQ ID NO:2618. In one aspect, provided herein is an antibody that binds TRDV2, comprising a heavy chain having an amino acid sequence having at least 95% identity to an amino acid sequence of SEQ ID NO:2617, and a light chain having an amino acid sequence having at least 95% identity to an amino acid sequence of SEQ ID NO:2618.

In one aspect, provided herein is an antibody that binds TRDV2, comprising: (i) a VH comprising a VH CDR1, a VH CDR2, and a VH CDR3 having an amino acid sequence of SEQ ID NOs:2619, 2620, and 2621, respectively, and (ii) a VL comprising a VL CDR1, VL CDR2, and VL CDR3 having an amino acid sequence of SEQ ID NOs:2622, 2623, and 2624, respectively. In one aspect, provided herein is an antibody that binds TRDV2, comprising: (i) a VH comprising a VH CDR1, a VH CDR2, and a VH CDR3 having an amino acid sequence of SEQ ID NOs:2625, 2626, and 2627, respectively, and (ii) a VL comprising a VL CDR1, VL CDR2, and VL CDR3 having an amino acid sequence of SEQ ID NOs:2628, 2629, and 2630, respectively. In one aspect, provided herein is an antibody that binds TRDV2, comprising: (i) a VH comprising a VH CDR1, a VH CDR2, and a VH CDR3 having an amino acid sequence of SEQ ID NOs:2631, 2632, and 2633, respectively, and (ii) a VL comprising a VL CDR1, VL CDR2, and VL CDR3 having an amino acid sequence of SEQ ID NOs:2634, 2635, and 2636, respectively. In one aspect, provided herein is an antibody that binds TRDV2, comprising: (i) a VH comprising a VH CDR1, a VH CDR2, and a VH CDR3 having an amino acid sequence of SEQ ID NOs:2637, 2638, and 2639, respectively, and (ii) a VL comprising a VL CDR1, VL CDR2, and VL CDR3 having an amino acid sequence of SEQ ID NOs:2640, 2641, and 2642, respectively. In one aspect, provided herein is an antibody that binds TRDV2, comprising: (i) a VH comprising a VH CDR1, a VH CDR2, and a VH CDR3 having an amino acid sequence of SEQ ID NOs:2643, 2644, and 2645, respectively, and (ii) a VL comprising a VL CDR1, VL CDR2, and VL CDR3 having an amino acid sequence of SEQ ID NOs:2646, 2647, and 2648, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2649; and (ii) a VL comprising a VL CDR1, a VL CDR2, and a VL CDR3 having an amino acid sequence of a VL CDR1, a VL CDR2, and a VL CDR3, respectively, of SEQ ID NO:2650. In one aspect, provided herein is an antibody that binds TRDV2, comprising a VH having an amino acid sequence of SEQ ID NO:2649. In one aspect, provided herein is an antibody that binds TRDV2, comprising a VL having an amino acid sequence of SEQ ID NO:2650. In one aspect, provided herein is an antibody that binds TRDV2, comprising a VH having an amino acid sequence of SEQ ID NO:2649, and a VL having an amino acid sequence of SEQ ID NO:2650. In one aspect, provided herein is an antibody that binds TRDV2, comprising a heavy chain having an amino acid sequence of SEQ ID NO:2651. In one aspect, provided herein is an antibody that binds TRDV2, comprising a light chain having an amino acid sequence of SEQ ID NO:2652. In one aspect, provided herein is an antibody that binds TRDV2, comprising a heavy chain having an amino acid sequence of SEQ ID NO:2651, and a light chain having an amino acid sequence of SEQ ID NO:2652. In one aspect, provided herein is an antibody that binds TRDV2, comprising a VH having an amino acid sequence having at least 95% identity to an amino acid sequence of SEQ ID NO:2649. In one aspect, provided herein is an antibody that binds TRDV2, comprising a VL having an amino acid sequence having at least 95% identity to an amino acid sequence of SEQ ID NO:2650. In one aspect, provided herein is an antibody that binds TRDV2, comprising a VH having an amino acid sequence having at least 95% identity to an amino acid sequence of SEQ ID NO:2649, and a VL having an amino acid sequence having at least 95% identity to an amino acid sequence of SEQ ID NO:2650. In one aspect, provided herein is an antibody that binds TRDV2, comprising a heavy chain having an amino acid sequence having at least 95% identity to an amino acid sequence of SEQ ID NO:2651. In one aspect, provided herein is an antibody that binds TRDV2, comprising a light chain having an amino acid sequence having at least 95% identity to an amino acid sequence of SEQ ID NO:2652. In one aspect, provided herein is an antibody that binds TRDV2, comprising a heavy chain having an amino acid sequence having at least 95% identity to an amino acid sequence of SEQ ID NO:2651, and a light chain having an amino acid sequence having at least 95% identity to an amino acid sequence of SEQ ID NO:2652.

In one aspect, provided herein is an antibody that binds TRDV2, comprising: (i) a VH comprising a VH CDR1, a VH CDR2, and a VH CDR3 having an amino acid sequence of SEQ ID NOs:2653, 2654, and 2655, respectively, and (ii) a VL comprising a VL CDR1, VL CDR2, and VL CDR3 having an amino acid sequence of SEQ ID NOs:2656, 2657, and 2658, respectively. In one aspect, provided herein is an antibody that binds TRDV2, comprising: (i) a VH comprising a VH CDR1, a VH CDR2, and a VH CDR3 having an amino acid sequence of SEQ ID NOs:2659, 2660, and 2661, respectively, and (ii) a VL comprising a VL CDR1, VL CDR2, and VL CDR3 having an amino acid sequence of SEQ ID NOs:2662, 2663, and 2664, respectively. In one aspect, provided herein is an antibody that binds TRDV2, comprising: (i) a VH comprising a VH CDR1, a VH CDR2, and a VH CDR3 having an amino acid sequence of SEQ ID NOs:2665, 2666, and 2667, respectively, and (ii) a VL comprising a VL CDR1, VL CDR2, and VL CDR3 having an amino acid sequence of SEQ ID NOs:2668, 2669, and 2670, respectively. In one aspect, provided herein is an antibody that binds TRDV2, comprising: (i) a VH comprising a VH CDR1, a VH CDR2, and a VH CDR3 having an amino acid sequence of SEQ ID NOs:2671, 2672, and 2673, respectively, and (ii) a VL comprising a VL CDR1, VL CDR2, and VL CDR3 having an amino acid sequence of SEQ ID NOs:2674, 2675, and 2676, respectively. In one aspect, provided herein is an antibody that binds TRDV2, comprising: (i) a VH comprising a VH CDR1, a VH CDR2, and a VH CDR3 having an amino acid sequence of SEQ ID NOs:2677, 2678, and 2679, respectively, and (ii) a VL comprising a VL CDR1, VL CDR2, and VL CDR3 having an amino acid sequence of SEQ ID NOs:2680, 2681, and 2682, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2683; and (ii) a VL comprising a VL CDR1, a VL CDR2, and a VL CDR3 having an amino acid sequence of a VL CDR1, a VL CDR2, and a VL CDR3, respectively, of SEQ ID NO:2684. In one aspect, provided herein is an antibody that binds TRDV2, comprising a VH having an amino acid sequence of SEQ ID NO:2683. In one aspect, provided herein is an antibody that binds TRDV2, comprising a VL having an amino acid sequence of SEQ ID NO:2684. In one aspect, provided herein is an antibody that binds TRDV2, comprising a VH having an amino acid sequence of SEQ ID NO:2683, and a VL having an amino acid sequence of SEQ ID NO:2684. In one aspect, provided herein is an antibody that binds TRDV2, comprising a heavy chain having an amino acid sequence of SEQ ID NO:2685. In one aspect, provided herein is an antibody that binds TRDV2, comprising a light chain having an amino acid sequence of SEQ ID NO:2686. In one aspect, provided herein is an antibody that binds TRDV2, comprising a heavy chain having an amino acid sequence of SEQ ID NO:2685, and a light chain having an amino acid sequence of SEQ ID NO:2686. In one aspect, provided herein is an antibody that binds TRDV2, comprising a VH having an amino acid sequence having at least 95% identity to an amino acid sequence of SEQ ID NO:2683. In one aspect, provided herein is an antibody that binds TRDV2, comprising a VL having an amino acid sequence having at least 95% identity to an amino acid sequence of SEQ ID NO:2684. In one aspect, provided herein is an antibody that binds TRDV2, comprising a VH having an amino acid sequence having at least 95% identity to an amino acid sequence of SEQ ID NO:2683, and a VL having an amino acid sequence having at least 95% identity to an amino acid sequence of SEQ ID NO:2684. In one aspect, provided herein is an antibody that binds TRDV2, comprising a heavy chain having an amino acid sequence having at least 95% identity to an amino acid sequence of SEQ ID NO:2685. In one aspect, provided herein is an antibody that binds TRDV2, comprising a light chain having an amino acid sequence having at least 95% identity to an amino acid sequence of SEQ ID NO:2686. In one aspect, provided herein is an antibody that binds TRDV2, comprising a heavy chain having an amino acid sequence having at least 95% identity to an amino acid sequence of SEQ ID NO:2685, and a light chain having an amino acid sequence having at least 95% identity to an amino acid sequence of SEQ ID NO:266.

In one aspect, provided herein is an antibody that binds TRDV2, comprising: (i) a VH comprising a VH CDR1, a VH CDR2, and a VH CDR3 having an amino acid sequence of SEQ ID NOs:2687, 2688, and 2689, respectively, and (ii) a VL comprising a VL CDR1, VL CDR2, and VL CDR3 having an amino acid sequence of SEQ ID NOs:2690, 2691, and 2692, respectively. In one aspect, provided herein is an antibody that binds TRDV2, comprising: (i) a VH comprising a VH CDR1, a VH CDR2, and a VH CDR3 having an amino acid sequence of SEQ ID NOs:2693, 2694, and 2695, respectively, and (ii) a VL comprising a VL CDR1, VL CDR2, and VL CDR3 having an amino acid sequence of SEQ ID NOs:2696, 2697, and 2698, respectively. In one aspect, provided herein is an antibody that binds TRDV2, comprising: (i) a VH comprising a VH CDR1, a VH CDR2, and a VH CDR3 having an amino acid sequence of SEQ ID NOs:2699, 2700, and 2701, respectively, and (ii) a VL comprising a VL CDR1, VL CDR2, and VL CDR3 having an amino acid sequence of SEQ ID NOs:2702, 2703, and 2704, respectively. In one aspect, provided herein is an antibody that binds TRDV2, comprising: (i) a VH comprising a VH CDR1, a VH CDR2, and a VH CDR3 having an amino acid sequence of SEQ ID NOs:2705, 2706, and 2707, respectively, and (ii) a VL comprising a VL CDR1, VL CDR2, and VL CDR3 having an amino acid sequence of SEQ ID NOs:2708, 2709, and 2710, respectively. In one aspect, provided herein is an antibody that binds TRDV2, comprising: (i) a VH comprising a VH CDR1, a VH CDR2, and a VH CDR3 having an amino acid sequence of SEQ ID NOs:2711, 2712, and 2713, respectively, and (ii) a VL comprising a VL CDR1, VL CDR2, and VL CDR3 having an amino acid sequence of SEQ ID NOs:2714, 2715, and 2716, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2717; and (ii) a VL comprising a VL CDR1, a VL CDR2, and a VL CDR3 having an amino acid sequence of a VL CDR1, a VL CDR2, and a VL CDR3, respectively, of SEQ ID NO:2718. In one aspect, provided herein is an antibody that binds TRDV2, comprising a VH having an amino acid sequence of SEQ ID NO:2717. In one aspect, provided herein is an antibody that binds TRDV2, comprising a VL having an amino acid sequence of SEQ ID NO:2718. In one aspect, provided herein is an antibody that binds TRDV2, comprising a VH having an amino acid sequence of SEQ ID NO:2717, and a VL having an amino acid sequence of SEQ ID NO:2718. In one aspect, provided herein is an antibody that binds TRDV2, comprising a heavy chain having an amino acid sequence of SEQ ID NO:2719. In one aspect, provided herein is an antibody that binds TRDV2, comprising a light chain having an amino acid sequence of SEQ ID NO:2720. In one aspect, provided herein is an antibody that binds TRDV2, comprising a heavy chain having an amino acid sequence of SEQ ID NO:2719, and a light chain having an amino acid sequence of SEQ ID NO:2720. In one aspect, provided herein is an antibody that binds TRDV2, comprising a VH having an amino acid sequence having at least 95% identity to an amino acid sequence of SEQ ID NO:2717. In one aspect, provided herein is an antibody that binds TRDV2, comprising a VL having an amino acid sequence having at least 95% identity to an amino acid sequence of SEQ ID NO:2718. In one aspect, provided herein is an antibody that binds TRDV2, comprising a VH having an amino acid sequence having at least 95% identity to an amino acid sequence of SEQ ID NO:2717, and a VL having an amino acid sequence having at least 95% identity to an amino acid sequence of SEQ ID NO:2718. In one aspect, provided herein is an antibody that binds TRDV2, comprising a heavy chain having an amino acid sequence having at least 95% identity to an amino acid sequence of SEQ ID NO:2719. In one aspect, provided herein is an antibody that binds TRDV2, comprising a light chain having an amino acid sequence having at least 95% identity to an amino acid sequence of SEQ ID NO:2720. In one aspect, provided herein is an antibody that binds TRDV2, comprising a heavy chain having an amino acid sequence having at least 95% identity to an amino acid sequence of SEQ ID NO:2719, and a light chain having an amino acid sequence having at least 95% identity to an amino acid sequence of SEQ ID NO:2720.

In one aspect, provided herein is an antibody that binds TRDV2, comprising: (i) a VH comprising a VH CDR1, a VH CDR2, and a VH CDR3 having an amino acid sequence of SEQ ID NOs:2721, 2722, and 2723, respectively, and (ii) a VL comprising a VL CDR1, VL CDR2, and VL CDR3 having an amino acid sequence of SEQ ID NOs:2724, 2725, and 2726, respectively. In one aspect, provided herein is an antibody that binds TRDV2, comprising: (i) a VH comprising a VH CDR1, a VH CDR2, and a VH CDR3 having an amino acid sequence of SEQ ID NOs:2727, 2728, and 2729, respectively, and (ii) a VL comprising a VL CDR1, VL CDR2, and VL CDR3 having an amino acid sequence of SEQ ID NOs:2730, 2731, and 2732, respectively. In one aspect, provided herein is an antibody that binds TRDV2, comprising: (i) a VH comprising a VH CDR1, a VH CDR2, and a VH CDR3 having an amino acid sequence of SEQ ID NOs:2733, 2734, and 2735, respectively, and (ii) a VL comprising a VL CDR1, VL CDR2, and VL CDR3 having an amino acid sequence of SEQ ID NOs:2736, 2737, and 2738, respectively. In one aspect, provided herein is an antibody that binds TRDV2, comprising: (i) a VH comprising a VH CDR1, a VH CDR2, and a VH CDR3 having an amino acid sequence of SEQ ID NOs:2739, 2740, and 2741, respectively, and (ii) a VL comprising a VL CDR1, VL CDR2, and VL CDR3 having an amino acid sequence of SEQ ID NOs:2742, 2743, and 2744, respectively. In one aspect, provided herein is an antibody that binds TRDV2, comprising: (i) a VH comprising a VH CDR1, a VH CDR2, and a VH CDR3 having an amino acid sequence of SEQ ID NOs:2745, 2746, and 2747, respectively, and (ii) a VL comprising a VL CDR1, VL CDR2, and VL CDR3 having an amino acid sequence of SEQ ID NOs:2748, 2749, and 2750, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2751; and (ii) a VL comprising a VL CDR1, a VL CDR2, and a VL CDR3 having an amino acid sequence of a VL CDR1, a VL CDR2, and a VL CDR3, respectively, of SEQ ID NO:2752. In one aspect, provided herein is an antibody that binds TRDV2, comprising a VH having an amino acid sequence of SEQ ID NO:2751. In one aspect, provided herein is an antibody that binds TRDV2, comprising a VL having an amino acid sequence of SEQ ID NO:2752. In one aspect, provided herein is an antibody that binds TRDV2, comprising a VH having an amino acid sequence of SEQ ID NO:2751, and a VL having an amino acid sequence of SEQ ID NO:2752. In one aspect, provided herein is an antibody that binds TRDV2, comprising a heavy chain having an amino acid sequence of SEQ ID NO:2753. In one aspect, provided herein is an antibody that binds TRDV2, comprising a light chain having an amino acid sequence of SEQ ID NO:2754. In one aspect, provided herein is an antibody that binds TRDV2, comprising a heavy chain having an amino acid sequence of SEQ ID NO:2753, and a light chain having an amino acid sequence of SEQ ID NO:2754. In one aspect, provided herein is an antibody that binds TRDV2, comprising a VH having an amino acid sequence having at least 95% identity to an amino acid sequence of SEQ ID NO:2751. In one aspect, provided herein is an antibody that binds TRDV2, comprising a VL having an amino acid sequence having at least 95% identity to an amino acid sequence of SEQ ID NO:2752. In one aspect, provided herein is an antibody that binds TRDV2, comprising a VH having an amino acid sequence having at least 95% identity to an amino acid sequence of SEQ ID NO:2751, and a VL having an amino acid sequence having at least 95% identity to an amino acid sequence of SEQ ID NO:2752. In one aspect, provided herein is an antibody that binds TRDV2, comprising a heavy chain having an amino acid sequence having at least 95% identity to an amino acid sequence of SEQ ID NO:2753. In one aspect, provided herein is an antibody that binds TRDV2, comprising a light chain having an amino acid sequence having at least 95% identity to an amino acid sequence of SEQ ID NO:2754. In one aspect, provided herein is an antibody that binds TRDV2, comprising a heavy chain having an amino acid sequence having at least 95% identity to an amino acid sequence of SEQ ID NO:2753, and a light chain having an amino acid sequence having at least 95% identity to an amino acid sequence of SEQ ID NO:2754.

In one aspect, provided herein is an antibody that binds TRDV2, comprising: (i) a VH comprising a VH CDR1, a VH CDR2, and a VH CDR3 having an amino acid sequence of SEQ ID NOs:2755, 2756, and 2757, respectively, and (ii) a VL comprising a VL CDR1, VL CDR2, and VL CDR3 having an amino acid sequence of SEQ ID NOs:2758, 2759, and 2760, respectively. In one aspect, provided herein is an antibody that binds TRDV2, comprising: (i) a VH comprising a VH CDR1, a VH CDR2, and a VH CDR3 having an amino acid sequence of SEQ ID NOs:2761, 2762, and 2763, respectively, and (ii) a VL comprising a VL CDR1, VL CDR2, and VL CDR3 having an amino acid sequence of SEQ ID NOs:2764, 2765, and 2766, respectively. In one aspect, provided herein is an antibody that binds TRDV2, comprising: (i) a VH comprising a VH CDR1, a VH CDR2, and a VH CDR3 having an amino acid sequence of SEQ ID NOs:2767, 2768, and 2769, respectively, and (ii) a VL comprising a VL CDR1, VL CDR2, and VL CDR3 having an amino acid sequence of SEQ ID NOs:2770, 2771, and 2772, respectively. In one aspect, provided herein is an antibody that binds TRDV2, comprising: (i) a VH comprising a VH CDR1, a VH CDR2, and a VH CDR3 having an amino acid sequence of SEQ ID NOs:2773, 2774, and 2775, respectively, and (ii) a VL comprising a VL CDR1, VL CDR2, and VL CDR3 having an amino acid sequence of SEQ ID NOs:2776, 2777, and 2778, respectively. In one aspect, provided herein is an antibody that binds TRDV2, comprising: (i) a VH comprising a VH CDR1, a VH CDR2, and a VH CDR3 having an amino acid sequence of SEQ ID NOs:2779, 2780, and 2781, respectively, and (ii) a VL comprising a VL CDR1, VL CDR2, and VL CDR3 having an amino acid sequence of SEQ ID NOs:2782, 2783, and 2784, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2785; and (ii) a VL comprising a VL CDR1, a VL CDR2, and a VL CDR3 having an amino acid sequence of a VL CDR1, a VL CDR2, and a VL CDR3, respectively, of SEQ ID NO:2786. In one aspect, provided herein is an antibody that binds TRDV2, comprising a VH having an amino acid sequence of SEQ ID NO:2785. In one aspect, provided herein is an antibody that binds TRDV2, comprising a VL having an amino acid sequence of SEQ ID NO:2786. In one aspect, provided herein is an antibody that binds TRDV2, comprising a VH having an amino acid sequence of SEQ ID NO:2785, and a VL having an amino acid sequence of SEQ ID NO:2786. In one aspect, provided herein is an antibody that binds TRDV2, comprising a heavy chain having an amino acid sequence of SEQ ID NO:2787. In one aspect, provided herein is an antibody that binds TRDV2, comprising a light chain having an amino acid sequence of SEQ ID NO:2788. In one aspect, provided herein is an antibody that binds TRDV2, comprising a heavy chain having an amino acid sequence of SEQ ID NO:2787, and a light chain having an amino acid sequence of SEQ ID NO:2788. In one aspect, provided herein is an antibody that binds TRDV2, comprising a VH having an amino acid sequence having at least 95% identity to an amino acid sequence of SEQ ID NO:2785. In one aspect, provided herein is an antibody that binds TRDV2, comprising a VL having an amino acid sequence having at least 95% identity to an amino acid sequence of SEQ ID NO:2786. In one aspect, provided herein is an antibody that binds TRDV2, comprising a VH having an amino acid sequence having at least 95% identity to an amino acid sequence of SEQ ID NO:2785, and a VL having an amino acid sequence having at least 95% identity to an amino acid sequence of SEQ ID NO:2786. In one aspect, provided herein is an antibody that binds TRDV2, comprising a heavy chain having an amino acid sequence having at least 95% identity to an amino acid sequence of SEQ ID NO:2787. In one aspect, provided herein is an antibody that binds TRDV2, comprising a light chain having an amino acid sequence having at least 95% identity to an amino acid sequence of SEQ ID NO:2788. In one aspect, provided herein is an antibody that binds TRDV2, comprising a heavy chain having an amino acid sequence having at least 95% identity to an amino acid sequence of SEQ ID NO:2787, and a light chain having an amino acid sequence having at least 95% identity to an amino acid sequence of SEQ ID NO:2788.

In one aspect, provided herein is an antibody that binds TRDV2, comprising: (i) a VH comprising a VH CDR1, a VH CDR2, and a VH CDR3 having an amino acid sequence of SEQ ID NOs:2789, 2790, and 2791, respectively, and (ii) a VL comprising a VL CDR1, VL CDR2, and VL CDR3 having an amino acid sequence of SEQ ID NOs:2792, 2793, and 2794, respectively. In one aspect, provided herein is an antibody that binds TRDV2, comprising: (i) a VH comprising a VH CDR1, a VH CDR2, and a VH CDR3 having an amino acid sequence of SEQ ID NOs:2795, 2796, and 2797, respectively, and (ii) a VL comprising a VL CDR1, VL CDR2, and VL CDR3 having an amino acid sequence of SEQ ID NOs:2798, 2799, and 2800, respectively. In one aspect, provided herein is an antibody that binds TRDV2, comprising: (i) a VH comprising a VH CDR1, a VH CDR2, and a VH CDR3 having an amino acid sequence of SEQ ID NOs:2801, 2802, and 2803, respectively, and (ii) a VL comprising a VL CDR1, VL CDR2, and VL CDR3 having an amino acid sequence of SEQ ID NOs:2804, 2805, and 2806, respectively. In one aspect, provided herein is an antibody that binds TRDV2, comprising: (i) a VH comprising a VH CDR1, a VH CDR2, and a VH CDR3 having an amino acid sequence of SEQ ID NOs:2807, 2808, and 2809, respectively, and (ii) a VL comprising a VL CDR1, VL CDR2, and VL CDR3 having an amino acid sequence of SEQ ID NOs:2810, 2811, and 2812, respectively. In one aspect, provided herein is an antibody that binds TRDV2, comprising: (i) a VH comprising a VH CDR1, a VH CDR2, and a VH CDR3 having an amino acid sequence of SEQ ID NOs:2813, 2814, and 2815, respectively, and (ii) a VL comprising a VL CDR1, VL CDR2, and VL CDR3 having an amino acid sequence of SEQ ID NOs:2816, 2817, and 2818, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2819; and (ii) a VL comprising a VL CDR1, a VL CDR2, and a VL CDR3 having an amino acid sequence of a VL CDR1, a VL CDR2, and a VL CDR3, respectively, of SEQ ID NO:2820. In one aspect, provided herein is an antibody that binds TRDV2, comprising a VH having an amino acid sequence of SEQ ID NO:2819. In one aspect, provided herein is an antibody that binds TRDV2, comprising a VL having an amino acid sequence of SEQ ID NO:2820. In one aspect, provided herein is an antibody that binds TRDV2, comprising a VH having an amino acid sequence of SEQ ID NO:2819, and a VL having an amino acid sequence of SEQ ID NO:2820. In one aspect, provided herein is an antibody that binds TRDV2, comprising a heavy chain having an amino acid sequence of SEQ ID NO:2821. In one aspect, provided herein is an antibody that binds TRDV2, comprising a light chain having an amino acid sequence of SEQ ID NO:2822. In one aspect, provided herein is an antibody that binds TRDV2, comprising a heavy chain having an amino acid sequence of SEQ ID NO:2821, and a light chain having an amino acid sequence of SEQ ID NO:2822. In one aspect, provided herein is an antibody that binds TRDV2, comprising a VH having an amino acid sequence having at least 95% identity to an amino acid sequence of SEQ ID NO:2819. In one aspect, provided herein is an antibody that binds TRDV2, comprising a VL having an amino acid sequence having at least 95% identity to an amino acid sequence of SEQ ID NO:2820. In one aspect, provided herein is an antibody that binds TRDV2, comprising a VH having an amino acid sequence having at least 95% identity to an amino acid sequence of SEQ ID NO:2819, and a VL having an amino acid sequence having at least 95% identity to an amino acid sequence of SEQ ID NO:2820. In one aspect, provided herein is an antibody that binds TRDV2, comprising a heavy chain having an amino acid sequence having at least 95% identity to an amino acid sequence of SEQ ID NO:2821. In one aspect, provided herein is an antibody that binds TRDV2, comprising a light chain having an amino acid sequence having at least 95% identity to an amino acid sequence of SEQ ID NO:2822. In one aspect, provided herein is an antibody that binds TRDV2, comprising a heavy chain having an amino acid sequence having at least 95% identity to an amino acid sequence of SEQ ID NO:2821, and a light chain having an amino acid sequence having at least 95% identity to an amino acid sequence of SEQ ID NO:2822.

In one aspect, provided herein is an antibody that binds TRDV2, comprising: (i) a VH comprising a VH CDR1, a VH CDR2, and a VH CDR3 having an amino acid sequence of SEQ ID NOs:2823, 2824, and 2825, respectively, and (ii) a VL comprising a VL CDR1, VL CDR2, and VL CDR3 having an amino acid sequence of SEQ ID NOs:2826, 2827, and 2828, respectively. In one aspect, provided herein is an antibody that binds TRDV2, comprising: (i) a VH comprising a VH CDR1, a VH CDR2, and a VH CDR3 having an amino acid sequence of SEQ ID NOs:2829, 2830, and 2831, respectively, and (ii) a VL comprising a VL CDR1, VL CDR2, and VL CDR3 having an amino acid sequence of SEQ ID NOs:2832, 2833, and 2834, respectively. In one aspect, provided herein is an antibody that binds TRDV2, comprising: (i) a VH comprising a VH CDR1, a VH CDR2, and a VH CDR3 having an amino acid sequence of SEQ ID NOs:2835, 2836, and 2837, respectively, and (ii) a VL comprising a VL CDR1, VL CDR2, and VL CDR3 having an amino acid sequence of SEQ ID NOs:2838, 2839, and 2840, respectively. In one aspect, provided herein is an antibody that binds TRDV2, comprising: (i) a VH comprising a VH CDR1, a VH CDR2, and a VH CDR3 having an amino acid sequence of SEQ ID NOs:2841, 2842, and 2843, respectively, and (ii) a VL comprising a VL CDR1, VL CDR2, and VL CDR3 having an amino acid sequence of SEQ ID NOs:2844, 2845, and 2846, respectively. In one aspect, provided herein is an antibody that binds TRDV2, comprising: (i) a VH comprising a VH CDR1, a VH CDR2, and a VH CDR3 having an amino acid sequence of SEQ ID NOs:2847, 2848, and 2849, respectively, and (ii) a VL comprising a VL CDR1, VL CDR2, and VL CDR3 having an amino acid sequence of SEQ ID NOs:2850, 2851, and 2852, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2853; and (ii) a VL comprising a VL CDR1, a VL CDR2, and a VL CDR3 having an amino acid sequence of a VL CDR1, a VL CDR2, and a VL CDR3, respectively, of SEQ ID NO:2854. In one aspect, provided herein is an antibody that binds TRDV2, comprising a VH having an amino acid sequence of SEQ ID NO:2853. In one aspect, provided herein is an antibody that binds TRDV2, comprising a VL having an amino acid sequence of SEQ ID NO:2854. In one aspect, provided herein is an antibody that binds TRDV2, comprising a VH having an amino acid sequence of SEQ ID NO:2853, and a VL having an amino acid sequence of SEQ ID NO:2854. In one aspect, provided herein is an antibody that binds TRDV2, comprising a heavy chain having an amino acid sequence of SEQ ID NO:2855. In one aspect, provided herein is an antibody that binds TRDV2, comprising a light chain having an amino acid sequence of SEQ ID NO:2856. In one aspect, provided herein is an antibody that binds TRDV2, comprising a heavy chain having an amino acid sequence of SEQ ID NO:2855, and a light chain having an amino acid sequence of SEQ ID NO:2856. In one aspect, provided herein is an antibody that binds TRDV2, comprising a VH having an amino acid sequence having at least 95% identity to an amino acid sequence of SEQ ID NO:2853. In one aspect, provided herein is an antibody that binds TRDV2, comprising a VL having an amino acid sequence having at least 95% identity to an amino acid sequence of SEQ ID NO:2854. In one aspect, provided herein is an antibody that binds TRDV2, comprising a VH having an amino acid sequence having at least 95% identity to an amino acid sequence of SEQ ID NO:2853, and a VL having an amino acid sequence having at least 95% identity to an amino acid sequence of SEQ ID NO:2854. In one aspect, provided herein is an antibody that binds TRDV2, comprising a heavy chain having an amino acid sequence having at least 95% identity to an amino acid sequence of SEQ ID NO:2855. In one aspect, provided herein is an antibody that binds TRDV2, comprising a light chain having an amino acid sequence having at least 95% identity to an amino acid sequence of SEQ ID NO:2856. In one aspect, provided herein is an antibody that binds TRDV2, comprising a heavy chain having an amino acid sequence having at least 95% identity to an amino acid sequence of SEQ ID NO:2855, and a light chain having an amino acid sequence having at least 95% identity to an amino acid sequence of SEQ ID NO:2856.

In one aspect, provided herein is an antibody that binds TRDV2, comprising: (i) a VH comprising a VH CDR1, a VH CDR2, and a VH CDR3 having an amino acid sequence of SEQ ID NOs:2857, 2858, and 2859, respectively, and (ii) a VL comprising a VL CDR1, VL CDR2, and VL CDR3 having an amino acid sequence of SEQ ID NOs:2860, 2861, and 2862, respectively. In one aspect, provided herein is an antibody that binds TRDV2, comprising: (i) a VH comprising a VH CDR1, a VH CDR2, and a VH CDR3 having an amino acid sequence of SEQ ID NOs:2863, 2864, and 2865, respectively, and (ii) a VL comprising a VL CDR1, VL CDR2, and VL CDR3 having an amino acid sequence of SEQ ID NOs:2866, 2867, and 2868, respectively. In one aspect, provided herein is an antibody that binds TRDV2, comprising: (i) a VH comprising a VH CDR1, a VH CDR2, and a VH CDR3 having an amino acid sequence of SEQ ID NOs:2869, 2870, and 2871, respectively, and (ii) a VL comprising a VL CDR1, VL CDR2, and VL CDR3 having an amino acid sequence of SEQ ID NOs:2872, 2873, and 2874, respectively. In one aspect, provided herein is an antibody that binds TRDV2, comprising: (i) a VH comprising a VH CDR1, a VH CDR2, and a VH CDR3 having an amino acid sequence of SEQ ID NOs:2875, 2876, and 2877, respectively, and (ii) a VL comprising a VL CDR1, VL CDR2, and VL CDR3 having an amino acid sequence of SEQ ID NOs:2878, 2879, and 2880, respectively. In one aspect, provided herein is an antibody that binds TRDV2, comprising: (i) a VH comprising a VH CDR1, a VH CDR2, and a VH CDR3 having an amino acid sequence of SEQ ID NOs:2881, 2882, and 2883, respectively, and (ii) a VL comprising a VL CDR1, VL CDR2, and VL CDR3 having an amino acid sequence of SEQ ID NOs:2884, 2885, and 2886, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2887; and (ii) a VL comprising a VL CDR1, a VL CDR2, and a VL CDR3 having an amino acid sequence of a VL CDR1, a VL CDR2, and a VL CDR3, respectively, of SEQ ID NO:2888. In one aspect, provided herein is an antibody that binds TRDV2, comprising a VH having an amino acid sequence of SEQ ID NO:2887. In one aspect, provided herein is an antibody that binds TRDV2, comprising a VL having an amino acid sequence of SEQ ID NO:2888. In one aspect, provided herein is an antibody that binds TRDV2, comprising a VH having an amino acid sequence of SEQ ID NO:2887, and a VL having an amino acid sequence of SEQ ID NO:2888. In one aspect, provided herein is an antibody that binds TRDV2, comprising a heavy chain having an amino acid sequence of SEQ ID NO:2889. In one aspect, provided herein is an antibody that binds TRDV2, comprising a light chain having an amino acid sequence of SEQ ID NO:2890. In one aspect, provided herein is an antibody that binds TRDV2, comprising a heavy chain having an amino acid sequence of SEQ ID NO:2889, and a light chain having an amino acid sequence of SEQ ID NO:2890. In one aspect, provided herein is an antibody that binds TRDV2, comprising a VH having an amino acid sequence having at least 95% identity to an amino acid sequence of SEQ ID NO:2887. In one aspect, provided herein is an antibody that binds TRDV2, comprising a VL having an amino acid sequence having at least 95% identity to an amino acid sequence of SEQ ID NO:2888. In one aspect, provided herein is an antibody that binds TRDV2, comprising a VH having an amino acid sequence having at least 95% identity to an amino acid sequence of SEQ ID NO:2887, and a VL having an amino acid sequence having at least 95% identity to an amino acid sequence of SEQ ID NO:2888. In one aspect, provided herein is an antibody that binds TRDV2, comprising a heavy chain having an amino acid sequence having at least 95% identity to an amino acid sequence of SEQ ID NO:2889. In one aspect, provided herein is an antibody that binds TRDV2, comprising a light chain having an amino acid sequence having at least 95% identity to an amino acid sequence of SEQ ID NO:2890. In one aspect, provided herein is an antibody that binds TRDV2, comprising a heavy chain having an amino acid sequence having at least 95% identity to an amino acid sequence of SEQ ID NO:2889, and a light chain having an amino acid sequence having at least 95% identity to an amino acid sequence of SEQ ID NO:2890.

In one aspect, provided herein is an antibody that binds TRDV2, comprising: (i) a VH comprising a VH CDR1, a VH CDR2, and a VH CDR3 having an amino acid sequence of SEQ ID NOs:2891, 2892, and 2893, respectively, and (ii) a VL comprising a VL CDR1, VL CDR2, and VL CDR3 having an amino acid sequence of SEQ ID NOs:2894, 2895, and 2896, respectively. In one aspect, provided herein is an antibody that binds TRDV2, comprising: (i) a VH comprising a VH CDR1, a VH CDR2, and a VH CDR3 having an amino acid sequence of SEQ ID NOs:2897, 2898, and 2899, respectively, and (ii) a VL comprising a VL CDR1, VL CDR2, and VL CDR3 having an amino acid sequence of SEQ ID NOs:2900, 2901, and 2902, respectively. In one aspect, provided herein is an antibody that binds TRDV2, comprising: (i) a VH comprising a VH CDR1, a VH CDR2, and a VH CDR3 having an amino acid sequence of SEQ ID NOs:2903, 2904, and 2905, respectively, and (ii) a VL comprising a VL CDR1, VL CDR2, and VL CDR3 having an amino acid sequence of SEQ ID NOs:2906, 2907, and 2908, respectively. In one aspect, provided herein is an antibody that binds TRDV2, comprising: (i) a VH comprising a VH CDR1, a VH CDR2, and a VH CDR3 having an amino acid sequence of SEQ ID NOs:2909, 2910, and 2911, respectively, and (ii) a VL comprising a VL CDR1, VL CDR2, and VL CDR3 having an amino acid sequence of SEQ ID NOs:2912, 2913, and 2914, respectively. In one aspect, provided herein is an antibody that binds TRDV2, comprising: (i) a VH comprising a VH CDR1, a VH CDR2, and a VH CDR3 having an amino acid sequence of SEQ ID NOs:2915, 2916, and 2917, respectively, and (ii) a VL comprising a VL CDR1, VL CDR2, and VL CDR3 having an amino acid sequence of SEQ ID NOs:2918, 2919, and 2920, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2921; and (ii) a VL comprising a VL CDR1, a VL CDR2, and a VL CDR3 having an amino acid sequence of a VL CDR1, a VL CDR2, and a VL CDR3, respectively, of SEQ ID NO:2922. In one aspect, provided herein is an antibody that binds TRDV2, comprising a VH having an amino acid sequence of SEQ ID NO:2921. In one aspect, provided herein is an antibody that binds TRDV2, comprising a VL having an amino acid sequence of SEQ ID NO:2922. In one aspect, provided herein is an antibody that binds TRDV2, comprising a VH having an amino acid sequence of SEQ ID NO:2921, and a VL having an amino acid sequence of SEQ ID NO:2922. In one aspect, provided herein is an antibody that binds TRDV2, comprising a heavy chain having an amino acid sequence of SEQ ID NO:2923. In one aspect, provided herein is an antibody that binds TRDV2, comprising a light chain having an amino acid sequence of SEQ ID NO:2924. In one aspect, provided herein is an antibody that binds TRDV2, comprising a heavy chain having an amino acid sequence of SEQ ID NO:2923, and a light chain having an amino acid sequence of SEQ ID NO:2924. In one aspect, provided herein is an antibody that binds TRDV2, comprising a VH having an amino acid sequence having at least 95% identity to an amino acid sequence of SEQ ID NO:2921. In one aspect, provided herein is an antibody that binds TRDV2, comprising a VL having an amino acid sequence having at least 95% identity to an amino acid sequence of SEQ ID NO:2922. In one aspect, provided herein is an antibody that binds TRDV2, comprising a VH having an amino acid sequence having at least 95% identity to an amino acid sequence of SEQ ID NO:2921, and a VL having an amino acid sequence having at least 95% identity to an amino acid sequence of SEQ ID NO:2922. In one aspect, provided herein is an antibody that binds TRDV2, comprising a heavy chain having an amino acid sequence having at least 95% identity to an amino acid sequence of SEQ ID NO:2923. In one aspect, provided herein is an antibody that binds TRDV2, comprising a light chain having an amino acid sequence having at least 95% identity to an amino acid sequence of SEQ ID NO:2924. In one aspect, provided herein is an antibody that binds TRDV2, comprising a heavy chain having an amino acid sequence having at least 95% identity to an amino acid sequence of SEQ ID NO:2923, and a light chain having an amino acid sequence having at least 95% identity to an amino acid sequence of SEQ ID NO:2924.

In one aspect, provided herein is an antibody that binds TRDV2, comprising: (i) a VH comprising a VH CDR1, a VH CDR2, and a VH CDR3 having an amino acid sequence of SEQ ID NOs:2925, 2926, and 2927, respectively, and (ii) a VL comprising a VL CDR1, VL CDR2, and VL CDR3 having an amino acid sequence of SEQ ID NOs:2928, 2929, and 2930, respectively. In one aspect, provided herein is an antibody that binds TRDV2, comprising: (i) a VH comprising a VH CDR1, a VH CDR2, and a VH CDR3 having an amino acid sequence of SEQ ID NOs:2931, 2932, and 2933, respectively, and (ii) a VL comprising a VL CDR1, VL CDR2, and VL CDR3 having an amino acid sequence of SEQ ID NOs:2934, 2935, and 2936, respectively. In one aspect, provided herein is an antibody that binds TRDV2, comprising: (i) a VH comprising a VH CDR1, a VH CDR2, and a VH CDR3 having an amino acid sequence of SEQ ID NOs:2937, 2938, and 2939, respectively, and (ii) a VL comprising a VL CDR1, VL CDR2, and VL CDR3 having an amino acid sequence of SEQ ID NOs:2940, 2941, and 2942, respectively. In one aspect, provided herein is an antibody that binds TRDV2, comprising: (i) a VH comprising a VH CDR1, a VH CDR2, and a VH CDR3 having an amino acid sequence of SEQ ID NOs:2943, 2944, and 2945, respectively, and (ii) a VL comprising a VL CDR1, VL CDR2, and VL CDR3 having an amino acid sequence of SEQ ID NOs:2946, 2947, and 2948, respectively. In one aspect, provided herein is an antibody that binds TRDV2, comprising: (i) a VH comprising a VH CDR1, a VH CDR2, and a VH CDR3 having an amino acid sequence of SEQ ID NOs:2949, 2950, and 2951, respectively, and (ii) a VL comprising a VL CDR1, VL CDR2, and VL CDR3 having an amino acid sequence of SEQ ID NOs:2952, 2953, and 2954, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2955; and (ii) a VL comprising a VL CDR1, a VL CDR2, and a VL CDR3 having an amino acid sequence of a VL CDR1, a VL CDR2, and a VL CDR3, respectively, of SEQ ID NO:2956. In one aspect, provided herein is an antibody that binds TRDV2, comprising a VH having an amino acid sequence of SEQ ID NO:2955. In one aspect, provided herein is an antibody that binds TRDV2, comprising a VL having an amino acid sequence of SEQ ID NO:2956. In one aspect, provided herein is an antibody that binds TRDV2, comprising a VH having an amino acid sequence of SEQ ID NO:2955, and a VL having an amino acid sequence of SEQ ID NO:2956. In one aspect, provided herein is an antibody that binds TRDV2, comprising a heavy chain having an amino acid sequence of SEQ ID NO:2957. In one aspect, provided herein is an antibody that binds TRDV2, comprising a light chain having an amino acid sequence of SEQ ID NO:2958. In one aspect, provided herein is an antibody that binds TRDV2, comprising a heavy chain having an amino acid sequence of SEQ ID NO:2957, and a light chain having an amino acid sequence of SEQ ID NO:2958. In one aspect, provided herein is an antibody that binds TRDV2, comprising a VH having an amino acid sequence having at least 95% identity to an amino acid sequence of SEQ ID NO:2955. In one aspect, provided herein is an antibody that binds TRDV2, comprising a VL having an amino acid sequence having at least 95% identity to an amino acid sequence of SEQ ID NO:2956. In one aspect, provided herein is an antibody that binds TRDV2, comprising a VH having an amino acid sequence having at least 95% identity to an amino acid sequence of SEQ ID NO:2955, and a VL having an amino acid sequence having at least 95% identity to an amino acid sequence of SEQ ID NO:2956. In one aspect, provided herein is an antibody that binds TRDV2, comprising a heavy chain having an amino acid sequence having at least 95% identity to an amino acid sequence of SEQ ID NO:2957. In one aspect, provided herein is an antibody that binds TRDV2, comprising a light chain having an amino acid sequence having at least 95% identity to an amino acid sequence of SEQ ID NO:2958. In one aspect, provided herein is an antibody that binds TRDV2, comprising a heavy chain having an amino acid sequence having at least 95% identity to an amino acid sequence of SEQ ID NO:2957, and a light chain having an amino acid sequence having at least 95% identity to an amino acid sequence of SEQ ID NO:2958.

In one aspect, provided herein is an antibody that binds TRDV2, comprising: (i) a VH comprising a VH CDR1, a VH CDR2, and a VH CDR3 having an amino acid sequence of SEQ ID NOs:2959, 2960, and 2961, respectively, and (ii) a VL comprising a VL CDR1, VL CDR2, and VL CDR3 having an amino acid sequence of SEQ ID NOs:2962, 2963, and 2964, respectively. In one aspect, provided herein is an antibody that binds TRDV2, comprising: (i) a VH comprising a VH CDR1, a VH CDR2, and a VH CDR3 having an amino acid sequence of SEQ ID NOs:2965, 2966, and 2967, respectively, and (ii) a VL comprising a VL CDR1, VL CDR2, and VL CDR3 having an amino acid sequence of SEQ ID NOs:2968, 2969, and 2970, respectively. In one aspect, provided herein is an antibody that binds TRDV2, comprising: (i) a VH comprising a VH CDR1, a VH CDR2, and a VH CDR3 having an amino acid sequence of SEQ ID NOs:2971, 2972, and 2973, respectively, and (ii) a VL comprising a VL CDR1, VL CDR2, and VL CDR3 having an amino acid sequence of SEQ ID NOs:2974, 2975, and 2976, respectively. In one aspect, provided herein is an antibody that binds TRDV2, comprising: (i) a VH comprising a VH CDR1, a VH CDR2, and a VH CDR3 having an amino acid sequence of SEQ ID NOs:2977, 2978, and 2979, respectively, and (ii) a VL comprising a VL CDR1, VL CDR2, and VL CDR3 having an amino acid sequence of SEQ ID NOs:2980, 2981, and 2982, respectively. In one aspect, provided herein is an antibody that binds TRDV2, comprising: (i) a VH comprising a VH CDR1, a VH CDR2, and a VH CDR3 having an amino acid sequence of SEQ ID NOs:2983, 2984, and 2985, respectively, and (ii) a VL comprising a VL CDR1, VL CDR2, and VL CDR3 having an amino acid sequence of SEQ ID NOs:2986, 2987, and 2988, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2989; and (ii) a VL comprising a VL CDR1, a VL CDR2, and a VL CDR3 having an amino acid sequence of a VL CDR1, a VL CDR2, and a VL CDR3, respectively, of SEQ ID NO:2990. In one aspect, provided herein is an antibody that binds TRDV2, comprising a VH having an amino acid sequence of SEQ ID NO:2989. In one aspect, provided herein is an antibody that binds TRDV2, comprising a VL having an amino acid sequence of SEQ ID NO:2990. In one aspect, provided herein is an antibody that binds TRDV2, comprising a VH having an amino acid sequence of SEQ ID NO:2989, and a VL having an amino acid sequence of SEQ ID NO:2990. In one aspect, provided herein is an antibody that binds TRDV2, comprising a heavy chain having an amino acid sequence of SEQ ID NO:2991. In one aspect, provided herein is an antibody that binds TRDV2, comprising a light chain having an amino acid sequence of SEQ ID NO:2992. In one aspect, provided herein is an antibody that binds TRDV2, comprising a heavy chain having an amino acid sequence of SEQ ID NO:2991, and a light chain having an amino acid sequence of SEQ ID NO:2992. In one aspect, provided herein is an antibody that binds TRDV2, comprising a VH having an amino acid sequence having at least 95% identity to an amino acid sequence of SEQ ID NO:2989. In one aspect, provided herein is an antibody that binds TRDV2, comprising a VL having an amino acid sequence having at least 95% identity to an amino acid sequence of SEQ ID NO:2990. In one aspect, provided herein is an antibody that binds TRDV2, comprising a VH having an amino acid sequence having at least 95% identity to an amino acid sequence of SEQ ID NO:2989, and a VL having an amino acid sequence having at least 95% identity to an amino acid sequence of SEQ ID NO:2990. In one aspect, provided herein is an antibody that binds TRDV2, comprising a heavy chain having an amino acid sequence having at least 95% identity to an amino acid sequence of SEQ ID NO:2991. In one aspect, provided herein is an antibody that binds TRDV2, comprising a light chain having an amino acid sequence having at least 95% identity to an amino acid sequence of SEQ ID NO:2992. In one aspect, provided herein is an antibody that binds TRDV2, comprising a heavy chain having an amino acid sequence having at least 95% identity to an amino acid sequence of SEQ ID NO:2991, and a light chain having an amino acid sequence having at least 95% identity to an amino acid sequence of SEQ ID NO:2992.

In one aspect, provided herein is an antibody that binds TRDV2, comprising: (i) a VH comprising a VH CDR1, a VH CDR2, and a VH CDR3 having an amino acid sequence of SEQ ID NOs:2993, 2994, and 2995, respectively, and (ii) a VL comprising a VL CDR1, VL CDR2, and VL CDR3 having an amino acid sequence of SEQ ID NOs:2996, 2997, and 2998, respectively. In one aspect, provided herein is an antibody that binds TRDV2, comprising: (i) a VH comprising a VH CDR1, a VH CDR2, and a VH CDR3 having an amino acid sequence of SEQ ID NOs:2999, 3000, and 3001, respectively, and (ii) a VL comprising a VL CDR1, VL CDR2, and VL CDR3 having an amino acid sequence of SEQ ID NOs:3002, 3003, and 3004, respectively. In one aspect, provided herein is an antibody that binds TRDV2, comprising: (i) a VH comprising a VH CDR1, a VH CDR2, and a VH CDR3 having an amino acid sequence of SEQ ID NOs:3005, 3006, and 3007, respectively, and (ii) a VL comprising a VL CDR1, VL CDR2, and VL CDR3 having an amino acid sequence of SEQ ID NOs:3008, 3009, and 3010, respectively. In one aspect, provided herein is an antibody that binds TRDV2, comprising: (i) a VH comprising a VH CDR1, a VH CDR2, and a VH CDR3 having an amino acid sequence of SEQ ID NOs:3011, 3012, and 3013, respectively, and (ii) a VL comprising a VL CDR1, VL CDR2, and VL CDR3 having an amino acid sequence of SEQ ID NOs:3014, 3015, and 3016, respectively. In one aspect, provided herein is an antibody that binds TRDV2, comprising: (i) a VH comprising a VH CDR1, a VH CDR2, and a VH CDR3 having an amino acid sequence of SEQ ID NOs:3017, 3018, and 3019, respectively, and (ii) a VL comprising a VL CDR1, VL CDR2, and VL CDR3 having an amino acid sequence of SEQ ID NOs:3020, 3021, and 3022, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3023; and (ii) a VL comprising a VL CDR1, a VL CDR2, and a VL CDR3 having an amino acid sequence of a VL CDR1, a VL CDR2, and a VL CDR3, respectively, of SEQ ID NO:3024. In one aspect, provided herein is an antibody that binds TRDV2, comprising a VH having an amino acid sequence of SEQ ID NO:3023. In one aspect, provided herein is an antibody that binds TRDV2, comprising a VL having an amino acid sequence of SEQ ID NO:3024. In one aspect, provided herein is an antibody that binds TRDV2, comprising a VH having an amino acid sequence of SEQ ID NO:3023, and a VL having an amino acid sequence of SEQ ID NO:3024. In one aspect, provided herein is an antibody that binds TRDV2, comprising a heavy chain having an amino acid sequence of SEQ ID NO:3025. In one aspect, provided herein is an antibody that binds TRDV2, comprising a light chain having an amino acid sequence of SEQ ID NO:3026. In one aspect, provided herein is an antibody that binds TRDV2, comprising a heavy chain having an amino acid sequence of SEQ ID NO:3025, and a light chain having an amino acid sequence of SEQ ID NO:3026. In one aspect, provided herein is an antibody that binds TRDV2, comprising a VH having an amino acid sequence having at least 95% identity to an amino acid sequence of SEQ ID NO:3023. In one aspect, provided herein is an antibody that binds TRDV2, comprising a VL having an amino acid sequence having at least 95% identity to an amino acid sequence of SEQ ID NO:3024. In one aspect, provided herein is an antibody that binds TRDV2, comprising a VH having an amino acid sequence having at least 95% identity to an amino acid sequence of SEQ ID NO:3023, and a VL having an amino acid sequence having at least 95% identity to an amino acid sequence of SEQ ID NO:3024. In one aspect, provided herein is an antibody that binds TRDV2, comprising a heavy chain having an amino acid sequence having at least 95% identity to an amino acid sequence of SEQ ID NO:3025. In one aspect, provided herein is an antibody that binds TRDV2, comprising a light chain having an amino acid sequence having at least 95% identity to an amino acid sequence of SEQ ID NO:3026. In one aspect, provided herein is an antibody that binds TRDV2, comprising a heavy chain having an amino acid sequence having at least 95% identity to an amino acid sequence of SEQ ID NO:3025, and a light chain having an amino acid sequence having at least 95% identity to an amino acid sequence of SEQ ID NO:3026.

In one aspect, provided herein is an antibody that binds TRDV2, comprising: (i) a VH comprising a VH CDR1, a VH CDR2, and a VH CDR3 having an amino acid sequence of SEQ ID NOs:3027, 3028, and 3029, respectively, and (ii) a VL comprising a VL CDR1, VL CDR2, and VL CDR3 having an amino acid sequence of SEQ ID NOs:3030, 3031, and 3032, respectively. In one aspect, provided herein is an antibody that binds TRDV2, comprising: (i) a VH comprising a VH CDR1, a VH CDR2, and a VH CDR3 having an amino acid sequence of SEQ ID NOs:3033, 3034, and 3035, respectively, and (ii) a VL comprising a VL CDR1, VL CDR2, and VL CDR3 having an amino acid sequence of SEQ ID NOs:3036, 3037, and 3038, respectively. In one aspect, provided herein is an antibody that binds TRDV2, comprising: (i) a VH comprising a VH CDR1, a VH CDR2, and a VH CDR3 having an amino acid sequence of SEQ ID NOs:3039, 3040, and 3041, respectively, and (ii) a VL comprising a VL CDR1, VL CDR2, and VL CDR3 having an amino acid sequence of SEQ ID NOs:3042, 3043, and 3044, respectively. In one aspect, provided herein is an antibody that binds TRDV2, comprising: (i) a VH comprising a VH CDR1, a VH CDR2, and a VH CDR3 having an amino acid sequence of SEQ ID NOs:3045, 3046, and 3047, respectively, and (ii) a VL comprising a VL CDR1, VL CDR2, and VL CDR3 having an amino acid sequence of SEQ ID NOs:3048, 3049, and 3050, respectively. In one aspect, provided herein is an antibody that binds TRDV2, comprising: (i) a VH comprising a VH CDR1, a VH CDR2, and a VH CDR3 having an amino acid sequence of SEQ ID NOs:3051, 3052, and 3053, respectively, and (ii) a VL comprising a VL CDR1, VL CDR2, and VL CDR3 having an amino acid sequence of SEQ ID NOs:3054, 3055, and 3056, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3057; and (ii) a VL comprising a VL CDR1, a VL CDR2, and a VL CDR3 having an amino acid sequence of a VL CDR1, a VL CDR2, and a VL CDR3, respectively, of SEQ ID NO:3058. In one aspect, provided herein is an antibody that binds TRDV2, comprising a VH having an amino acid sequence of SEQ ID NO:3057. In one aspect, provided herein is an antibody that binds TRDV2, comprising a VL having an amino acid sequence of SEQ ID NO:3058. In one aspect, provided herein is an antibody that binds TRDV2, comprising a VH having an amino acid sequence of SEQ ID NO:3057, and a VL having an amino acid sequence of SEQ ID NO:3058. In one aspect, provided herein is an antibody that binds TRDV2, comprising a heavy chain having an amino acid sequence of SEQ ID NO:3059. In one aspect, provided herein is an antibody that binds TRDV2, comprising a light chain having an amino acid sequence of SEQ ID NO:3060. In one aspect, provided herein is an antibody that binds TRDV2, comprising a heavy chain having an amino acid sequence of SEQ ID NO:3059, and a light chain having an amino acid sequence of SEQ ID NO:3060. In one aspect, provided herein is an antibody that binds TRDV2, comprising a VH having an amino acid sequence having at least 95% identity to an amino acid sequence of SEQ ID NO:3057. In one aspect, provided herein is an antibody that binds TRDV2, comprising a VL having an amino acid sequence having at least 95% identity to an amino acid sequence of SEQ ID NO:3058. In one aspect, provided herein is an antibody that binds TRDV2, comprising a VH having an amino acid sequence having at least 95% identity to an amino acid sequence of SEQ ID NO:3057, and a VL having an amino acid sequence having at least 95% identity to an amino acid sequence of SEQ ID NO:3058. In one aspect, provided herein is an antibody that binds TRDV2, comprising a heavy chain having an amino acid sequence having at least 95% identity to an amino acid sequence of SEQ ID NO:3059. In one aspect, provided herein is an antibody that binds TRDV2, comprising a light chain having an amino acid sequence having at least 95% identity to an amino acid sequence of SEQ ID NO:3060. In one aspect, provided herein is an antibody that binds TRDV2, comprising a heavy chain having an amino acid sequence having at least 95% identity to an amino acid sequence of SEQ ID NO:3059, and a light chain having an amino acid sequence having at least 95% identity to an amino acid sequence of SEQ ID NO:3060.

In one aspect, provided herein is an antibody that binds TRDV2, comprising: (i) a VH comprising a VH CDR1, a VH CDR2, and a VH CDR3 having an amino acid sequence of SEQ ID NOs:3061, 3062, and 3063, respectively, and (ii) a VL comprising a VL CDR1, VL CDR2, and VL CDR3 having an amino acid sequence of SEQ ID NOs:3064, 3065, and 3066, respectively. In one aspect, provided herein is an antibody that binds TRDV2, comprising: (i) a VH comprising a VH CDR1, a VH CDR2, and a VH CDR3 having an amino acid sequence of SEQ ID NOs:3067, 3068, and 3069, respectively, and (ii) a VL comprising a VL CDR1, VL CDR2, and VL CDR3 having an amino acid sequence of SEQ ID NOs:3070, 3071, and 3072, respectively. In one aspect, provided herein is an antibody that binds TRDV2, comprising: (i) a VH comprising a VH CDR1, a VH CDR2, and a VH CDR3 having an amino acid sequence of SEQ ID NOs:3073, 3074, and 3075, respectively, and (ii) a VL comprising a VL CDR1, VL CDR2, and VL CDR3 having an amino acid sequence of SEQ ID NOs:3076, 3077, and 3078, respectively. In one aspect, provided herein is an antibody that binds TRDV2, comprising: (i) a VH comprising a VH CDR1, a VH CDR2, and a VH CDR3 having an amino acid sequence of SEQ ID NOs:3079, 3080, and 3081, respectively, and (ii) a VL comprising a VL CDR1, VL CDR2, and VL CDR3 having an amino acid sequence of SEQ ID NOs:3082, 3083, and 3084, respectively. In one aspect, provided herein is an antibody that binds TRDV2, comprising: (i) a VH comprising a VH CDR1, a VH CDR2, and a VH CDR3 having an amino acid sequence of SEQ ID NOs:3085, 3086, and 3087, respectively, and (ii) a VL comprising a VL CDR1, VL CDR2, and VL CDR3 having an amino acid sequence of SEQ ID NOs:3088, 3089, and 3090, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3091; and (ii) a VL comprising a VL CDR1, a VL CDR2, and a VL CDR3 having an amino acid sequence of a VL CDR1, a VL CDR2, and a VL CDR3, respectively, of SEQ ID NO:3092. In one aspect, provided herein is an antibody that binds TRDV2, comprising a VH having an amino acid sequence of SEQ ID NO:3091. In one aspect, provided herein is an antibody that binds TRDV2, comprising a VL having an amino acid sequence of SEQ ID NO:3092. In one aspect, provided herein is an antibody that binds TRDV2, comprising a VH having an amino acid sequence of SEQ ID NO:3091, and a VL having an amino acid sequence of SEQ ID NO:3092. In one aspect, provided herein is an antibody that binds TRDV2, comprising a heavy chain having an amino acid sequence of SEQ ID NO:3093. In one aspect, provided herein is an antibody that binds TRDV2, comprising a light chain having an amino acid sequence of SEQ ID NO:3094. In one aspect, provided herein is an antibody that binds TRDV2, comprising a heavy chain having an amino acid sequence of SEQ ID NO:3093, and a light chain having an amino acid sequence of SEQ ID NO:3094. In one aspect, provided herein is an antibody that binds TRDV2, comprising a VH having an amino acid sequence having at least 95% identity to an amino acid sequence of SEQ ID NO:3091. In one aspect, provided herein is an antibody that binds TRDV2, comprising a VL having an amino acid sequence having at least 95% identity to an amino acid sequence of SEQ ID NO:3092. In one aspect, provided herein is an antibody that binds TRDV2, comprising a VH having an amino acid sequence having at least 95% identity to an amino acid sequence of SEQ ID NO:3091, and a VL having an amino acid sequence having at least 95% identity to an amino acid sequence of SEQ ID NO:3092. In one aspect, provided herein is an antibody that binds TRDV2, comprising a heavy chain having an amino acid sequence having at least 95% identity to an amino acid sequence of SEQ ID NO:3093. In one aspect, provided herein is an antibody that binds TRDV2, comprising a light chain having an amino acid sequence having at least 95% identity to an amino acid sequence of SEQ ID NO:3094. In one aspect, provided herein is an antibody that binds TRDV2, comprising a heavy chain having an amino acid sequence having at least 95% identity to an amino acid sequence of SEQ ID NO:3093, and a light chain having an amino acid sequence having at least 95% identity to an amino acid sequence of SEQ ID NO:3094.

In one aspect, provided herein is an antibody that binds TRDV2, comprising: (i) a VH comprising a VH CDR1, a VH CDR2, and a VH CDR3 having an amino acid sequence of SEQ ID NOs:3095, 3096, and 3097, respectively, and (ii) a VL comprising a VL CDR1, VL CDR2, and VL CDR3 having an amino acid sequence of SEQ ID NOs:3098, 3099, and 3100, respectively. In one aspect, provided herein is an antibody that binds TRDV2, comprising: (i) a VH comprising a VH CDR1, a VH CDR2, and a VH CDR3 having an amino acid sequence of SEQ ID NOs:3101, 3102, and 3103, respectively, and (ii) a VL comprising a VL CDR1, VL CDR2, and VL CDR3 having an amino acid sequence of SEQ ID NOs:3104, 3105, and 3106, respectively. In one aspect, provided herein is an antibody that binds TRDV2, comprising: (i) a VH comprising a VH CDR1, a VH CDR2, and a VH CDR3 having an amino acid sequence of SEQ ID NOs:3107, 3108, and 3109, respectively, and (ii) a VL comprising a VL CDR1, VL CDR2, and VL CDR3 having an amino acid sequence of SEQ ID NOs:3110, 3111, and 3112, respectively. In one aspect, provided herein is an antibody that binds TRDV2, comprising: (i) a VH comprising a VH CDR1, a VH CDR2, and a VH CDR3 having an amino acid sequence of SEQ ID NOs:3113, 3114, and 3115, respectively, and (ii) a VL comprising a VL CDR1, VL CDR2, and VL CDR3 having an amino acid sequence of SEQ ID NOs:3116, 3117, and 3118, respectively. In one aspect, provided herein is an antibody that binds TRDV2, comprising: (i) a VH comprising a VH CDR1, a VH CDR2, and a VH CDR3 having an amino acid sequence of SEQ ID NOs:3119, 3120, and 3121, respectively, and (ii) a VL comprising a VL CDR1, VL CDR2, and VL CDR3 having an amino acid sequence of SEQ ID NOs:3122, 3123, and 3124, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3125; and (ii) a VL comprising a VL CDR1, a VL CDR2, and a VL CDR3 having an amino acid sequence of a VL CDR1, a VL CDR2, and a VL CDR3, respectively, of SEQ ID NO:3126. In one aspect, provided herein is an antibody that binds TRDV2, comprising a VH having an amino acid sequence of SEQ ID NO:3125. In one aspect, provided herein is an antibody that binds TRDV2, comprising a VL having an amino acid sequence of SEQ ID NO:3126. In one aspect, provided herein is an antibody that binds TRDV2, comprising a VH having an amino acid sequence of SEQ ID NO:3125, and a VL having an amino acid sequence of SEQ ID NO:3126. In one aspect, provided herein is an antibody that binds TRDV2, comprising a heavy chain having an amino acid sequence of SEQ ID NO:3127. In one aspect, provided herein is an antibody that binds TRDV2, comprising a light chain having an amino acid sequence of SEQ ID NO:3128. In one aspect, provided herein is an antibody that binds TRDV2, comprising a heavy chain having an amino acid sequence of SEQ ID NO:3127, and a light chain having an amino acid sequence of SEQ ID NO:3128. In one aspect, provided herein is an antibody that binds TRDV2, comprising a VH having an amino acid sequence having at least 95% identity to an amino acid sequence of SEQ ID NO:3125. In one aspect, provided herein is an antibody that binds TRDV2, comprising a VL having an amino acid sequence having at least 95% identity to an amino acid sequence of SEQ ID NO:3126. In one aspect, provided herein is an antibody that binds TRDV2, comprising a VH having an amino acid sequence having at least 95% identity to an amino acid sequence of SEQ ID NO:3125, and a VL having an amino acid sequence having at least 95% identity to an amino acid sequence of SEQ ID NO:3126. In one aspect, provided herein is an antibody that binds TRDV2, comprising a heavy chain having an amino acid sequence having at least 95% identity to an amino acid sequence of SEQ ID NO:3127. In one aspect, provided herein is an antibody that binds TRDV2, comprising a light chain having an amino acid sequence having at least 95% identity to an amino acid sequence of SEQ ID NO:3128. In one aspect, provided herein is an antibody that binds TRDV2, comprising a heavy chain having an amino acid sequence having at least 95% identity to an amino acid sequence of SEQ ID NO:3127, and a light chain having an amino acid sequence having at least 95% identity to an amino acid sequence of SEQ ID NO:3128.

In one aspect, provided herein is an antibody that binds TRDV2, comprising: (i) a VH comprising a VH CDR1, a VH CDR2, and a VH CDR3 having an amino acid sequence of SEQ ID NOs:3129, 3130, and 3131, respectively, and (ii) a VL comprising a VL CDR1, VL CDR2, and VL CDR3 having an amino acid sequence of SEQ ID NOs:3132, 3133, and 3134, respectively. In one aspect, provided herein is an antibody that binds TRDV2, comprising: (i) a VH comprising a VH CDR1, a VH CDR2, and a VH CDR3 having an amino acid sequence of SEQ ID NOs:3135, 3136, and 3137, respectively, and (ii) a VL comprising a VL CDR1, VL CDR2, and VL CDR3 having an amino acid sequence of SEQ ID NOs:3138, 3139, and 3140, respectively. In one aspect, provided herein is an antibody that binds TRDV2, comprising: (i) a VH comprising a VH CDR1, a VH CDR2, and a VH CDR3 having an amino acid sequence of SEQ ID NOs:3141, 3142, and 3143, respectively, and (ii) a VL comprising a VL CDR1, VL CDR2, and VL CDR3 having an amino acid sequence of SEQ ID NOs:3144, 3145, and 3146, respectively. In one aspect, provided herein is an antibody that binds TRDV2, comprising: (i) a VH comprising a VH CDR1, a VH CDR2, and a VH CDR3 having an amino acid sequence of SEQ ID NOs:3147, 3148, and 3149, respectively, and (ii) a VL comprising a VL CDR1, VL CDR2, and VL CDR3 having an amino acid sequence of SEQ ID NOs:3150, 3151, and 3152, respectively. In one aspect, provided herein is an antibody that binds TRDV2, comprising: (i) a VH comprising a VH CDR1, a VH CDR2, and a VH CDR3 having an amino acid sequence of SEQ ID NOs:3153, 3154, and 3155, respectively, and (ii) a VL comprising a VL CDR1, VL CDR2, and VL CDR3 having an amino acid sequence of SEQ ID NOs:3156, 3157, and 3158, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3159; and (ii) a VL comprising a VL CDR1, a VL CDR2, and a VL CDR3 having an amino acid sequence of a VL CDR1, a VL CDR2, and a VL CDR3, respectively, of SEQ ID NO:3160. In one aspect, provided herein is an antibody that binds TRDV2, comprising a VH having an amino acid sequence of SEQ ID NO:3159. In one aspect, provided herein is an antibody that binds TRDV2, comprising a VL having an amino acid sequence of SEQ ID NO:3160. In one aspect, provided herein is an antibody that binds TRDV2, comprising a VH having an amino acid sequence of SEQ ID NO:3159, and a VL having an amino acid sequence of SEQ ID NO:3160. In one aspect, provided herein is an antibody that binds TRDV2, comprising a heavy chain having an amino acid sequence of SEQ ID NO:3161. In one aspect, provided herein is an antibody that binds TRDV2, comprising a light chain having an amino acid sequence of SEQ ID NO:3162. In one aspect, provided herein is an antibody that binds TRDV2, comprising a heavy chain having an amino acid sequence of SEQ ID NO:3161, and a light chain having an amino acid sequence of SEQ ID NO:3162. In one aspect, provided herein is an antibody that binds TRDV2, comprising a VH having an amino acid sequence having at least 95% identity to an amino acid sequence of SEQ ID NO:3159. In one aspect, provided herein is an antibody that binds TRDV2, comprising a VL having an amino acid sequence having at least 95% identity to an amino acid sequence of SEQ ID NO:3160. In one aspect, provided herein is an antibody that binds TRDV2, comprising a VH having an amino acid sequence having at least 95% identity to an amino acid sequence of SEQ ID NO:3159, and a VL having an amino acid sequence having at least 95% identity to an amino acid sequence of SEQ ID NO:3160. In one aspect, provided herein is an antibody that binds TRDV2, comprising a heavy chain having an amino acid sequence having at least 95% identity to an amino acid sequence of SEQ ID NO:3161. In one aspect, provided herein is an antibody that binds TRDV2, comprising a light chain having an amino acid sequence having at least 95% identity to an amino acid sequence of SEQ ID NO:3162. In one aspect, provided herein is an antibody that binds TRDV2, comprising a heavy chain having an amino acid sequence having at least 95% identity to an amino acid sequence of SEQ ID NO:3161, and a light chain having an amino acid sequence having at least 95% identity to an amino acid sequence of SEQ ID NO:3162.

In one aspect, provided herein is an antibody that binds TRDV2, comprising: (i) a VH comprising a VH CDR1, a VH CDR2, and a VH CDR3 having an amino acid sequence of SEQ ID NOs:3163, 3164, and 3165, respectively, and (ii) a VL comprising a VL CDR1, VL CDR2, and VL CDR3 having an amino acid sequence of SEQ ID NOs:3166, 3167, and 3168, respectively. In one aspect, provided herein is an antibody that binds TRDV2, comprising: (i) a VH comprising a VH CDR1, a VH CDR2, and a VH CDR3 having an amino acid sequence of SEQ ID NOs:3169, 3170, and 3171, respectively, and (ii) a VL comprising a VL CDR1, VL CDR2, and VL CDR3 having an amino acid sequence of SEQ ID NOs:3172, 3173, and 3174, respectively. In one aspect, provided herein is an antibody that binds TRDV2, comprising: (i) a VH comprising a VH CDR1, a VH CDR2, and a VH CDR3 having an amino acid sequence of SEQ ID NOs:3175, 3176, and 3177, respectively, and (ii) a VL comprising a VL CDR1, VL CDR2, and VL CDR3 having an amino acid sequence of SEQ ID NOs:3178, 3179, and 3180, respectively. In one aspect, provided herein is an antibody that binds TRDV2, comprising: (i) a VH comprising a VH CDR1, a VH CDR2, and a VH CDR3 having an amino acid sequence of SEQ ID NOs:3181, 3182, and 3183, respectively, and (ii) a VL comprising a VL CDR1, VL CDR2, and VL CDR3 having an amino acid sequence of SEQ ID NOs:3184, 3185, and 3186, respectively. In one aspect, provided herein is an antibody that binds TRDV2, comprising: (i) a VH comprising a VH CDR1, a VH CDR2, and a VH CDR3 having an amino acid sequence of SEQ ID NOs:3187, 3188, and 3189, respectively, and (ii) a VL comprising a VL CDR1, VL CDR2, and VL CDR3 having an amino acid sequence of SEQ ID NOs:3190, 3191, and 3192, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3193; and (ii) a VL comprising a VL CDR1, a VL CDR2, and a VL CDR3 having an amino acid sequence of a VL CDR1, a VL CDR2, and a VL CDR3, respectively, of SEQ ID NO:3194. In one aspect, provided herein is an antibody that binds TRDV2, comprising a VH having an amino acid sequence of SEQ ID NO:3193. In one aspect, provided herein is an antibody that binds TRDV2, comprising a VL having an amino acid sequence of SEQ ID NO:3194. In one aspect, provided herein is an antibody that binds TRDV2, comprising a VH having an amino acid sequence of SEQ ID NO:3193, and a VL having an amino acid sequence of SEQ ID NO:3194. In one aspect, provided herein is an antibody that binds TRDV2, comprising a heavy chain having an amino acid sequence of SEQ ID NO:3195. In one aspect, provided herein is an antibody that binds TRDV2, comprising a light chain having an amino acid sequence of SEQ ID NO:3196. In one aspect, provided herein is an antibody that binds TRDV2, comprising a heavy chain having an amino acid sequence of SEQ ID NO:3195, and a light chain having an amino acid sequence of SEQ ID NO:3196. In one aspect, provided herein is an antibody that binds TRDV2, comprising a VH having an amino acid sequence having at least 95% identity to an amino acid sequence of SEQ ID NO:3193. In one aspect, provided herein is an antibody that binds TRDV2, comprising a VL having an amino acid sequence having at least 95% identity to an amino acid sequence of SEQ ID NO:3194. In one aspect, provided herein is an antibody that binds TRDV2, comprising a VH having an amino acid sequence having at least 95% identity to an amino acid sequence of SEQ ID NO:3193, and a VL having an amino acid sequence having at least 95% identity to an amino acid sequence of SEQ ID NO:3194. In one aspect, provided herein is an antibody that binds TRDV2, comprising a heavy chain having an amino acid sequence having at least 95% identity to an amino acid sequence of SEQ ID NO:3195. In one aspect, provided herein is an antibody that binds TRDV2, comprising a light chain having an amino acid sequence having at least 95% identity to an amino acid sequence of SEQ ID NO:3196. In one aspect, provided herein is an antibody that binds TRDV2, comprising a heavy chain having an amino acid sequence having at least 95% identity to an amino acid sequence of SEQ ID NO:3195, and a light chain having an amino acid sequence having at least 95% identity to an amino acid sequence of SEQ ID NO:3196.

In one aspect, provided herein is an antibody that binds TRDV2, comprising: (i) a VH comprising a VH CDR1, a VH CDR2, and a VH CDR3 having an amino acid sequence of SEQ ID NOs:3197, 3198, and 3199, respectively, and (ii) a VL comprising a VL CDR1, VL CDR2, and VL CDR3 having an amino acid sequence of SEQ ID NOs:3200, 3201, and 3202, respectively. In one aspect, provided herein is an antibody that binds TRDV2, comprising: (i) a VH comprising a VH CDR1, a VH CDR2, and a VH CDR3 having an amino acid sequence of SEQ ID NOs:3203, 3204, and 3205, respectively, and (ii) a VL comprising a VL CDR1, VL CDR2, and VL CDR3 having an amino acid sequence of SEQ ID NOs:3206, 3207, and 3208, respectively. In one aspect, provided herein is an antibody that binds TRDV2, comprising: (i) a VH comprising a VH CDR1, a VH CDR2, and a VH CDR3 having an amino acid sequence of SEQ ID NOs:3209, 3210, and 3211, respectively, and (ii) a VL comprising a VL CDR1, VL CDR2, and VL CDR3 having an amino acid sequence of SEQ ID NOs:3212, 3213, and 3214, respectively. In one aspect, provided herein is an antibody that binds TRDV2, comprising: (i) a VH comprising a VH CDR1, a VH CDR2, and a VH CDR3 having an amino acid sequence of SEQ ID NOs:3215, 3216, and 3217, respectively, and (ii) a VL comprising a VL CDR1, VL CDR2, and VL CDR3 having an amino acid sequence of SEQ ID NOs:3218, 3219, and 3220, respectively. In one aspect, provided herein is an antibody that binds TRDV2, comprising: (i) a VH comprising a VH CDR1, a VH CDR2, and a VH CDR3 having an amino acid sequence of SEQ ID NOs:3221, 3222, and 3223, respectively, and (ii) a VL comprising a VL CDR1, VL CDR2, and VL CDR3 having an amino acid sequence of SEQ ID NOs:3224, 3225, and 3226, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3227; and (ii) a VL comprising a VL CDR1, a VL CDR2, and a VL CDR3 having an amino acid sequence of a VL CDR1, a VL CDR2, and a VL CDR3, respectively, of SEQ ID NO:3228. In one aspect, provided herein is an antibody that binds TRDV2, comprising a VH having an amino acid sequence of SEQ ID NO:3227. In one aspect, provided herein is an antibody that binds TRDV2, comprising a VL having an amino acid sequence of SEQ ID NO:3228. In one aspect, provided herein is an antibody that binds TRDV2, comprising a VH having an amino acid sequence of SEQ ID NO:3227, and a VL having an amino acid sequence of SEQ ID NO:3228. In one aspect, provided herein is an antibody that binds TRDV2, comprising a heavy chain having an amino acid sequence of SEQ ID NO:3229. In one aspect, provided herein is an antibody that binds TRDV2, comprising a light chain having an amino acid sequence of SEQ ID NO:3230. In one aspect, provided herein is an antibody that binds TRDV2, comprising a heavy chain having an amino acid sequence of SEQ ID NO:3229, and a light chain having an amino acid sequence of SEQ ID NO:3230. In one aspect, provided herein is an antibody that binds TRDV2, comprising a VH having an amino acid sequence having at least 95% identity to an amino acid sequence of SEQ ID NO:3227. In one aspect, provided herein is an antibody that binds TRDV2, comprising a VL having an amino acid sequence having at least 95% identity to an amino acid sequence of SEQ ID NO:3228. In one aspect, provided herein is an antibody that binds TRDV2, comprising a VH having an amino acid sequence having at least 95% identity to an amino acid sequence of SEQ ID NO:3227, and a VL having an amino acid sequence having at least 95% identity to an amino acid sequence of SEQ ID NO:3228. In one aspect, provided herein is an antibody that binds TRDV2, comprising a heavy chain having an amino acid sequence having at least 95% identity to an amino acid sequence of SEQ ID NO:3229. In one aspect, provided herein is an antibody that binds TRDV2, comprising a light chain having an amino acid sequence having at least 95% identity to an amino acid sequence of SEQ ID NO:3230. In one aspect, provided herein is an antibody that binds TRDV2, comprising a heavy chain having an amino acid sequence having at least 95% identity to an amino acid sequence of SEQ ID NO:3229, and a light chain having an amino acid sequence having at least 95% identity to an amino acid sequence of SEQ ID NO:3230.

In one aspect, provided herein is an antibody that binds TRDV2, comprising: (i) a VH comprising a VH CDR1, a VH CDR2, and a VH CDR3 having an amino acid sequence of SEQ ID NOs:3231, 3232, and 3233, respectively, and (ii) a VL comprising a VL CDR1, VL CDR2, and VL CDR3 having an amino acid sequence of SEQ ID NOs:3234, 3235, and 3236, respectively. In one aspect, provided herein is an antibody that binds TRDV2, comprising: (i) a VH comprising a VH CDR1, a VH CDR2, and a VH CDR3 having an amino acid sequence of SEQ ID NOs:3237, 3238, and 3239, respectively, and (ii) a VL comprising a VL CDR1, VL CDR2, and VL CDR3 having an amino acid sequence of SEQ ID NOs:3240, 3241, and 3242, respectively. In one aspect, provided herein is an antibody that binds TRDV2, comprising: (i) a VH comprising a VH CDR1, a VH CDR2, and a VH CDR3 having an amino acid sequence of SEQ ID NOs:3243, 3244, and 3245, respectively, and (ii) a VL comprising a VL CDR1, VL CDR2, and VL CDR3 having an amino acid sequence of SEQ ID NOs:3246, 3247, and 3248, respectively. In one aspect, provided herein is an antibody that binds TRDV2, comprising: (i) a VH comprising a VH CDR1, a VH CDR2, and a VH CDR3 having an amino acid sequence of SEQ ID NOs:3249, 3250, and 3251, respectively, and (ii) a VL comprising a VL CDR1, VL CDR2, and VL CDR3 having an amino acid sequence of SEQ ID NOs:3252, 3253, and 3254, respectively. In one aspect, provided herein is an antibody that binds TRDV2, comprising: (i) a VH comprising a VH CDR1, a VH CDR2, and a VH CDR3 having an amino acid sequence of SEQ ID NOs:3255, 3256, and 3257, respectively, and (ii) a VL comprising a VL CDR1, VL CDR2, and VL CDR3 having an amino acid sequence of SEQ ID NOs:3258, 3259, and 3260, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3261; and (ii) a VL comprising a VL CDR1, a VL CDR2, and a VL CDR3 having an amino acid sequence of a VL CDR1, a VL CDR2, and a VL CDR3, respectively, of SEQ ID NO:3262. In one aspect, provided herein is an antibody that binds TRDV2, comprising a VH having an amino acid sequence of SEQ ID NO:3261. In one aspect, provided herein is an antibody that binds TRDV2, comprising a VL having an amino acid sequence of SEQ ID NO:3262. In one aspect, provided herein is an antibody that binds TRDV2, comprising a VH having an amino acid sequence of SEQ ID NO:3261, and a VL having an amino acid sequence of SEQ ID NO:3262. In one aspect, provided herein is an antibody that binds TRDV2, comprising a heavy chain having an amino acid sequence of SEQ ID NO:3263. In one aspect, provided herein is an antibody that binds TRDV2, comprising a light chain having an amino acid sequence of SEQ ID NO:3264. In one aspect, provided herein is an antibody that binds TRDV2, comprising a heavy chain having an amino acid sequence of SEQ ID NO:3263, and a light chain having an amino acid sequence of SEQ ID NO:3264. In one aspect, provided herein is an antibody that binds TRDV2, comprising a VH having an amino acid sequence having at least 95% identity to an amino acid sequence of SEQ ID NO:3261. In one aspect, provided herein is an antibody that binds TRDV2, comprising a VL having an amino acid sequence having at least 95% identity to an amino acid sequence of SEQ ID NO:3262. In one aspect, provided herein is an antibody that binds TRDV2, comprising a VH having an amino acid sequence having at least 95% identity to an amino acid sequence of SEQ ID NO:3261, and a VL having an amino acid sequence having at least 95% identity to an amino acid sequence of SEQ ID NO:3262. In one aspect, provided herein is an antibody that binds TRDV2, comprising a heavy chain having an amino acid sequence having at least 95% identity to an amino acid sequence of SEQ ID NO:3263. In one aspect, provided herein is an antibody that binds TRDV2, comprising a light chain having an amino acid sequence having at least 95% identity to an amino acid sequence of SEQ ID NO:3264. In one aspect, provided herein is an antibody that binds TRDV2, comprising a heavy chain having an amino acid sequence having at least 95% identity to an amino acid sequence of SEQ ID NO:3263, and a light chain having an amino acid sequence having at least 95% identity to an amino acid sequence of SEQ ID NO:3264.

In one aspect, provided herein is an antibody that binds TRDV2, comprising: (i) a VH comprising a VH CDR1, a VH CDR2, and a VH CDR3 having an amino acid sequence of SEQ ID NOs:3265, 3266, and 3267, respectively, and (ii) a VL comprising a VL CDR1, VL CDR2, and VL CDR3 having an amino acid sequence of SEQ ID NOs:3268, 3269, and 3270, respectively. In one aspect, provided herein is an antibody that binds TRDV2, comprising: (i) a VH comprising a VH CDR1, a VH CDR2, and a VH CDR3 having an amino acid sequence of SEQ ID NOs:3271, 3272, and 3273, respectively, and (ii) a VL comprising a VL CDR1, VL CDR2, and VL CDR3 having an amino acid sequence of SEQ ID NOs:3274, 3275, and 3276, respectively. In one aspect, provided herein is an antibody that binds TRDV2, comprising: (i) a VH comprising a VH CDR1, a VH CDR2, and a VH CDR3 having an amino acid sequence of SEQ ID NOs:3277, 3278, and 3279, respectively, and (ii) a VL comprising a VL CDR1, VL CDR2, and VL CDR3 having an amino acid sequence of SEQ ID NOs:3280, 3281, and 3282, respectively. In one aspect, provided herein is an antibody that binds TRDV2, comprising: (i) a VH comprising a VH CDR1, a VH CDR2, and a VH CDR3 having an amino acid sequence of SEQ ID NOs:3283, 3284, and 3285, respectively, and (ii) a VL comprising a VL CDR1, VL CDR2, and VL CDR3 having an amino acid sequence of SEQ ID NOs:3286, 3287, and 3288, respectively. In one aspect, provided herein is an antibody that binds TRDV2, comprising: (i) a VH comprising a VH CDR1, a VH CDR2, and a VH CDR3 having an amino acid sequence of SEQ ID NOs:3289, 3290, and 3291, respectively, and (ii) a VL comprising a VL CDR1, VL CDR2, and VL CDR3 having an amino acid sequence of SEQ ID NOs:3292, 3293, and 3294, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3295; and (ii) a VL comprising a VL CDR1, a VL CDR2, and a VL CDR3 having an amino acid sequence of a VL CDR1, a VL CDR2, and a VL CDR3, respectively, of SEQ ID NO:3296. In one aspect, provided herein is an antibody that binds TRDV2, comprising a VH having an amino acid sequence of SEQ ID NO:3295. In one aspect, provided herein is an antibody that binds TRDV2, comprising a VL having an amino acid sequence of SEQ ID NO:3296. In one aspect, provided herein is an antibody that binds TRDV2, comprising a VH having an amino acid sequence of SEQ ID NO:3295, and a VL having an amino acid sequence of SEQ ID NO:3296. In one aspect, provided herein is an antibody that binds TRDV2, comprising a heavy chain having an amino acid sequence of SEQ ID NO:3297. In one aspect, provided herein is an antibody that binds TRDV2, comprising a light chain having an amino acid sequence of SEQ ID NO:3298. In one aspect, provided herein is an antibody that binds TRDV2, comprising a heavy chain having an amino acid sequence of SEQ ID NO:3297, and a light chain having an amino acid sequence of SEQ ID NO:3298. In one aspect, provided herein is an antibody that binds TRDV2, comprising a VH having an amino acid sequence having at least 95% identity to an amino acid sequence of SEQ ID NO:3295. In one aspect, provided herein is an antibody that binds TRDV2, comprising a VL having an amino acid sequence having at least 95% identity to an amino acid sequence of SEQ ID NO:3296. In one aspect, provided herein is an antibody that binds TRDV2, comprising a VH having an amino acid sequence having at least 95% identity to an amino acid sequence of SEQ ID NO:3295, and a VL having an amino acid sequence having at least 95% identity to an amino acid sequence of SEQ ID NO:3296. In one aspect, provided herein is an antibody that binds TRDV2, comprising a heavy chain having an amino acid sequence having at least 95% identity to an amino acid sequence of SEQ ID NO:3297. In one aspect, provided herein is an antibody that binds TRDV2, comprising a light chain having an amino acid sequence having at least 95% identity to an amino acid sequence of SEQ ID NO:3298. In one aspect, provided herein is an antibody that binds TRDV2, comprising a heavy chain having an amino acid sequence having at least 95% identity to an amino acid sequence of SEQ ID NO:3297, and a light chain having an amino acid sequence having at least 95% identity to an amino acid sequence of SEQ ID NO:3298.

In one aspect, provided herein is an antibody that binds TRDV2, comprising: (i) a VH comprising a VH CDR1, a VH CDR2, and a VH CDR3 having an amino acid sequence of SEQ ID NOs:3299, 3300, and 3301, respectively, and (ii) a VL comprising a VL CDR1, VL CDR2, and VL CDR3 having an amino acid sequence of SEQ ID NOs:3302, 3303, and 3304, respectively. In one aspect, provided herein is an antibody that binds TRDV2, comprising: (i) a VH comprising a VH CDR1, a VH CDR2, and a VH CDR3 having an amino acid sequence of SEQ ID NOs:3305, 3306, and 3307, respectively, and (ii) a VL comprising a VL CDR1, VL CDR2, and VL CDR3 having an amino acid sequence of SEQ ID NOs:3308, 3309, and 3310, respectively. In one aspect, provided herein is an antibody that binds TRDV2, comprising: (i) a VH comprising a VH CDR1, a VH CDR2, and a VH CDR3 having an amino acid sequence of SEQ ID NOs:3311, 3312, and 3313, respectively, and (ii) a VL comprising a VL CDR1, VL CDR2, and VL CDR3 having an amino acid sequence of SEQ ID NOs:3314, 3315, and 3316, respectively. In one aspect, provided herein is an antibody that binds TRDV2, comprising: (i) a VH comprising a VH CDR1, a VH CDR2, and a VH CDR3 having an amino acid sequence of SEQ ID NOs:3317, 3318, and 3319, respectively, and (ii) a VL comprising a VL CDR1, VL CDR2, and VL CDR3 having an amino acid sequence of SEQ ID NOs:3320, 3321, and 3322, respectively. In one aspect, provided herein is an antibody that binds TRDV2, comprising: (i) a VH comprising a VH CDR1, a VH CDR2, and a VH CDR3 having an amino acid sequence of SEQ ID NOs:3323, 3324, and 3325, respectively, and (ii) a VL comprising a VL CDR1, VL CDR2, and VL CDR3 having an amino acid sequence of SEQ ID NOs:3326, 3327, and 3328, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3329; and (ii) a VL comprising a VL CDR1, a VL CDR2, and a VL CDR3 having an amino acid sequence of a VL CDR1, a VL CDR2, and a VL CDR3, respectively, of SEQ ID NO:3330. In one aspect, provided herein is an antibody that binds TRDV2, comprising a VH having an amino acid sequence of SEQ ID NO:3329. In one aspect, provided herein is an antibody that binds TRDV2, comprising a VL having an amino acid sequence of SEQ ID NO:3330. In one aspect, provided herein is an antibody that binds TRDV2, comprising a VH having an amino acid sequence of SEQ ID NO:3329, and a VL having an amino acid sequence of SEQ ID NO:3330. In one aspect, provided herein is an antibody that binds TRDV2, comprising a heavy chain having an amino acid sequence of SEQ ID NO:3331. In one aspect, provided herein is an antibody that binds TRDV2, comprising a light chain having an amino acid sequence of SEQ ID NO:3332. In one aspect, provided herein is an antibody that binds TRDV2, comprising a heavy chain having an amino acid sequence of SEQ ID NO:3331, and a light chain having an amino acid sequence of SEQ ID NO:3332. In one aspect, provided herein is an antibody that binds TRDV2, comprising a VH having an amino acid sequence having at least 95% identity to an amino acid sequence of SEQ ID NO:3329. In one aspect, provided herein is an antibody that binds TRDV2, comprising a VL having an amino acid sequence having at least 95% identity to an amino acid sequence of SEQ ID NO:3330. In one aspect, provided herein is an antibody that binds TRDV2, comprising a VH having an amino acid sequence having at least 95% identity to an amino acid sequence of SEQ ID NO:3329, and a VL having an amino acid sequence having at least 95% identity to an amino acid sequence of SEQ ID NO:3330. In one aspect, provided herein is an antibody that binds TRDV2, comprising a heavy chain having an amino acid sequence having at least 95% identity to an amino acid sequence of SEQ ID NO:3331. In one aspect, provided herein is an antibody that binds TRDV2, comprising a light chain having an amino acid sequence having at least 95% identity to an amino acid sequence of SEQ ID NO:3332. In one aspect, provided herein is an antibody that binds TRDV2, comprising a heavy chain having an amino acid sequence having at least 95% identity to an amino acid sequence of SEQ ID NO:3331, and a light chain having an amino acid sequence having at least 95% identity to an amino acid sequence of SEQ ID NO:3332.

In one aspect, provided herein is an antibody that binds TRDV2, comprising: (i) a VH comprising a VH CDR1, a VH CDR2, and a VH CDR3 having an amino acid sequence of SEQ ID NOs:3333, 3334, and 3335, respectively, and (ii) a VL comprising a VL CDR1, VL CDR2, and VL CDR3 having an amino acid sequence of SEQ ID NOs:3336, 3337, and 3338, respectively. In one aspect, provided herein is an antibody that binds TRDV2, comprising: (i) a VH comprising a VH CDR1, a VH CDR2, and a VH CDR3 having an amino acid sequence of SEQ ID NOs:3339, 3340, and 3341, respectively, and (ii) a VL comprising a VL CDR1, VL CDR2, and VL CDR3 having an amino acid sequence of SEQ ID NOs:3342, 3343, and 3344, respectively. In one aspect, provided herein is an antibody that binds TRDV2, comprising: (i) a VH comprising a VH CDR1, a VH CDR2, and a VH CDR3 having an amino acid sequence of SEQ ID NOs:3345, 3346, and 3347, respectively, and (ii) a VL comprising a VL CDR1, VL CDR2, and VL CDR3 having an amino acid sequence of SEQ ID NOs:3348, 3349, and 3350, respectively. In one aspect, provided herein is an antibody that binds TRDV2, comprising: (i) a VH comprising a VH CDR1, a VH CDR2, and a VH CDR3 having an amino acid sequence of SEQ ID NOs:3351, 3352, and 3353, respectively, and (ii) a VL comprising a VL CDR1, VL CDR2, and VL CDR3 having an amino acid sequence of SEQ ID NOs:3354, 3355, and 3356, respectively. In one aspect, provided herein is an antibody that binds TRDV2, comprising: (i) a VH comprising a VH CDR1, a VH CDR2, and a VH CDR3 having an amino acid sequence of SEQ ID NOs:3357, 3358, and 3359, respectively, and (ii) a VL comprising a VL CDR1, VL CDR2, and VL CDR3 having an amino acid sequence of SEQ ID NOs:3360, 3361, and 3362, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3363; and (ii) a VL comprising a VL CDR1, a VL CDR2, and a VL CDR3 having an amino acid sequence of a VL CDR1, a VL CDR2, and a VL CDR3, respectively, of SEQ ID NO:3364. In one aspect, provided herein is an antibody that binds TRDV2, comprising a VH having an amino acid sequence of SEQ ID NO:3363. In one aspect, provided herein is an antibody that binds TRDV2, comprising a VL having an amino acid sequence of SEQ ID NO:3364. In one aspect, provided herein is an antibody that binds TRDV2, comprising a VH having an amino acid sequence of SEQ ID NO:3363, and a VL having an amino acid sequence of SEQ ID NO:3364. In one aspect, provided herein is an antibody that binds TRDV2, comprising a heavy chain having an amino acid sequence of SEQ ID NO:3365. In one aspect, provided herein is an antibody that binds TRDV2, comprising a light chain having an amino acid sequence of SEQ ID NO:3366. In one aspect, provided herein is an antibody that binds TRDV2, comprising a heavy chain having an amino acid sequence of SEQ ID NO:3365, and a light chain having an amino acid sequence of SEQ ID NO:3366. In one aspect, provided herein is an antibody that binds TRDV2, comprising a VH having an amino acid sequence having at least 95% identity to an amino acid sequence of SEQ ID NO:3363. In one aspect, provided herein is an antibody that binds TRDV2, comprising a VL having an amino acid sequence having at least 95% identity to an amino acid sequence of SEQ ID NO:3364. In one aspect, provided herein is an antibody that binds TRDV2, comprising a VH having an amino acid sequence having at least 95% identity to an amino acid sequence of SEQ ID NO:3363, and a VL having an amino acid sequence having at least 95% identity to an amino acid sequence of SEQ ID NO:3364. In one aspect, provided herein is an antibody that binds TRDV2, comprising a heavy chain having an amino acid sequence having at least 95% identity to an amino acid sequence of SEQ ID NO:3365. In one aspect, provided herein is an antibody that binds TRDV2, comprising a light chain having an amino acid sequence having at least 95% identity to an amino acid sequence of SEQ ID NO:3366. In one aspect, provided herein is an antibody that binds TRDV2, comprising a heavy chain having an amino acid sequence having at least 95% identity to an amino acid sequence of SEQ ID NO:3365, and a light chain having an amino acid sequence having at least 95% identity to an amino acid sequence of SEQ ID NO:3366.

In one aspect, provided herein is an antibody that binds TRDV2, comprising: (i) a VH comprising a VH CDR1, a VH CDR2, and a VH CDR3 having an amino acid sequence of SEQ ID NOs:3367, 3368, and 3369, respectively, and (ii) a VL comprising a VL CDR1, VL CDR2, and VL CDR3 having an amino acid sequence of SEQ ID NOs:3370, 3371, and 3372, respectively. In one aspect, provided herein is an antibody that binds TRDV2, comprising: (i) a VH comprising a VH CDR1, a VH CDR2, and a VH CDR3 having an amino acid sequence of SEQ ID NOs:3373, 3374, and 3375, respectively, and (ii) a VL comprising a VL CDR1, VL CDR2, and VL CDR3 having an amino acid sequence of SEQ ID NOs:3376, 3377, and 3378, respectively. In one aspect, provided herein is an antibody that binds TRDV2, comprising: (i) a VH comprising a VH CDR1, a VH CDR2, and a VH CDR3 having an amino acid sequence of SEQ ID NOs:3379, 3380, and 3381, respectively, and (ii) a VL comprising a VL CDR1, VL CDR2, and VL CDR3 having an amino acid sequence of SEQ ID NOs:3382, 3383, and 3384, respectively. In one aspect, provided herein is an antibody that binds TRDV2, comprising: (i) a VH comprising a VH CDR1, a VH CDR2, and a VH CDR3 having an amino acid sequence of SEQ ID NOs:3385, 3386, and 3387, respectively, and (ii) a VL comprising a VL CDR1, VL CDR2, and VL CDR3 having an amino acid sequence of SEQ ID NOs:3388, 3389, and 3390, respectively. In one aspect, provided herein is an antibody that binds TRDV2, comprising: (i) a VH comprising a VH CDR1, a VH CDR2, and a VH CDR3 having an amino acid sequence of SEQ ID NOs:3391, 3392, and 3393, respectively, and (ii) a VL comprising a VL CDR1, VL CDR2, and VL CDR3 having an amino acid sequence of SEQ ID NOs:3394, 3395, and 3396, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3397; and (ii) a VL comprising a VL CDR1, a VL CDR2, and a VL CDR3 having an amino acid sequence of a VL CDR1, a VL CDR2, and a VL CDR3, respectively, of SEQ ID NO:3398. In one aspect, provided herein is an antibody that binds TRDV2, comprising a VH having an amino acid sequence of SEQ ID NO:3397. In one aspect, provided herein is an antibody that binds TRDV2, comprising a VL having an amino acid sequence of SEQ ID NO:3398. In one aspect, provided herein is an antibody that binds TRDV2, comprising a VH having an amino acid sequence of SEQ ID NO:3397, and a VL having an amino acid sequence of SEQ ID NO:3398. In one aspect, provided herein is an antibody that binds TRDV2, comprising a heavy chain having an amino acid sequence of SEQ ID NO:3399. In one aspect, provided herein is an antibody that binds TRDV2, comprising a light chain having an amino acid sequence of SEQ ID NO:3400. In one aspect, provided herein is an antibody that binds TRDV2, comprising a heavy chain having an amino acid sequence of SEQ ID NO:3399, and a light chain having an amino acid sequence of SEQ ID NO:3400. In one aspect, provided herein is an antibody that binds TRDV2, comprising a VH having an amino acid sequence having at least 95% identity to an amino acid sequence of SEQ ID NO:3397. In one aspect, provided herein is an antibody that binds TRDV2, comprising a VL having an amino acid sequence having at least 95% identity to an amino acid sequence of SEQ ID NO:3398. In one aspect, provided herein is an antibody that binds TRDV2, comprising a VH having an amino acid sequence having at least 95% identity to an amino acid sequence of SEQ ID NO:3397, and a VL having an amino acid sequence having at least 95% identity to an amino acid sequence of SEQ ID NO:3398. In one aspect, provided herein is an antibody that binds TRDV2, comprising a heavy chain having an amino acid sequence having at least 95% identity to an amino acid sequence of SEQ ID NO:3399. In one aspect, provided herein is an antibody that binds TRDV2, comprising a light chain having an amino acid sequence having at least 95% identity to an amino acid sequence of SEQ ID NO:3400. In one aspect, provided herein is an antibody that binds TRDV2, comprising a heavy chain having an amino acid sequence having at least 95% identity to an amino acid sequence of SEQ ID NO:3399, and a light chain having an amino acid sequence having at least 95% identity to an amino acid sequence of SEQ ID NO:3400.

In one aspect, provided herein is an antibody that binds TRDV2, comprising: (i) a VH comprising a VH CDR1, a VH CDR2, and a VH CDR3 having an amino acid sequence of SEQ ID NOs:3401, 3402, and 3403, respectively, and (ii) a VL comprising a VL CDR1, VL CDR2, and VL CDR3 having an amino acid sequence of SEQ ID NOs:3404, 3405, and 3406, respectively. In one aspect, provided herein is an antibody that binds TRDV2, comprising: (i) a VH comprising a VH CDR1, a VH CDR2, and a VH CDR3 having an amino acid sequence of SEQ ID NOs:3407, 3408, and 3409, respectively, and (ii) a VL comprising a VL CDR1, VL CDR2, and VL CDR3 having an amino acid sequence of SEQ ID NOs:3410, 3411, and 3412, respectively. In one aspect, provided herein is an antibody that binds TRDV2, comprising: (i) a VH comprising a VH CDR1, a VH CDR2, and a VH CDR3 having an amino acid sequence of SEQ ID NOs:3413, 3414, and 3415, respectively, and (ii) a VL comprising a VL CDR1, VL CDR2, and VL CDR3 having an amino acid sequence of SEQ ID NOs:3416, 3417, and 3418, respectively. In one aspect, provided herein is an antibody that binds TRDV2, comprising: (i) a VH comprising a VH CDR1, a VH CDR2, and a VH CDR3 having an amino acid sequence of SEQ ID NOs:3419, 3420, and 3421, respectively, and (ii) a VL comprising a VL CDR1, VL CDR2, and VL CDR3 having an amino acid sequence of SEQ ID NOs:3422, 3423, and 3424, respectively. In one aspect, provided herein is an antibody that binds TRDV2, comprising: (i) a VH comprising a VH CDR1, a VH CDR2, and a VH CDR3 having an amino acid sequence of SEQ ID NOs:3425, 3426, and 3427, respectively, and (ii) a VL comprising a VL CDR1, VL CDR2, and VL CDR3 having an amino acid sequence of SEQ ID NOs:3428, 3429, and 3430, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3431; and (ii) a VL comprising a VL CDR1, a VL CDR2, and a VL CDR3 having an amino acid sequence of a VL CDR1, a VL CDR2, and a VL CDR3, respectively, of SEQ ID NO:3432. In one aspect, provided herein is an antibody that binds TRDV2, comprising a VH having an amino acid sequence of SEQ ID NO:3431. In one aspect, provided herein is an antibody that binds TRDV2, comprising a VL having an amino acid sequence of SEQ ID NO:3432. In one aspect, provided herein is an antibody that binds TRDV2, comprising a VH having an amino acid sequence of SEQ ID NO:3431, and a VL having an amino acid sequence of SEQ ID NO:3432. In one aspect, provided herein is an antibody that binds TRDV2, comprising a heavy chain having an amino acid sequence of SEQ ID NO:3433. In one aspect, provided herein is an antibody that binds TRDV2, comprising a light chain having an amino acid sequence of SEQ ID NO:3434. In one aspect, provided herein is an antibody that binds TRDV2, comprising a heavy chain having an amino acid sequence of SEQ ID NO:3433, and a light chain having an amino acid sequence of SEQ ID NO:3434. In one aspect, provided herein is an antibody that binds TRDV2, comprising a VH having an amino acid sequence having at least 95% identity to an amino acid sequence of SEQ ID NO:3431. In one aspect, provided herein is an antibody that binds TRDV2, comprising a VL having an amino acid sequence having at least 95% identity to an amino acid sequence of SEQ ID NO:3432. In one aspect, provided herein is an antibody that binds TRDV2, comprising a VH having an amino acid sequence having at least 95% identity to an amino acid sequence of SEQ ID NO:3431, and a VL having an amino acid sequence having at least 95% identity to an amino acid sequence of SEQ ID NO:3432. In one aspect, provided herein is an antibody that binds TRDV2, comprising a heavy chain having an amino acid sequence having at least 95% identity to an amino acid sequence of SEQ ID NO:3433. In one aspect, provided herein is an antibody that binds TRDV2, comprising a light chain having an amino acid sequence having at least 95% identity to an amino acid sequence of SEQ ID NO:3434. In one aspect, provided herein is an antibody that binds TRDV2, comprising a heavy chain having an amino acid sequence having at least 95% identity to an amino acid sequence of SEQ ID NO:3433, and a light chain having an amino acid sequence having at least 95% identity to an amino acid sequence of SEQ ID NO:3434.

In one aspect, provided herein is an antibody that binds TRDV2, comprising: (i) a VH comprising a VH CDR1, a VH CDR2, and a VH CDR3 having an amino acid sequence of SEQ ID NOs:3435, 3436, and 3437, respectively, and (ii) a VL comprising a VL CDR1, VL CDR2, and VL CDR3 having an amino acid sequence of SEQ ID NOs:3438, 3439, and 3440, respectively. In one aspect, provided herein is an antibody that binds TRDV2, comprising: (i) a VH comprising a VH CDR1, a VH CDR2, and a VH CDR3 having an amino acid sequence of SEQ ID NOs:3441, 3442, and 3443, respectively, and (ii) a VL comprising a VL CDR1, VL CDR2, and VL CDR3 having an amino acid sequence of SEQ ID NOs:3444, 3445, and 3446, respectively. In one aspect, provided herein is an antibody that binds TRDV2, comprising: (i) a VH comprising a VH CDR1, a VH CDR2, and a VH CDR3 having an amino acid sequence of SEQ ID NOs:3447, 3448, and 3449, respectively, and (ii) a VL comprising a VL CDR1, VL CDR2, and VL CDR3 having an amino acid sequence of SEQ ID NOs:3450, 3451, and 3452, respectively. In one aspect, provided herein is an antibody that binds TRDV2, comprising: (i) a VH comprising a VH CDR1, a VH CDR2, and a VH CDR3 having an amino acid sequence of SEQ ID NOs:3453, 3454, and 3455, respectively, and (ii) a VL comprising a VL CDR1, VL CDR2, and VL CDR3 having an amino acid sequence of SEQ ID NOs:3456, 3457, and 3458, respectively. In one aspect, provided herein is an antibody that binds TRDV2, comprising: (i) a VH comprising a VH CDR1, a VH CDR2, and a VH CDR3 having an amino acid sequence of SEQ ID NOs:3459, 3460, and 3461, respectively, and (ii) a VL comprising a VL CDR1, VL CDR2, and VL CDR3 having an amino acid sequence of SEQ ID NOs:3462, 3463, and 3464, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3465; and (ii) a VL comprising a VL CDR1, a VL CDR2, and a VL CDR3 having an amino acid sequence of a VL CDR1, a VL CDR2, and a VL CDR3, respectively, of SEQ ID NO:3466. In one aspect, provided herein is an antibody that binds TRDV2, comprising a VH having an amino acid sequence of SEQ ID NO:3465. In one aspect, provided herein is an antibody that binds TRDV2, comprising a VL having an amino acid sequence of SEQ ID NO:3466. In one aspect, provided herein is an antibody that binds TRDV2, comprising a VH having an amino acid sequence of SEQ ID NO:3465, and a VL having an amino acid sequence of SEQ ID NO:3466. In one aspect, provided herein is an antibody that binds TRDV2, comprising a heavy chain having an amino acid sequence of SEQ ID NO:3467. In one aspect, provided herein is an antibody that binds TRDV2, comprising a light chain having an amino acid sequence of SEQ ID NO:3468. In one aspect, provided herein is an antibody that binds TRDV2, comprising a heavy chain having an amino acid sequence of SEQ ID NO:3467, and a light chain having an amino acid sequence of SEQ ID NO:3468. In one aspect, provided herein is an antibody that binds TRDV2, comprising a VH having an amino acid sequence having at least 95% identity to an amino acid sequence of SEQ ID NO:3465. In one aspect, provided herein is an antibody that binds TRDV2, comprising a VL having an amino acid sequence having at least 95% identity to an amino acid sequence of SEQ ID NO:3466. In one aspect, provided herein is an antibody that binds TRDV2, comprising a VH having an amino acid sequence having at least 95% identity to an amino acid sequence of SEQ ID NO:3465, and a VL having an amino acid sequence having at least 95% identity to an amino acid sequence of SEQ ID NO:3466. In one aspect, provided herein is an antibody that binds TRDV2, comprising a heavy chain having an amino acid sequence having at least 95% identity to an amino acid sequence of SEQ ID NO:3467. In one aspect, provided herein is an antibody that binds TRDV2, comprising a light chain having an amino acid sequence having at least 95% identity to an amino acid sequence of SEQ ID NO:3468. In one aspect, provided herein is an antibody that binds TRDV2, comprising a heavy chain having an amino acid sequence having at least 95% identity to an amino acid sequence of SEQ ID NO:3467, and a light chain having an amino acid sequence having at least 95% identity to an amino acid sequence of SEQ ID NO:3468.

In one aspect, provided herein is an antibody that binds TRDV2, comprising: (i) a VH comprising a VH CDR1, a VH CDR2, and a VH CDR3 having an amino acid sequence of SEQ ID NOs:3469, 3470, and 3471, respectively, and (ii) a VL comprising a VL CDR1, VL CDR2, and VL CDR3 having an amino acid sequence of SEQ ID NOs:3472, 3473, and 3474, respectively. In one aspect, provided herein is an antibody that binds TRDV2, comprising: (i) a VH comprising a VH CDR1, a VH CDR2, and a VH CDR3 having an amino acid sequence of SEQ ID NOs:3475, 3476, and 3477, respectively, and (ii) a VL comprising a VL CDR1, VL CDR2, and VL CDR3 having an amino acid sequence of SEQ ID NOs:3478, 3479, and 3480, respectively. In one aspect, provided herein is an antibody that binds TRDV2, comprising: (i) a VH comprising a VH CDR1, a VH CDR2, and a VH CDR3 having an amino acid sequence of SEQ ID NOs:3481, 3482, and 3483, respectively, and (ii) a VL comprising a VL CDR1, VL CDR2, and VL CDR3 having an amino acid sequence of SEQ ID NOs:3484, 3485, and 3486, respectively. In one aspect, provided herein is an antibody that binds TRDV2, comprising: (i) a VH comprising a VH CDR1, a VH CDR2, and a VH CDR3 having an amino acid sequence of SEQ ID NOs:3487, 3488, and 3489, respectively, and (ii) a VL comprising a VL CDR1, VL CDR2, and VL CDR3 having an amino acid sequence of SEQ ID NOs:3490, 3491, and 3492, respectively. In one aspect, provided herein is an antibody that binds TRDV2, comprising: (i) a VH comprising a VH CDR1, a VH CDR2, and a VH CDR3 having an amino acid sequence of SEQ ID NOs:3493, 3494, and 3495, respectively, and (ii) a VL comprising a VL CDR1, VL CDR2, and VL CDR3 having an amino acid sequence of SEQ ID NOs:3496, 3497, and 3498, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3499; and (ii) a VL comprising a VL CDR1, a VL CDR2, and a VL CDR3 having an amino acid sequence of a VL CDR1, a VL CDR2, and a VL CDR3, respectively, of SEQ ID NO:3500. In one aspect, provided herein is an antibody that binds TRDV2, comprising a VH having an amino acid sequence of SEQ ID NO:3499. In one aspect, provided herein is an antibody that binds TRDV2, comprising a VL having an amino acid sequence of SEQ ID NO:3500. In one aspect, provided herein is an antibody that binds TRDV2, comprising a VH having an amino acid sequence of SEQ ID NO:3499, and a VL having an amino acid sequence of SEQ ID NO:3500. In one aspect, provided herein is an antibody that binds TRDV2, comprising a heavy chain having an amino acid sequence of SEQ ID NO:3501. In one aspect, provided herein is an antibody that binds TRDV2, comprising a light chain having an amino acid sequence of SEQ ID NO:3502. In one aspect, provided herein is an antibody that binds TRDV2, comprising a heavy chain having an amino acid sequence of SEQ ID NO:3501, and a light chain having an amino acid sequence of SEQ ID NO:3502. In one aspect, provided herein is an antibody that binds TRDV2, comprising a VH having an amino acid sequence having at least 95% identity to an amino acid sequence of SEQ ID NO:3499. In one aspect, provided herein is an antibody that binds TRDV2, comprising a VL having an amino acid sequence having at least 95% identity to an amino acid sequence of SEQ ID NO:3500. In one aspect, provided herein is an antibody that binds TRDV2, comprising a VH having an amino acid sequence having at least 95% identity to an amino acid sequence of SEQ ID NO:3499, and a VL having an amino acid sequence having at least 95% identity to an amino acid sequence of SEQ ID NO:3500. In one aspect, provided herein is an antibody that binds TRDV2, comprising a heavy chain having an amino acid sequence having at least 95% identity to an amino acid sequence of SEQ ID NO:3501. In one aspect, provided herein is an antibody that binds TRDV2, comprising a light chain having an amino acid sequence having at least 95% identity to an amino acid sequence of SEQ ID NO:3502. In one aspect, provided herein is an antibody that binds TRDV2, comprising a heavy chain having an amino acid sequence having at least 95% identity to an amino acid sequence of SEQ ID NO:3501, and a light chain having an amino acid sequence having at least 95% identity to an amino acid sequence of SEQ ID NO:3502.

In one aspect, provided herein is an antibody that binds TRDV2, comprising: (i) a VH comprising a VH CDR1, a VH CDR2, and a VH CDR3 having an amino acid sequence of SEQ ID NOs:3503, 3504, and 3505, respectively, and (ii) a VL comprising a VL CDR1, VL CDR2, and VL CDR3 having an amino acid sequence of SEQ ID NOs:3506, 3507, and 3508, respectively. In one aspect, provided herein is an antibody that binds TRDV2, comprising: (i) a VH comprising a VH CDR1, a VH CDR2, and a VH CDR3 having an amino acid sequence of SEQ ID NOs:3509, 3510, and 3511, respectively, and (ii) a VL comprising a VL CDR1, VL CDR2, and VL CDR3 having an amino acid sequence of SEQ ID NOs:3512, 3513, and 3514, respectively. In one aspect, provided herein is an antibody that binds TRDV2, comprising: (i) a VH comprising a VH CDR1, a VH CDR2, and a VH CDR3 having an amino acid sequence of SEQ ID NOs:3515, 3516, and 3517, respectively, and (ii) a VL comprising a VL CDR1, VL CDR2, and VL CDR3 having an amino acid sequence of SEQ ID NOs:3518, 3519, and 3520, respectively. In one aspect, provided herein is an antibody that binds TRDV2, comprising: (i) a VH comprising a VH CDR1, a VH CDR2, and a VH CDR3 having an amino acid sequence of SEQ ID NOs:3521, 3522, and 3523, respectively, and (ii) a VL comprising a VL CDR1, VL CDR2, and VL CDR3 having an amino acid sequence of SEQ ID NOs:3524, 3525, and 3526, respectively. In one aspect, provided herein is an antibody that binds TRDV2, comprising: (i) a VH comprising a VH CDR1, a VH CDR2, and a VH CDR3 having an amino acid sequence of SEQ ID NOs:3527, 3528, and 3529, respectively, and (ii) a VL comprising a VL CDR1, VL CDR2, and VL CDR3 having an amino acid sequence of SEQ ID NOs:3530, 3531, and 3532, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3533; and (ii) a VL comprising a VL CDR1, a VL CDR2, and a VL CDR3 having an amino acid sequence of a VL CDR1, a VL CDR2, and a VL CDR3, respectively, of SEQ ID NO:3534. In one aspect, provided herein is an antibody that binds TRDV2, comprising a VH having an amino acid sequence of SEQ ID NO:3533. In one aspect, provided herein is an antibody that binds TRDV2, comprising a VL having an amino acid sequence of SEQ ID NO:3534. In one aspect, provided herein is an antibody that binds TRDV2, comprising a VH having an amino acid sequence of SEQ ID NO:3533, and a VL having an amino acid sequence of SEQ ID NO:3534. In one aspect, provided herein is an antibody that binds TRDV2, comprising a heavy chain having an amino acid sequence of SEQ ID NO:3535. In one aspect, provided herein is an antibody that binds TRDV2, comprising a light chain having an amino acid sequence of SEQ ID NO:3536. In one aspect, provided herein is an antibody that binds TRDV2, comprising a heavy chain having an amino acid sequence of SEQ ID NO:3535, and a light chain having an amino acid sequence of SEQ ID NO:3536. In one aspect, provided herein is an antibody that binds TRDV2, comprising a VH having an amino acid sequence having at least 95% identity to an amino acid sequence of SEQ ID NO:3533. In one aspect, provided herein is an antibody that binds TRDV2, comprising a VL having an amino acid sequence having at least 95% identity to an amino acid sequence of SEQ ID NO:3534. In one aspect, provided herein is an antibody that binds TRDV2, comprising a VH having an amino acid sequence having at least 95% identity to an amino acid sequence of SEQ ID NO:3533, and a VL having an amino acid sequence having at least 95% identity to an amino acid sequence of SEQ ID NO:3534. In one aspect, provided herein is an antibody that binds TRDV2, comprising a heavy chain having an amino acid sequence having at least 95% identity to an amino acid sequence of SEQ ID NO:3535. In one aspect, provided herein is an antibody that binds TRDV2, comprising a light chain having an amino acid sequence having at least 95% identity to an amino acid sequence of SEQ ID NO:3536. In one aspect, provided herein is an antibody that binds TRDV2, comprising a heavy chain having an amino acid sequence having at least 95% identity to an amino acid sequence of SEQ ID NO:3535, and a light chain having an amino acid sequence having at least 95% identity to an amino acid sequence of SEQ ID NO:3536.

In one aspect, provided herein is an antibody that binds TRDV2, comprising: (i) a VH comprising a VH CDR1, a VH CDR2, and a VH CDR3 having an amino acid sequence of SEQ ID NOs:3537, 3538, and 3539, respectively, and (ii) a VL comprising a VL CDR1, VL CDR2, and VL CDR3 having an amino acid sequence of SEQ ID NOs:3540, 3541, and 3542, respectively. In one aspect, provided herein is an antibody that binds TRDV2, comprising: (i) a VH comprising a VH CDR1, a VH CDR2, and a VH CDR3 having an amino acid sequence of SEQ ID NOs:3543, 3544, and 3545, respectively, and (ii) a VL comprising a VL CDR1, VL CDR2, and VL CDR3 having an amino acid sequence of SEQ ID NOs:3546, 3547, and 3548, respectively. In one aspect, provided herein is an antibody that binds TRDV2, comprising: (i) a VH comprising a VH CDR1, a VH CDR2, and a VH CDR3 having an amino acid sequence of SEQ ID NOs:3549, 3550, and 3551, respectively, and (ii) a VL comprising a VL CDR1, VL CDR2, and VL CDR3 having an amino acid sequence of SEQ ID NOs:3552, 3553, and 3554, respectively. In one aspect, provided herein is an antibody that binds TRDV2, comprising: (i) a VH comprising a VH CDR1, a VH CDR2, and a VH CDR3 having an amino acid sequence of SEQ ID NOs:3555, 3556, and 3557, respectively, and (ii) a VL comprising a VL CDR1, VL CDR2, and VL CDR3 having an amino acid sequence of SEQ ID NOs:3558, 3559, and 3560, respectively. In one aspect, provided herein is an antibody that binds TRDV2, comprising: (i) a VH comprising a VH CDR1, a VH CDR2, and a VH CDR3 having an amino acid sequence of SEQ ID NOs:3561, 3562, and 3563, respectively, and (ii) a VL comprising a VL CDR1, VL CDR2, and VL CDR3 having an amino acid sequence of SEQ ID NOs:3564, 3565, and 3566, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3567; and (ii) a VL comprising a VL CDR1, a VL CDR2, and a VL CDR3 having an amino acid sequence of a VL CDR1, a VL CDR2, and a VL CDR3, respectively, of SEQ ID NO:3568. In one aspect, provided herein is an antibody that binds TRDV2, comprising a VH having an amino acid sequence of SEQ ID NO:3567. In one aspect, provided herein is an antibody that binds TRDV2, comprising a VL having an amino acid sequence of SEQ ID NO:3568. In one aspect, provided herein is an antibody that binds TRDV2, comprising a VH having an amino acid sequence of SEQ ID NO:3567, and a VL having an amino acid sequence of SEQ ID NO:3568. In one aspect, provided herein is an antibody that binds TRDV2, comprising a heavy chain having an amino acid sequence of SEQ ID NO:3569. In one aspect, provided herein is an antibody that binds TRDV2, comprising a light chain having an amino acid sequence of SEQ ID NO:3570. In one aspect, provided herein is an antibody that binds TRDV2, comprising a heavy chain having an amino acid sequence of SEQ ID NO:3569, and a light chain having an amino acid sequence of SEQ ID NO:3570. In one aspect, provided herein is an antibody that binds TRDV2, comprising a VH having an amino acid sequence having at least 95% identity to an amino acid sequence of SEQ ID NO:3567. In one aspect, provided herein is an antibody that binds TRDV2, comprising a VL having an amino acid sequence having at least 95% identity to an amino acid sequence of SEQ ID NO:3568. In one aspect, provided herein is an antibody that binds TRDV2, comprising a VH having an amino acid sequence having at least 95% identity to an amino acid sequence of SEQ ID NO:3567, and a VL having an amino acid sequence having at least 95% identity to an amino acid sequence of SEQ ID NO:3568. In one aspect, provided herein is an antibody that binds TRDV2, comprising a heavy chain having an amino acid sequence having at least 95% identity to an amino acid sequence of SEQ ID NO:3569. In one aspect, provided herein is an antibody that binds TRDV2, comprising a light chain having an amino acid sequence having at least 95% identity to an amino acid sequence of SEQ ID NO:3570. In one aspect, provided herein is an antibody that binds TRDV2, comprising a heavy chain having an amino acid sequence having at least 95% identity to an amino acid sequence of SEQ ID NO:3569, and a light chain having an amino acid sequence having at least 95% identity to an amino acid sequence of SEQ ID NO:3570.

In one aspect, provided herein is an antibody that binds TRDV2, comprising: (i) a VH comprising a VH CDR1, a VH CDR2, and a VH CDR3 having an amino acid sequence of SEQ ID NOs:3571, 3572, and 3573, respectively, and (ii) a VL comprising a VL CDR1, VL CDR2, and VL CDR3 having an amino acid sequence of SEQ ID NOs:3574, 3575, and 3576, respectively. In one aspect, provided herein is an antibody that binds TRDV2, comprising: (i) a VH comprising a VH CDR1, a VH CDR2, and a VH CDR3 having an amino acid sequence of SEQ ID NOs:3577, 3578, and 3579, respectively, and (ii) a VL comprising a VL CDR1, VL CDR2, and VL CDR3 having an amino acid sequence of SEQ ID NOs:3580, 3581, and 3582, respectively. In one aspect, provided herein is an antibody that binds TRDV2, comprising: (i) a VH comprising a VH CDR1, a VH CDR2, and a VH CDR3 having an amino acid sequence of SEQ ID NOs:3583, 3584, and 3585, respectively, and (ii) a VL comprising a VL CDR1, VL CDR2, and VL CDR3 having an amino acid sequence of SEQ ID NOs:3586, 3587, and 3588, respectively. In one aspect, provided herein is an antibody that binds TRDV2, comprising: (i) a VH comprising a VH CDR1, a VH CDR2, and a VH CDR3 having an amino acid sequence of SEQ ID NOs:3589, 3590, and 3591, respectively, and (ii) a VL comprising a VL CDR1, VL CDR2, and VL CDR3 having an amino acid sequence of SEQ ID NOs:3592, 3593, and 3594, respectively. In one aspect, provided herein is an antibody that binds TRDV2, comprising: (i) a VH comprising a VH CDR1, a VH CDR2, and a VH CDR3 having an amino acid sequence of SEQ ID NOs:3595, 3596, and 3597, respectively, and (ii) a VL comprising a VL CDR1, VL CDR2, and VL CDR3 having an amino acid sequence of SEQ ID NOs:3598, 3599, and 3600, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3601; and (ii) a VL comprising a VL CDR1, a VL CDR2, and a VL CDR3 having an amino acid sequence of a VL CDR1, a VL CDR2, and a VL CDR3, respectively, of SEQ ID NO:3602. In one aspect, provided herein is an antibody that binds TRDV2, comprising a VH having an amino acid sequence of SEQ ID NO:3601. In one aspect, provided herein is an antibody that binds TRDV2, comprising a VL having an amino acid sequence of SEQ ID NO:3602. In one aspect, provided herein is an antibody that binds TRDV2, comprising a VH having an amino acid sequence of SEQ ID NO:3601, and a VL having an amino acid sequence of SEQ ID NO:3602. In one aspect, provided herein is an antibody that binds TRDV2, comprising a heavy chain having an amino acid sequence of SEQ ID NO:3603. In one aspect, provided herein is an antibody that binds TRDV2, comprising a light chain having an amino acid sequence of SEQ ID NO:3604. In one aspect, provided herein is an antibody that binds TRDV2, comprising a heavy chain having an amino acid sequence of SEQ ID NO:3603, and a light chain having an amino acid sequence of SEQ ID NO:3604. In one aspect, provided herein is an antibody that binds TRDV2, comprising a VH having an amino acid sequence having at least 95% identity to an amino acid sequence of SEQ ID NO:3601. In one aspect, provided herein is an antibody that binds TRDV2, comprising a VL having an amino acid sequence having at least 95% identity to an amino acid sequence of SEQ ID NO:3602. In one aspect, provided herein is an antibody that binds TRDV2, comprising a VH having an amino acid sequence having at least 95% identity to an amino acid sequence of SEQ ID NO:3601, and a VL having an amino acid sequence having at least 95% identity to an amino acid sequence of SEQ ID NO:3602. In one aspect, provided herein is an antibody that binds TRDV2, comprising a heavy chain having an amino acid sequence having at least 95% identity to an amino acid sequence of SEQ ID NO:3603. In one aspect, provided herein is an antibody that binds TRDV2, comprising a light chain having an amino acid sequence having at least 95% identity to an amino acid sequence of SEQ ID NO:3604. In one aspect, provided herein is an antibody that binds TRDV2, comprising a heavy chain having an amino acid sequence having at least 95% identity to an amino acid sequence of SEQ ID NO:3603, and a light chain having an amino acid sequence having at least 95% identity to an amino acid sequence of SEQ ID NO:3604.

In one aspect, provided herein is an antibody that binds TRDV2, comprising: (i) a VH comprising a VH CDR1, a VH CDR2, and a VH CDR3 having an amino acid sequence of SEQ ID NOs:3605, 3606, and 3607, respectively, and (ii) a VL comprising a VL CDR1, VL CDR2, and VL CDR3 having an amino acid sequence of SEQ ID NOs:3608, 3609, and 3610, respectively. In one aspect, provided herein is an antibody that binds TRDV2, comprising: (i) a VH comprising a VH CDR1, a VH CDR2, and a VH CDR3 having an amino acid sequence of SEQ ID NOs:3611, 3612, and 3613, respectively, and (ii) a VL comprising a VL CDR1, VL CDR2, and VL CDR3 having an amino acid sequence of SEQ ID NOs:3614, 3615, and 3616, respectively. In one aspect, provided herein is an antibody that binds TRDV2, comprising: (i) a VH comprising a VH CDR1, a VH CDR2, and a VH CDR3 having an amino acid sequence of SEQ ID NOs:3617, 3618, and 3619, respectively, and (ii) a VL comprising a VL CDR1, VL CDR2, and VL CDR3 having an amino acid sequence of SEQ ID NOs:3620, 3621, and 3622, respectively. In one aspect, provided herein is an antibody that binds TRDV2, comprising: (i) a VH comprising a VH CDR1, a VH CDR2, and a VH CDR3 having an amino acid sequence of SEQ ID NOs:3623, 3624, and 3625, respectively, and (ii) a VL comprising a VL CDR1, VL CDR2, and VL CDR3 having an amino acid sequence of SEQ ID NOs:3626, 3627, and 3628, respectively. In one aspect, provided herein is an antibody that binds TRDV2, comprising: (i) a VH comprising a VH CDR1, a VH CDR2, and a VH CDR3 having an amino acid sequence of SEQ ID NOs:3629, 3630, and 3631, respectively, and (ii) a VL comprising a VL CDR1, VL CDR2, and VL CDR3 having an amino acid sequence of SEQ ID NOs:3632, 3633, and 3634, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3635; and (ii) a VL comprising a VL CDR1, a VL CDR2, and a VL CDR3 having an amino acid sequence of a VL CDR1, a VL CDR2, and a VL CDR3, respectively, of SEQ ID NO:3636. In one aspect, provided herein is an antibody that binds TRDV2, comprising a VH having an amino acid sequence of SEQ ID NO:3635. In one aspect, provided herein is an antibody that binds TRDV2, comprising a VL having an amino acid sequence of SEQ ID NO:3636. In one aspect, provided herein is an antibody that binds TRDV2, comprising a VH having an amino acid sequence of SEQ ID NO:3635, and a VL having an amino acid sequence of SEQ ID NO:3636. In one aspect, provided herein is an antibody that binds TRDV2, comprising a heavy chain having an amino acid sequence of SEQ ID NO:3637. In one aspect, provided herein is an antibody that binds TRDV2, comprising a light chain having an amino acid sequence of SEQ ID NO:3638. In one aspect, provided herein is an antibody that binds TRDV2, comprising a heavy chain having an amino acid sequence of SEQ ID NO:3637, and a light chain having an amino acid sequence of SEQ ID NO:3638. In one aspect, provided herein is an antibody that binds TRDV2, comprising a VH having an amino acid sequence having at least 95% identity to an amino acid sequence of SEQ ID NO:3635. In one aspect, provided herein is an antibody that binds TRDV2, comprising a VL having an amino acid sequence having at least 95% identity to an amino acid sequence of SEQ ID NO:3636. In one aspect, provided herein is an antibody that binds TRDV2, comprising a VH having an amino acid sequence having at least 95% identity to an amino acid sequence of SEQ ID NO:3635, and a VL having an amino acid sequence having at least 95% identity to an amino acid sequence of SEQ ID NO:3636. In one aspect, provided herein is an antibody that binds TRDV2, comprising a heavy chain having an amino acid sequence having at least 95% identity to an amino acid sequence of SEQ ID NO:3637. In one aspect, provided herein is an antibody that binds TRDV2, comprising a light chain having an amino acid sequence having at least 95% identity to an amino acid sequence of SEQ ID NO:3638. In one aspect, provided herein is an antibody that binds TRDV2, comprising a heavy chain having an amino acid sequence having at least 95% identity to an amino acid sequence of SEQ ID NO:3637, and a light chain having an amino acid sequence having at least 95% identity to an amino acid sequence of SEQ ID NO:3638.

In one aspect, provided herein is an antibody that binds TRDV2, comprising: (i) a VH comprising a VH CDR1, a VH CDR2, and a VH CDR3 having an amino acid sequence of SEQ ID NOs:3639, 3640, and 3641, respectively, and (ii) a VL comprising a VL CDR1, VL CDR2, and VL CDR3 having an amino acid sequence of SEQ ID NOs:3642, 3643, and 3644, respectively. In one aspect, provided herein is an antibody that binds TRDV2, comprising: (i) a VH comprising a VH CDR1, a VH CDR2, and a VH CDR3 having an amino acid sequence of SEQ ID NOs:3645, 3646, and 3647, respectively, and (ii) a VL comprising a VL CDR1, VL CDR2, and VL CDR3 having an amino acid sequence of SEQ ID NOs:3648, 3649, and 3650, respectively. In one aspect, provided herein is an antibody that binds TRDV2, comprising: (i) a VH comprising a VH CDR1, a VH CDR2, and a VH CDR3 having an amino acid sequence of SEQ ID NOs:3651, 3652, and 3653, respectively, and (ii) a VL comprising a VL CDR1, VL CDR2, and VL CDR3 having an amino acid sequence of SEQ ID NOs:3654, 3655, and 3656, respectively. In one aspect, provided herein is an antibody that binds TRDV2, comprising: (i) a VH comprising a VH CDR1, a VH CDR2, and a VH CDR3 having an amino acid sequence of SEQ ID NOs:3657, 3658, and 3659, respectively, and (ii) a VL comprising a VL CDR1, VL CDR2, and VL CDR3 having an amino acid sequence of SEQ ID NOs:3660, 3661, and 3662, respectively. In one aspect, provided herein is an antibody that binds TRDV2, comprising: (i) a VH comprising a VH CDR1, a VH CDR2, and a VH CDR3 having an amino acid sequence of SEQ ID NOs:3663, 3664, and 3665, respectively, and (ii) a VL comprising a VL CDR1, VL CDR2, and VL CDR3 having an amino acid sequence of SEQ ID NOs:3666, 3667, and 3668, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3669; and (ii) a VL comprising a VL CDR1, a VL CDR2, and a VL CDR3 having an amino acid sequence of a VL CDR1, a VL CDR2, and a VL CDR3, respectively, of SEQ ID NO:3670. In one aspect, provided herein is an antibody that binds TRDV2, comprising a VH having an amino acid sequence of SEQ ID NO:3669. In one aspect, provided herein is an antibody that binds TRDV2, comprising a VL having an amino acid sequence of SEQ ID NO:3670. In one aspect, provided herein is an antibody that binds TRDV2, comprising a VH having an amino acid sequence of SEQ ID NO:3669, and a VL having an amino acid sequence of SEQ ID NO:3670. In one aspect, provided herein is an antibody that binds TRDV2, comprising a heavy chain having an amino acid sequence of SEQ ID NO:3671. In one aspect, provided herein is an antibody that binds TRDV2, comprising a light chain having an amino acid sequence of SEQ ID NO:3672. In one aspect, provided herein is an antibody that binds TRDV2, comprising a heavy chain having an amino acid sequence of SEQ ID NO:3671, and a light chain having an amino acid sequence of SEQ ID NO:3672. In one aspect, provided herein is an antibody that binds TRDV2, comprising a VH having an amino acid sequence having at least 95% identity to an amino acid sequence of SEQ ID NO:3669. In one aspect, provided herein is an antibody that binds TRDV2, comprising a VL having an amino acid sequence having at least 95% identity to an amino acid sequence of SEQ ID NO:3670. In one aspect, provided herein is an antibody that binds TRDV2, comprising a VH having an amino acid sequence having at least 95% identity to an amino acid sequence of SEQ ID NO:3669, and a VL having an amino acid sequence having at least 95% identity to an amino acid sequence of SEQ ID NO:3670. In one aspect, provided herein is an antibody that binds TRDV2, comprising a heavy chain having an amino acid sequence having at least 95% identity to an amino acid sequence of SEQ ID NO:3671. In one aspect, provided herein is an antibody that binds TRDV2, comprising a light chain having an amino acid sequence having at least 95% identity to an amino acid sequence of SEQ ID NO:3672. In one aspect, provided herein is an antibody that binds TRDV2, comprising a heavy chain having an amino acid sequence having at least 95% identity to an amino acid sequence of SEQ ID NO:3671, and a light chain having an amino acid sequence having at least 95% identity to an amino acid sequence of SEQ ID NO:3672.

In one aspect, provided herein is an antibody that binds TRDV2, comprising: (i) a VH comprising a VH CDR1, a VH CDR2, and a VH CDR3 having an amino acid sequence of SEQ ID NOs:3673, 3674, and 3675, respectively, and (ii) a VL comprising a VL CDR1, VL CDR2, and VL CDR3 having an amino acid sequence of SEQ ID NOs:3676, 3677, and 3678, respectively. In one aspect, provided herein is an antibody that binds TRDV2, comprising: (i) a VH comprising a VH CDR1, a VH CDR2, and a VH CDR3 having an amino acid sequence of SEQ ID NOs:3679, 3680, and 3681, respectively, and (ii) a VL comprising a VL CDR1, VL CDR2, and VL CDR3 having an amino acid sequence of SEQ ID NOs:3682, 3683, and 3684, respectively. In one aspect, provided herein is an antibody that binds TRDV2, comprising: (i) a VH comprising a VH CDR1, a VH CDR2, and a VH CDR3 having an amino acid sequence of SEQ ID NOs:3685, 3686, and 3687, respectively, and (ii) a VL comprising a VL CDR1, VL CDR2, and VL CDR3 having an amino acid sequence of SEQ ID NOs:3688, 3689, and 3690, respectively. In one aspect, provided herein is an antibody that binds TRDV2, comprising: (i) a VH comprising a VH CDR1, a VH CDR2, and a VH CDR3 having an amino acid sequence of SEQ ID NOs:3691, 3692, and 3693, respectively, and (ii) a VL comprising a VL CDR1, VL CDR2, and VL CDR3 having an amino acid sequence of SEQ ID NOs:3694, 3695, and 3696, respectively. In one aspect, provided herein is an antibody that binds TRDV2, comprising: (i) a VH comprising a VH CDR1, a VH CDR2, and a VH CDR3 having an amino acid sequence of SEQ ID NOs:3697, 3698, and 3699, respectively, and (ii) a VL comprising a VL CDR1, VL CDR2, and VL CDR3 having an amino acid sequence of SEQ ID NOs:3700, 3701, and 3702, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3703; and (ii) a VL comprising a VL CDR1, a VL CDR2, and a VL CDR3 having an amino acid sequence of a VL CDR1, a VL CDR2, and a VL CDR3, respectively, of SEQ ID NO:3704. In one aspect, provided herein is an antibody that binds TRDV2, comprising a VH having an amino acid sequence of SEQ ID NO:3703. In one aspect, provided herein is an antibody that binds TRDV2, comprising a VL having an amino acid sequence of SEQ ID NO:3704. In one aspect, provided herein is an antibody that binds TRDV2, comprising a VH having an amino acid sequence of SEQ ID NO:3703, and a VL having an amino acid sequence of SEQ ID NO:3704. In one aspect, provided herein is an antibody that binds TRDV2, comprising a heavy chain having an amino acid sequence of SEQ ID NO:3705. In one aspect, provided herein is an antibody that binds TRDV2, comprising a light chain having an amino acid sequence of SEQ ID NO:3706. In one aspect, provided herein is an antibody that binds TRDV2, comprising a heavy chain having an amino acid sequence of SEQ ID NO:3705, and a light chain having an amino acid sequence of SEQ ID NO:3706. In one aspect, provided herein is an antibody that binds TRDV2, comprising a VH having an amino acid sequence having at least 95% identity to an amino acid sequence of SEQ ID NO:3703. In one aspect, provided herein is an antibody that binds TRDV2, comprising a VL having an amino acid sequence having at least 95% identity to an amino acid sequence of SEQ ID NO:3704. In one aspect, provided herein is an antibody that binds TRDV2, comprising a VH having an amino acid sequence having at least 95% identity to an amino acid sequence of SEQ ID NO:3703, and a VL having an amino acid sequence having at least 95% identity to an amino acid sequence of SEQ ID NO:3704. In one aspect, provided herein is an antibody that binds TRDV2, comprising a heavy chain having an amino acid sequence having at least 95% identity to an amino acid sequence of SEQ ID NO:3705. In one aspect, provided herein is an antibody that binds TRDV2, comprising a light chain having an amino acid sequence having at least 95% identity to an amino acid sequence of SEQ ID NO:3706. In one aspect, provided herein is an antibody that binds TRDV2, comprising a heavy chain having an amino acid sequence having at least 95% identity to an amino acid sequence of SEQ ID NO:3705, and a light chain having an amino acid sequence having at least 95% identity to an amino acid sequence of SEQ ID NO:3706.

In one aspect, provided herein is an antibody that binds TRDV2, comprising: (i) a VH comprising a VH CDR1, a VH CDR2, and a VH CDR3 having an amino acid sequence of SEQ ID NOs:3707, 3708, and 3709, respectively, and (ii) a VL comprising a VL CDR1, VL CDR2, and VL CDR3 having an amino acid sequence of SEQ ID NOs:3710, 3711, and 3712, respectively. In one aspect, provided herein is an antibody that binds TRDV2, comprising: (i) a VH comprising a VH CDR1, a VH CDR2, and a VH CDR3 having an amino acid sequence of SEQ ID NOs:3713, 3714, and 3715, respectively, and (ii) a VL comprising a VL CDR1, VL CDR2, and VL CDR3 having an amino acid sequence of SEQ ID NOs:3716, 3717, and 3718, respectively. In one aspect, provided herein is an antibody that binds TRDV2, comprising: (i) a VH comprising a VH CDR1, a VH CDR2, and a VH CDR3 having an amino acid sequence of SEQ ID NOs:3719, 3720, and 3721, respectively, and (ii) a VL comprising a VL CDR1, VL CDR2, and VL CDR3 having an amino acid sequence of SEQ ID NOs:3722, 3723, and 3724, respectively. In one aspect, provided herein is an antibody that binds TRDV2, comprising: (i) a VH comprising a VH CDR1, a VH CDR2, and a VH CDR3 having an amino acid sequence of SEQ ID NOs:3725, 3726, and 3727, respectively, and (ii) a VL comprising a VL CDR1, VL CDR2, and VL CDR3 having an amino acid sequence of SEQ ID NOs:3728, 3729, and 3730, respectively. In one aspect, provided herein is an antibody that binds TRDV2, comprising: (i) a VH comprising a VH CDR1, a VH CDR2, and a VH CDR3 having an amino acid sequence of SEQ ID NOs:3731, 3732, and 3733, respectively, and (ii) a VL comprising a VL CDR1, VL CDR2, and VL CDR3 having an amino acid sequence of SEQ ID NOs:3734, 3735, and 3736, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3737; and (ii) a VL comprising a VL CDR1, a VL CDR2, and a VL CDR3 having an amino acid sequence of a VL CDR1, a VL CDR2, and a VL CDR3, respectively, of SEQ ID NO:3738. In one aspect, provided herein is an antibody that binds TRDV2, comprising a VH having an amino acid sequence of SEQ ID NO:3737. In one aspect, provided herein is an antibody that binds TRDV2, comprising a VL having an amino acid sequence of SEQ ID NO:3738. In one aspect, provided herein is an antibody that binds TRDV2, comprising a VH having an amino acid sequence of SEQ ID NO:3737, and a VL having an amino acid sequence of SEQ ID NO:3738. In one aspect, provided herein is an antibody that binds TRDV2, comprising a heavy chain having an amino acid sequence of SEQ ID NO:3739. In one aspect, provided herein is an antibody that binds TRDV2, comprising a light chain having an amino acid sequence of SEQ ID NO:3740. In one aspect, provided herein is an antibody that binds TRDV2, comprising a heavy chain having an amino acid sequence of SEQ ID NO:3739, and a light chain having an amino acid sequence of SEQ ID NO:3740. In one aspect, provided herein is an antibody that binds TRDV2, comprising a VH having an amino acid sequence having at least 95% identity to an amino acid sequence of SEQ ID NO:3737. In one aspect, provided herein is an antibody that binds TRDV2, comprising a VL having an amino acid sequence having at least 95% identity to an amino acid sequence of SEQ ID NO:3738. In one aspect, provided herein is an antibody that binds TRDV2, comprising a VH having an amino acid sequence having at least 95% identity to an amino acid sequence of SEQ ID NO:3737, and a VL having an amino acid sequence having at least 95% identity to an amino acid sequence of SEQ ID NO:3738. In one aspect, provided herein is an antibody that binds TRDV2, comprising a heavy chain having an amino acid sequence having at least 95% identity to an amino acid sequence of SEQ ID NO:3739. In one aspect, provided herein is an antibody that binds TRDV2, comprising a light chain having an amino acid sequence having at least 95% identity to an amino acid sequence of SEQ ID NO:3740. In one aspect, provided herein is an antibody that binds TRDV2, comprising a heavy chain having an amino acid sequence having at least 95% identity to an amino acid sequence of SEQ ID NO:3739, and a light chain having an amino acid sequence having at least 95% identity to an amino acid sequence of SEQ ID NO:3740.

In one aspect, provided herein is an antibody that binds TRDV2, comprising: (i) a VH comprising a VH CDR1, a VH CDR2, and a VH CDR3 having an amino acid sequence of SEQ ID NOs:3741, 3742, and 3743, respectively, and (ii) a VL comprising a VL CDR1, VL CDR2, and VL CDR3 having an amino acid sequence of SEQ ID NOs:3744, 3745, and 3746, respectively. In one aspect, provided herein is an antibody that binds TRDV2, comprising: (i) a VH comprising a VH CDR1, a VH CDR2, and a VH CDR3 having an amino acid sequence of SEQ ID NOs:3747, 3748, and 3749, respectively, and (ii) a VL comprising a VL CDR1, VL CDR2, and VL CDR3 having an amino acid sequence of SEQ ID NOs:3750, 3751, and 3752, respectively. In one aspect, provided herein is an antibody that binds TRDV2, comprising: (i) a VH comprising a VH CDR1, a VH CDR2, and a VH CDR3 having an amino acid sequence of SEQ ID NOs:3753, 3754, and 3755, respectively, and (ii) a VL comprising a VL CDR1, VL CDR2, and VL CDR3 having an amino acid sequence of SEQ ID NOs:3756, 3757, and 3758, respectively. In one aspect, provided herein is an antibody that binds TRDV2, comprising: (i) a VH comprising a VH CDR1, a VH CDR2, and a VH CDR3 having an amino acid sequence of SEQ ID NOs:3759, 3760, and 3761, respectively, and (ii) a VL comprising a VL CDR1, VL CDR2, and VL CDR3 having an amino acid sequence of SEQ ID NOs:3762, 3763, and 3764, respectively. In one aspect, provided herein is an antibody that binds TRDV2, comprising: (i) a VH comprising a VH CDR1, a VH CDR2, and a VH CDR3 having an amino acid sequence of SEQ ID NOs:3765, 3766, and 3767, respectively, and (ii) a VL comprising a VL CDR1, VL CDR2, and VL CDR3 having an amino acid sequence of SEQ ID NOs:3768, 3769, and 3770, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3771; and (ii) a VL comprising a VL CDR1, a VL CDR2, and a VL CDR3 having an amino acid sequence of a VL CDR1, a VL CDR2, and a VL CDR3, respectively, of SEQ ID NO:3772. In one aspect, provided herein is an antibody that binds TRDV2, comprising a VH having an amino acid sequence of SEQ ID NO:3771. In one aspect, provided herein is an antibody that binds TRDV2, comprising a VL having an amino acid sequence of SEQ ID NO:3772. In one aspect, provided herein is an antibody that binds TRDV2, comprising a VH having an amino acid sequence of SEQ ID NO:3771, and a VL having an amino acid sequence of SEQ ID NO:3772. In one aspect, provided herein is an antibody that binds TRDV2, comprising a heavy chain having an amino acid sequence of SEQ ID NO:3773. In one aspect, provided herein is an antibody that binds TRDV2, comprising a light chain having an amino acid sequence of SEQ ID NO:3774. In one aspect, provided herein is an antibody that binds TRDV2, comprising a heavy chain having an amino acid sequence of SEQ ID NO:3773, and a light chain having an amino acid sequence of SEQ ID NO:3774. In one aspect, provided herein is an antibody that binds TRDV2, comprising a VH having an amino acid sequence having at least 95% identity to an amino acid sequence of SEQ ID NO:3771. In one aspect, provided herein is an antibody that binds TRDV2, comprising a VL having an amino acid sequence having at least 95% identity to an amino acid sequence of SEQ ID NO:3772. In one aspect, provided herein is an antibody that binds TRDV2, comprising a VH having an amino acid sequence having at least 95% identity to an amino acid sequence of SEQ ID NO:3771, and a VL having an amino acid sequence having at least 95% identity to an amino acid sequence of SEQ ID NO:3772. In one aspect, provided herein is an antibody that binds TRDV2, comprising a heavy chain having an amino acid sequence having at least 95% identity to an amino acid sequence of SEQ ID NO:3773. In one aspect, provided herein is an antibody that binds TRDV2, comprising a light chain having an amino acid sequence having at least 95% identity to an amino acid sequence of SEQ ID NO:3774. In one aspect, provided herein is an antibody that binds TRDV2, comprising a heavy chain having an amino acid sequence having at least 95% identity to an amino acid sequence of SEQ ID NO:3773, and a light chain having an amino acid sequence having at least 95% identity to an amino acid sequence of SEQ ID NO:3774.

In one aspect, provided herein is an antibody that binds TRDV2, comprising: (i) a VH comprising a VH CDR1, a VH CDR2, and a VH CDR3 having an amino acid sequence of SEQ ID NOs:3775, 3776, and 3777, respectively, and (ii) a VL comprising a VL CDR1, VL CDR2, and VL CDR3 having an amino acid sequence of SEQ ID NOs:3778, 3779, and 3780, respectively. In one aspect, provided herein is an antibody that binds TRDV2, comprising: (i) a VH comprising a VH CDR1, a VH CDR2, and a VH CDR3 having an amino acid sequence of SEQ ID NOs:3781, 3782, and 3783, respectively, and (ii) a VL comprising a VL CDR1, VL CDR2, and VL CDR3 having an amino acid sequence of SEQ ID NOs:3784, 3785, and 3786, respectively. In one aspect, provided herein is an antibody that binds TRDV2, comprising: (i) a VH comprising a VH CDR1, a VH CDR2, and a VH CDR3 having an amino acid sequence of SEQ ID NOs:3787, 3788, and 3789, respectively, and (ii) a VL comprising a VL CDR1, VL CDR2, and VL CDR3 having an amino acid sequence of SEQ ID NOs:3790, 3791, and 3792, respectively. In one aspect, provided herein is an antibody that binds TRDV2, comprising: (i) a VH comprising a VH CDR1, a VH CDR2, and a VH CDR3 having an amino acid sequence of SEQ ID NOs:3793, 3794, and 3795, respectively, and (ii) a VL comprising a VL CDR1, VL CDR2, and VL CDR3 having an amino acid sequence of SEQ ID NOs:3796, 3797, and 3798, respectively. In one aspect, provided herein is an antibody that binds TRDV2, comprising: (i) a VH comprising a VH CDR1, a VH CDR2, and a VH CDR3 having an amino acid sequence of SEQ ID NOs:3799, 3800, and 3801, respectively, and (ii) a VL comprising a VL CDR1, VL CDR2, and VL CDR3 having an amino acid sequence of SEQ ID NOs:3802, 3803, and 3804, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3805; and (ii) a VL comprising a VL CDR1, a VL CDR2, and a VL CDR3 having an amino acid sequence of a VL CDR1, a VL CDR2, and a VL CDR3, respectively, of SEQ ID NO:3806. In one aspect, provided herein is an antibody that binds TRDV2, comprising a VH having an amino acid sequence of SEQ ID NO:3805. In one aspect, provided herein is an antibody that binds TRDV2, comprising a VL having an amino acid sequence of SEQ ID NO:3806. In one aspect, provided herein is an antibody that binds TRDV2, comprising a VH having an amino acid sequence of SEQ ID NO:3805, and a VL having an amino acid sequence of SEQ ID NO:3806. In one aspect, provided herein is an antibody that binds TRDV2, comprising a heavy chain having an amino acid sequence of SEQ ID NO:3807. In one aspect, provided herein is an antibody that binds TRDV2, comprising a light chain having an amino acid sequence of SEQ ID NO:3808. In one aspect, provided herein is an antibody that binds TRDV2, comprising a heavy chain having an amino acid sequence of SEQ ID NO:3807, and a light chain having an amino acid sequence of SEQ ID NO:3808. In one aspect, provided herein is an antibody that binds TRDV2, comprising a VH having an amino acid sequence having at least 95% identity to an amino acid sequence of SEQ ID NO:3805. In one aspect, provided herein is an antibody that binds TRDV2, comprising a VL having an amino acid sequence having at least 95% identity to an amino acid sequence of SEQ ID NO:3806. In one aspect, provided herein is an antibody that binds TRDV2, comprising a VH having an amino acid sequence having at least 95% identity to an amino acid sequence of SEQ ID NO:3805, and a VL having an amino acid sequence having at least 95% identity to an amino acid sequence of SEQ ID NO:3806. In one aspect, provided herein is an antibody that binds TRDV2, comprising a heavy chain having an amino acid sequence having at least 95% identity to an amino acid sequence of SEQ ID NO:3807. In one aspect, provided herein is an antibody that binds TRDV2, comprising a light chain having an amino acid sequence having at least 95% identity to an amino acid sequence of SEQ ID NO:3808. In one aspect, provided herein is an antibody that binds TRDV2, comprising a heavy chain having an amino acid sequence having at least 95% identity to an amino acid sequence of SEQ ID NO:3807, and a light chain having an amino acid sequence having at least 95% identity to an amino acid sequence of SEQ ID NO:3808.

In one aspect, provided herein is an antibody that binds TRDV2, comprising: (i) a VH comprising a VH CDR1, a VH CDR2, and a VH CDR3 having an amino acid sequence of SEQ ID NOs:3809, 3810, and 3811, respectively, and (ii) a VL comprising a VL CDR1, VL CDR2, and VL CDR3 having an amino acid sequence of SEQ ID NOs:3812, 3813, and 3814, respectively. In one aspect, provided herein is an antibody that binds TRDV2, comprising: (i) a VH comprising a VH CDR1, a VH CDR2, and a VH CDR3 having an amino acid sequence of SEQ ID NOs:3815, 3816, and 3817, respectively, and (ii) a VL comprising a VL CDR1, VL CDR2, and VL CDR3 having an amino acid sequence of SEQ ID NOs:3818, 3819, and 3820, respectively. In one aspect, provided herein is an antibody that binds TRDV2, comprising: (i) a VH comprising a VH CDR1, a VH CDR2, and a VH CDR3 having an amino acid sequence of SEQ ID NOs:3821, 3822, and 3823, respectively, and (ii) a VL comprising a VL CDR1, VL CDR2, and VL CDR3 having an amino acid sequence of SEQ ID NOs:3824, 3825, and 3826, respectively. In one aspect, provided herein is an antibody that binds TRDV2, comprising: (i) a VH comprising a VH CDR1, a VH CDR2, and a VH CDR3 having an amino acid sequence of SEQ ID NOs:3827, 3828, and 3829, respectively, and (ii) a VL comprising a VL CDR1, VL CDR2, and VL CDR3 having an amino acid sequence of SEQ ID NOs:3830, 3831, and 3832, respectively. In one aspect, provided herein is an antibody that binds TRDV2, comprising: (i) a VH comprising a VH CDR1, a VH CDR2, and a VH CDR3 having an amino acid sequence of SEQ ID NOs:3833, 3834, and 3835, respectively, and (ii) a VL comprising a VL CDR1, VL CDR2, and VL CDR3 having an amino acid sequence of SEQ ID NOs:3836, 3837, and 3838, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3839; and (ii) a VL comprising a VL CDR1, a VL CDR2, and a VL CDR3 having an amino acid sequence of a VL CDR1, a VL CDR2, and a VL CDR3, respectively, of SEQ ID NO:3840. In one aspect, provided herein is an antibody that binds TRDV2, comprising a VH having an amino acid sequence of SEQ ID NO:3839. In one aspect, provided herein is an antibody that binds TRDV2, comprising a VL having an amino acid sequence of SEQ ID NO:3840. In one aspect, provided herein is an antibody that binds TRDV2, comprising a VH having an amino acid sequence of SEQ ID NO:3839, and a VL having an amino acid sequence of SEQ ID NO:3840. In one aspect, provided herein is an antibody that binds TRDV2, comprising a heavy chain having an amino acid sequence of SEQ ID NO:3841. In one aspect, provided herein is an antibody that binds TRDV2, comprising a light chain having an amino acid sequence of SEQ ID NO:3842. In one aspect, provided herein is an antibody that binds TRDV2, comprising a heavy chain having an amino acid sequence of SEQ ID NO:3841, and a light chain having an amino acid sequence of SEQ ID NO:3842. In one aspect, provided herein is an antibody that binds TRDV2, comprising a VH having an amino acid sequence having at least 95% identity to an amino acid sequence of SEQ ID NO:3839. In one aspect, provided herein is an antibody that binds TRDV2, comprising a VL having an amino acid sequence having at least 95% identity to an amino acid sequence of SEQ ID NO:3840. In one aspect, provided herein is an antibody that binds TRDV2, comprising a VH having an amino acid sequence having at least 95% identity to an amino acid sequence of SEQ ID NO:3839, and a VL having an amino acid sequence having at least 95% identity to an amino acid sequence of SEQ ID NO:3840. In one aspect, provided herein is an antibody that binds TRDV2, comprising a heavy chain having an amino acid sequence having at least 95% identity to an amino acid sequence of SEQ ID NO:3841. In one aspect, provided herein is an antibody that binds TRDV2, comprising a light chain having an amino acid sequence having at least 95% identity to an amino acid sequence of SEQ ID NO:3842. In one aspect, provided herein is an antibody that binds TRDV2, comprising a heavy chain having an amino acid sequence having at least 95% identity to an amino acid sequence of SEQ ID NO:3841, and a light chain having an amino acid sequence having at least 95% identity to an amino acid sequence of SEQ ID NO:3842.

In one aspect, provided herein is an antibody that binds TRDV2, comprising: (i) a VH comprising a VH CDR1, a VH CDR2, and a VH CDR3 having an amino acid sequence of SEQ ID NOs:3843, 3844, and 3845, respectively, and (ii) a VL comprising a VL CDR1, VL CDR2, and VL CDR3 having an amino acid sequence of SEQ ID NOs:3846, 3847, and 3848, respectively. In one aspect, provided herein is an antibody that binds TRDV2, comprising: (i) a VH comprising a VH CDR1, a VH CDR2, and a VH CDR3 having an amino acid sequence of SEQ ID NOs:3849, 3850, and 3851, respectively, and (ii) a VL comprising a VL CDR1, VL CDR2, and VL CDR3 having an amino acid sequence of SEQ ID NOs:3852, 3853, and 3854, respectively. In one aspect, provided herein is an antibody that binds TRDV2, comprising: (i) a VH comprising a VH CDR1, a VH CDR2, and a VH CDR3 having an amino acid sequence of SEQ ID NOs:3855, 3856, and 3857, respectively, and (ii) a VL comprising a VL CDR1, VL CDR2, and VL CDR3 having an amino acid sequence of SEQ ID NOs:3858, 3859, and 3860, respectively. In one aspect, provided herein is an antibody that binds TRDV2, comprising: (i) a VH comprising a VH CDR1, a VH CDR2, and a VH CDR3 having an amino acid sequence of SEQ ID NOs:3861, 3862, and 3863, respectively, and (ii) a VL comprising a VL CDR1, VL CDR2, and VL CDR3 having an amino acid sequence of SEQ ID NOs:3864, 3865, and 3866, respectively. In one aspect, provided herein is an antibody that binds TRDV2, comprising: (i) a VH comprising a VH CDR1, a VH CDR2, and a VH CDR3 having an amino acid sequence of SEQ ID NOs:3867, 3868, and 3869, respectively, and (ii) a VL comprising a VL CDR1, VL CDR2, and VL CDR3 having an amino acid sequence of SEQ ID NOs:3870, 3871, and 3872, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3873; and (ii) a VL comprising a VL CDR1, a VL CDR2, and a VL CDR3 having an amino acid sequence of a VL CDR1, a VL CDR2, and a VL CDR3, respectively, of SEQ ID NO:3874. In one aspect, provided herein is an antibody that binds TRDV2, comprising a VH having an amino acid sequence of SEQ ID NO:3873. In one aspect, provided herein is an antibody that binds TRDV2, comprising a VL having an amino acid sequence of SEQ ID NO:3874. In one aspect, provided herein is an antibody that binds TRDV2, comprising a VH having an amino acid sequence of SEQ ID NO:3873, and a VL having an amino acid sequence of SEQ ID NO:3874. In one aspect, provided herein is an antibody that binds TRDV2, comprising a heavy chain having an amino acid sequence of SEQ ID NO:3875. In one aspect, provided herein is an antibody that binds TRDV2, comprising a light chain having an amino acid sequence of SEQ ID NO:3876. In one aspect, provided herein is an antibody that binds TRDV2, comprising a heavy chain having an amino acid sequence of SEQ ID NO:3875, and a light chain having an amino acid sequence of SEQ ID NO:3876. In one aspect, provided herein is an antibody that binds TRDV2, comprising a VH having an amino acid sequence having at least 95% identity to an amino acid sequence of SEQ ID NO:3873. In one aspect, provided herein is an antibody that binds TRDV2, comprising a VL having an amino acid sequence having at least 95% identity to an amino acid sequence of SEQ ID NO:3874. In one aspect, provided herein is an antibody that binds TRDV2, comprising a VH having an amino acid sequence having at least 95% identity to an amino acid sequence of SEQ ID NO:3873, and a VL having an amino acid sequence having at least 95% identity to an amino acid sequence of SEQ ID NO:3874. In one aspect, provided herein is an antibody that binds TRDV2, comprising a heavy chain having an amino acid sequence having at least 95% identity to an amino acid sequence of SEQ ID NO:3875. In one aspect, provided herein is an antibody that binds TRDV2, comprising a light chain having an amino acid sequence having at least 95% identity to an amino acid sequence of SEQ ID NO:3876. In one aspect, provided herein is an antibody that binds TRDV2, comprising a heavy chain having an amino acid sequence having at least 95% identity to an amino acid sequence of SEQ ID NO:3875, and a light chain having an amino acid sequence having at least 95% identity to an amino acid sequence of SEQ ID NO:3876.

In one aspect, provided herein is an antibody that binds TRDV2, comprising: (i) a VH comprising a VH CDR1, a VH CDR2, and a VH CDR3 having an amino acid sequence of SEQ ID NOs:3877, 3878, and 3879, respectively, and (ii) a VL comprising a VL CDR1, VL CDR2, and VL CDR3 having an amino acid sequence of SEQ ID NOs:3880, 3881, and 3882, respectively. In one aspect, provided herein is an antibody that binds TRDV2, comprising: (i) a VH comprising a VH CDR1, a VH CDR2, and a VH CDR3 having an amino acid sequence of SEQ ID NOs:3883, 3884, and 3885, respectively, and (ii) a VL comprising a VL CDR1, VL CDR2, and VL CDR3 having an amino acid sequence of SEQ ID NOs:3886, 3887, and 3888, respectively. In one aspect, provided herein is an antibody that binds TRDV2, comprising: (i) a VH comprising a VH CDR1, a VH CDR2, and a VH CDR3 having an amino acid sequence of SEQ ID NOs:3889, 3890, and 3891, respectively, and (ii) a VL comprising a VL CDR1, VL CDR2, and VL CDR3 having an amino acid sequence of SEQ ID NOs:3892, 3893, and 3894, respectively. In one aspect, provided herein is an antibody that binds TRDV2, comprising: (i) a VH comprising a VH CDR1, a VH CDR2, and a VH CDR3 having an amino acid sequence of SEQ ID NOs:3895, 3896, and 3897, respectively, and (ii) a VL comprising a VL CDR1, VL CDR2, and VL CDR3 having an amino acid sequence of SEQ ID NOs:3898, 3899, and 3900, respectively. In one aspect, provided herein is an antibody that binds TRDV2, comprising: (i) a VH comprising a VH CDR1, a VH CDR2, and a VH CDR3 having an amino acid sequence of SEQ ID NOs:3901, 3902, and 3903, respectively, and (ii) a VL comprising a VL CDR1, VL CDR2, and VL CDR3 having an amino acid sequence of SEQ ID NOs:3904, 3905, and 3906, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3907; and (ii) a VL comprising a VL CDR1, a VL CDR2, and a VL CDR3 having an amino acid sequence of a VL CDR1, a VL CDR2, and a VL CDR3, respectively, of SEQ ID NO:3908. In one aspect, provided herein is an antibody that binds TRDV2, comprising a VH having an amino acid sequence of SEQ ID NO:3907. In one aspect, provided herein is an antibody that binds TRDV2, comprising a VL having an amino acid sequence of SEQ ID NO:3908. In one aspect, provided herein is an antibody that binds TRDV2, comprising a VH having an amino acid sequence of SEQ ID NO:3907, and a VL having an amino acid sequence of SEQ ID NO:3908. In one aspect, provided herein is an antibody that binds TRDV2, comprising a heavy chain having an amino acid sequence of SEQ ID NO:3909. In one aspect, provided herein is an antibody that binds TRDV2, comprising a light chain having an amino acid sequence of SEQ ID NO:3910. In one aspect, provided herein is an antibody that binds TRDV2, comprising a heavy chain having an amino acid sequence of SEQ ID NO:3909, and a light chain having an amino acid sequence of SEQ ID NO:3910. In one aspect, provided herein is an antibody that binds TRDV2, comprising a VH having an amino acid sequence having at least 95% identity to an amino acid sequence of SEQ ID NO:3907. In one aspect, provided herein is an antibody that binds TRDV2, comprising a VL having an amino acid sequence having at least 95% identity to an amino acid sequence of SEQ ID NO:3908. In one aspect, provided herein is an antibody that binds TRDV2, comprising a VH having an amino acid sequence having at least 95% identity to an amino acid sequence of SEQ ID NO:3907, and a VL having an amino acid sequence having at least 95% identity to an amino acid sequence of SEQ ID NO:3908. In one aspect, provided herein is an antibody that binds TRDV2, comprising a heavy chain having an amino acid sequence having at least 95% identity to an amino acid sequence of SEQ ID NO:3909. In one aspect, provided herein is an antibody that binds TRDV2, comprising a light chain having an amino acid sequence having at least 95% identity to an amino acid sequence of SEQ ID NO:3910. In one aspect, provided herein is an antibody that binds TRDV2, comprising a heavy chain having an amino acid sequence having at least 95% identity to an amino acid sequence of SEQ ID NO:3909, and a light chain having an amino acid sequence having at least 95% identity to an amino acid sequence of SEQ ID NO:3910.

In one aspect, provided herein is an antibody that binds TRDV2, comprising: (i) a VH comprising a VH CDR1, a VH CDR2, and a VH CDR3 having an amino acid sequence of SEQ ID NOs:3911, 3912, and 3913, respectively, and (ii) a VL comprising a VL CDR1, VL CDR2, and VL CDR3 having an amino acid sequence of SEQ ID NOs:3914, 3915, and 3916, respectively. In one aspect, provided herein is an antibody that binds TRDV2, comprising: (i) a VH comprising a VH CDR1, a VH CDR2, and a VH CDR3 having an amino acid sequence of SEQ ID NOs:3917, 3918, and 3919, respectively, and (ii) a VL comprising a VL CDR1, VL CDR2, and VL CDR3 having an amino acid sequence of SEQ ID NOs:3920, 3921, and 3922, respectively. In one aspect, provided herein is an antibody that binds TRDV2, comprising: (i) a VH comprising a VH CDR1, a VH CDR2, and a VH CDR3 having an amino acid sequence of SEQ ID NOs:3923, 3924, and 3925, respectively, and (ii) a VL comprising a VL CDR1, VL CDR2, and VL CDR3 having an amino acid sequence of SEQ ID NOs:3926, 3927, and 3928, respectively. In one aspect, provided herein is an antibody that binds TRDV2, comprising: (i) a VH comprising a VH CDR1, a VH CDR2, and a VH CDR3 having an amino acid sequence of SEQ ID NOs:3929, 3930, and 3931, respectively, and (ii) a VL comprising a VL CDR1, VL CDR2, and VL CDR3 having an amino acid sequence of SEQ ID NOs:3932, 3933, and 3934, respectively. In one aspect, provided herein is an antibody that binds TRDV2, comprising: (i) a VH comprising a VH CDR1, a VH CDR2, and a VH CDR3 having an amino acid sequence of SEQ ID NOs:3935, 3936, and 3937, respectively, and (ii) a VL comprising a VL CDR1, VL CDR2, and VL CDR3 having an amino acid sequence of SEQ ID NOs:3938, 3939, and 3940, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3941; and (ii) a VL comprising a VL CDR1, a VL CDR2, and a VL CDR3 having an amino acid sequence of a VL CDR1, a VL CDR2, and a VL CDR3, respectively, of SEQ ID NO:3942. In one aspect, provided herein is an antibody that binds TRDV2, comprising a VH having an amino acid sequence of SEQ ID NO:3941. In one aspect, provided herein is an antibody that binds TRDV2, comprising a VL having an amino acid sequence of SEQ ID NO:3942. In one aspect, provided herein is an antibody that binds TRDV2, comprising a VH having an amino acid sequence of SEQ ID NO:3941, and a VL having an amino acid sequence of SEQ ID NO:3942. In one aspect, provided herein is an antibody that binds TRDV2, comprising a heavy chain having an amino acid sequence of SEQ ID NO:3943. In one aspect, provided herein is an antibody that binds TRDV2, comprising a light chain having an amino acid sequence of SEQ ID NO:3944. In one aspect, provided herein is an antibody that binds TRDV2, comprising a heavy chain having an amino acid sequence of SEQ ID NO:3943, and a light chain having an amino acid sequence of SEQ ID NO:3944. In one aspect, provided herein is an antibody that binds TRDV2, comprising a VH having an amino acid sequence having at least 95% identity to an amino acid sequence of SEQ ID NO:3941. In one aspect, provided herein is an antibody that binds TRDV2, comprising a VL having an amino acid sequence having at least 95% identity to an amino acid sequence of SEQ ID NO:3942. In one aspect, provided herein is an antibody that binds TRDV2, comprising a VH having an amino acid sequence having at least 95% identity to an amino acid sequence of SEQ ID NO:3941, and a VL having an amino acid sequence having at least 95% identity to an amino acid sequence of SEQ ID NO:3942. In one aspect, provided herein is an antibody that binds TRDV2, comprising a heavy chain having an amino acid sequence having at least 95% identity to an amino acid sequence of SEQ ID NO:3943. In one aspect, provided herein is an antibody that binds TRDV2, comprising a light chain having an amino acid sequence having at least 95% identity to an amino acid sequence of SEQ ID NO:3944. In one aspect, provided herein is an antibody that binds TRDV2, comprising a heavy chain having an amino acid sequence having at least 95% identity to an amino acid sequence of SEQ ID NO:3943, and a light chain having an amino acid sequence having at least 95% identity to an amino acid sequence of SEQ ID NO:3944.

In one aspect, provided herein is an antibody that binds TRDV2, comprising: (i) a VH comprising a VH CDR1, a VH CDR2, and a VH CDR3 having an amino acid sequence of SEQ ID NOs:3945, 3946, and 3947, respectively, and (ii) a VL comprising a VL CDR1, VL CDR2, and VL CDR3 having an amino acid sequence of SEQ ID NOs:3948, 3949, and 3950, respectively. In one aspect, provided herein is an antibody that binds TRDV2, comprising: (i) a VH comprising a VH CDR1, a VH CDR2, and a VH CDR3 having an amino acid sequence of SEQ ID NOs:3951, 3952, and 3953, respectively, and (ii) a VL comprising a VL CDR1, VL CDR2, and VL CDR3 having an amino acid sequence of SEQ ID NOs:3954, 3955, and 3956, respectively. In one aspect, provided herein is an antibody that binds TRDV2, comprising: (i) a VH comprising a VH CDR1, a VH CDR2, and a VH CDR3 having an amino acid sequence of SEQ ID NOs:3957, 3958, and 3959, respectively, and (ii) a VL comprising a VL CDR1, VL CDR2, and VL CDR3 having an amino acid sequence of SEQ ID NOs:3960, 3961, and 3962, respectively. In one aspect, provided herein is an antibody that binds TRDV2, comprising: (i) a VH comprising a VH CDR1, a VH CDR2, and a VH CDR3 having an amino acid sequence of SEQ ID NOs:3963, 3964, and 3965, respectively, and (ii) a VL comprising a VL CDR1, VL CDR2, and VL CDR3 having an amino acid sequence of SEQ ID NOs:3966, 3967, and 3968, respectively. In one aspect, provided herein is an antibody that binds TRDV2, comprising: (i) a VH comprising a VH CDR1, a VH CDR2, and a VH CDR3 having an amino acid sequence of SEQ ID NOs:3969, 3970, and 3971, respectively, and (ii) a VL comprising a VL CDR1, VL CDR2, and VL CDR3 having an amino acid sequence of SEQ ID NOs:3972, 3973, and 3974, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3975; and (ii) a VL comprising a VL CDR1, a VL CDR2, and a VL CDR3 having an amino acid sequence of a VL CDR1, a VL CDR2, and a VL CDR3, respectively, of SEQ ID NO:3976. In one aspect, provided herein is an antibody that binds TRDV2, comprising a VH having an amino acid sequence of SEQ ID NO:3975. In one aspect, provided herein is an antibody that binds TRDV2, comprising a VL having an amino acid sequence of SEQ ID NO:3976. In one aspect, provided herein is an antibody that binds TRDV2, comprising a VH having an amino acid sequence of SEQ ID NO:3975, and a VL having an amino acid sequence of SEQ ID NO:3976. In one aspect, provided herein is an antibody that binds TRDV2, comprising a heavy chain having an amino acid sequence of SEQ ID NO:3977. In one aspect, provided herein is an antibody that binds TRDV2, comprising a light chain having an amino acid sequence of SEQ ID NO:3978. In one aspect, provided herein is an antibody that binds TRDV2, comprising a heavy chain having an amino acid sequence of SEQ ID NO:3977, and a light chain having an amino acid sequence of SEQ ID NO:3978. In one aspect, provided herein is an antibody that binds TRDV2, comprising a VH having an amino acid sequence having at least 95% identity to an amino acid sequence of SEQ ID NO:3975. In one aspect, provided herein is an antibody that binds TRDV2, comprising a VL having an amino acid sequence having at least 95% identity to an amino acid sequence of SEQ ID NO:3976. In one aspect, provided herein is an antibody that binds TRDV2, comprising a VH having an amino acid sequence having at least 95% identity to an amino acid sequence of SEQ ID NO:3975, and a VL having an amino acid sequence having at least 95% identity to an amino acid sequence of SEQ ID NO:3976. In one aspect, provided herein is an antibody that binds TRDV2, comprising a heavy chain having an amino acid sequence having at least 95% identity to an amino acid sequence of SEQ ID NO:3977. In one aspect, provided herein is an antibody that binds TRDV2, comprising a light chain having an amino acid sequence having at least 95% identity to an amino acid sequence of SEQ ID NO:3978. In one aspect, provided herein is an antibody that binds TRDV2, comprising a heavy chain having an amino acid sequence having at least 95% identity to an amino acid sequence of SEQ ID NO:3977, and a light chain having an amino acid sequence having at least 95% identity to an amino acid sequence of SEQ ID NO:3978.

In one aspect, provided herein is an antibody that binds TRDV2, comprising: (i) a VH comprising a VH CDR1, a VH CDR2, and a VH CDR3 having an amino acid sequence of SEQ ID NOs:3979, 3980, and 3981, respectively, and (ii) a VL comprising a VL CDR1, VL CDR2, and VL CDR3 having an amino acid sequence of SEQ ID NOs:3982, 3983, and 3984, respectively. In one aspect, provided herein is an antibody that binds TRDV2, comprising: (i) a VH comprising a VH CDR1, a VH CDR2, and a VH CDR3 having an amino acid sequence of SEQ ID NOs:3985, 3986, and 3987, respectively, and (ii) a VL comprising a VL CDR1, VL CDR2, and VL CDR3 having an amino acid sequence of SEQ ID NOs:3988, 3989, and 3990, respectively. In one aspect, provided herein is an antibody that binds TRDV2, comprising: (i) a VH comprising a VH CDR1, a VH CDR2, and a VH CDR3 having an amino acid sequence of SEQ ID NOs:3991, 3992, and 3993, respectively, and (ii) a VL comprising a VL CDR1, VL CDR2, and VL CDR3 having an amino acid sequence of SEQ ID NOs:3994, 3995, and 3996, respectively. In one aspect, provided herein is an antibody that binds TRDV2, comprising: (i) a VH comprising a VH CDR1, a VH CDR2, and a VH CDR3 having an amino acid sequence of SEQ ID NOs:3997, 3998, and 3999, respectively, and (ii) a VL comprising a VL CDR1, VL CDR2, and VL CDR3 having an amino acid sequence of SEQ ID NOs:4000, 4001, and 4002, respectively. In one aspect, provided herein is an antibody that binds TRDV2, comprising: (i) a VH comprising a VH CDR1, a VH CDR2, and a VH CDR3 having an amino acid sequence of SEQ ID NOs:4003, 4004, and 4005, respectively, and (ii) a VL comprising a VL CDR1, VL CDR2, and VL CDR3 having an amino acid sequence of SEQ ID NOs:4006, 4007, and 4008, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4009; and (ii) a VL comprising a VL CDR1, a VL CDR2, and a VL CDR3 having an amino acid sequence of a VL CDR1, a VL CDR2, and a VL CDR3, respectively, of SEQ ID NO:4010. In one aspect, provided herein is an antibody that binds TRDV2, comprising a VH having an amino acid sequence of SEQ ID NO:4009. In one aspect, provided herein is an antibody that binds TRDV2, comprising a VL having an amino acid sequence of SEQ ID NO:4010. In one aspect, provided herein is an antibody that binds TRDV2, comprising a VH having an amino acid sequence of SEQ ID NO:4009, and a VL having an amino acid sequence of SEQ ID NO:4010. In one aspect, provided herein is an antibody that binds TRDV2, comprising a heavy chain having an amino acid sequence of SEQ ID NO:4011. In one aspect, provided herein is an antibody that binds TRDV2, comprising a light chain having an amino acid sequence of SEQ ID NO:4012. In one aspect, provided herein is an antibody that binds TRDV2, comprising a heavy chain having an amino acid sequence of SEQ ID NO:4011, and a light chain having an amino acid sequence of SEQ ID NO:4012. In one aspect, provided herein is an antibody that binds TRDV2, comprising a VH having an amino acid sequence having at least 95% identity to an amino acid sequence of SEQ ID NO:4009. In one aspect, provided herein is an antibody that binds TRDV2, comprising a VL having an amino acid sequence having at least 95% identity to an amino acid sequence of SEQ ID NO:4010. In one aspect, provided herein is an antibody that binds TRDV2, comprising a VH having an amino acid sequence having at least 95% identity to an amino acid sequence of SEQ ID NO:4009, and a VL having an amino acid sequence having at least 95% identity to an amino acid sequence of SEQ ID NO:4010. In one aspect, provided herein is an antibody that binds TRDV2, comprising a heavy chain having an amino acid sequence having at least 95% identity to an amino acid sequence of SEQ ID NO:4011. In one aspect, provided herein is an antibody that binds TRDV2, comprising a light chain having an amino acid sequence having at least 95% identity to an amino acid sequence of SEQ ID NO:4012. In one aspect, provided herein is an antibody that binds TRDV2, comprising a heavy chain having an amino acid sequence having at least 95% identity to an amino acid sequence of SEQ ID NO:4011, and a light chain having an amino acid sequence having at least 95% identity to an amino acid sequence of SEQ ID NO:4012.

In one aspect, provided herein is an antibody that binds TRDV2, comprising: (i) a VH comprising a VH CDR1, a VH CDR2, and a VH CDR3 having an amino acid sequence of SEQ ID NOs:4013, 4014, and 4015, respectively, and (ii) a VL comprising a VL CDR1, VL CDR2, and VL CDR3 having an amino acid sequence of SEQ ID NOs:4016, 4017, and 4018, respectively. In one aspect, provided herein is an antibody that binds TRDV2, comprising: (i) a VH comprising a VH CDR1, a VH CDR2, and a VH CDR3 having an amino acid sequence of SEQ ID NOs:4019, 4020, and 4021, respectively, and (ii) a VL comprising a VL CDR1, VL CDR2, and VL CDR3 having an amino acid sequence of SEQ ID NOs:4022, 4023, and 4024, respectively. In one aspect, provided herein is an antibody that binds TRDV2, comprising: (i) a VH comprising a VH CDR1, a VH CDR2, and a VH CDR3 having an amino acid sequence of SEQ ID NOs:4025, 4026, and 4027, respectively, and (ii) a VL comprising a VL CDR1, VL CDR2, and VL CDR3 having an amino acid sequence of SEQ ID NOs:4028, 4029, and 4030, respectively. In one aspect, provided herein is an antibody that binds TRDV2, comprising: (i) a VH comprising a VH CDR1, a VH CDR2, and a VH CDR3 having an amino acid sequence of SEQ ID NOs:4031, 4032, and 4033, respectively, and (ii) a VL comprising a VL CDR1, VL CDR2, and VL CDR3 having an amino acid sequence of SEQ ID NOs:4034, 4035, and 4036, respectively. In one aspect, provided herein is an antibody that binds TRDV2, comprising: (i) a VH comprising a VH CDR1, a VH CDR2, and a VH CDR3 having an amino acid sequence of SEQ ID NOs:4037, 4038, and 4039, respectively, and (ii) a VL comprising a VL CDR1, VL CDR2, and VL CDR3 having an amino acid sequence of SEQ ID NOs:4040, 4041, and 4042, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4043; and (ii) a VL comprising a VL CDR1, a VL CDR2, and a VL CDR3 having an amino acid sequence of a VL CDR1, a VL CDR2, and a VL CDR3, respectively, of SEQ ID NO:4044. In one aspect, provided herein is an antibody that binds TRDV2, comprising a VH having an amino acid sequence of SEQ ID NO:4043. In one aspect, provided herein is an antibody that binds TRDV2, comprising a VL having an amino acid sequence of SEQ ID NO:4044. In one aspect, provided herein is an antibody that binds TRDV2, comprising a VH having an amino acid sequence of SEQ ID NO:4043, and a VL having an amino acid sequence of SEQ ID NO:4044. In one aspect, provided herein is an antibody that binds TRDV2, comprising a heavy chain having an amino acid sequence of SEQ ID NO:4045. In one aspect, provided herein is an antibody that binds TRDV2, comprising a light chain having an amino acid sequence of SEQ ID NO:4046. In one aspect, provided herein is an antibody that binds TRDV2, comprising a heavy chain having an amino acid sequence of SEQ ID NO:4045, and a light chain having an amino acid sequence of SEQ ID NO:4046. In one aspect, provided herein is an antibody that binds TRDV2, comprising a VH having an amino acid sequence having at least 95% identity to an amino acid sequence of SEQ ID NO:4043. In one aspect, provided herein is an antibody that binds TRDV2, comprising a VL having an amino acid sequence having at least 95% identity to an amino acid sequence of SEQ ID NO:4044. In one aspect, provided herein is an antibody that binds TRDV2, comprising a VH having an amino acid sequence having at least 95% identity to an amino acid sequence of SEQ ID NO:4043, and a VL having an amino acid sequence having at least 95% identity to an amino acid sequence of SEQ ID NO:4044. In one aspect, provided herein is an antibody that binds TRDV2, comprising a heavy chain having an amino acid sequence having at least 95% identity to an amino acid sequence of SEQ ID NO:4045. In one aspect, provided herein is an antibody that binds TRDV2, comprising a light chain having an amino acid sequence having at least 95% identity to an amino acid sequence of SEQ ID NO:4046. In one aspect, provided herein is an antibody that binds TRDV2, comprising a heavy chain having an amino acid sequence having at least 95% identity to an amino acid sequence of SEQ ID NO:4045, and a light chain having an amino acid sequence having at least 95% identity to an amino acid sequence of SEQ ID NO:4046.

In one aspect, provided herein is an antibody that binds TRDV2, comprising: (i) a VH comprising a VH CDR1, a VH CDR2, and a VH CDR3 having an amino acid sequence of SEQ ID NOs:4047, 4048, and 4049, respectively, and (ii) a VL comprising a VL CDR1, VL CDR2, and VL CDR3 having an amino acid sequence of SEQ ID NOs:4050, 4051, and 4052, respectively. In one aspect, provided herein is an antibody that binds TRDV2, comprising: (i) a VH comprising a VH CDR1, a VH CDR2, and a VH CDR3 having an amino acid sequence of SEQ ID NOs:4053, 4054, and 4055, respectively, and (ii) a VL comprising a VL CDR1, VL CDR2, and VL CDR3 having an amino acid sequence of SEQ ID NOs:4056, 4057, and 4058, respectively. In one aspect, provided herein is an antibody that binds TRDV2, comprising: (i) a VH comprising a VH CDR1, a VH CDR2, and a VH CDR3 having an amino acid sequence of SEQ ID NOs:4059, 4060, and 4061, respectively, and (ii) a VL comprising a VL CDR1, VL CDR2, and VL CDR3 having an amino acid sequence of SEQ ID NOs:4062, 4063, and 4064, respectively. In one aspect, provided herein is an antibody that binds TRDV2, comprising: (i) a VH comprising a VH CDR1, a VH CDR2, and a VH CDR3 having an amino acid sequence of SEQ ID NOs:4065, 4066, and 4067, respectively, and (ii) a VL comprising a VL CDR1, VL CDR2, and VL CDR3 having an amino acid sequence of SEQ ID NOs:4068, 4069, and 4070, respectively. In one aspect, provided herein is an antibody that binds TRDV2, comprising: (i) a VH comprising a VH CDR1, a VH CDR2, and a VH CDR3 having an amino acid sequence of SEQ ID NOs:4071, 4072, and 4073, respectively, and (ii) a VL comprising a VL CDR1, VL CDR2, and VL CDR3 having an amino acid sequence of SEQ ID NOs:4074, 4075, and 4076, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4077; and (ii) a VL comprising a VL CDR1, a VL CDR2, and a VL CDR3 having an amino acid sequence of a VL CDR1, a VL CDR2, and a VL CDR3, respectively, of SEQ ID NO:4078. In one aspect, provided herein is an antibody that binds TRDV2, comprising a VH having an amino acid sequence of SEQ ID NO:4077. In one aspect, provided herein is an antibody that binds TRDV2, comprising a VL having an amino acid sequence of SEQ ID NO:4078. In one aspect, provided herein is an antibody that binds TRDV2, comprising a VH having an amino acid sequence of SEQ ID NO:4077, and a VL having an amino acid sequence of SEQ ID NO:4078. In one aspect, provided herein is an antibody that binds TRDV2, comprising a heavy chain having an amino acid sequence of SEQ ID NO:4079. In one aspect, provided herein is an antibody that binds TRDV2, comprising a light chain having an amino acid sequence of SEQ ID NO:4080. In one aspect, provided herein is an antibody that binds TRDV2, comprising a heavy chain having an amino acid sequence of SEQ ID NO:4079, and a light chain having an amino acid sequence of SEQ ID NO:4080. In one aspect, provided herein is an antibody that binds TRDV2, comprising a VH having an amino acid sequence having at least 95% identity to an amino acid sequence of SEQ ID NO:4077. In one aspect, provided herein is an antibody that binds TRDV2, comprising a VL having an amino acid sequence having at least 95% identity to an amino acid sequence of SEQ ID NO:4078. In one aspect, provided herein is an antibody that binds TRDV2, comprising a VH having an amino acid sequence having at least 95% identity to an amino acid sequence of SEQ ID NO:4077, and a VL having an amino acid sequence having at least 95% identity to an amino acid sequence of SEQ ID NO:4078. In one aspect, provided herein is an antibody that binds TRDV2, comprising a heavy chain having an amino acid sequence having at least 95% identity to an amino acid sequence of SEQ ID NO:4079. In one aspect, provided herein is an antibody that binds TRDV2, comprising a light chain having an amino acid sequence having at least 95% identity to an amino acid sequence of SEQ ID NO:4080. In one aspect, provided herein is an antibody that binds TRDV2, comprising a heavy chain having an amino acid sequence having at least 95% identity to an amino acid sequence of SEQ ID NO:4079, and a light chain having an amino acid sequence having at least 95% identity to an amino acid sequence of SEQ ID NO:4080.

In one aspect, provided herein is an antibody that binds TRDV2, comprising: (i) a VH comprising a VH CDR1, a VH CDR2, and a VH CDR3 having an amino acid sequence of SEQ ID NOs:4081, 4082, and 4083, respectively, and (ii) a VL comprising a VL CDR1, VL CDR2, and VL CDR3 having an amino acid sequence of SEQ ID NOs:4084, 4085, and 4086, respectively. In one aspect, provided herein is an antibody that binds TRDV2, comprising: (i) a VH comprising a VH CDR1, a VH CDR2, and a VH CDR3 having an amino acid sequence of SEQ ID NOs:4087, 4088, and 4089, respectively, and (ii) a VL comprising a VL CDR1, VL CDR2, and VL CDR3 having an amino acid sequence of SEQ ID NOs:4090, 4091, and 4092, respectively. In one aspect, provided herein is an antibody that binds TRDV2, comprising: (i) a VH comprising a VH CDR1, a VH CDR2, and a VH CDR3 having an amino acid sequence of SEQ ID NOs:4093, 4094, and 4095, respectively, and (ii) a VL comprising a VL CDR1, VL CDR2, and VL CDR3 having an amino acid sequence of SEQ ID NOs:4096, 4097, and 4098, respectively. In one aspect, provided herein is an antibody that binds TRDV2, comprising: (i) a VH comprising a VH CDR1, a VH CDR2, and a VH CDR3 having an amino acid sequence of SEQ ID NOs:4099, 4100, and 4101, respectively, and (ii) a VL comprising a VL CDR1, VL CDR2, and VL CDR3 having an amino acid sequence of SEQ ID NOs:4102, 4103, and 4104, respectively. In one aspect, provided herein is an antibody that binds TRDV2, comprising: (i) a VH comprising a VH CDR1, a VH CDR2, and a VH CDR3 having an amino acid sequence of SEQ ID NOs:4105, 4106, and 4107, respectively, and (ii) a VL comprising a VL CDR1, VL CDR2, and VL CDR3 having an amino acid sequence of SEQ ID NOs:4108, 4109, and 4110, respectively. In one aspect, provided herein is an antibody that binds TRDV2, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4111; and (ii) a VL comprising a VL CDR1, a VL CDR2, and a VL CDR3 having an amino acid sequence of a VL CDR1, a VL CDR2, and a VL CDR3, respectively, of SEQ ID NO:4112. In one aspect, provided herein is an antibody that binds TRDV2, comprising a VH having an amino acid sequence of SEQ ID NO:4111. In one aspect, provided herein is an antibody that binds TRDV2, comprising a VL having an amino acid sequence of SEQ ID NO:4112. In one aspect, provided herein is an antibody that binds TRDV2, comprising a VH having an amino acid sequence of SEQ ID NO:4111, and a VL having an amino acid sequence of SEQ ID NO:4112. In one aspect, provided herein is an antibody that binds TRDV2, comprising a heavy chain having an amino acid sequence of SEQ ID NO:4113. In one aspect, provided herein is an antibody that binds TRDV2, comprising a light chain having an amino acid sequence of SEQ ID NO:4114. In one aspect, provided herein is an antibody that binds TRDV2, comprising a heavy chain having an amino acid sequence of SEQ ID NO:4113, and a light chain having an amino acid sequence of SEQ ID NO:4114. In one aspect, provided herein is an antibody that binds TRDV2, comprising a VH having an amino acid sequence having at least 95% identity to an amino acid sequence of SEQ ID NO:4111. In one aspect, provided herein is an antibody that binds TRDV2, comprising a VL having an amino acid sequence having at least 95% identity to an amino acid sequence of SEQ ID NO:4112. In one aspect, provided herein is an antibody that binds TRDV2, comprising a VH having an amino acid sequence having at least 95% identity to an amino acid sequence of SEQ ID NO:4111, and a VL having an amino acid sequence having at least 95% identity to an amino acid sequence of SEQ ID NO:4112. In one aspect, provided herein is an antibody that binds TRDV2, comprising a heavy chain having an amino acid sequence having at least 95% identity to an amino acid sequence of SEQ ID NO:4113. In one aspect, provided herein is an antibody that binds TRDV2, comprising a light chain having an amino acid sequence having at least 95% identity to an amino acid sequence of SEQ ID NO:4114. In one aspect, provided herein is an antibody that binds TRDV2, comprising a heavy chain having an amino acid sequence having at least 95% identity to an amino acid sequence of SEQ ID NO:4113, and a light chain having an amino acid sequence having at least 95% identity to an amino acid sequence of SEQ ID NO:4114.

In one aspect, provided herein is an antibody that binds TRDV2. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2037. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2038. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2037; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2038. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2071. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2072. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2071; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2072. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2105. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2106. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2105; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2106. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2139. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2140. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2139; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2140. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2173. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2174. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2173; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2174. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2207. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2208. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2207; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2208. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2241. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2242. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2241; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2242. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2275. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2276. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2275; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2276. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2309. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2310. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2309; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2310. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2343. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2344. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2343; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2344. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2377. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2378. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2377; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2378. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2411. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2412. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2411; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2412. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2445. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2446. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2445; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2446. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2479. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2480. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2479; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2480. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2513. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2514. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2513; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2514. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2547. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2548. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2547; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2548. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2581. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2582. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2581; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2582. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2615. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2616. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2615; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2616. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2649. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2650. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2649; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2650. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2683. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2684. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2683; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2684. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2717. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2718. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2717; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2718. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2751. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2752. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2751; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2752. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2785. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2786. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2785; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2786. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2819. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2820. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2819; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2820. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2853. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2854. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2853; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2854. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2887. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2888. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2887; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2888. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2921. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2922. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2921; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2922. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2955. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2956. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2955; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2956. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2989. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2990. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2989; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2990. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3023. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3024. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3023; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3024. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3057. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3058. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3057; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3058. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3091. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3092. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3091; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3092. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3125. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3126. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3125; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3126. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3159. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3160. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3159; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3160. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3193. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3194. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3193; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3194. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3227. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3228. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3227; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3228. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3261. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3262. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3261; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3262. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3295. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3296. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3295; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3296. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3329. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3330. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3329; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3330. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3363. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3364. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3363; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3364. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3397. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3398. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3397; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3398. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3431. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3432. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3431; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3432. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3465. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3466. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3465; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3466. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3499. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3500. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3499; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3500. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3533. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3534. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3533; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3534. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3567. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3568. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3567; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3568. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3601. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3602. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3601; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3602. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3635. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3636. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3635; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3636. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3669. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3670. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3669; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3670. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3703. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3704. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3703; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3704. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3737. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3738. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3737; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3738. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3771. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3772. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3771; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3772. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3805. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3806. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3805; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3806. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3839. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3840. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3839; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3840. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3873. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3874. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3873; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3874. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3907. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3908. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3907; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3908. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3941. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3942. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3941; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3942. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3975. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3976. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3975; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3976. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4009. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4010. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4009; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4010. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4043. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4044. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4043; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4044. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4077. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4078. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4077; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4078. In one embodiment, the TRDV2 antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4111. In one embodiment, the TRDV2 antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4112. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4111; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4112. In some embodiments, the TRDV2 antibody is a multispecific antibody. In some embodiments, the TRDV2 antibody is a bispecific antibody. In some embodiments, the TRDV2 antibody is a trispecific antibody. In some embodiments, the TRDV2 antibody is a quadraspecific antibody.

In one aspect, provided herein is an antibody that binds TRDV2, comprising a VH CDR1 having an amino acid sequence of SEQ ID NO:2031, wherein the fifth amino acid is R or S; a VH CDR2 having an amino acid sequence of SEQ ID NO:2032; a VH CDR3 having an amino acid sequence of SEQ ID NO:2033, wherein the sixteenth amino acid is F or Y. In one aspect, provided herein is an antibody that binds TRDV2, comprising a VL CDR1 having an amino acid sequence of SEQ ID NO:2034; a VL CDR2 having an amino acid sequence of SEQ ID NO:2035, wherein the third amino acid is S or T; and a VL CDR3 having an amino acid sequence of SEQ ID NO:2036, wherein the sixth amino acid is R or S, the seventh amino acid is L or M, the eighth amino acid is S or T. In one aspect, provided herein is an antibody that binds TRDV2, comprising a VH CDR1 having an amino acid sequence of SEQ ID NO:2031, wherein the fifth amino acid is R or S; a VH CDR2 having an amino acid sequence of SEQ ID NO:2032; a VH CDR3 having an amino acid sequence of SEQ ID NO:2033, wherein the sixteenth amino acid is F or Y; a VL CDR1 having an amino acid sequence of SEQ ID NO:2034; a VL CDR2 having an amino acid sequence of SEQ ID NO:2035, wherein the third amino acid is S or T; and a VL CDR3 having an amino acid sequence of SEQ ID NO:2036, wherein the sixth amino acid is R or S, the seventh amino acid is L or M, the eighth amino acid is S or T.

In one aspect, provided herein is an antibody that binds TRDV2, comprising a VH CDR1 having an amino acid sequence of SEQ ID NO:2099, wherein the fifth amino acid is S or T, the eighth amino acid is R or S; a VH CDR2 having an amino acid sequence of SEQ ID NO:2100, wherein the fourth amino acid is D or S, the sixth amino acid is I or S; a VH CDR3 having an amino acid sequence of SEQ ID NO:2101, wherein the third amino acid is D or E, the fourth amino acid is R or S, the sixth amino acid is Y or there is no amino acid added to the sixth position and sequence continues to the seventh position, the eighth amino acid is G or V, the ninth amino acid is P or S. In one aspect, provided herein is an antibody that binds TRDV2, comprising a VL CDR1 having an amino acid sequence of SEQ ID NO:2102; a VL CDR2 having an amino acid sequence of SEQ ID NO:2103; and a VL CDR3 having an amino acid sequence of SEQ ID NO:2104. In one aspect, provided herein is an antibody that binds TRDV2, comprising a VH CDR1 having an amino acid sequence of SEQ ID NO:2099, wherein the fifth amino acid is S or T, the eighth amino acid is R or S; a VH CDR2 having an amino acid sequence of SEQ ID NO:2100, wherein the fourth amino acid is D or S, the sixth amino acid is I or S; a VH CDR3 having an amino acid sequence of SEQ ID NO:2101, wherein the third amino acid is D or E, the fourth amino acid is R or S, the sixth amino acid is Y or there is no amino acid added to the sixth position and sequence continues to the seventh position, the eighth amino acid is G or V, the ninth amino acid is P or S; a VL CDR1 having an amino acid sequence of SEQ ID NO:2102; a VL CDR2 having an amino acid sequence of SEQ ID NO:2103; and a VL CDR3 having an amino acid sequence of SEQ ID NO:2104.

In one aspect, provided herein is an antibody that binds TRDV2, comprising a VH CDR1 having an amino acid sequence of SEQ ID NO:2133, wherein the second amino acid is D or G, the fifth amino acid is N, S, or T, the sixth amino acid is N or S, the seventh amino acid is H or Y; a VH CDR2 having an amino acid sequence of SEQ ID NO:2134, wherein the first amino acid is I or V, the fourth amino acid is N or S, the sixth amino acid is N or S; a VH CDR3 having an amino acid sequence of SEQ ID NO:2135, wherein the seventh amino acid is E or V, the tenth amino acid is G or T. In one aspect, provided herein is an antibody that binds TRDV2, comprising a VL CDR1 having an amino acid sequence of SEQ ID NO:2136; a VL CDR2 having an amino acid sequence of SEQ ID NO:2137; and a VL CDR3 having an amino acid sequence of SEQ ID NO:2138, wherein the ninth amino acid is G or S. In one aspect, provided herein is an antibody that binds TRDV2, comprising a VH CDR1 having an amino acid sequence of SEQ ID NO:2133, wherein the second amino acid is D or G, the fifth amino acid is N, S, or T, the sixth amino acid is N or S, the seventh amino acid is H or Y; a VH CDR2 having an amino acid sequence of SEQ ID NO:2134, wherein the first amino acid is I or V, the fourth amino acid is N or S, the sixth amino acid is N or S; a VH CDR3 having an amino acid sequence of SEQ ID NO:2135, wherein the seventh amino acid is E or V, the tenth amino acid is G or T; a VL CDR1 having an amino acid sequence of SEQ ID NO:2136; a VL CDR2 having an amino acid sequence of SEQ ID NO:2137; and a VL CDR3 having an amino acid sequence of SEQ ID NO:2138, wherein the ninth amino acid is G or S.

In one aspect, provided herein is an antibody that binds TRDV2, comprising a VH CDR1 having an amino acid sequence of SEQ ID NO:2269, wherein the first amino acid is G or S, the fifth amino acid is S or T; a VH CDR2 having an amino acid sequence of SEQ ID NO:2270; a VH CDR3 having an amino acid sequence of SEQ ID NO:2271, wherein the seventh amino acid is D or E, the ninth amino acid is D or S, the eighteenth amino acid is G or N. In one aspect, provided herein is an antibody that binds TRDV2, comprising a VL CDR1 having an amino acid sequence of SEQ ID NO:2272; a VL CDR2 having an amino acid sequence of SEQ ID NO:2273; and a VL CDR3 having an amino acid sequence of SEQ ID NO:2274, wherein the third amino acid is F or Y, the sixth amino acid is A or T. In one aspect, provided herein is an antibody that binds TRDV2, comprising a VH CDR1 having an amino acid sequence of SEQ ID NO:2269, wherein the first amino acid is G or S, the fifth amino acid is S or T; a VH CDR2 having an amino acid sequence of SEQ ID NO:2270; a VH CDR3 having an amino acid sequence of SEQ ID NO:2271, wherein the seventh amino acid is D or E, the ninth amino acid is D or S, the eighteenth amino acid is G or N; a VL CDR1 having an amino acid sequence of SEQ ID NO:2272; a VL CDR2 having an amino acid sequence of SEQ ID NO:2273; and a VL CDR3 having an amino acid sequence of SEQ ID NO:2274, wherein the third amino acid is F or Y, the sixth amino acid is A or T.

In one aspect, provided herein is an antibody that binds TRDV2, comprising a VH CDR1 having an amino acid sequence of SEQ ID NO:2337, wherein the first amino acid is G or N, the fifth amino acid is N or S, the sixth amino acid is G, N, or Y; a VH CDR2 having an amino acid sequence of SEQ ID NO:2338, wherein the second amino acid is F or Y; a VH CDR3 having an amino acid sequence of SEQ ID NO:2339, wherein the second amino acid is K or R, the third amino acid is E or L, the tenth amino acid is S or T, the eleventh amino acid is A or T. In one aspect, provided herein is an antibody that binds TRDV2, comprising a VL CDR1 having an amino acid sequence of SEQ ID NO:2340, wherein the first amino acid is S or T, the fifth amino acid is E or G, the seventh amino acid is D or N; a VL CDR2 having an amino acid sequence of SEQ ID NO:2341; and a VL CDR3 having an amino acid sequence of SEQ ID NO:2342, wherein the first amino acid is A or T. In one aspect, provided herein is an antibody that binds TRDV2, comprising a VH CDR1 having an amino acid sequence of SEQ ID NO:2337, wherein the first amino acid is G or N, the fifth amino acid is N or S, the sixth amino acid is G, N, or Y; a VH CDR2 having an amino acid sequence of SEQ ID NO:2338, wherein the second amino acid is F or Y; a VH CDR3 having an amino acid sequence of SEQ ID NO:2339, wherein the second amino acid is K or R, the third amino acid is E or L, the tenth amino acid is S or T, the eleventh amino acid is A or T; a VL CDR1 having an amino acid sequence of SEQ ID NO:2340, wherein the first amino acid is S or T, the fifth amino acid is E or G, the seventh amino acid is D or N; a VL CDR2 having an amino acid sequence of SEQ ID NO:2341; and a VL CDR3 having an amino acid sequence of SEQ ID NO:2342, wherein the first amino acid is A or T.

In one aspect, provided herein is an antibody that binds TRDV2, comprising a VH CDR1 having an amino acid sequence of SEQ ID NO:3017, wherein the sixth amino acid is S or T; a VH CDR2 having an amino acid sequence of SEQ ID NO:3018; a VH CDR3 having an amino acid sequence of SEQ ID NO:3019, wherein the eighth amino acid is S or T. In one aspect, provided herein is an antibody that binds TRDV2, comprising a VL CDR1 having an amino acid sequence of SEQ ID NO:3020, wherein the second amino acid is G or S; a VL CDR2 having an amino acid sequence of SEQ ID NO:3021; and a VL CDR3 having an amino acid sequence of SEQ ID NO:3022, wherein the first amino acid is I or M. In one aspect, provided herein is an antibody that binds TRDV2, comprising a VH CDR1 having an amino acid sequence of SEQ ID NO:3017, wherein the sixth amino acid is S or T; a VH CDR2 having an amino acid sequence of SEQ ID NO:3018; a VH CDR3 having an amino acid sequence of SEQ ID NO:3019, wherein the eighth amino acid is S or T; a VL CDR1 having an amino acid sequence of SEQ ID NO:3020, wherein the second amino acid is G or S; a VL CDR2 having an amino acid sequence of SEQ ID NO:3021; and a VL CDR3 having an amino acid sequence of SEQ ID NO:3022, wherein the first amino acid is I or M.

In one aspect, provided herein is an antibody that binds TRDV2, comprising a VH CDR1 having an amino acid sequence of SEQ ID NO:2983; a VH CDR2 having an amino acid sequence of SEQ ID NO:2984, wherein the third amino acid is P or S; a VH CDR3 having an amino acid sequence of SEQ ID NO:2985, wherein the fourth amino acid is M or V, the sixth amino acid is T or V, the eleventh amino acid is A or D. In one aspect, provided herein is an antibody that binds TRDV2, comprising a VL CDR1 having an amino acid sequence of SEQ ID NO:2986; a VL CDR2 having an amino acid sequence of SEQ ID NO:2987; and a VL CDR3 having an amino acid sequence of SEQ ID NO:2988. In one aspect, provided herein is an antibody that binds TRDV2, comprising a VH CDR1 having an amino acid sequence of SEQ ID NO:2983; a VH CDR2 having an amino acid sequence of SEQ ID NO:2984, wherein the third amino acid is P or S; a VH CDR3 having an amino acid sequence of SEQ ID NO:2985, wherein the fourth amino acid is M or V, the sixth amino acid is T or V, the eleventh amino acid is A or D; a VL CDR1 having an amino acid sequence of SEQ ID NO:2986; a VL CDR2 having an amino acid sequence of SEQ ID NO:2987; and a VL CDR3 having an amino acid sequence of SEQ ID NO:2988.

In one aspect, provided herein is an antibody that binds to TRGDC. In some embodiments, the antibody comprises a heavy chain variable region and a light chain variable region. In some embodiments, the TRGDC antibody is a humanized antibody.

In one aspect, provided herein is an antibody that binds TRGDC, comprising: (i) a VH comprising a VH CDR1, a VH CDR2, and a VH CDR3 having an amino acid sequence of SEQ ID NOs:4115, 4116, and 4117, respectively, and (ii) a VL comprising a VL CDR1, VL CDR2, and VL CDR3 having an amino acid sequence of SEQ ID NOs:4118, 4119, and 4120, respectively. In one aspect, provided herein is an antibody that binds TRGDC, comprising: (i) a VH comprising a VH CDR1, a VH CDR2, and a VH CDR3 having an amino acid sequence of SEQ ID NOs:4121, 4122, and 4123, respectively, and (ii) a VL comprising a VL CDR1, VL CDR2, and VL CDR3 having an amino acid sequence of SEQ ID NOs:4124, 4125, and 4126, respectively. In one aspect, provided herein is an antibody that binds TRGDC, comprising: (i) a VH comprising a VH CDR1, a VH CDR2, and a VH CDR3 having an amino acid sequence of SEQ ID NOs:4127, 4128, and 4129, respectively, and (ii) a VL comprising a VL CDR1, VL CDR2, and VL CDR3 having an amino acid sequence of SEQ ID NOs:4130, 4131, and 4132, respectively. In one aspect, provided herein is an antibody that binds TRGDC, comprising: (i) a VH comprising a VH CDR1, a VH CDR2, and a VH CDR3 having an amino acid sequence of SEQ ID NOs:4133, 4134, and 4135, respectively, and (ii) a VL comprising a VL CDR1, VL CDR2, and VL CDR3 having an amino acid sequence of SEQ ID NOs:4136, 4137, and 4138, respectively. In one aspect, provided herein is an antibody that binds TRGDC, comprising: (i) a VH comprising a VH CDR1, a VH CDR2, and a VH CDR3 having an amino acid sequence of SEQ ID NOs:4139, 4140, and 4141, respectively, and (ii) a VL comprising a VL CDR1, VL CDR2, and VL CDR3 having an amino acid sequence of SEQ ID NOs:4142, 4143, and 4144, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4145; and (ii) a VL comprising a VL CDR1, a VL CDR2, and a VL CDR3 having an amino acid sequence of a VL CDR1, a VL CDR2, and a VL CDR3, respectively, of SEQ ID NO:4146. In one aspect, provided herein is an antibody that binds TRGDC, comprising a VH having an amino acid sequence of SEQ ID NO:4145. In one aspect, provided herein is an antibody that binds TRGDC, comprising a VL having an amino acid sequence of SEQ ID NO:4146. In one aspect, provided herein is an antibody that binds TRGDC, comprising a VH having an amino acid sequence of SEQ ID NO:4145, and a VL having an amino acid sequence of SEQ ID NO:4146. In one aspect, provided herein is an antibody that binds TRGDC, comprising a heavy chain having an amino acid sequence of SEQ ID NO:4147. In one aspect, provided herein is an antibody that binds TRGDC, comprising a light chain having an amino acid sequence of SEQ ID NO:4148. In one aspect, provided herein is an antibody that binds TRGDC, comprising a heavy chain having an amino acid sequence of SEQ ID NO:4147, and a light chain having an amino acid sequence of SEQ ID NO:4148. In one aspect, provided herein is an antibody that binds TRGDC, comprising a VH having an amino acid sequence having at least 95% identity to an amino acid sequence of SEQ ID NO:4145. In one aspect, provided herein is an antibody that binds TRGDC, comprising a VL having an amino acid sequence having at least 95% identity to an amino acid sequence of SEQ ID NO:4146. In one aspect, provided herein is an antibody that binds TRGDC, comprising a VH having an amino acid sequence having at least 95% identity to an amino acid sequence of SEQ ID NO:4145, and a VL having an amino acid sequence having at least 95% identity to an amino acid sequence of SEQ ID NO:4146. In one aspect, provided herein is an antibody that binds TRGDC, comprising a heavy chain having an amino acid sequence having at least 95% identity to an amino acid sequence of SEQ ID NO:4147. In one aspect, provided herein is an antibody that binds TRGDC, comprising a light chain having an amino acid sequence having at least 95% identity to an amino acid sequence of SEQ ID NO:4148. In one aspect, provided herein is an antibody that binds TRGDC, comprising a heavy chain having an amino acid sequence having at least 95% identity to an amino acid sequence of SEQ ID NO:4147, and a light chain having an amino acid sequence having at least 95% identity to an amino acid sequence of SEQ ID NO:4148.

In one aspect, provided herein is an antibody that binds TRGDC, comprising: (i) a VH comprising a VH CDR1, a VH CDR2, and a VH CDR3 having an amino acid sequence of SEQ ID NOs:4149, 4150, and 4151, respectively, and (ii) a VL comprising a VL CDR1, VL CDR2, and VL CDR3 having an amino acid sequence of SEQ ID NOs:4152, 4153, and 4154, respectively. In one aspect, provided herein is an antibody that binds TRGDC, comprising: (i) a VH comprising a VH CDR1, a VH CDR2, and a VH CDR3 having an amino acid sequence of SEQ ID NOs:4155, 4156, and 4157, respectively, and (ii) a VL comprising a VL CDR1, VL CDR2, and VL CDR3 having an amino acid sequence of SEQ ID NOs:4158, 4159, and 4160, respectively. In one aspect, provided herein is an antibody that binds TRGDC, comprising: (i) a VH comprising a VH CDR1, a VH CDR2, and a VH CDR3 having an amino acid sequence of SEQ ID NOs:4161, 4162, and 4163, respectively, and (ii) a VL comprising a VL CDR1, VL CDR2, and VL CDR3 having an amino acid sequence of SEQ ID NOs:4164, 4165, and 4166, respectively. In one aspect, provided herein is an antibody that binds TRGDC, comprising: (i) a VH comprising a VH CDR1, a VH CDR2, and a VH CDR3 having an amino acid sequence of SEQ ID NOs:4167, 4168, and 4169, respectively, and (ii) a VL comprising a VL CDR1, VL CDR2, and VL CDR3 having an amino acid sequence of SEQ ID NOs:4170, 4171, and 4172, respectively. In one aspect, provided herein is an antibody that binds TRGDC, comprising: (i) a VH comprising a VH CDR1, a VH CDR2, and a VH CDR3 having an amino acid sequence of SEQ ID NOs:4173, 4174, and 4175, respectively, and (ii) a VL comprising a VL CDR1, VL CDR2, and VL CDR3 having an amino acid sequence of SEQ ID NOs:4176, 4177, and 4178, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4179; and (ii) a VL comprising a VL CDR1, a VL CDR2, and a VL CDR3 having an amino acid sequence of a VL CDR1, a VL CDR2, and a VL CDR3, respectively, of SEQ ID NO:4180. In one aspect, provided herein is an antibody that binds TRGDC, comprising a VH having an amino acid sequence of SEQ ID NO:4179. In one aspect, provided herein is an antibody that binds TRGDC, comprising a VL having an amino acid sequence of SEQ ID NO:4180. In one aspect, provided herein is an antibody that binds TRGDC, comprising a VH having an amino acid sequence of SEQ ID NO:4179, and a VL having an amino acid sequence of SEQ ID NO:4180. In one aspect, provided herein is an antibody that binds TRGDC, comprising a heavy chain having an amino acid sequence of SEQ ID NO:4181. In one aspect, provided herein is an antibody that binds TRGDC, comprising a light chain having an amino acid sequence of SEQ ID NO:4182. In one aspect, provided herein is an antibody that binds TRGDC, comprising a heavy chain having an amino acid sequence of SEQ ID NO:4181, and a light chain having an amino acid sequence of SEQ ID NO:4182. In one aspect, provided herein is an antibody that binds TRGDC, comprising a VH having an amino acid sequence having at least 95% identity to an amino acid sequence of SEQ ID NO:4179. In one aspect, provided herein is an antibody that binds TRGDC, comprising a VL having an amino acid sequence having at least 95% identity to an amino acid sequence of SEQ ID NO:4180. In one aspect, provided herein is an antibody that binds TRGDC, comprising a VH having an amino acid sequence having at least 95% identity to an amino acid sequence of SEQ ID NO:4179, and a VL having an amino acid sequence having at least 95% identity to an amino acid sequence of SEQ ID NO:4180. In one aspect, provided herein is an antibody that binds TRGDC, comprising a heavy chain having an amino acid sequence having at least 95% identity to an amino acid sequence of SEQ ID NO:4181. In one aspect, provided herein is an antibody that binds TRGDC, comprising a light chain having an amino acid sequence having at least 95% identity to an amino acid sequence of SEQ ID NO:4182. In one aspect, provided herein is an antibody that binds TRGDC, comprising a heavy chain having an amino acid sequence having at least 95% identity to an amino acid sequence of SEQ ID NO:4181, and a light chain having an amino acid sequence having at least 95% identity to an amino acid sequence of SEQ ID NO:4182.

In one aspect, provided herein is an antibody that binds TRGDC, comprising: (i) a VH comprising a VH CDR1, a VH CDR2, and a VH CDR3 having an amino acid sequence of SEQ ID NOs:4183, 4184, and 4185, respectively, and (ii) a VL comprising a VL CDR1, VL CDR2, and VL CDR3 having an amino acid sequence of SEQ ID NOs:4186, 4187, and 4188, respectively. In one aspect, provided herein is an antibody that binds TRGDC, comprising: (i) a VH comprising a VH CDR1, a VH CDR2, and a VH CDR3 having an amino acid sequence of SEQ ID NOs:4189, 4190, and 4191, respectively, and (ii) a VL comprising a VL CDR1, VL CDR2, and VL CDR3 having an amino acid sequence of SEQ ID NOs:4192, 4193, and 4194, respectively. In one aspect, provided herein is an antibody that binds TRGDC, comprising: (i) a VH comprising a VH CDR1, a VH CDR2, and a VH CDR3 having an amino acid sequence of SEQ ID NOs:4195, 4196, and 4197, respectively, and (ii) a VL comprising a VL CDR1, VL CDR2, and VL CDR3 having an amino acid sequence of SEQ ID NOs:4198, 4199, and 4200, respectively. In one aspect, provided herein is an antibody that binds TRGDC, comprising: (i) a VH comprising a VH CDR1, a VH CDR2, and a VH CDR3 having an amino acid sequence of SEQ ID NOs:4201, 4202, and 4203, respectively, and (ii) a VL comprising a VL CDR1, VL CDR2, and VL CDR3 having an amino acid sequence of SEQ ID NOs:4204, 4205, and 4206, respectively. In one aspect, provided herein is an antibody that binds TRGDC, comprising: (i) a VH comprising a VH CDR1, a VH CDR2, and a VH CDR3 having an amino acid sequence of SEQ ID NOs:4207, 4208, and 4209, respectively, and (ii) a VL comprising a VL CDR1, VL CDR2, and VL CDR3 having an amino acid sequence of SEQ ID NOs:4210, 4211, and 4212, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4213; and (ii) a VL comprising a VL CDR1, a VL CDR2, and a VL CDR3 having an amino acid sequence of a VL CDR1, a VL CDR2, and a VL CDR3, respectively, of SEQ ID NO:4214. In one aspect, provided herein is an antibody that binds TRGDC, comprising a VH having an amino acid sequence of SEQ ID NO:4213. In one aspect, provided herein is an antibody that binds TRGDC, comprising a VL having an amino acid sequence of SEQ ID NO:4214. In one aspect, provided herein is an antibody that binds TRGDC, comprising a VH having an amino acid sequence of SEQ ID NO:4213, and a VL having an amino acid sequence of SEQ ID NO:4214. In one aspect, provided herein is an antibody that binds TRGDC, comprising a heavy chain having an amino acid sequence of SEQ ID NO:4215. In one aspect, provided herein is an antibody that binds TRGDC, comprising a light chain having an amino acid sequence of SEQ ID NO:4216. In one aspect, provided herein is an antibody that binds TRGDC, comprising a heavy chain having an amino acid sequence of SEQ ID NO:4215, and a light chain having an amino acid sequence of SEQ ID NO:4216. In one aspect, provided herein is an antibody that binds TRGDC, comprising a VH having an amino acid sequence having at least 95% identity to an amino acid sequence of SEQ ID NO:4213. In one aspect, provided herein is an antibody that binds TRGDC, comprising a VL having an amino acid sequence having at least 95% identity to an amino acid sequence of SEQ ID NO:4214. In one aspect, provided herein is an antibody that binds TRGDC, comprising a VH having an amino acid sequence having at least 95% identity to an amino acid sequence of SEQ ID NO:4213, and a VL having an amino acid sequence having at least 95% identity to an amino acid sequence of SEQ ID NO:4214. In one aspect, provided herein is an antibody that binds TRGDC, comprising a heavy chain having an amino acid sequence having at least 95% identity to an amino acid sequence of SEQ ID NO:4215. In one aspect, provided herein is an antibody that binds TRGDC, comprising a light chain having an amino acid sequence having at least 95% identity to an amino acid sequence of SEQ ID NO:4216. In one aspect, provided herein is an antibody that binds TRGDC, comprising a heavy chain having an amino acid sequence having at least 95% identity to an amino acid sequence of SEQ ID NO:4215, and a light chain having an amino acid sequence having at least 95% identity to an amino acid sequence of SEQ ID NO:4216.

In one aspect, provided herein is an antibody that binds TRGDC, comprising: (i) a VH comprising a VH CDR1, a VH CDR2, and a VH CDR3 having an amino acid sequence of SEQ ID NOs:4217, 4218, and 4219, respectively, and (ii) a VL comprising a VL CDR1, VL CDR2, and VL CDR3 having an amino acid sequence of SEQ ID NOs:4220, 4221, and 4222, respectively. In one aspect, provided herein is an antibody that binds TRGDC, comprising: (i) a VH comprising a VH CDR1, a VH CDR2, and a VH CDR3 having an amino acid sequence of SEQ ID NOs:4223, 4224, and 4225, respectively, and (ii) a VL comprising a VL CDR1, VL CDR2, and VL CDR3 having an amino acid sequence of SEQ ID NOs:4226, 4227, and 4228, respectively. In one aspect, provided herein is an antibody that binds TRGDC, comprising: (i) a VH comprising a VH CDR1, a VH CDR2, and a VH CDR3 having an amino acid sequence of SEQ ID NOs:4229, 4230, and 4231, respectively, and (ii) a VL comprising a VL CDR1, VL CDR2, and VL CDR3 having an amino acid sequence of SEQ ID NOs:4232, 4233, and 4234, respectively. In one aspect, provided herein is an antibody that binds TRGDC, comprising: (i) a VH comprising a VH CDR1, a VH CDR2, and a VH CDR3 having an amino acid sequence of SEQ ID NOs:4235, 4236, and 4237, respectively, and (ii) a VL comprising a VL CDR1, VL CDR2, and VL CDR3 having an amino acid sequence of SEQ ID NOs:4238, 4239, and 4240, respectively. In one aspect, provided herein is an antibody that binds TRGDC, comprising: (i) a VH comprising a VH CDR1, a VH CDR2, and a VH CDR3 having an amino acid sequence of SEQ ID NOs:4241, 4242, and 4243, respectively, and (ii) a VL comprising a VL CDR1, VL CDR2, and VL CDR3 having an amino acid sequence of SEQ ID NOs:4244, 4245, and 4246, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4247; and (ii) a VL comprising a VL CDR1, a VL CDR2, and a VL CDR3 having an amino acid sequence of a VL CDR1, a VL CDR2, and a VL CDR3, respectively, of SEQ ID NO:4248. In one aspect, provided herein is an antibody that binds TRGDC, comprising a VH having an amino acid sequence of SEQ ID NO:4247. In one aspect, provided herein is an antibody that binds TRGDC, comprising a VL having an amino acid sequence of SEQ ID NO:4248. In one aspect, provided herein is an antibody that binds TRGDC, comprising a VH having an amino acid sequence of SEQ ID NO:4247, and a VL having an amino acid sequence of SEQ ID NO:4248. In one aspect, provided herein is an antibody that binds TRGDC, comprising a heavy chain having an amino acid sequence of SEQ ID NO:4249. In one aspect, provided herein is an antibody that binds TRGDC, comprising a light chain having an amino acid sequence of SEQ ID NO:4250. In one aspect, provided herein is an antibody that binds TRGDC, comprising a heavy chain having an amino acid sequence of SEQ ID NO:4249, and a light chain having an amino acid sequence of SEQ ID NO:4250. In one aspect, provided herein is an antibody that binds TRGDC, comprising a VH having an amino acid sequence having at least 95% identity to an amino acid sequence of SEQ ID NO:4247. In one aspect, provided herein is an antibody that binds TRGDC, comprising a VL having an amino acid sequence having at least 95% identity to an amino acid sequence of SEQ ID NO:4248. In one aspect, provided herein is an antibody that binds TRGDC, comprising a VH having an amino acid sequence having at least 95% identity to an amino acid sequence of SEQ ID NO:4247, and a VL having an amino acid sequence having at least 95% identity to an amino acid sequence of SEQ ID NO:4248. In one aspect, provided herein is an antibody that binds TRGDC, comprising a heavy chain having an amino acid sequence having at least 95% identity to an amino acid sequence of SEQ ID NO:4249. In one aspect, provided herein is an antibody that binds TRGDC, comprising a light chain having an amino acid sequence having at least 95% identity to an amino acid sequence of SEQ ID NO:4250. In one aspect, provided herein is an antibody that binds TRGDC, comprising a heavy chain having an amino acid sequence having at least 95% identity to an amino acid sequence of SEQ ID NO:4249, and a light chain having an amino acid sequence having at least 95% identity to an amino acid sequence of SEQ ID NO:4250.

In one aspect, provided herein is an antibody that binds TRGDC, comprising: (i) a VH comprising a VH CDR1, a VH CDR2, and a VH CDR3 having an amino acid sequence of SEQ ID NOs:4251, 4252, and 4253, respectively, and (ii) a VL comprising a VL CDR1, VL CDR2, and VL CDR3 having an amino acid sequence of SEQ ID NOs:4254, 4255, and 4256, respectively. In one aspect, provided herein is an antibody that binds TRGDC, comprising: (i) a VH comprising a VH CDR1, a VH CDR2, and a VH CDR3 having an amino acid sequence of SEQ ID NOs:4257, 4258, and 4259, respectively, and (ii) a VL comprising a VL CDR1, VL CDR2, and VL CDR3 having an amino acid sequence of SEQ ID NOs:4260, 4261, and 4262, respectively. In one aspect, provided herein is an antibody that binds TRGDC, comprising: (i) a VH comprising a VH CDR1, a VH CDR2, and a VH CDR3 having an amino acid sequence of SEQ ID NOs:4263, 4264, and 4265, respectively, and (ii) a VL comprising a VL CDR1, VL CDR2, and VL CDR3 having an amino acid sequence of SEQ ID NOs:4266, 4267, and 4268, respectively. In one aspect, provided herein is an antibody that binds TRGDC, comprising: (i) a VH comprising a VH CDR1, a VH CDR2, and a VH CDR3 having an amino acid sequence of SEQ ID NOs:4269, 4270, and 4271, respectively, and (ii) a VL comprising a VL CDR1, VL CDR2, and VL CDR3 having an amino acid sequence of SEQ ID NOs:4272, 4273, and 4274, respectively. In one aspect, provided herein is an antibody that binds TRGDC, comprising: (i) a VH comprising a VH CDR1, a VH CDR2, and a VH CDR3 having an amino acid sequence of SEQ ID NOs:4275, 4276, and 4277, respectively, and (ii) a VL comprising a VL CDR1, VL CDR2, and VL CDR3 having an amino acid sequence of SEQ ID NOs:4278, 4279, and 4280, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4281; and (ii) a VL comprising a VL CDR1, a VL CDR2, and a VL CDR3 having an amino acid sequence of a VL CDR1, a VL CDR2, and a VL CDR3, respectively, of SEQ ID NO:4282. In one aspect, provided herein is an antibody that binds TRGDC, comprising a VH having an amino acid sequence of SEQ ID NO:4281. In one aspect, provided herein is an antibody that binds TRGDC, comprising a VL having an amino acid sequence of SEQ ID NO:4282. In one aspect, provided herein is an antibody that binds TRGDC, comprising a VH having an amino acid sequence of SEQ ID NO:4281, and a VL having an amino acid sequence of SEQ ID NO:4282. In one aspect, provided herein is an antibody that binds TRGDC, comprising a heavy chain having an amino acid sequence of SEQ ID NO:4283. In one aspect, provided herein is an antibody that binds TRGDC, comprising a light chain having an amino acid sequence of SEQ ID NO:4284. In one aspect, provided herein is an antibody that binds TRGDC, comprising a heavy chain having an amino acid sequence of SEQ ID NO:4283, and a light chain having an amino acid sequence of SEQ ID NO:4284. In one aspect, provided herein is an antibody that binds TRGDC, comprising a VH having an amino acid sequence having at least 95% identity to an amino acid sequence of SEQ ID NO:4281. In one aspect, provided herein is an antibody that binds TRGDC, comprising a VL having an amino acid sequence having at least 95% identity to an amino acid sequence of SEQ ID NO:4282. In one aspect, provided herein is an antibody that binds TRGDC, comprising a VH having an amino acid sequence having at least 95% identity to an amino acid sequence of SEQ ID NO:4281, and a VL having an amino acid sequence having at least 95% identity to an amino acid sequence of SEQ ID NO:4282. In one aspect, provided herein is an antibody that binds TRGDC, comprising a heavy chain having an amino acid sequence having at least 95% identity to an amino acid sequence of SEQ ID NO:4283. In one aspect, provided herein is an antibody that binds TRGDC, comprising a light chain having an amino acid sequence having at least 95% identity to an amino acid sequence of SEQ ID NO:4284. In one aspect, provided herein is an antibody that binds TRGDC, comprising a heavy chain having an amino acid sequence having at least 95% identity to an amino acid sequence of SEQ ID NO:4283, and a light chain having an amino acid sequence having at least 95% identity to an amino acid sequence of SEQ ID NO:4284.

In one aspect, provided herein is an antibody that binds TRGDC, comprising: (i) a VH comprising a VH CDR1, a VH CDR2, and a VH CDR3 having an amino acid sequence of SEQ ID NOs:4285, 4286, and 4287, respectively, and (ii) a VL comprising a VL CDR1, VL CDR2, and VL CDR3 having an amino acid sequence of SEQ ID NOs:4288, 4289, and 4290, respectively. In one aspect, provided herein is an antibody that binds TRGDC, comprising: (i) a VH comprising a VH CDR1, a VH CDR2, and a VH CDR3 having an amino acid sequence of SEQ ID NOs:4291, 4292, and 4293, respectively, and (ii) a VL comprising a VL CDR1, VL CDR2, and VL CDR3 having an amino acid sequence of SEQ ID NOs:4294, 4295, and 4296, respectively. In one aspect, provided herein is an antibody that binds TRGDC, comprising: (i) a VH comprising a VH CDR1, a VH CDR2, and a VH CDR3 having an amino acid sequence of SEQ ID NOs:4297, 4298, and 4299, respectively, and (ii) a VL comprising a VL CDR1, VL CDR2, and VL CDR3 having an amino acid sequence of SEQ ID NOs:4300, 4301, and 4302, respectively. In one aspect, provided herein is an antibody that binds TRGDC, comprising: (i) a VH comprising a VH CDR1, a VH CDR2, and a VH CDR3 having an amino acid sequence of SEQ ID NOs:4303, 4304, and 4305, respectively, and (ii) a VL comprising a VL CDR1, VL CDR2, and VL CDR3 having an amino acid sequence of SEQ ID NOs:4306, 4307, and 4308, respectively. In one aspect, provided herein is an antibody that binds TRGDC, comprising: (i) a VH comprising a VH CDR1, a VH CDR2, and a VH CDR3 having an amino acid sequence of SEQ ID NOs:4309, 4310, and 4311, respectively, and (ii) a VL comprising a VL CDR1, VL CDR2, and VL CDR3 having an amino acid sequence of SEQ ID NOs:4312, 4313, and 4314, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4315; and (ii) a VL comprising a VL CDR1, a VL CDR2, and a VL CDR3 having an amino acid sequence of a VL CDR1, a VL CDR2, and a VL CDR3, respectively, of SEQ ID NO:4316. In one aspect, provided herein is an antibody that binds TRGDC, comprising a VH having an amino acid sequence of SEQ ID NO:4315. In one aspect, provided herein is an antibody that binds TRGDC, comprising a VL having an amino acid sequence of SEQ ID NO:4316. In one aspect, provided herein is an antibody that binds TRGDC, comprising a VH having an amino acid sequence of SEQ ID NO:4315, and a VL having an amino acid sequence of SEQ ID NO:4316. In one aspect, provided herein is an antibody that binds TRGDC, comprising a heavy chain having an amino acid sequence of SEQ ID NO:4317. In one aspect, provided herein is an antibody that binds TRGDC, comprising a light chain having an amino acid sequence of SEQ ID NO:4318. In one aspect, provided herein is an antibody that binds TRGDC, comprising a heavy chain having an amino acid sequence of SEQ ID NO:4317, and a light chain having an amino acid sequence of SEQ ID NO:4318. In one aspect, provided herein is an antibody that binds TRGDC, comprising a VH having an amino acid sequence having at least 95% identity to an amino acid sequence of SEQ ID NO:4315. In one aspect, provided herein is an antibody that binds TRGDC, comprising a VL having an amino acid sequence having at least 95% identity to an amino acid sequence of SEQ ID NO:4316. In one aspect, provided herein is an antibody that binds TRGDC, comprising a VH having an amino acid sequence having at least 95% identity to an amino acid sequence of SEQ ID NO:4315, and a VL having an amino acid sequence having at least 95% identity to an amino acid sequence of SEQ ID NO:4316. In one aspect, provided herein is an antibody that binds TRGDC, comprising a heavy chain having an amino acid sequence having at least 95% identity to an amino acid sequence of SEQ ID NO:4317. In one aspect, provided herein is an antibody that binds TRGDC, comprising a light chain having an amino acid sequence having at least 95% identity to an amino acid sequence of SEQ ID NO:4318. In one aspect, provided herein is an antibody that binds TRGDC, comprising a heavy chain having an amino acid sequence having at least 95% identity to an amino acid sequence of SEQ ID NO:4317, and a light chain having an amino acid sequence having at least 95% identity to an amino acid sequence of SEQ ID NO:4318.

In one aspect, provided herein is an antibody that binds TRGDC, comprising: (i) a VH comprising a VH CDR1, a VH CDR2, and a VH CDR3 having an amino acid sequence of SEQ ID NOs:4319, 4320, and 4321, respectively, and (ii) a VL comprising a VL CDR1, VL CDR2, and VL CDR3 having an amino acid sequence of SEQ ID NOs:4322, 4323, and 4324, respectively. In one aspect, provided herein is an antibody that binds TRGDC, comprising: (i) a VH comprising a VH CDR1, a VH CDR2, and a VH CDR3 having an amino acid sequence of SEQ ID NOs:4325, 4326, and 4327, respectively, and (ii) a VL comprising a VL CDR1, VL CDR2, and VL CDR3 having an amino acid sequence of SEQ ID NOs:4328, 4329, and 4330, respectively. In one aspect, provided herein is an antibody that binds TRGDC, comprising: (i) a VH comprising a VH CDR1, a VH CDR2, and a VH CDR3 having an amino acid sequence of SEQ ID NOs:4331, 4332, and 4333, respectively, and (ii) a VL comprising a VL CDR1, VL CDR2, and VL CDR3 having an amino acid sequence of SEQ ID NOs:4334, 4335, and 4336, respectively. In one aspect, provided herein is an antibody that binds TRGDC, comprising: (i) a VH comprising a VH CDR1, a VH CDR2, and a VH CDR3 having an amino acid sequence of SEQ ID NOs:4337, 4338, and 4339, respectively, and (ii) a VL comprising a VL CDR1, VL CDR2, and VL CDR3 having an amino acid sequence of SEQ ID NOs:4340, 4341, and 4342, respectively. In one aspect, provided herein is an antibody that binds TRGDC, comprising: (i) a VH comprising a VH CDR1, a VH CDR2, and a VH CDR3 having an amino acid sequence of SEQ ID NOs:4343, 4344, and 4345, respectively, and (ii) a VL comprising a VL CDR1, VL CDR2, and VL CDR3 having an amino acid sequence of SEQ ID NOs:4346, 4347, and 4348, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4349; and (ii) a VL comprising a VL CDR1, a VL CDR2, and a VL CDR3 having an amino acid sequence of a VL CDR1, a VL CDR2, and a VL CDR3, respectively, of SEQ ID NO:4350. In one aspect, provided herein is an antibody that binds TRGDC, comprising a VH having an amino acid sequence of SEQ ID NO:4349. In one aspect, provided herein is an antibody that binds TRGDC, comprising a VL having an amino acid sequence of SEQ ID NO:4350. In one aspect, provided herein is an antibody that binds TRGDC, comprising a VH having an amino acid sequence of SEQ ID NO:4349, and a VL having an amino acid sequence of SEQ ID NO:4350. In one aspect, provided herein is an antibody that binds TRGDC, comprising a heavy chain having an amino acid sequence of SEQ ID NO:4351. In one aspect, provided herein is an antibody that binds TRGDC, comprising a light chain having an amino acid sequence of SEQ ID NO:4352. In one aspect, provided herein is an antibody that binds TRGDC, comprising a heavy chain having an amino acid sequence of SEQ ID NO:4351, and a light chain having an amino acid sequence of SEQ ID NO:4352. In one aspect, provided herein is an antibody that binds TRGDC, comprising a VH having an amino acid sequence having at least 95% identity to an amino acid sequence of SEQ ID NO:4349. In one aspect, provided herein is an antibody that binds TRGDC, comprising a VL having an amino acid sequence having at least 95% identity to an amino acid sequence of SEQ ID NO:4350. In one aspect, provided herein is an antibody that binds TRGDC, comprising a VH having an amino acid sequence having at least 95% identity to an amino acid sequence of SEQ ID NO:4349, and a VL having an amino acid sequence having at least 95% identity to an amino acid sequence of SEQ ID NO:4350. In one aspect, provided herein is an antibody that binds TRGDC, comprising a heavy chain having an amino acid sequence having at least 95% identity to an amino acid sequence of SEQ ID NO:4351. In one aspect, provided herein is an antibody that binds TRGDC, comprising a light chain having an amino acid sequence having at least 95% identity to an amino acid sequence of SEQ ID NO:4352. In one aspect, provided herein is an antibody that binds TRGDC, comprising a heavy chain having an amino acid sequence having at least 95% identity to an amino acid sequence of SEQ ID NO:4351, and a light chain having an amino acid sequence having at least 95% identity to an amino acid sequence of SEQ ID NO:4352.

In one aspect, provided herein is an antibody that binds TRGDC, comprising: (i) a VH comprising a VH CDR1, a VH CDR2, and a VH CDR3 having an amino acid sequence of SEQ ID NOs:4353, 4354, and 4355, respectively, and (ii) a VL comprising a VL CDR1, VL CDR2, and VL CDR3 having an amino acid sequence of SEQ ID NOs:4356, 4357, and 4358, respectively. In one aspect, provided herein is an antibody that binds TRGDC, comprising: (i) a VH comprising a VH CDR1, a VH CDR2, and a VH CDR3 having an amino acid sequence of SEQ ID NOs:4359, 4360, and 4361, respectively, and (ii) a VL comprising a VL CDR1, VL CDR2, and VL CDR3 having an amino acid sequence of SEQ ID NOs:4362, 4363, and 4364, respectively. In one aspect, provided herein is an antibody that binds TRGDC, comprising: (i) a VH comprising a VH CDR1, a VH CDR2, and a VH CDR3 having an amino acid sequence of SEQ ID NOs:4365, 4366, and 4367, respectively, and (ii) a VL comprising a VL CDR1, VL CDR2, and VL CDR3 having an amino acid sequence of SEQ ID NOs:4368, 4369, and 4370, respectively. In one aspect, provided herein is an antibody that binds TRGDC, comprising: (i) a VH comprising a VH CDR1, a VH CDR2, and a VH CDR3 having an amino acid sequence of SEQ ID NOs:4371, 4372, and 4373, respectively, and (ii) a VL comprising a VL CDR1, VL CDR2, and VL CDR3 having an amino acid sequence of SEQ ID NOs:4374, 4375, and 4376, respectively. In one aspect, provided herein is an antibody that binds TRGDC, comprising: (i) a VH comprising a VH CDR1, a VH CDR2, and a VH CDR3 having an amino acid sequence of SEQ ID NOs:4377, 4378, and 4379, respectively, and (ii) a VL comprising a VL CDR1, VL CDR2, and VL CDR3 having an amino acid sequence of SEQ ID NOs:4380, 4381, and 4382, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4383; and (ii) a VL comprising a VL CDR1, a VL CDR2, and a VL CDR3 having an amino acid sequence of a VL CDR1, a VL CDR2, and a VL CDR3, respectively, of SEQ ID NO:4384. In one aspect, provided herein is an antibody that binds TRGDC, comprising a VH having an amino acid sequence of SEQ ID NO:4383. In one aspect, provided herein is an antibody that binds TRGDC, comprising a VL having an amino acid sequence of SEQ ID NO:4384. In one aspect, provided herein is an antibody that binds TRGDC, comprising a VH having an amino acid sequence of SEQ ID NO:4383, and a VL having an amino acid sequence of SEQ ID NO:4384. In one aspect, provided herein is an antibody that binds TRGDC, comprising a heavy chain having an amino acid sequence of SEQ ID NO:4385. In one aspect, provided herein is an antibody that binds TRGDC, comprising a light chain having an amino acid sequence of SEQ ID NO:4386. In one aspect, provided herein is an antibody that binds TRGDC, comprising a heavy chain having an amino acid sequence of SEQ ID NO:4385, and a light chain having an amino acid sequence of SEQ ID NO:4386. In one aspect, provided herein is an antibody that binds TRGDC, comprising a VH having an amino acid sequence having at least 95% identity to an amino acid sequence of SEQ ID NO:4383. In one aspect, provided herein is an antibody that binds TRGDC, comprising a VL having an amino acid sequence having at least 95% identity to an amino acid sequence of SEQ ID NO:4384. In one aspect, provided herein is an antibody that binds TRGDC, comprising a VH having an amino acid sequence having at least 95% identity to an amino acid sequence of SEQ ID NO:4383, and a VL having an amino acid sequence having at least 95% identity to an amino acid sequence of SEQ ID NO:4384. In one aspect, provided herein is an antibody that binds TRGDC, comprising a heavy chain having an amino acid sequence having at least 95% identity to an amino acid sequence of SEQ ID NO:4385. In one aspect, provided herein is an antibody that binds TRGDC, comprising a light chain having an amino acid sequence having at least 95% identity to an amino acid sequence of SEQ ID NO:4386. In one aspect, provided herein is an antibody that binds TRGDC, comprising a heavy chain having an amino acid sequence having at least 95% identity to an amino acid sequence of SEQ ID NO:4385, and a light chain having an amino acid sequence having at least 95% identity to an amino acid sequence of SEQ ID NO:4386.

In one aspect, provided herein is an antibody that binds TRGDC, comprising: (i) a VH comprising a VH CDR1, a VH CDR2, and a VH CDR3 having an amino acid sequence of SEQ ID NOs:4387, 4388, and 4389, respectively, and (ii) a VL comprising a VL CDR1, VL CDR2, and VL CDR3 having an amino acid sequence of SEQ ID NOs:4390, 4391, and 4392, respectively. In one aspect, provided herein is an antibody that binds TRGDC, comprising: (i) a VH comprising a VH CDR1, a VH CDR2, and a VH CDR3 having an amino acid sequence of SEQ ID NOs:4393, 4394, and 4395, respectively, and (ii) a VL comprising a VL CDR1, VL CDR2, and VL CDR3 having an amino acid sequence of SEQ ID NOs:4396, 4397, and 4398, respectively. In one aspect, provided herein is an antibody that binds TRGDC, comprising: (i) a VH comprising a VH CDR1, a VH CDR2, and a VH CDR3 having an amino acid sequence of SEQ ID NOs:4399, 4400, and 4401, respectively, and (ii) a VL comprising a VL CDR1, VL CDR2, and VL CDR3 having an amino acid sequence of SEQ ID NOs:4402, 4403, and 4404, respectively. In one aspect, provided herein is an antibody that binds TRGDC, comprising: (i) a VH comprising a VH CDR1, a VH CDR2, and a VH CDR3 having an amino acid sequence of SEQ ID NOs:4405, 4406, and 4407, respectively, and (ii) a VL comprising a VL CDR1, VL CDR2, and VL CDR3 having an amino acid sequence of SEQ ID NOs:4408, 4409, and 4410, respectively. In one aspect, provided herein is an antibody that binds TRGDC, comprising: (i) a VH comprising a VH CDR1, a VH CDR2, and a VH CDR3 having an amino acid sequence of SEQ ID NOs:4411, 4412, and 4413, respectively, and (ii) a VL comprising a VL CDR1, VL CDR2, and VL CDR3 having an amino acid sequence of SEQ ID NOs:4414, 4415, and 4416, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4417; and (ii) a VL comprising a VL CDR1, a VL CDR2, and a VL CDR3 having an amino acid sequence of a VL CDR1, a VL CDR2, and a VL CDR3, respectively, of SEQ ID NO:4418. In one aspect, provided herein is an antibody that binds TRGDC, comprising a VH having an amino acid sequence of SEQ ID NO:4417. In one aspect, provided herein is an antibody that binds TRGDC, comprising a VL having an amino acid sequence of SEQ ID NO:4418. In one aspect, provided herein is an antibody that binds TRGDC, comprising a VH having an amino acid sequence of SEQ ID NO:4417, and a VL having an amino acid sequence of SEQ ID NO:4418. In one aspect, provided herein is an antibody that binds TRGDC, comprising a heavy chain having an amino acid sequence of SEQ ID NO:4419. In one aspect, provided herein is an antibody that binds TRGDC, comprising a light chain having an amino acid sequence of SEQ ID NO:4420. In one aspect, provided herein is an antibody that binds TRGDC, comprising a heavy chain having an amino acid sequence of SEQ ID NO:4419, and a light chain having an amino acid sequence of SEQ ID NO:4420. In one aspect, provided herein is an antibody that binds TRGDC, comprising a VH having an amino acid sequence having at least 95% identity to an amino acid sequence of SEQ ID NO:4417. In one aspect, provided herein is an antibody that binds TRGDC, comprising a VL having an amino acid sequence having at least 95% identity to an amino acid sequence of SEQ ID NO:4418. In one aspect, provided herein is an antibody that binds TRGDC, comprising a VH having an amino acid sequence having at least 95% identity to an amino acid sequence of SEQ ID NO:4417, and a VL having an amino acid sequence having at least 95% identity to an amino acid sequence of SEQ ID NO:4418. In one aspect, provided herein is an antibody that binds TRGDC, comprising a heavy chain having an amino acid sequence having at least 95% identity to an amino acid sequence of SEQ ID NO:4419. In one aspect, provided herein is an antibody that binds TRGDC, comprising a light chain having an amino acid sequence having at least 95% identity to an amino acid sequence of SEQ ID NO:4420. In one aspect, provided herein is an antibody that binds TRGDC, comprising a heavy chain having an amino acid sequence having at least 95% identity to an amino acid sequence of SEQ ID NO:4419, and a light chain having an amino acid sequence having at least 95% identity to an amino acid sequence of SEQ ID NO:4420.

In one aspect, provided herein is an antibody that binds TRGDC, comprising: (i) a VH comprising a VH CDR1, a VH CDR2, and a VH CDR3 having an amino acid sequence of SEQ ID NOs:4421, 4422, and 4423, respectively, and (ii) a VL comprising a VL CDR1, VL CDR2, and VL CDR3 having an amino acid sequence of SEQ ID NOs:4424, 4425, and 4426, respectively. In one aspect, provided herein is an antibody that binds TRGDC, comprising: (i) a VH comprising a VH CDR1, a VH CDR2, and a VH CDR3 having an amino acid sequence of SEQ ID NOs:4427, 4428, and 4429, respectively, and (ii) a VL comprising a VL CDR1, VL CDR2, and VL CDR3 having an amino acid sequence of SEQ ID NOs:4430, 4431, and 4432, respectively. In one aspect, provided herein is an antibody that binds TRGDC, comprising: (i) a VH comprising a VH CDR1, a VH CDR2, and a VH CDR3 having an amino acid sequence of SEQ ID NOs:4433, 4434, and 4435, respectively, and (ii) a VL comprising a VL CDR1, VL CDR2, and VL CDR3 having an amino acid sequence of SEQ ID NOs:4436, 4437, and 4438, respectively. In one aspect, provided herein is an antibody that binds TRGDC, comprising: (i) a VH comprising a VH CDR1, a VH CDR2, and a VH CDR3 having an amino acid sequence of SEQ ID NOs:4439, 4440, and 4441, respectively, and (ii) a VL comprising a VL CDR1, VL CDR2, and VL CDR3 having an amino acid sequence of SEQ ID NOs:4442, 4443, and 4444, respectively. In one aspect, provided herein is an antibody that binds TRGDC, comprising: (i) a VH comprising a VH CDR1, a VH CDR2, and a VH CDR3 having an amino acid sequence of SEQ ID NOs:4445, 4446, and 4447, respectively, and (ii) a VL comprising a VL CDR1, VL CDR2, and VL CDR3 having an amino acid sequence of SEQ ID NOs:4448, 4449, and 4450, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4451; and (ii) a VL comprising a VL CDR1, a VL CDR2, and a VL CDR3 having an amino acid sequence of a VL CDR1, a VL CDR2, and a VL CDR3, respectively, of SEQ ID NO:4452. In one aspect, provided herein is an antibody that binds TRGDC, comprising a VH having an amino acid sequence of SEQ ID NO:4451. In one aspect, provided herein is an antibody that binds TRGDC, comprising a VL having an amino acid sequence of SEQ ID NO:4452. In one aspect, provided herein is an antibody that binds TRGDC, comprising a VH having an amino acid sequence of SEQ ID NO:4451, and a VL having an amino acid sequence of SEQ ID NO:4452. In one aspect, provided herein is an antibody that binds TRGDC, comprising a heavy chain having an amino acid sequence of SEQ ID NO:4453. In one aspect, provided herein is an antibody that binds TRGDC, comprising a light chain having an amino acid sequence of SEQ ID NO:4454. In one aspect, provided herein is an antibody that binds TRGDC, comprising a heavy chain having an amino acid sequence of SEQ ID NO:4453, and a light chain having an amino acid sequence of SEQ ID NO:4454. In one aspect, provided herein is an antibody that binds TRGDC, comprising a VH having an amino acid sequence having at least 95% identity to an amino acid sequence of SEQ ID NO:4451. In one aspect, provided herein is an antibody that binds TRGDC, comprising a VL having an amino acid sequence having at least 95% identity to an amino acid sequence of SEQ ID NO:4452. In one aspect, provided herein is an antibody that binds TRGDC, comprising a VH having an amino acid sequence having at least 95% identity to an amino acid sequence of SEQ ID NO:4451, and a VL having an amino acid sequence having at least 95% identity to an amino acid sequence of SEQ ID NO:4452. In one aspect, provided herein is an antibody that binds TRGDC, comprising a heavy chain having an amino acid sequence having at least 95% identity to an amino acid sequence of SEQ ID NO:4453. In one aspect, provided herein is an antibody that binds TRGDC, comprising a light chain having an amino acid sequence having at least 95% identity to an amino acid sequence of SEQ ID NO:4454. In one aspect, provided herein is an antibody that binds TRGDC, comprising a heavy chain having an amino acid sequence having at least 95% identity to an amino acid sequence of SEQ ID NO:4453, and a light chain having an amino acid sequence having at least 95% identity to an amino acid sequence of SEQ ID NO:4454.

In one aspect, provided herein is an antibody that binds TRGDC, comprising: (i) a VH comprising a VH CDR1, a VH CDR2, and a VH CDR3 having an amino acid sequence of SEQ ID NOs:4455, 4456, and 4457, respectively, and (ii) a VL comprising a VL CDR1, VL CDR2, and VL CDR3 having an amino acid sequence of SEQ ID NOs:4458, 4459, and 4460, respectively. In one aspect, provided herein is an antibody that binds TRGDC, comprising: (i) a VH comprising a VH CDR1, a VH CDR2, and a VH CDR3 having an amino acid sequence of SEQ ID NOs:4461, 4462, and 4463, respectively, and (ii) a VL comprising a VL CDR1, VL CDR2, and VL CDR3 having an amino acid sequence of SEQ ID NOs:4464, 4465, and 4466, respectively. In one aspect, provided herein is an antibody that binds TRGDC, comprising: (i) a VH comprising a VH CDR1, a VH CDR2, and a VH CDR3 having an amino acid sequence of SEQ ID NOs:4467, 4468, and 4469, respectively, and (ii) a VL comprising a VL CDR1, VL CDR2, and VL CDR3 having an amino acid sequence of SEQ ID NOs:4470, 4471, and 4472, respectively. In one aspect, provided herein is an antibody that binds TRGDC, comprising: (i) a VH comprising a VH CDR1, a VH CDR2, and a VH CDR3 having an amino acid sequence of SEQ ID NOs:4473, 4474, and 4475, respectively, and (ii) a VL comprising a VL CDR1, VL CDR2, and VL CDR3 having an amino acid sequence of SEQ ID NOs:4476, 4477, and 4478, respectively. In one aspect, provided herein is an antibody that binds TRGDC, comprising: (i) a VH comprising a VH CDR1, a VH CDR2, and a VH CDR3 having an amino acid sequence of SEQ ID NOs:4479, 4480, and 4481, respectively, and (ii) a VL comprising a VL CDR1, VL CDR2, and VL CDR3 having an amino acid sequence of SEQ ID NOs:4482, 4483, and 4484, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4485; and (ii) a VL comprising a VL CDR1, a VL CDR2, and a VL CDR3 having an amino acid sequence of a VL CDR1, a VL CDR2, and a VL CDR3, respectively, of SEQ ID NO:4486. In one aspect, provided herein is an antibody that binds TRGDC, comprising a VH having an amino acid sequence of SEQ ID NO:4485. In one aspect, provided herein is an antibody that binds TRGDC, comprising a VL having an amino acid sequence of SEQ ID NO:4486. In one aspect, provided herein is an antibody that binds TRGDC, comprising a VH having an amino acid sequence of SEQ ID NO:4485, and a VL having an amino acid sequence of SEQ ID NO:4486. In one aspect, provided herein is an antibody that binds TRGDC, comprising a heavy chain having an amino acid sequence of SEQ ID NO:4487. In one aspect, provided herein is an antibody that binds TRGDC, comprising a light chain having an amino acid sequence of SEQ ID NO:4488. In one aspect, provided herein is an antibody that binds TRGDC, comprising a heavy chain having an amino acid sequence of SEQ ID NO:4487, and a light chain having an amino acid sequence of SEQ ID NO:4488. In one aspect, provided herein is an antibody that binds TRGDC, comprising a VH having an amino acid sequence having at least 95% identity to an amino acid sequence of SEQ ID NO:4485. In one aspect, provided herein is an antibody that binds TRGDC, comprising a VL having an amino acid sequence having at least 95% identity to an amino acid sequence of SEQ ID NO:4486. In one aspect, provided herein is an antibody that binds TRGDC, comprising a VH having an amino acid sequence having at least 95% identity to an amino acid sequence of SEQ ID NO:4485, and a VL having an amino acid sequence having at least 95% identity to an amino acid sequence of SEQ ID NO:4486. In one aspect, provided herein is an antibody that binds TRGDC, comprising a heavy chain having an amino acid sequence having at least 95% identity to an amino acid sequence of SEQ ID NO:4487. In one aspect, provided herein is an antibody that binds TRGDC, comprising a light chain having an amino acid sequence having at least 95% identity to an amino acid sequence of SEQ ID NO:4488. In one aspect, provided herein is an antibody that binds TRGDC, comprising a heavy chain having an amino acid sequence having at least 95% identity to an amino acid sequence of SEQ ID NO:4487, and a light chain having an amino acid sequence having at least 95% identity to an amino acid sequence of SEQ ID NO:4488.

In one aspect, provided herein is an antibody that binds TRGDC, comprising: (i) a VH comprising a VH CDR1, a VH CDR2, and a VH CDR3 having an amino acid sequence of SEQ ID NOs:4489, 4490, and 4491, respectively, and (ii) a VL comprising a VL CDR1, VL CDR2, and VL CDR3 having an amino acid sequence of SEQ ID NOs:4492, 4493, and 4494, respectively. In one aspect, provided herein is an antibody that binds TRGDC, comprising: (i) a VH comprising a VH CDR1, a VH CDR2, and a VH CDR3 having an amino acid sequence of SEQ ID NOs:4495, 4496, and 4497, respectively, and (ii) a VL comprising a VL CDR1, VL CDR2, and VL CDR3 having an amino acid sequence of SEQ ID NOs:4498, 4499, and 4500, respectively. In one aspect, provided herein is an antibody that binds TRGDC, comprising: (i) a VH comprising a VH CDR1, a VH CDR2, and a VH CDR3 having an amino acid sequence of SEQ ID NOs:4501, 4502, and 4503, respectively, and (ii) a VL comprising a VL CDR1, VL CDR2, and VL CDR3 having an amino acid sequence of SEQ ID NOs:4504, 4505, and 4506, respectively. In one aspect, provided herein is an antibody that binds TRGDC, comprising: (i) a VH comprising a VH CDR1, a VH CDR2, and a VH CDR3 having an amino acid sequence of SEQ ID NOs:4507, 4508, and 4509, respectively, and (ii) a VL comprising a VL CDR1, VL CDR2, and VL CDR3 having an amino acid sequence of SEQ ID NOs:4510, 4511, and 4512, respectively. In one aspect, provided herein is an antibody that binds TRGDC, comprising: (i) a VH comprising a VH CDR1, a VH CDR2, and a VH CDR3 having an amino acid sequence of SEQ ID NOs:4513, 4514, and 4515, respectively, and (ii) a VL comprising a VL CDR1, VL CDR2, and VL CDR3 having an amino acid sequence of SEQ ID NOs:4516, 4517, and 4518, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4519; and (ii) a VL comprising a VL CDR1, a VL CDR2, and a VL CDR3 having an amino acid sequence of a VL CDR1, a VL CDR2, and a VL CDR3, respectively, of SEQ ID NO:4520. In one aspect, provided herein is an antibody that binds TRGDC, comprising a VH having an amino acid sequence of SEQ ID NO:4519. In one aspect, provided herein is an antibody that binds TRGDC, comprising a VL having an amino acid sequence of SEQ ID NO:4520. In one aspect, provided herein is an antibody that binds TRGDC, comprising a VH having an amino acid sequence of SEQ ID NO:4519, and a VL having an amino acid sequence of SEQ ID NO:4520. In one aspect, provided herein is an antibody that binds TRGDC, comprising a heavy chain having an amino acid sequence of SEQ ID NO:4521. In one aspect, provided herein is an antibody that binds TRGDC, comprising a light chain having an amino acid sequence of SEQ ID NO:4522. In one aspect, provided herein is an antibody that binds TRGDC, comprising a heavy chain having an amino acid sequence of SEQ ID NO:4521, and a light chain having an amino acid sequence of SEQ ID NO:4522. In one aspect, provided herein is an antibody that binds TRGDC, comprising a VH having an amino acid sequence having at least 95% identity to an amino acid sequence of SEQ ID NO:4519. In one aspect, provided herein is an antibody that binds TRGDC, comprising a VL having an amino acid sequence having at least 95% identity to an amino acid sequence of SEQ ID NO:4520. In one aspect, provided herein is an antibody that binds TRGDC, comprising a VH having an amino acid sequence having at least 95% identity to an amino acid sequence of SEQ ID NO:4519, and a VL having an amino acid sequence having at least 95% identity to an amino acid sequence of SEQ ID NO:4520. In one aspect, provided herein is an antibody that binds TRGDC, comprising a heavy chain having an amino acid sequence having at least 95% identity to an amino acid sequence of SEQ ID NO:4521. In one aspect, provided herein is an antibody that binds TRGDC, comprising a light chain having an amino acid sequence having at least 95% identity to an amino acid sequence of SEQ ID NO:4522. In one aspect, provided herein is an antibody that binds TRGDC, comprising a heavy chain having an amino acid sequence having at least 95% identity to an amino acid sequence of SEQ ID NO:4521, and a light chain having an amino acid sequence having at least 95% identity to an amino acid sequence of SEQ ID NO:4522.

In one aspect, provided herein is an antibody that binds TRGDC, comprising: (i) a VH comprising a VH CDR1, a VH CDR2, and a VH CDR3 having an amino acid sequence of SEQ ID NOs:4523, 4524, and 4525, respectively, and (ii) a VL comprising a VL CDR1, VL CDR2, and VL CDR3 having an amino acid sequence of SEQ ID NOs:4526, 4527, and 4528, respectively. In one aspect, provided herein is an antibody that binds TRGDC, comprising: (i) a VH comprising a VH CDR1, a VH CDR2, and a VH CDR3 having an amino acid sequence of SEQ ID NOs:4529, 4530, and 4531, respectively, and (ii) a VL comprising a VL CDR1, VL CDR2, and VL CDR3 having an amino acid sequence of SEQ ID NOs:4532, 4533, and 4534, respectively. In one aspect, provided herein is an antibody that binds TRGDC, comprising: (i) a VH comprising a VH CDR1, a VH CDR2, and a VH CDR3 having an amino acid sequence of SEQ ID NOs:4535, 4536, and 4537, respectively, and (ii) a VL comprising a VL CDR1, VL CDR2, and VL CDR3 having an amino acid sequence of SEQ ID NOs:4538, 4539, and 4540, respectively. In one aspect, provided herein is an antibody that binds TRGDC, comprising: (i) a VH comprising a VH CDR1, a VH CDR2, and a VH CDR3 having an amino acid sequence of SEQ ID NOs:4541, 4542, and 4543, respectively, and (ii) a VL comprising a VL CDR1, VL CDR2, and VL CDR3 having an amino acid sequence of SEQ ID NOs:4544, 4545, and 4546, respectively. In one aspect, provided herein is an antibody that binds TRGDC, comprising: (i) a VH comprising a VH CDR1, a VH CDR2, and a VH CDR3 having an amino acid sequence of SEQ ID NOs:4547, 4548, and 4549, respectively, and (ii) a VL comprising a VL CDR1, VL CDR2, and VL CDR3 having an amino acid sequence of SEQ ID NOs:4550, 4551, and 4552, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4553; and (ii) a VL comprising a VL CDR1, a VL CDR2, and a VL CDR3 having an amino acid sequence of a VL CDR1, a VL CDR2, and a VL CDR3, respectively, of SEQ ID NO:4554. In one aspect, provided herein is an antibody that binds TRGDC, comprising a VH having an amino acid sequence of SEQ ID NO:4553. In one aspect, provided herein is an antibody that binds TRGDC, comprising a VL having an amino acid sequence of SEQ ID NO:4554. In one aspect, provided herein is an antibody that binds TRGDC, comprising a VH having an amino acid sequence of SEQ ID NO:4553, and a VL having an amino acid sequence of SEQ ID NO:4554. In one aspect, provided herein is an antibody that binds TRGDC, comprising a heavy chain having an amino acid sequence of SEQ ID NO:4555. In one aspect, provided herein is an antibody that binds TRGDC, comprising a light chain having an amino acid sequence of SEQ ID NO:4556. In one aspect, provided herein is an antibody that binds TRGDC, comprising a heavy chain having an amino acid sequence of SEQ ID NO:4555, and a light chain having an amino acid sequence of SEQ ID NO:4556. In one aspect, provided herein is an antibody that binds TRGDC, comprising a VH having an amino acid sequence having at least 95% identity to an amino acid sequence of SEQ ID NO:4553. In one aspect, provided herein is an antibody that binds TRGDC, comprising a VL having an amino acid sequence having at least 95% identity to an amino acid sequence of SEQ ID NO:4554. In one aspect, provided herein is an antibody that binds TRGDC, comprising a VH having an amino acid sequence having at least 95% identity to an amino acid sequence of SEQ ID NO:4553, and a VL having an amino acid sequence having at least 95% identity to an amino acid sequence of SEQ ID NO:4554. In one aspect, provided herein is an antibody that binds TRGDC, comprising a heavy chain having an amino acid sequence having at least 95% identity to an amino acid sequence of SEQ ID NO:4555. In one aspect, provided herein is an antibody that binds TRGDC, comprising a light chain having an amino acid sequence having at least 95% identity to an amino acid sequence of SEQ ID NO:4556. In one aspect, provided herein is an antibody that binds TRGDC, comprising a heavy chain having an amino acid sequence having at least 95% identity to an amino acid sequence of SEQ ID NO:4555, and a light chain having an amino acid sequence having at least 95% identity to an amino acid sequence of SEQ ID NO:4556.

In one aspect, provided herein is an antibody that binds TRGDC, comprising: (i) a VH comprising a VH CDR1, a VH CDR2, and a VH CDR3 having an amino acid sequence of SEQ ID NOs:4557, 4558, and 4559, respectively, and (ii) a VL comprising a VL CDR1, VL CDR2, and VL CDR3 having an amino acid sequence of SEQ ID NOs:4560, 4561, and 4562, respectively. In one aspect, provided herein is an antibody that binds TRGDC, comprising: (i) a VH comprising a VH CDR1, a VH CDR2, and a VH CDR3 having an amino acid sequence of SEQ ID NOs:4563, 4564, and 4565, respectively, and (ii) a VL comprising a VL CDR1, VL CDR2, and VL CDR3 having an amino acid sequence of SEQ ID NOs:4566, 4567, and 4568, respectively. In one aspect, provided herein is an antibody that binds TRGDC, comprising: (i) a VH comprising a VH CDR1, a VH CDR2, and a VH CDR3 having an amino acid sequence of SEQ ID NOs:4569, 4570, and 4571, respectively, and (ii) a VL comprising a VL CDR1, VL CDR2, and VL CDR3 having an amino acid sequence of SEQ ID NOs:4572, 4573, and 4574, respectively. In one aspect, provided herein is an antibody that binds TRGDC, comprising: (i) a VH comprising a VH CDR1, a VH CDR2, and a VH CDR3 having an amino acid sequence of SEQ ID NOs:4575, 4576, and 4577, respectively, and (ii) a VL comprising a VL CDR1, VL CDR2, and VL CDR3 having an amino acid sequence of SEQ ID NOs:4578, 4579, and 4580, respectively. In one aspect, provided herein is an antibody that binds TRGDC, comprising: (i) a VH comprising a VH CDR1, a VH CDR2, and a VH CDR3 having an amino acid sequence of SEQ ID NOs:4581, 4582, and 4583, respectively, and (ii) a VL comprising a VL CDR1, VL CDR2, and VL CDR3 having an amino acid sequence of SEQ ID NOs:4584, 4585, and 4586, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4587; and (ii) a VL comprising a VL CDR1, a VL CDR2, and a VL CDR3 having an amino acid sequence of a VL CDR1, a VL CDR2, and a VL CDR3, respectively, of SEQ ID NO:4588. In one aspect, provided herein is an antibody that binds TRGDC, comprising a VH having an amino acid sequence of SEQ ID NO:4587. In one aspect, provided herein is an antibody that binds TRGDC, comprising a VL having an amino acid sequence of SEQ ID NO:4588. In one aspect, provided herein is an antibody that binds TRGDC, comprising a VH having an amino acid sequence of SEQ ID NO:4587, and a VL having an amino acid sequence of SEQ ID NO:4588. In one aspect, provided herein is an antibody that binds TRGDC, comprising a heavy chain having an amino acid sequence of SEQ ID NO:4589. In one aspect, provided herein is an antibody that binds TRGDC, comprising a light chain having an amino acid sequence of SEQ ID NO:4590. In one aspect, provided herein is an antibody that binds TRGDC, comprising a heavy chain having an amino acid sequence of SEQ ID NO:4589, and a light chain having an amino acid sequence of SEQ ID NO:4590. In one aspect, provided herein is an antibody that binds TRGDC, comprising a VH having an amino acid sequence having at least 95% identity to an amino acid sequence of SEQ ID NO:4587. In one aspect, provided herein is an antibody that binds TRGDC, comprising a VL having an amino acid sequence having at least 95% identity to an amino acid sequence of SEQ ID NO:4588. In one aspect, provided herein is an antibody that binds TRGDC, comprising a VH having an amino acid sequence having at least 95% identity to an amino acid sequence of SEQ ID NO:4587, and a VL having an amino acid sequence having at least 95% identity to an amino acid sequence of SEQ ID NO:4588. In one aspect, provided herein is an antibody that binds TRGDC, comprising a heavy chain having an amino acid sequence having at least 95% identity to an amino acid sequence of SEQ ID NO:4589. In one aspect, provided herein is an antibody that binds TRGDC, comprising a light chain having an amino acid sequence having at least 95% identity to an amino acid sequence of SEQ ID NO:4590. In one aspect, provided herein is an antibody that binds TRGDC, comprising a heavy chain having an amino acid sequence having at least 95% identity to an amino acid sequence of SEQ ID NO:4589, and a light chain having an amino acid sequence having at least 95% identity to an amino acid sequence of SEQ ID NO:4590.

In one aspect, provided herein is an antibody that binds TRGDC, comprising: (i) a VH comprising a VH CDR1, a VH CDR2, and a VH CDR3 having an amino acid sequence of SEQ ID NOs:4591, 4592, and 4593, respectively, and (ii) a VL comprising a VL CDR1, VL CDR2, and VL CDR3 having an amino acid sequence of SEQ ID NOs:4594, 4595, and 4596, respectively. In one aspect, provided herein is an antibody that binds TRGDC, comprising: (i) a VH comprising a VH CDR1, a VH CDR2, and a VH CDR3 having an amino acid sequence of SEQ ID NOs:4597, 4598, and 4599, respectively, and (ii) a VL comprising a VL CDR1, VL CDR2, and VL CDR3 having an amino acid sequence of SEQ ID NOs:4600, 4601, and 4602, respectively. In one aspect, provided herein is an antibody that binds TRGDC, comprising: (i) a VH comprising a VH CDR1, a VH CDR2, and a VH CDR3 having an amino acid sequence of SEQ ID NOs:4603, 4604, and 4605, respectively, and (ii) a VL comprising a VL CDR1, VL CDR2, and VL CDR3 having an amino acid sequence of SEQ ID NOs:4606, 4607, and 4608, respectively. In one aspect, provided herein is an antibody that binds TRGDC, comprising: (i) a VH comprising a VH CDR1, a VH CDR2, and a VH CDR3 having an amino acid sequence of SEQ ID NOs:4609, 4610, and 4611, respectively, and (ii) a VL comprising a VL CDR1, VL CDR2, and VL CDR3 having an amino acid sequence of SEQ ID NOs:4612, 4613, and 4614, respectively. In one aspect, provided herein is an antibody that binds TRGDC, comprising: (i) a VH comprising a VH CDR1, a VH CDR2, and a VH CDR3 having an amino acid sequence of SEQ ID NOs:4615, 4616, and 4617, respectively, and (ii) a VL comprising a VL CDR1, VL CDR2, and VL CDR3 having an amino acid sequence of SEQ ID NOs:4618, 4619, and 4620, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4621; and (ii) a VL comprising a VL CDR1, a VL CDR2, and a VL CDR3 having an amino acid sequence of a VL CDR1, a VL CDR2, and a VL CDR3, respectively, of SEQ ID NO:4622. In one aspect, provided herein is an antibody that binds TRGDC, comprising a VH having an amino acid sequence of SEQ ID NO:4621. In one aspect, provided herein is an antibody that binds TRGDC, comprising a VL having an amino acid sequence of SEQ ID NO:4622. In one aspect, provided herein is an antibody that binds TRGDC, comprising a VH having an amino acid sequence of SEQ ID NO:4621, and a VL having an amino acid sequence of SEQ ID NO:4622. In one aspect, provided herein is an antibody that binds TRGDC, comprising a heavy chain having an amino acid sequence of SEQ ID NO:4623. In one aspect, provided herein is an antibody that binds TRGDC, comprising a light chain having an amino acid sequence of SEQ ID NO:4624. In one aspect, provided herein is an antibody that binds TRGDC, comprising a heavy chain having an amino acid sequence of SEQ ID NO:4623, and a light chain having an amino acid sequence of SEQ ID NO:4624. In one aspect, provided herein is an antibody that binds TRGDC, comprising a VH having an amino acid sequence having at least 95% identity to an amino acid sequence of SEQ ID NO:4621. In one aspect, provided herein is an antibody that binds TRGDC, comprising a VL having an amino acid sequence having at least 95% identity to an amino acid sequence of SEQ ID NO:4622. In one aspect, provided herein is an antibody that binds TRGDC, comprising a VH having an amino acid sequence having at least 95% identity to an amino acid sequence of SEQ ID NO:4621, and a VL having an amino acid sequence having at least 95% identity to an amino acid sequence of SEQ ID NO:4622. In one aspect, provided herein is an antibody that binds TRGDC, comprising a heavy chain having an amino acid sequence having at least 95% identity to an amino acid sequence of SEQ ID NO:4623. In one aspect, provided herein is an antibody that binds TRGDC, comprising a light chain having an amino acid sequence having at least 95% identity to an amino acid sequence of SEQ ID NO:4624. In one aspect, provided herein is an antibody that binds TRGDC, comprising a heavy chain having an amino acid sequence having at least 95% identity to an amino acid sequence of SEQ ID NO:4623, and a light chain having an amino acid sequence having at least 95% identity to an amino acid sequence of SEQ ID NO:4624.

In one aspect, provided herein is an antibody that binds TRGDC, comprising: (i) a VH comprising a VH CDR1, a VH CDR2, and a VH CDR3 having an amino acid sequence of SEQ ID NOs:4625, 4626, and 4627, respectively, and (ii) a VL comprising a VL CDR1, VL CDR2, and VL CDR3 having an amino acid sequence of SEQ ID NOs:4628, 4629, and 4630, respectively. In one aspect, provided herein is an antibody that binds TRGDC, comprising: (i) a VH comprising a VH CDR1, a VH CDR2, and a VH CDR3 having an amino acid sequence of SEQ ID NOs:4631, 4632, and 4633, respectively, and (ii) a VL comprising a VL CDR1, VL CDR2, and VL CDR3 having an amino acid sequence of SEQ ID NOs:4634, 4635, and 4636, respectively. In one aspect, provided herein is an antibody that binds TRGDC, comprising: (i) a VH comprising a VH CDR1, a VH CDR2, and a VH CDR3 having an amino acid sequence of SEQ ID NOs:4637, 4638, and 4639, respectively, and (ii) a VL comprising a VL CDR1, VL CDR2, and VL CDR3 having an amino acid sequence of SEQ ID NOs:4640, 4641, and 4642, respectively. In one aspect, provided herein is an antibody that binds TRGDC, comprising: (i) a VH comprising a VH CDR1, a VH CDR2, and a VH CDR3 having an amino acid sequence of SEQ ID NOs:4643, 4644, and 4645, respectively, and (ii) a VL comprising a VL CDR1, VL CDR2, and VL CDR3 having an amino acid sequence of SEQ ID NOs:4646, 4647, and 4648, respectively. In one aspect, provided herein is an antibody that binds TRGDC, comprising: (i) a VH comprising a VH CDR1, a VH CDR2, and a VH CDR3 having an amino acid sequence of SEQ ID NOs:4649, 4650, and 4651, respectively, and (ii) a VL comprising a VL CDR1, VL CDR2, and VL CDR3 having an amino acid sequence of SEQ ID NOs:4652, 4653, and 4654, respectively. In one aspect, provided herein is an antibody that binds TRGDC, comprising: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4655; and (ii) a VL comprising a VL CDR1, a VL CDR2, and a VL CDR3 having an amino acid sequence of a VL CDR1, a VL CDR2, and a VL CDR3, respectively, of SEQ ID NO:4656. In one aspect, provided herein is an antibody that binds TRGDC, comprising a VH having an amino acid sequence of SEQ ID NO:4655. In one aspect, provided herein is an antibody that binds TRGDC, comprising a VL having an amino acid sequence of SEQ ID NO:4656. In one aspect, provided herein is an antibody that binds TRGDC, comprising a VH having an amino acid sequence of SEQ ID NO:4655, and a VL having an amino acid sequence of SEQ ID NO:4656. In one aspect, provided herein is an antibody that binds TRGDC, comprising a heavy chain having an amino acid sequence of SEQ ID NO:4657. In one aspect, provided herein is an antibody that binds TRGDC, comprising a light chain having an amino acid sequence of SEQ ID NO:4658. In one aspect, provided herein is an antibody that binds TRGDC, comprising a heavy chain having an amino acid sequence of SEQ ID NO:4657, and a light chain having an amino acid sequence of SEQ ID NO:4658. In one aspect, provided herein is an antibody that binds TRGDC, comprising a VH having an amino acid sequence having at least 95% identity to an amino acid sequence of SEQ ID NO:4655. In one aspect, provided herein is an antibody that binds TRGDC, comprising a VL having an amino acid sequence having at least 95% identity to an amino acid sequence of SEQ ID NO:4656. In one aspect, provided herein is an antibody that binds TRGDC, comprising a VH having an amino acid sequence having at least 95% identity to an amino acid sequence of SEQ ID NO:4655, and a VL having an amino acid sequence having at least 95% identity to an amino acid sequence of SEQ ID NO:4656. In one aspect, provided herein is an antibody that binds TRGDC, comprising a heavy chain having an amino acid sequence having at least 95% identity to an amino acid sequence of SEQ ID NO:4657. In one aspect, provided herein is an antibody that binds TRGDC, comprising a light chain having an amino acid sequence having at least 95% identity to an amino acid sequence of SEQ ID NO:4658. In one aspect, provided herein is an antibody that binds TRGDC, comprising a heavy chain having an amino acid sequence having at least 95% identity to an amino acid sequence of SEQ ID NO:4657, and a light chain having an amino acid sequence having at least 95% identity to an amino acid sequence of SEQ ID NO:4658.

In one aspect, provided herein is an antibody that binds TRGDC. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4145. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4146. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4145; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4146. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4179. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4180. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4179; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4180. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4213. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4214. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4213; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4214. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4247. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4248. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4247; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4248. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4281. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4282. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4281; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4282. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4315. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4316. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4315; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4316. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4349. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4350. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4349; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4350. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4383. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4384. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4383; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4384. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4417. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4418. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4417; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4418. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4451. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4452. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4451; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4452. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4485. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4486. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4485; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4486. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4519. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4520. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4519; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4520. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4553. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4554. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4553; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4554. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4587. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4588. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4587; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4588. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4621. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4622. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4621; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4622. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4655. In one embodiment, the TRGDC antibody comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4656. In one embodiment, the TRGDC antibody comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4655; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4656. In some embodiments, the TRGDC antibody is a multispecific antibody. In some embodiments, the TRGDC antibody is a bispecific antibody. In some embodiments, the TRGDC antibody is a trispecific antibody. In some embodiments, the TRGDC antibody is a quadraspecific antibody.

In one aspect, provided herein is an antibody that binds TRGDC, comprising a VH CDR1 having an amino acid sequence of SEQ ID NO:4139, wherein the eighth amino acid is R or S; a VH CDR2 having an amino acid sequence of SEQ ID NO:4140; a VH CDR3 having an amino acid sequence of SEQ ID NO:4141, wherein the third amino acid is D or G, the fourth amino acid is E or L, the sixth amino acid is E or G, the ninth amino acid is E or D, the eleventh amino acid is C or F. In one aspect, provided herein is an antibody that binds TRGDC, comprising a VL CDR1 having an amino acid sequence of SEQ ID NO:4142, wherein the fourth amino acid is H or S; a VL CDR2 having an amino acid sequence of SEQ ID NO:4143; and a VL CDR3 having an amino acid sequence of SEQ ID NO:4144, wherein the eighth amino acid is F or W. In one aspect, provided herein is an antibody that binds TRGDC, comprising a VH CDR1 having an amino acid sequence of SEQ ID NO:4139, wherein the eighth amino acid is R or S; a VH CDR2 having an amino acid sequence of SEQ ID NO:4140; a VH CDR3 having an amino acid sequence of SEQ ID NO:4141, wherein the third amino acid is D or G, the fourth amino acid is E or L, the sixth amino acid is E or G, the ninth amino acid is E or D, the eleventh amino acid is C or F; a VL CDR1 having an amino acid sequence of SEQ ID NO:4142, wherein the fourth amino acid is H or S; a VL CDR2 having an amino acid sequence of SEQ ID NO:4143; and a VL CDR3 having an amino acid sequence of SEQ ID NO:4144, wherein the eighth amino acid is F or W.

In one aspect, provided herein is an antibody that binds TRGDC, comprising a VH CDR1 having an amino acid sequence of SEQ ID NO:4173, wherein the first amino acid is G or R, the fourth amino acid is F or L, the sixth amino acid is N or S; a VH CDR2 having an amino acid sequence of SEQ ID NO:4174; a VH CDR3 having an amino acid sequence of SEQ ID NO:4175, wherein the fourth amino acid is R or V, the seventh amino acid is G or T, the eighth amino acid is G or T, there is no ninth amino acid or is T, the sixteenth amino acid is L or M. In one aspect, provided herein is an antibody that binds TRGDC, comprising a VL CDR1 having an amino acid sequence of SEQ ID NO:4176, wherein the fourth amino acid is A or S; a VL CDR2 having an amino acid sequence of SEQ ID NO:4177; and a VL CDR3 having an amino acid sequence of SEQ ID NO:4178. In one aspect, provided herein is an antibody that binds TRGDC, comprising a VH CDR1 having an amino acid sequence of SEQ ID NO:4173, wherein the first amino acid is G or R, the fourth amino acid is F or L, the sixth amino acid is N or S; a VH CDR2 having an amino acid sequence of SEQ ID NO:4174; a VH CDR3 having an amino acid sequence of SEQ ID NO:4175, wherein the fourth amino acid is R or V, the seventh amino acid is G or T, the eighth amino acid is G or T, there is no ninth amino acid or is T, the sixteenth amino acid is L or M; a VL CDR1 having an amino acid sequence of SEQ ID NO:4176, wherein the fourth amino acid is A or S; a VL CDR2 having an amino acid sequence of SEQ ID NO:4177; and a VL CDR3 having an amino acid sequence of SEQ ID NO:4178.

In one aspect, provided herein is an antibody that binds TRGDC, comprising a VH CDR1 having an amino acid sequence of SEQ ID NO:4479, wherein the third amino acid is S or T, the sixth amino acid is N or S; a VH CDR2 having an amino acid sequence of SEQ ID NO:4480, wherein the fourth amino acid is I or F, the sixth amino acid is G or S, the seventh amino acid is A or T, the eighth amino acid is A, G, or P; a VH CDR3 having an amino acid sequence of SEQ ID NO:4481, wherein the first amino acid is S or no amino acid in this position, the second amino acid is S, T, or no amino acid in this position, the third amino acid is A, G, or S, the fourth amino acid is G, N, or T, the fifth amino acid is A, G, or S, the sixth amino acid is G, T, or Y, the seventh amino acid is G, T, or V, the eighth amino acid is D, T, or Y, the ninth amino acid is T or Y, the fifteenth amino acid is I or M, the sixteenth amino acid is D or N. In one aspect, provided herein is an antibody that binds TRGDC, comprising a VL CDR1 having an amino acid sequence of SEQ ID NO:4482; a VL CDR2 having an amino acid sequence of SEQ ID NO:4483, wherein the first amino acid is E or K, the third amino acid is F or S; and a VL CDR3 having an amino acid sequence of SEQ ID NO:4484, wherein the fifth amino acid is E or Q. In one aspect, provided herein is an antibody that binds TRGDC, comprising a VH CDR1 having an amino acid sequence of SEQ ID NO:4479, wherein the third amino acid is S or T, the sixth amino acid is N or S; a VH CDR2 having an amino acid sequence of SEQ ID NO:4480, wherein the fourth amino acid is I or F, the sixth amino acid is G or S, the seventh amino acid is A or T, the eighth amino acid is A, G, or P; a VH CDR3 having an amino acid sequence of SEQ ID NO:4481, wherein the first amino acid is S or no amino acid in this position, the second amino acid is S, T, or no amino acid in this position, the third amino acid is A, G, or S, the fourth amino acid is G, N, or T, the fifth amino acid is A, G, or S, the sixth amino acid is G, T, or Y, the seventh amino acid is G, T, or V, the eighth amino acid is D, T, or Y, the ninth amino acid is T or Y, the fifteenth amino acid is I or M, the sixteenth amino acid is D or N; a VL CDR1 having an amino acid sequence of SEQ ID NO:4482; a VL CDR2 having an amino acid sequence of SEQ ID NO:4483, wherein the first amino acid is E or K, the third amino acid is F or S; and a VL CDR3 having an amino acid sequence of SEQ ID NO:4484, wherein the fifth amino acid is E or Q.

In one aspect, provided herein is an antibody that binds TRGDC, comprising a VH CDR1 having an amino acid sequence of SEQ ID NO:4241; a VH CDR2 having an amino acid sequence of SEQ ID NO:4242, wherein the seventh amino acid is A or Y; a VH CDR3 having an amino acid sequence of SEQ ID NO:4243, wherein the third amino acid is E or L, the ninth amino acid is I or V. In one aspect, provided herein is an antibody that binds TRGDC, comprising a VL CDR1 having an amino acid sequence of SEQ ID NO:4244, wherein the first amino acid is N or S, the fourth amino acid is N or S; a VL CDR2 having an amino acid sequence of SEQ ID NO:4245; and a VL CDR3 having an amino acid sequence of SEQ ID NO:4246, wherein the fifth amino acid is N or S, the tenth amino acid is V or W. In one aspect, provided herein is an antibody that binds TRGDC, comprising a VH CDR1 having an amino acid sequence of SEQ ID NO:4241; a VH CDR2 having an amino acid sequence of SEQ ID NO:4242, wherein the seventh amino acid is A or Y; a VH CDR3 having an amino acid sequence of SEQ ID NO:4243, wherein the third amino acid is E or L, the ninth amino acid is I or V; a VL CDR1 having an amino acid sequence of SEQ ID NO:4244, wherein the first amino acid is N or S, the fourth amino acid is N or S; a VL CDR2 having an amino acid sequence of SEQ ID NO:4245; and a VL CDR3 having an amino acid sequence of SEQ ID NO:4246, wherein the fifth amino acid is N or S, the tenth amino acid is V or W.

In another aspect, provided herein is a multispecific antibody that binds TRGV9. In some embodiments, the multispecific antibody is a bispecific antibody. In some embodiments, the multispecific antibody is a trispecific antibody. In some embodiments, the multispecific antibody is a quadraspecific antibody. In one embodiment, the multispecific TRGV9 antibody comprises: (a) a first binding domain that binds TRGV9, and (b) a second binding domain that binds to a second target. In one embodiment, the multispecific TRGV9 antibody comprises: (a) a first binding domain that binds TRGV9, and (b) a second binding domain that binds to a second target, and (c) a third binding domain that binds to a third target. In one embodiment, the multispecific TRGV9 antibody comprises: (a) a first binding domain that binds TRGV9, and (b) a second binding domain that binds to a second target, (c) a third binding domain that binds to a third target, and (d) a fourth binding domain that binds to a fourth target.

In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:168. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:168. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:201. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:202. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:201; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:202. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:269. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:269; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:304. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:304. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:337. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:338. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:337; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:338. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:440. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:440. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:609. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:610. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:609; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:610. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:644. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:644. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:712. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:712. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:847. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:848. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:847; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:848. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:881. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:882. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:881; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:882. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:915. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:916. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:915; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:916. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:949. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:950. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:949; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:950. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:983. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:984. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:983; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:984. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1017. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1018. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1017; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1018. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1051. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1052. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1051; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1052. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1085. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1086. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1085; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1086. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1119. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1120. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1119; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1120. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1153. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1154. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1153; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1154. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1187. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1188. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1187; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1188. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1221. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1222. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1221; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1222. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1255. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1256. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1255; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1256. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1289. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1290. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1289; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1290. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1323. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1324. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1323; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1324. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1357. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1358. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1357; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1358. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1391. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1392. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1391; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1392. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1425. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1426. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1425; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1426. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1459. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1460. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1459; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1460. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1493. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1494. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1493; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1494. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1527. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1528. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1527; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1528. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1561. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1562. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1561; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1562. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1595. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1596. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1595; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1596. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1629. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1630. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1629; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1630. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1663. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1664. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1663; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1664. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1697. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1698. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1697; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1698. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1731. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1732. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1731; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1732. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1765. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1766. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1765; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1766. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1799. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1800. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1799; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1800. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1833. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1834. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1833; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1834. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1867. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1868. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1867; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1868. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1901. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1902. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1901; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1902. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1935. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1936. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1935; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1936. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1969. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1970. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1969; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1970. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2003. In one embodiment, the first binding domain that binds TRGV9 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2004. In one embodiment, the first binding domain that binds TRGV9 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2003; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2004.

In another aspect, provided herein is a multispecific antibody that binds TRDV2. In some embodiments, the multispecific antibody is a bispecific antibody. In some embodiments, the multispecific antibody is a trispecific antibody. In some embodiments, the multispecific antibody is a quadraspecific antibody. In one embodiment, the multispecific TRDV2 antibody comprises: (a) a first binding domain that binds TRDV2, and (b) a second binding domain that binds to a second target. In one embodiment, the multispecific TRDV2 antibody comprises: (a) a first binding domain that binds TRDV2, and (b) a second binding domain that binds to a second target, and (c) a third binding domain that binds to a third target. In one embodiment, the multispecific TRDV2 antibody comprises: (a) a first binding domain that binds TRDV2, and (b) a second binding domain that binds to a second target, (c) a third binding domain that binds to a third target, and (d) a fourth binding domain that binds to a fourth target.

In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2037. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2038. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2037; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2038. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2071. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2072. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2071; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2072. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2105. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2106. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2105; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2106. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2139. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2140. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2139; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2140. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2173. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2174. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2173; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2174. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2207. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2208. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2207; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2208. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2241. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2242. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2241; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2242. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2275. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2276. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2275; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2276. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2309. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2310. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2309; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2310. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2343. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2344. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2343; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2344. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2377. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2378. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2377; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2378. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2411. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2412. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2411; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2412. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2445. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2446. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2445; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2446. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2479. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2480. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2479; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2480. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2513. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2514. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2513; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2514. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2547. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2548. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2547; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2548. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2581. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2582. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2581; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2582. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2615. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2616. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2615; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2616. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2649. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2650. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2649; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2650. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2683. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2684. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2683; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2684. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2717. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2718. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2717; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2718. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2751. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2752. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2751; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2752. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2785. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2786. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2785; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2786. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2819. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2820. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2819; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2820. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2853. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2854. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2853; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2854. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2887. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2888. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2887; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2888. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2921. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2922. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2921; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2922. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2955. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2956. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2955; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2956. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2989. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2990. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2989; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2990. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3023. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3024. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3023; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3024. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3057. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3058. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3057; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3058. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3091. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3092. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3091; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3092. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3125. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3126. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3125; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3126. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3159. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3160. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3159; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3160. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3193. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3194. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3193; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3194. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3227. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3228. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3227; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3228. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3261. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3262. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3261; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3262. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3295. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3296. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3295; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3296. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3329. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3330. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3329; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3330. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3363. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3364. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3363; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3364. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3397. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3398. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3397; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3398. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3431. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3432. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3431; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3432. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3465. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3466. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3465; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3466. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3499. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3500. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3499; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3500. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3533. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3534. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3533; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3534. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3567. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3568. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3567; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3568. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3601. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3602. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3601; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3602. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3635. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3636. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3635; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3636. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3669. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3670. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3669; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3670. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3703. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3704. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3703; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3704. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3737. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3738. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3737; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3738. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3771. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3772. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3771; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3772. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3805. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3806. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3805; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3806. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3839. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3840. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3839; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3840. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3873. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3874. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3873; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3874. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3907. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3908. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3907; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3908. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3941. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3942. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3941; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3942. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3975. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3976. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3975; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3976. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4009. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4010. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4009; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4010. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4043. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4044. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4043; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4044. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4077. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4078. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4077; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4078. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4111. In one embodiment, the first binding domain that binds TRDV2 comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4112. In one embodiment, the first binding domain that binds TRDV2 comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4111; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4112.

In another aspect, provided herein is a multispecific antibody that binds TRGDC. In some embodiments, the multispecific antibody is a bispecific antibody. In some embodiments, the multispecific antibody is a trispecific antibody. In some embodiments, the multispecific antibody is a quadraspecific antibody. In one embodiment, the multispecific TRGDC antibody comprises: (a) a first binding domain that binds TRGDC, and (b) a second binding domain that binds to a second target. In one embodiment, the multispecific TRGDC antibody comprises: (a) a first binding domain that binds TRGDC, and (b) a second binding domain that binds to a second target, and (c) a third binding domain that binds to a third target. In one embodiment, the multispecific TRGDC antibody comprises: (a) a first binding domain that binds TRGDC, and (b) a second binding domain that binds to a second target, (c) a third binding domain that binds to a third target, and (d) a fourth binding domain that binds to a fourth target.

In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4145. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4146. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4145; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4146. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4179. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4180. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4179; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4180. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4213. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4214. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4213; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4214. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4247. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4248. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4247; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4248. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4281. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4282. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4281; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4282. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4315. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4316. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4315; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4316. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4349. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4350. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4349; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4350. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4383. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4384. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4383; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4384. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4417. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4418. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4417; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4418. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4451. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4452. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4451; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4452. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4485. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4486. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4485; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4486. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4519. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4520. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4519; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4520. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4553. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4554. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4553; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4554. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4587. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4588. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4587; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4588. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4621. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4622. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4621; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4622. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4655. In one embodiment, the first binding domain that binds TRGDC comprises: a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4656. In one embodiment, the first binding domain that binds TRGDC comprises: a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4655; and a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4656.

In some embodiments, a multispecific antibody provided herein is a diabody, a cross-body, or a multispecific antibody obtained via a controlled Fab arm exchange as those described herein.

In some embodiments, the multispecific antibodies include IgG-like molecules with complementary CH3 domains that promote heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus) and the DuoBody (Genmab A/S).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (ImClone/Eli Lilly), Ts2Ab (Medlmmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)₂-Fab (National Research Center for Antibody Medicine—China).

In some embodiments, Fab fusion bispecific antibodies include F(ab)₂ (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Full length bispecific antibodies provided herein can be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent mono specific antibody molecule and simultaneously CH3 domains of the parent antibodies release and reform by dissociation-association. The CH3 domains of the Fab arms can be engineered to favor heterodimerization over homodimerization. The resulting product is a bispecific antibody having two Fab arms or half molecules which each binding a distinct epitope, e.g., an epitope on CD8 and an epitope on CD4. Other methods of making multispecific antibodies are known and contemplated.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Publ. No. WO2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T3661_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W as described in U.S. Pat. Publ. No. US2012/0149876 or U.S. Pat. Publ. No. US2013/0195849.

In addition to methods described above, bispecific antibodies provided herein can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO2011/131746. In the methods, the first monospecific bivalent antibody and the second monospecific bivalent antibody are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions can optionally be restored to non-reducing conditions. Exemplary reducing agents that can be used are 2-mercaptoethylamine (2-MEA), dithiothreitol (DTT), dithioerythritol (DTE), glutathione, tris (2-carboxyethyl) phosphine (TCEP), L-cysteine and beta-mercaptoethanol, preferably a reducing agent selected from the group consisting of: 2-mercaptoethylamine, dithiothreitol and tris (2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 can be used.

In some embodiments, the TRGV9 antibody comprises a single chain antibody. In some embodiments, the TRGV9 antibody comprises a single domain antibody. In certain embodiments, the TRGV9 antibody comprises a nanobody. In certain embodiments, the TRGV9 antibody comprises a VHH antibody. In certain embodiments, the TRGV9 antibody comprises a llama antibody. In some embodiments, the TRGV9 antibody does not comprise a single chain antibody. In some embodiments, the TRGV9 antibody does not comprise a single domain antibody. In certain embodiments, the TRGV9 antibody does not comprise a nanobody. In certain embodiments, the TRGV9 antibody does not comprise a VHH antibody. In certain embodiments, the TRGV9 antibody does not comprise a llama antibody.

In some embodiments, the TRGV9 multispecific antibody comprises a single chain antibody. In some embodiments, the TRGV9 multispecific antibody comprises a single domain antibody. In certain embodiments, the TRGV9 multispecific antibody comprises a nanobody. In certain embodiments, the TRGV9 multispecific antibody comprises a VHH antibody. In certain embodiments, the TRGV9 multispecific antibody comprises a llama antibody. In some embodiments, the TRGV9 multispecific antibody does not comprise a single chain antibody. In some embodiments, the TRGV9 multispecific antibody does not comprise a single domain antibody. In certain embodiments, the TRGV9 multispecific antibody does not comprise a nanobody. In certain embodiments, the TRGV9 multispecific antibody does not comprise a VHH antibody. In certain embodiments, the TRGV9 multispecific antibody does not comprise a llama antibody.

According to another particular aspect, provided herein is a TRGV9 antibody or antigen-binding fragment thereof that induces antibody-dependent cell-mediated cytotoxicity (ADCC). The antibody or antigen-binding fragment thereof can, for example, induce ADCC in vitro.

In certain embodiments, the antibody or antigen-binding fragment thereof induces T cell dependent cytotoxicity of a second cell in vitro with an EC₅₀ of less than about 160 pM, when assessed in vitro at an effector to target cell ratio of 1:1.

In some embodiments, TRGV9 is present on the surface of a T cell. In some embodiments, the TRGV9 is present on the surface of a T cell, and the second target antigen is on the surface of a second cell. In some embodiments, the second cell is killed when the multispecific antibody binds to the TRGV9 on the surface of the T cell and the second target antigen on the surface of the second cell.

In some embodiments, the TRDV2 antibody comprises a single chain antibody. In some embodiments, the TRDV2 antibody comprises a single domain antibody. In certain embodiments, the TRDV2 antibody comprises a nanobody. In certain embodiments, the TRDV2 antibody comprises a VHH antibody. In certain embodiments, the TRDV2 antibody comprises a llama antibody. In some embodiments, the TRDV2 antibody does not comprise a single chain antibody. In some embodiments, the TRDV2 antibody does not comprise a single domain antibody. In certain embodiments, the TRDV2 antibody does not comprise a nanobody. In certain embodiments, the TRDV2 antibody does not comprise a VHH antibody. In certain embodiments, the TRDV2 antibody does not comprise a llama antibody.

In some embodiments, the TRDV2 multispecific antibody comprises a single chain antibody. In some embodiments, the TRDV2 multispecific antibody comprises a single domain antibody. In certain embodiments, the TRDV2 multispecific antibody comprises a nanobody. In certain embodiments, the TRDV2 multispecific antibody comprises a VHH antibody. In certain embodiments, the TRDV2 multispecific antibody comprises a llama antibody. In some embodiments, the TRDV2 multispecific antibody does not comprise a single chain antibody. In some embodiments, the TRDV2 multispecific antibody does not comprise a single domain antibody. In certain embodiments, the TRDV2 multispecific antibody does not comprise a nanobody. In certain embodiments, the TRDV2 multispecific antibody does not comprise a VHH antibody. In certain embodiments, the TRDV2 multispecific antibody does not comprise a llama antibody.

According to another particular aspect, provided herein is a TRDV2 antibody or antigen-binding fragment thereof that induces antibody-dependent cell-mediated cytotoxicity (ADCC). The antibody or antigen-binding fragment thereof can, for example, induce ADCC in vitro.

In certain embodiments, the antibody or antigen-binding fragment thereof induces T cell dependent cytotoxicity of a second cell in vitro with an EC₅₀ of less than about 160 pM, when assessed in vitro at an effector to target cell ratio of 1:1.

In some embodiments, TRDV2 is present on the surface of a T cell. In some embodiments, the TRDV2 is present on the surface of a T cell, and the second target antigen is on the surface of a second cell. In some embodiments, the second cell is killed when the multispecific antibody binds to the TRDV2 on the surface of the T cell and the second target antigen on the surface of the second cell.

In some embodiments, the TRGDC antibody comprises a single chain antibody. In some embodiments, the TRGDC antibody comprises a single domain antibody. In certain embodiments, the TRGDC antibody comprises a nanobody. In certain embodiments, the TRGDC antibody comprises a VHH antibody. In certain embodiments, the TRGDC antibody comprises a llama antibody. In some embodiments, the TRGDC antibody does not comprise a single chain antibody. In some embodiments, the TRGDC antibody does not comprise a single domain antibody. In certain embodiments, the TRGDC antibody does not comprise a nanobody. In certain embodiments, the TRGDC antibody does not comprise a VHH antibody. In certain embodiments, the TRGDC antibody does not comprise a llama antibody.

In some embodiments, the TRGDC multispecific antibody comprises a single chain antibody. In some embodiments, the TRGDC multispecific antibody comprises a single domain antibody. In certain embodiments, the TRGDC multispecific antibody comprises a nanobody. In certain embodiments, the TRGDC multispecific antibody comprises a VHH antibody. In certain embodiments, the TRGDC multispecific antibody comprises a llama antibody. In some embodiments, the TRGDC multispecific antibody does not comprise a single chain antibody. In some embodiments, the TRGDC multispecific antibody does not comprise a single domain antibody. In certain embodiments, the TRGDC multispecific antibody does not comprise a nanobody. In certain embodiments, the TRGDC multispecific antibody does not comprise a VHH antibody. In certain embodiments, the TRGDC multispecific antibody does not comprise a llama antibody.

According to another particular aspect, provided herein is a TRGDC antibody or antigen-binding fragment thereof that induces antibody-dependent cell-mediated cytotoxicity (ADCC). The antibody or antigen-binding fragment thereof can, for example, induce ADCC in vitro.

In certain embodiments, the antibody or antigen-binding fragment thereof induces T cell dependent cytotoxicity of a second cell in vitro with an EC₅₀ of less than about 160 pM, when assessed in vitro at an effector to target cell ratio of 1:1.

In some embodiments, TRGDC is present on the surface of a T cell. In some embodiments, the TRGDC is present on the surface of a T cell, and the second target antigen is on the surface of a second cell. In some embodiments, the second cell is killed when the multispecific antibody binds to the TRGDC on the surface of the T cell and the second target antigen on the surface of the second cell.

In some embodiments, the multispecific antibody induces T cell dependent cytotoxicity of the second cell in vitro with an EC₅₀ of less than about 500 pM. In some embodiments, the multispecific antibody induces T cell dependent cytotoxicity of the second cell in vitro with an EC₅₀ of less than about 300 pM. In some embodiments, the multispecific antibody induces γδ T cell dependent cytotoxicity of the second cell in vitro with an EC₅₀ of less than about 160 pM. In some embodiments, the EC₅₀ is assessed with a mixture of γδ T effector cells and target cells expressing the second target antigen. In some embodiments, the effector cell to target cell ratio is about 0.01 to 1 to about 5 to 1. In some embodiments, the effector cell to target cell ratio is about 0.1 to 1 to about 2 to 1. In some embodiments, the effector cell to target cell ratio is about 1:1.

In certain embodiments, the EC₅₀ is less than about 1000 pM, less than about 900 pM, less than about 800 pM, less than about 700 pM, less than about 600 pM, less than about 500 pM, less than about 400 pM, less than about 300 pM, less than about 200 pM, less than about 190 pM, less than about 180 pM, less than about 170 pM, less than about 160 pM, less than about 150 pM, less than about 140 pM, less than about 130 pM, less than about 120 pM, less than about 110 pM, less than about 100 pM, less than about 90 pM, less than about 80 pM, less than about 70 pM, less than about 60 pM, less than about 50 pM, less than about 40 pM, less than about 30 pM, less than about 20 pM, or less than about 10 pM.

In certain embodiments, the effector to target cell ratio can, for example, be 0.01:1, 0.02:1, 0.03:1, 0.04:1, 0.05:1, 0.06:1, 0.07:1, 0.08:1, 0.09:1, 1:1, 2:1, 3:1, 4:1, 5:1, 6:1, 7:1, 8:1, 9:1, or 10:1. In certain embodiments, the effector to target cell ratio can, for example, be between 0.01:1 and 10:1. In certain embodiments, the concentration of the multispecific antibody or antigen-binding fragment thereof is about 0.000005 ng/mL, about 0.00005 ng/mL, about 0.0005, about 0.005 ng/mL, about 0.01 ng/mL, about 0.02 ng/mL, about 0.03 ng/mL, about 0.04 ng/mL, about 0.05 ng/mL, about 0.06 ng/mL, about 0.07 ng/mL, about 0.08 ng/mL, about 0.09 ng/mL, about 0.1 ng/mL, about 0.5 ng/mL, about 1.0 ng/mL, about 10 ng/mL, about 20 ng/mL about, about 30 ng/mL about 40 ng/mL, about 50 ng/mL, about 60 ng/mL, about 70 ng/mL, about 80 ng/mL, about 90 ng/mL, about 100 ng/mL, or about 1000 ng/mL. In certain embodiments, the concentration of the multispecific antibody or antigen-binding fragment thereof is between about 0.000005 ng/mL and about 1000 ng/mL.

In another aspect, provided herein is an antibody that competes for binding to TRGV9 with any of the TRGV9 antibodies described herein. In another aspect, provided herein is an antibody that binds to the same epitope as any of the TRGV9 antibodies described herein. In another aspect, provided is a TRGV9 antibody that binds an epitope on TRGV9 that overlaps with the epitope on TRGV9 bound by a TRGV9 antibody described herein. In some embodiments, the TRGV9 antibody comprises a VH CDR1, VH CDR2, and VH CDR3 of a TRGV9 antibody provided herein. In some embodiments, the TRGV9 antibody comprises a VL CDR1, VL CDR2, and VL CDR3 of a TRGV9 antibody provided herein. In some embodiments, the TRGV9 antibody comprises a VH CDR1, VH CDR2, VH CDR3, a VL CDR1, VL CDR2, and VL CDR3 of a TRGV9 antibody provided herein. In some embodiments, the TRGV9 antibody comprises a VH of a TRGV9 antibody provided herein. In some embodiments, the TRGV9 antibody comprises a VL of a TRGV9 antibody provided herein. In some embodiments, the TRGV9 antibody comprises a VH and a VL of a TRGV9 antibody provided herein. In some embodiments, the TRGV9 antibody comprises a VH CDR1, VH CDR2, VH CDR3, a VL CDR1, VL CDR2, and VL CDR3 of a TRGV9 antibody provided herein. In some embodiments, the VH CDR1, VH CDR2, VH CDR3, VL CDR1, VL CDR2, and VL CDR3 amino acid sequences of the TRGV9 antibody are according to the Kabat numbering system. In some embodiments, the VH CDR1, VH CDR2, VH CDR3, VL CDR1, VL CDR2, and VL CDR3 amino acid sequences of the TRGV9 antibody are according to the Chothia numbering system. In some embodiments, the VH CDR1, VH CDR2, VH CDR3, VL CDR1, VL CDR2, and VL CDR3 amino acid sequences of the TRGV9 antibody are according to the AbM numbering system. In some embodiments, the VH CDR1, VH CDR2, VH CDR3, VL CDR1, VL CDR2, and VL CDR3 amino acid sequences of the TRGV9 antibody are according to the Contact numbering system. In some embodiments, the VH CDR1, VH CDR2, VH CDR3, VL CDR1, VL CDR2, and VL CDR3 amino acid sequences of the TRGV9 antibody are according to the IMGT numbering system. In certain embodiments, the TRGV9 antibody is a multispecific antibody. In some embodiments, the TRGV9 antibody is a bispecific antibody.

In another aspect, provided is an antibody that competes for binding to TRGV9 with a TRGV9 reference antibody. In another aspect, provided is a TRGV9 antibody that binds to the same TRGV9 epitope as a TRGV9 reference antibody. In another aspect, provided is a TRGV9 antibody that binds an epitope on TRGV9 that overlaps with the epitope on TRGV9 bound by a TRGV9 reference antibody. In some embodiments, the TRGV9 reference antibody comprises a VH CDR1, VH CDR2, and VH CDR3 of a TRGV9 reference antibody provided herein. In some embodiments, the TRGV9 reference antibody comprises a VL CDR1, VL CDR2, and VL CDR3 of a TRGV9 reference antibody provided herein. In some embodiments, the TRGV9 reference antibody comprises a VH CDR1, VH CDR2, VH CDR3, a VL CDR1, VL CDR2, and VL CDR3 of a TRGV9 reference antibody provided herein. In some embodiments, the TRGV9 reference antibody comprises a VH of a TRGV9 reference antibody provided herein. In some embodiments, the TRGV9 reference antibody comprises a VL of a TRGV9 reference antibody provided herein. In some embodiments, the TRGV9 reference antibody comprises a VH and a VL of a TRGV9 reference antibody provided herein. In some embodiments, the TRGV9 reference antibody comprises a VH CDR1, VH CDR2, VH CDR3, a VL CDR1, VL CDR2, and VL CDR3 of a TRGV9 reference antibody provided herein. In some embodiments, the VH CDR1, VH CDR2, VH CDR3, VL CDR1, VL CDR2, and VL CDR3 amino acid sequences of the TRGV9 reference antibody are according to the Kabat numbering system. In some embodiments, the VH CDR1, VH CDR2, VH CDR3, VL CDR1, VL CDR2, and VL CDR3 amino acid sequences of the TRGV9 reference antibody are according to the Chothia numbering system. In some embodiments, the VH CDR1, VH CDR2, VH CDR3, VL CDR1, VL CDR2, and VL CDR3 amino acid sequences of the TRGV9 reference antibody are according to the AbM numbering system. In some embodiments, the VH CDR1, VH CDR2, VH CDR3, VL CDR1, VL CDR2, and VL CDR3 amino acid sequences of the TRGV9 reference antibody are according to the Contact numbering system. In some embodiments, the VH CDR1, VH CDR2, VH CDR3, VL CDR1, VL CDR2, and VL CDR3 amino acid sequences of the TRGV9 reference antibody are according to the IMGT numbering system. In certain embodiments, the antibody is a multispecific antibody. In some embodiments, the antibody is a bispecific antibody. In certain embodiments, the TRGV9 reference antibody is a multispecific antibody. In some embodiments, the TRGV9 reference antibody is a bispecific antibody.

In another aspect, provided herein is an antibody that competes for binding to TRDV2 with any of the TRDV2 antibodies described herein. In another aspect, provided herein is an antibody that binds to the same epitope as any of the TRDV2 antibodies described herein. In another aspect, provided is a TRDV2 antibody that binds an epitope on TRDV2 that overlaps with the epitope on TRDV2 bound by a TRDV2 antibody described herein. In some embodiments, the TRDV2 antibody comprises a VH CDR1, VH CDR2, and VH CDR3 of a TRDV2 antibody provided herein. In some embodiments, the TRDV2 antibody comprises a VL CDR1, VL CDR2, and VL CDR3 of a TRDV2 antibody provided herein. In some embodiments, the TRDV2 antibody comprises a VH CDR1, VH CDR2, VH CDR3, a VL CDR1, VL CDR2, and VL CDR3 of a TRDV2 antibody provided herein. In some embodiments, the TRDV2 antibody comprises a VH of a TRDV2 antibody provided herein. In some embodiments, the TRDV2 antibody comprises a VL of a TRDV2 antibody provided herein. In some embodiments, the TRDV2 antibody comprises a VH and a VL of a TRDV2 antibody provided herein. In some embodiments, the TRDV2 antibody comprises a VH CDR1, VH CDR2, VH CDR3, a VL CDR1, VL CDR2, and VL CDR3 of a TRDV2 antibody provided herein. In some embodiments, the VH CDR1, VH CDR2, VH CDR3, VL CDR1, VL CDR2, and VL CDR3 amino acid sequences of the TRDV2 antibody are according to the Kabat numbering system. In some embodiments, the VH CDR1, VH CDR2, VH CDR3, VL CDR1, VL CDR2, and VL CDR3 amino acid sequences of the TRDV2 antibody are according to the Chothia numbering system. In some embodiments, the VH CDR1, VH CDR2, VH CDR3, VL CDR1, VL CDR2, and VL CDR3 amino acid sequences of the TRDV2 antibody are according to the AbM numbering system. In some embodiments, the VH CDR1, VH CDR2, VH CDR3, VL CDR1, VL CDR2, and VL CDR3 amino acid sequences of the TRDV2 antibody are according to the Contact numbering system. In some embodiments, the VH CDR1, VH CDR2, VH CDR3, VL CDR1, VL CDR2, and VL CDR3 amino acid sequences of the TRDV2 antibody are according to the IMGT numbering system. In certain embodiments, the TRDV2 antibody is a multispecific antibody. In some embodiments, the TRDV2 antibody is a bispecific antibody.

In another aspect, provided is an antibody that competes for binding to TRDV2 with a TRDV2 reference antibody. In another aspect, provided is a TRDV2 antibody that binds to the same TRDV2 epitope as a TRDV2 reference antibody. In another aspect, provided is a TRDV2 antibody that binds an epitope on TRDV2 that overlaps with the epitope on TRDV2 bound by a TRDV2 reference antibody. In some embodiments, the TRDV2 reference antibody comprises a VH CDR1, VH CDR2, and VH CDR3 of a TRDV2 reference antibody provided herein. In some embodiments, the TRDV2 reference antibody comprises a VL CDR1, VL CDR2, and VL CDR3 of a TRDV2 reference antibody provided herein. In some embodiments, the TRDV2 reference antibody comprises a VH CDR1, VH CDR2, VH CDR3, a VL CDR1, VL CDR2, and VL CDR3 of a TRDV2 reference antibody provided herein. In some embodiments, the TRDV2 reference antibody comprises a VH of a TRDV2 reference antibody provided herein. In some embodiments, the TRDV2 reference antibody comprises a VL of a TRDV2 reference antibody provided herein. In some embodiments, the TRDV2 reference antibody comprises a VH and a VL of a TRDV2 reference antibody provided herein. In some embodiments, the TRDV2 reference antibody comprises a VH CDR1, VH CDR2, VH CDR3, a VL CDR1, VL CDR2, and VL CDR3 of a TRDV2 reference antibody provided herein. In some embodiments, the VH CDR1, VH CDR2, VH CDR3, VL CDR1, VL CDR2, and VL CDR3 amino acid sequences of the TRDV2 reference antibody are according to the Kabat numbering system. In some embodiments, the VH CDR1, VH CDR2, VH CDR3, VL CDR1, VL CDR2, and VL CDR3 amino acid sequences of the TRDV2 reference antibody are according to the Chothia numbering system. In some embodiments, the VH CDR1, VH CDR2, VH CDR3, VL CDR1, VL CDR2, and VL CDR3 amino acid sequences of the TRDV2 reference antibody are according to the AbM numbering system. In some embodiments, the VH CDR1, VH CDR2, VH CDR3, VL CDR1, VL CDR2, and VL CDR3 amino acid sequences of the TRDV2 reference antibody are according to the Contact numbering system. In some embodiments, the VH CDR1, VH CDR2, VH CDR3, VL CDR1, VL CDR2, and VL CDR3 amino acid sequences of the TRDV2 reference antibody are according to the IMGT numbering system. In certain embodiments, the antibody is a multispecific antibody. In some embodiments, the antibody is a bispecific antibody. In certain embodiments, the TRDV2 reference antibody is a multispecific antibody. In some embodiments, the TRDV2 reference antibody is a bispecific antibody.

In another aspect, provided herein is an antibody that competes for binding to TRGDC with any of the TRGDC antibodies described herein. In another aspect, provided herein is an antibody that binds to the same epitope as any of the TRGDC antibodies described herein. In another aspect, provided is a TRGDC antibody that binds an epitope on TRGDC that overlaps with the epitope on TRGDC bound by a TRGDC antibody described herein. In some embodiments, the TRGDC antibody comprises a VH CDR1, VH CDR2, and VH CDR3 of a TRGDC antibody provided herein. In some embodiments, the TRGDC antibody comprises a VL CDR1, VL CDR2, and VL CDR3 of a TRGDC antibody provided herein. In some embodiments, the TRGDC antibody comprises a VH CDR1, VH CDR2, VH CDR3, a VL CDR1, VL CDR2, and VL CDR3 of a TRGDC antibody provided herein. In some embodiments, the TRGDC antibody comprises a VH of a TRGDC antibody provided herein. In some embodiments, the TRGDC antibody comprises a VL of a TRGDC antibody provided herein. In some embodiments, the TRGDC antibody comprises a VH and a VL of a TRGDC antibody provided herein. In some embodiments, the TRGDC antibody comprises a VH CDR1, VH CDR2, VH CDR3, a VL CDR1, VL CDR2, and VL CDR3 of a TRGDC 30 antibody provided herein. In some embodiments, the VH CDR1, VH CDR2, VH CDR3, VL CDR1, VL CDR2, and VL CDR3 amino acid sequences of the TRGDC antibody are according to the Kabat numbering system. In some embodiments, the VH CDR1, VH CDR2, VH CDR3, VL CDR1, VL CDR2, and VL CDR3 amino acid sequences of the TRGDC antibody are according to the Chothia numbering system. In some embodiments, the VH CDR1, VH CDR2, VH CDR3, VL CDR1, VL CDR2, and VL CDR3 amino acid sequences of the TRGDC antibody are according to the AbM numbering system. In some embodiments, the VH CDR1, VH CDR2, VH CDR3, VL CDR1, VL CDR2, and VL CDR3 amino acid sequences of the TRGDC antibody are according to the Contact numbering system. In some embodiments, the VH CDR1, VH CDR2, VH CDR3, VL CDR1, VL CDR2, and VL CDR3 amino acid sequences of the TRGDC antibody are according to the IMGT numbering system. In certain embodiments, the TRGDC antibody is a multispecific antibody. In some embodiments, the TRGDC antibody is a bispecific antibody.

In another aspect, provided is an antibody that competes for binding to TRGDC with a TRGDC reference antibody. In another aspect, provided is a TRGDC antibody that binds to the same TRGDC epitope as a TRGDC reference antibody. In another aspect, provided is a TRGDC antibody that binds an epitope on TRGDC that overlaps with the epitope on TRGDC bound by a TRGDC reference antibody. In some embodiments, the TRGDC reference antibody comprises a VH CDR1, VH CDR2, and VH CDR3 of a TRGDC reference antibody provided herein. In some embodiments, the TRGDC reference antibody comprises a VL CDR1, VL CDR2, and VL CDR3 of a TRGDC reference antibody provided herein. In some embodiments, the TRGDC reference antibody comprises a VH CDR1, VH CDR2, VH CDR3, a VL CDR1, VL CDR2, and VL CDR3 of a TRGDC reference antibody provided herein. In some embodiments, the TRGDC reference antibody comprises a VH of a TRGDC reference antibody provided herein. In some embodiments, the TRGDC reference antibody comprises a VL of a TRGDC reference antibody provided herein. In some embodiments, the TRGDC reference antibody comprises a VH and a VL of a TRGDC reference antibody provided herein. In some embodiments, the TRGDC reference antibody comprises a VH CDR1, VH CDR2, VH CDR3, a VL CDR1, VL CDR2, and VL CDR3 of a TRGDC reference antibody provided herein. In some embodiments, the VH CDR1, VH CDR2, VH CDR3, VL CDR1, VL CDR2, and VL CDR3 amino acid sequences of the TRGDC reference antibody are according to the Kabat numbering system. In some embodiments, the VH CDR1, VH CDR2, VH CDR3, VL CDR1, VL CDR2, and VL CDR3 amino acid sequences of the TRGDC reference antibody are according to the Chothia numbering system. In some embodiments, the VH CDR1, VH CDR2, VH CDR3, VL CDR1, VL CDR2, and VL CDR3 amino acid sequences of the TRGDC reference antibody are according to the AbM numbering system. In some embodiments, the VH CDR1, VH CDR2, VH CDR3, VL CDR1, VL CDR2, and VL CDR3 amino acid sequences of the TRGDC reference antibody are according to the Contact numbering system. In some embodiments, the VH CDR1, VH CDR2, VH CDR3, VL CDR1, VL CDR2, and VL CDR3 amino acid sequences of the TRGDC reference antibody are according to the IMGT numbering system. In certain embodiments, the antibody is a multispecific antibody. In some embodiments, the antibody is a bispecific antibody. In certain embodiments, the TRGDC reference antibody is a multispecific antibody. In some embodiments, the TRGDC reference antibody is a bispecific antibody.

In some embodiments described herein, immune effector properties of the antibodies provided herein can be enhanced or silenced through Fc modifications by techniques known to those skilled in the art. For example, Fc effector functions such as Clq binding, complement dependent cytotoxicity (CDC), antibody-dependent cell-mediated cytotoxicity (ADCC), antibody-dependent cell-mediated phagocytosis (ADCP), down regulation of cell surface receptors (e.g., B cell receptor; BCR), etc. can be provided and/or controlled by modifying residues in the Fc responsible for these activities.

“Antibody-dependent cell-mediated cytotoxicity” or “ADCC” refers to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell.

The ability of antibodies to induce ADCC can be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen binding or CDC activity. Such Abs can be achieved using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the α-1,6-fucosyltrasferase (FUT8) gene (Mori et al., Biotechnol Bioeng 88:901-908, 2004), or coexpression of β-1,4-N-acetylglucosaminyltransferase III and golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments described herein, ADCC elicited by the antibodies provided herein can also be enhanced by certain substitutions in the antibody Fc. Exemplary substitutions are for example substitutions at amino acid positions 256, 290, 298, 312, 356, 330, 333, 334, 360, 378 or 430 (residue numbering according to the EU index) as described in U.S. Pat. No. 6,737,056.

In some embodiments, a TRGV9 antibody provided herein is chimeric. In some embodiments, a TRGV9 antibody provided herein is human. In some embodiments, a TRGV9 antibody provided herein is humanized. In certain embodiments, a TRGV9 antibody provided herein is an isolated TRGV9 antibody. In some embodiments, a TRGV9 antigen binding fragment provided herein is chimeric. In some embodiments, a TRGV9 antigen binding fragment provided herein is human. In some embodiments, a TRGV9 antigen binding fragment provided herein is humanized. In certain embodiments, a TRGV9 antigen binding fragment provided herein is an isolated TRGV9 antigen binding fragment. In some embodiments, a TRGV9 antibody provided herein is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody. In some embodiments, a TRGV9 antibody provided herein is multivalent. In some embodiments, the antibody is capable of binding at least three antigens. In some embodiments, a TRGV9 antibody provided herein is multivalent. In some embodiments, the antibody is capable of binding at least four antigens. In some embodiments, the antibody is capable of binding at least five antigens.

In some embodiments, a TRGV9 multispecific antibody provided herein is chimeric. In some embodiments, a TRGV9 multispecific antibody provided herein is human. In some embodiments, a TRGV9 multispecific antibody provided herein is humanized. In certain embodiments, a TRGV9 multispecific antibody provided herein is an isolated TRGV9 multispecific antibody. In some embodiments, a TRGV9 multispecific antibody comprising a TRGV9 antigen binding fragment provided herein is chimeric. In some embodiments, a TRGV9 multispecific antibody comprising a TRGV9 antigen binding fragment provided herein is human. In some embodiments, a TRGV9 multispecific antibody comprising a TRGV9 antigen binding fragment provided herein is humanized. In certain embodiments, a TRGV9 multispecific antibody comprising a TRGV9 antigen binding fragment provided herein is an isolated TRGV9 multispecific antibody.

In some embodiments of the TRGV9 multispecific antibodies provided herein, the first binding domain is human. In some embodiments, the second binding domain is human. In some embodiments of the TRGV9 multispecific antibodies provided herein, both the first binding domain and the second binding domain are human. In some embodiments of the TRGV9 multispecific antibodies provided herein, the first binding domain is humanized. In some embodiments of the TRGV9 multispecific antibodies provided herein, the second binding domain is humanized. In some embodiments of the TRGV9 multispecific antibodies provided herein, both the first binding domain and the second binding domain are humanized. In some embodiments of the TRGV9 multispecific antibodies provided herein, both the first binding domain is human and the second binding domain is humanized. In some embodiments of the TRGV9 multispecific antibodies provided herein, both the first binding domain is humanized and the second binding domain is human.

In some embodiments, a TRGV9 multispecific antibody provided herein is multivalent. In some embodiments, the multispecific antibody is capable of binding at least three antigens. In some embodiments, the multispecific antibody is capable of binding at least five antigens. In certain embodiments, the multispecific antibody is a multispecific antibody. In some embodiments, a TRGV9 multispecific antibody provided herein is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody.

In certain embodiments, the antibodies provided herein are part of a multispecific antibody. In some embodiments, the multispecific antibody comprises a first binding domain that binds to a TRGV9 antigen. In some embodiments, the multispecific antibody comprises a first binding domain that binds to a TRGV9 antigen and comprises a second binding domain that binds to a second target antigen, as provided herein. In certain embodiments, the multispecific antibody binds to a TRGV9 antigen, a second target antigen, and one or more additional antigens. In some embodiments of the various antibodies provided herein, the antibody binds to an epitope of a given antigen.

In some embodiments, a TRDV2 antibody provided herein is chimeric. In some embodiments, a TRDV2 antibody provided herein is human. In some embodiments, a TRDV2 antibody provided herein is humanized. In certain embodiments, a TRDV2 antibody provided herein is an isolated TRDV2 antibody. In some embodiments, a TRDV2 antigen binding fragment provided herein is chimeric. In some embodiments, a TRDV2 antigen binding fragment provided herein is human. In some embodiments, a TRDV2 antigen binding fragment provided herein is humanized. In certain embodiments, a TRDV2 antigen binding fragment provided herein is an isolated TRDV2 antigen binding fragment. In some embodiments, a TRDV2 antibody provided herein is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody. In some embodiments, a TRDV2 antibody provided herein is multivalent. In some embodiments, the antibody is capable of binding at least three antigens. In some embodiments, the antibody is capable of binding at least four antigens. In some embodiments, the antibody is capable of binding at least five antigens.

In some embodiments, a TRDV2 multispecific antibody provided herein is 15 chimeric. In some embodiments, a TRDV2 multispecific antibody provided herein is human. In some embodiments, a TRDV2 multispecific antibody provided herein is humanized. In certain embodiments, a TRDV2 multispecific antibody provided herein is an isolated TRDV2 multispecific antibody. In some embodiments, a TRDV2 multispecific antibody comprising a TRDV2 antigen binding fragment provided herein is chimeric. In some embodiments, a TRDV2 multispecific antibody comprising a TRDV2 antigen binding fragment provided herein is human. In some embodiments, a TRDV2 multispecific antibody comprising a TRDV2 antigen binding fragment provided herein is humanized. In certain embodiments, a TRDV2 multispecific antibody comprising a TRDV2 antigen binding fragment provided herein is an isolated TRDV2 multispecific antibody.

In some embodiments of the TRDV2 multispecific antibodies provided herein, the first binding domain is human. In some embodiments, the second binding domain is human. In some embodiments of the TRDV2 multispecific antibodies provided herein, both the first binding domain and the second binding domain are human. In some embodiments of the TRDV2 multispecific antibodies provided herein, the first binding domain is humanized. In some embodiments of the TRDV2 multispecific antibodies provided herein, the second binding domain is humanized. In some embodiments of the TRDV2 multispecific antibodies provided herein, both the first binding domain and the second binding domain are humanized. In some embodiments of the TRDV2 multispecific antibodies provided herein, both the first binding domain is human and the second binding domain is humanized. In some embodiments of the TRDV2 multispecific antibodies provided herein, both the first binding domain is humanized and the second binding domain is human.

In some embodiments, a TRDV2 multispecific antibody provided herein is multivalent. In some embodiments, the multispecific antibody is capable of binding at least three antigens. In some embodiments, the multispecific antibody is capable of binding at least five antigens. In certain embodiments, the multispecific antibody is a multispecific antibody. In some embodiments, a TRDV2 multispecific antibody provided herein is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody.

In certain embodiments, the antibodies provided herein are part of a multispecific antibody. In some embodiments, the multispecific antibody comprises a first binding domain that binds to a TRDV2 antigen. In some embodiments, the multispecific antibody comprises a first binding domain that binds to a TRDV2 antigen and comprises a second binding domain that binds to a second target antigen, as provided herein. In certain embodiments, the multispecific antibody binds to a TRDV2 antigen, a second target antigen, and one or more additional antigens. In some embodiments of the various antibodies provided herein, the antibody binds to an epitope of a given antigen.

In some embodiments, a TRGDC antibody provided herein is chimeric. In some embodiments, a TRGDC antibody provided herein is human. In some embodiments, a TRGDC antibody provided herein is humanized. In certain embodiments, a TRGDC antibody provided herein is an isolated TRGDC antibody. In some embodiments, a TRGDC antigen binding fragment provided herein is chimeric. In some embodiments, a TRGDC antigen binding fragment provided herein is human. In some embodiments, a TRGDC antigen binding fragment provided herein is humanized. In certain embodiments, a TRGDC antigen binding fragment provided herein is an isolated TRGDC antigen binding fragment. In some embodiments, a TRGDC antibody provided herein is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody. In some embodiments, a TRGDC antibody provided herein is multivalent. In some embodiments, the antibody is capable of binding at least three antigens. In some embodiments, the antibody is capable of binding at least four antigens. In some embodiments, the antibody is capable of binding at least five antigens.

In some embodiments, a TRGDC multispecific antibody provided herein is chimeric. In some embodiments, a TRGDC multispecific antibody provided herein is human. In some embodiments, a TRGDC multispecific antibody provided herein is humanized. In certain embodiments, a TRGDC multispecific antibody provided herein is an isolated TRGDC multispecific antibody. In some embodiments, a TRGDC multispecific antibody comprising a TRGDC antigen binding fragment provided herein is chimeric. In some embodiments, a TRGDC multispecific antibody comprising a TRGDC antigen binding fragment provided herein is human. In some embodiments, a TRGDC multispecific antibody comprising a TRGDC antigen binding fragment provided herein is humanized. In certain embodiments, a TRGDC multispecific antibody comprising a TRGDC antigen binding fragment provided herein is an isolated TRGDC multispecific antibody.

In some embodiments of the TRGDC multispecific antibodies provided herein, the first binding domain is human. In some embodiments, the second binding domain is human. In some embodiments of the TRGDC multispecific antibodies provided herein, both the first binding domain and the second binding domain are human. In some embodiments of the TRGDC multispecific antibodies provided herein, the first binding domain is humanized. In some embodiments of the TRGDC multispecific antibodies provided herein, the second binding domain is humanized. In some embodiments of the TRGDC multispecific antibodies provided herein, both the first binding domain and the second binding domain are humanized. In some embodiments of the TRGDC multispecific antibodies provided herein, both the first binding domain is human and the second binding domain is humanized. In some embodiments of the TRGDC multispecific antibodies provided herein, both the first binding domain is humanized and the second binding domain is human.

In some embodiments, a TRGDC multispecific antibody provided herein is multivalent. In some embodiments, the multispecific antibody is capable of binding at least three antigens. In some embodiments, the multispecific antibody is capable of binding at least five antigens. In certain embodiments, the multispecific antibody is a multispecific antibody. In some embodiments, a TRGDC multispecific antibody provided herein is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody.

In certain embodiments, the antibodies provided herein are part of a multispecific antibody. In some embodiments, the multispecific antibody comprises a first binding domain that binds to a TRGDC antigen. In some embodiments, the multispecific antibody comprises a first binding domain that binds to a TRGDC antigen and comprises a second binding domain that binds to a second target antigen, as provided herein. In certain embodiments, the multispecific antibody binds to a TRGDC antigen, a second target antigen, and one or more additional antigens. In some embodiments of the various antibodies provided herein, the antibody binds to an epitope of a given antigen.

Also provided is a nucleic acid sequence encoding an antibody provided herein. In another general aspect, provide is a vector comprising an isolated nucleic acid sequence encoding an antibody provided herein. In another general aspect, provided is a vector comprising an isolated nucleic acid sequence encoding an antibody provided herein. Also provided is a vector comprising a nucleic acid sequence encoding an antibody provided herein. Also provided is a host cell comprising a vector comprising a nucleic acid sequence encoding an antibody provided herein. Also provided is a kit comprising the vector comprising a nucleic acid sequence encoding an antibody provided herein, and packaging for the same. In another general aspect, provided herein is an isolated nucleic acid sequence encoding a monoclonal antibody or antigen-binding fragment thereof provided herein. In certain embodiments, the antibody is a TRGV9 antibody. In certain embodiments, the antibody is a TRDV2 antibody. In certain embodiments, the antibody is a TRGDC antibody.

It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding antibodies provided herein can be altered without changing the amino acid sequences of the proteins.

Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to certain embodiments. Such techniques are well known to those skilled in the art in view of the present disclosure.

Also provided is a host cell comprising an isolated nucleic acid sequence encoding an antibody provided herein. Also provided is a host cell comprising an isolated nucleic acid sequence encoding an antigen binding fragment provided herein. Any host cell known to those skilled in the art in view of the present disclosure can be used for recombinant expression of antibodies or antigen-binding fragments thereof provided herein. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid sequence is effectively expressed.

Also provided are methods of producing an antibody disclosed herein. The methods comprise culturing a cell comprising a nucleic acid encoding the antibody under conditions to produce an antibody and recovering the antibody from the cell or cell culture (e.g., from the supernatant). Expressed antibodies can be harvested from the cells and purified according to conventional techniques known in the art and as described herein.

### Pharmaceutical Compositions

In another general aspect, provided is a pharmaceutical composition comprising a TRGV9 antibody provided herein and a pharmaceutically acceptable carrier. In another general aspect, provided is a pharmaceutical composition comprising a TRDV2 antibody provided herein and a pharmaceutically acceptable carrier. In another general aspect, provided is a pharmaceutical composition comprising a TRGDC antibody provided herein and a pharmaceutically acceptable carrier. In certain embodiments, the antibody is isolated. Also provided is a method of producing the pharmaceutical composition, comprising combining the antibody with a pharmaceutically acceptable carrier to obtain the pharmaceutical composition.

In another aspect, provided herein is a pharmaceutical composition comprising a comprising: (a) a first binding domain that binds to TRGV9, and (b) a second binding domain that binds to a second target, and a pharmaceutically acceptable carrier. In another aspect, provided herein is a pharmaceutical composition comprising a comprising: (a) a first binding domain that binds to TRDV2, and (b) a second binding domain that binds to a second target, and a pharmaceutically acceptable carrier. In another aspect, provided herein is a pharmaceutical composition comprising a comprising: (a) a first binding domain that binds to TRGDC, and (b) a second binding domain that binds to a second target, and a pharmaceutically acceptable carrier. Any of the multispecific antibodies provided herein are contemplated in the pharmaceutical compositions.

The term “pharmaceutical composition” as used herein means a product comprising an antibody provided herein together with a pharmaceutically acceptable carrier. Antibodies of provided herein and compositions comprising them are also useful in the manufacture of a medicament for therapeutic applications.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition provided herein the biological activity of a composition provided herein. According to particular embodiments, in view of the present disclosure, any pharmaceutically acceptable carrier suitable for use in an antibody pharmaceutical composition can be used herein.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carriers can be used in formulating the pharmaceutical compositions provided herein.

In one embodiment, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment, the pH of the formulation is from about 7.0 to about 9.5. In another embodiment, the pH of the formulation is from about 3.0 to about 7.0.

In another embodiment, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments.

In another embodiment, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments.

In another embodiment, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g. PEG400), and mixtures thereof. Another example of an isotonic agent includes a sugar. Non-limiting examples of sugars can include mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethyl-cellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments.

In another embodiment, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments.

In another embodiment, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment, the pharmaceutical composition comprises a stabilizer, wherein said stabilizer is carboxy-/hydroxycellulose and derivates thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments.

In further embodiments, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments.

In a further embodiment, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments.

In another general aspect, provided herein is a method of producing a pharmaceutical composition comprising an antibody or antigen-binding fragment thereof provided herein, comprising combining an antibody or antigen-binding fragment thereof with a pharmaceutically acceptable carrier to obtain the pharmaceutical composition.

### Methods of Use

The functional activity of antibodies provided herein can be characterized by methods known in the art and as described herein. Methods for characterizing antibodies and antigen-binding fragments thereof include, but are not limited to, affinity and specificity assays including Biacore, ELISA, and OctetRed analysis; binding assays to detect the binding of antibodies to target cells by FACS; binding assays to detect the binding of antibodies to the target antigen on cells. According to particular embodiments, the methods for characterizing antibodies and antigen-binding fragments thereof include those described below. In certain embodiments, the antibody is a TRGV9 antibody. In certain embodiments, the antibody is a TRDV2 antibody. In certain embodiments, the antibody is a TRGDC antibody.

In one aspect, provided is a method of activating a T cell expressing TRGV9, comprising contacting the T cell with a TRGV9 antibody provided herein. In some embodiments, the contacting results in an increase in CD69, CD25, and/or Granzyme B expression, as compared to a control T cell expressing TRGV9.

In another general aspect, provided is a method of inactivating a T cell expressing TRGV9, comprising contacting the T cell with an antibody that binds to a TRGV9 provided herein. In another general aspect, provided is a method of blocking activation of a T cell expressing TRGV9, comprising contacting the T cell with an antibody that binds to a TRGV9 provided herein. In another general aspect, provided is a method of modulating the activation of a T cell expressing TRGV9, comprising contacting the T cell with an antibody that binds to a TRGV9 provided herein.

In another aspect, provided herein is a method of directing a T cell expressing TRGV9 to a target cell, the method comprising contacting the T cell with a multispecific TRGV9 antibody provided herein. In another aspect, provided herein is a method of directing a T cell expressing TRGV9 to a target cell, the method comprising contacting the T cell with a pharmaceutical composition comprising a multispecific TRGV9 antibody provided herein. In some embodiments, the contacting directs the T cell to the target cell. Also provided is a method of targeting an antigen on the surface of a target cell, the method comprising exposing the target cell to a TRGV9 multispecific antibody provided herein. Also provided is a method of targeting an antigen on the surface of a target cell, the method comprising exposing the target cell to a pharmaceutical composition comprising a TRGV9 multispecific antibody provided herein. In another general aspect, provided is a method of targeting an antigen on the surface of a target cell, the method comprising exposing the target cell to a TRGV9 multispecific antibody provided herein. In another general aspect, provided is a method of targeting an antigen on the surface of a target cell, the method comprising exposing the target cell to a pharmaceutical composition comprising a TRGV9 multispecific antibody provided herein.

Also provided herein is a method of treating a disease or disorder in a subject, comprising administering to the subject a TRGV9 antibody provided herein. Also provided herein is a method of treating a disease or disorder in a subject, comprising administering to the subject a TRGV9 antigen binding fragment provided herein. Also provided herein is a method of treating a disease or disorder in a subject, comprising administering to the subject a pharmaceutical composition comprising a TRGV9 antibody provided herein. Also provided herein is a method of treating a disease or disorder in a subject, comprising administering to the subject a pharmaceutical composition comprising a TRGV9 antigen binding fragment provided herein. In a specific embodiment, the disease or disorder is caused all or in part by target cells expressing the second target.

In some embodiments, also provided is a method of directing a γδ T cell to a target cell, the method comprising contacting the γδ T cell with a multispecific TRGV9 antibody provided herein. In some embodiments, also provided is a TRGV9 antibody provided herein for use in directing a γδ T cell to a target cell. In some embodiments, also provided is a TRGV9 multispecific antibody provided herein for use in the manufacture of a medicament for directing a γδ T cell to a target cell. In certain embodiments, the target cell expresses the second target. In some embodiments, the contacting directs the γδ T cell to the target cell.

In some embodiments, also provided is a method of inhibiting growth or proliferation of target cells, the method comprising contacting the target cells with a multispecific TRGV9 antibody provided herein. In some embodiments, also provided is a TRGV9 multispecific antibody provided herein for use in inhibiting growth or proliferation of target cells. In some embodiments, also provided is a TRGV9 multispecific antibody provided herein for use in the manufacture of a medicament for inhibiting growth or proliferation of target cells. In certain embodiments, the target cells express the second target on the cell surface. In specific embodiments, the contacting the target cells with the multispecific TRGV9 antibody inhibits growth or proliferation of the target cells. In certain embodiments, the target cells are in the presence of a T cell expressing TRGV9 while in contact with the multispecific antibody. In a specific embodiment, the T cell expressing TRGV9 is a γδ T cell.

In some embodiments, also provided is a method for eliminating target cells in a subject, comprising administering an effective amount of a multispecific TRGV9 antibody provided herein to the subject. In some embodiments, also provided is a multispecific TRGV9 antibody provided herein for use in eliminating target cells in a subject. In some embodiments, also provided is a TRGV9 multispecific antibody provided herein for use in the manufacture of a medicament for the elimination of target cells in a subject. In some embodiments, the target cells express the second target on the cell surface. In certain embodiments, the target cells are in the presence of a T cell expressing TRGV9 in the subject. In a specific embodiment, the T cell expressing TRGV9 is a γδ T cell. In a specific embodiment, the subject is a subject in need thereof.

In some embodiments, also is a method for treating a disease caused all or in part by target cells in a subject, comprising administering an effective amount of a multispecific TRGV9 antibody provided herein to the subject. In some embodiments, also is a TRGV9 multispecific antibody for use in the treatment of a disease caused all or in part by target cells in a subject. In some embodiments, also provided is a TRGV9 multispecific antibody provided herein for use in the manufacture of a medicament for the treatment of a disease caused all or in part by target cells in a subject. In some embodiments, the target cells express the second target on the cell surface. In certain embodiments, the target cells are in the presence of a T cell expressing TRGV9 in the subject. In a specific embodiment, the T cell expressing TRGV9 is a γδ T cell. In a specific embodiment, the subject is a subject in need thereof. In some embodiments, the second target is a cancer antigen. In some embodiments, the second target is a tumor associated antigen.

Also provided is a method of activating a T cell expressing TRDV2, comprising contacting the T cell with a TRDV2 antibody provided herein. In some embodiments, the contacting results in an increase in CD69, CD25, and/or Granzyme B expression, as compared to a control T cell expressing TRDV2.

In another general aspect, provided is a method of inactivating a T cell expressing TRDV2, comprising contacting the T cell with an antibody that binds to a TRDV2 provided herein. In another general aspect, provided is a method of blocking activation of a T cell expressing TRDV2, comprising contacting the T cell with an antibody that binds to a TRDV2 provided herein. In another general aspect, provided is a method of modulating the activation of a T cell expressing TRDV2, comprising contacting the T cell with an antibody that binds to a TRDV2 provided herein.

In another aspect, provided herein is a method of directing a T cell expressing TRDV2 to a target cell, the method comprising contacting the T cell with a multispecific TRDV2 antibody provided herein. In another aspect, provided herein is a method of directing a T cell expressing TRDV2 to a target cell, the method comprising contacting the T cell with a pharmaceutical composition comprising a multispecific TRDV2 antibody provided herein. In some embodiments, the contacting directs the T cell to the target cell. Also provided is a method of targeting an antigen on the surface of a target cell, the method comprising exposing the target cell to a TRDV2 multispecific antibody provided herein. Also provided is a method of targeting an antigen on the surface of a target cell, the method comprising exposing the target cell to a pharmaceutical composition comprising a TRDV2 multispecific antibody provided herein. In another general aspect, provided is a method of targeting an antigen on the surface of a target cell, the method comprising exposing the target cell to a TRDV2 multispecific antibody provided herein. In another general aspect, provided is a method of targeting an antigen on the surface of a target cell, the method comprising exposing the target cell to a pharmaceutical composition comprising a TRDV2 multispecific antibody provided herein.

Also provided herein is a method of treating a disease or disorder in a subject, comprising administering to the subject a TRDV2 antibody provided herein. Also provided herein is a method of treating a disease or disorder in a subject, comprising administering to the subject a TRDV2 antigen binding fragment provided herein. Also provided herein is a method of treating a disease or disorder in a subject, comprising administering to the subject a pharmaceutical composition comprising a TRDV2 antibody provided herein. Also provided herein is a method of treating a disease or disorder in a subject, comprising administering to the subject a pharmaceutical composition comprising a TRDV2 antigen binding fragment provided herein. In a specific embodiment, the disease or disorder is caused all or in part by target cells expressing the second target.

In some embodiments, also provided is a method of directing a γδ T cell to a target cell, the method comprising contacting the γδ T cell with a multispecific TRDV2 antibody provided herein. In some embodiments, also provided is a TRDV2 antibody provided herein for use in directing a γδ T cell to a target cell. In some embodiments, also provided is a TRDV2 multispecific antibody provided herein for use in the manufacture of a medicament for directing a γδ T cell to a target cell. In certain embodiments, the target cell expresses the second target. In some embodiments, the contacting directs the γδ T cell to the target cell.

In some embodiments, also provided is a method of inhibiting growth or proliferation of target cells, the method comprising contacting the target cells with a multispecific TRDV2 antibody provided herein. In some embodiments, also provided is a TRDV2 multispecific antibody provided herein for use in inhibiting growth or proliferation of target cells. In some embodiments, also provided is a TRDV2 multispecific antibody provided herein for use in the manufacture of a medicament for inhibiting growth or proliferation of target cells. In certain embodiments, the target cells express the second target on the cell surface. In specific embodiments, the contacting the target cells with the multispecific TRDV2 antibody inhibits growth or proliferation of the target cells. In certain embodiments, the target cells are in the presence of a T cell expressing TRDV2 while in contact with the multispecific antibody. In a specific embodiment, the T cell expressing TRDV2 is a γδ T cell.

In some embodiments, also provided is a method for eliminating target cells in a subject, comprising administering an effective amount of a multispecific TRDV2 antibody provided herein to the subject. In some embodiments, also provided is a multispecific TRDV2 antibody provided herein for use in eliminating target cells in a subject. In some embodiments, also provided is a TRDV2 multispecific antibody provided herein for use in the manufacture of a medicament for the elimination of target cells in a subject. In some embodiments, the target cells express the second target on the cell surface. In certain embodiments, the target cells are in the presence of a T cell expressing TRDV2 in the subject. In a specific embodiment, the T cell expressing TRDV2 is a γδ T cell. In a specific embodiment, the subject is a subject in need thereof.

In some embodiments, also is a method for treating a disease caused all or in part by target cells in a subject, comprising administering an effective amount of a multispecific TRDV2 antibody provided herein to the subject. In some embodiments, also is a TRDV2 multispecific antibody for use in the treatment of a disease caused all or in part by target cells in a subject. In some embodiments, also provided is a TRDV2 multispecific antibody provided herein for use in the manufacture of a medicament for the treatment of a disease caused all or in part by target cells in a subject. In some embodiments, the target cells express the second target on the cell surface. In certain embodiments, the target cells are in the presence of a T cell expressing TRDV2 in the subject. In a specific embodiment, the T cell expressing TRDV2 is a γδ T cell. In a specific embodiment, the subject is a subject in need thereof. In some embodiments, the second target is a cancer antigen. In some embodiments, the second target is a tumor associated antigen.

Also provided is a method of activating a T cell expressing TRGDC, comprising 20 contacting the T cell with a TRGDC antibody provided herein. In some embodiments, the contacting results in an increase in CD69, CD25, and/or Granzyme B expression, as compared to a control T cell expressing TRGDC.

In another general aspect, provided is a method of inactivating a T cell expressing TRGDC, comprising contacting the T cell with an antibody that binds to a TRGDC provided herein. In another general aspect, provided is a method of blocking activation of a T cell expressing TRGDC, comprising contacting the T cell with an antibody that binds to a TRGDC provided herein. In another general aspect, provided is a method of modulating the activation of a T cell expressing TRGDC, comprising contacting the T cell with an antibody that binds to a TRGDC provided herein.

In another aspect, provided herein is a method of directing a T cell expressing TRGDC to a target cell, the method comprising contacting the T cell with a multispecific TRGDC antibody provided herein. In another aspect, provided herein is a method of directing a T cell expressing TRGDC to a target cell, the method comprising contacting the T cell with a pharmaceutical composition comprising a multispecific TRGDC antibody provided herein. In some embodiments, the contacting directs the T cell to the target cell. Also provided is a method of targeting an antigen on the surface of a target cell, the method comprising exposing the target cell to a TRGDC multispecific antibody provided herein. Also provided is a method of targeting an antigen on the surface of a target cell, the method comprising exposing the target cell to a pharmaceutical composition comprising a TRGDC multispecific antibody provided herein. In another general aspect, provided is a method of targeting an antigen on the surface of a target cell, the method comprising exposing the target cell to a TRGDC multispecific antibody provided herein. In another general aspect, provided is a method of targeting an antigen on the surface of a target cell, the method comprising exposing the target cell to a pharmaceutical composition comprising a TRGDC multispecific antibody provided herein.

Also provided herein is a method of treating a disease or disorder in a subject, comprising administering to the subject a TRGDC antibody provided herein. Also provided herein is a method of treating a disease or disorder in a subject, comprising administering to the subject a TRGDC antigen binding fragment provided herein. Also provided herein is a method of treating a disease or disorder in a subject, comprising administering to the subject a pharmaceutical composition comprising a TRGDC antibody provided herein. Also provided herein is a method of treating a disease or disorder in a subject, comprising administering to the subject a pharmaceutical composition comprising a TRGDC antigen binding fragment provided herein. In a specific embodiment, the disease or disorder is caused all or in part by target cells expressing the second target.

In some embodiments, also provided is a method of directing a γδ T cell to a target cell, the method comprising contacting the γδ T cell with a multispecific TRGDC antibody provided herein. In some embodiments, also provided is a TRGDC antibody provided herein for use in directing a γδ T cell to a target cell. In some embodiments, also provided is a TRGDC multispecific antibody provided herein for use in the manufacture of a medicament for directing a γδ T cell to a target cell. In certain embodiments, the target cell expresses the second target. In some embodiments, the contacting directs the γδ T cell to the target cell.

In some embodiments, also provided is a method of inhibiting growth or proliferation of target cells, the method comprising contacting the target cells with a multispecific TRGDC antibody provided herein. In some embodiments, also provided is a TRGDC multispecific antibody provided herein for use in inhibiting growth or proliferation of target cells. In some embodiments, also provided is a TRGDC multispecific antibody provided herein for use in the manufacture of a medicament for inhibiting growth or proliferation of target cells. In certain embodiments, the target cells express the second target on the cell surface. In specific embodiments, the contacting the target cells with the multispecific TRGDC antibody inhibits growth or proliferation of the target cells. In certain embodiments, the target cells are in the presence of a T cell expressing TRGDC while in contact with the multispecific antibody. In a specific embodiment, the T cell expressing TRGDC is a γδ T cell.

In some embodiments, also provided is a method for eliminating target cells in a subject, comprising administering an effective amount of a multispecific TRGDC antibody provided herein to the subject. In some embodiments, also provided is a multispecific TRGDC antibody provided herein for use in eliminating target cells in a subject. In some embodiments, also provided is a TRGDC multispecific antibody provided herein for use in the manufacture of a medicament for the elimination of target cells in a subject. In some embodiments, the target cells express the second target on the cell surface. In certain embodiments, the target cells are in the presence of a T cell expressing TRGDC in the subject. In a specific embodiment, the T cell expressing TRGDC is a γδ T cell. In a specific embodiment, the subject is a subject in need thereof.

In some embodiments, also is a method for treating a disease caused all or in part by target cells in a subject, comprising administering an effective amount of a multispecific TRGDC antibody provided herein to the subject. In some embodiments, also is a TRGDC multispecific antibody for use in the treatment of a disease caused all or in part by target cells in a subject. In some embodiments, also provided is a TRGDC multispecific antibody provided herein for use in the manufacture of a medicament for the treatment of a disease caused all or in part by target cells in a subject. In some embodiments, the target cells express the second target on the cell surface. In certain embodiments, the target cells are in the presence of a T cell expressing TRGDC in the subject. In a specific embodiment, the T cell expressing TRGDC is a γδ T cell. In a specific embodiment, the subject is a subject in need thereof. In some embodiments, the second target is a cancer antigen. In some embodiments, the second target is a tumor associated antigen.

In some embodiments, the subject is a subject in need thereof. In some embodiments, the subject is a human. In specific embodiments, the subject is administered an effective amount.

As used herein, the term “effective amount” refers to an amount of an active ingredient or component that elicits the desired biological or medicinal response in a subject.

According to particular embodiments, an effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

In some embodiments, a TRGV9 antibody provided herein is used in combination with a supplemental therapy. In some embodiments, a TRDV2 antibody provided herein is used in combination with a supplemental therapy.

In some embodiments, a TRGDC antibody provided herein is used in combination with a supplemental therapy.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

TRGV9 antibodies provided herein may also be used as agents to detect TRGV9-expressing cells. Thus, in another methods, provided is a method of detecting a cell expressing TRGV9, comprising contacting a cell with a TRGV9 antibody provided herein. In certain embodiments, the detecting is by ELISA. In some embodiments, the detecting is by FACS analysis. Also provided are kits comprising a TRGV9 antibody provided herein, and instructions for use.

TRDV2 antibodies provided herein may also be used as agents to detect TRDV2-expressing cells. Thus, in another methods, provided is a method of detecting a cell expressing TRDV2, comprising contacting a cell with a TRDV2 antibody provided herein. In certain embodiments, the detecting is by ELISA. In some embodiments, the detecting is by FACS analysis. Also provided are kits comprising a TRDV2 antibody provided herein, and instructions for use.

TRGDC antibodies provided herein may also be used as agents to detect TRGDC-expressing cells. Thus, in another methods, provided is a method of detecting a cell expressing TRGDC, comprising contacting a cell with a TRGDC antibody provided herein. In certain embodiments, the detecting is by ELISA. In some embodiments, the detecting is by FACS analysis. Also provided are kits comprising a TRGDC antibody provided herein, and instructions for use.

### Enrichment and Detection Methods

In one aspect, the TRGV9 antibodies provided herein are used as agents to detect TRGV9-expressing cells. Thus, in other methods, provided is a method of detecting a cell expressing TRGV9, comprising contacting a cell with a TRGV9 antibody provided herein. In certain embodiments, the detecting is by ELISA. In some embodiments, the detecting is by FACS analysis. Also provided are kits comprising a TRGV9 antibody provided herein, and instructions for use.

Enrichment, isolation, separation, purification, sorting, selecting, capturing or detecting, or any combination thereof can be done using known technologies such as bead, microfluidics, solid support, columns, and the like. For example, TRGV9 cells may be separated or visualized using known methods when bound to the TRGV9 antibodies provided herein.

The TRGV9 antibodies or multispecific TRGV9 antibodies provided herein can be used to selectively enrich, isolate, separate, purify, sort, select, capture or detect TRGV9-expressing cells. The TRGV9 antibodies or multispecific TRGV9 antibodies provided herein may be utilized in a bispecific format, e.g. containing a first antigen binding domain that specifically binds TRGV9 and a second antigen binding domain that specifically binds a second target. In other embodiments, the multispecific TRGV9 antibodies provided herein may be utilized in a format that further incorporates a third antigen binding domain that specifically binds a third antigen (e.g., at a trispecific antibody). In other embodiments, the multispecific TRGV9 antibodies provided herein may be utilized in a format that further incorporates a fourth antigen binding domain that specifically binds a fourth antigen. (e.g., as a quadraspecific antibody).

In one aspect, provided herein is a method of enriching a TRGV9-expressing cell comprising: providing a sample comprising the TRGV9-expressing cell; contacting the sample with a TRGV9 antibody provided herein; and enriching the TRGV9-expressing cell bound to the TRGV9 antibody. In one aspect, provided herein is a method of isolating a TRGV9-expressing cell comprising: providing a sample comprising the TRGV9-expressing cell; contacting the sample with a TRGV9 antibody provided herein; and isolating the TRGV9-expressing cell bound to the TRGV9 antibody. In one aspect, provided herein is a method of separating a TRGV9-expressing cell comprising: providing a sample comprising the TRGV9-expressing cell; contacting the sample with a TRGV9 antibody provided herein; and separating the TRGV9-expressing cell bound to the TRGV9 antibody. In one aspect, 10 provided herein is a method of purifying a TRGV9-expressing cell comprising: providing a sample comprising the TRGV9-expressing cell; contacting the sample with a TRGV9 antibody provided herein; and purifying the TRGV9-expressing cell bound to the TRGV9 antibody. In one aspect, provided herein is a method of sorting a TRGV9-expressing cell comprising: providing a sample comprising the TRGV9-expressing cell; contacting the sample with a TRGV9 antibody provided herein; and sorting the TRGV9-expressing cell bound to the TRGV9 antibody. In one aspect, provided herein is a method of selecting a TRGV9-expressing cell comprising: providing a sample comprising the TRGV9-expressing cell; contacting the sample with a TRGV9 antibody provided herein; and selecting the TRGV9-expressing cell bound to the TRGV9 antibody. In one aspect, provided herein is a method of capturing a TRGV9-expressing cell comprising: providing a sample comprising the TRGV9-expressing cell; contacting the sample with a TRGV9 antibody provided herein; and capturing the TRGV9-expressing cell bound to the TRGV9 antibody. In one aspect, provided herein is a method of detecting a TRGV9-expressing cell comprising: providing a sample comprising the TRGV9-expressing cell; contacting the sample with a TRGV9 antibody provided herein; and detecting the TRGV9-expressing cell bound to the TRGV9 antibody.

In one aspect, provided herein is a method of enriching a TRGV9-expressing cell comprising: contacting a TRGV9-expressing cell with a TRGV9 antibody provided herein; and enriching the TRGV9-expressing cell bound to the TRGV9 antibody. In one aspect, provided herein is a method of isolating a TRGV9-expressing cell comprising: contacting a TRGV9-expressing cell with a TRGV9 antibody provided herein; and isolating the TRGV9-expressing cell bound to the TRGV9 antibody. In one aspect, provided herein is a method of separating a TRGV9-expressing cell comprising: contacting a TRGV9-expressing cell with a TRGV9 antibody provided herein; and separating the TRGV9-expressing cell bound to the TRGV9 antibody. In one aspect, provided herein is a method of purifying a TRGV9-expressing cell comprising: contacting a TRGV9-expressing cell with a TRGV9 antibody provided herein; and purifying the TRGV9-expressing cell bound to the TRGV9 antibody. In one aspect, provided herein is a method of sorting a TRGV9-expressing cell comprising: contacting a TRGV9-expressing cell with a TRGV9 antibody provided herein; and sorting the TRGV9-expressing cell bound to the TRGV9 antibody. In one aspect, provided herein is a method of selecting a TRGV9-expressing cell comprising: contacting a TRGV9-expressing cell with a TRGV9 antibody provided herein; and selecting the TRGV9-expressing cell bound to the TRGV9 antibody. In one aspect, provided herein is a method of capturing a TRGV9-expressing cell comprising: contacting a TRGV9-expressing cell with a TRGV9 antibody provided herein; and capturing the TRGV9-expressing cell bound to the TRGV9 antibody. In one aspect, provided herein is a method of detecting a TRGV9-expressing cell comprising: contacting a TRGV9-expressing cell with a TRGV9 antibody provided herein; and detecting the TRGV9-expressing cell bound to the TRGV9 antibody.

In one aspect, provided herein is a method of enriching a TRGV9-expressing cell comprising: contacting a TRGV9-expressing cell with a TRGV9 antibody provided herein; and enriching the TRGV9-expressing cell based on binding of the TRGV9-expressing cell to the TRGV9 antibody. In one aspect, provided herein is a method of isolating a TRGV9-expressing cell comprising: contacting a TRGV9-expressing cell with a TRGV9 antibody provided herein; and isolating the TRGV9-expressing cell based on binding of the TRGV9-expressing cell to the TRGV9 antibody. In one aspect, provided herein is a method of separating a TRGV9-expressing cell comprising: contacting a TRGV9-expressing cell with a TRGV9 antibody provided herein; and separating the TRGV9-expressing cell based on binding of the TRGV9-expressing cell to the TRGV9 antibody. In one aspect, provided herein is a method of purifying a TRGV9-expressing cell comprising: contacting a TRGV9-expressing cell with a TRGV9 antibody provided herein; and purifying the TRGV9-expressing cell based on binding of the TRGV9-expressing cell to the TRGV9 antibody. In one aspect, provided herein is a method of sorting a TRGV9-expressing cell comprising: contacting a TRGV9-expressing cell with a TRGV9 antibody provided herein; and sorting the TRGV9-expressing cell based on binding of the TRGV9-expressing cell to the TRGV9 antibody. In one aspect, provided herein is a method of selecting a TRGV9-expressing cell comprising: contacting a TRGV9-expressing cell with a TRGV9 antibody provided herein; and selecting the TRGV9-expressing cell based on binding of the TRGV9-expressing cell to the TRGV9 antibody. In one aspect, provided herein is a method of capturing a TRGV9-expressing cell comprising: contacting a TRGV9-expressing cell with a TRGV9 antibody provided herein; and capturing the TRGV9-expressing cell based on binding of the TRGV9-expressing cell to the TRGV9 antibody. In one aspect, provided herein is a method of detecting a TRGV9-expressing cell comprising: contacting a TRGV9-expressing cell with a TRGV9 antibody provided herein; and detecting the TRGV9-expressing cell based on binding of the TRGV9-expressing cell to the TRGV9 antibody.

In certain embodiments of the methods, the TRGV9-expressing cell is a T cell. In some embodiments of the methods, the TRGV9-expressing cell is in a population of cells. In some embodiments of the methods, the TRGV9-expressing cell is in a population of lymphocytes. In some embodiments of the methods, the TRGV9-expressing cell is in a population of T cells. In some embodiments of the methods, the TRGV9-expressing cell is provided as a population of cells. In some embodiments of the methods, the TRGV9-expressing cell is provided as a population of lymphocytes. In some embodiments of the methods, the TRGV9-expressing cell is provided as a population of T cells. In some embodiments of the methods, the TRGV9-expressing cell is provided as a sample comprising a population of cells. In some embodiments of the methods, the TRGV9-expressing cell is provided as a sample comprising a population of lymphocytes. In some embodiments of the methods, the TRGV9-expressing cell is provided as a sample comprising a population of T cells. In some embodiments of the methods, the sample is a blood sample. In some embodiments of the methods, the sample is a tissue sample. In some embodiments of the methods, the sample is a tissue culture sample.

In some embodiments of the methods, the TRGV9 antibody is a multispecific TRGV9 antibody provided herein. In some embodiments of the methods, the TRGV9 antibody is a bispecific TRGV9 antibody provided herein. In some embodiments of the methods, the TRGV9 antibody is a trispecific TRGV9 antibody provided herein. In some embodiments of the methods, the TRGV9 antibody is a quadraspecific TRGV9 antibody provided herein. In certain embodiments, the TRGV9 antibody specifically binds to TRGV9. In one embodiment, the multispecific TRGV9 antibody comprises: (a) a first binding domain that binds TRGV9, and (b) a second binding domain that binds to a second target. In one embodiment, the multispecific TRGV9 antibody comprises: (a) a first binding domain that binds TRGV9, and (b) a second binding domain that binds to a second target, and (c) a third binding domain that binds to a third target. In one embodiment, the multispecific TRGV9 antibody comprises: (a) a first binding domain that binds TRGV9, and (b) a second binding domain that binds to a second target, (c) a third binding domain that binds to a third target, and (d) a fourth binding domain that binds to a fourth target. In one embodiment, the multispecific TRGV9 antibody comprises: (a) a first binding domain that specifically binds TRGV9, and (b) a second binding domain that specifically binds to a second target. In one embodiment, the multispecific TRGV9 antibody comprises: (a) a first binding domain that specifically binds TRGV9, and (b) a second binding domain that specifically binds to a second target, and (c) a third binding domain that specifically binds to a third target. In one embodiment, the multispecific TRGV9 antibody comprises: (a) a first binding domain that specifically binds TRGV9, and (b) a second binding domain that specifically binds to a second target, (c) a third binding domain that specifically binds to a third target, and (d) a fourth binding domain that specifically binds to a fourth target.

In specific embodiments of the methods provided herein, the method uses multi-marker detection. In some embodiments, the multi-marker detection uses a multispecific TRGV9 antibody provided herein. In some embodiments, the multi-marker detection uses a bispecific TRGV9 antibody provided herein. In some embodiments, the multi-marker detection uses a trispecific TRGV9 antibody provided herein. In some embodiments, the multi-marker detection uses a quadraspecific TRGV9 antibody provided herein.

In certain embodiments of the methods provided herein, the methods are included as steps in a T cell manufacturing process. In certain embodiments, the cells are CAR-T cells. In certain embodiments of the methods provided herein, the methods are included as steps in a T cell modification process.

In certain embodiments of the methods provided herein, the methods are included as steps in a diagnostic method. In certain embodiments of the methods provided herein, the methods are included as steps in a method to quantify the TRGV9-expressing T cells.

In certain embodiments of the methods provided herein, the method further comprises expanding the enriched, isolated, separated, purified, sorted, selected, captured or detected TRGV9-expressing cells. In certain embodiments, the expanding is in vitro. In certain embodiments, the expanding is in vivo. In certain embodiments of the methods provided herein, the method further comprises growing the enriched, isolated, separated, purified, sorted, selected, captured or detected TRGV9-expressing cells. In certain embodiments, the growing is in vitro. In certain embodiments, the growing is in vivo. In certain embodiments of the methods provided herein, the method further comprises quantifying the enriched, isolated, separated, purified, sorted, selected, captured or detected TRGV9-expressing cells.

In one aspect, the TRDV2 antibodies provided herein are used as agents to detect TRDV2-expressing cells. Thus, in other methods, provided is a method of detecting a cell expressing TRDV2, comprising contacting a cell with a TRDV2 antibody provided herein. In certain embodiments, the detecting is by ELISA. In some embodiments, the detecting is by FACS analysis. Also provided are kits comprising a TRDV2 antibody provided herein, and instructions for use.

Enrichment, isolation, separation, purification, sorting, selecting, capturing or detecting, or any combination thereof can be done using known technologies such as bead, microfluidics, solid support, columns, and the like. For example, TRDV2 cells may be separated or visualized using known methods when bound to the TRDV2 antibodies provided herein.

The TRDV2 antibodies or multispecific TRDV2 antibodies provided herein can be used to selectively enrich, isolate, separate, purify, sort, select, capture or detect TRDV2-expressing cells. The TRDV2 antibodies or multispecific TRDV2 antibodies provided herein may be utilized in a bispecific format, e.g. containing a first antigen binding domain that specifically binds TRDV2 and a second antigen binding domain that specifically binds a second target. In other embodiments, the multispecific TRDV2 antibodies provided herein may be utilized in a format that further incorporates a third antigen binding domain that specifically binds a third antigen (e.g., at a trispecific antibody). In other embodiments, the multispecific TRDV2 antibodies provided herein may be utilized in a format that further incorporates a fourth antigen binding domain that specifically binds a fourth antigen. (e.g., as a quadraspecific antibody).

In one aspect, provided herein is a method of enriching a TRDV2-expressing cell comprising: providing a sample comprising the TRDV2-expressing cell; contacting the sample with a TRDV2 antibody provided herein; and enriching the TRDV2-expressing cell bound to the TRDV2 antibody. In one aspect, provided herein is a method of isolating a TRDV2-expressing cell comprising: providing a sample comprising the TRDV2-expressing cell; contacting the sample with a TRDV2 antibody provided herein; and isolating the TRDV2-expressing cell bound to the TRDV2 antibody. In one aspect, provided herein is a method of separating a TRDV2-expressing cell comprising: providing a sample comprising the TRDV2-expressing cell; contacting the sample with a TRDV2 antibody provided herein; and separating the TRDV2-expressing cell bound to the TRDV2 antibody. In one aspect, provided herein is a method of purifying a TRDV2-expressing cell comprising: providing a sample comprising the TRDV2-expressing cell; contacting the sample with a TRDV2 antibody provided herein; and purifying the TRDV2-expressing cell bound to the TRDV2 antibody. In one aspect, provided herein is a method of sorting a TRDV2-expressing cell comprising: providing a sample comprising the TRDV2-expressing cell; contacting the sample with a TRDV2 antibody provided herein; and sorting the TRDV2-expressing cell bound to the TRDV2 antibody. In one aspect, provided herein is a method of selecting a TRDV2-expressing cell comprising: providing a sample comprising the TRDV2-expressing cell; contacting the sample with a TRDV2 antibody provided herein; and selecting the TRDV2-expressing cell bound to the TRDV2 antibody. In one aspect, provided herein is a method of capturing a TRDV2-expressing cell comprising: providing a sample comprising the TRDV2-expressing cell; contacting the sample with a TRDV2 antibody provided herein; and capturing the TRDV2-expressing cell bound to the TRDV2 antibody. In one aspect, provided herein is a method of detecting a TRDV2-expressing cell comprising: providing a sample comprising the TRDV2-expressing cell; contacting the sample with a TRDV2 antibody provided herein; and detecting the TRDV2-expressing cell bound to the TRDV2 antibody.

In one aspect, provided herein is a method of enriching a TRDV2-expressing cell comprising: contacting a TRDV2-expressing cell with a TRDV2 antibody provided herein; and enriching the TRDV2-expressing cell bound to the TRDV2 antibody. In one aspect, provided herein is a method of isolating a TRDV2-expressing cell comprising: contacting a TRDV2-expressing cell with a TRDV2 antibody provided herein; and isolating the TRDV2-expressing cell bound to the TRDV2 antibody. In one aspect, provided herein is a method of separating a TRDV2-expressing cell comprising: contacting a TRDV2-expressing cell with a TRDV2 antibody provided herein; and separating the TRDV2-expressing cell bound to the TRDV2 antibody. In one aspect, provided herein is a method of purifying a TRDV2-expressing cell comprising: contacting a TRDV2-expressing cell with a TRDV2 antibody provided herein; and purifying the TRDV2-expressing cell bound to the TRDV2 antibody. In one aspect, provided herein is a method of sorting a TRDV2-expressing cell comprising: contacting a TRDV2-expressing cell with a TRDV2 antibody provided herein; and sorting the TRDV2-expressing cell bound to the TRDV2 antibody. In one aspect, provided herein is a method of selecting a TRDV2-expressing cell comprising: contacting a TRDV2-expressing cell with a TRDV2 antibody provided herein; and selecting the TRDV2-expressing cell bound to the TRDV2 antibody. In one aspect, provided herein is a method of capturing a TRDV2-expressing cell comprising: contacting a TRDV2-expressing cell with a TRDV2 antibody provided herein; and capturing the TRDV2-expressing cell bound to the TRDV2 antibody. In one aspect, provided herein is a method of detecting a TRDV2-expressing cell comprising: contacting a TRDV2-expressing cell with a TRDV2 antibody provided herein; and detecting the TRDV2-expressing cell bound to the TRDV2 antibody.

In one aspect, provided herein is a method of enriching a TRDV2-expressing cell comprising: contacting a TRDV2-expressing cell with a TRDV2 antibody provided herein; and enriching the TRDV2-expressing cell based on binding of the TRDV2-expressing cell to the TRDV2 antibody. In one aspect, provided herein is a method of isolating a TRDV2-expressing cell comprising: contacting a TRDV2-expressing cell with a TRDV2 antibody provided herein; and isolating the TRDV2-expressing cell based on binding of the TRDV2-expressing cell to the TRDV2 antibody. In one aspect, provided herein is a method of separating a TRDV2-expressing cell comprising: contacting a TRDV2-expressing cell with a TRDV2 antibody provided herein; and separating the TRDV2-expressing cell based on binding of the TRDV2-expressing cell to the TRDV2 antibody. In one aspect, provided herein is a method of purifying a TRDV2-expressing cell comprising: contacting a TRDV2-expressing cell with a TRDV2 antibody provided herein; and purifying the TRDV2-expressing cell based on binding of the TRDV2-expressing cell to the TRDV2 antibody. In one aspect, provided herein is a method of sorting a TRDV2-expressing cell comprising: contacting a TRDV2-expressing cell with a TRDV2 antibody provided herein; and sorting the TRDV2-expressing cell based on binding of the TRDV2-expressing cell to the TRDV2 antibody. In one aspect, provided herein is a method of selecting a TRDV2-expressing cell comprising: contacting a TRDV2-expressing cell with a TRDV2 antibody provided herein; and selecting the TRDV2-expressing cell based on binding of the TRDV2-expressing cell to the TRDV2 antibody. In one aspect, provided herein is a method of capturing a TRDV2-expressing cell comprising: contacting a TRDV2-expressing cell with a TRDV2 antibody provided herein; and capturing the TRDV2-expressing cell based on binding of the TRDV2-expressing cell to the TRDV2 antibody. In one aspect, provided herein is a method of detecting a TRDV2-expressing cell comprising: contacting a TRDV2-expressing cell with a TRDV2 antibody provided herein; and detecting the TRDV2-expressing cell based on binding of the TRDV2-expressing cell to the TRDV2 antibody.

In certain embodiments of the methods, the TRDV2-expressing cell is a T cell. In some embodiments of the methods, the TRDV2-expressing cell is in a population of cells. In some embodiments of the methods, the TRDV2-expressing cell is in a population of lymphocytes. In some embodiments of the methods, the TRDV2-expressing cell is in a population of T cells. In some embodiments of the methods, the TRDV2-expressing cell is provided as a population of cells. In some embodiments of the methods, the TRDV2-expressing cell is provided as a population of lymphocytes. In some embodiments of the methods, the TRDV2-expressing cell is provided as a population of T cells. In some embodiments of the methods, the TRDV2-expressing cell is provided as a sample comprising a population of cells. In some embodiments of the methods, the TRDV2-expressing cell is provided as a sample comprising a population of lymphocytes. In some embodiments of the methods, the TRDV2-expressing cell is provided as a sample comprising a population of T cells. In some embodiments of the methods, the sample is a blood sample. In some embodiments of the methods, the sample is a tissue sample. In some embodiments of the methods, the sample is a tissue culture sample.

In some embodiments of the methods, the TRDV2 antibody is a multispecific TRDV2 antibody provided herein. In some embodiments of the methods, the TRDV2 antibody is a bispecific TRDV2 antibody provided herein. In some embodiments of the methods, the TRDV2 antibody is a trispecific TRDV2 antibody provided herein. In some embodiments of the methods, the TRDV2 antibody is a quadraspecific TRDV2 antibody provided herein. In certain embodiments, the TRDV2 antibody specifically binds to TRDV2. In one embodiment, the multispecific TRDV2 antibody comprises: (a) a first binding domain that binds TRDV2, and (b) a second binding domain that binds to a second target. In one embodiment, the multispecific TRDV2 antibody comprises: (a) a first binding domain that binds TRDV2, and (b) a second binding domain that binds to a second target, and (c) a third binding domain that binds to a third target. In one embodiment, the multispecific TRDV2 antibody comprises: (a) a first binding domain that binds TRDV2, and (b) a second binding domain that binds to a second target, (c) a third binding domain that binds to a third target, and (d) a fourth binding domain that binds to a fourth target. In one embodiment, the multispecific TRDV2 antibody comprises: (a) a first binding domain that specifically binds TRDV2, and (b) a second binding domain that specifically binds to a second target. In one embodiment, the multispecific TRDV2 antibody comprises: (a) a first binding domain that specifically binds TRDV2, and (b) a second binding domain that specifically binds to a second target, and (c) a third binding domain that specifically binds to a third target. In one embodiment, the multispecific TRDV2 antibody comprises: (a) a first binding domain that specifically binds TRDV2, and (b) a second binding domain that specifically binds to a second target, (c) a third binding domain that specifically binds to a third target, and (d) a fourth binding domain that specifically binds to a fourth target.

In specific embodiments of the methods provided herein, the method uses multi-marker detection. In some embodiments, the multi-marker detection uses a multispecific TRDV2 antibody provided herein. In some embodiments, the multi-marker detection uses a bispecific TRDV2 antibody provided herein. In some embodiments, the multi-marker detection uses a trispecific TRDV2 antibody provided herein. In some embodiments, the multi-marker detection uses a quadraspecific TRDV2 antibody provided herein.

In certain embodiments of the methods provided herein, the methods are included as steps in a T cell manufacturing process. In certain embodiments, the cells are CAR-T cells. In certain embodiments of the methods provided herein, the methods are included as steps in a T cell modification process.

In certain embodiments of the methods provided herein, the methods are included as steps in a diagnostic method. In certain embodiments of the methods provided herein, the methods are included as steps in a method to quantify the TRDV2-expressing T cells.

In certain embodiments of the methods provided herein, the method further comprises expanding the enriched, isolated, separated, purified, sorted, selected, captured or detected TRDV2-expressing cells. In certain embodiments, the expanding is in vitro. In certain embodiments, the expanding is in vivo. In certain embodiments of the methods provided herein, the method further comprises growing the enriched, isolated, separated, purified, sorted, selected, captured or detected TRDV2-expressing cells. In certain embodiments, the growing is in vitro. In certain embodiments, the growing is in vivo. In certain embodiments of the methods provided herein, the method further comprises quantifying the enriched, isolated, separated, purified, sorted, selected, captured or detected TRDV2-expressing cells.

In one aspect, the TRGDC antibodies provided herein are used as agents to detect TRGDC-expressing cells. Thus, in other methods, provided is a method of detecting a cell expressing TRGDC, comprising contacting a cell with a TRGDC antibody provided herein. In certain embodiments, the detecting is by ELISA. In some embodiments, the detecting is by FACS analysis. Also provided are kits comprising a TRGDC antibody provided herein, and instructions for use.

Enrichment, isolation, separation, purification, sorting, selecting, capturing or detecting, or any combination thereof can be done using known technologies such as bead, microfluidics, solid support, columns, and the like. For example, TRGDC cells may be separated or visualized using known methods when bound to the TRGDC antibodies provided herein.

The TRGDC antibodies or multispecific TRGDC antibodies provided herein can be used to selectively enrich, isolate, separate, purify, sort, select, capture or detect TRGDC-expressing cells. The TRGDC antibodies or multispecific TRGDC antibodies provided herein may be utilized in a bispecific format, e.g. containing a first antigen binding domain that specifically binds TRGDC and a second antigen binding domain that specifically binds a second target. In other embodiments, the multispecific TRGDC antibodies provided herein may be utilized in a format that further incorporates a third antigen binding domain that specifically binds a third antigen (e.g., at a trispecific antibody). In other embodiments, the multispecific TRGDC antibodies provided herein may be utilized in a format that further incorporates a fourth antigen binding domain that specifically binds a fourth antigen. (e.g., as a quadraspecific antibody).

In one aspect, provided herein is a method of enriching a TRGDC-expressing cell comprising: providing a sample comprising the TRGDC-expressing cell; contacting the sample with a TRGDC antibody provided herein; and enriching the TRGDC-expressing cell bound to the TRGDC antibody. In one aspect, provided herein is a method of isolating a TRGDC-expressing cell comprising: providing a sample comprising the TRGDC-expressing cell; contacting the sample with a TRGDC antibody provided herein; and isolating the TRGDC-expressing cell bound to the TRGDC antibody. In one aspect, provided herein is a method of separating a TRGDC-expressing cell comprising: providing a sample comprising the TRGDC-expressing cell; contacting the sample with a TRGDC antibody provided herein; and separating the TRGDC-expressing cell bound to the TRGDC antibody. In one aspect, provided herein is a method of purifying a TRGDC-expressing cell comprising: providing a sample comprising the TRGDC-expressing cell; contacting the sample with a TRGDC antibody provided herein; and purifying the TRGDC-expressing cell bound to the TRGDC antibody. In one aspect, provided herein is a method of sorting a TRGDC-expressing cell comprising: providing a sample comprising the TRGDC-expressing cell; contacting the sample with a TRGDC antibody provided herein; and sorting the TRGDC-expressing cell bound to the TRGDC antibody. In one aspect, provided herein is a method of selecting a TRGDC-expressing cell comprising: providing a sample comprising the TRGDC-expressing cell; contacting the sample with a TRGDC antibody provided herein; and selecting the TRGDC-expressing cell bound to the TRGDC antibody. In one aspect, provided herein is a method of capturing a TRGDC-expressing cell comprising: providing a sample comprising the TRGDC-expressing cell; contacting the sample with a TRGDC antibody provided herein; and capturing the TRGDC-expressing cell bound to the TRGDC antibody. In one aspect, provided herein is a method of detecting a TRGDC-expressing cell comprising: providing a sample comprising the TRGDC-expressing cell; contacting the sample with a TRGDC antibody provided herein; and detecting the TRGDC-expressing cell bound to the TRGDC antibody.

In one aspect, provided herein is a method of enriching a TRGDC-expressing cell comprising: contacting a TRGDC-expressing cell with a TRGDC antibody provided herein; and enriching the TRGDC-expressing cell bound to the TRGDC antibody. In one aspect, provided herein is a method of isolating a TRGDC-expressing cell comprising: contacting a TRGDC-expressing cell with a TRGDC antibody provided herein; and isolating the TRGDC-expressing cell bound to the TRGDC antibody. In one aspect, provided herein is a method of separating a TRGDC-expressing cell comprising: contacting a TRGDC-expressing cell with a TRGDC antibody provided herein; and separating the TRGDC-expressing cell bound to the TRGDC antibody. In one aspect, provided herein is a method of purifying a TRGDC-expressing cell comprising: contacting a TRGDC-expressing cell with a TRGDC antibody provided herein; and purifying the TRGDC-expressing cell bound to the TRGDC antibody. In one aspect, provided herein is a method of sorting a TRGDC-expressing cell comprising: contacting a TRGDC-expressing cell with a TRGDC antibody provided herein; and sorting the TRGDC-expressing cell bound to the TRGDC antibody. In one aspect, provided herein is a method of selecting a TRGDC-expressing cell comprising: contacting a TRGDC-expressing cell with a TRGDC antibody provided herein; and selecting the TRGDC-expressing cell bound to the TRGDC antibody. In one aspect, provided herein is a method of capturing a TRGDC-expressing cell comprising: contacting a TRGDC-expressing cell with a TRGDC antibody provided herein; and capturing the TRGDC-expressing cell bound to the TRGDC antibody. In one aspect, provided herein is a method of detecting a TRGDC-expressing cell comprising: contacting a TRGDC-expressing cell with a TRGDC antibody provided herein; and detecting the TRGDC-expressing cell bound to the TRGDC antibody.

In one aspect, provided herein is a method of enriching a TRGDC-expressing cell comprising: contacting a TRGDC-expressing cell with a TRGDC antibody provided herein; and enriching the TRGDC-expressing cell based on binding of the TRGDC-expressing cell to the TRGDC antibody. In one aspect, provided herein is a method of isolating a TRGDC-expressing cell comprising: contacting a TRGDC-expressing cell with a TRGDC antibody provided herein; and isolating the TRGDC-expressing cell based on binding of the TRGDC-expressing cell to the TRGDC antibody. In one aspect, provided herein is a method of separating a TRGDC-expressing cell comprising: contacting a TRGDC-expressing cell with a TRGDC antibody provided herein; and separating the TRGDC-expressing cell based on binding of the TRGDC-expressing cell to the TRGDC antibody. In one aspect, provided herein is a method of purifying a TRGDC-expressing cell comprising: contacting a TRGDC-expressing cell with a TRGDC antibody provided herein; and purifying the TRGDC-expressing cell based on binding of the TRGDC-expressing cell to the TRGDC antibody. In one aspect, provided herein is a method of sorting a TRGDC-expressing cell comprising: contacting a TRGDC-expressing cell with a TRGDC antibody provided herein; and sorting the TRGDC-expressing cell based on binding of the TRGDC-expressing cell to the TRGDC antibody. In one aspect, provided herein is a method of selecting a TRGDC-expressing cell comprising: contacting a TRGDC-expressing cell with a TRGDC antibody provided herein; and selecting the TRGDC-expressing cell based on binding of the TRGDC-expressing cell to the TRGDC antibody. In one aspect, provided herein is a method of capturing a TRGDC-expressing cell comprising: contacting a TRGDC-expressing cell with a TRGDC antibody provided herein; and capturing the TRGDC-expressing cell based on binding of the TRGDC-expressing cell to the TRGDC antibody. In one aspect, provided herein is a method of detecting a TRGDC-expressing cell comprising: contacting a TRGDC-expressing cell with a TRGDC antibody provided herein; and detecting the TRGDC-expressing cell based on binding of the TRGDC-expressing cell to the TRGDC antibody.

In certain embodiments of the methods, the TRGDC-expressing cell is a T cell. In some embodiments of the methods, the TRGDC-expressing cell is in a population of cells. In some embodiments of the methods, the TRGDC-expressing cell is in a population of lymphocytes. In some embodiments of the methods, the TRGDC-expressing cell is in a population of T cells. In some embodiments of the methods, the TRGDC-expressing cell is provided as a population of cells. In some embodiments of the methods, the TRGDC-expressing cell is provided as a population of lymphocytes. In some embodiments of the methods, the TRGDC-expressing cell is provided as a population of T cells. In some embodiments of the methods, the TRGDC-expressing cell is provided as a sample comprising a population of cells. In some embodiments of the methods, the TRGDC-expressing cell is provided as a sample comprising a population of lymphocytes. In some embodiments of the methods, the TRGDC-expressing cell is provided as a sample comprising a population of T cells. In some embodiments of the methods, the sample is a blood sample. In some embodiments of the methods, the sample is a tissue sample. In some embodiments of the methods, the sample is a tissue culture sample.

In some embodiments of the methods, the TRGDC antibody is a multispecific TRGDC antibody provided herein. In some embodiments of the methods, the TRGDC antibody is a bispecific TRGDC antibody provided herein. In some embodiments of the methods, the TRGDC antibody is a trispecific TRGDC antibody provided herein. In some embodiments of the methods, the TRGDC antibody is a quadraspecific TRGDC antibody provided herein. In certain embodiments, the TRGDC antibody specifically binds to TRGDC. In one embodiment, the multispecific TRGDC antibody comprises: (a) a first binding domain that binds TRGDC, and (b) a second binding domain that binds to a second target. In one embodiment, the multispecific TRGDC antibody comprises: (a) a first binding domain that binds TRGDC, and (b) a second binding domain that binds to a second target, and (c) a third binding domain that binds to a third target. In one embodiment, the multispecific TRGDC antibody comprises: (a) a first binding domain that binds TRGDC, and (b) a second binding domain that binds to a second target, (c) a third binding domain that binds to a third target, and (d) a fourth binding domain that binds to a fourth target. In one embodiment, the multispecific TRGDC antibody comprises: (a) a first binding domain that specifically binds TRGDC, and (b) a second binding domain that specifically binds to a second target. In one embodiment, the multispecific TRGDC antibody comprises: (a) a first binding domain that specifically binds TRGDC, and (b) a second binding domain that specifically binds to a second target, and (c) a third binding domain that specifically binds to a third target. In one embodiment, the multispecific TRGDC antibody comprises: (a) a first binding domain that specifically binds TRGDC, and (b) a second binding domain that specifically binds to a second target, (c) a third binding domain that specifically binds to a third target, and (d) a fourth binding domain that specifically binds to a fourth target.

In specific embodiments of the methods provided herein, the method uses multi-marker detection. In some embodiments, the multi-marker detection uses a multispecific TRGDC antibody provided herein. In some embodiments, the multi-marker detection uses a bispecific TRGDC antibody provided herein. In some embodiments, the multi-marker detection uses a trispecific TRGDC antibody provided herein. In some embodiments, the multi-marker detection uses a quadraspecific TRGDC antibody provided herein.

In certain embodiments of the methods provided herein, the methods are included as steps in a T cell manufacturing process. In certain embodiments, the cells are CAR-T cells. In certain embodiments of the methods provided herein, the methods are included as steps in a T cell modification process.

In certain embodiments of the methods provided herein, the methods are included as steps in a diagnostic method. In certain embodiments of the methods provided herein, the methods are included as steps in a method to quantify the TRGDC-expressing T cells.

In certain embodiments of the methods provided herein, the method further comprises expanding the enriched, isolated, separated, purified, sorted, selected, captured or detected TRGDC-expressing cells. In certain embodiments, the expanding is in vitro. In certain embodiments, the expanding is in vivo. In certain embodiments of the methods provided herein, the method further comprises growing the enriched, isolated, separated, purified, sorted, selected, captured or detected TRGDC-expressing cells. In certain embodiments, the growing is in vitro. In certain embodiments, the growing is in vivo. In certain embodiments of the methods provided herein, the method further comprises quantifying the enriched, isolated, separated, purified, sorted, selected, captured or detected TRGDC-expressing cells.

### Embodiments

This invention provides the following non-limiting embodiments.

In one set of embodiments, provided are:
A1. An antibody that binds T cell receptor (TCR) Vγ9 (TRGV9), comprising:
   (1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32;
   (2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66;
   (3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
   (4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134;
   (5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:168;
   (6) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:201; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:202;
   (7) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236;
   (8) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:269; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
   (9) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:304;
   (10) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:337; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:338;
   (11) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372;
   (12) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406;
   (13) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:440;
   (14) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474;
   (15) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508;
   (16) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542;
   (17) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576;
   (18) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:609; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:610;
   (19) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:644;
   (20) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678;
   (21) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:712;
   (22) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746;
   (23) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780;
   (24) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814;
   (25) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:847; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:848;
   (26) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:881; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:882;
   (27) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:915; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:916;
   (28) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:949; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:950;
   (29) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:983; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:984;
   (30) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1017; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1018;
   (31) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1051; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1052;
   (32) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1085; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1086;
   (33) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1119; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1120;
   (34) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1153; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1154;
   (35) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1187; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1188;
   (36) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1221; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1222;
   (37) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1255; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1256;
   (38) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1289; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1290;
   (39) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1323; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1324;
   (40) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1357; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1358;
   (41) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1391; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1392;
   (42) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1425; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1426;
   (43) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1459; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1460;
   (44) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1493; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1494;
   (45) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1527; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1528;
   (46) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1561; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1562;
   (47) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1595; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1596;
   (48) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1629; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1630;
   (49) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1663; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1664;
   (50) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1697; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1698;
   (51) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1731; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1732;
   (52) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1765; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1766;
   (53) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1799; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1800;
   (54) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1833; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1834;
   (55) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1867; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1868;
   (56) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1901; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1902;
   (57) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1935; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1936;
   (58) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1969; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1970; or
   (59) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2003; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2004.
A2. The antibody of embodiment A1, wherein the VH CDR1, VH CDR2, VH CDR3, VL CDR1, VL CDR2, and VL CDR3 amino acid sequences are according to the Kabat numbering system.
A3. The antibody of embodiment A1, wherein the VH CDR1, VH CDR2, VH CDR3, VL CDR1, VL CDR2, and VL CDR3 amino acid sequences are according to the Chothia numbering system.
A4. The antibody of embodiment A1, wherein the VH CDR1, VH CDR2, VH CDR3, VL CDR1, VL CDR2, and VL CDR3 amino acid sequences are according to the AbM numbering system.
A5. The antibody of embodiment A1, wherein the VH CDR1, VH CDR2, VH CDR3, VL CDR1, VL CDR2, and VL CDR3 amino acid sequences are according to the Contact numbering system.
A6. The antibody of embodiment A1, wherein the VH CDR1, VH CDR2, VH CDR3, VL CDR1, VL CDR2, and VL CDR3 amino acid sequences are according to the IMGT numbering system.
A7. The antibody of any one of embodiments A1 to A6, wherein the antibody is a humanized antibody.
A8. The antibody of any one of embodiments A1 to A7, wherein the antibody is an IgG antibody.
A9. The antibody of embodiment A8, wherein the IgG antibody is an IgG1, IgG2, IgG3, or IgG4 antibody.
A10. The antibody of any one of embodiments A1 to A9, wherein the antibody comprises a kappa light chain.
A11. The antibody of any one of embodiments A1 to A9, wherein the antibody comprises a lambda light chain.
A12. The antibody of any one of embodiments A1 to A11, wherein the antibody is a monoclonal antibody.
A13. The antibody of any one of embodiments A1 to A12, wherein the antibody binds a TRGV9 antigen.
A14. The antibody of any one of embodiments A1 to A12, wherein antibody binds a TRGV9 epitope.
A15. The antibody of any one of embodiments A1 to A14, wherein the antibody specifically binds to TRGV9.
A16. The antibody of any one of embodiments A1 to A15, wherein the VH CDR1, VH CDR2, VH CDR3, VL CDR1, VL CDR2 and VL CDR3 form a binding site for an antigen of the TRGV9.
A17. The antibody of any one of embodiments A1 to A15, wherein the VH CDR1, VH CDR2, VH CDR3, VL CDR1, VL CDR2 and VL CDR3 form a binding site for an epitope of the TRGV9.
A18. The antibody of any one of embodiments A1 to A17, wherein the TRGV9 is present on the surface of a T cell.
A19. The antibody of any one of embodiments A1 to A18, wherein the antibody is multivalent.
A20. The antibody of embodiment A19, wherein the antibody is capable of binding at least three antigens.
A21. The antibody of embodiment A19, wherein the antibody is capable of binding at least five antigens.
A22. The antibody of any one of embodiments A1 to A21, wherein the antibody is a multispecific antibody.
A23. The antibody of embodiment A22, wherein the antibody is a bispecific antibody.
A24. A nucleic acid sequence encoding the antibody of any one of embodiments A1 to A23.
A25. A vector comprising the nucleic acid sequence of embodiment A24.
A26. A host cell comprising the vector of embodiment A25.
A27. A kit comprising the vector of embodiment A25 and packaging for the same.
A28. A kit comprising the antibody of any one of embodiments A1 to A23 and packaging for the same.
A29. A pharmaceutical composition comprising the antibody of any one of embodiments A1 to A23, and a pharmaceutically acceptable carrier.
A30. A method of producing the pharmaceutical composition of embodiment A29, comprising combining the antibody with a pharmaceutically acceptable carrier to obtain the pharmaceutical composition.
A31. A method of activating a T cell expressing TRGV9, comprising contacting the T cell with the antibody of any one of embodiments A1 to A23.
A32. The method of embodiment A31, wherein the contacting results in an increase in CD69, CD25, and/or Granzyme B expression, as compared to a control T cell expressing TRGV9.
B1. An antibody that binds TCR V62 (TRDV2), comprising:
   (1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2037; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2038;
   (2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2071; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2072;
   (3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2105; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2106;
   (4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2139; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2140;
   (5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2173; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2174;
   (6) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2207; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2208;
   (7) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2241; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2242;
   (8) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2275; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2276;
   (9) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2309; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2310;
   (10) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2343; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2344;
   (11) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2377; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2378;
   (12) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2411; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2412;
   (13) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2445; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2446;
   (14) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2479; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2480;
   (15) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2513; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2514;
   (16) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2547; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2548;
   (17) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2581; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2582;
   (18) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2615; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2616;
   (19) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2649; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2650;
   (20) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2683; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2684;
   (21) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2717; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2718;
   (22) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2751; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2752;
   (23) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2785; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2786;
   (24) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2819; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2820;
   (25) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2853; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2854;
   (26) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2887; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2888;
   (27) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2921; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2922;
   (28) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2955; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2956;
   (29) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2989; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2990;
   (30) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3023; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3024;
   (31) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3057; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3058;
   (32) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3091; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3092;
   (33) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3125; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3126;
   (34) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3159; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3160;
   (35) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3193; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3194;
   (36) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3227; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3228;
   (37) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3261; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3262;
   (38) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3295; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3296;
   (39) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3329; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3330;
   (40) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3363; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3364;
   (41) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3397; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3398;
   (42) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3431; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3432;
   (43) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3465; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3466;
   (44) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3499; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3500;
   (45) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3533; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3534;
   (46) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3567; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3568;
   (47) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3601; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3602;
   (48) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3635; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3636;
   (49) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3669; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3670;
   (50) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3703; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3704;
   (51) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3737; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3738;
   (52) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3771; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3772;
   (53) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3805; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3806;
   (54) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3839; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3840;
   (55) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3873; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3874;
   (56) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3907; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3908;
   (57) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3941; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3942;
   (58) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:3975; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3976;
   (59) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4009; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4010;
   (60) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4043; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4044;
   (61) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4077; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4078; or
   (62) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4111; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4112.
B2. The antibody of embodiment B1, wherein the VH CDR1, VH CDR2, VH CDR3, VL CDR1, VL CDR2, and VL CDR3 amino acid sequences are according to the Kabat numbering system.
B3. The antibody of embodiment B1, wherein the VH CDR1, VH CDR2, VH CDR3, VL CDR1, VL CDR2, and VL CDR3 amino acid sequences are according to the Chothia numbering system.
B4. The antibody of embodiment B1, wherein the VH CDR1, VH CDR2, VH CDR3, VL CDR1, VL CDR2, and VL CDR3 amino acid sequences are according to the AbM numbering system.
B5. The antibody of embodiment B1, wherein the VH CDR1, VH CDR2, VH CDR3, VL CDR1, VL CDR2, and VL CDR3 amino acid sequences are according to the Contact numbering system.
B6. The antibody of embodiment B1, wherein the VH CDR1, VH CDR2, VH CDR3, VL CDR1, VL CDR2, and VL CDR3 amino acid sequences are according to the IMGT numbering system.
B7. The antibody of any one of embodiments B1 to B6, wherein the antibody is a humanized antibody.
B8. The antibody of any one of embodiments B1 to B7, wherein the antibody is an IgG antibody.
B9. The antibody of embodiment B8, wherein the IgG antibody is an IgG1, IgG2, IgG3, or IgG4 antibody.
B10. The antibody of any one of embodiments B1 to B9, wherein the antibody comprises a kappa light chain.
B11. The antibody of any one of embodiments B1 to B9, wherein the antibody comprises a lambda light chain.
B12. The antibody of any one of embodiments B1 to B11, wherein the antibody is a monoclonal antibody.
B13. The antibody of any one of embodiments B1 to B12, wherein the antibody binds a TRDV2 antigen.
B14. The antibody of any one of embodiments B1 to B12, wherein antibody binds a TRDV2 epitope.
B15. The antibody of any one of embodiments B1 to B14, wherein the antibody specifically binds to TRDV2.
B16. The antibody of any one of embodiments B1 to B15, wherein the VH CDR1, VH CDR2, VH CDR3, VL CDR1, VL CDR2 and VL CDR3 form a binding site for an antigen of the TRDV2.
B17. The antibody of any one of embodiments B1 to B15, wherein the VH CDR1, VH CDR2, VH CDR3, VL CDR1, VL CDR2 and VL CDR3 form a binding site for an epitope of the TRDV2.
B18. The antibody of any one of embodiments B1 to B17, wherein the TRDV2 is present on the surface of a T cell.
B19. The antibody of any one of embodiments B1 to B18, wherein the antibody is multivalent.
B20. The antibody of embodiment B19, wherein the antibody is capable of binding at least three antigens.
B21. The antibody of embodiment B19, wherein the antibody is capable of binding at least five antigens.
B22. The antibody of any one of embodiments B1 to B21, wherein the antibody is a multispecific antibody.
B23. The antibody of embodiment B22, wherein the antibody is a bispecific antibody.
B24. A nucleic acid sequence encoding the antibody of any one of embodiments B1 to B23.
B25. A vector comprising the nucleic acid sequence of embodiment B24.
B26. A host cell comprising the vector of embodiment B25.
B27. A kit comprising the vector of embodiment B25 and packaging for the same.
B28. A kit comprising the antibody of any one of embodiments B1 to B23 and packaging for the same.
B29. A pharmaceutical composition comprising the antibody of any one of embodiments B1 to B23, and a pharmaceutically acceptable carrier.
B30. A method of producing the pharmaceutical composition of embodiment B29, comprising combining the antibody with a pharmaceutically acceptable carrier to obtain the pharmaceutical composition.
B31. A method of activating a T cell expressing TRDV2, comprising contacting the T cell with the antibody of any one of embodiments B1 to B23.
B32. The method of embodiment B31, wherein the contacting results in an increase in CD69, CD25, and/or Granzyme B expression, as compared to a control T cell expressing TRDV2.
C1. An antibody that binds TCR gamma delta constant region (TRGDC), comprising:
   (1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4145; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4146;
   (2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4179; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4180;
   (3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4213; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4214;
   (4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4247; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4248;
   (5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4281; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4282;
   (6) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4315; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4316;
   (7) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4349; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4350;
   (8) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4383; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4384;
   (9) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4417; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4418;
   (10) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4451; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4452;
   (11) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4485; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4486;
   (12) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4519; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4520;
   (13) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4553; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4554;
   (14) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4587; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4588;
   (15) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4621; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4622; or
   (16) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:4655; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:4656.
C2. The antibody of embodiment C1, wherein the VH CDR1, VH CDR2, VH CDR3, VL CDR1, VL CDR2, and VL CDR3 amino acid sequences are according to the Kabat numbering system.
C3. The antibody of embodiment C1, wherein the VH CDR1, VH CDR2, VH CDR3, VL CDR1, VL CDR2, and VL CDR3 amino acid sequences are according to the Chothia numbering system.
C4. The antibody of embodiment C1, wherein the VH CDR1, VH CDR2, VH CDR3, VL CDR1, VL CDR2, and VL CDR3 amino acid sequences are according to the AbM numbering system.
C5. The antibody of embodiment C1, wherein the VH CDR1, VH CDR2, VH CDR3, VL CDR1, VL CDR2, and VL CDR3 amino acid sequences are according to the Contact numbering system.
C6. The antibody of embodiment C1, wherein the VH CDR1, VH CDR2, VH CDR3, VL CDR1, VL CDR2, and VL CDR3 amino acid sequences are according to the IMGT numbering system.
C7. The antibody of any one of embodiments C1 to C6, wherein the antibody is a humanized antibody.
C8. The antibody of any one of embodiments C1 to C7, wherein the antibody is an IgG antibody.
C9. The antibody of embodiment C8, wherein the IgG antibody is an IgG1, IgG2, IgG3, or IgG4 antibody.
C10. The antibody of any one of embodiments C1 to C9, wherein the antibody comprises a kappa light chain.
C11. The antibody of any one of embodiments C1 to C9, wherein the antibody comprises a lambda light chain.
C12. The antibody of any one of embodiments C1 to C11, wherein the antibody is a monoclonal antibody.
C13. The antibody of any one of embodiments C1 to C12, wherein the antibody binds a TRGDC antigen.
C14. The antibody of any one of embodiments C1 to C12, wherein antibody binds a TRGDC epitope.
C15. The antibody of any one of embodiments C1 to C14, wherein the antibody specifically binds to TRGDC.
C16. The antibody of any one of embodiments C1 to C15, wherein the VH CDR1, VH CDR2, VH CDR3, VL CDR1, VL CDR2 and VL CDR3 form a binding site for an antigen of the TRGDC.
C17. The antibody of any one of embodiments C1 to C15, wherein the VH CDR1, VH CDR2, VH CDR3, VL CDR1, VL CDR2 and VL CDR3 form a binding site for an epitope of the TRGDC.
C18. The antibody of any one of embodiments C1 to C17, wherein the TRGDC is present on the surface of a T cell.
C19. The antibody of any one of embodiments C1 to C18, wherein the antibody is multivalent.
C20. The antibody of embodiment C19, wherein the antibody is capable of binding at least three antigens.
C21. The antibody of embodiment C19, wherein the antibody is capable of binding at least five antigens.
C22. The antibody of any one of embodiments C1 to C21, wherein the antibody is a multispecific antibody.
C23. The antibody of embodiment C22, wherein the antibody is a bispecific antibody.
C24. A nucleic acid sequence encoding the antibody of any one of embodiments C1 to C23.
C25. A vector comprising the nucleic acid sequence of embodiment C24.
C26. A host cell comprising the vector of embodiment C25.
C27. A kit comprising the vector of embodiment C25 and packaging for the same.
C28. A kit comprising the antibody of any one of embodiments C1 to C23 and packaging for the same.
C29. A pharmaceutical composition comprising the antibody of any one of embodiments C1 to C23, and a pharmaceutically acceptable carrier.
C30. A method of producing the pharmaceutical composition of embodiment C29, comprising combining the antibody with a pharmaceutically acceptable carrier to obtain the pharmaceutical composition.
C31. A method of activating a T cell expressing TRGDC, comprising contacting the T cell with the antibody of any one of embodiments C1 to C23.
C32. The method of embodiment C31, wherein the contacting results in an increase in CD69, CD25, and/or Granzyme B expression, as compared to a control T cell expressing TRGDC.

Provided in the Examples herein are exemplary antibodies that bind to TRGV9. Also provided in the Examples herein are exemplary antibodies that bind to TRDV2. Also provided in the Examples herein are exemplary antibodies that bind to TRGDC. Exemplary binding agents that bind to TRGV9 are provided herein, for example in the Examples, as well as Tables 2-7. Exemplary binding agents that bind to TRDV2 are provided herein, for example in the Examples, as well as Tables 2-7. Exemplary binding agents that bind to TRGDC are provided herein, for example in the Examples, as well as Tables 2-7.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### EXAMPLES

### Example 1: Production of Antibodies that Bind γδ T Cells

1.1: Production of Mabs that Bind γδ T Cell Antigens

Antigens or portions of antigens specific for γδ T cells are used to immunize an animal (e.g., a mouse or a rabbit). To generate the γδ T cell monoclonal antibodies, peripheral blood mononuclear cells are isolated from the whole blood of the immunized animal, and antigen specific B cells are grown. B cells secreting reactive antibodies for the γδ T cell antigens are identified by an antigen-binding ELISA screening of the B cell culture supernatants. High binding ELISA plates are coated with the γδ T cell antigen overnight. The ELISA plates are blocked, and diluted B cell culture supernatants are added to the plates. The plates are incubated at room temperature and following incubation, a secondary antibody specific for recognizing the γδ T cell antigen antibody is added to the plate to determine if the γδ T cell antigen antibody bound the γδ T cell antigen. Binding of the antibody is determined by reaction of a substrate on the secondary antibody.

After the identification of monoclonal antibodies that are capable of binding γδ T cell antigens, the variable regions of the heavy and light chains of the γδ T cell antibody are sequenced. γδ T cell antibodies that bind to TRGV9, TRDV2 and TRGDC are produced. Constructs are created for the expression of the heavy and light chain of the γδ T cell antibody. The constructs are transfected into a host cell to express the heavy and light chains, and the γδ T cell antibody is isolated from the supernatant.

### 1.2: Generation of Trgv9 Antibodies, Trdv2 Antibodies, and Trgdc Antibodies

Immunogen. A recombinant human TCR Vγ9×Vδ2 fused to a human Fc was used as an immunogen, and the sequence is listed in Table 1.

TABLE 1
Amino acid sequence of recombinant human TCR Vγ9 × Vδ2
heterodimeric protein fused to human Fc

| | Protein | | SEQ ID |
|---|---|---|---|
| Name | ID | Sequence | NO: |
| Recombinant | Vg9 | MAWVWTLLFLMAAAQSIQAAGHLEQPQISSTKTLSKTARLECVV | 4659 |
| human [TCR | chain | SGITISATSVYWYRERPGEVIQFLVSISYDGTVRKESGIPSGKF | |
| Vg9 × Vd2]- | | EVDRIPETSTSTLTIHNVEKQDIATYYCALWEAQQELGKKIKVF | |
| hFc | | GPGTKLIITDKQLDADVSPKPTIFLPSIAETKLQKAGTYLCLLE | |
| | | KFFPDVIKIHWEEKKSNTILGSQEGNTMKTNDTYMKFSWLTVPE | |
| | | KSLDKEHRCIVRHENNKNGVDQEIIFPPIKTDVITMDPKDNEPK | |
| | | SCDKTHTCPPCPAPELLGGPSVFLFPPKPKDTLMISRTPEVTCV | |
| | | VVDVSHEDPEVKFNWYVDGVEVHNAKTKPREEQYNSTYRVVSVL | |
| | | TVLHQDWLNGKEYKCKVSNKALPAPIEKTISKAKGQPREPQVYV | |
| | | YPPSREEMTKNQVSLTCLVKGFYPSDIAVEWESNGQPENNYKTT | |
| | | PPVLDSDGSFALVSKLTVDKSRWQQGNVFSCSVMHEALHNHYTQ | |
| | | KSLSLSPGK | |
| | Vd2 | MAWVWTLLFLMAAAQSIQAAIELVPEHQTVPVSIGVPATLRCSM | 4660 |
| | chain | KGEAIGNYYINWYRKTQGNTMTFIYREKDIYGPGFKDNFQGDID | |
| | | IAKNLAVLKILAPSERDEGSYYCACDTLGMGGEYTDKLIFGKGT | |
| | | RVTVEPRSQPHTKPSVFVMKNGTNVACLVKEFYPKDIRINLVSS | |
| | | KKITEFDPAIVISPSGKYNAVKLGKYEDSNSVTCSVQHDNKTVH | |
| | | STDFEVKTDSTDHVKPKETENTKQPSKSEPKSCDKTHTCPPCPA | |
| | | PELLGGPSVFLFPPKPKDTLMISRTPEVTCVVVDVSHEDPEVKF | |
| | | NWYVDGVEVHNAKTKPREEQYNSTYRVVSVLTVLHQDWLNGKEY | |
| | | KCKVSNKALPAPIEKTISKAKGQPREPQVYVLPPSREEMTKNQV | |
| | | SLLCLVKGFYPSDIAVEWESNGQPENNYLTWPPVLDSDGSFFLY | |
| | | SKLTVDKSRWQQGNVFSCSVMHEALHNHYTQKSLSLSPGK | |

Protein Production of the Immunogen. Expression plasmids encoding the immunogen (see Table 1) were transfected into CHO cell at a DNA ratio of 1:1. Total amount of DNA for a 750 mL expression scale was 750 μg. Final expression volume was 1 L after two feedings and enhancer additions. Using an ÄKTAPRIME plus instrument (GE Healthcare Life Sciences), supernatant (1 L) after 7 days was applied with a flow-rate of 5 mL/min to a MABSELECT SURE (GE Life Sciences) with a column volume (CV) of 10 mL pre-equilibrated with phosphate buffered saline (PBS), pH 6.8. Non-specific proteins binding to the column material was washed off with PBS supplemented with 500 mM NaCl, pH 6.8 (5 CV). The Fc-containing immunogen was eluted stepwise with 40 mM sodium acetate pH 5.0 (5 CV), pH 4.5 (5 CV), pH 4.0 (10 CV), pH 3.5 (5 CV), and pH 3.0 (5 CV). Fractions were pooled, and applied (5 mL) at a flow-rate of 0.2 mL/min on to a HiLoad 16/600 SUPERDEX (GE Healthcare) column pre-equilibrated with PBS (pH 6.8). Target protein was eluted, pooled, and analyzed by SDS-PAGE, analytic SEC, and intact mass by mass spectrometry. Purity was estimated to >99%.

Antibodies were generated using ALIVAMAB transgenic mice technology (Ablexis). ALIVAMAB mice were immunized with recombinant human Vγ9/Vδ2 TCR protein. Lymphocytes were extracted from secondary lymphoid organs and either fused with FO mouse myeloma cell line for hybridoma generation or subjected to single cell sorting via FACS. Hybridoma supernatants were screened by MSD electrochemiluminescence or by FACS for binding to γδ T cells. Confirmed cell binders were light chain isotyped via ELISA. Single cell sorting supernatants were screened by MSD electrochemiluminescence for binding to recombinant human Vγ9/Vδ2 protein. Several hits with the desired binding profile were selected and sequenced, as provided below.

The VH and VL sequences of certain TRGV9, TRDV2, and TRGDC antibodies are provided in Table 2. The CDRs sequences of certain TRGV9, TRDV2, and TRGDC antibodies are provided in Table 3 (Kabat), Table 4 (Chothia), Table 5 (AbM), Table 6 (Contact), and Table 7 (IMGT).

TABLE 2
VH and VL Amino Acid Sequences

| | Protein | HC | LC | VH AA | VL AA | | |
|---|---|---|---|---|---|---|---|
| # | Name | Isotype | Isotype | sequence | sequence | Heavy Chain AA sequence | Light Chain AA sequence |
| 1 | VG9B54 | IgG1 | Kappa | EVQLVESGGGL | EIVMTQSPGT | EVQLVESGGGLVKPGGSLRLSCAASGFAFSSYSLTWVR | EIVMTQSPGTLSLSPGERA |
| | | | | VKPGGSLRLSC | LSLSPGERAT | QAPGKGLEWVSSISSSSSYIFYADSVKGRFTISRDNAK | TLSCRASQSVSSSYLAWFQ |
| | | | | AASGFAFSSYS | LSCRASQSVS | NSLYLQMNSLRAEDTAVYYCATDGELGPFDYWGQGTTV | QKPGQAPRRLIYGASSRAT |
| | | | | LTWVRQAPGKG | SSYLAWFQQK | TVSSASTKGPSVFPLAPSSKSTSGGTAALGCLVKDYFP | GIPDRFSGSGSGTDFTLTI |
| | | | | LEWVSSISSSS | PGQAPRRLIY | EPVTVSWNSGALTSGVHTFPAVLQSSGLYSLSSVVTVP | SRLEPEDFAVYYCQQYGRS |
| | | | | SYIFYADSVKG | GASSRATGIP | SSSLGTQTYICNVNHKPSNTKVDKKVEPKSCDKTHTCP | PLTFGGGTKVEIKRTVAAP |
| | | | | RFTISRDNAKN | DRFSGSGSGT | PCPAPELLGGPSVFLFPPKPKDTLMISRTPEVTCVVVD | SVFIFPPSDEQLKSGTASV |
| | | | | SLYLQMNSLRA | DFTLTISRLE | VSHEDPEVKFNWYVDGVEVHNAKTKPREEQYNSTYRVV | VCLLNNFYPREAKVQWKVD |
| | | | | EDTAVYYCATD | PEDFAVYYCQ | SVLTVLHQDWLNGKEYKCKVSNKALPAPIEKTISKAKG | NALQSGNSQESVTEQDSKD |
| | | | | GELGPFDYWGQ | QYGRSPLTFG | QPREPQVYTLPPSREEMTKNQVSLTCLVKGFYPSDIAV | STYSLSSTLTLSKADYEKH |
| | | | | GTTVTVSS | GGTKVEIK | EWESNGQPENNYKTTPPVLDSDGSFFLYSKLTVDKSRW | KVYACEVTHQGLSSPVTKS |
| | | | | | | QQGNVFSCSVMHEALHNHYTQKSLSLSPGK | FNRGEC |
| | | | | 31 | 32 | 33 | 34 |
| 2 | VG9B121 | IgG1 | Kappa | EVQLVESGGGL | DIQMTQSPGT | EVQLVESGGGLVKPGGSLRLSCVASGFTFSDYAMDWVR | DIQMTQSPGTLSSSPGERA |
| | | | | VKPGGSLRLSC | LSSSPGERAT | QAPGKGLDWISSISSTSNYIFYADSVKGRFTISRDNAK | TLSCRASQSVSSSYFAWYQ |
| | | | | VASGFTFSDYA | LSCRASQSVS | NSLYLQMNSLRVEDSAVYYCANSYNWNYGGAFDIWGQG | QKPGRAPRLLIYGASSRAT |
| | | | | MDWVRQAPGKG | SSYFAWYQQK | TMVTVSSASTKGPSVFPLAPSSKSTSGGTAALGCLVKD | GIPDRFSGSGSGTDFTLTI |
| | | | | LDWISSISSTS | PGRAPRLLIY | YFPEPVTVSWNSGALTSGVHTFPAVLQSSGLYSLSSVV | SRLEPEDFAVYFCQQYGRS |
| | | | | NYIFYADSVKG | GASSRATGIP | TVPSSSLGTQTYICNVNHKPSNTKVDKKVEPKSCDKTH | PLTFGGGTKVEIKRTVAAP |
| | | | | RFTISRDNAKN | DRFSGSGSGT | TCPPCPAPELLGGPSVFLFPPKPKDTLMISRTPEVTCV | SVFIFPPSDEQLKSGTASV |
| | | | | SLYLQMNSLRV | DFTLTISRLE | VVDVSHEDPEVKFNWYVDGVEVHNAKTKPREEQYNSTY | VCLLNNFYPREAKVQWKVD |
| | | | | EDSAVYYCANS | PEDFAVYFCQ | RVVSVLTVLHQDWLNGKEYKCKVSNKALPAPIEKTISK | NALQSGNSQESVTEQDSKD |
| | | | | YNWNYGGAFDI | QYGRSPLTFG | AKGQPREPQVYTLPPSREEMTKNQVSLTCLVKGFYPSD | STYSLSSTLTLSKADYEKH |
| | | | | WGQGTMVTVSS | GGTKVEIK | IAVEWESNGQPENNYKTTPPVLDSDGSFFLYSKLTVDK | KVYACEVTHQGLSSPVTKS |
| | | | | | | SRWQQGNVFSCSVMHEALHNHYTQKSLSLSPGK | FNRGEC |
| | | | | 65 | 66 | 67 | 68 |
| 3 | VG9B429 | IgG1 | Kappa | QVQLVQSGGGL | DIQMTQSPSS | QVQLVQSGGGLVKPGGSLRLSCSASGFTFSNYDMNWVR | DIQMTQSPSSVSASVGDRV |
| | | | | VKPGGSLRLSC | VSASVGDRVT | QAPGKGLEWVSSISSSSSYIYYADSVKGRFTISRDNAK | TITCRASQGINSWLAWYQQ |
| | | | | SASGFTFSNYD | ITCRASQGIN | NSLYLQMNSLRAEDTAVYHCARDVGVTTDYYYYGMDVW | KPGNAPKLLIYAASSLQSG |
| | | | | MNWVRQAPGKG | SWLAWYQQKP | GQGTMVTVSSASTKGPSVFPLAPSSKSTSGGTAALGCL | VPSRFSGIGSGTDFIFTIS |
| | | | | LEWVSSISSSS | GNAPKLLIYA | VKDYFPEPVTVSWNSGALTSGVHTFPAVLQSSGLYSLS | SLQPEDFASYYCQQANSFP |
| | | | | SYIYYADSVKG | ASSLQSGVPS | SVVTVPSSSLGTQTYICNVNHKPSNTKVDKKVEPKSCD | WTFGQGTRLEIKRTVAAPS |
| | | | | RFTISRDNAKN | RFSGIGSGTD | KTHTCPPCPAPELLGGPSVFLFPPKPKDTLMISRTPEV | VFIFPPSDEQLKSGTASVV |
| | | | | SLYLQMNSLRA | FIFTISSLQP | TCVVVDVSHEDPEVKFNWYVDGVEVHNAKTKPREEQYN | CLLNNFYPREAKVQWKVDN |
| | | | | EDTAVYHCARD | EDFASYYCQQ | STYRVVSVLTVLHQDWLNGKEYKCKVSNKALPAPIEKT | ALQSGNSQESVTEQDSKDS |
| | | | | VGVTTDYYYYG | ANSFPWTFGQ | ISKAKGQPREPQVYTLPPSREEMTKNQVSLTCLVKGFY | TYSLSSTLTLSKADYEKHK |
| | | | | MDVWGQGTMVT | GTRLEIK | PSDIAVEWESNGQPENNYKTTPPVLDSDGSFFLYSKLT | VYACEVTHQGLSSPVTKSF |
| | | | | VSS | | VDKSRWQQGNVFSCSVMHEALHNHYTQKSLSLSPGK | NRGEC |
| | | | | 99 | 100 | 101 | 102 |
| 4 | VG9B370 | IgG1 | Kappa | EVQLVESGGGL | EIVLTQSPGT | EVQLVESGGGLVKPGGSLRLSCAASRFTLSSYDMNWVR | EIVLTQSPGTLSLSPGERA |
| | | | | VKPGGSLRLSC | LSLSPGERAT | QAPGKGLEWVSSISSSSSYIYYADSVKGRFTISRDNAK | TLSCRASQSVSSSYLAWYQ |
| | | | | AASRFTLSSYD | LSCRASQSVS | NSLYLQMNSLRAEDTAVYYCARDRGVGGTDYYYYGLDV | QKPGQAPRLLIYGASSRAT |
| | | | | MNWVRQAPGKG | SSYLAWYQQK | WGQGTTVTVSSASTKGPSVFPLAPSSKSTSGGTAALGC | GIPDRFSGSGSGTDFTLTI |
| | | | | LEWVSSISSSS | PGQAPRLLIY | LVKDYFPEPVTVSWNSGALTSGVHTFPAVLQSSGLYSL | SRLEPEDFAVYYCQQYGSS |
| | | | | SYIYYADSVKG | GASSRATGIP | SSVVTVPSSSLGTQTYICNVNHKPSNTKVDKKVEPKSC | PPYTFGQGTKVEIKRTVAA |
| | | | | RFTISRDNAKN | DRFSGSGSGT | DKTHTCPPCPAPELLGGPSVFLFPPKPKDTLMISRTPE | PSVFIFPPSDEQLKSGTAS |
| | | | | SLYLQMNSLRA | DFTLTISRLE | VTCVVVDVSHEDPEVKFNWYVDGVEVHNAKTKPREEQY | VVCLLNNFYPREAKVQWKV |
| | | | | EDTAVYYCARD | PEDFAVYYCQ | NSTYRVVSVLTVLHQDWLNGKEYKCKVSNKALPAPIEK | DNALQSGNSQESVTEQDSK |
| | | | | RGVGGTDYYYY | QYGSSPPYTF | TISKAKGQPREPQVYTLPPSREEMTKNQVSLTCLVKGF | DSTYSLSSTLTLSKADYEK |
| | | | | GLDVWGQGTTV | GQGTKVEIK | YPSDIAVEWESNGQPENNYKTTPPVLDSDGSFFLYSKL | HKVYACEVTHQGLSSPVTK |
| | | | | TVSS | | TVDKSRWQQGNVFSCSVMHEALHNHYTQKSLSLSPGK | SFNRGEC |
| | | | | 133 | 134 | 135 | 136 |
| 5 | VG9B80 | IgG1 | Kappa | EVQLVQSGGGL | EIVLTQSPGT | EVQLVQSGGGLVKSGGSLRLSCAASGFTFSTYSMNWVR | EIVLTQSPGTLSLSAGERA |
| | | | | VKSGGSLRLSC | LSLSAGERAT | QAPGKGLEWVSSISSSSSYIFYADSVKGRIIISRDNAK | TLSCRASQSISSSYLAWYQ |
| | | | | AASGFTFSTYS | LSCRASQSIS | NSLYLQMNSVRADDTAVYYCAKDGELGVFDYWGQGTLV | QKPGQAPRVLIYGPSGRAT |
| | | | | MNWVRQAPGKG | SSYLAWYQQK | TVSSASTKGPSVFPLAPSSKSTSGGTAALGCLVKDYFP | GIPDRFRGSGSGTDFTLTI |
| | | | | LEWVSSISSSS | PGQAPRVLIY | EPVTVSWNSGALTSGVHTFPAVLQSSGLYSLSSVVTVP | SRLEPEDFAVYYCQQFGRS |
| | | | | SYIFYADSVKG | GPSGRATGIP | SSSLGTQTYICNVNHKPSNTKVDKKVEPKSCDKTHTCP | PLTFGGGTKVEIKRTVAAP |
| | | | | RIIISRDNAKN | DRFRGSGSGT | PCPAPELLGGPSVFLFPPKPKDTLMISRTPEVTCVVVD | SVFIFPPSDEQLKSGTASV |
| | | | | SLYLQMNSVRA | DFTLTISRLE | VSHEDPEVKFNWYVDGVEVHNAKTKPREEQYNSTYRVV | VCLLNNFYPREAKVQWKVD |
| | | | | DDTAVYYCAKD | PEDFAVYYCQ | SVLTVLHQDWLNGKEYKCKVSNKALPAPIEKTISKAKG | NALQSGNSQESVTEQDSKD |
| | | | | GELGVFDYWGQ | QFGRSPLTFG | QPREPQVYTLPPSREEMTKNQVSLTCLVKGFYPSDIAV | STYSLSSTLTLSKADYEKH |
| | | | | GTLVTVSS | GGTKVEIK | EWESNGQPENNYKTTPPVLDSDGSFFLYSKLTVDKSRW | KVYACEVTHQGLSSPVTKS |
| | | | | | | QQGNVFSCSVMHEALHNHYTQKSLSLSPGK | FNRGEC |
| | | | | 167 | 168 | 169 | 170 |
| 6 | VG9B414 | IgG1 | Kappa | EVQLVESGGGL | EIVMTQSPSS | EVQLVESGGGLVKPGGSLRLSCSASGFTFSNYDMNWVR | EIVMTQSPSSVSASVGDRV |
| | | | | VKPGGSLRLSC | VSASVGDRVT | QAPGKGLEWVSSISSSSSYIYYADSVKGRFTISRDNAK | TITCRASQGINSWLAWYQQ |
| | | | | SASGFTFSNYD | ITCRASQGIN | NSLYLQMNSLRAEDTAVYHCARDVGVTTDYYYYGMDVW | KPGNAPKLLIYAASSLQSG |
| | | | | MNWVRQAPGKG | SWLAWYQQKP | GQGTMVTVSSASTKGPSVFPLAPSSKSTSGGTAALGCL | VPSRFSGIGSGTDFIFTIS |
| | | | | LEWVSSISSSS | GNAPKLLIYA | VKDYFPEPVTVSWNSGALTSGVHTFPAVLQSSGLYSLS | SLQPEDFASYYCQQANSFP |
| | | | | SYIYYADSVKG | ASSLQSGVPS | SVVTVPSSSLGTQTYICNVNHKPSNTKVDKKVEPKSCD | WTFGQGTRLEIKRTVAAPS |
| | | | | RFTISRDNAKN | RFSGIGSGTD | KTHTCPPCPAPELLGGPSVFLFPPKPKDTLMISRTPEV | VFIFPPSDEQLKSGTASVV |
| | | | | SLYLQMNSLRA | FIFTISSLQP | TCVVVDVSHEDPEVKFNWYVDGVEVHNAKTKPREEQYN | CLLNNFYPREAKVQWKVDN |
| | | | | EDTAVYHCARD | EDFASYYCQQ | STYRVVSVLTVLHQDWLNGKEYKCKVSNKALPAPIEKT | ALQSGNSQESVTEQDSKDS |
| | | | | VGVTTDYYYYG | ANSFPWTFGQ | ISKAKGQPREPQVYTLPPSREEMTKNQVSLTCLVKGFY | TYSLSSTLTLSKADYEKHK |
| | | | | MDVWGQGTMVT | GTRLEIK | PSDIAVEWESNGQPENNYKTTPPVLDSDGSFFLYSKLT | VYACEVTHQGLSSPVTKSF |
| | | | | VSS | | VDKSRWQQGNVFSCSVMHEALHNHYTQKSLSLSPGK | NRGEC |
| | | | | 201 | 202 | 203 | 204 |
| 7 | VG9B195 | IgG1 | Lambda | EVQLVESGGGL | SYELMQPPSV | EVQLVESGGGLVKPGGSLRLSCAASGFTFSPYTMNWVR | SYELMQPPSVSVSPGQTAS |
| | | | | VKPGGSLRLSC | SVSPGQTASI | QAPGKGLEWVSSISSSSSYMYYADSVKGRFTISRDNAK | ITCSGDKLGDKYACWYQQK |
| | | | | AASGFTFSPYT | TCSGDKLGDK | NSLYLQMNSLRAEDTAVYYCARDGDLVGPTYYFDYWGQ | PGQSPVLVIYQHNKRPSGI |
| | | | | MNWVRQAPGKG | YACWYQQKPG | GTLVTVSSASTKGPSVFPLAPSSKSTSGGTAALGCLVK | PERFSGSNSGKTATLTISG |
| | | | | LEWVSSISSSS | QSPVLVIYQH | DYFPEPVTVSWNSGALTSGVHTFPAVLQSSGLYSLSSV | TQAMDEADYYCQAWDSTTV |
| | | | | SYMYYADSVKG | NKRPSGIPER | VTVPSSSLGTQTYICNVNHKPSNTKVDKKVEPKSCDKT | VFGGGTKLTVLGQPKAAPS |
| | | | | RFTISRDNAKN | FSGSNSGKTA | HTCPPCPAPELLGGPSVFLFPPKPKDTLMISRTPEVTC | VTLFPPSSEELQANKATLV |
| | | | | SLYLQMNSLRA | TLTISGTQAM | VVVDVSHEDPEVKFNWYVDGVEVHNAKTKPREEQYNST | CLISDFYPGAVTVAWKADS |
| | | | | EDTAVYYCARD | DEADYYCQAW | YRVVSVLTVLHQDWLNGKEYKCKVSNKALPAPIEKTIS | SPVKAGVETTTPSKQSNNK |
| | | | | GDLVGPTYYFD | DSTTVVFGGG | KAKGQPREPQVYTLPPSREEMTKNQVSLTCLVKGFYPS | YAASSYLSLTPEQWKSHRS |
| | | | | YWGQGTLVTVS | TKLTVL | DIAVEWESNGQPENNYKTTPPVLDSDGSFFLYSKLTVD | YSCQVTHEGSTVEKTVAPT |
| | | | | S | | KSRWQQGNVFSCSVMHEALHNHYTQKSLSLSPGK | ECS |
| | | | | 235 | 236 | 237 | 238 |
| 8 | VG9B140 | IgG1 | Kappa | EVQLVESGGGL | EIVMTQSPGT | EVQLVESGGGLVKPGGSLRLSCAGSGFTFRSYDMNWVR | EIVMTQSPGTLSLSPGERA |
| | | | | VKPGGSLRLSC | LSLSPGERAT | QAPGKGLEWVSSISTSSGYIYYADSVKGRFTISRDNAK | TLSCRASQSVSSSYLAWYQ |
| | | | | AGSGFTFRSYD | LSCRASQSVS | NSLSLQMNSLRAEDTAVFYCARDRGIAVAGDYYYGMDV | QKPGQAPRLLIYGASSRAT |
| | | | | MNWVRQAPGKG | SSYLAWYQQK | WGQGTMVTVSSASTKGPSVFPLAPSSKSTSGGTAALGC | GIPDRFSGSGSGTDFTLTI |
| | | | | LEWVSSISTSS | PGQAPRLLIY | LVKDYFPEPVTVSWNSGALTSGVHTFPAVLQSSGLYSL | SRLEAEDFAVYYCQQYGSS |
| | | | | GYIYYADSVKG | GASSRATGIP | SSVVTVPSSSLGTQTYICNVNHKPSNTKVDKKVEPKSC | PPYTFGQGTKVEIKRTVAA |
| | | | | RFTISRDNAKN | DRFSGSGSGT | DKTHTCPPCPAPELLGGPSVFLFPPKPKDTLMISRTPE | PSVFIFPPSDEQLKSGTAS |
| | | | | SLSLQMNSLRA | DFTLTISRLE | VTCVVVDVSHEDPEVKFNWYVDGVEVHNAKTKPREEQY | VVCLLNNFYPREAKVQWKV |
| | | | | EDTAVFYCARD | AEDFAVYYCQ | NSTYRVVSVLTVLHQDWLNGKEYKCKVSNKALPAPIEK | DNALQSGNSQESVTEQDSK |
| | | | | RGIAVAGDYYY | QYGSSPPYTF | TISKAKGQPREPQVYTLPPSREEMTKNQVSLTCLVKGF | DSTYSLSSTLTLSKADYEK |
| | | | | GMDVWGQGTMV | GQGTKVEIK | YPSDIAVEWESNGQPENNYKTTPPVLDSDGSFFLYSKL | HKVYACEVTHQGLSSPVTK |
| | | | | TVSS | | TVDKSRWQQGNVFSCSVMHEALHNHYTQKSLSLSPGK | SFNRGEC |
| | | | | 269 | 270 | 271 | 272 |
| 9 | VG9B426 | IgG1 | Kappa | EVQLVQSGGGL | ETTLTQSPGT | EVQLVQSGGGLVKPGGSLRLSCSASGFTFSNYDMNWVR | ETTLTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSNYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTMVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKVEIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTMVT | GQGTKVEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 303 | 304 | 305 | 306 |
| 10 | VG9B46 | IgG1 | Kappa | EVQLVESGGGL | EIVLTQSPGT | EVQLVESGGGLVKPGGSLRLSCEASGFTFSINSMNWVR | EIVLTQSPGTLSLSPGERA |
| | | | | VKPGGSLRLSC | LSLSPGERAT | QAPGKGLEWVSSISSTSDYIFNADSVKGRFTISRDNAK | TLSCRTSQSVSRSYLGWYQ |
| | | | | EASGFTFSINS | LSCRTSQSVS | NSLYLQMNSLRAEDTAVYYCVRDDVFGAFDIWGQGTMV | QKPGQPPRLLIFGSSSRAT |
| | | | | MNWVRQAPGKG | RSYLGWYQQK | TVSSASTKGPSVFPLAPSSKSTSGGTAALGCLVKDYFP | GIPDRFSGSGSGTDFTLTI |
| | | | | LEWVSSISSTS | PGQPPRLLIF | EPVTVSWNSGALTSGVHTFPAVLQSSGLYSLSSVVTVP | SRLEPEDFAVYYCQQYSRS |
| | | | | DYIFNADSVKG | GSSSRATGIP | SSSLGTQTYICNVNHKPSNTKVDKKVEPKSCDKTHTCP | PLTFGGGTKVDIKRTVAAP |
| | | | | RFTISRDNAKN | DRFSGSGSGT | PCPAPELLGGPSVFLFPPKPKDTLMISRTPEVTCVVVD | SVFIFPPSDEQLKSGTASV |
| | | | | SLYLQMNSLRA | DFTLTISRLE | VSHEDPEVKFNWYVDGVEVHNAKTKPREEQYNSTYRVV | VCLLNNFYPREAKVQWKVD |
| | | | | EDTAVYYCVRD | PEDFAVYYCQ | SVLTVLHQDWLNGKEYKCKVSNKALPAPIEKTISKAKG | NALQSGNSQESVTEQDSKD |
| | | | | DVFGAFDIWGQ | QYSRSPLTFG | QPREPQVYTLPPSREEMTKNQVSLTCLVKGFYPSDIAV | STYSLSSTLTLSKADYEKH |
| | | | | GTMVTVSS | GGTKVDIK | EWESNGQPENNYKTTPPVLDSDGSFFLYSKLTVDKSRW | KVYACEVTHQGLSSPVTKS |
| | | | | | | QQGNVFSCSVMHEALHNHYTQKSLSLSPGK | FNRGEC |
| | | | | 337 | 338 | 339 | 340 |
| 11 | VG9B416 | IgG1 | Kappa | EVQLVESGGGL | EIVLTQSPGT | EVQLVESGGGLVKPGGSLRLSCSASGFTFSNYDMNWVR | EIVLTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSNYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTTVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKLEIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTTVT | GQGTKLEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 371 | 372 | 373 | 374 |
| 12 | VG9B69 | IgG1 | Kappa | EVQLVESGGGL | EIVMTQSPGT | EVQLVESGGGLVKPGGSLRLSCAASGFTFSSYDINWVR | EIVMTQSPGTLSLSPGERA |
| | | | | VKPGGSLRLSC | LSLSPGERAT | QAPGKGLEWVSSITSSSYYIYYADSVKGRFTISRDNAK | TLSCRASQSVSSSYLAWYQ |
| | | | | AASGFTFSSYD | LSCRASQSVS | NSLYLQMNSLRAEDTAVYYCARDLGVRGVDYYYYGLDV | QKPGQAPRLLIYGASSRAT |
| | | | | INWVRQAPGKG | SSYLAWYQQK | WGQGTMVTVSSASTKGPSVFPLAPSSKSTSGGTAALGC | GIPDRFSGSGSGTDFTLTI |
| | | | | LEWVSSITSSS | PGQAPRLLIY | LVKDYFPEPVTVSWNSGALTSGVHTFPAVLQSSGLYSL | SRLEPEDFAVYYCQQYGSS |
| | | | | YYIYYADSVKG | GASSRATGIP | SSVVTVPSSSLGTQTYICNVNHKPSNTKVDKKVEPKSC | PPYTFGQGTKVEIKRTVAA |
| | | | | RFTISRDNAKN | DRFSGSGSGT | DKTHTCPPCPAPELLGGPSVFLFPPKPKDTLMISRTPE | PSVFIFPPSDEQLKSGTAS |
| | | | | SLYLQMNSLRA | DFTLTISRLE | VTCVVVDVSHEDPEVKFNWYVDGVEVHNAKTKPREEQY | VVCLLNNFYPREAKVQWKV |
| | | | | EDTAVYYCARD | PEDFAVYYCQ | NSTYRVVSVLTVLHQDWLNGKEYKCKVSNKALPAPIEK | DNALQSGNSQESVTEQDSK |
| | | | | LGVRGVDYYYY | QYGSSPPYTF | TISKAKGQPREPQVYTLPPSREEMTKNQVSLTCLVKGF | DSTYSLSSTLTLSKADYEK |
| | | | | GLDVWGQGTMV | GQGTKVEIK | YPSDIAVEWESNGQPENNYKTTPPVLDSDGSFFLYSKL | HKVYACEVTHQGLSSPVTK |
| | | | | TVSS | | TVDKSRWQQGNVFSCSVMHEALHNHYTQKSLSLSPGK | SFNRGEC |
| | | | | 405 | 406 | 407 | 408 |
| 13 | VG9B415 | IgG1 | Kappa | EVQLVESGGGL | ETTLTQSPGT | EVQLVESGGGLVKPGGSLRLSCSASGFTFSNYDMNWVR | ETTLTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSNYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTTVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKLEIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTTVT | GQGTKLEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 439 | 440 | 441 | 442 |
| 14 | VG9B104 | IgG1 | Kappa | EVQLVESGGGL | EIVLTQSPGT | EVQLVESGGGLVKPGGSLRLSCAASGFTFSVYSMNWVR | EIVLTQSPGTLSLSPGERA |
| | | | | VKPGGSLRLSC | LSLSPGERAT | QAPGKGLEWVSSIGSSSSYIFYADSVKGRFTISRDNAK | TLSCRASQSVSSSYLAWYQ |
| | | | | AASGFTFSVYS | LSCRASQSVS | NSLYLQMNSLRAEDTAVYYCGRDHDYGGLDYWGQGTLV | QKPGQAPRLLIYGPSNRAT |
| | | | | MNWVRQAPGKG | SSYLAWYQQK | TVSSASTKGPSVFPLAPSSKSTSGGTAALGCLVKDYFP | DIPDRFSGTGSGTDFTLTI |
| | | | | LEWVSSIGSSS | PGQAPRLLIY | EPVTVSWNSGALTSGVHTFPAVLQSSGLYSLSSVVTVP | SRLEPEDFAVYYCQQYGRS |
| | | | | SYIFYADSVKG | GPSNRATDIP | SSSLGTQTYICNVNHKPSNTKVDKKVEPKSCDKTHTCP | PLTFGGGTKVEIKRTVAAP |
| | | | | RFTISRDNAKN | DRFSGTGSGT | PCPAPELLGGPSVFLFPPKPKDTLMISRTPEVTCVVVD | SVFIFPPSDEQLKSGTASV |
| | | | | SLYLQMNSLRA | DFTLTISRLE | VSHEDPEVKFNWYVDGVEVHNAKTKPREEQYNSTYRVV | VCLLNNFYPREAKVQWKVD |
| | | | | EDTAVYYCGRD | PEDFAVYYCQ | SVLTVLHQDWLNGKEYKCKVSNKALPAPIEKTISKAKG | NALQSGNSQESVTEQDSKD |
| | | | | HDYGGLDYWGQ | QYGRSPLTFG | QPREPQVYTLPPSREEMTKNQVSLTCLVKGFYPSDIAV | STYSLSSTLTLSKADYEKH |
| | | | | GTLVTVSS | GGTKVEIK | EWESNGQPENNYKTTPPVLDSDGSFFLYSKLTVDKSRW | KVYACEVTHQGLSSPVTKS |
| | | | | | | QQGNVFSCSVMHEALHNHYTQKSLSLSPGK | FNRGEC |
| | | | | 473 | 474 | 475 | 476 |
| 15 | VG9B198 | IgG1 | Lambda | QVQLQESGPGL | QSVLTQPPSV | QVQLQESGPGLVKPSETLSLTCTVSGDSISSIYWSWIR | QSVLTQPPSVSGAPGQRVT |
| | | | | VKPSETLSLTC | SGAPGQRVTI | QSAGKGLEWIGRIYTTDITNYNPSLKSRVTMSADTSKN | ISCTGSSSNIGAGYDVHWY |
| | | | | TVSGDSISSIY | SCTGSSSNIG | QLSLKLTSVTAADTAVYYCAKNGYSYGGFNYWGQGTLV | QRLPGTAPKLLIYGDSYRP |
| | | | | WSWIRQSAGKG | AGYDVHWYQR | TVSSASTKGPSVFPLAPSSKSTSGGTAALGCLVKDYFP | SGVPDRFSGSKSGTSASLA |
| | | | | LEWIGRIYTTD | LPGTAPKLLI | EPVTVSWNSGALTSGVHTFPAVLQSSGLYSLSSVVTVP | ITGLQAEDEADYYCQSYDS |
| | | | | ITNYNPSLKSR | YGDSYRPSGV | SSSLGTQTYICNVNHKPSNTKVDKKVEPKSCDKTHTCP | SLSVVVFGGGTKLTVLGQP |
| | | | | VTMSADTSKNQ | PDRFSGSKSG | PCPAPELLGGPSVFLFPPKPKDTLMISRTPEVTCVVVD | KAAPSVTLFPPSSEELQAN |
| | | | | LSLKLTSVTAA | TSASLAITGL | VSHEDPEVKFNWYVDGVEVHNAKTKPREEQYNSTYRVV | KATLVCLISDFYPGAVTVA |
| | | | | DTAVYYCAKNG | QAEDEADYYC | SVLTVLHQDWLNGKEYKCKVSNKALPAPIEKTISKAKG | WKADSSPVKAGVETTTPSK |
| | | | | YSYGGFNYWGQ | QSYDSSLSVV | QPREPQVYTLPPSREEMTKNQVSLTCLVKGFYPSDIAV | QSNNKYAASSYLSLTPEQW |
| | | | | GTLVTVSS | VFGGGTKLTV | EWESNGQPENNYKTTPPVLDSDGSFFLYSKLTVDKSRW | KSHRSYSCQVTHEGSTVEK |
| | | | | | L | QQGNVFSCSVMHEALHNHYTQKSLSLSPGK | TVAPTECS |
| | | | | 507 | 508 | 509 | 510 |
| 16 | VG9B463 | IgG1 | Lambda | EVQLVESGGGL | QPVLTQSSSA | EVQLVESGGGLVKPGGSLRLSCSASGFTFSNYDMNWVR | QPVLTQSSSASGTPGQRVT |
| | | | | VKPGGSLRLSC | SGTPGQRVTI | QAPGKGLEWVSSISSSSSYIYYADSVKGRFTISRDNAK | ISCSGSSSNIGSNTVNWYQ |
| | | | | SASGFTFSNYD | SCSGSSSNIG | NSLYLQMNSLRAEDTAVYHCARDVGVTTDYYYYGMDVW | QFPGTAPKLLIYTNTQRPS |
| | | | | MNWVRQAPGKG | SNTVNWYQQF | GQGTTVTVSSASTKGPSVFPLAPSSKSTSGGTAALGCL | GVPDRFSGSKSGTLVSLAI |
| | | | | LEWVSSISSSS | PGTAPKLLIY | VKDYFPEPVTVSWNSGALTSGVHTFPAVLQSSGLYSLS | SGLQSEDEADYYCAAWDDS |
| | | | | SYIYYADSVKG | TNTQRPSGVP | SVVTVPSSSLGTQTYICNVNHKPSNTKVDKKVEPKSCD | LNAWVFGGGTKLTVLGQPK |
| | | | | RFTISRDNAKN | DRFSGSKSGT | KTHTCPPCPAPELLGGPSVFLFPPKPKDTLMISRTPEV | AAPSVTLFPPSSEELQANK |
| | | | | SLYLQMNSLRA | LVSLAISGLQ | TCVVVDVSHEDPEVKFNWYVDGVEVHNAKTKPREEQYN | ATLVCLISDFYPGAVTVAW |
| | | | | EDTAVYHCARD | SEDEADYYCA | STYRVVSVLTVLHQDWLNGKEYKCKVSNKALPAPIEKT | KADSSPVKAGVETTTPSKQ |
| | | | | VGVTTDYYYYG | AWDDSLNAWV | ISKAKGQPREPQVYTLPPSREEMTKNQVSLTCLVKGFY | SNNKYAASSYLSLTPEQWK |
| | | | | MDVWGQGTTVT | FGGGTKLTVL | PSDIAVEWESNGQPENNYKTTPPVLDSDGSFFLYSKLT | SHRSYSCQVTHEGSTVEKT |
| | | | | VSS | | VDKSRWQQGNVFSCSVMHEALHNHYTQKSLSLSPGK | VAPTECS |
| | | | | 541 | 542 | 543 | 544 |
| 17 | VG9B469 | IgG1 | Kappa | EVQLVESGGGL | DIVMTQSPGT | EVQLVESGGGLVKPGGSLRLSCAASGFTFSHYDMNWVR | DIVMTQSPGTLSLSPGERA |
| | | | | VKPGGSLRLSC | LSLSPGERAT | QAPGKGLEWVSSISSSSSYIFYADSVKGRFTISRDNAK | TLSCRASQNVSSTYLAWYQ |
| | | | | AASGFTFSHYD | LSCRASQNVS | NSLLLQMNSLRAEDTAVYHCARDRGVGDTSDYYSFGLD | QKPGQAPRLLIYGACSRAT |
| | | | | MNWVRQAPGKG | STYLAWYQQK | VWGQGTMVTVSSASTKGPSVFPLAPSSKSTSGGTAALG | GIPDRFSGSGSGTDFTLTI |
| | | | | LEWVSSISSSS | PGQAPRLLIY | CLVKDYFPEPVTVSWNSGALTSGVHTFPAVLQSSGLYS | SRLEPEDYAVYYCQQYGSS |
| | | | | SYIFYADSVKG | GACSRATGIP | LSSVVTVPSSSLGTQTYICNVNHKPSNTKVDKKVEPKS | PPYTFGRGTKLEIKRTVAA |
| | | | | RFTISRDNAKN | DRFSGSGSGT | CDKTHTCPPCPAPELLGGPSVFLFPPKPKDTLMISRTP | PSVFIFPPSDEQLKSGTAS |
| | | | | SLLLQMNSLRA | DFTLTISRLE | EVTCVVVDVSHEDPEVKFNWYVDGVEVHNAKTKPREEQ | VVCLLNNFYPREAKVQWKV |
| | | | | EDTAVYHCARD | PEDYAVYYCQ | YNSTYRVVSVLTVLHQDWLNGKEYKCKVSNKALPAPIE | DNALQSGNSQESVTEQDSK |
| | | | | RGVGDTSDYYS | QYGSSPPYTF | KTISKAKGQPREPQVYTLPPSREEMTKNQVSLTCLVKG | DSTYSLSSTLTLSKADYEK |
| | | | | FGLDVWGQGTM | GRGTKLEIK | FYPSDIAVEWESNGQPENNYKTTPPVLDSDGSFFLYSK | HKVYACEVTHQGLSSPVTK |
| | | | | VTVSS | | LTVDKSRWQQGNVFSCSVMHEALHNHYTQKSLSLSPGK | SFNRGEC |
| | | | | 575 | 576 | 577 | 578 |
| 18 | VG9B428 | IgG1 | Kappa | EVQLVESGGGL | EIVMTQSPSS | EVQLVESGGGLVKPGGSLRLSCAASGFTFSNYGMNWVR | EIVMTQSPSSVSASVGDRV |
| | | | | VKPGGSLRLSC | VSASVGDRVT | QAPGKGLEWVSYISSGSSYKYYADSMKGRFTISRDNAM | TITCRASQGINSWLAWYQQ |
| | | | | AASGFTFSNYG | ITCRASQGIN | NLLYLQMNSLRPEDSAMYYCARDPVVTEYYYYGMDVWG | KPGNAPKLLIYAASSLQSG |
| | | | | MNWVRQAPGKG | SWLAWYQQKP | QGTMVTVSSASTKGPSVFPLAPSSKSTSGGTAALGCLV | VPSRFSGIGSGTDFIFTIS |
| | | | | LEWVSYISSGS | GNAPKLLIYA | KDYFPEPVTVSWNSGALTSGVHTFPAVLQSSGLYSLSS | SLQPEDFASYYCQQANSFP |
| | | | | SYKYYADSMKG | ASSLQSGVPS | VVTVPSSSLGTQTYICNVNHKPSNTKVDKKVEPKSCDK | WTFGQGTKVEIKRTVAAPS |
| | | | | RFTISRDNAMN | RFSGIGSGTD | THTCPPCPAPELLGGPSVFLFPPKPKDTLMISRTPEVT | VFIFPPSDEQLKSGTASVV |
| | | | | LLYLQMNSLRP | FIFTISSLQP | CVVVDVSHEDPEVKFNWYVDGVEVHNAKTKPREEQYNS | CLLNNFYPREAKVQWKVDN |
| | | | | EDSAMYYCARD | EDFASYYCQQ | TYRVVSVLTVLHQDWLNGKEYKCKVSNKALPAPIEKTI | ALQSGNSQESVTEQDSKDS |
| | | | | PVVTEYYYYGM | ANSFPWTFGQ | SKAKGQPREPQVYTLPPSREEMTKNQVSLTCLVKGFYP | TYSLSSTLTLSKADYEKHK |
| | | | | DVWGQGTMVTV | GTKVEIK | SDIAVEWESNGQPENNYKTTPPVLDSDGSFFLYSKLTV | VYACEVTHQGLSSPVTKSF |
| | | | | SS | | DKSRWQQGNVFSCSVMHEALHNHYTQKSLSLSPGK | NRGEC |
| | | | | 609 | 610 | 611 | 612 |
| 19 | VG9B430 | IgG1 | Kappa | EVQLLESGGGL | EIVLTQSPGT | EVQLLESGGGLVKPGGSLRLSCSASGFTFSSYDMNWVR | EIVLTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSSYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTMVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKLEIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTMVT | GQGTKLEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 643 | 644 | 645 | 646 |
| 20 | VG9B423 | IgG1 | Kappa | EVQLVESGGGL | ETTLTQSPGT | EVQLVESGGGLVKPGGSLRLSCSASGFTFSNYDMNWVR | ETTLTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSNYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTMVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKVEIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTMVT | GQGTKVEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 677 | 678 | 679 | 680 |
| 21 | VG9B98 | IgG1 | Kappa | EVQLVQSGGDL | EIVLTQSPGT | EVQLVQSGGDLVKSGGSLRLSCAASGFTFSSYSMYWVR | EIVLTQSPGTLSLSPGERA |
| | | | | VKSGGSLRLSC | LSLSPGERAT | QAPGKGLEWVSSIGSSSTYIFYADSVKGRFTISRDNAM | TLSCRASQSVSTSYLAWFQ |
| | | | | AASGFTFSSYS | LSCRASQSVS | TSLYLQMNRLGAEDSAVYYCARDGELGPFEYWGQGTLV | QKPGQAPRLLIFGTSSRAT |
| | | | | MYWVRQAPGKG | TSYLAWFQQK | TVSSASTKGPSVFPLAPSSKSTSGGTAALGCLVKDYFP | GIPDRFSGSGSGTDFTLTI |
| | | | | LEWVSSIGSSS | PGQAPRLLIF | EPVTVSWNSGALTSGVHTFPAVLQSSGLYSLSSVVTVP | SRLEPEDFAVYHCLQYGRS |
| | | | | TYIFYADSVKG | GTSSRATGIP | SSSLGTQTYICNVNHKPSNTKVDKKVEPKSCDKTHTCP | PLTFGGGTKVDIKRTVAAP |
| | | | | RFTISRDNAMT | DRFSGSGSGT | PCPAPELLGGPSVFLFPPKPKDTLMISRTPEVTCVVVD | SVFIFPPSDEQLKSGTASV |
| | | | | SLYLQMNRLGA | DFTLTISRLE | VSHEDPEVKFNWYVDGVEVHNAKTKPREEQYNSTYRVV | VCLLNNFYPREAKVQWKVD |
| | | | | EDSAVYYCARD | PEDFAVYHCL | SVLTVLHQDWLNGKEYKCKVSNKALPAPIEKTISKAKG | NALQSGNSQESVTEQDSKD |
| | | | | GELGPFEYWGQ | QYGRSPLTFG | QPREPQVYTLPPSREEMTKNQVSLTCLVKGFYPSDIAV | STYSLSSTLTLSKADYEKH |
| | | | | GTLVTVSS | GGTKVDIK | EWESNGQPENNYKTTPPVLDSDGSFFLYSKLTVDKSRW | KVYACEVTHQGLSSPVTKS |
| | | | | | | QQGNVFSCSVMHEALHNHYTQKSLSLSPGK | FNRGEC |
| | | | | 711 | 712 | 713 | 714 |
| 22 | VG9B73 | IgG1 | Kappa | EVQLVQSGGGL | EIVMTQSPGT | EVQLVQSGGGLVKPGGSLRLSCAASGFTFSNYDMNWVR | EIVMTQSPGTLSLSPGERA |
| | | | | VKPGGSLRLSC | LSLSPGERAT | QAPGKGLEWVSSISSSSSYIYYADSVKGRFTISRDNAR | TLSCRASQSFSSNYLAWYQ |
| | | | | AASGFTFSNYD | LSCRASQSFS | NSLFLQMSSLRAEDTAVYYCAREIGVTGTTYYQDYGMD | QKPGQAPRLLIYGASSRAT |
| | | | | MNWVRQAPGKG | SNYLAWYQQK | VWGQGTTVTVSSASTKGPSVFPLAPSSKSTSGGTAALG | GIPDRFSGSGSGTDFTLTI |
| | | | | LEWVSSISSSS | PGQAPRLLIY | CLVKDYFPEPVTVSWNSGALTSGVHTFPAVLQSSGLYS | SRLEPEDFALYYCQQYGSS |
| | | | | SYIYYADSVKG | GASSRATGIP | LSSVVTVPSSSLGTQTYICNVNHKPSNTKVDKKVEPKS | PPFTFGQGTKVEIKRTVAA |
| | | | | RFTISRDNARN | DRFSGSGSGT | CDKTHTCPPCPAPELLGGPSVFLFPPKPKDTLMISRTP | PSVFIFPPSDEQLKSGTAS |
| | | | | SLFLQMSSLRA | DFTLTISRLE | EVTCVVVDVSHEDPEVKFNWYVDGVEVHNAKTKPREEQ | VVCLLNNFYPREAKVQWKV |
| | | | | EDTAVYYCARE | PEDFALYYCQ | YNSTYRVVSVLTVLHQDWLNGKEYKCKVSNKALPAPIE | DNALQSGNSQESVTEQDSK |
| | | | | IGVTGTTYYQD | QYGSSPPFTF | KTISKAKGQPREPQVYTLPPSREEMTKNQVSLTCLVKG | DSTYSLSSTLTLSKADYEK |
| | | | | YGMDVWGQGTT | GQGTKVEIK | FYPSDIAVEWESNGQPENNYKTTPPVLDSDGSFFLYSK | HKVYACEVTHQGLSSPVTK |
| | | | | VTVSS | | LTVDKSRWQQGNVFSCSVMHEALHNHYTQKSLSLSPGK | SFNRGEC |
| | | | | 745 | 746 | 747 | 748 |
| 23 | VG9B133 | IgG1 | Kappa | EVQLVESGGGL | EIVMTQSPGT | EVQLVESGGGLVKPGGSLRLSCAAAGFTFSNYDMNWVR | EIVMTQSPGTLSLSPGERA |
| | | | | VKPGGSLRLSC | LSLSPGERAT | QVPGKGLEWVSSISSSSSYIYYADSVKGRFTISRDNAK | TLSCRASQSVSSSYLAWYQ |
| | | | | AAAGFTFSNYD | LSCRASQSVS | NSLYLQMNTLRAEDTAVYYCARDLGITGTTMDYYYGMD | QKPGQAPRLLIYGASSRAT |
| | | | | MNWVRQVPGKG | SSYLAWYQQK | VWGQGTMVTVSSASTKGPSVFPLAPSSKSTSGGTAALG | GIPDRFSGSGSGTDFTLTI |
| | | | | LEWVSSISSSS | PGQAPRLLIY | CLVKDYFPEPVTVSWNSGALTSGVHTFPAVLQSSGLYS | SRLEPEDFAVYYCQQYGSS |
| | | | | SYIYYADSVKG | GASSRATGIP | LSSVVTVPSSSLGTQTYICNVNHKPSNTKVDKKVEPKS | PPYTFGQGTKVDIKRTVAA |
| | | | | RFTISRDNAKN | DRFSGSGSGT | CDKTHTCPPCPAPELLGGPSVFLFPPKPKDTLMISRTP | PSVFIFPPSDEQLKSGTAS |
| | | | | SLYLQMNTLRA | DFTLTISRLE | EVTCVVVDVSHEDPEVKFNWYVDGVEVHNAKTKPREEQ | VVCLLNNFYPREAKVQWKV |
| | | | | EDTAVYYCARD | PEDFAVYYCQ | YNSTYRVVSVLTVLHQDWLNGKEYKCKVSNKALPAPIE | DNALQSGNSQESVTEQDSK |
| | | | | LGITGTTMDYY | QYGSSPPYTF | KTISKAKGQPREPQVYTLPPSREEMTKNQVSLTCLVKG | DSTYSLSSTLTLSKADYEK |
| | | | | YGMDVWGQGTM | GQGTKVDIK | FYPSDIAVEWESNGQPENNYKTTPPVLDSDGSFFLYSK | HKVYACEVTHQGLSSPVTK |
| | | | | VTVSS | | LTVDKSRWQQGNVFSCSVMHEALHNHYTQKSLSLSPGK | SFNRGEC |
| | | | | 779 | 780 | 781 | 782 |
| 24 | VG9B368 | IgG1 | Kappa | EVQLVESGGGL | EIVLTQSPGT | EVQLVESGGGLVKPGGSLRLSCAASGFTFNSYDMNWVR | EIVLTQSPGTLSLSPGERA |
| | | | | VKPGGSLRLSC | LSLSPGERAT | QAPGKGLEWVSSISSSSSYIYYADSVKGRCTISRDNAR | TLSCRASQSVSSSYLAWYQ |
| | | | | AASGFTFNSYD | LSCRASQSVS | NSLYLQMNSLRAEDTAIYYCARDRGIGGDYYSYAMDVW | QKPGQAPRLLIYGASSRAT |
| | | | | MNWVRQAPGKG | SSYLAWYQQK | GQGTMVTVSSASTKGPSVFPLAPSSKSTSGGTAALGCL | GIPDRFSGSGSGTDFTLTI |
| | | | | LEWVSSISSSS | PGQAPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKLEIKRTVAA |
| | | | | RCTISRDNARN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAIYYCARD | PEDFAVYYCQ | STYRVVSVLTVLHQDWLNGKEYKCKVSNKALPAPIEKT | DNALQSGNSQESVTEQDSK |
| | | | | RGIGGDYYSYA | QYGSSPPYTF | ISKAKGQPREPQVYTLPPSREEMTKNQVSLTCLVKGFY | DSTYSLSSTLTLSKADYEK |
| | | | | MDVWGQGTMVT | GQGTKLEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 813 | 814 | 815 | 816 |
| 25 | VG9B424 | IgG1 | Kappa | EVQLVESGGGL | ETTLTQSPGT | EVQLVESGGGLVKPGGSLRLSCSASGFTFSNYDMNWVR | ETTLTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSNYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTMVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKLEIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTMVT | GQGTKLEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 847 | 848 | 849 | 850 |
| 26 | VG9B427 | IgG1 | Kappa | QVQLVESGGGL | EIVMTQSPGT | QVQLVESGGGLVKPGGSLRLSCSASGFTFSNYDMNWVR | EIVMTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSNYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTTVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTRLEIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTTVT | GQGTRLEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 881 | 882 | 883 | 884 |
| 27 | VG9B417 | IgG1 | Kappa | EVQLVESGGGL | DIVMTQSPSS | EVQLVESGGGLVKPGGSLRLSCAASGFTFSNYGMNWVR | DIVMTQSPSSVSASVGDRV |
| | | | | VKPGGSLRLSC | VSASVGDRVT | QAPGKGLEWVSYISSGSSYKYYADSMKGRFTISRDNAM | TITCRASQGINSWLAWYQQ |
| | | | | AASGFTFSNYG | ITCRASQGIN | NLLYLQMNSLRPEDSAMYYCARDPVVTEYYYYGMDVWG | KPGNAPKLLIYAASSLQSG |
| | | | | MNWVRQAPGKG | SWLAWYQQKP | QGTTVTVSSASTKGPSVFPLAPSSKSTSGGTAALGCLV | VPSRFSGIGSGTDFIFTIS |
| | | | | LEWVSYISSGS | GNAPKLLIYA | KDYFPEPVTVSWNSGALTSGVHTFPAVLQSSGLYSLSS | SLQPEDFASYYCQQANSFP |
| | | | | SYKYYADSMKG | ASSLQSGVPS | VVTVPSSSLGTQTYICNVNHKPSNTKVDKKVEPKSCDK | WTFGQGTKVEIKRTVAAPS |
| | | | | RFTISRDNAMN | RFSGIGSGTD | THTCPPCPAPELLGGPSVFLFPPKPKDTLMISRTPEVT | VFIFPPSDEQLKSGTASVV |
| | | | | LLYLQMNSLRP | FIFTISSLQP | CVVVDVSHEDPEVKFNWYVDGVEVHNAKTKPREEQYNS | CLLNNFYPREAKVQWKVDN |
| | | | | EDSAMYYCARD | EDFASYYCQQ | TYRVVSVLTVLHQDWLNGKEYKCKVSNKALPAPIEKTI | ALQSGNSQESVTEQDSKDS |
| | | | | PVVTEYYYYGM | ANSFPWTFGQ | SKAKGQPREPQVYTLPPSREEMTKNQVSLTCLVKGFYP | TYSLSSTLTLSKADYEKHK |
| | | | | DVWGQGTTVTV | GTKVEIK | SDIAVEWESNGQPENNYKTTPPVLDSDGSFFLYSKLTV | VYACEVTHQGLSSPVTKSF |
| | | | | SS | | DKSRWQQGNVFSCSVMHEALHNHYTQKSLSLSPGK | NRGEC |
| | | | | 915 | 916 | 917 | 918 |
| 28 | VG9B58 | IgG1 | Kappa | QVQLQESGPGL | DIQMTQSPSA | QVQLQESGPGLVKPSETLSLTCTVSGGSIKSSYWTWIR | DIQMTQSPSAMSASVGDRV |
| | | | | VKPSETLSLTC | MSASVGDRVT | QPPGKGLEWIGYMFYLGSTNYNPSLKSRVTMSIDTSRN | TITCRASQDITNYLAWFQQ |
| | | | | TVSGGSIKSSY | ITCRASQDIT | QFSLKLSSVTAADTAVYYCSRERPVLDAFDIWGQGTMV | KPGKVPKRLIYAASSLQGG |
| | | | | WTWIRQPPGKG | NYLAWFQQKP | TVSSASTKGPSVFPLAPSSKSTSGGTAALGCLVKDYFP | VPSRFSGSGSGTEFTLTIS |
| | | | | LEWIGYMFYLG | GKVPKRLIYA | EPVTVSWNSGALTSGVHTFPAVLQSSGLYSLSSVVTVP | SLQPEDFSTYYCLQHDTYP |
| | | | | STNYNPSLKSR | ASSLQGGVPS | SSSLGTQTYICNVNHKPSNTKVDKKVEPKSCDKTHTCP | YTFGQGTKVDIKRTVAAPS |
| | | | | VTMSIDTSRNQ | RFSGSGSGTE | PCPAPELLGGPSVFLFPPKPKDTLMISRTPEVTCVVVD | VFIFPPSDEQLKSGTASVV |
| | | | | FSLKLSSVTAA | FTLTISSLQP | VSHEDPEVKFNWYVDGVEVHNAKTKPREEQYNSTYRVV | CLLNNFYPREAKVQWKVDN |
| | | | | DTAVYYCSRER | EDFSTYYCLQ | SVLTVLHQDWLNGKEYKCKVSNKALPAPIEKTISKAKG | ALQSGNSQESVTEQDSKDS |
| | | | | PVLDAFDIWGQ | HDTYPYTFGQ | QPREPQVYTLPPSREEMTKNQVSLTCLVKGFYPSDIAV | TYSLSSTLTLSKADYEKHK |
| | | | | GTMVTVSS | GTKVDIK | EWESNGQPENNYKTTPPVLDSDGSFFLYSKLTVDKSRW | VYACEVTHQGLSSPVTKSF |
| | | | | | | QQGNVFSCSVMHEALHNHYTQKSLSLSPGK | NRGEC |
| | | | | 949 | 950 | 951 | 952 |
| 29 | VG9B419 | IgG1 | Kappa | EVQLVESGGGL | EIVLTQSPGT | EVQLVESGGGLVKPGGSLRLSCSASGFTFSNYDMNWVR | EIVLTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSNYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTTVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKVEIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTTVT | GQGTKVEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 983 | 984 | 985 | 986 |
| 30 | VG9B425 | IgG1 | Kappa | EVQLLESGGGL | ETTLTQSPGT | EVQLLESGGGLVKPGGSLRLSCSASGFTFSNYDMNWVR | ETTLTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSNYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTMVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKVEIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTMVT | GQGTKVEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 1017 | 1018 | 1019 | 1020 |
| 31 | VG9B143 | IgG1 | Lambda | EVQLVESGGGL | QSALTQPASV | EVQLVESGGGLVQPGGSLRLSCAASGFTFSTYGMNWVR | QSALTQPASVSGSPGQSIT |
| | | | | VQPGGSLRLSC | SGSPGQSITI | QAPGKGLEWISYISTSSYTIYYSDSVKGRFTVSRDNAK | ISCTGTSSDVGGYNYVSWY |
| | | | | AASGFTFSTYG | SCTGTSSDVG | NSLYLQMNSLRDEDTAVYYCAREGDWWYFDLWGRGTTV | QQHPGKAPKLMIYEVSNRP |
| | | | | MNWVRQAPGKG | GYNYVSWYQQ | TVSSASTKGPSVFPLAPSSKSTSGGTAALGCLVKDYFP | SGVSYRFSGSKSGNTASLT |
| | | | | LEWISYISTSS | HPGKAPKLMI | EPVTVSWNSGALTSGVHTFPAVLQSSGLYSLSSVVTVP | ISGLQAEDEADYYCSSYTS |
| | | | | YTIYYSDSVKG | YEVSNRPSGV | SSSLGTQTYICNVNHKPSNTKVDKKVEPKSCDKTHTCP | SSTLVFGGGTKLTVLGQPK |
| | | | | RFTVSRDNAKN | SYRFSGSKSG | PCPAPELLGGPSVFLFPPKPKDTLMISRTPEVTCVVVD | AAPSVTLFPPSSEELQANK |
| | | | | SLYLQMNSLRD | NTASLTISGL | VSHEDPEVKFNWYVDGVEVHNAKTKPREEQYNSTYRVV | ATLVCLISDFYPGAVTVAW |
| | | | | EDTAVYYCARE | QAEDEADYYC | SVLTVLHQDWLNGKEYKCKVSNKALPAPIEKTISKAKG | KADSSPVKAGVETTTPSKQ |
| | | | | GDWWYFDLWGR | SSYTSSSTLV | QPREPQVYTLPPSREEMTKNQVSLTCLVKGFYPSDIAV | SNNKYAASSYLSLTPEQWK |
| | | | | GTTVTVSS | FGGGTKLTVL | EWESNGQPENNYKTTPPVLDSDGSFFLYSKLTVDKSRW | SHRSYSCQVTHEGSTVEKT |
| | | | | | | QQGNVFSCSVMHEALHNHYTQKSLSLSPGK | VAPTECS |
| | | | | 1051 | 1052 | 1053 | 1054 |
| 32 | VG9B418 | IgG1 | Kappa | EVQLVQSGGGL | EIVLTQSPGT | EVQLVQSGGGLVKPGGSLRLSCSASGFTFSNYDMNWVR | EIVLTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSNYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTMVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKLEIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTMVT | GQGTKLEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 1085 | 1086 | 1087 | 1088 |
| 33 | VG9B472 | IgG1 | Kappa | QVQLVESGGGL | EIVLTQSPSS | QVQLVESGGGLVKPGGSLRLSCAASGFTFSHYDMNWVR | EIVLTQSPSSLSASVGDRV |
| | | | | VKPGGSLRLSC | LSASVGDRVT | QAPGKGLEWVSSISSSSSYIFYADSVKGRFTISRDNAK | TITCRASQGIATYLAWYQQ |
| | | | | AASGFTFSHYD | ITCRASQGIA | NSLLLQMNSLRAEDTAVYHCARDRGVGDTSDYYSFGLD | KPGKVPNLLIYAASTLQSG |
| | | | | MNWVRQAPGKG | TYLAWYQQKP | VWGQGTTVTVSSASTKGPSVFPLAPSSKSTSGGTAALG | VPSRFSGSGSGTDFTLTIS |
| | | | | LEWVSSISSSS | GKVPNLLIYA | CLVKDYFPEPVTVSWNSGALTSGVHTFPAVLQSSGLYS | SLQPEDVATYYCQKYNSAP |
| | | | | SYIFYADSVKG | ASTLQSGVPS | LSSVVTVPSSSLGTQTYICNVNHKPSNTKVDKKVEPKS | PWTFGQGTKVEIKRTVAAP |
| | | | | RFTISRDNAKN | RFSGSGSGTD | CDKTHTCPPCPAPELLGGPSVFLFPPKPKDTLMISRTP | SVFIFPPSDEQLKSGTASV |
| | | | | SLLLQMNSLRA | FTLTISSLQP | EVTCVVVDVSHEDPEVKFNWYVDGVEVHNAKTKPREEQ | VCLLNNFYPREAKVQWKVD |
| | | | | EDTAVYHCARD | EDVATYYCQK | YNSTYRVVSVLTVLHQDWLNGKEYKCKVSNKALPAPIE | NALQSGNSQESVTEQDSKD |
| | | | | RGVGDTSDYYS | YNSAPPWTFG | KTISKAKGQPREPQVYTLPPSREEMTKNQVSLTCLVKG | STYSLSSTLTLSKADYEKH |
| | | | | FGLDVWGQGTT | QGTKVEIK | FYPSDIAVEWESNGQPENNYKTTPPVLDSDGSFFLYSK | KVYACEVTHQGLSSPVTKS |
| | | | | VTVSS | | LTVDKSRWQQGNVFSCSVMHEALHNHYTQKSLSLSPGK | FNRGEC |
| | | | | 1119 | 1120 | 1121 | 1122 |
| 34 | VG9B421 | IgG1 | Kappa | EVQLVESGGGL | DIQMTQSPSS | EVQLVESGGGLVKPGGSLRLSCAASGFTFSNYGMNWVR | DIQMTQSPSSVSASVGDRV |
| | | | | VKPGGSLRLSC | VSASVGDRVT | QAPGKGLEWVSYISSGSSYKYYADSMKGRFTISRDNAM | TITCRASQGINSWLAWYQQ |
| | | | | AASGFTFSNYG | ITCRASQGIN | NLLYLQMNSLRPEDSAMYYCARDPVVTEYYYYGMDVWG | KPGNAPKLLIYAASSLQSG |
| | | | | MNWVRQAPGKG | SWLAWYQQKP | QGTMVTVSSASTKGPSVFPLAPSSKSTSGGTAALGCLV | VPSRFSGIGSGTDFIFTIS |
| | | | | LEWVSYISSGS | GNAPKLLIYA | KDYFPEPVTVSWNSGALTSGVHTFPAVLQSSGLYSLSS | SLQPEDFASYYCQQANSFP |
| | | | | SYKYYADSMKG | ASSLQSGVPS | VVTVPSSSLGTQTYICNVNHKPSNTKVDKKVEPKSCDK | WTFGQGTRLEIKRTVAAPS |
| | | | | RFTISRDNAMN | RFSGIGSGTD | THTCPPCPAPELLGGPSVFLFPPKPKDTLMISRTPEVT | VFIFPPSDEQLKSGTASVV |
| | | | | LLYLQMNSLRP | FIFTISSLQP | CVVVDVSHEDPEVKFNWYVDGVEVHNAKTKPREEQYNS | CLLNNFYPREAKVQWKVDN |
| | | | | EDSAMYYCARD | EDFASYYCQQ | TYRVVSVLTVLHQDWLNGKEYKCKVSNKALPAPIEKTI | ALQSGNSQESVTEQDSKDS |
| | | | | PVVTEYYYYGM | ANSFPWTFGQ | SKAKGQPREPQVYTLPPSREEMTKNQVSLTCLVKGFYP | TYSLSSTLTLSKADYEKHK |
| | | | | DVWGQGTMVTV | GTRLEIK | SDIAVEWESNGQPENNYKTTPPVLDSDGSFFLYSKLTV | VYACEVTHQGLSSPVTKSF |
| | | | | SS | | DKSRWQQGNVFSCSVMHEALHNHYTQKSLSLSPGK | NRGEC |
| | | | | 1153 | 1154 | 1155 | 1156 |
| 35 | VG9B88 | IgG1 | Kappa | EVQLVESGGGL | EIVLTQSPLS | EVQLVESGGGLVKPGGSLRLSCAVSGFTFSSYTMNWVR | EIVLTQSPLSLPVTPGEPA |
| | | | | VKPGGSLRLSC | LPVTPGEPAS | QAPGKGLEWVSSISTSSSYIDYADSVKGRFTISRDNAK | SISCRSSQSLLNSDDGNTY |
| | | | | AVSGFTFSSYT | ISCRSSQSLL | KSLYLQMNSLRAEDTAVYYCARDGDMVAPIKGSFDYWG | LDWYLQKPGQSPQLLIYTL |
| | | | | MNWVRQAPGKG | NSDDGNTYLD | QGTLVTVSSASTKGPSVFPLAPSSKSTSGGTAALGCLV | SYRASGVPDRFSGSGSGTD |
| | | | | LEWVSSISTSS | WYLQKPGQSP | KDYFPEPVTVSWNSGALTSGVHTFPAVLQSSGLYSLSS | FTLKISRVEAEDVGVYYCM |
| | | | | SYIDYADSVKG | QLLIYTLSYR | VVTVPSSSLGTQTYICNVNHKPSNTKVDKKVEPKSCDK | QRIEFPITFGQGTRLEIKR |
| | | | | RFTISRDNAKK | ASGVPDRFSG | THTCPPCPAPELLGGPSVFLFPPKPKDTLMISRTPEVT | TVAAPSVFIFPPSDEQLKS |
| | | | | SLYLQMNSLRA | SGSGTDFTLK | CVVVDVSHEDPEVKFNWYVDGVEVHNAKTKPREEQYNS | GTASVVCLLNNFYPREAKV |
| | | | | EDTAVYYCARD | ISRVEAEDVG | TYRVVSVLTVLHQDWLNGKEYKCKVSNKALPAPIEKTI | QWKVDNALQSGNSQESVTE |
| | | | | GDMVAPIKGSF | VYYCMQRIEF | SKAKGQPREPQVYTLPPSREEMTKNQVSLTCLVKGFYP | QDSKDSTYSLSSTLTLSKA |
| | | | | DYWGQGTLVTV | PITFGQGTRL | SDIAVEWESNGQPENNYKTTPPVLDSDGSFFLYSKLTV | DYEKHKVYACEVTHQGLSS |
| | | | | SS | EIK | DKSRWQQGNVFSCSVMHEALHNHYTQKSLSLSPGK | PVTKSFNRGEC |
| | | | | 1187 | 1188 | 1189 | 1190 |
| 36 | VG9B384 | IgG1 | Lambda | QVQLQESGPAL | SYELMQPPSV | QVQLQESGPALVKPTQTLTLTCTFSGFSLSSSGMCVSW | SYELMQPPSVSVSPGQTAS |
| | | | | VKPTQTLTLTC | SVSPGQTASI | IRQPPGKALEWLTLIDWFDDKYYSTSLKTRLTISKDTS | ITCSGNELGDKYASWYQQQ |
| | | | | TFSGFSLSSSG | TCSGNELGDK | KNQVVLTMTNMDPVDTATYYCARIRGTGAYYYGLDVWG | PGQSPVLVIYQDNKRPSGI |
| | | | | MCVSWIRQPPG | YASWYQQQPG | QGTMVTVSSASTKGPSVFPLAPSSKSTSGGTAALGCLV | PERFSGSNSGNTATLTISG |
| | | | | KALEWLTLIDW | QSPVLVIYQD | KDYFPEPVTVSWNSGALTSGVHTFPAVLQSSGLYSLSS | TQAMDEADYYCQAWDSSKV |
| | | | | FDDKYYSTSLK | NKRPSGIPER | VVTVPSSSLGTQTYICNVNHKPSNTKVDKKVEPKSCDK | VFGGGTKLTVLGQPKAAPS |
| | | | | TRLTISKDTSK | FSGSNSGNTA | THTCPPCPAPELLGGPSVFLFPPKPKDTLMISRTPEVT | VTLFPPSSEELQANKATLV |
| | | | | NQVVLTMTNMD | TLTISGTQAM | CVVVDVSHEDPEVKFNWYVDGVEVHNAKTKPREEQYNS | CLISDFYPGAVTVAWKADS |
| | | | | PVDTATYYCAR | DEADYYCQAW | TYRVVSVLTVLHQDWLNGKEYKCKVSNKALPAPIEKTI | SPVKAGVETTTPSKQSNNK |
| | | | | IRGTGAYYYGL | DSSKVVFGGG | SKAKGQPREPQVYTLPPSREEMTKNQVSLTCLVKGFYP | YAASSYLSLTPEQWKSHRS |
| | | | | DVWGQGTMVTV | TKLTVL | SDIAVEWESNGQPENNYKTTPPVLDSDGSFFLYSKLTV | YSCQVTHEGSTVEKTVAPT |
| | | | | SS | | DKSRWQQGNVFSCSVMHEALHNHYTQKSLSLSPGK | ECS |
| | | | | 1221 | 1222 | 1223 | 1224 |
| 37 | VG9B413 | IgG1 | Kappa | EVQLVESGGGL | EIVMTQSPGT | EVQLVESGGGLVKPGGSLRLSCSASGFTFSNYDMNWVR | EIVMTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSNYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTMVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKVEIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTMVT | GQGTKVEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 1255 | 1256 | 1257 | 1258 |
| 38 | VG9B36 | IgG1 | Kappa | QVQLVESGGGL | EIVLTQSPSS | QVQLVESGGGLVKPGGSLRLSCASSGFTFSSYGMYWVR | EIVLTQSPSSLSASVGDRV |
| | | | | VKPGGSLRLSC | LSASVGDRVT | QAPGKGLEWVSSISTGSSYIYYADSVKGRFTISRDNAK | TITCQASQDISNYLNWYQQ |
| | | | | ASSGFTFSSYG | ITCQASQDIS | NSLYLQMNSLRAEDTAVYYCARDKGLAVTGYIMDVWGQ | KPGKAPKLLIYDASNLETG |
| | | | | MYWVRQAPGKG | NYLNWYQQKP | GTTVTVSSASTKGPSVFPLAPSSKSTSGGTAALGCLVK | VPSRFSGSGSGTDFTFTIS |
| | | | | LEWVSSISTGS | GKAPKLLIYD | DYFPEPVTVSWNSGALTSGVHTFPAVLQSSGLYSLSSV | SLQPEDIAIYYCQQYDNLP |
| | | | | SYIYYADSVKG | ASNLETGVPS | VTVPSSSLGTQTYICNVNHKPSNTKVDKKVEPKSCDKT | MYTFGQGTKVEIKRTVAAP |
| | | | | RFTISRDNAKN | RFSGSGSGTD | HTCPPCPAPELLGGPSVFLFPPKPKDTLMISRTPEVTC | SVFIFPPSDEQLKSGTASV |
| | | | | SLYLQMNSLRA | FTFTISSLQP | VVVDVSHEDPEVKFNWYVDGVEVHNAKTKPREEQYNST | VCLLNNFYPREAKVQWKVD |
| | | | | EDTAVYYCARD | EDIAIYYCQQ | YRVVSVLTVLHQDWLNGKEYKCKVSNKALPAPIEKTIS | NALQSGNSQESVTEQDSKD |
| | | | | KGLAVTGYIMD | YDNLPMYTFG | KAKGQPREPQVYTLPPSREEMTKNQVSLTCLVKGFYPS | STYSLSSTLTLSKADYEKH |
| | | | | VWGQGTTVTVS | QGTKVEIK | DIAVEWESNGQPENNYKTTPPVLDSDGSFFLYSKLTVD | KVYACEVTHQGLSSPVTKS |
| | | | | S | | KSRWQQGNVFSCSVMHEALHNHYTQKSLSLSPGK | FNRGEC |
| | | | | 1289 | 1290 | 1291 | 1292 |
| 39 | VG9B403 | IgG1 | Kappa | EVQLVESGGGL | DIVMTQSPGT | EVQLVESGGGLVKPGGSLRLSCSASGFTFSNYDMNWVR | DIVMTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSNYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTTVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKVEIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTTVT | GQGTKVEIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 1323 | 1324 | 1325 | 1326 |
| 40 | VG9B191 | IgG1 | Lambda | EVQLVQSGGGL | QSVLTQPPSV | EVQLVQSGGGLVKPGGSLRLSCAASGFTFSRYSMNWVR | QSVLTQPPSVSAAPGQKVT |
| | | | | VKPGGSLRLSC | SAAPGQKVTI | QAPGKGLEGVSSISSSSSYIYYADSVKGRFTISRDNAK | ISCSGSSSNIGNNYVSWYQ |
| | | | | AASGFTFSRYS | SCSGSSSNIG | NSLYLQMNSLRAEDTAVYYCARDGPTVNWDYYFDLWGR | QLPGTAPKLLIYDNNKRPS |
| | | | | MNWVRQAPGKG | NNYVSWYQQL | GTLVTVSSASTKGPSVFPLAPSSKSTSGGTAALGCLVK | GIPDRFSGSKSGTSATLGI |
| | | | | LEGVSSISSSS | PGTAPKLLIY | DYFPEPVTVSWNSGALTSGVHTFPAVLQSSGLYSLSSV | TGLQTGDEADYYCGTWDSS |
| | | | | SYIYYADSVKG | DNNKRPSGIP | VTVPSSSLGTQTYICNVNHKPSNTKVDKKVEPKSCDKT | LSTSVVFGGGTKLTVLGQP |
| | | | | RFTISRDNAKN | DRFSGSKSGT | HTCPPCPAPELLGGPSVFLFPPKPKDTLMISRTPEVTC | KAAPSVTLFPPSSEELQAN |
| | | | | SLYLQMNSLRA | SATLGITGLQ | VVVDVSHEDPEVKFNWYVDGVEVHNAKTKPREEQYNST | KATLVCLISDFYPGAVTVA |
| | | | | EDTAVYYCARD | TGDEADYYCG | YRVVSVLTVLHQDWLNGKEYKCKVSNKALPAPIEKTIS | WKADSSPVKAGVETTTPSK |
| | | | | GPTVNWDYYFD | TWDSSLSTSV | KAKGQPREPQVYTLPPSREEMTKNQVSLTCLVKGFYPS | QSNNKYAASSYLSLTPEQW |
| | | | | LWGRGTLVTVS | VFGGGTKLTV | DIAVEWESNGQPENNYKTTPPVLDSDGSFFLYSKLTVD | KSHRSYSCQVTHEGSTVEK |
| | | | | S | L | KSRWQQGNVFSCSVMHEALHNHYTQKSLSLSPGK | TVAPTECS |
| | | | | 1357 | 1358 | 1359 | 1360 |
| 41 | VG9B44 | IgG1 | Kappa | QVQLQQSGPGL | DIQMTQSPSF | QVQLQQSGPGLVKPSQTLSLTCAISGDSVSSNSAAWNW | DIQMTQSPSFLSASVGDRV |
| | | | | VKPSQTLSLTC | LSASVGDRVT | IRQSPSRGLEWLGRTYYRSKWYNDYPISVKSRISINPD | TITCRASQGISSYLAWYQQ |
| | | | | AISGDSVSSNS | ITCRASQGIS | TSKNQFSLQLNSVTPEDTAVYYCARESGSYYTDGFDIW | KPGKAPKLLIYAASTLQSG |
| | | | | AAWNWIRQSPS | SYLAWYQQKP | GQGTMVTVSSASTKGPSVFPLAPSSKSTSGGTAALGCL | VPSRFSGSGSGTEFTLTIS |
| | | | | RGLEWLGRTYY | GKAPKLLIYA | VKDYFPEPVTVSWNSGALTSGVHTFPAVLQSSGLYSLS | SLQPEDFATYYCQEFNSYP |
| | | | | RSKWYNDYPIS | ASTLQSGVPS | SVVTVPSSSLGTQTYICNVNHKPSNTKVDKKVEPKSCD | YTFGQGTKLEIKRTVAAPS |
| | | | | VKSRISINPDT | RFSGSGSGTE | KTHTCPPCPAPELLGGPSVFLFPPKPKDTLMISRTPEV | VFIFPPSDEQLKSGTASVV |
| | | | | SKNQFSLQLNS | FTLTISSLQP | TCVVVDVSHEDPEVKFNWYVDGVEVHNAKTKPREEQYN | CLLNNFYPREAKVQWKVDN |
| | | | | VTPEDTAVYYC | EDFATYYCQE | STYRVVSVLTVLHQDWLNGKEYKCKVSNKALPAPIEKT | ALQSGNSQESVTEQDSKDS |
| | | | | ARESGSYYTDG | FNSYPYTFGQ | ISKAKGQPREPQVYTLPPSREEMTKNQVSLTCLVKGFY | TYSLSSTLTLSKADYEKHK |
| | | | | FDIWGQGTMVT | GTKLEIK | PSDIAVEWESNGQPENNYKTTPPVLDSDGSFFLYSKLT | VYACEVTHQGLSSPVTKSF |
| | | | | VSS | | VDKSRWQQGNVFSCSVMHEALHNHYTQKSLSLSPGK | NRGEC |
| | | | | 1391 | 1392 | 1393 | 1394 |
| 42 | VG9B67 | IgG1 | Kappa | EVQLVESGGGL | DIVMTQTPLS | EVQLVESGGGLVKPGGSLRLSCAASGFTFSSYTMNWVR | DIVMTQTPLSLPVTPGEPA |
| | | | | VKPGGSLRLSC | LPVTPGEPAS | QAPGKGLEWVSSISSSSSYIDYAESVKGRFTISRDNAK | SISCRSSQSLLDSDAGNTY |
| | | | | AASGFTFSSYT | ISCRSSQSLL | NSLYLQMNSLRAEDTAVYYCARDGDILATIRGSFDYWG | LDWYLQKPGQSPQLLIYTL |
| | | | | MNWVRQAPGKG | DSDAGNTYLD | QGTTVTVSSASTKGPSVFPLAPSSKSTSGGTAALGCLV | SYRASGVPDRFSGSGSGTD |
| | | | | LEWVSSISSSS | WYLQKPGQSP | KDYFPEPVTVSWNSGALTSGVHTFPAVLQSSGLYSLSS | FTLKISRVEAEDVAIYYCM |
| | | | | SYIDYAESVKG | QLLIYTLSYR | VVTVPSSSLGTQTYICNVNHKPSNTKVDKKVEPKSCDK | QRIEFPITFGQGTKVEIKR |
| | | | | RFTISRDNAKN | ASGVPDRFSG | THTCPPCPAPELLGGPSVFLFPPKPKDTLMISRTPEVT | TVAAPSVFIFPPSDEQLKS |
| | | | | SLYLQMNSLRA | SGSGTDFTLK | CVVVDVSHEDPEVKFNWYVDGVEVHNAKTKPREEQYNS | GTASVVCLLNNFYPREAKV |
| | | | | EDTAVYYCARD | ISRVEAEDVA | TYRVVSVLTVLHQDWLNGKEYKCKVSNKALPAPIEKTI | QWKVDNALQSGNSQESVTE |
| | | | | GDILATIRGSF | IYYCMQRIEF | SKAKGQPREPQVYTLPPSREEMTKNQVSLTCLVKGFYP | QDSKDSTYSLSSTLTLSKA |
| | | | | DYWGQGTTVTV | PITFGQGTKV | SDIAVEWESNGQPENNYKTTPPVLDSDGSFFLYSKLTV | DYEKHKVYACEVTHQGLSS |
| | | | | SS | EIK | DKSRWQQGNVFSCSVMHEALHNHYTQKSLSLSPGK | PVTKSFNRGEC |
| | | | | 1425 | 1426 | 1427 | 1428 |
| 43 | VG9B402 | IgG1 | Kappa | EVQLVQSGGGL | DIQMTQSPSS | EVQLVQSGGGLVKPGGSLRLSCAASGFTFSNYGMNWVR | DIQMTQSPSSVSASVGDRV |
| | | | | VKPGGSLRLSC | VSASVGDRVT | QAPGKGLEWVSYISSGSSYKYYADSMKGRFTISRDNAM | TITCRASQGINSWLAWYQQ |
| | | | | AASGFTFSNYG | ITCRASQGIN | NLLYLQMNSLRPEDSAMYYCARDPVVTEYYYYGMDVWG | KPGNAPKLLIYAASSLQSG |
| | | | | MNWVRQAPGKG | SWLAWYQQKP | QGTMVTVSSASTKGPSVFPLAPSSKSTSGGTAALGCLV | VPSRFSGIGSGTDFIFTIS |
| | | | | LEWVSYISSGS | GNAPKLLIYA | KDYFPEPVTVSWNSGALTSGVHTFPAVLQSSGLYSLSS | SLQPEDFASYYCQQANSFP |
| | | | | SYKYYADSMKG | ASSLQSGVPS | VVTVPSSSLGTQTYICNVNHKPSNTKVDKKVEPKSCDK | WTFGQGTKVDIKRTVAAPS |
| | | | | RFTISRDNAMN | RFSGIGSGTD | THTCPPCPAPELLGGPSVFLFPPKPKDTLMISRTPEVT | VFIFPPSDEQLKSGTASVV |
| | | | | LLYLQMNSLRP | FIFTISSLQP | CVVVDVSHEDPEVKFNWYVDGVEVHNAKTKPREEQYNS | CLLNNFYPREAKVQWKVDN |
| | | | | EDSAMYYCARD | EDFASYYCQQ | TYRVVSVLTVLHQDWLNGKEYKCKVSNKALPAPIEKTI | ALQSGNSQESVTEQDSKDS |
| | | | | PVVTEYYYYGM | ANSFPWTFGQ | SKAKGQPREPQVYTLPPSREEMTKNQVSLTCLVKGFYP | TYSLSSTLTLSKADYEKHK |
| | | | | DVWGQGTMVTV | GTKVDIK | SDIAVEWESNGQPENNYKTTPPVLDSDGSFFLYSKLTV | VYACEVTHQGLSSPVTKSF |
| | | | | SS | | DKSRWQQGNVFSCSVMHEALHNHYTQKSLSLSPGK | NRGEC |
| | | | | 1459 | 1460 | 1461 | 1462 |
| 44 | VG9B127 | IgG1 | Kappa | QVQLVESGGGL | EIVMTQSPLS | QVQLVESGGGLVKPGGSLRLSCTASGFTFGAYTMNWVR | EIVMTQSPLSLPVTPGEPA |
| | | | | VKPGGSLRLSC | LPVTPGEPAS | QAPGKGLEWVSSISSSSSYIDFAESVKGRFTISRDNAK | SISCRSSQSLLDSDDGNTY |
| | | | | TASGFTFGAYT | ISCRSSQSLL | NSLYLQMISLRAEDTAVYYCARDGDIVSTIRGSFDYWG | LDWYLQKPGQSPQLLIYTL |
| | | | | MNWVRQAPGKG | DSDDGNTYLD | QGALVTVSSASTKGPSVFPLAPSSKSTSGGTAALGCLV | SYRASGVPDRFSGSGSGTD |
| | | | | LEWVSSISSSS | WYLQKPGQSP | KDYFPEPVTVSWNSGALTSGVHTFPAVLQSSGLYSLSS | FTLKISRVEAEDVGIYYCM |
| | | | | SYIDFAESVKG | QLLIYTLSYR | VVTVPSSSLGTQTYICNVNHKPSNTKVDKKVEPKSCDK | QRIEFPITFGQGTKVEIKR |
| | | | | RFTISRDNAKN | ASGVPDRFSG | THTCPPCPAPELLGGPSVFLFPPKPKDTLMISRTPEVT | TVAAPSVFIFPPSDEQLKS |
| | | | | SLYLQMISLRA | SGSGTDFTLK | CVVVDVSHEDPEVKFNWYVDGVEVHNAKTKPREEQYNS | GTASVVCLLNNFYPREAKV |
| | | | | EDTAVYYCARD | ISRVEAEDVG | TYRVVSVLTVLHQDWLNGKEYKCKVSNKALPAPIEKTI | QWKVDNALQSGNSQESVTE |
| | | | | GDIVSTIRGSF | IYYCMQRIEF | SKAKGQPREPQVYTLPPSREEMTKNQVSLTCLVKGFYP | QDSKDSTYSLSSTLTLSKA |
| | | | | DYWGQGALVTV | PITFGQGTKV | SDIAVEWESNGQPENNYKTTPPVLDSDGSFFLYSKLTV | DYEKHKVYACEVTHQGLSS |
| | | | | SS | EIK | DKSRWQQGNVFSCSVMHEALHNHYTQKSLSLSPGK | PVTKSFNRGEC |
| | | | | 1493 | 1494 | 1495 | 1496 |
| 45 | VG9B137 | IgG1 | Kappa | EVQLVESGGGL | EIVMTQSPGT | EVQLVESGGGLVKPGGSLRLSCAAFGITFSSYSMDWVR | EIVMTQSPGTLSLSPGERA |
| | | | | VKPGGSLRLSC | LSLSPGERAT | QAPGRGLEWVSSIGSSSSYIFYADSVKGRFTISRDNAK | TLSCRASQSVSSSYLAWYQ |
| | | | | AAFGITFSSYS | LSCRASQSVS | NSLYLQMNSLRAEDTAVYYCATSYSWNYGGAFDIWGQG | QKPGQAPRLLIYGASSRAT |
| | | | | MDWVRQAPGRG | SSYLAWYQQK | TMVTVSSASTKGPSVFPLAPSSKSTSGGTAALGCLVKD | GIPDRFSGSGSGTDFTLTI |
| | | | | LEWVSSIGSSS | PGQAPRLLIY | YFPEPVTVSWNSGALTSGVHTFPAVLQSSGLYSLSSVV | SRLEPEDFAVYFCQQYGSS |
| | | | | SYIFYADSVKG | GASSRATGIP | TVPSSSLGTQTYICNVNHKPSNTKVDKKVEPKSCDKTH | PPYTFGQGTKVEIKRTVAA |
| | | | | RFTISRDNAKN | DRFSGSGSGT | TCPPCPAPELLGGPSVFLFPPKPKDTLMISRTPEVTCV | PSVFIFPPSDEQLKSGTAS |
| | | | | SLYLQMNSLRA | DFTLTISRLE | VVDVSHEDPEVKFNWYVDGVEVHNAKTKPREEQYNSTY | VVCLLNNFYPREAKVQWKV |
| | | | | EDTAVYYCATS | PEDFAVYFCQ | RVVSVLTVLHQDWLNGKEYKCKVSNKALPAPIEKTISK | DNALQSGNSQESVTEQDSK |
| | | | | YSWNYGGAFDI | QYGSSPPYTF | AKGQPREPQVYTLPPSREEMTKNQVSLTCLVKGFYPSD | DSTYSLSSTLTLSKADYEK |
| | | | | WGQGTMVTVSS | GQGTKVEIK | IAVEWESNGQPENNYKTTPPVLDSDGSFFLYSKLTVDK | HKVYACEVTHQGLSSPVTK |
| | | | | | | SRWQQGNVFSCSVMHEALHNHYTQKSLSLSPGK | SFNRGEC |
| | | | | 1527 | 1528 | 1529 | 1530 |
| 46 | VG9B33 | IgG1 | Kappa | EVQLVESGGGL | DIVMTQSPSS | EVQLVESGGGLVKPGGSLRLSCAASGFTFSSYNMNWVR | DIVMTQSPSSLSASVGDRV |
| | | | | VKPGGSLRLSC | LSASVGDRVT | QAPGKGLEWVSSISTSSSYIYYADSVKGRFTISRDNAK | TITCRASQDINNYLAWFQQ |
| | | | | AASGFTFSSYN | ITCRASQDIN | NSLYLQMNGLRAEDTAVYYCARDTSVTKYPDTFDIWGQ | KPGKVPNLLIYGASTLQSG |
| | | | | MNWVRQAPGKG | NYLAWFQQKP | GTMVTVSSASTKGPSVFPLAPSSKSTSGGTAALGCLVK | VPSRFTGSGSGTDFTLTIS |
| | | | | LEWVSSISTSS | GKVPNLLIYG | DYFPEPVTVSWNSGALTSGVHTFPAVLQSSGLYSLSSV | SLQPEDVATYYCQKYNSAP |
| | | | | SYIYYADSVKG | ASTLQSGVPS | VTVPSSSLGTQTYICNVNHKPSNTKVDKKVEPKSCDKT | FTFGPGTKVDIKRTVAAPS |
| | | | | RFTISRDNAKN | RFTGSGSGTD | HTCPPCPAPELLGGPSVFLFPPKPKDTLMISRTPEVTC | VFIFPPSDEQLKSGTASVV |
| | | | | SLYLQMNGLRA | FTLTISSLQP | VVVDVSHEDPEVKFNWYVDGVEVHNAKTKPREEQYNST | CLLNNFYPREAKVQWKVDN |
| | | | | EDTAVYYCARD | EDVATYYCQK | YRVVSVLTVLHQDWLNGKEYKCKVSNKALPAPIEKTIS | ALQSGNSQESVTEQDSKDS |
| | | | | TSVTKYPDTFD | YNSAPFTFGP | KAKGQPREPQVYTLPPSREEMTKNQVSLTCLVKGFYPS | TYSLSSTLTLSKADYEKHK |
| | | | | IWGQGTMVTVS | GTKVDIK | DIAVEWESNGQPENNYKTTPPVLDSDGSFFLYSKLTVD | VYACEVTHQGLSSPVTKSF |
| | | | | S | | KSRWQQGNVFSCSVMHEALHNHYTQKSLSLSPGK | NRGEC |
| | | | | 1561 | 1562 | 1563 | 1564 |
| 47 | VG9B162 | IgG1 | Lambda | QVQLVESGAEV | QLVLTQSSSA | QVQLVESGAEVTKPGASVKVSCKASGYTFTGDYMHWVR | QLVLTQSSSASGSPGQSVT |
| | | | | TKPGASVKVSC | SGSPGQSVTI | QAPGQGLEWMGWINPNSGYTNYAQKFQGRVTMTRDTSI | ISCTGTSSDVGGYNYVSWY |
| | | | | KASGYTFTGDY | SCTGTSSDVG | STAYMELSRLRSDDTAVYYCAREGDAFDVWGQGTMVTV | QQHPGKAPKLMIYEVSKRP |
| | | | | MHWVRQAPGQG | GYNYVSWYQQ | SSASTKGPSVFPLAPSSKSTSGGTAALGCLVKDYFPEP | SGVPDRFSGSKSGNTASLT |
| | | | | LEWMGWINPNS | HPGKAPKLMI | VTVSWNSGALTSGVHTFPAVLQSSGLYSLSSVVTVPSS | VSGLQAEDEADYYCNSYAG |
| | | | | GYTNYAQKFQG | YEVSKRPSGV | SLGTQTYICNVNHKPSNTKVDKKVEPKSCDKTHTCPPC | SNNFEVFGGGTKLTVLGQP |
| | | | | RVTMTRDTSIS | PDRFSGSKSG | PAPELLGGPSVFLFPPKPKDTLMISRTPEVTCVVVDVS | KAAPSVTLFPPSSEELQAN |
| | | | | TAYMELSRLRS | NTASLTVSGL | HEDPEVKFNWYVDGVEVHNAKTKPREEQYNSTYRVVSV | KATLVCLISDFYPGAVTVA |
| | | | | DDTAVYYCARE | QAEDEADYYC | LTVLHQDWLNGKEYKCKVSNKALPAPIEKTISKAKGQP | WKADSSPVKAGVETTTPSK |
| | | | | GDAFDVWGQGT | NSYAGSNNFE | REPQVYTLPPSREEMTKNQVSLTCLVKGFYPSDIAVEW | QSNNKYAASSYLSLTPEQW |
| | | | | MVTVSS | VFGGGTKLTV | ESNGQPENNYKTTPPVLDSDGSFFLYSKLTVDKSRWQQ | KSHRSYSCQVTHEGSTVEK |
| | | | | | L | GNVFSCSVMHEALHNHYTQKSLSLSPGK | TVAPTECS |
| | | | | 1595 | 1596 | 1597 | 1598 |
| 48 | VG9B152 | IgG1 | Lambda | EVQLVESGGGL | SYELMQPPSV | EVQLVESGGGLVRPGGSLRLSCEASGFTFSSYSMIWVR | SYELMQPPSVSVSPGQTAS |
| | | | | VRPGGSLRLSC | SVSPGQTASI | QAPGKGLEWVSSISSSSDYIYNADSVKGRFTISRDNAK | ITCSGDKLGDKYACWYQQK |
| | | | | EASGFTFSSYS | TCSGDKLGDK | NSLYLQMNSLRAEDTAVYYCARDWELLGFDCWGQGTTV | PGQSPVLVIYQHNKRPSGI |
| | | | | MIWVRQAPGKG | YACWYQQKPG | TVSSASTKGPSVFPLAPSSKSTSGGTAALGCLVKDYFP | PERFSGSNSGKTATLTISG |
| | | | | LEWVSSISSSS | QSPVLVIYQH | EPVTVSWNSGALTSGVHTFPAVLQSSGLYSLSSVVTVP | TQAMDEADYYCQAWDSTTV |
| | | | | DYIYNADSVKG | NKRPSGIPER | SSSLGTQTYICNVNHKPSNTKVDKKVEPKSCDKTHTCP | VFGGGTKLTVLGQPKAAPS |
| | | | | RFTISRDNAKN | FSGSNSGKTA | PCPAPELLGGPSVFLFPPKPKDTLMISRTPEVTCVVVD | VTLFPPSSEELQANKATLV |
| | | | | SLYLQMNSLRA | TLTISGTQAM | VSHEDPEVKFNWYVDGVEVHNAKTKPREEQYNSTYRVV | CLISDFYPGAVTVAWKADS |
| | | | | EDTAVYYCARD | DEADYYCQAW | SVLTVLHQDWLNGKEYKCKVSNKALPAPIEKTISKAKG | SPVKAGVETTTPSKQSNNK |
| | | | | WELLGFDCWGQ | DSTTVVFGGG | QPREPQVYTLPPSREEMTKNQVSLTCLVKGFYPSDIAV | YAASSYLSLTPEQWKSHRS |
| | | | | GTTVTVSS | TKLTVL | EWESNGQPENNYKTTPPVLDSDGSFFLYSKLTVDKSRW | YSCQVTHEGSTVEKTVAPT |
| | | | | | | QQGNVFSCSVMHEALHNHYTQKSLSLSPGK | ECS |
| | | | | 1629 | 1630 | 1631 | 1632 |
| 49 | VG9B64 | IgG1 | Kappa | QVQLVESGPTL | DIVMTQTPLS | QVQLVESGPTLVKPTQTLTLTCTFSGFSLSISGVSVGW | DIVMTQTPLSSPVTLGQPA |
| | | | | VKPTQTLTLTC | SPVTLGQPAS | IRQPPGKALEWLALIYWNDDKRYSPSLQSRVTITKDTS | SISCRSSESLVHSDGNTYL |
| | | | | TFSGFSLSISG | ISCRSSESLV | KNQVVLTVTNMDSVDTATYYCVHSGQWLEGDAFDIWGQ | SWLQQSPGQPPRLLIYKIS |
| | | | | VSVGWIRQPPG | HSDGNTYLSW | GTMVTVSSASTKGPSVFPLAPSSKSTSGGTAALGCLVK | NRFSGVPDRFSGSGAGTDF |
| | | | | KALEWLALIYW | LQQSPGQPPR | DYFPEPVTVSWNSGALTSGVHTFPAVLQSSGLYSLSSV | TLKISRVEAEDVGVYYCMQ |
| | | | | NDDKRYSPSLQ | LLIYKISNRF | VTVPSSSLGTQTYICNVNHKPSNTKVDKKVEPKSCDKT | ATQFPLTFGGGTKLEIKRT |
| | | | | SRVTITKDTSK | SGVPDRFSGS | HTCPPCPAPELLGGPSVFLFPPKPKDTLMISRTPEVTC | VAAPSVFIFPPSDEQLKSG |
| | | | | NQVVLTVTNMD | GAGTDFTLKI | VVVDVSHEDPEVKFNWYVDGVEVHNAKTKPREEQYNST | TASVVCLLNNFYPREAKVQ |
| | | | | SVDTATYYCVH | SRVEAEDVGV | YRVVSVLTVLHQDWLNGKEYKCKVSNKALPAPIEKTIS | WKVDNALQSGNSQESVTEQ |
| | | | | SGQWLEGDAFD | YYCMQATQFP | KAKGQPREPQVYTLPPSREEMTKNQVSLTCLVKGFYPS | DSKDSTYSLSSTLTLSKAD |
| | | | | IWGQGTMVTVS | LTFGGGTKLE | DIAVEWESNGQPENNYKTTPPVLDSDGSFFLYSKLTVD | YEKHKVYACEVTHQGLSSP |
| | | | | S | IK | KSRWQQGNVFSCSVMHEALHNHYTQKSLSLSPGK | VTKSFNRGEC |
| | | | | 1663 | 1664 | 1665 | 1666 |
| 50 | VG9B21 | IgG1 | Kappa | EVQLLESGGGL | DIVMTQSPSS | EVQLLESGGGLVKPGGSLRLSCAASGFSFSNYDMNWVR | DIVMTQSPSSLSASVGDRV |
| | | | | VKPGGSLRLSC | LSASVGDRVT | QAPRKGLEWVSSISSSSHYIYYADSLKGRFTISRDNAK | TITCRASQGIYNYLAWYQQ |
| | | | | AASGFSFSNYD | ITCRASQGIY | NSLFLQMNSLRAEDTAVYYCARDRGVTTDYYYYALDVW | KPGNVPKLLIYAASTLQSG |
| | | | | MNWVRQAPRKG | NYLAWYQQKP | GQGTMVTVSSASTKGPSVFPLAPSSKSTSGGTAALGCL | VPSRFSGSGSGTDFSLTIS |
| | | | | LEWVSSISSSS | GNVPKLLIYA | VKDYFPEPVTVSWNSGALTSGVHTFPAVLQSSGLYSLS | SLQPEDFTTYYCQKYNRAP |
| | | | | HYIYYADSLKG | ASTLQSGVPS | SVVTVPSSSLGTQTYICNVNHKPSNTKVDKKVEPKSCD | FTFGPGTKVDIKRTVAAPS |
| | | | | RFTISRDNAKN | RFSGSGSGTD | KTHTCPPCPAPELLGGPSVFLFPPKPKDTLMISRTPEV | VFIFPPSDEQLKSGTASVV |
| | | | | SLFLQMNSLRA | FSLTISSLQP | TCVVVDVSHEDPEVKFNWYVDGVEVHNAKTKPREEQYN | CLLNNFYPREAKVQWKVDN |
| | | | | EDTAVYYCARD | EDFTTYYCQK | STYRVVSVLTVLHQDWLNGKEYKCKVSNKALPAPIEKT | ALQSGNSQESVTEQDSKDS |
| | | | | RGVTTDYYYYA | YNRAPFTFGP | ISKAKGQPREPQVYTLPPSREEMTKNQVSLTCLVKGFY | TYSLSSTLTLSKADYEKHK |
| | | | | LDVWGQGTMVT | GTKVDIK | PSDIAVEWESNGQPENNYKTTPPVLDSDGSFFLYSKLT | VYACEVTHQGLSSPVTKSF |
| | | | | VSS | | VDKSRWQQGNVFSCSVMHEALHNHYTQKSLSLSPGK | NRGEC |
| | | | | 1697 | 1698 | 1699 | 1700 |
| 51 | VG9B128 | IgG1 | Kappa | EVQLVESGGGV | DIVMTQTPAT | EVQLVESGGGVVQPGKSLRLSCTASGFTFGTYGMHWVR | DIVMTQTPATLSLSPGERA |
| | | | | VQPGKSLRLSC | LSLSPGERAT | QAPGKGLDWVAVIWYNGSNKYYADSVKGRFTISRDNSK | TLSCRASQSVIDYLAWFQQ |
| | | | | TASGFTFGTYG | LSCRASQSVI | NTLYLQMNSLRTEDTAVYYCARGGFGESFDSWGQGTLV | KPGQAPRLLIYDASNRATG |
| | | | | MHWVRQAPGKG | DYLAWFQQKP | TVSSASTKGPSVFPLAPSSKSTSGGTAALGCLVKDYFP | IPARFSGSGSGTDFTLTIS |
| | | | | LDWVAVIWYNG | GQAPRLLIYD | EPVTVSWNSGALTSGVHTFPAVLQSSGLYSLSSVVTVP | SLEPEDFAVYYCQQRSNWP |
| | | | | SNKYYADSVKG | ASNRATGIPA | SSSLGTQTYICNVNHKPSNTKVDKKVEPKSCDKTHTCP | LTFGGGTKVEIKRTVAAPS |
| | | | | RFTISRDNSKN | RFSGSGSGTD | PCPAPELLGGPSVFLFPPKPKDTLMISRTPEVTCVVVD | VFIFPPSDEQLKSGTASVV |
| | | | | TLYLQMNSLRT | FTLTISSLEP | VSHEDPEVKFNWYVDGVEVHNAKTKPREEQYNSTYRVV | CLLNNFYPREAKVQWKVDN |
| | | | | EDTAVYYCARG | EDFAVYYCQQ | SVLTVLHQDWLNGKEYKCKVSNKALPAPIEKTISKAKG | ALQSGNSQESVTEQDSKDS |
| | | | | GFGESFDSWGQ | RSNWPLTFGG | QPREPQVYTLPPSREEMTKNQVSLTCLVKGFYPSDIAV | TYSLSSTLTLSKADYEKHK |
| | | | | GTLVTVSS | GTKVEIK | EWESNGQPENNYKTTPPVLDSDGSFFLYSKLTVDKSRW | VYACEVTHQGLSSPVTKSF |
| | | | | | | QQGNVFSCSVMHEALHNHYTQKSLSLSPGK | NRGEC |
| | | | | 1731 | 1732 | 1733 | 1734 |
| 52 | VG9B66 | IgG1 | Kappa | EVQLVESGGGL | EIVLTQSPSS | EVQLVESGGGLVKPGGSLRLSCAASGFTFSTYTMNWVR | EIVLTQSPSSLSTSVGDRV |
| | | | | VKPGGSLRLSC | LSTSVGDRVT | QAPGKGLEWVSSISSSSFYMDYADSVKGRFTISRDNAK | TITCRASQDITNFLAWYQQ |
| | | | | AASGFTFSTYT | ITCRASQDIT | NSLSLLMNSLRAEDTAVYYCARDGDIVATIRGSFDYWG | KPGKLPKLLIYTASTLQSG |
| | | | | MNWVRQAPGKG | NFLAWYQQKP | QGTMVTVSSASTKGPSVFPLAPSSKSTSGGTAALGCLV | VPSRFSGSASGPDFTLTIS |
| | | | | LEWVSSISSSS | GKLPKLLIYT | KDYFPEPVTVSWNSGALTSGVHTFPAVLQSSGLYSLSS | SLQPEDVATYYCQKYNSAP |
| | | | | FYMDYADSVKG | ASTLQSGVPS | VVTVPSSSLGTQTYICNVNHKPSNTKVDKKVEPKSCDK | FTFGPGTKVEIKRTVAAPS |
| | | | | RFTISRDNAKN | RFSGSASGPD | THTCPPCPAPELLGGPSVFLFPPKPKDTLMISRTPEVT | VFIFPPSDEQLKSGTASVV |
| | | | | SLSLLMNSLRA | FTLTISSLQP | CVVVDVSHEDPEVKFNWYVDGVEVHNAKTKPREEQYNS | CLLNNFYPREAKVQWKVDN |
| | | | | EDTAVYYCARD | EDVATYYCQK | TYRVVSVLTVLHQDWLNGKEYKCKVSNKALPAPIEKTI | ALQSGNSQESVTEQDSKDS |
| | | | | GDIVATIRGSF | YNSAPFTFGP | SKAKGQPREPQVYTLPPSREEMTKNQVSLTCLVKGFYP | TYSLSSTLTLSKADYEKHK |
| | | | | DYWGQGTMVTV | GTKVEIK | SDIAVEWESNGQPENNYKTTPPVLDSDGSFFLYSKLTV | VYACEVTHQGLSSPVTKSF |
| | | | | SS | | DKSRWQQGNVFSCSVMHEALHNHYTQKSLSLSPGK | NRGEC |
| | | | | 1765 | 1766 | 1767 | 1768 |
| 53 | VG9B32 | IgG1 | Kappa | EVQLVQSGGDL | DIVMTQTPLS | EVQLVQSGGDLLQPGGSLRLSCAASGFTFSRYAMNWVR | DIVMTQTPLSSPVTLGQPA |
| | | | | LQPGGSLRLSC | SPVTLGQPAS | QAPGKGLEWVSFISGTGYTVYYADSVKGRFTISRDNAK | SISCRSGQSLVHSDGNTYL |
| | | | | AASGFTFSRYA | ISCRSGQSLV | NSLYLQMNSLRDEDTAVYYCARDQEPGFDYWGQGTLVT | SWLQQRPGQPPRLLIYKIS |
| | | | | MNWVRQAPGKG | HSDGNTYLSW | VSSASTKGPSVFPLAPSSKSTSGGTAALGCLVKDYFPE | NRFSGVPDRFSGSGAGTDF |
| | | | | LEWVSFISGTG | LQQRPGQPPR | PVTVSWNSGALTSGVHTFPAVLQSSGLYSLSSVVTVPS | TLKISRVEAEDVGVYYCMQ |
| | | | | YTVYYADSVKG | LLIYKISNRF | SSLGTQTYICNVNHKPSNTKVDKKVEPKSCDKTHTCPP | ATHFPFTFGQGTKLEIKRT |
| | | | | RFTISRDNAKN | SGVPDRFSGS | CPAPELLGGPSVFLFPPKPKDTLMISRTPEVTCVVVDV | VAAPSVFIFPPSDEQLKSG |
| | | | | SLYLQMNSLRD | GAGTDFTLKI | SHEDPEVKFNWYVDGVEVHNAKTKPREEQYNSTYRVVS | TASVVCLLNNFYPREAKVQ |
| | | | | EDTAVYYCARD | SRVEAEDVGV | VLTVLHQDWLNGKEYKCKVSNKALPAPIEKTISKAKGQ | WKVDNALQSGNSQESVTEQ |
| | | | | QEPGFDYWGQG | YYCMQATHFP | PREPQVYTLPPSREEMTKNQVSLTCLVKGFYPSDIAVE | DSKDSTYSLSSTLTLSKAD |
| | | | | TLVTVSS | FTFGQGTKLE | WESNGQPENNYKTTPPVLDSDGSFFLYSKLTVDKSRWQ | YEKHKVYACEVTHQGLSSP |
| | | | | | IK | QGNVFSCSVMHEALHNHYTQKSLSLSPGK | VTKSFNRGEC |
| | | | | 1799 | 1800 | 1801 | 1802 |
| 54 | VG9B57 | IgG1 | Kappa | EVQLVESGGGL | DIQMTQSPSA | EVQLVESGGGLVKPGGSLRLSCAASGFTFSSHDMNWVR | DIQMTQSPSAMSASVGDRV |
| | | | | VKPGGSLRLSC | MSASVGDRVT | QAPGKGLGWVSSISSSSSYIFYADSVKGRFTISRDNAK | TITCRASQDITNYLAWFQQ |
| | | | | AASGFTFSSHD | ITCRASQDIT | NSLYLQMSSLRAEDTAVYYCARDLGVGVRDYYYYGMDV | KPGKVPKRLIYAASSLQGG |
| | | | | MNWVRQAPGKG | NYLAWFQQKP | WGQGTMVTVSSASTKGPSVFPLAPSSKSTSGGTAALGC | VPSRFSGSGSGTEFTLTIS |
| | | | | LGWVSSISSSS | GKVPKRLIYA | LVKDYFPEPVTVSWNSGALTSGVHTFPAVLQSSGLYSL | SLQPEDFSTYYCLQHDTYP |
| | | | | SYIFYADSVKG | ASSLQGGVPS | SSVVTVPSSSLGTQTYICNVNHKPSNTKVDKKVEPKSC | YTFGQGTKLEIKRTVAAPS |
| | | | | RFTISRDNAKN | RFSGSGSGTE | DKTHTCPPCPAPELLGGPSVFLFPPKPKDTLMISRTPE | VFIFPPSDEQLKSGTASVV |
| | | | | SLYLQMSSLRA | FTLTISSLQP | VTCVVVDVSHEDPEVKFNWYVDGVEVHNAKTKPREEQY | CLLNNFYPREAKVQWKVDN |
| | | | | EDTAVYYCARD | EDFSTYYCLQ | NSTYRVVSVLTVLHQDWLNGKEYKCKVSNKALPAPIEK | ALQSGNSQESVTEQDSKDS |
| | | | | LGVGVRDYYYY | HDTYPYTFGQ | TISKAKGQPREPQVYTLPPSREEMTKNQVSLTCLVKGF | TYSLSSTLTLSKADYEKHK |
| | | | | GMDVWGQGTMV | GTKLEIK | YPSDIAVEWESNGQPENNYKTTPPVLDSDGSFFLYSKL | VYACEVTHQGLSSPVTKSF |
| | | | | TVSS | | TVDKSRWQQGNVFSCSVMHEALHNHYTQKSLSLSPGK | NRGEC |
| | | | | 1833 | 1834 | 1835 | 1836 |
| 55 | VG9B135 | IgG1 | Kappa | QVTLKESGPTL | DIVMTQSPGT | QVTLKESGPTLVTPSQTLTLTCTFSGFSLTTYGVGVGW | DIVMTQSPGTLSLSPGERA |
| | | | | VTPSQTLTLTC | LSLSPGERAT | IRQPPGKALEWLALIYWNDDKRYYPSLNNRLTITKDTS | TLSCRASQSVSSSYLAWYQ |
| | | | | TFSGFSLTTYG | LSCRASQSVS | KHLVVLTLTNMDPVDTATYYCAHDYDFWSGYFDYWGQG | QKPGQAPRLLIYGASSRAT |
| | | | | VGVGWIRQPPG | SSYLAWYQQK | TLVTVSSASTKGPSVFPLAPSSKSTSGGTAALGCLVKD | GIPDRFSGSGSGTDFTLTI |
| | | | | KALEWLALIYW | PGQAPRLLIY | YFPEPVTVSWNSGALTSGVHTFPAVLQSSGLYSLSSW | SRLEPEDFAVYYCQQYGSS |
| | | | | NDDKRYYPSLN | GASSRATGIP | TVPSSSLGTQTYICNVNHKPSNTKVDKKVEPKSCDKTH | PLTFGGGTKVEIKRTVAAP |
| | | | | NRLTITKDTSK | DRFSGSGSGT | TCPPCPAPELLGGPSVFLFPPKPKDTLMISRTPEVTCV | SVFIFPPSDEQLKSGTASV |
| | | | | HLVVLTLTNMD | DFTLTISRLE | VVDVSHEDPEVKFNWYVDGVEVHNAKTKPREEQYNSTY | VCLLNNFYPREAKVQWKVD |
| | | | | PVDTATYYCAH | PEDFAVYYCQ | RVVSVLTVLHQDWLNGKEYKCKVSNKALPAPIEKTISK | NALQSGNSQESVTEQDSKD |
| | | | | DYDFWSGYFDY | QYGSSPLTFG | AKGQPREPQVYTLPPSREEMTKNQVSLTCLVKGFYPSD | STYSLSSTLTLSKADYEKH |
| | | | | WGQGTLVTVSS | GGTKVEIK | IAVEWESNGQPENNYKTTPPVLDSDGSFFLYSKLTVDK | KVYACEVTHQGLSSPVTKS |
| | | | | | | SRWQQGNVFSCSVMHEALHNHYTQKSLSLSPGK | FNRGEC |
| | | | | 1867 | 1868 | 1869 | 1870 |
| 56 | VG9B60 | IgG1 | Kappa | QVQLVQSGAEV | DIVMTQSPGT | QVQLVQSGAEVKKPGASVKVSCKASGYTFTGYYMHWVR | DIVMTQSPGTLSLSPGERA |
| | | | | KKPGASVKVSC | LSLSPGERAT | QAPGQGLEWMGRINPNSGVTHYAQKFQGRVTMTRDTSI | TLSCRASQSFSGSYLAWYQ |
| | | | | KASGYTFTGYY | LSCRASQSFS | STAYMELSRLRSDDTAVYYCARGGSLVRGVISGLDYWG | QKPGQAPSLLIYGASSRAT |
| | | | | MHWVRQAPGQG | GSYLAWYQQK | QGTTVTVSSASTKGPSVFPLAPSSKSTSGGTAALGCLV | GIPDRFSGSGSGTDFTLTI |
| | | | | LEWMGRINPNS | PGQAPSLLIY | KDYFPEPVTVSWNSGALTSGVHTFPAVLQSSGLYSLSS | SRLEPEDFALYYCQQYGSS |
| | | | | GVTHYAQKFQG | GASSRATGIP | VVTVPSSSLGTQTYICNVNHKPSNTKVDKKVEPKSCDK | PPYTFGQGTKVEIKRTVAA |
| | | | | RVTMTRDTSIS | DRFSGSGSGT | THTCPPCPAPELLGGPSVFLFPPKPKDTLMISRTPEVT | PSVFIFPPSDEQLKSGTAS |
| | | | | TAYMELSRLRS | DFTLTISRLE | CVVVDVSHEDPEVKFNWYVDGVEVHNAKTKPREEQYNS | VVCLLNNFYPREAKVQWKV |
| | | | | DDTAVYYCARG | PEDFALYYCQ | TYRVVSVLTVLHQDWLNGKEYKCKVSNKALPAPIEKTI | DNALQSGNSQESVTEQDSK |
| | | | | GSLVRGVISGL | QYGSSPPYTF | SKAKGQPREPQVYTLPPSREEMTKNQVSLTCLVKGFYP | DSTYSLSSTLTLSKADYEK |
| | | | | DYWGQGTTVTV | GQGTKVEIK | SDIAVEWESNGQPENNYKTTPPVLDSDGSFFLYSKLTV | HKVYACEVTHQGLSSPVTK |
| | | | | SS | | DKSRWQQGNVFSCSVMHEALHNHYTQKSLSLSPGK | SFNRGEC |
| | | | | 1901 | 1902 | 1903 | 1904 |
| 57 | VG9B409 | IgG1 | Kappa | EVQLVESGAEV | EIVLTQSPGT | EVQLVESGAEVKKPGASVKVSCKASGYTLTSYYMHWVR | EIVLTQSPGTLSLSPGERA |
| | | | | KKPGASVKVSC | LSLSPGERAT | QAPGQGLEWMGIINPSGGSTSYAQKFQGRVTMTRDTST | TLSCRASQSVTSSYLAWYQ |
| | | | | KASGYTLTSYY | LSCRASQSVT | STVYMELSSLRSEDTAVYYCARGSYGWYFDLWGRGTLV | QKPGQTPRLLIYGASSRAT |
| | | | | MHWVRQAPGQG | SSYLAWYQQK | TVSSASTKGPSVFPLAPSSKSTSGGTAALGCLVKDYFP | GIPDRISGSGSGTDFTLTV |
| | | | | LEWMGIINPSG | PGQTPRLLIY | EPVTVSWNSGALTSGVHTFPAVLQSSGLYSLSSVVTVP | SRLEPEDFAVYYCQQYGSS |
| | | | | GSTSYAQKFQG | GASSRATGIP | SSSLGTQTYICNVNHKPSNTKVDKKVEPKSCDKTHTCP | PPYTFGQGTKLEIKRTVAA |
| | | | | RVTMTRDTSTS | DRISGSGSGT | PCPAPELLGGPSVFLFPPKPKDTLMISRTPEVTCVVVD | PSVFIFPPSDEQLKSGTAS |
| | | | | TVYMELSSLRS | DFTLTVSRLE | VSHEDPEVKFNWYVDGVEVHNAKTKPREEQYNSTYRVV | VVCLLNNFYPREAKVQWKV |
| | | | | EDTAVYYCARG | PEDFAVYYCQ | SVLTVLHQDWLNGKEYKCKVSNKALPAPIEKTISKAKG | DNALQSGNSQESVTEQDSK |
| | | | | SYGWYFDLWGR | QYGSSPPYTF | QPREPQVYTLPPSREEMTKNQVSLTCLVKGFYPSDIAV | DSTYSLSSTLTLSKADYEK |
| | | | | GTLVTVSS | GQGTKLEIK | EWESNGQPENNYKTTPPVLDSDGSFFLYSKLTVDKSRW | HKVYACEVTHQGLSSPVTK |
| | | | | | | QQGNVFSCSVMHEALHNHYTQKSLSLSPGK | SFNRGEC |
| | | | | 1935 | 1936 | 1937 | 1938 |
| 58 | VG9B411 | IgG1 | Kappa | EVQLVESGGGL | EIVMTQSPSS | EVQLVESGGGLVKPGGSLRLSCSASGFTFSNYDMNWVR | EIVMTQSPSSVSASVGDRV |
| | | | | VKPGGSLRLSC | VSASVGDRVT | QAPGKGLEWVSSISSSSSYIYYADSVKGRFTISRDNAK | TITCRASQGINSWLAWYQQ |
| | | | | SASGFTFSNYD | ITCRASQGIN | NSLYLQMNSLRAEDTAVYHCARDVGVTTDYYYYGMDVW | DPYKAPKLLIYAASTLQSG |
| | | | | MNWVRQAPGKG | SWLAWYQQDP | GQGTTVTVSSASTKGPSVFPLAPSSKSTSGGTAALGCL | VPSRFGGRGSGTEFTLTIS |
| | | | | LEWVSSISSSS | YKAPKLLIYA | VKDYFPEPVTVSWNSGALTSGVHTFPAVLQSSGLYSLS | SLQPEDFAVYYCQQYNNWP |
| | | | | SYIYYADSVKG | ASTLQSGVPS | SVVTVPSSSLGTQTYICNVNHKPSNTKVDKKVEPKSCD | RTFGQGTKVDIKRTVAAPS |
| | | | | RFTISRDNAKN | RFGGRGSGTE | KTHTCPPCPAPELLGGPSVFLFPPKPKDTLMISRTPEV | VFIFPPSDEQLKSGTASVV |
| | | | | SLYLQMNSLRA | FTLTISSLQP | TCVVVDVSHEDPEVKFNWYVDGVEVHNAKTKPREEQYN | CLLNNFYPREAKVQWKVDN |
| | | | | EDTAVYHCARD | EDFAVYYCQQ | STYRVVSVLTVLHQDWLNGKEYKCKVSNKALPAPIEKT | ALQSGNSQESVTEQDSKDS |
| | | | | VGVTTDYYYYG | YNNWPRTFGQ | ISKAKGQPREPQVYTLPPSREEMTKNQVSLTCLVKGFY | TYSLSSTLTLSKADYEKHK |
| | | | | MDVWGQGTTVT | GTKVDIK | PSDIAVEWESNGQPENNYKTTPPVLDSDGSFFLYSKLT | VYACEVTHQGLSSPVTKSF |
| | | | | VSS | | VDKSRWQQGNVFSCSVMHEALHNHYTQKSLSLSPGK | NRGEC |
| | | | | 1969 | 1970 | 1971 | 1972 |
| 59 | VG9B129 | IgG1 | Kappa | EVQLVQSGAEV | EIVLTQSPGT | EVQLVQSGAEVKKPGSSVKVSCKASGGTFSSYVISWVR | EIVLTQSPGTLSLSPGERA |
| | | | | KKPGSSVKVSC | LSLSPGERAT | QAPGQGLEWMGGILPILSTANYAQKFQGRVTITADKST | TLSCRASQSVSNNYLAWYQ |
| | | | | KASGGTFSSYV | LSCRASQSVS | STAYMELSSLRSEDTAVYYCARAHDYYYGMDVWGQGTT | QKPGQAPRLLIYGASSRAT |
| | | | | ISWVRQAPGQG | NNYLAWYQQK | VTVSSASTKGPSVFPLAPSSKSTSGGTAALGCLVKDYF | GIPDRFSGSGSGTDFTLTI |
| | | | | LEWMGGILPIL | PGQAPRLLIY | PEPVTVSWNSGALTSGVHTFPAVLQSSGLYSLSSVVTV | SRLEPEDFAVYYCQQYGSS |
| | | | | STANYAQKFQG | GASSRATGIP | PSSSLGTQTYICNVNHKPSNTKVDKKVEPKSCDKTHTC | PPYTFGQGTKVDIKRTVAA |
| | | | | RVTITADKSTS | DRFSGSGSGT | PPCPAPELLGGPSVFLFPPKPKDTLMISRTPEVTCVVV | PSVFIFPPSDEQLKSGTAS |
| | | | | TAYMELSSLRS | DFTLTISRLE | DVSHEDPEVKFNWYVDGVEVHNAKTKPREEQYNSTYRV | VVCLLNNFYPREAKVQWKV |
| | | | | EDTAVYYCARA | PEDFAVYYCQ | VSVLTVLHQDWLNGKEYKCKVSNKALPAPIEKTISKAK | DNALQSGNSQESVTEQDSK |
| | | | | HDYYYGMDVWG | QYGSSPPYTF | GQPREPQVYTLPPSREEMTKNQVSLTCLVKGFYPSDIA | DSTYSLSSTLTLSKADYEK |
| | | | | QGTTVTVSS | GQGTKVDIK | VEWESNGQPENNYKTTPPVLDSDGSFFLYSKLTVDKSR | HKVYACEVTHQGLSSPVTK |
| | | | | | | WQQGNVFSCSVMHEALHNHYTQKSLSLSPGK | SFNRGEC |
| | | | | 2003 | 2004 | 2005 | 2006 |
| 60 | VG9B396 | IgG1 | Lambda | EVQLVESGPGL | QSVLTQAPSV | EVQLVESGPGLVQPSETLSLTCTVSGDSIRGYYWNWIR | QSVLTQAPSVSGAPGQRVT |
| | | | | VQPSETLSLTC | SGAPGQRVTI | QPAGKGLEWIGRIFTTGNTNYNPSLKSRLTMSLDTSKN | ISCTGSSSNIGADYDVKWY |
| | | | | TVSGDSIRGYY | SCTGSSSNIG | QFSLKLNSVTAADTAVYYCAREKWDSSSSALYFDFWGQ | QQLPGTAPKLLIYGNTDRP |
| | | | | WNWIRQPAGKG | ADYDVKWYQQ | GTLVTVSSASTKGPSVFPLAPSSKSTSGGTAALGCLVK | SGVPDRFSGSKSGTSASLA |
| | | | | LEWIGRIFTTG | LPGTAPKLLI | DYFPEPVTVSWNSGALTSGVHTFPAVLQSSGLYSLSSV | ITGLQAEDEADYYCQSYDS |
| | | | | NTNYNPSLKSR | YGNTDRPSGV | VTVPSSSLGTQTYICNVNHKPSNTKVDKKVEPKSCDKT | RLTGYVVFGGGTKLTVLGQ |
| | | | | LTMSLDTSKNQ | PDRFSGSKSG | HTCPPCPAPELLGGPSVFLFPPKPKDTLMISRTPEVTC | PKAAPSVTLFPPSSEELQA |
| | | | | FSLKLNSVTAA | TSASLAITGL | VVVDVSHEDPEVKFNWYVDGVEVHNAKTKPREEQYNST | NKATLVCLISDFYPGAVTV |
| | | | | DTAVYYCAREK | QAEDEADYYC | YRVVSVLTVLHQDWLNGKEYKCKVSNKALPAPIEKTIS | AWKADSSPVKAGVETTTPS |
| | | | | WDSSSSALYFD | QSYDSRLTGY | KAKGQPREPQVYTLPPSREEMTKNQVSLTCLVKGFYPS | KQSNNKYAASSYLSLTPEQ |
| | | | | FWGQGTLVTVS | VVFGGGTKLT | DIAVEWESNGQPENNYKTTPPVLDSDGSFFLYSKLTVD | WKSHRSYSCQVTHEGSTVE |
| | | | | S | VL | KSRWQQGNVFSCSVMHEALHNHYTQKSLSLSPGK | KTVAPTECS |
| | | | | 2037 | 2038 | 2039 | 2040 |
| 61 | VG9B470 | IgG1 | Kappa | QVTLKESGPTL | EIVMTQSPSF | QVTLKESGPTLVKPTQTLTLTCTFSGFSLTTSGVGVGW | EIVMTQSPSFLSASVGDRV |
| | | | | VKPTQTLTLTC | LSASVGDRVT | IRQPPGKALEWLALILWNDHTIYSPSLKSRLTITKDTS | TFTCRASQGISRYLAWYQQ |
| | | | | TFSGFSLTTSG | FTCRASQGIS | KNQVVLTMTNMDPVDTAAYFCARDKWELRDAFDIWGQG | KPGRAPNLLIYVASTLQSG |
| | | | | VGVGWIRQPPG | RYLAWYQQKP | TMVTVSSASTKGPSVFPLAPSSKSTSGGTAALGCLVKD | VPSRFSGSGSGTEFTLTIS |
| | | | | KALEWLALILW | GRAPNLLIYV | YFPEPVTVSWNSGALTSGVHTFPAVLQSSGLYSLSSVV | SLLPEDFATYYCQQLISYP |
| | | | | NDHTIYSPSLK | ASTLQSGVPS | TVPSSSLGTQTYICNVNHKPSNTKVDKKVEPKSCDKTH | YTFSQGTRLEIKRTVAAPS |
| | | | | SRLTITKDTSK | RFSGSGSGTE | TCPPCPAPELLGGPSVFLFPPKPKDTLMISRTPEVTCV | VFIFPPSDEQLKSGTASVV |
| | | | | NQVVLTMTNMD | FTLTISSLLP | VVDVSHEDPEVKFNWYVDGVEVHNAKTKPREEQYNSTY | CLLNNFYPREAKVQWKVDN |
| | | | | PVDTAAYFCAR | EDFATYYCQQ | RVVSVLTVLHQDWLNGKEYKCKVSNKALPAPIEKTISK | ALQSGNSQESVTEQDSKDS |
| | | | | DKWELRDAFDI | LISYPYTFSQ | AKGQPREPQVYTLPPSREEMTKNQVSLTCLVKGFYPSD | TYSLSSTLTLSKADYEKHK |
| | | | | WGQGTMVTVSS | GTRLEIK | IAVEWESNGQPENNYKTTPPVLDSDGSFFLYSKLTVDK | VYACEVTHQGLSSPVTKSF |
| | | | | | | SRWQQGNVFSCSVMHEALHNHYTQKSLSLSPGK | NRGEC |
| | | | | 2071 | 2072 | 2073 | 2074 |
| 62 | VG9B111 | IgG1 | Kappa | QVQLLESGGGL | DIVMTQSPSS | QVQLLESGGGLVKPGGSLRLSCAASGFTFSTYSVNWVR | DIVMTQSPSSLSASVGDRV |
| | | | | VKPGGSLRLSC | LSASVGDRVT | QAPGKGLEWVSSISSDSSYIFYADSMKGRFTISRDNAK | TITCQASQDISHYLNWYQQ |
| | | | | AASGFTFSTYS | ITCQASQDIS | NSLYLHMNSLRAEDTAVYYCARDSVTGPFDYWGQGTLV | KPGKAPKLLIYDSYILETG |
| | | | | VNWVRQAPGKG | HYLNWYQQKP | TVSSASTKGPSVFPLAPSSKSTSGGTAALGCLVKDYFP | VTSRFSGSGSGTDFTFTIN |
| | | | | LEWVSSISSDS | GKAPKLLIYD | EPVTVSWNSGALTSGVHTFPAVLQSSGLYSLSSVVTVP | SLQPEDIAIYYCQQYDNLP |
| | | | | SYIFYADSMKG | SYILETGVTS | SSSLGTQTYICNVNHKPSNTKVDKKVEPKSCDKTHTCP | YTFGQGTKVEIKRTVAAPS |
| | | | | RFTISRDNAKN | RFSGSGSGTD | PCPAPELLGGPSVFLFPPKPKDTLMISRTPEVTCVVVD | VFIFPPSDEQLKSGTASVV |
| | | | | SLYLHMNSLRA | FTFTINSLQP | VSHEDPEVKFNWYVDGVEVHNAKTKPREEQYNSTYRVV | CLLNNFYPREAKVQWKVDN |
| | | | | EDTAVYYCARD | EDIAIYYCQQ | SVLTVLHQDWLNGKEYKCKVSNKALPAPIEKTISKAKG | ALQSGNSQESVTEQDSKDS |
| | | | | SVTGPFDYWGQ | YDNLPYTFGQ | QPREPQVYTLPPSREEMTKNQVSLTCLVKGFYPSDIAV | TYSLSSTLTLSKADYEKHK |
| | | | | GTLVTVSS | GTKVEIK | EWESNGQPENNYKTTPPVLDSDGSFFLYSKLTVDKSRW | VYACEVTHQGLSSPVTKSF |
| | | | | | | QQGNVFSCSVMHEALHNHYTQKSLSLSPGK | NRGEC |
| | | | | 2105 | 2106 | 2107 | 2108 |
| 63 | VG9B169 | IgG1 | Lambda | EVQLLESGPGL | QSALTQPPSA | EVQLLESGPGLVKPSETLSLTCSVSGGSITNYFWNWIR | QSALTQPPSASGTPGQRVT |
| | | | | VKPSETLSLTC | SGTPGQRVTI | QPPGKGLEWIGYIFYSGSTSYNPSLKSRVTISVDTSKN | ISCSGSSSNIGSNTVNWYQ |
| | | | | SVSGGSITNYF | SCSGSSSNIG | RFSLKLISVTAADTAVYYCARVGRWELRTAFDIWGQGT | QLPGTAPKLLIYSNNQRPS |
| | | | | WNWIRQPPGKG | SNTVNWYQQL | MVTVSSASTKGPSVFPLAPSSKSTSGGTAALGCLVKDY | GVPDRFSGSKSGTSASLAI |
| | | | | LEWIGYIFYSG | PGTAPKLLIY | FPEPVTVSWNSGALTSGVHTFPAVLQSSGLYSLSSVVT | SGLQSEDEADYYCAAWDDS |
| | | | | STSYNPSLKSR | SNNQRPSGVP | VPSSSLGTQTYICNVNHKPSNTKVDKKVEPKSCDKTHT | LNGPGVFGGGTKLTVLGQP |
| | | | | VTISVDTSKNR | DRFSGSKSGT | CPPCPAPELLGGPSVFLFPPKPKDTLMISRTPEVTCVV | KAAPSVTLFPPSSEELQAN |
| | | | | FSLKLISVTAA | SASLAISGLQ | VDVSHEDPEVKFNWYVDGVEVHNAKTKPREEQYNSTYR | KATLVCLISDFYPGAVTVA |
| | | | | DTAVYYCARVG | SEDEADYYCA | VVSVLTVLHQDWLNGKEYKCKVSNKALPAPIEKTISKA | WKADSSPVKAGVETTTPSK |
| | | | | RWELRTAFDIW | AWDDSLNGPG | KGQPREPQVYTLPPSREEMTKNQVSLTCLVKGFYPSDI | QSNNKYAASSYLSLTPEQW |
| | | | | GQGTMVTVSS | VFGGGTKLTV | AVEWESNGQPENNYKTTPPVLDSDGSFFLYSKLTVDKS | KSHRSYSCQVTHEGSTVEK |
| | | | | | L | RWQQGNVFSCSVMHEALHNHYTQKSLSLSPGK | TVAPTECS |
| | | | | 2139 | 2140 | 2141 | 2142 |
| 64 | VG9B639 | IgG1 | Kappa | QVQLVQSGGGL | DIQMTQSPSS | QVQLVQSGGGLVKPGGSLRLSCAASGFTFTTYRMNWVR | DIQMTQSPSSLSASVGDRV |
| | | | | VKPGGSLRLSC | LSASVGDRVT | QAPGKGLEWVSSISSSSIYIHSADSVKGRFTISRDNAK | TITCQASQDISHYLNWYQQ |
| | | | | AASGFTFTTYR | ITCQASQDIS | NSLYLQMNSLRAEDSAIYYCARERVYTVSFDYWGQGTL | KPGKAPKLLIYDSYILETG |
| | | | | MNWVRQAPGKG | HYLNWYQQKP | VTVSSASTKGPSVFPLAPSSKSTSGGTAALGCLVKDYF | VTSRFSGSGSGTDFTFTIN |
| | | | | LEWVSSISSSS | GKAPKLLIYD | PEPVTVSWNSGALTSGVHTFPAVLQSSGLYSLSSVVTV | SLQPEDIAIYYCQQYDNLP |
| | | | | IYIHSADSVKG | SYILETGVTS | PSSSLGTQTYICNVNHKPSNTKVDKKVEPKSCDKTHTC | YTFGQGTKVEIKRTVAAPS |
| | | | | RFTISRDNAKN | RFSGSGSGTD | PPCPAPELLGGPSVFLFPPKPKDTLMISRTPEVTCVVV | VFIFPPSDEQLKSGTASVV |
| | | | | SLYLQMNSLRA | FTFTINSLQP | DVSHEDPEVKFNWYVDGVEVHNAKTKPREEQYNSTYRV | CLLNNFYPREAKVQWKVDN |
| | | | | EDSAIYYCARE | EDIAIYYCQQ | VSVLTVLHQDWLNGKEYKCKVSNKALPAPIEKTISKAK | ALQSGNSQESVTEQDSKDS |
| | | | | RVYTVSFDYWG | YDNLPYTFGQ | GQPREPQVYTLPPSREEMTKNQVSLTCLVKGFYPSDIA | TYSLSSTLTLSKADYEKHK |
| | | | | QGTLVTVSS | GTKVEIK | VEWESNGQPENNYKTTPPVLDSDGSFFLYSKLTVDKSR | VYACEVTHQGLSSPVTKSF |
| | | | | | | WQQGNVFSCSVMHEALHNHYTQKSLSLSPGK | NRGEC |
| | | | | 2173 | 2174 | 2175 | 2176 |
| 65 | VG9B201 | IgG1 | Lambda | QVTLKESGPTL | QLVLTQPPSV | QVTLKESGPTLVKPTQTLTLTCTFSGFSLTTHGVGVGW | QLVLTQPPSVSAAPGQKVT |
| | | | | VKPTQTLTLTC | SAAPGQKVTI | IRQPPRKALEWLALIYWNADKHYSPSLKSRLTITKDTS | ISCSGSSSNIGNNDVSWYQ |
| | | | | TFSGFSLTTHG | SCSGSSSNIG | KNQVVLTMTHMDPVDTATYYCAHEGDWGHYFDFWGQGT | QLPGTAPKLLIYDNNRRPS |
| | | | | VGVGWIRQPPR | NNDVSWYQQL | LVTVSSASTKGPSVFPLAPSSKSTSGGTAALGCLVKDY | GIPDRFSGSKSGTSATLGI |
| | | | | KALEWLALIYW | PGTAPKLLIY | FPEPVTVSWNSGALTSGVHTFPAVLQSSGLYSLSSVVT | TGLQTGDEADYYCETWDSS |
| | | | | NADKHYSPSLK | DNNRRPSGIP | VPSSSLGTQTYICNVNHKPSNTKVDKKVEPKSCDKTHT | LSAIWVFGGGTKVTVLGQP |
| | | | | SRLTITKDTSK | DRFSGSKSGT | CPPCPAPELLGGPSVFLFPPKPKDTLMISRTPEVTCVV | KAAPSVTLFPPSSEELQAN |
| | | | | NQVVLTMTHMD | SATLGITGLQ | VDVSHEDPEVKFNWYVDGVEVHNAKTKPREEQYNSTYR | KATLVCLISDFYPGAVTVA |
| | | | | PVDTATYYCAH | TGDEADYYCE | VVSVLTVLHQDWLNGKEYKCKVSNKALPAPIEKTISKA | WKADSSPVKAGVETTTPSK |
| | | | | EGDWGHYFDFW | TWDSSLSAIW | KGQPREPQVYTLPPSREEMTKNQVSLTCLVKGFYPSDI | QSNNKYAASSYLSLTPEQW |
| | | | | GQGTLVTVSS | VFGGGTKVTV | AVEWESNGQPENNYKTTPPVLDSDGSFFLYSKLTVDKS | KSHRSYSCQVTHEGSTVEK |
| | | | | | L | RWQQGNVFSCSVMHEALHNHYTQKSLSLSPGK | TVAPTECS |
| | | | | 2207 | 2208 | 2209 | 2210 |
| 66 | VG9B161 | IgG1 | Lambda | QVQLQESGPGL | QPVLTQSSSA | QVQLQESGPGLVKPSETLSLTCTVSGGSISSYFWSWIR | QPVLTQSSSASGTPGQRVT |
| | | | | VKPSETLSLTC | SGTPGQRVTI | QPPGKGLEWIGYIFYSGSTNYNPSLKSRVTILVDTSKN | ISCSGSSSNIGSNTVNWYQ |
| | | | | TVSGGSISSYF | SCSGSSSNIG | HFSLKLSSVTAADTAVYYCARVGRWELRGAFDIWGQGT | QFPGTAPKLLIYSNNQRPS |
| | | | | WSWIRQPPGKG | SNTVNWYQQF | MVTVSSASTKGPSVFPLAPSSKSTSGGTAALGCLVKDY | GVPDRFSGSKSGTSASLAI |
| | | | | LEWIGYIFYSG | PGTAPKLLIY | FPEPVTVSWNSGALTSGVHTFPAVLQSSGLYSLSSVVT | SGLQSEDEAEYYCAAWDDS |
| | | | | STNYNPSLKSR | SNNQRPSGVP | VPSSSLGTQTYICNVNHKPSNTKVDKKVEPKSCDKTHT | LNSPGVFGGGTKLTVLGQP |
| | | | | VTILVDTSKNH | DRFSGSKSGT | CPPCPAPELLGGPSVFLFPPKPKDTLMISRTPEVTCVV | KAAPSVTLFPPSSEELQAN |
| | | | | FSLKLSSVTAA | SASLAISGLQ | VDVSHEDPEVKFNWYVDGVEVHNAKTKPREEQYNSTYR | KATLVCLISDFYPGAVTVA |
| | | | | DTAVYYCARVG | SEDEAEYYCA | VVSVLTVLHQDWLNGKEYKCKVSNKALPAPIEKTISKA | WKADSSPVKAGVETTTPSK |
| | | | | RWELRGAFDIW | AWDDSLNSPG | KGQPREPQVYTLPPSREEMTKNQVSLTCLVKGFYPSDI | QSNNKYAASSYLSLTPEQW |
| | | | | GQGTMVTVSS | VFGGGTKLTV | AVEWESNGQPENNYKTTPPVLDSDGSFFLYSKLTVDKS | KSHRSYSCQVTHEGSTVEK |
| | | | | | L | RWQQGNVFSCSVMHEALHNHYTQKSLSLSPGK | TVAPTECS |
| | | | | 2241 | 2242 | 2243 | 2244 |
| 67 | VG9B383 | IgG1 | Lambda | QVQLQESGPGL | SSELTQPASV | QVQLQESGPGLVKPSETLSLTCTVSSGSISSYYWNWIR | SSELTQPASVSGSPGQSIT |
| | | | | VKPSETLSLTC | SGSPGQSITI | QPAGKGLEWVGRIYTIGNTNYNPSLKSRVTMSIDTSKN | ISCTGTSNDVGSYNLVSWY |
| | | | | TVSSGSISSYY | SCTGTSNDVG | QFSLNLSSVTAADTAVYFCAREGYYDSSGSFFPGAFGI | QQHPGKAPKLMIYAGSKRP |
| | | | | WNWIRQPAGKG | SYNLVSWYQQ | WGQGTMVTVSSASTKGPSVFPLAPSSKSTSGGTAALGC | SGISNRFSGSKSGNTASLT |
| | | | | LEWVGRIYTIG | HPGKAPKLMI | LVKDYFPEPVTVSWNSGALTSGVHTFPAVLQSSGLYSL | ISGLQAEDEADYYCCSFAG |
| | | | | NTNYNPSLKSR | YAGSKRPSGI | SSVVTVPSSSLGTQTYICNVNHKPSNTKVDKKVEPKSC | ATNVVFGGGTKLTVLGQPK |
| | | | | VTMSIDTSKNQ | SNRFSGSKSG | DKTHTCPPCPAPELLGGPSVFLFPPKPKDTLMISRTPE | AAPSVTLFPPSSEELQANK |
| | | | | FSLNLSSVTAA | NTASLTISGL | VTCVVVDVSHEDPEVKFNWYVDGVEVHNAKTKPREEQY | ATLVCLISDFYPGAVTVAW |
| | | | | DTAVYFCAREG | QAEDEADYYC | NSTYRVVSVLTVLHQDWLNGKEYKCKVSNKALPAPIEK | KADSSPVKAGVETTTPSKQ |
| | | | | YYDSSGSFFPG | CSFAGATNVV | TISKAKGQPREPQVYTLPPSREEMTKNQVSLTCLVKGF | SNNKYAASSYLSLTPEQWK |
| | | | | AFGIWGQGTMV | FGGGTKLTVL | YPSDIAVEWESNGQPENNYKTTPPVLDSDGSFFLYSKL | SHRSYSCQVTHEGSTVEKT |
| | | | | TVSS | | TVDKSRWQQGNVFSCSVMHEALHNHYTQKSLSLSPGK | VAPTECS |
| | | | | 2275 | 2276 | 2277 | 2278 |
| 68 | VG9B382 | IgG1 | Lambda | QVQLQESGPGL | QSVLTQPPSV | QVQLQESGPGLVKPSETLSLTCTVSGDSISGYYWNYIR | QSVLTQPPSVSGAPGQRVT |
| | | | | VKPSETLSLTC | SGAPGQRVTI | QPAGKGLDWIGRIFTTGNTNYNPSLKSRVTMSVDTSKN | ISCTGSSSNIGADYDIKWY |
| | | | | TVSGDSISGYY | SCTGSSSNIG | QFSLKLTSVTAADTAVYYCARERWDSSSSALYFDYWGQ | QQLPGTAPKLLIYGNSNRP |
| | | | | WNYIRQPAGKG | ADYDIKWYQQ | GTLVTVSSASTKGPSVFPLAPSSKSTSGGTAALGCLVK | SGVPDRFSGSKSGTSVSLA |
| | | | | LDWIGRIFTTG | LPGTAPKLLI | DYFPEPVTVSWNSGALTSGVHTFPAVLQSSGLYSLSSV | ITGLQAEDEADYYCQSYDS |
| | | | | NTNYNPSLKSR | YGNSNRPSGV | VTVPSSSLGTQTYICNVNHKPSNTKVDKKVEPKSCDKT | SMSGYVVFGGGTKLTVLGQ |
| | | | | VTMSVDTSKNQ | PDRFSGSKSG | HTCPPCPAPELLGGPSVFLFPPKPKDTLMISRTPEVTC | PKAAPSVTLFPPSSEELQA |
| | | | | FSLKLTSVTAA | TSVSLAITGL | VVVDVSHEDPEVKFNWYVDGVEVHNAKTKPREEQYNST | NKATLVCLISDFYPGAVTV |
| | | | | DTAVYYCARER | QAEDEADYYC | YRVVSVLTVLHQDWLNGKEYKCKVSNKALPAPIEKTIS | AWKADSSPVKAGVETTTPS |
| | | | | WDSSSSALYFD | QSYDSSMSGY | KAKGQPREPQVYTLPPSREEMTKNQVSLTCLVKGFYPS | KQSNNKYAASSYLSLTPEQ |
| | | | | YWGQGTLVTVS | VVFGGGTKLT | DIAVEWESNGQPENNYKTTPPVLDSDGSFFLYSKLTVD | WKSHRSYSCQVTHEGSTVE |
| | | | | S | VL | KSRWQQGNVFSCSVMHEALHNHYTQKSLSLSPGK | KTVAPTECS |
| | | | | 2309 | 2310 | 2311 | 2312 |
| 69 | VG9B156 | IgG1 | Lambda | QVQLQESGPGL | QLVLTQPPSA | QVQLQESGPGLMKPSETLSLTCTVSNGSISGYFWNWIR | QLVLTQPPSASGTPGQRVT |
| | | | | MKPSETLSLTC | SGTPGQRVTI | QPPGKGLEWIGYIFYSGSTNYNPSLKSRVTISVDTSKN | ISCSGSTSNIGSDTVNWYQ |
| | | | | TVSNGSISGYF | SCSGSTSNIG | LVSLKLNSVTAADTAVYFCAKLGKWELRTAFDIWGQGT | QLPGTAPKLLIYSNNQRPS |
| | | | | WNWIRQPPGKG | SDTVNWYQQL | MVTVSSASTKGPSVFPLAPSSKSTSGGTAALGCLVKDY | GVPDRFSGSKSGTSASLAI |
| | | | | LEWIGYIFYSG | PGTAPKLLIY | FPEPVTVSWNSGALTSGVHTFPAVLQSSGLYSLSSVVT | SGLQSEDETDYYCAAWDDS |
| | | | | STNYNPSLKSR | SNNQRPSGVP | VPSSSLGTQTYICNVNHKPSNTKVDKKVEPKSCDKTHT | LNGPVFGGGTKVTVLGQPK |
| | | | | VTISVDTSKNL | DRFSGSKSGT | CPPCPAPELLGGPSVFLFPPKPKDTLMISRTPEVTCVV | AAPSVTLFPPSSEELQANK |
| | | | | VSLKLNSVTAA | SASLAISGLQ | VDVSHEDPEVKFNWYVDGVEVHNAKTKPREEQYNSTYR | ATLVCLISDFYPGAVTVAW |
| | | | | DTAVYFCAKLG | SEDETDYYCA | VVSVLTVLHQDWLNGKEYKCKVSNKALPAPIEKTISKA | KADSSPVKAGVETTTPSKQ |
| | | | | KWELRTAFDIW | AWDDSLNGPV | KGQPREPQVYTLPPSREEMTKNQVSLTCLVKGFYPSDI | SNNKYAASSYLSLTPEQWK |
| | | | | GQGTMVTVSS | FGGGTKVTVL | AVEWESNGQPENNYKTTPPVLDSDGSFFLYSKLTVDKS | SHRSYSCQVTHEGSTVEKT |
| | | | | | | RWQQGNVFSCSVMHEALHNHYTQKSLSLSPGK | VAPTECS |
| | | | | 2343 | 2344 | 2345 | 2346 |
| 70 | VG9B205 | IgG1 | Lambda | QVQLVQSGPGL | SSELTQPPSV | QVQLVQSGPGLMKPSETLSLTCTVSNGSISGYFWNWIR | SSELTQPPSVSVSPGQTAS |
| | | | | MKPSETLSLTC | SVSPGQTASI | QPPGKGLEWIGYIFYSGSTNYNPSLKSRVTISVDTSKN | ITCSGDKLGDKYACWYQQK |
| | | | | TVSNGSISGYF | TCSGDKLGDK | LVSLKLNSVTAADTAVYFCAKLGKWELRTAFDIWGQGT | PGQSPVLVIYQDSKRPSGI |
| | | | | WNWIRQPPGKG | YACWYQQKPG | MVTVSSASTKGPSVFPLAPSSKSTSGGTAALGCLVKDY | PERFSGSNSGNTATLTISG |
| | | | | LEWIGYIFYSG | QSPVLVIYQD | FPEPVTVSWNSGALTSGVHTFPAVLQSSGLYSLSSVVT | TQAMDEADYYCQAWDSSTV |
| | | | | STNYNPSLKSR | SKRPSGIPER | VPSSSLGTQTYICNVNHKPSNTKVDKKVEPKSCDKTHT | VFGGGTKLTVLGQPKAAPS |
| | | | | VTISVDTSKNL | FSGSNSGNTA | CPPCPAPELLGGPSVFLFPPKPKDTLMISRTPEVTCVV | VTLFPPSSEELQANKATLV |
| | | | | VSLKLNSVTAA | TLTISGTQAM | VDVSHEDPEVKFNWYVDGVEVHNAKTKPREEQYNSTYR | CLISDFYPGAVTVAWKADS |
| | | | | DTAVYFCAKLG | DEADYYCQAW | VVSVLTVLHQDWLNGKEYKCKVSNKALPAPIEKTISKA | SPVKAGVETTTPSKQSNNK |
| | | | | KWELRTAFDIW | DSSTVVFGGG | KGQPREPQVYTLPPSREEMTKNQVSLTCLVKGFYPSDI | YAASSYLSLTPEQWKSHRS |
| | | | | GQGTMVTVSS | TKLTVL | AVEWESNGQPENNYKTTPPVLDSDGSFFLYSKLTVDKS | YSCQVTHEGSTVEKTVAPT |
| | | | | | | RWQQGNVFSCSVMHEALHNHYTQKSLSLSPGK | ECS |
| | | | | 2377 | 2378 | 2379 | 2380 |
| 71 | VG9B86 | IgG1 | Kappa | QVQLQESGPGL | EIVLTQSPAT | QVQLQESGPGLVKPSETLSLTCTVSGDSISSSYWSWIR | EIVLTQSPATLSLSPGERA |
| | | | | VKPSETLSLTC | LSLSPGERAT | QPAGKGLECIGRFYSSGSTSYNPSLKSRVTMAIDTSKN | TLSCRASQSVSSYLAWYQQ |
| | | | | TVSGDSISSSY | LSCRASQSVS | QFSLKLTSVTAADTAVYYCARYSGSYWYFDLWGRGTTV | RPGQAPRLLIYDTSNRATG |
| | | | | WSWIRQPAGKG | SYLAWYQQRP | TVSSASTKGPSVFPLAPSSKSTSGGTAALGCLVKDYFP | IPARFSGSGSGTDFTLTIS |
| | | | | LECIGRFYSSG | GQAPRLLIYD | EPVTVSWNSGALTSGVHTFPAVLQSSGLYSLSSVVTVP | SLAPEDFAVYYCQQRSDWL |
| | | | | STSYNPSLKSR | TSNRATGIPA | SSSLGTQTYICNVNHKPSNTKVDKKVEPKSCDKTHTCP | LTFGGGTKVEIKRTVAAPS |
| | | | | VTMAIDTSKNQ | RFSGSGSGTD | PCPAPELLGGPSVFLFPPKPKDTLMISRTPEVTCVVVD | VFIFPPSDEQLKSGTASVV |
| | | | | FSLKLTSVTAA | FTLTISSLAP | VSHEDPEVKFNWYVDGVEVHNAKTKPREEQYNSTYRVV | CLLNNFYPREAKVQWKVDN |
| | | | | DTAVYYCARYS | EDFAVYYCQQ | SVLTVLHQDWLNGKEYKCKVSNKALPAPIEKTISKAKG | ALQSGNSQESVTEQDSKDS |
| | | | | GSYWYFDLWGR | RSDWLLTFGG | QPREPQVYTLPPSREEMTKNQVSLTCLVKGFYPSDIAV | TYSLSSTLTLSKADYEKHK |
| | | | | GTTVTVSS | GTKVEIK | EWESNGQPENNYKTTPPVLDSDGSFFLYSKLTVDKSRW | VYACEVTHQGLSSPVTKSF |
| | | | | | | QQGNVFSCSVMHEALHNHYTQKSLSLSPGK | NRGEC |
| | | | | 2411 | 2412 | 2413 | 2414 |
| 72 | VG9B154 | IgG1 | Lambda | QVQLQESGPGL | QSALTQPPSA | QVQLQESGPGLVKPSETLSLTCTVSGGSISYYFWNWIR | QSALTQPPSASGTPGQRVT |
| | | | | VKPSETLSLTC | SGTPGQRVTI | QPPGKGLEWIGYIYYSGSTNYNPSLKSRVTISLDTSKN | ISCSGSSSNIGSNTVNWYQ |
| | | | | TVSGGSISYYF | SCSGSSSNIG | QFSLKLSSVTAADTAVYYCAREGKWELRTTFDIWGQGT | QLPGTAPKLLIYSNNQRPS |
| | | | | WNWIRQPPGKG | SNTVNWYQQL | MVTVSSASTKGPSVFPLAPSSKSTSGGTAALGCLVKDY | GVPDRFSGSKSGTSASLAI |
| | | | | LEWIGYIYYSG | PGTAPKLLIY | FPEPVTVSWNSGALTSGVHTFPAVLQSSGLYSLSSVVT | SGLQSEDEADYYCAAWDDS |
| | | | | STNYNPSLKSR | SNNQRPSGVP | VPSSSLGTQTYICNVNHKPSNTKVDKKVEPKSCDKTHT | LNGPVFGGGTKLTVLGQPK |
| | | | | VTISLDTSKNQ | DRFSGSKSGT | CPPCPAPELLGGPSVFLFPPKPKDTLMISRTPEVTCVV | AAPSVTLFPPSSEELQANK |
| | | | | FSLKLSSVTAA | SASLAISGLQ | VDVSHEDPEVKFNWYVDGVEVHNAKTKPREEQYNSTYR | ATLVCLISDFYPGAVTVAW |
| | | | | DTAVYYCAREG | SEDEADYYCA | VVSVLTVLHQDWLNGKEYKCKVSNKALPAPIEKTISKA | KADSSPVKAGVETTTPSKQ |
| | | | | KWELRTTFDIW | AWDDSLNGPV | KGQPREPQVYTLPPSREEMTKNQVSLTCLVKGFYPSDI | SNNKYAASSYLSLTPEQWK |
| | | | | GQGTMVTVSS | FGGGTKLTVL | AVEWESNGQPENNYKTTPPVLDSDGSFFLYSKLTVDKS | SHRSYSCQVTHEGSTVEKT |
| | | | | | | RWQQGNVFSCSVMHEALHNHYTQKSLSLSPGK | VAPTECS |
| | | | | 2445 | 2446 | 2447 | 2448 |
| 73 | VG9B159 | IgG1 | Lambda | EVQLVQSGPGL | SYELMQPPSV | EVQLVQSGPGLVKPSETLSLTCTVSGGSISSYYWSWIR | SYELMQPPSVSVAPGQTAR |
| | | | | VKPSETLSLTC | SVAPGQTARI | QPAGKGLEWIGRFYTGGRNNYNPSFKSRVTMSVDTSQN | ITCGGNNIGSKSVHWYQQK |
| | | | | TVSGGSISSYY | TCGGNNIGSK | QFSLKLSSVTAADTAVYYCARDMEYYYDRSGYSYWYFD | PGQAPVLVVYDDSDRPSGI |
| | | | | WSWIRQPAGKG | SVHWYQQKPG | LWGRGTTVTVSSASTKGPSVFPLAPSSKSTSGGTAALG | PERFSGSNSGNTATLTISR |
| | | | | LEWIGRFYTGG | QAPVLVVYDD | CLVKDYFPEPVTVSWNSGALTSGVHTFPAVLQSSGLYS | VEAGDEADYYCQVWDSSSD |
| | | | | RNNYNPSFKSR | SDRPSGIPER | LSSVVTVPSSSLGTQTYICNVNHKPSNTKVDKKVEPKS | HVVFGGGTKLTVLGQPKAA |
| | | | | VTMSVDTSQNQ | FSGSNSGNTA | CDKTHTCPPCPAPELLGGPSVFLFPPKPKDTLMISRTP | PSVTLFPPSSEELQANKAT |
| | | | | FSLKLSSVTAA | TLTISRVEAG | EVTCVVVDVSHEDPEVKFNWYVDGVEVHNAKTKPREEQ | LVCLISDFYPGAVTVAWKA |
| | | | | DTAVYYCARDM | DEADYYCQVW | YNSTYRVVSVLTVLHQDWLNGKEYKCKVSNKALPAPIE | DSSPVKAGVETTTPSKQSN |
| | | | | EYYYDRSGYSY | DSSSDHVVFG | KTISKAKGQPREPQVYTLPPSREEMTKNQVSLTCLVKG | NKYAASSYLSLTPEQWKSH |
| | | | | WYFDLWGRGTT | GGTKLTVL | FYPSDIAVEWESNGQPENNYKTTPPVLDSDGSFFLYSK | RSYSCQVTHEGSTVEKTVA |
| | | | | VTVSS | | LTVDKSRWQQGNVFSCSVMHEALHNHYTQKSLSLSPGK | PTECS |
| | | | | 2479 | 2480 | 2481 | 2482 |
| 74 | VG9B465 | IgG1 | Lambda | QVQLQQSGPGL | SSELTQPASV | QVQLQQSGPGLVKPSETLSLTCTVSGGSITSYYWNWIR | SSELTQPASVSGSPGQSIT |
| | | | | VKPSETLSLTC | SGSPGQSITI | QPAGKGLEWIGRIYTIGNTNYNPSLKSRVTMSIDTSKH | ISCTGTSNDVGSYNLVSWY |
| | | | | TVSGGSITSYY | SCTGTSNDVG | QFSLKLSSVTGADTAVYYCAREGYYESDGSFFPGAFNI | QQHPGKAPKLMIYAGSKRP |
| | | | | WNWIRQPAGKG | SYNLVSWYQQ | WGQGTMVTVSSASTKGPSVFPLAPSSKSTSGGTAALGC | SGISNRFSGSKSGNTASLT |
| | | | | LEWIGRIYTIG | HPGKAPKLMI | LVKDYFPEPVTVSWNSGALTSGVHTFPAVLQSSGLYSL | ISGLQAEDEADYYCCSYAG |
| | | | | NTNYNPSLKSR | YAGSKRPSGI | SSVVTVPSSSLGTQTYICNVNHKPSNTKVDKKVEPKSC | TTNVVFGGGTKVTVLGQPK |
| | | | | VTMSIDTSKHQ | SNRFSGSKSG | DKTHTCPPCPAPELLGGPSVFLFPPKPKDTLMISRTPE | AAPSVTLFPPSSEELQANK |
| | | | | FSLKLSSVTGA | NTASLTISGL | VTCVVVDVSHEDPEVKFNWYVDGVEVHNAKTKPREEQY | ATLVCLISDFYPGAVTVAW |
| | | | | DTAVYYCAREG | QAEDEADYYC | NSTYRVVSVLTVLHQDWLNGKEYKCKVSNKALPAPIEK | KADSSPVKAGVETTTPSKQ |
| | | | | YYESDGSFFPG | CSYAGTTNVV | TISKAKGQPREPQVYTLPPSREEMTKNQVSLTCLVKGF | SNNKYAASSYLSLTPEQWK |
| | | | | AFNIWGQGTMV | FGGGTKVTVL | YPSDIAVEWESNGQPENNYKTTPPVLDSDGSFFLYSKL | SHRSYSCQVTHEGSTVEKT |
| | | | | TVSS | | TVDKSRWQQGNVFSCSVMHEALHNHYTQKSLSLSPGK | VAPTECS |
| | | | | 2513 | 2514 | 2515 | 2516 |
| 75 | VG9B194 | IgG1 | Lambda | EVQLLESGAEV | QSVLTQPPSA | EVQLLESGAEVKKPGASVKVSCKASGYTFTGDYMHWVR | QSVLTQPPSASGSPGQSVT |
| | | | | KKPGASVKVSC | SGSPGQSVTI | QAPGQGLEWMGWINPNSGYTNNAEKFQGRVTMTRDTSI | ISCTGTSSDVGGYNYVSWY |
| | | | | KASGYTFTGDY | SCTGTSSDVG | STAYMELSRLSSDDTAVYYCTRELDALDVWGQGTMVTV | QQHPGKAPKLMIYEVSKRP |
| | | | | MHWVRQAPGQG | GYNYVSWYQQ | SSASTKGPSVFPLAPSSKSTSGGTAALGCLVKDYFPEP | SGVPDRFSGSKSGNTASLT |
| | | | | LEWMGWINPNS | HPGKAPKLMI | VTVSWNSGALTSGVHTFPAVLQSSGLYSLSSVVTVPSS | VSGLQAEDEADYYCNSYAG |
| | | | | GYTNNAEKFQG | YEVSKRPSGV | SLGTQTYICNVNHKPSNTKVDKKVEPKSCDKTHTCPPC | SNNFEVFGGGTKVTVLGQP |
| | | | | RVTMTRDTSIS | PDRFSGSKSG | PAPELLGGPSVFLFPPKPKDTLMISRTPEVTCVVVDVS | KAAPSVTLFPPSSEELQAN |
| | | | | TAYMELSRLSS | NTASLTVSGL | HEDPEVKFNWYVDGVEVHNAKTKPREEQYNSTYRVVSV | KATLVCLISDFYPGAVTVA |
| | | | | DDTAVYYCTRE | QAEDEADYYC | LTVLHQDWLNGKEYKCKVSNKALPAPIEKTISKAKGQP | WKADSSPVKAGVETTTPSK |
| | | | | LDALDVWGQGT | NSYAGSNNFE | REPQVYTLPPSREEMTKNQVSLTCLVKGFYPSDIAVEW | QSNNKYAASSYLSLTPEQW |
| | | | | MVTVSS | VFGGGTKVTV | ESNGQPENNYKTTPPVLDSDGSFFLYSKLTVDKSRWQQ | KSHRSYSCQVTHEGSTVEK |
| | | | | | L | GNVFSCSVMHEALHNHYTQKSLSLSPGK | TVAPTECS |
| | | | | 2547 | 2548 | 2549 | 2550 |
| 76 | VG9B182 | IgG1 | Lambda | QVQLVESGPGL | QSALTQPPSA | QVQLVESGPGLVKPSETLSLTCTVSGDSINNHFWSWIR | QSALTQPPSASGTPGQRVT |
| | | | | VKPSETLSLTC | SGTPGQRVTI | QPPGKGLEWIGFVFYNGNTNYNPSLKSRVTISIDTSKS | ISCSGSSSNIGSNTVNWYQ |
| | | | | TVSGDSINNHF | SCSGSSSNIG | QFSLKLTSVTAADTAVYYCARVGRWVLRTAFDIWGQGT | QLPGTAPKLLIYSNNQRPS |
| | | | | WSWIRQPPGKG | SNTVNWYQQL | MVTVSSASTKGPSVFPLAPSSKSTSGGTAALGCLVKDY | GVPARFSGSKSGTSASLAI |
| | | | | LEWIGFVFYNG | PGTAPKLLIY | FPEPVTVSWNSGALTSGVHTFPAVLQSSGLYSLSSVVT | SGLQSEDEADYYCAAWDDS |
| | | | | NTNYNPSLKSR | SNNQRPSGVP | VPSSSLGTQTYICNVNHKPSNTKVDKKVEPKSCDKTHT | LNGPGVFGGGTKLTVLGQP |
| | | | | VTISIDTSKSQ | ARFSGSKSGT | CPPCPAPELLGGPSVFLFPPKPKDTLMISRTPEVTCVV | KAAPSVTLFPPSSEELQAN |
| | | | | FSLKLTSVTAA | SASLAISGLQ | VDVSHEDPEVKFNWYVDGVEVHNAKTKPREEQYNSTYR | KATLVCLISDFYPGAVTVA |
| | | | | DTAVYYCARVG | SEDEADYYCA | VVSVLTVLHQDWLNGKEYKCKVSNKALPAPIEKTISKA | WKADSSPVKAGVETTTPSK |
| | | | | RWVLRTAFDIW | AWDDSLNGPG | KGQPREPQVYTLPPSREEMTKNQVSLTCLVKGFYPSDI | QSNNKYAASSYLSLTPEQW |
| | | | | GQGTMVTVSS | VFGGGTKLTV | AVEWESNGQPENNYKTTPPVLDSDGSFFLYSKLTVDKS | KSHRSYSCQVTHEGSTVEK |
| | | | | | L | RWQQGNVFSCSVMHEALHNHYTQKSLSLSPGK | TVAPTECS |
| | | | | 2581 | 2582 | 2583 | 2584 |
| 77 | VG9B173 | IgG1 | Lambda | QVQLQESGPGL | QSVLTQPPSA | QVQLQESGPGLVKPSETLSLTCTVSGGSINNYFWSWIR | QSVLTQPPSASGTPGQRVT |
| | | | | VKPSETLSLTC | SGTPGQRVTI | QPPGKGLEWIGYIYYSGSTNYNPSLKSRVTISVVTSMN | ISCSGSSSNIESNTVNWYQ |
| | | | | TVSGGSINNYF | SCSGSSSNIE | QFSLKLSSVTAADTAVYYCAREGKWELRSAFDIWGQGT | QLPRTAPKLLIYSNNQRPS |
| | | | | WSWIRQPPGKG | SNTVNWYQQL | MVTVSSASTKGPSVFPLAPSSKSTSGGTAALGCLVKDY | GVPDRFSGSKSGTSASLAI |
| | | | | LEWIGYIYYSG | PRTAPKLLIY | FPEPVTVSWNSGALTSGVHTFPAVLQSSGLYSLSSVVT | SGLQSEDEADYYCTAWDDS |
| | | | | STNYNPSLKSR | SNNQRPSGVP | VPSSSLGTQTYICNVNHKPSNTKVDKKVEPKSCDKTHT | LNGPVFGGGTKVTVLGQPK |
| | | | | VTISVVTSMNQ | DRFSGSKSGT | CPPCPAPELLGGPSVFLFPPKPKDTLMISRTPEVTCVV | AAPSVTLFPPSSEELQANK |
| | | | | FSLKLSSVTAA | SASLAISGLQ | VDVSHEDPEVKFNWYVDGVEVHNAKTKPREEQYNSTYR | ATLVCLISDFYPGAVTVAW |
| | | | | DTAVYYCAREG | SEDEADYYCT | VVSVLTVLHQDWLNGKEYKCKVSNKALPAPIEKTISKA | KADSSPVKAGVETTTPSKQ |
| | | | | KWELRSAFDIW | AWDDSLNGPV | KGQPREPQVYTLPPSREEMTKNQVSLTCLVKGFYPSDI | SNNKYAASSYLSLTPEQWK |
| | | | | GQGTMVTVSS | FGGGTKVTVL | AVEWESNGQPENNYKTTPPVLDSDGSFFLYSKLTVDKS | SHRSYSCQVTHEGSTVEKT |
| | | | | | | RWQQGNVFSCSVMHEALHNHYTQKSLSLSPGK | VAPTECS |
| | | | | 2615 | 2616 | 2617 | 2618 |
| 78 | VG9B87 | IgG1 | Kappa | QVQLVESGAEV | EIVLTQSPSF | QVQLVESGAEVKKPGASVKVSCKASGYTFTSEYIHWVR | EIVLTQSPSFLSASAGDRV |
| | | | | KKPGASVKVSC | LSASAGDRVT | QAPGQGLEWMGIINPSGGSTSYAQRFQGRVTMTRDTST | TITCRASQGISSYLAWYQQ |
| | | | | KASGYTFTSEY | ITCRASQGIS | STVYMEVNSLRSEDTAVYYCARERGYSYGSFDYWGQGT | KPEKAPKLLIYAASTLQSG |
| | | | | IHWVRQAPGQG | SYLAWYQQKP | LVTVSSASTKGPSVFPLAPSSKSTSGGTAALGCLVKDY | VPSRFSGSGSGTEFTLTIS |
| | | | | LEWMGIINPSG | EKAPKLLIYA | FPEPVTVSWNSGALTSGVHTFPAVLQSSGLYSLSSVVT | SLQPEDFATYYCQQFNSYS |
| | | | | GSTSYAQRFQG | ASTLQSGVPS | VPSSSLGTQTYICNVNHKPSNTKVDKKVEPKSCDKTHT | LTFGGGTKLEIKRTVAAPS |
| | | | | RVTMTRDTSTS | RFSGSGSGTE | CPPCPAPELLGGPSVFLFPPKPKDTLMISRTPEVTCVV | VFIFPPSDEQLKSGTASVV |
| | | | | TVYMEVNSLRS | FTLTISSLQP | VDVSHEDPEVKFNWYVDGVEVHNAKTKPREEQYNSTYR | CLLNNFYPREAKVQWKVDN |
| | | | | EDTAVYYCARE | EDFATYYCQQ | VVSVLTVLHQDWLNGKEYKCKVSNKALPAPIEKTISKA | ALQSGNSQESVTEQDSKDS |
| | | | | RGYSYGSFDYW | FNSYSLTFGG | KGQPREPQVYTLPPSREEMTKNQVSLTCLVKGFYPSDI | TYSLSSTLTLSKADYEKHK |
| | | | | GQGTLVTVSS | GTKLEIK | AVEWESNGQPENNYKTTPPVLDSDGSFFLYSKLTVDKS | VYACEVTHQGLSSPVTKSF |
| | | | | | | RWQQGNVFSCSVMHEALHNHYTQKSLSLSPGK | NRGEC |
| | | | | 2649 | 2650 | 2651 | 2652 |
| 79 | VG9B208 | IgG1 | Lambda | QVQLQESGPGL | QSVLTQPPSV | QVQLQESGPGLVKPSETLSLTCTVSGGSISGYYWSWIR | QSVLTQPPSVSAAPGQKVT |
| | | | | VKPSETLSLTC | SAAPGQKVTI | QPPGKGLELIGYIFYSGSINYNPSLKNRVTISLDTSKN | ISCSGSTSNIGNNDVSWYQ |
| | | | | TVSGGSISGYY | SCSGSTSNIG | QFSLKLTSVTAADTAVYYCARVGKWELRSSFDIWGQGT | QLPGTAPKLLIYDNNKRPS |
| | | | | WSWIRQPPGKG | NNDVSWYQQL | MVTVSSASTKGPSVFPLAPSSKSTSGGTAALGCLVKDY | GIPDRFSGSKSGTSATLGI |
| | | | | LELIGYIFYSG | PGTAPKLLIY | FPEPVTVSWNSGALTSGVHTFPAVLQSSGLYSLSSVVT | TGLQTGDEADYYCGTWDSS |
| | | | | SINYNPSLKNR | DNNKRPSGIP | VPSSSLGTQTYICNVNHKPSNTKVDKKVEPKSCDKTHT | LSVWVFGGGTKLTVLGQPK |
| | | | | VTISLDTSKNQ | DRFSGSKSGT | CPPCPAPELLGGPSVFLFPPKPKDTLMISRTPEVTCVV | AAPSVTLFPPSSEELQANK |
| | | | | FSLKLTSVTAA | SATLGITGLQ | VDVSHEDPEVKFNWYVDGVEVHNAKTKPREEQYNSTYR | ATLVCLISDFYPGAVTVAW |
| | | | | DTAVYYCARVG | TGDEADYYCG | VVSVLTVLHQDWLNGKEYKCKVSNKALPAPIEKTISKA | KADSSPVKAGVETTTPSKQ |
| | | | | KWELRSSFDIW | TWDSSLSVWV | KGQPREPQVYTLPPSREEMTKNQVSLTCLVKGFYPSDI | SNNKYAASSYLSLTPEQWK |
| | | | | GQGTMVTVSS | FGGGTKLTVL | AVEWESNGQPENNYKTTPPVLDSDGSFFLYSKLTVDKS | SHRSYSCQVTHEGSTVEKT |
| | | | | | | RWQQGNVFSCSVMHEALHNHYTQKSLSLSPGK | VAPTECS |
| | | | | 2683 | 2684 | 2685 | 2686 |
| 80 | VG9B372 | IgG1 | Kappa | QVQLQESGPGL | EIVMTQSPAT | QVQLQESGPGLVKPSQTLSLTCTVSGGSISNGGFYWSW | EIVMTQSPATLSLSPGEGA |
| | | | | VKPSQTLSLTC | LSLSPGEGAT | IRQHPGKGLEWLGYINYSGSTYYNPSLESRGTISLDTS | TLSCRASQSVSSYLAWYQQ |
| | | | | TVSGGSISNGG | LSCRASQSVS | KNQFSLKLRSVTAADTAVYYCARDRNYEWNFDLWGRGT | KPGQAPRLLVYDASNRATG |
| | | | | FYWSWIRQHPG | SYLAWYQQKP | LVTVSSASTKGPSVFPLAPSSKSTSGGTAALGCLVKDY | IPARFSGSGSGTAFTLTIT |
| | | | | KGLEWLGYINY | GQAPRLLVYD | FPEPVTVSWNSGALTSGVHTFPAVLQSSGLYSLSSVVT | SLEPEDFVVYYCQQRSNWP |
| | | | | SGSTYYNPSLE | ASNRATGIPA | VPSSSLGTQTYICNVNHKPSNTKVDKKVEPKSCDKTHT | LTFGGGTKLEIKRTVAAPS |
| | | | | SRGTISLDTSK | RFSGSGSGTA | CPPCPAPELLGGPSVFLFPPKPKDTLMISRTPEVTCVV | VFIFPPSDEQLKSGTASVV |
| | | | | NQFSLKLRSVT | FTLTITSLEP | VDVSHEDPEVKFNWYVDGVEVHNAKTKPREEQYNSTYR | CLLNNFYPREAKVQWKVDN |
| | | | | AADTAVYYCAR | EDFVVYYCQQ | VVSVLTVLHQDWLNGKEYKCKVSNKALPAPIEKTISKA | ALQSGNSQESVTEQDSKDS |
| | | | | DRNYEWNFDLW | RSNWPLTFGG | KGQPREPQVYTLPPSREEMTKNQVSLTCLVKGFYPSDI | TYSLSSTLTLSKADYEKHK |
| | | | | GRGTLVTVSS | GTKLEIK | AVEWESNGQPENNYKTTPPVLDSDGSFFLYSKLTVDKS | VYACEVTHQGLSSPVTKSF |
| | | | | | | RWQQGNVFSCSVMHEALHNHYTQKSLSLSPGK | NRGEC |
| | | | | 2717 | 2718 | 2719 | 2720 |
| 81 | VG9B186 | IgG1 | Lambda | QVTLRESGPVL | QSVLTQPPSA | QVTLRESGPVLVKPTETLTLTCTVSGFSLTNIRMSVSW | QSVLTQPPSASGTPGQRVT |
| | | | | VKPTETLTLTC | SGTPGQRVTI | IRQPPGEALEWLAHIFSNDEKSYNSSLKSRLTISRDTS | ISCSGSTSNIGSNTVNWYQ |
| | | | | TVSGFSLTNIR | SCSGSTSNIG | KSQVVLTLTNVDPVDTATYYCARMRLPYGMDVWGQGTM | QLPGTAPKLLIYSNNQRPS |
| | | | | MSVSWIRQPPG | SNTVNWYQQL | VTVSSASTKGPSVFPLAPSSKSTSGGTAALGCLVKDYF | GVPDRFSGSKSGTSASLAI |
| | | | | EALEWLAHIFS | PGTAPKLLIY | PEPVTVSWNSGALTSGVHTFPAVLQSSGLYSLSSVVTV | SGLQSEDEADFYCAAWDDS |
| | | | | NDEKSYNSSLK | SNNQRPSGVP | PSSSLGTQTYICNVNHKPSNTKVDKKVEPKSCDKTHTC | LNGPVFGGGTKVTVLGQPK |
| | | | | SRLTISRDTSK | DRFSGSKSGT | PPCPAPELLGGPSVFLFPPKPKDTLMISRTPEVTCVVV | AAPSVTLFPPSSEELQANK |
| | | | | SQVVLTLTNVD | SASLAISGLQ | DVSHEDPEVKFNWYVDGVEVHNAKTKPREEQYNSTYRV | ATLVCLISDFYPGAVTVAW |
| | | | | PVDTATYYCAR | SEDEADFYCA | VSVLTVLHQDWLNGKEYKCKVSNKALPAPIEKTISKAK | KADSSPVKAGVETTTPSKQ |
| | | | | MRLPYGMDVWG | AWDDSLNGPV | GQPREPQVYTLPPSREEMTKNQVSLTCLVKGFYPSDIA | SNNKYAASSYLSLTPEQWK |
| | | | | QGTMVTVSS | FGGGTKVTVL | VEWESNGQPENNYKTTPPVLDSDGSFFLYSKLTVDKSR | SHRSYSCQVTHEGSTVEKT |
| | | | | | | WQQGNVFSCSVMHEALHNHYTQKSLSLSPGK | VAPTECS |
| | | | | 2751 | 2752 | 2753 | 2754 |
| 82 | VG9B177 | IgG1 | Lambda | QVQLQESGPGL | QSALTQPASV | QVQLQESGPGLVKPSETLSLTCTVSGGSINNYFWSWIR | QSALTQPASVSGSPGQSIT |
| | | | | VKPSETLSLTC | SGSPGQSITI | QPPGKGLEWIGYIFYSGSTNYNPSLKSRVTISIDTSKN | ISCTGTSSDVADYNYVSWY |
| | | | | TVSGGSINNYF | SCTGTSSDVA | QFSLKVNSVTAADTAVYYCARVGKWELRTAFDIWGQGT | QQHPGKAPKLMIYEVSNRP |
| | | | | WSWIRQPPGKG | DYNYVSWYQQ | MVTVSSASTKGPSVFPLAPSSKSTSGGTAALGCLVKDY | SGVSNRFSGSKSGNTASLT |
| | | | | LEWIGYIFYSG | HPGKAPKLMI | FPEPVTVSWNSGALTSGVHTFPAVLQSSGLYSLSSVVT | ISGLQAEDEADYYCCSYTS |
| | | | | STNYNPSLKSR | YEVSNRPSGV | VPSSSLGTQTYICNVNHKPSNTKVDKKVEPKSCDKTHT | SFTVVFGGGTKVTVLGQPK |
| | | | | VTISIDTSKNQ | SNRFSGSKSG | CPPCPAPELLGGPSVFLFPPKPKDTLMISRTPEVTCVV | AAPSVTLFPPSSEELQANK |
| | | | | FSLKVNSVTAA | NTASLTISGL | VDVSHEDPEVKFNWYVDGVEVHNAKTKPREEQYNSTYR | ATLVCLISDFYPGAVTVAW |
| | | | | DTAVYYCARVG | QAEDEADYYC | VVSVLTVLHQDWLNGKEYKCKVSNKALPAPIEKTISKA | KADSSPVKAGVETTTPSKQ |
| | | | | KWELRTAFDIW | CSYTSSFTVV | KGQPREPQVYTLPPSREEMTKNQVSLTCLVKGFYPSDI | SNNKYAASSYLSLTPEQWK |
| | | | | GQGTMVTVSS | FGGGTKVTVL | AVEWESNGQPENNYKTTPPVLDSDGSFFLYSKLTVDKS | SHRSYSCQVTHEGSTVEKT |
| | | | | | | RWQQGNVFSCSVMHEALHNHYTQKSLSLSPGK | VAPTECS |
| | | | | 2785 | 2786 | 2787 | 2788 |
| 83 | VG9B114 | IgG1 | Kappa | EVQLVESGGDL | DIVMTQSPSF | EVQLVESGGDLVQPGGSLRLSCAASGFTFSRYDMHWVR | DIVMTQSPSFLSASVGDRV |
| | | | | VQPGGSLRLSC | LSASVGDRVT | QATGKGLEWVSAIGSAGDTYYPGSVKGRFTISRENAKN | TITCRASQGIHSYLAWYQQ |
| | | | | AASGFTFSRYD | ITCRASQGIH | SLYLQMNSLRAGDTAVYYCARGKWELRDAFDIWGQGTM | KPGKAPKLLIYVASTLQSG |
| | | | | MHWVRQATGKG | SYLAWYQQKP | VTVSSASTKGPSVFPLAPSSKSTSGGTAALGCLVKDYF | VPSRFSGSRSGTEFTLTIS |
| | | | | LEWVSAIGSAG | GKAPKLLIYV | PEPVTVSWNSGALTSGVHTFPAVLQSSGLYSLSSVVTV | SLQPEDFATYYCQQLNSYP |
| | | | | DTYYPGSVKGR | ASTLQSGVPS | PSSSLGTQTYICNVNHKPSNTKVDKKVEPKSCDKTHTC | YTFGQGTKLEIKRTVAAPS |
| | | | | FTISRENAKNS | RFSGSRSGTE | PPCPAPELLGGPSVFLFPPKPKDTLMISRTPEVTCVVV | VFIFPPSDEQLKSGTASVV |
| | | | | LYLQMNSLRAG | FTLTISSLQP | DVSHEDPEVKFNWYVDGVEVHNAKTKPREEQYNSTYRV | CLLNNFYPREAKVQWKVDN |
| | | | | DTAVYYCARGK | EDFATYYCQQ | VSVLTVLHQDWLNGKEYKCKVSNKALPAPIEKTISKAK | ALQSGNSQESVTEQDSKDS |
| | | | | WELRDAFDIWG | LNSYPYTFGQ | GQPREPQVYTLPPSREEMTKNQVSLTCLVKGFYPSDIA | TYSLSSTLTLSKADYEKHK |
| | | | | QGTMVTVSS | GTKLEIK | VEWESNGQPENNYKTTPPVLDSDGSFFLYSKLTVDKSR | VYACEVTHQGLSSPVTKSF |
| | | | | | | WQQGNVFSCSVMHEALHNHYTQKSLSLSPGK | NRGEC |
| | | | | 2819 | 2820 | 2821 | 2822 |
| 84 | VG9B147 | IgG1 | Lambda | QVQLQESGPGL | SSELTQDPAV | QVQLQESGPGLVNPSETLSLTCTVSGGSISGYFWNWIR | SSELTQDPAVSVALGQTVR |
| | | | | VNPSETLSLTC | SVALGQTVRI | QPPGKGLEWIGYIFYSGSTNYNPSLKSRVTISVDTSKN | ITCQGDSLRSYYATWYQQK |
| | | | | TVSGGSISGYF | TCQGDSLRSY | QFSLKLSSVTAADTAIYYCAREGKWELRSTFDIWGQGT | PGQAPILVIYGENNRPSGI |
| | | | | WNWIRQPPGKG | YATWYQQKPG | MVTVSSASTKGPSVFPLAPSSKSTSGGTAALGCLVKDY | PDRFSGSRSGNTASLTITG |
| | | | | LEWIGYIFYSG | QAPILVIYGE | FPEPVTVSWNSGALTSGVHTFPAVLQSSGLYSLSSVVT | AQAEDEADYYCNSRDTGDH |
| | | | | STNYNPSLKSR | NNRPSGIPDR | VPSSSLGTQTYICNVNHKPSNTKVDKKVEPKSCDKTHT | HLVFGGGTKLTVLGQPKAA |
| | | | | VTISVDTSKNQ | FSGSRSGNTA | CPPCPAPELLGGPSVFLFPPKPKDTLMISRTPEVTCVV | PSVTLFPPSSEELQANKAT |
| | | | | FSLKLSSVTAA | SLTITGAQAE | VDVSHEDPEVKFNWYVDGVEVHNAKTKPREEQYNSTYR | LVCLISDFYPGAVTVAWKA |
| | | | | DTAIYYCAREG | DEADYYCNSR | VVSVLTVLHQDWLNGKEYKCKVSNKALPAPIEKTISKA | DSSPVKAGVETTTPSKQSN |
| | | | | KWELRSTFDIW | DTGDHHLVFG | KGQPREPQVYTLPPSREEMTKNQVSLTCLVKGFYPSDI | NKYAASSYLSLTPEQWKSH |
| | | | | GQGTMVTVSS | GGTKLTVL | AVEWESNGQPENNYKTTPPVLDSDGSFFLYSKLTVDKS | RSYSCQVTHEGSTVEKTVA |
| | | | | | | RWQQGNVFSCSVMHEALHNHYTQKSLSLSPGK | PTECS |
| | | | | 2853 | 2854 | 2855 | 2856 |
| 85 | VG9B65 | IgG1 | Kappa | QVQLVQSGAEV | DIVMTQTPLS | QVQLVQSGAEVKKPGSSVKVSCKASGGTFNIYAINWVR | DIVMTQTPLSSPVTLGQPA |
| | | | | KKPGSSVKVSC | SPVTLGQPAS | QAPGQGLEWMGGIIPFFGTANYAQKFQDRVTITADKST | SISCRSSQSLVHSDGNTYL |
| | | | | KASGGTFNIYA | ISCRSSQSLV | NTAYMELSSLRSEDTAVFYCARGGDSGYDWGFDYWGQG | SWLQQRPGQPPRLLIYQIS |
| | | | | INWVRQAPGQG | HSDGNTYLSW | TLVTVSSASTKGPSVFPLAPSSKSTSGGTAALGCLVKD | NRFSGVPDRFSGSGAGTDF |
| | | | | LEWMGGIIPFF | LQQRPGQPPR | YFPEPVTVSWNSGALTSGVHTFPAVLQSSGLYSLSSVV | TLNISRVEADDVGVYYCMQ |
| | | | | GTANYAQKFQD | LLIYQISNRF | TVPSSSLGTQTYICNVNHKPSNTKVDKKVEPKSCDKTH | ATQFPLTFGGGTKVEIKRT |
| | | | | RVTITADKSTN | SGVPDRFSGS | TCPPCPAPELLGGPSVFLFPPKPKDTLMISRTPEVTCV | VAAPSVFIFPPSDEQLKSG |
| | | | | TAYMELSSLRS | GAGTDFTLNI | VVDVSHEDPEVKFNWYVDGVEVHNAKTKPREEQYNSTY | TASVVCLLNNFYPREAKVQ |
| | | | | EDTAVFYCARG | SRVEADDVGV | RVVSVLTVLHQDWLNGKEYKCKVSNKALPAPIEKTISK | WKVDNALQSGNSQESVTEQ |
| | | | | GDSGYDWGFDY | YYCMQATQFP | AKGQPREPQVYTLPPSREEMTKNQVSLTCLVKGFYPSD | DSKDSTYSLSSTLTLSKAD |
| | | | | WGQGTLVTVSS | LTFGGGTKVE | IAVEWESNGQPENNYKTTPPVLDSDGSFFLYSKLTVDK | YEKHKVYACEVTHQGLSSP |
| | | | | | IK | SRWQQGNVFSCSVMHEALHNHYTQKSLSLSPGK | VTKSFNRGEC |
| | | | | 2887 | 2888 | 2889 | 2890 |
| 86 | VG9B81 | IgG1 | Kappa | QVQLQESGPGL | DIQMTQSPSF | QVQLQESGPGLVKPSETLSLTCSVSGGSINTYYWSWFR | DIQMTQSPSFLSASVRDRV |
| | | | | VKPSETLSLTC | LSASVRDRVN | QPAGKGLELIGRIYTSDNTNYNPSLKSRVTMSVDTSKN | NITCRASQGISSYLAWYQQ |
| | | | | SVSGGSINTYY | ITCRASQGIS | QFSLKLSSVTAADTAVYYCARYNWNYWYFDLWGRGTLV | KPGKAPKLLIYTASTLQSG |
| | | | | WSWFRQPAGKG | SYLAWYQQKP | TVSSASTKGPSVFPLAPSSKSTSGGTAALGCLVKDYFP | VPSRFSGSGSGTEFTLTIS |
| | | | | LELIGRIYTSD | GKAPKLLIYT | EPVTVSWNSGALTSGVHTFPAVLQSSGLYSLSSVVTVP | SLQPEDFATYYCQHLNSYP |
| | | | | NTNYNPSLKSR | ASTLQSGVPS | SSSLGTQTYICNVNHKPSNTKVDKKVEPKSCDKTHTCP | YTFGQGTKVEIKRTVAAPS |
| | | | | VTMSVDTSKNQ | RFSGSGSGTE | PCPAPELLGGPSVFLFPPKPKDTLMISRTPEVTCVVVD | VFIFPPSDEQLKSGTASVV |
| | | | | FSLKLSSVTAA | FTLTISSLQP | VSHEDPEVKFNWYVDGVEVHNAKTKPREEQYNSTYRVV | CLLNNFYPREAKVQWKVDN |
| | | | | DTAVYYCARYN | EDFATYYCQH | SVLTVLHQDWLNGKEYKCKVSNKALPAPIEKTISKAKG | ALQSGNSQESVTEQDSKDS |
| | | | | WNYWYFDLWGR | LNSYPYTFGQ | QPREPQVYTLPPSREEMTKNQVSLTCLVKGFYPSDIAV | TYSLSSTLTLSKADYEKHK |
| | | | | GTLVTVSS | GTKVEIK | EWESNGQPENNYKTTPPVLDSDGSFFLYSKLTVDKSRW | VYACEVTHQGLSSPVTKSF |
| | | | | | | QQGNVFSCSVMHEALHNHYTQKSLSLSPGK | NRGEC |
| | | | | 2921 | 2922 | 2923 | 2924 |
| 87 | VG9B203 | IgG1 | Lambda | QVQLVQSGPGL | QSVLTQPPSV | QVQLVQSGPGLVKPSETLSLTCTVSGDSIGHYYWNWIR | QSVLTQPPSVSAAPGQKVT |
| | | | | VKPSETLSLTC | SAAPGQKVTI | QPAGKGLEWIGRIYTSGSTNYNPSLKSRVTMSVDTSKN | ISCSGSSSNIGNNYVSWYQ |
| | | | | TVSGDSIGHYY | SCSGSSSNIG | QFSLKLSSVTAADTAVYYCARSGGNFYWYFDLWGRGTL | QVPGTAPKLLIYDNNKRPS |
| | | | | WNWIRQPAGKG | NNYVSWYQQV | VTVSSASTKGPSVFPLAPSSKSTSGGTAALGCLVKDYF | GIPDRISGSKSGTSATLGI |
| | | | | LEWIGRIYTSG | PGTAPKLLIY | PEPVTVSWNSGALTSGVHTFPAVLQSSGLYSLSSVVTV | TGLQTGDEADYYCGTWDSS |
| | | | | STNYNPSLKSR | DNNKRPSGIP | PSSSLGTQTYICNVNHKPSNTKVDKKVEPKSCDKTHTC | LSAGVFGGGTKVTVLGQPK |
| | | | | VTMSVDTSKNQ | DRISGSKSGT | PPCPAPELLGGPSVFLFPPKPKDTLMISRTPEVTCVVV | AAPSVTLFPPSSEELQANK |
| | | | | FSLKLSSVTAA | SATLGITGLQ | DVSHEDPEVKFNWYVDGVEVHNAKTKPREEQYNSTYRV | ATLVCLISDFYPGAVTVAW |
| | | | | DTAVYYCARSG | TGDEADYYCG | VSVLTVLHQDWLNGKEYKCKVSNKALPAPIEKTISKAK | KADSSPVKAGVETTTPSKQ |
| | | | | GNFYWYFDLWG | TWDSSLSAGV | GQPREPQVYTLPPSREEMTKNQVSLTCLVKGFYPSDIA | SNNKYAASSYLSLTPEQWK |
| | | | | RGTLVTVSS | FGGGTKVTVL | VEWESNGQPENNYKTTPPVLDSDGSFFLYSKLTVDKSR | SHRSYSCQVTHEGSTVEKT |
| | | | | | | WQQGNVFSCSVMHEALHNHYTQKSLSLSPGK | VAPTECS |
| | | | | 2955 | 2956 | 2957 | 2958 |
| 88 | VG9B380 | IgG1 | Lambda | QVQLQESGPGL | QSALTQPASV | QVQLQESGPGLVKPSETLSLTCTVSGGSISNYYWSWIR | QSALTQPASVSGSPGQSIT |
| | | | | VKPSETLSLTC | SGSPGQSITI | QPAGKGLEWIGRIYPSGITSYDPSLKSRVTMSLDTSKN | ISCTGTSSDVGGYNYVSWY |
| | | | | TVSGGSISNYY | SCTGTSSDVG | QFSLKLTSVTAADTAVYYCAGIMGTKGAFDIWGQGTMV | QQHPGKAPKLLIYEVSNRP |
| | | | | WSWIRQPAGKG | GYNYVSWYQQ | TVSSASTKGPSVFPLAPSSKSTSGGTAALGCLVKDYFP | SGVSNRFSGSKSGSTASLT |
| | | | | LEWIGRIYPSG | HPGKAPKLLI | EPVTVSWNSGALTSGVHTFPAVLQSSGLYSLSSVVTVP | ISGLQAEDEADYYCSSYTS |
| | | | | ITSYDPSLKSR | YEVSNRPSGV | SSSLGTQTYICNVNHKPSNTKVDKKVEPKSCDKTHTCP | TSVVFGGGTKLTVLGQPKA |
| | | | | VTMSLDTSKNQ | SNRFSGSKSG | PCPAPELLGGPSVFLFPPKPKDTLMISRTPEVTCVVVD | APSVTLFPPSSEELQANKA |
| | | | | FSLKLTSVTAA | STASLTISGL | VSHEDPEVKFNWYVDGVEVHNAKTKPREEQYNSTYRVV | TLVCLISDFYPGAVTVAWK |
| | | | | DTAVYYCAGIM | QAEDEADYYC | SVLTVLHQDWLNGKEYKCKVSNKALPAPIEKTISKAKG | ADSSPVKAGVETTTPSKQS |
| | | | | GTKGAFDIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 2989 | 2990 | 2991 | 2992 |
| 89 | VG9B103 | IgG1 | Kappa | EVQLVQSGAEV | ETTLTQSPLS | EVQLVQSGAEVKKPGSSVKVSCKTSGGTFSSYAISWVR | ETTLTQSPLSSPVTLGQPA |
| | | | | KKPGSSVKVSC | SPVTLGQPAS | QAPGQGLEWMGGIIPIFGTATYAQKFQDRVTITADKST | SISCRSSQGLVHSDGNTYL |
| | | | | KTSGGTFSSYA | ISCRSSQGLV | STAYMELSSLRSEDTAVFYCARGVGWGTDYYYGLDVWG | SWLQQRPGQSPRLLIYKIS |
| | | | | ISWVRQAPGQG | HSDGNTYLSW | QGTTVTVSSASTKGPSVFPLAPSSKSTSGGTAALGCLV | NRFSGVPDRFSGSGAGTDF |
| | | | | LEWMGGIIPIF | LQQRPGQSPR | KDYFPEPVTVSWNSGALTSGVHTFPAVLQSSGLYSLSS | TLKISRVEAEDVGVYYCMQ |
| | | | | GTATYAQKFQD | LLIYKISNRF | VVTVPSSSLGTQTYICNVNHKPSNTKVDKKVEPKSCDK | ATHHPLTFGGGTKVEIKRT |
| | | | | RVTITADKSTS | SGVPDRFSGS | THTCPPCPAPELLGGPSVFLFPPKPKDTLMISRTPEVT | VAAPSVFIFPPSDEQLKSG |
| | | | | TAYMELSSLRS | GAGTDFTLKI | CVVVDVSHEDPEVKFNWYVDGVEVHNAKTKPREEQYNS | TASVVCLLNNFYPREAKVQ |
| | | | | EDTAVFYCARG | SRVEAEDVGV | TYRVVSVLTVLHQDWLNGKEYKCKVSNKALPAPIEKTI | WKVDNALQSGNSQESVTEQ |
| | | | | VGWGTDYYYGL | YYCMQATHHP | SKAKGQPREPQVYTLPPSREEMTKNQVSLTCLVKGFYP | DSKDSTYSLSSTLTLSKAD |
| | | | | DVWGQGTTVTV | LTFGGGTKVE | SDIAVEWESNGQPENNYKTTPPVLDSDGSFFLYSKLTV | YEKHKVYACEVTHQGLSSP |
| | | | | SS | IK | DKSRWQQGNVFSCSVMHEALHNHYTQKSLSLSPGK | VTKSFNRGEC |
| | | | | 3023 | 3024 | 3025 | 3026 |
| 90 | VG9B462 | IgG1 | Lambda | QVQLQQSGPGL | QSVLTQPASV | QVQLQQSGPGLVKPSETLSLTCTVSGGSISNYYWSWIR | QSVLTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3057 | 3058 | 3059 | 3060 |
| 91 | VG9B461 | IgG1 | Lambda | QVTLKESGPGL | QPVLTQSSSV | QVTLKESGPGLVKPSETLSLTCTVSGGSISNYYWSWIR | QPVLTQSSSVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKVTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKVTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3091 | 3092 | 3093 | 3094 |
| 92 | VG9B106 | IgG1 | Kappa | EVQLVQSGAEV | DIVMTQSPDS | EVQLVQSGAEVKKPGASVKVSCKASGYTFTGYYLHWVR | DIVMTQSPDSLAVSLGERA |
| | | | | KKPGASVKVSC | LAVSLGERAT | QAPGQGLEWVGWINPSSGDTDYAQTFQGRVTMTRDTSI | TINCKSSQSVLYSSNNKNY |
| | | | | KASGYTFTGYY | INCKSSQSVL | STAYMELSRLRSDDTAVYYCANELGIGVFDYWGQGTLV | LGWYQQKPGQPPKLLIYWA |
| | | | | LHWVRQAPGQG | YSSNNKNYLG | TVSSASTKGPSVFPLAPSSKSTSGGTAALGCLVKDYFP | STRESGVPDRFSGSGSGTD |
| | | | | LEWVGWINPSS | WYQQKPGQPP | EPVTVSWNSGALTSGVHTFPAVLQSSGLYSLSSVVTVP | FTLTISSLQAEDVAVYFCQ |
| | | | | GDTDYAQTFQG | KLLIYWASTR | SSSLGTQTYICNVNHKPSNTKVDKKVEPKSCDKTHTCP | QYYSIPYTFGQGTKVDIKR |
| | | | | RVTMTRDTSIS | ESGVPDRFSG | PCPAPELLGGPSVFLFPPKPKDTLMISRTPEVTCVVVD | TVAAPSVFIFPPSDEQLKS |
| | | | | TAYMELSRLRS | SGSGTDFTLT | VSHEDPEVKFNWYVDGVEVHNAKTKPREEQYNSTYRVV | GTASVVCLLNNFYPREAKV |
| | | | | DDTAVYYCANE | ISSLQAEDVA | SVLTVLHQDWLNGKEYKCKVSNKALPAPIEKTISKAKG | QWKVDNALQSGNSQESVTE |
| | | | | LGIGVFDYWGQ | VYFCQQYYSI | QPREPQVYTLPPSREEMTKNQVSLTCLVKGFYPSDIAV | QDSKDSTYSLSSTLTLSKA |
| | | | | GTLVTVSS | PYTFGQGTKV | EWESNGQPENNYKTTPPVLDSDGSFFLYSKLTVDKSRW | DYEKHKVYACEVTHQGLSS |
| | | | | | DIK | QQGNVFSCSVMHEALHNHYTQKSLSLSPGK | PVTKSFNRGEC |
| | | | | 3125 | 3126 | 3127 | 3128 |
| 93 | VG9B115 | IgG1 | Kappa | QVTLRESGPTL | EIVLTQSPAT | QVTLRESGPTLVKPTQTLTLTCTFSGFSLSTYGVGVGW | EIVLTQSPATLSLSPGERA |
| | | | | VKPTQTLTLTC | LSLSPGERAT | IRQPPGKALEWLALIYWNDDKRYNPSLKSRLTITKDTS | TLSCRASQSVSSYLAWYQQ |
| | | | | TFSGFSLSTYG | LSCRASQSVS | KNQVVLTMTNMDPVGTATYYCSHESDWSYYFDYWGQGT | KPGLAPRLLIYDASNRATG |
| | | | | VGVGWIRQPPG | SYLAWYQQKP | MVTVSSASTKGPSVFPLAPSSKSTSGGTAALGCLVKDY | IPARFSGSGSGTDFTLTIS |
| | | | | KALEWLALIYW | GLAPRLLIYD | FPEPVTVSWNSGALTSGVHTFPAVLQSSGLYSLSSVVT | SLEPVDFAVYYCQQRSSWP |
| | | | | NDDKRYNPSLK | ASNRATGIPA | VPSSSLGTQTYICNVNHKPSNTKVDKKVEPKSCDKTHT | WTFGQGTKVDIKRTVAAPS |
| | | | | SRLTITKDTSK | RFSGSGSGTD | CPPCPAPELLGGPSVFLFPPKPKDTLMISRTPEVTCVV | VFIFPPSDEQLKSGTASVV |
| | | | | NQVVLTMTNMD | FTLTISSLEP | VDVSHEDPEVKFNWYVDGVEVHNAKTKPREEQYNSTYR | CLLNNFYPREAKVQWKVDN |
| | | | | PVGTATYYCSH | VDFAVYYCQQ | VVSVLTVLHQDWLNGKEYKCKVSNKALPAPIEKTISKA | ALQSGNSQESVTEQDSKDS |
| | | | | ESDWSYYFDYW | RSSWPWTFGQ | KGQPREPQVYTLPPSREEMTKNQVSLTCLVKGFYPSDI | TYSLSSTLTLSKADYEKHK |
| | | | | GQGTMVTVSS | GTKVDIK | AVEWESNGQPENNYKTTPPVLDSDGSFFLYSKLTVDKS | VYACEVTHQGLSSPVTKSF |
| | | | | | | RWQQGNVFSCSVMHEALHNHYTQKSLSLSPGK | NRGEC |
| | | | | 3159 | 3160 | 3161 | 3162 |
| 94 | VG9B27 | IgG1 | Kappa | EVQLVQSGAEV | DIVMTQTPLS | EVQLVQSGAEVKKPGSSVKVSCKASGGTFSSYVISWVR | DIVMTQTPLSSPVTLGQPA |
| | | | | KKPGSSVKVSC | SPVTLGQPAS | QAPGQGLEWMGGILPILSTANYAQKFQGRVTITADKST | SISCRSSQSLVHSDGNTYL |
| | | | | KASGGTFSSYV | ISCRSSQSLV | STAYMELSSLRSEDTAVYYCARAHDYYYGMDVWGQGTM | SWLQQRPGQPPRLLVYKIS |
| | | | | ISWVRQAPGQG | HSDGNTYLSW | VTVSSASTKGPSVFPLAPSSKSTSGGTAALGCLVKDYF | NRFSGVPDRFSGSGAGTDF |
| | | | | LEWMGGILPIL | LQQRPGQPPR | PEPVTVSWNSGALTSGVHTFPAVLQSSGLYSLSSVVTV | TLKISRVEAEDVGIYYCMQ |
| | | | | STANYAQKFQG | LLVYKISNRF | PSSSLGTQTYICNVNHKPSNTKVDKKVEPKSCDKTHTC | ATHHPLTFGGGTKLEIKRT |
| | | | | RVTITADKSTS | SGVPDRFSGS | PPCPAPELLGGPSVFLFPPKPKDTLMISRTPEVTCVVV | VAAPSVFIFPPSDEQLKSG |
| | | | | TAYMELSSLRS | GAGTDFTLKI | DVSHEDPEVKFNWYVDGVEVHNAKTKPREEQYNSTYRV | TASVVCLLNNFYPREAKVQ |
| | | | | EDTAVYYCARA | SRVEAEDVGI | VSVLTVLHQDWLNGKEYKCKVSNKALPAPIEKTISKAK | WKVDNALQSGNSQESVTEQ |
| | | | | HDYYYGMDVWG | YYCMQATHHP | GQPREPQVYTLPPSREEMTKNQVSLTCLVKGFYPSDIA | DSKDSTYSLSSTLTLSKAD |
| | | | | QGTMVTVSS | LTFGGGTKLE | VEWESNGQPENNYKTTPPVLDSDGSFFLYSKLTVDKSR | YEKHKVYACEVTHQGLSSP |
| | | | | | IK | WQQGNVFSCSVMHEALHNHYTQKSLSLSPGK | VTKSFNRGEC |
| | | | | 3193 | 3194 | 3195 | 3196 |
| 95 | VG9B458 | IgG1 | Lambda | QVQLVQSGPGL | QSVLTQPASV | QVQLVQSGPGLVKPSETLSLTCTVSGGSISNYYWSWIR | QSVLTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3227 | 3228 | 3229 | 3230 |
| 96 | VG9B131 | IgG1 | Kappa | QVQLVQSGGTL | EIVMTQSPAT | QVQLVQSGGTLVQPGGSLRLSCAVSGFTFSSYDMNWVR | EIVMTQSPATLSLSPGERA |
| | | | | VQPGGSLRLSC | LSLSPGERAT | QAPGKGLEWVSYISSSSTAKYYADSVKGRFTISRDNAK | TLSCRASQSVSNYLAWYQQ |
| | | | | AVSGFTFSSYD | LSCRASQSVS | NSLYLQMNSLWDEDTAVYYCAREDIVVVTPILQHWGQG | KSGQAPRLLIYDASNRATG |
| | | | | MNWVRQAPGKG | NYLAWYQQKS | TLVTVSSASTKGPSVFPLAPSSKSTSGGTAALGCLVKD | IPARFSGSGSGTDFTLTIS |
| | | | | LEWVSYISSSS | GQAPRLLIYD | YFPEPVTVSWNSGALTSGVHTFPAVLQSSGLYSLSSVV | SLEPEDFAVYCCQQRSNWT |
| | | | | TAKYYADSVKG | ASNRATGIPA | TVPSSSLGTQTYICNVNHKPSNTKVDKKVEPKSCDKTH | FGQGTRLEIKRTVAAPSVF |
| | | | | RFTISRDNAKN | RFSGSGSGTD | TCPPCPAPELLGGPSVFLFPPKPKDTLMISRTPEVTCV | IFPPSDEQLKSGTASVVCL |
| | | | | SLYLQMNSLWD | FTLTISSLEP | VVDVSHEDPEVKFNWYVDGVEVHNAKTKPREEQYNSTY | LNNFYPREAKVQWKVDNAL |
| | | | | EDTAVYYCARE | EDFAVYCCQQ | RVVSVLTVLHQDWLNGKEYKCKVSNKALPAPIEKTISK | QSGNSQESVTEQDSKDSTY |
| | | | | DIVVVTPILQH | RSNWTFGQGT | AKGQPREPQVYTLPPSREEMTKNQVSLTCLVKGFYPSD | SLSSTLTLSKADYEKHKVY |
| | | | | WGQGTLVTVSS | RLEIK | IAVEWESNGQPENNYKTTPPVLDSDGSFFLYSKLTVDK | ACEVTHQGLSSPVTKSFNR |
| | | | | | | SRWQQGNVFSCSVMHEALHNHYTQKSLSLSPGK | GEC |
| | | | | 3261 | 3262 | 3263 | 3264 |
| 97 | VG9B163 | IgG1 | Lambda | QVQLVQSGGGL | QLVLTQPPSV | QVQLVQSGGGLVKPGASLKLSCAASGFTFSNHGMSWVR | QLVLTQPPSVSAAPGQKVT |
| | | | | VKPGASLKLSC | SAAPGQKVTI | QTSDKRLEWVASITRGGDTTYYPDNVKGRFTISRENAK | ISCSGNSSNIGHNYVSWYQ |
| | | | | AASGFTFSNHG | SCSGNSSNIG | NTLYLQMSSLKSDDTALYYCTRGPLTVGYAMDYWGQGT | QLPGTAPKLLIYDNNQRPS |
| | | | | MSWVRQTSDKR | HNYVSWYQQL | TVTVSSASTKGPSVFPLAPSSKSTSGGTAALGCLVKDY | GIPDRFSGSKSDTSATLGI |
| | | | | LEWVASITRGG | PGTAPKLLIY | FPEPVTVSWNSGALTSGVHTFPAVLQSSGLYSLSSVVT | TGLQTGDEADYYCGIWDSS |
| | | | | DTTYYPDNVKG | DNNQRPSGIP | VPSSSLGTQTYICNVNHKPSNTKVDKKVEPKSCDKTHT | LSIVVFGGGTKVTVLGQPK |
| | | | | RFTISRENAKN | DRFSGSKSDT | CPPCPAPELLGGPSVFLFPPKPKDTLMISRTPEVTCVV | AAPSVTLFPPSSEELQANK |
| | | | | TLYLQMSSLKS | SATLGITGLQ | VDVSHEDPEVKFNWYVDGVEVHNAKTKPREEQYNSTYR | ATLVCLISDFYPGAVTVAW |
| | | | | DDTALYYCTRG | TGDEADYYCG | VVSVLTVLHQDWLNGKEYKCKVSNKALPAPIEKTISKA | KADSSPVKAGVETTTPSKQ |
| | | | | PLTVGYAMDYW | IWDSSLSIVV | KGQPREPQVYTLPPSREEMTKNQVSLTCLVKGFYPSDI | SNNKYAASSYLSLTPEQWK |
| | | | | GQGTTVTVSS | FGGGTKVTVL | AVEWESNGQPENNYKTTPPVLDSDGSFFLYSKLTVDKS | SHRSYSCQVTHEGSTVEKT |
| | | | | | | RWQQGNVFSCSVMHEALHNHYTQKSLSLSPGK | VAPTECS |
| | | | | 3295 | 3296 | 3297 | 3298 |
| 98 | VG9B454 | IgG1 | Lambda | QVQLQESGPGL | QAVLTQPASV | QVQLQESGPGLVKPSETLSLTCTVSGGSISNYYWSWIR | QAVLTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3329 | 3330 | 3331 | 3332 |
| 99 | VG9B439 | IgG1 | Lambda | QVQLQQSGPGL | QSALTQPASV | QVQLQQSGPGLVKPSETLSLTCTVSGGSISNYYWSWIR | QSALTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3363 | 3364 | 3365 | 3366 |
| 100 | VG9B68 | IgG1 | Kappa | EVQLVESGAEV | EIVMTQSPLS | EVQLVESGAEVKKPGSSVKVSCKASGGTFSTYAISWVR | EIVMTQSPLSSPVTLGQPA |
| | | | | KKPGSSVKVSC | SPVTLGQPAS | QAPGQGLEWMGGIIPIFGTATYAQRFQDRVMITADKST | SISCRSSQSLVHSDGNTYL |
| | | | | KASGGTFSTYA | ISCRSSQSLV | TTAYMELSSLRSEDTAVFYCARGVGWGSDYYYGLDVWG | SWLQQRPGQSPRLLIYKIS |
| | | | | ISWVRQAPGQG | HSDGNTYLSW | QGTMVTVSSASTKGPSVFPLAPSSKSTSGGTAALGCLV | NRFSGVPDRFSGSGAGTDF |
| | | | | LEWMGGIIPIF | LQQRPGQSPR | KDYFPEPVTVSWNSGALTSGVHTFPAVLQSSGLYSLSS | TLRITRVEPEDVGVYYCIQ |
| | | | | GTATYAQRFQD | LLIYKISNRF | VVTVPSSSLGTQTYICNVNHKPSNTKVDKKVEPKSCDK | ATHHPLTFGGGTRLEIKRT |
| | | | | RVMITADKSTT | SGVPDRFSGS | THTCPPCPAPELLGGPSVFLFPPKPKDTLMISRTPEVT | VAAPSVFIFPPSDEQLKSG |
| | | | | TAYMELSSLRS | GAGTDFTLRI | CVVVDVSHEDPEVKFNWYVDGVEVHNAKTKPREEQYNS | TASVVCLLNNFYPREAKVQ |
| | | | | EDTAVFYCARG | TRVEPEDVGV | TYRVVSVLTVLHQDWLNGKEYKCKVSNKALPAPIEKTI | WKVDNALQSGNSQESVTEQ |
| | | | | VGWGSDYYYGL | YYCIQATHHP | SKAKGQPREPQVYTLPPSREEMTKNQVSLTCLVKGFYP | DSKDSTYSLSSTLTLSKAD |
| | | | | DVWGQGTMVTV | LTFGGGTRLE | SDIAVEWESNGQPENNYKTTPPVLDSDGSFFLYSKLTV | YEKHKVYACEVTHQGLSSP |
| | | | | SS | IK | DKSRWQQGNVFSCSVMHEALHNHYTQKSLSLSPGK | VTKSFNRGEC |
| | | | | 3397 | 3398 | 3399 | 3400 |
| 101 | VG9B449 | IgG1 | Lambda | QVQLQESGPGL | QAVLTQPASV | QVQLQESGPGLVKPSETLSLTCTVSGGSISNYYWSWIR | QAVLTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKVTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKVTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3431 | 3432 | 3433 | 3434 |
| 102 | VG9B204 | IgG1 | Lambda | QVQLQESGPGL | SSELTQPPSV | QVQLQESGPGLVKPSETLSLTCTVSYGSFSSFYWSWIR | SSELTQPPSVSVSPGQTAS |
| | | | | VKPSETLSLTC | SVSPGQTASI | QPAGKGLEWIGRIYTSGGTIYNPSLKSRVTMSVDTSKN | ITCSGDKLGDKYACWYQQK |
| | | | | TVSYGSFSSFY | TCSGDKLGDK | QFSLKLSSVTAADTAVYYCARWLRAFDYWGQGTLVTVS | PGQSPVLVIYQDSKRPSGI |
| | | | | WSWIRQPAGKG | YACWYQQKPG | SASTKGPSVFPLAPSSKSTSGGTAALGCLVKDYFPEPV | PERFSGSNSGNTATLTISG |
| | | | | LEWIGRIYTSG | QSPVLVIYQD | TVSWNSGALTSGVHTFPAVLQSSGLYSLSSVVTVPSSS | TQAMDEADYYCQAWDSSTV |
| | | | | GTIYNPSLKSR | SKRPSGIPER | LGTQTYICNVNHKPSNTKVDKKVEPKSCDKTHTCPPCP | VFGGGTKLTVLGQPKAAPS |
| | | | | VTMSVDTSKNQ | FSGSNSGNTA | APELLGGPSVFLFPPKPKDTLMISRTPEVTCVVVDVSH | VTLFPPSSEELQANKATLV |
| | | | | FSLKLSSVTAA | TLTISGTQAM | EDPEVKFNWYVDGVEVHNAKTKPREEQYNSTYRVVSVL | CLISDFYPGAVTVAWKADS |
| | | | | DTAVYYCARWL | DEADYYCQAW | TVLHQDWLNGKEYKCKVSNKALPAPIEKTISKAKGQPR | SPVKAGVETTTPSKQSNNK |
| | | | | RAFDYWGQGTL | DSSTVVFGGG | EPQVYTLPPSREEMTKNQVSLTCLVKGFYPSDIAVEWE | YAASSYLSLTPEQWKSHRS |
| | | | | VTVSS | TKLTVL | SNGQPENNYKTTPPVLDSDGSFFLYSKLTVDKSRWQQG | YSCQVTHEGSTVEKTVAPT |
| | | | | | | NVFSCSVMHEALHNHYTQKSLSLSPGK | ECS |
| | | | | 3465 | 3466 | 3467 | 3468 |
| 103 | VG9B459 | IgG1 | Lambda | QVQLQESGPGL | QPVLTQPASV | QVQLQESGPGLVKPSETLSLTCTVSGGSISNYYWSWIR | QPVLTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3499 | 3500 | 3501 | 3502 |
| 104 | VG9B157 | IgG1 | Lambda | EVQLLESGGGL | SSELTQDPAV | EVQLLESGGGLVKPGASLKLSCAASGFTFSNHGMSWVR | SSELTQDPAVSVALGQTVR |
| | | | | VKPGASLKLSC | SVALGQTVRI | QTSDKRLEWVASITRGGDTTYYPDNVKGRFTISRENAK | ITCQGDSLRSYYATWYQQK |
| | | | | AASGFTFSNHG | TCQGDSLRSY | NTLYLQMSSLKSDDTALYYCTRGPLTVGYAMDYWGQGT | PGQAPILVIYGENNRPSGI |
| | | | | MSWVRQTSDKR | YATWYQQKPG | TVTVSSASTKGPSVFPLAPSSKSTSGGTAALGCLVKDY | PDRFSGSRSGNTASLTITG |
| | | | | LEWVASITRGG | QAPILVIYGE | FPEPVTVSWNSGALTSGVHTFPAVLQSSGLYSLSSVVT | AQAEDEADYYCNSRDTGDH |
| | | | | DTTYYPDNVKG | NNRPSGIPDR | VPSSSLGTQTYICNVNHKPSNTKVDKKVEPKSCDKTHT | HLVFGGGTKLTVLGQPKAA |
| | | | | RFTISRENAKN | FSGSRSGNTA | CPPCPAPELLGGPSVFLFPPKPKDTLMISRTPEVTCVV | PSVTLFPPSSEELQANKAT |
| | | | | TLYLQMSSLKS | SLTITGAQAE | VDVSHEDPEVKFNWYVDGVEVHNAKTKPREEQYNSTYR | LVCLISDFYPGAVTVAWKA |
| | | | | DDTALYYCTRG | DEADYYCNSR | VVSVLTVLHQDWLNGKEYKCKVSNKALPAPIEKTISKA | DSSPVKAGVETTTPSKQSN |
| | | | | PLTVGYAMDYW | DTGDHHLVFG | KGQPREPQVYTLPPSREEMTKNQVSLTCLVKGFYPSDI | NKYAASSYLSLTPEQWKSH |
| | | | | GQGTTVTVSS | GGTKLTVL | AVEWESNGQPENNYKTTPPVLDSDGSFFLYSKLTVDKS | RSYSCQVTHEGSTVEKTVA |
| | | | | | | RWQQGNVFSCSVMHEALHNHYTQKSLSLSPGK | PTECS |
| | | | | 3533 | 3534 | 3535 | 3536 |
| 105 | VG9B453 | IgG1 | Lambda | QVQLQQSGPGL | QSALTQPASV | QVQLQQSGPGLVKPSETLSLTCTVSGGSISNYYWSWIR | QSALTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKVTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKVTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3567 | 3568 | 3569 | 3570 |
| 106 | VG9B443 | IgG1 | Lambda | QVQLQESGPGL | QSALTQPASV | QVQLQESGPGLVKPSETLSLTCTVSGGSISNYYWSWIR | QSALTQPASVSGSPGQSIT |
| | | | | TVSGGSISNYY | SCTGTSSDVG | QPAGKGLGWIGRIYSSGITNYNPSLKSRVTMSVDTSKN | ISCTGTSSDVGGYNYVSWY |
| | | | | WSWIRQPAGKG | GYNYVSWYQQ | QLSLKLSSVTAADTAVYYCAGIVGVKGAFAIWGQGTMV | QQHPGKAPKLMIYEVSNRP |
| | | | | LGWIGRIYSSG | HPGKAPKLMI | TVSSASTKGPSVFPLAPSSKSTSGGTAALGCLVKDYFP | SGVSNRFSGSKSGNTASLT |
| | | | | ITNYNPSLKSR | YEVSNRPSGV | EPVTVSWNSGALTSGVHTFPAVLQSSGLYSLSSVVTVP | ISGLQAEDEADYYCSSYTS |
| | | | | VTMSVDTSKNQ | SNRFSGSKSG | SSSLGTQTYICNVNHKPSNTKVDKKVEPKSCDKTHTCP | TSVVFGGGTKLTVLGQPKA |
| | | | | LSLKLSSVTAA | NTASLTISGL | PCPAPELLGGPSVFLFPPKPKDTLMISRTPEVTCVVVD | APSVTLFPPSSEELQANKA |
| | | | | DTAVYYCAGIV | QAEDEADYYC | VSHEDPEVKFNWYVDGVEVHNAKTKPREEQYNSTYRVV | TLVCLISDFYPGAVTVAWK |
| | | | | GVKGAFAIWGQ | SSYTSTSVVF | SVLTVLHQDWLNGKEYKCKVSNKALPAPIEKTISKAKG | ADSSPVKAGVETTTPSKQS |
| | | | | GTMVTVSS | GGGTKLTVL | QPREPQVYTLPPSREEMTKNQVSLTCLVKGFYPSDIAV | NNKYAASSYLSLTPEQWKS |
| | | | | | | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3601 | 3602 | 3603 | 3604 |
| 107 | VG9B455 | IgG1 | Lambda | QVQLVESGPGL | SYELTQPASV | QVQLVESGPGLVKPSETLSLTCTVSGGSISNYYWSWIR | SYELTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3635 | 3636 | 3637 | 3638 |
| 108 | VG9B466 | IgG1 | Lambda | QVQLVQSGPGL | SYELTQPASV | QVQLVQSGPGLVKPSETLSLTCTVSGGSISNYYWSWIR | SYELTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3669 | 3670 | 3671 | 3672 |
| 109 | VG9B450 | IgG1 | Lambda | EVQLQESGPGL | QSVLTQPASV | EVQLQESGPGLVKPSETLSLTCTVSGGSISNYYWSWIR | QSVLTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3703 | 3704 | 3705 | 3706 |
| 110 | VG9B438 | IgG1 | Lambda | EVQLVESGPGL | SYELTQPASV | EVQLVESGPGLVKPSETLSLTCTVSGGSISNYYWSWIR | SYELTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3737 | 3738 | 3739 | 3740 |
| 111 | VG9B464 | IgG1 | Lambda | EVQLVQSGPGL | QSALTQPASV | EVQLVQSGPGLVKPSETLSLTCTVSGGSISNYYWSWIR | QSALTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTTV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTTVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3771 | 3772 | 3773 | 3774 |
| 112 | VG9B437 | IgG1 | Lambda | EVQLQESGPGL | QSALTQPASV | EVQLQESGPGLVKPSETLSLTCTVSGGSISNYYWSWIR | QSALTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3805 | 3806 | 3807 | 3808 |
| 113 | VG9B457 | IgG1 | Lambda | EVQLVESGPGL | QAVLTQPASV | EVQLVESGPGLVKPSETLSLTCTVSGGSISNYYWSWIR | QAVLTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3839 | 3840 | 3841 | 3842 |
| 114 | VG9B442 | IgG1 | Lambda | QVQLVESGPGL | QSALTQPASV | QVQLVESGPGLVKPSETLSLTCTVSGGSISNYYWSWIR | QSALTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKVTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKVTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3873 | 3874 | 3875 | 3876 |
| 115 | VG9B436 | IgG1 | Lambda | EVQLVESGPGL | QSVLTQPASV | EVQLVESGPGLVKPSETLSLTCTVSGGSISNYYWSWIR | QSVLTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3907 | 3908 | 3909 | 3910 |
| 116 | VG9B434 | IgG1 | Lambda | QVQLQESGPGL | QSVLTQPASV | QVQLQESGPGLVKPSETLSLTCTVSGGSISNYYWSWIR | QSVLTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3941 | 3942 | 3943 | 3944 |
| 117 | VG9B460 | IgG1 | Lambda | EVQLVESGPGL | QLVLTQPASV | EVQLVESGPGLVKPSETLSLTCTVSGGSISNYYWSWIR | QLVLTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 3975 | 3976 | 3977 | 3978 |
| 118 | VG9B189 | IgG1 | Lambda | EVQLVQSGGGL | QSVVTQPPSA | EVQLVQSGGGLVKPGASLKLSCAASGFTFSNHGMSWVR | QSVVTQPPSASGSPGQSVT |
| | | | | VKPGASLKLSC | SGSPGQSVTI | QTSDKRLEWVASITRGGDTTYYPDNVKGRFTISRENAK | ISCTGTSSDVGGYNYVSWY |
| | | | | AASGFTFSNHG | SCTGTSSDVG | NTLYLQMSSLKSDDTALYYCTRGPLTVGYAMDYWGQGT | QQHPGKAPKLMIYEVSKRP |
| | | | | MSWVRQTSDKR | GYNYVSWYQQ | TVTVSSASTKGPSVFPLAPSSKSTSGGTAALGCLVKDY | SGVPDRFSGSKSGNTASLT |
| | | | | LEWVASITRGG | HPGKAPKLMI | FPEPVTVSWNSGALTSGVHTFPAVLQSSGLYSLSSVVT | VSGLQAEDEADYYCNSYAG |
| | | | | DTTYYPDNVKG | YEVSKRPSGV | VPSSSLGTQTYICNVNHKPSNTKVDKKVEPKSCDKTHT | SNNFEVFGGGTKVTVLGQP |
| | | | | RFTISRENAKN | PDRFSGSKSG | CPPCPAPELLGGPSVFLFPPKPKDTLMISRTPEVTCVV | KAAPSVTLFPPSSEELQAN |
| | | | | TLYLQMSSLKS | NTASLTVSGL | VDVSHEDPEVKFNWYVDGVEVHNAKTKPREEQYNSTYR | KATLVCLISDFYPGAVTVA |
| | | | | DDTALYYCTRG | QAEDEADYYC | VVSVLTVLHQDWLNGKEYKCKVSNKALPAPIEKTISKA | WKADSSPVKAGVETTTPSK |
| | | | | PLTVGYAMDYW | NSYAGSNNFE | KGQPREPQVYTLPPSREEMTKNQVSLTCLVKGFYPSDI | QSNNKYAASSYLSLTPEQW |
| | | | | GQGTTVTVSS | VFGGGTKVTV | AVEWESNGQPENNYKTTPPVLDSDGSFFLYSKLTVDKS | KSHRSYSCQVTHEGSTVEK |
| | | | | | L | RWQQGNVFSCSVMHEALHNHYTQKSLSLSPGK | TVAPTECS |
| | | | | 4009 | 4010 | 4011 | 4012 |
| 119 | VG9B452 | IgG1 | Lambda | QVQLVESGPGL | QSALTQPASV | VQLVESGPGLVKPSETLSLTCTVSGGSISNYYWSWIR | QSALTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 4043 | 4044 | 4045 | 4046 |
| 120 | VG9B53 | IgG1 | Kappa | QVQLQESGPGL | DIQMTQSPLS | QVQLQESGPGLVKPSETLSLTCTVSGGSISSYYWSWIR | DIQMTQSPLSLPVTPGEPA |
| | | | | VKPSETLSLTC | LPVTPGEPAS | QPAGKGLEWIGRIHTIGSINYNPSLKSRVTMSVDTSKN | SISCRSSQSLLHSNGYNYL |
| | | | | TVSGGSISSYY | ISCRSSQSLL | QFSLKLSSVTAADTAVYYCAMEGVGATNYYYGMAVWGR | DWYLQKPGQSPQLLIYLGS |
| | | | | WSWIRQPAGKG | HSNGYNYLDW | GTMVTVSSASTKGPSVFPLAPSSKSTSGGTAALGCLVK | NRASGVPDRFSGSGSGTDF |
| | | | | LEWIGRIHTIG | YLQKPGQSPQ | DYFPEPVTVSWNSGALTSGVHTFPAVLQSSGLYSLSSV | TLKISRVEAEDVGVYYCMQ |
| | | | | SINYNPSLKSR | LLIYLGSNRA | VTVPSSSLGTQTYICNVNHKPSNTKVDKKVEPKSCDKT | ALQTPYTFGQGTKVEIKRT |
| | | | | VTMSVDTSKNQ | SGVPDRFSGS | HTCPPCPAPELLGGPSVFLFPPKPKDTLMISRTPEVTC | VAAPSVFIFPPSDEQLKSG |
| | | | | FSLKLSSVTAA | GSGTDFTLKI | VVVDVSHEDPEVKFNWYVDGVEVHNAKTKPREEQYNST | TASVVCLLNNFYPREAKVQ |
| | | | | DTAVYYCAMEG | SRVEAEDVGV | YRVVSVLTVLHQDWLNGKEYKCKVSNKALPAPIEKTIS | WKVDNALQSGNSQESVTEQ |
| | | | | VGATNYYYGMA | YYCMQALQTP | KAKGQPREPQVYTLPPSREEMTKNQVSLTCLVKGFYPS | DSKDSTYSLSSTLTLSKAD |
| | | | | VWGRGTMVTVS | YTFGQGTKVE | DIAVEWESNGQPENNYKTTPPVLDSDGSFFLYSKLTVD | YEKHKVYACEVTHQGLSSP |
| | | | | S | IK | KSRWQQGNVFSCSVMHEALHNHYTQKSLSLSPGK | VTKSFNRGEC |
| | | | | 4077 | 4078 | 4079 | 4080 |
| 121 | VG9B41 | IgG1 | Kappa | QVQLQESGPQL | EIVLTQTPLS | QVQLQESGPQLVRPGASVKISCKASGYSFTTYWMHWVK | EIVLTQTPLSSPVTLGQPA |
| | | | | VRPGASVKISC | SPVTLGQPAS | QRPGQGLEWIGLIDPSDSETRLNQKFKDKATLTVDKSS | SISCRSSQSLVHSDGNTYL |
| | | | | KASGYSFTTYW | ISCRSSQSLV | STAYMRLSSPTSEDSAVYYCASNRWLLGWGQGTMVTVS | SWLHQRPGQPPRLLIYKIS |
| | | | | MHWVKQRPGQG | HSDGNTYLSW | SASTKGPSVFPLAPSSKSTSGGTAALGCLVKDYFPEPV | NRFSGVPDRFSGSGAGTDF |
| | | | | LEWIGLIDPSD | LHQRPGQPPR | TVSWNSGALTSGVHTFPAVLQSSGLYSLSSVVTVPSSS | TLKISRVETEDVGVYYCVQ |
| | | | | SETRLNQKFKD | LLIYKISNRF | LGTQTYICNVNHKPSNTKVDKKVEPKSCDKTHTCPPCP | TREFPLTFGGGTKVEIKRT |
| | | | | KATLTVDKSSS | SGVPDRFSGS | APELLGGPSVFLFPPKPKDTLMISRTPEVTCVVVDVSH | VAAPSVFIFPPSDEQLKSG |
| | | | | TAYMRLSSPTS | GAGTDFTLKI | EDPEVKFNWYVDGVEVHNAKTKPREEQYNSTYRVVSVL | TASVVCLLNNFYPREAKVQ |
| | | | | EDSAVYYCASN | SRVETEDVGV | TVLHQDWLNGKEYKCKVSNKALPAPIEKTISKAKGQPR | WKVDNALQSGNSQESVTEQ |
| | | | | RWLLGWGQGTM | YYCVQTREFP | EPQVYTLPPSREEMTKNQVSLTCLVKGFYPSDIAVEWE | DSKDSTYSLSSTLTLSKAD |
| | | | | VTVSS | LTFGGGTKVE | SNGQPENNYKTTPPVLDSDGSFFLYSKLTVDKSRWQQG | YEKHKVYACEVTHQGLSSP |
| | | | | | IK | NVFSCSVMHEALHNHYTQKSLSLSPGK | VTKSFNRGEC |
| | | | | 4111 | 4112 | 4113 | 4114 |
| 122 | VG9B136 | IgG1 | Kappa | QVQLQQSGPGL | DIVMTQSPSF | QVQLQQSGPGLVKPSQTLSLTCAISGDSVSSNRAAWNW | DIVMTQSPSFLSTFVGDRV |
| | | | | VKPSQTLSLTC | LSTFVGDRVT | IRQSPSKGLEWLGRTYYRSKWYNEYAASVKSRMSINPD | TITCRASQGISSYLAWYQQ |
| | | | | AISGDSVSSNR | ITCRASQGIS | TSKNQFSLQLNSVTPEDTALYYCARDLWELREACDIWG | KPGKVPKLLIYVASTLQSG |
| | | | | AAWNWIRQSPS | SYLAWYQQKP | QGTMVTVSSASTKGPSVFPLAPSSKSTSGGTAALGCLV | VPSRFSGSGSGTEFTLTIS |
| | | | | KGLEWLGRTYY | GKVPKLLIYV | KDYFPEPVTVSWNSGALTSGVHTFPAVLQSSGLYSLSS | SLQPEDFATYYCQQLNSYP |
| | | | | RSKWYNEYAAS | ASTLQSGVPS | VVTVPSSSLGTQTYICNVNHKPSNTKVDKKVEPKSCDK | FTFGPGTKVDIKRTVAAPS |
| | | | | VKSRMSINPDT | RFSGSGSGTE | THTCPPCPAPELLGGPSVFLFPPKPKDTLMISRTPEVT | VFIFPPSDEQLKSGTASVV |
| | | | | SKNQFSLQLNS | FTLTISSLQP | CVVVDVSHEDPEVKFNWYVDGVEVHNAKTKPREEQYNS | CLLNNFYPREAKVQWKVDN |
| | | | | VTPEDTALYYC | EDFATYYCQQ | TYRVVSVLTVLHQDWLNGKEYKCKVSNKALPAPIEKTI | ALQSGNSQESVTEQDSKDS |
| | | | | ARDLWELREAC | LNSYPFTFGP | SKAKGQPREPQVYTLPPSREEMTKNQVSLTCLVKGFYP | TYSLSSTLTLSKADYEKHK |
| | | | | DIWGQGTMVTV | GTKVDIK | SDIAVEWESNGQPENNYKTTPPVLDSDGSFFLYSKLTV | VYACEVTHQGLSSPVTKSF |
| | | | | SS | | DKSRWQQGNVFSCSVMHEALHNHYTQKSLSLSPGK | NRGEC |
| | | | | 4145 | 4146 | 4147 | 4148 |
| 123 | VG9B399 | IgG1 | Kappa | EVQLVESGGGL | ETTLTQSPGT | EVQLVESGGGLVKPGGSLRLSCSASGFTFSNYDMNWVR | ETTLTQSPGTLSLSPGDRA |
| | | | | VKPGGSLRLSC | LSLSPGDRAT | QAPGKGLEWVSSISSSSSYIYYADSVKGRFTISRDNAK | TLSCRASQSVASSYLAWYQ |
| | | | | SASGFTFSNYD | LSCRASQSVA | NSLYLQMNSLRAEDTAVYHCARDVGVTTDYYYYGMDVW | QKPGQSPRLLIYGASSRAT |
| | | | | MNWVRQAPGKG | SSYLAWYQQK | GQGTMVTVSSASTKGPSVFPLAPSSKSTSGGTAALGCL | GIPDRFSGSGSGTDFTLTI |
| | | | | LEWVSSISSSS | PGQSPRLLIY | VKDYFPEPVTVSWNSGALTSGVHTFPAVLQSSGLYSLS | SRLEPEDFAVYYCQQYGSS |
| | | | | SYIYYADSVKG | GASSRATGIP | SVVTVPSSSLGTQTYICNVNHKPSNTKVDKKVEPKSCD | PPYTFGQGTKVDIKRTVAA |
| | | | | RFTISRDNAKN | DRFSGSGSGT | KTHTCPPCPAPELLGGPSVFLFPPKPKDTLMISRTPEV | PSVFIFPPSDEQLKSGTAS |
| | | | | SLYLQMNSLRA | DFTLTISRLE | TCVVVDVSHEDPEVKFNWYVDGVEVHNAKTKPREEQYN | VVCLLNNFYPREAKVQWKV |
| | | | | EDTAVYHCARD | PEDFAVYYCQ | STYRVVSVLTVLHQDWLNGKEYKCKVSNKALPAPIEKT | DNALQSGNSQESVTEQDSK |
| | | | | VGVTTDYYYYG | QYGSSPPYTF | ISKAKGQPREPQVYTLPPSREEMTKNQVSLTCLVKGFY | DSTYSLSSTLTLSKADYEK |
| | | | | MDVWGQGTMVT | GQGTKVDIK | PSDIAVEWESNGQPENNYKTTPPVLDSDGSFFLYSKLT | HKVYACEVTHQGLSSPVTK |
| | | | | VSS | | VDKSRWQQGNVFSCSVMHEALHNHYTQKSLSLSPGK | SFNRGEC |
| | | | | 4179 | 4180 | 4181 | 4182 |
| 124 | VG9B369 | IgG1 | Kappa | EVQLLESGPTL | DIVMTQSPSS | EVQLLESGPTLVKPTQTLTLTCTFSGFSLSTIGVGVGW | DIVMTQSPSSLSASVGDRV |
| | | | | VKPTQTLTLTC | LSASVGDRVT | IRQPPGKALEWLSLIYWNDDKRYNPSLKSRLTITKDTS | TITCQASQDISHYLNWYQQ |
| | | | | TFSGFSLSTIG | ITCQASQDIS | KNQVVLTMTNMDPVDTATYYCAHSHDWVHAFDIWGQGT | KPGKAPKFLIYDASNLDTG |
| | | | | VGVGWIRQPPG | HYLNWYQQKP | MVTVSSASTKGPSVFPLAPSSKSTSGGTAALGCLVKDY | VPSRFSGSGSGTDFTFTIS |
| | | | | KALEWLSLIYW | GKAPKFLIYD | FPEPVTVSWNSGALTSGVHTFPAVLQSSGLYSLSSVVT | SLQPEDIATYYCQQYDNFP |
| | | | | NDDKRYNPSLK | ASNLDTGVPS | VPSSSLGTQTYICNVNHKPSNTKVDKKVEPKSCDKTHT | LTFGGGTKVEIKRTVAAPS |
| | | | | SRLTITKDTSK | RFSGSGSGTD | CPPCPAPELLGGPSVFLFPPKPKDTLMISRTPEVTCVV | VFIFPPSDEQLKSGTASVV |
| | | | | NQVVLTMTNMD | FTFTISSLQP | VDVSHEDPEVKFNWYVDGVEVHNAKTKPREEQYNSTYR | CLLNNFYPREAKVQWKVDN |
| | | | | PVDTATYYCAH | EDIATYYCQQ | VVSVLTVLHQDWLNGKEYKCKVSNKALPAPIEKTISKA | ALQSGNSQESVTEQDSKDS |
| | | | | SHDWVHAFDIW | YDNFPLTFGG | KGQPREPQVYTLPPSREEMTKNQVSLTCLVKGFYPSDI | TYSLSSTLTLSKADYEKHK |
| | | | | GQGTMVTVSS | GTKVEIK | AVEWESNGQPENNYKTTPPVLDSDGSFFLYSKLTVDKS | VYACEVTHQGLSSPVTKSF |
| | | | | | | RWQQGNVFSCSVMHEALHNHYTQKSLSLSPGK | NRGEC |
| | | | | 4213 | 4214 | 4215 | 4216 |
| 125 | VG9B144 | IgG1 | Lambda | QVQLVQSGAEV | SSELTQDPAV | QVQLVQSGAEVKKPGASVKVSCKASGYTFTGYYMHWMR | SSELTQDPAVSVALGQTVR |
| | | | | KKPGASVKVSC | SVALGQTVRI | QAPGQGLEWMGWINPNSGATNKEQKFQGRVTMTRDTSI | ITCQGDNLRNYYATWFQQK |
| | | | | KASGYTFTGYY | TCQGDNLRNY | STAYMELSRLRSDDTAVYYCAREDDAFDVWGQGTMVTV | PGQAPVLVFFGKNNRPSGI |
| | | | | MHWMRQAPGQG | YATWFQQKPG | SSASTKGPSVFPLAPSSKSTSGGTAALGCLVKDYFPEP | PDRFSGSSSGDTASLTITG |
| | | | | LEWMGWINPNS | QAPVLVFFGK | VTVSWNSGALTSGVHTFPAVLQSSGLYSLSSVVTVPSS | AQAEDEADYYCNSRDSSGN |
| | | | | GATNKEQKFQG | NNRPSGIPDR | SLGTQTYICNVNHKPSNTKVDKKVEPKSCDKTHTCPPC | HVVFGGGTKLTVLGQPKAA |
| | | | | RVTMTRDTSIS | FSGSSSGDTA | PAPELLGGPSVFLFPPKPKDTLMISRTPEVTCVVVDVS | PSVTLFPPSSEELQANKAT |
| | | | | TAYMELSRLRS | SLTITGAQAE | HEDPEVKFNWYVDGVEVHNAKTKPREEQYNSTYRVVSV | LVCLISDFYPGAVTVAWKA |
| | | | | DDTAVYYCARE | DEADYYCNSR | LTVLHQDWLNGKEYKCKVSNKALPAPIEKTISKAKGQP | DSSPVKAGVETTTPSKQSN |
| | | | | DDAFDVWGQGT | DSSGNHVVFG | REPQVYTLPPSREEMTKNQVSLTCLVKGFYPSDIAVEW | NKYAASSYLSLTPEQWKSH |
| | | | | MVTVSS | GGTKLTVL | ESNGQPENNYKTTPPVLDSDGSFFLYSKLTVDKSRWQQ | RSYSCQVTHEGSTVEKTVA |
| | | | | | | GNVFSCSVMHEALHNHYTQKSLSLSPGK | PTECS |
| | | | | 4247 | 4248 | 4249 | 4250 |
| 126 | VG9B410 | IgG1 | Kappa | EVQLVESGGGL | DIQMTQSPSS | EVQLVESGGGLVKPGGSLRLSCAASGFTFSNYGMNWVR | DIQMTQSPSSVSASVGDRV |
| | | | | VKPGGSLRLSC | VSASVGDRVT | QAPGKGLEWVSYISSGSSYKYYADSMKGRFTISRDNAM | TITCRASQGINSWLAWYQQ |
| | | | | AASGFTFSNYG | ITCRASQGIN | NLLYLQMNSLRPEDSAMYYCARDPVVTEYYYYGMDVWG | KPGNAPKLLIYAASSLQSG |
| | | | | MNWVRQAPGKG | SWLAWYQQKP | QGTTVTVSSASTKGPSVFPLAPSSKSTSGGTAALGCLV | VPSRFSGIGSGTDFIFTIS |
| | | | | LEWVSYISSGS | GNAPKLLIYA | KDYFPEPVTVSWNSGALTSGVHTFPAVLQSSGLYSLSS | SLQPEDFASYYCQQANSFP |
| | | | | SYKYYADSMKG | ASSLQSGVPS | VVTVPSSSLGTQTYICNVNHKPSNTKVDKKVEPKSCDK | WTFGQGTKVEIKRTVAAPS |
| | | | | RFTISRDNAMN | RFSGIGSGTD | THTCPPCPAPELLGGPSVFLFPPKPKDTLMISRTPEVT | VFIFPPSDEQLKSGTASVV |
| | | | | LLYLQMNSLRP | FIFTISSLQP | CVVVDVSHEDPEVKFNWYVDGVEVHNAKTKPREEQYNS | CLLNNFYPREAKVQWKVDN |
| | | | | EDSAMYYCARD | EDFASYYCQQ | TYRVVSVLTVLHQDWLNGKEYKCKVSNKALPAPIEKTI | ALQSGNSQESVTEQDSKDS |
| | | | | PVVTEYYYYGM | ANSFPWTFGQ | SKAKGQPREPQVYTLPPSREEMTKNQVSLTCLVKGFYP | TYSLSSTLTLSKADYEKHK |
| | | | | DVWGQGTTVTV | GTKVEIK | SDIAVEWESNGQPENNYKTTPPVLDSDGSFFLYSKLTV | VYACEVTHQGLSSPVTKSF |
| | | | | SS | | DKSRWQQGNVFSCSVMHEALHNHYTQKSLSLSPGK | NRGEC |
| | | | | 4281 | 4282 | 4283 | 4284 |
| 127 | VG9B386 | IgG1 | Kappa | EVQLVESGGGL | EIVMTQSPGT | EVQLVESGGGLVKPGGSLRLSCAASGFTFSSYSMIWVR | EIVMTQSPGTLSLSPGERA |
| | | | | VKPGGSLRLSC | LSLSPGERAT | QAPGKGLEWVSSIGSSSTYIYYADSVRGRFTISRDNAK | TLSCRASQSISRSYLAWYQ |
| | | | | AASGFTFSSYS | LSCRASQSIS | TSLYLQMNSLRAEDTAMYFCARDGETGGFDYWGQGILV | QKPGQPPRLLIYGPSNRAT |
| | | | | MIWVRQAPGKG | RSYLAWYQQK | TVSSASTKGPSVFPLAPSSKSTSGGTAALGCLVKDYFP | GIPDRFSASGSGTDFTLTN |
| | | | | LEWVSSIGSSS | PGQPPRLLIY | EPVTVSWNSGALTSGVHTFPAVLQSSGLYSLSSVVTVP | SRLEPEDFAVYYCQQYGSL |
| | | | | TYIYYADSVRG | GPSNRATGIP | SSSLGTQTYICNVNHKPSNTKVDKKVEPKSCDKTHTCP | PLTFGGGTKVEIKRTVAAP |
| | | | | RFTISRDNAKT | DRFSASGSGT | PCPAPELLGGPSVFLFPPKPKDTLMISRTPEVTCVVVD | SVFIFPPSDEQLKSGTASV |
| | | | | SLYLQMNSLRA | DFTLTNSRLE | VSHEDPEVKFNWYVDGVEVHNAKTKPREEQYNSTYRVV | VCLLNNFYPREAKVQWKVD |
| | | | | EDTAMYFCARD | PEDFAVYYCQ | SVLTVLHQDWLNGKEYKCKVSNKALPAPIEKTISKAKG | NALQSGNSQESVTEQDSKD |
| | | | | GETGGFDYWGQ | QYGSLPLTFG | QPREPQVYTLPPSREEMTKNQVSLTCLVKGFYPSDIAV | STYSLSSTLTLSKADYEKH |
| | | | | GILVTVSS | GGTKVEIK | EWESNGQPENNYKTTPPVLDSDGSFFLYSKLTVDKSRW | KVYACEVTHQGLSSPVTKS |
| | | | | | | QQGNVFSCSVMHEALHNHYTQKSLSLSPGK | FNRGEC |
| | | | | 4315 | 4316 | 4317 | 4318 |
| 128 | VG9B125 | IgG1 | Kappa | EVQLVESGGTL | EIVMTQSPAT | EVQLVESGGTLVQPGGSLRLSCAVSGFTFSSYDMNWVR | EIVMTQSPATLSLSPGERA |
| | | | | VQPGGSLRLSC | LSLSPGERAI | QAPGKGLEWVSYISSSSTAKYYADSVKGRFTISRDNAK | ILSCRASQSVSKDLAWYQQ |
| | | | | AVSGFTFSSYD | LSCRASQSVS | NSLYLQMNSLWDEDTAVYYCAREDIVVVTPILQHWGQG | KPGQAPRLLIYDASNRATG |
| | | | | MNWVRQAPGKG | KDLAWYQQKP | TTVTVSSASTKGPSVFPLAPSSKSTSGGTAALGCLVKD | IPARISGSGSGTDFTLTIS |
| | | | | LEWVSYISSSS | GQAPRLLIYD | YFPEPVTVSWNSGALTSGVHTFPAVLQSSGLYSLSSW | SLEPEDFAVYYCQQRINWP |
| | | | | TAKYYADSVKG | ASNRATGIPA | TVPSSSLGTQTYICNVNHKPSNTKVDKKVEPKSCDKTH | LFTFGPGTKVEIKRTVAAP |
| | | | | RFTISRDNAKN | RISGSGSGTD | TCPPCPAPELLGGPSVFLFPPKPKDTLMISRTPEVTCV | SVFIFPPSDEQLKSGTASV |
| | | | | SLYLQMNSLWD | FTLTISSLEP | VVDVSHEDPEVKFNWYVDGVEVHNAKTKPREEQYNSTY | VCLLNNFYPREAKVQWKVD |
| | | | | EDTAVYYCARE | EDFAVYYCQQ | RVVSVLTVLHQDWLNGKEYKCKVSNKALPAPIEKTISK | NALQSGNSQESVTEQDSKD |
| | | | | DIVVVTPILQH | RINWPLFTFG | AKGQPREPQVYTLPPSREEMTKNQVSLTCLVKGFYPSD | STYSLSSTLTLSKADYEKH |
| | | | | WGQGTTVTVSS | PGTKVEIK | IAVEWESNGQPENNYKTTPPVLDSDGSFFLYSKLTVDK | KVYACEVTHQGLSSPVTKS |
| | | | | | | SRWQQGNVFSCSVMHEALHNHYTQKSLSLSPGK | FNRGEC |
| | | | | 4349 | 4350 | 4351 | 4352 |
| 129 | VG9B112 | IgG1 | Kappa | QVQLQQSGPGL | DIQMTQSPSF | QVQLQQSGPGLVKPSQTLSLTCAISGDSVSSNSAAWNW | DIQMTQSPSFLSASVGDRV |
| | | | | VKPSQTLSLTC | LSASVGDRVI | IRQSPSRGLEWLGGTYYRSKWFNNYAVSVKSRITINPD | IITCRASQGIHSYLAWYQQ |
| | | | | AISGDSVSSNS | ITCRASQGIH | TSTNQFSLQLNSVTPADTAVYYCARGEWGLRDAFDIWG | KPGKVPKLLIYVASTLQSG |
| | | | | AAWNWIRQSPS | SYLAWYQQKP | QGTMVTVSSASTKGPSVFPLAPSSKSTSGGTAALGCLV | VPSRFSGSGSGTEFTLTIS |
| | | | | RGLEWLGGTYY | GKVPKLLIYV | KDYFPEPVTVSWNSGALTSGVHTFPAVLQSSGLYSLSS | SLQPEDFATYYCQQLNSYP |
| | | | | RSKWFNNYAVS | ASTLQSGVPS | VVTVPSSSLGTQTYICNVNHKPSNTKVDKKVEPKSCDK | WTFGQGTKLEIKRTVAAPS |
| | | | | VKSRITINPDT | RFSGSGSGTE | THTCPPCPAPELLGGPSVFLFPPKPKDTLMISRTPEVT | VFIFPPSDEQLKSGTASVV |
| | | | | STNQFSLQLNS | FTLTISSLQP | CVVVDVSHEDPEVKFNWYVDGVEVHNAKTKPREEQYNS | CLLNNFYPREAKVQWKVDN |
| | | | | VTPADTAVYYC | EDFATYYCQQ | TYRVVSVLTVLHQDWLNGKEYKCKVSNKALPAPIEKTI | ALQSGNSQESVTEQDSKDS |
| | | | | ARGEWGLRDAF | LNSYPWTFGQ | SKAKGQPREPQVYTLPPSREEMTKNQVSLTCLVKGFYP | TYSLSSTLTLSKADYEKHK |
| | | | | DIWGQGTMVTV | GTKLEIK | SDIAVEWESNGQPENNYKTTPPVLDSDGSFFLYSKLTV | VYACEVTHQGLSSPVTKSF |
| | | | | SS | | DKSRWQQGNVFSCSVMHEALHNHYTQKSLSLSPGK | NRGEC |
| | | | | 4383 | 4384 | 4385 | 4386 |
| 130 | VG9B371 | IgG1 | Kappa | EVQLLESGGGL | EIVMTQSPGT | EVQLLESGGGLVKPGGSLRLSCAASRFTLSSYDMNWVR | EIVMTQSPGTLSLSPGERA |
| | | | | VKPGGSLRLSC | LSLSPGERAT | QAPGKGLEWVSSISSSSSYIYYADSVKGRFTISRDNAK | TLSCRASQSVSSSYLAWYQ |
| | | | | AASRFTLSSYD | LSCRASQSVS | NSLYLQMNSLRAEDTAVYYCARDRGVGGTDYYYYGLDV | QKPGQAPRLLIYGASSRAT |
| | | | | MNWVRQAPGKG | SSYLAWYQQK | WGQGTTVTVSSASTKGPSVFPLAPSSKSTSGGTAALGC | GIPDRFSGSGSGTDFTLTI |
| | | | | LEWVSSISSSS | PGQAPRLLIY | LVKDYFPEPVTVSWNSGALTSGVHTFPAVLQSSGLYSL | SRLEPEDFAVYYCQQYGSS |
| | | | | SYIYYADSVKG | GASSRATGIP | SSVVTVPSSSLGTQTYICNVNHKPSNTKVDKKVEPKSC | PPYTFGQGTKVEIKRTVAA |
| | | | | RFTISRDNAKN | DRFSGSGSGT | DKTHTCPPCPAPELLGGPSVFLFPPKPKDTLMISRTPE | PSVFIFPPSDEQLKSGTAS |
| | | | | SLYLQMNSLRA | DFTLTISRLE | VTCVVVDVSHEDPEVKFNWYVDGVEVHNAKTKPREEQY | VVCLLNNFYPREAKVQWKV |
| | | | | EDTAVYYCARD | PEDFAVYYCQ | NSTYRVVSVLTVLHQDWLNGKEYKCKVSNKALPAPIEK | DNALQSGNSQESVTEQDSK |
| | | | | RGVGGTDYYYY | QYGSSPPYTF | TISKAKGQPREPQVYTLPPSREEMTKNQVSLTCLVKGF | DSTYSLSSTLTLSKADYEK |
| | | | | GLDVWGQGTTV | GQGTKVEIK | YPSDIAVEWESNGQPENNYKTTPPVLDSDGSFFLYSKL | HKVYACEVTHQGLSSPVTK |
| | | | | TVSS | | TVDKSRWQQGNVFSCSVMHEALHNHYTQKSLSLSPGK | SFNRGEC |
| | | | | 4417 | 4418 | 4419 | 4420 |
| 131 | VG9B387 | IgG1 | Lambda | EVQLVESGAEV | SYELTQDPAV | EVQLVESGAEVKKPGASVKVSCKASGFTFTGYYLQWVR | SYELTQDPAVSVALGQTVR |
| | | | | KKPGASVKVSC | SVALGQTVRI | QAPGQGLEWMGWINPNSGYTDYAQRFQDRVTMTRDTSI | ITCQGDSLRNYYAIWYQQK |
| | | | | KASGFTFTGYY | TCQGDSLRNY | NTAYMELSRLRSDDTAVYYCARLDDAFDVWGQGTMVTV | PGQAPVLVIFGKNNRPSGI |
| | | | | LQWVRQAPGQG | YAIWYQQKPG | SSASTKGPSVFPLAPSSKSTSGGTAALGCLVKDYFPEP | PDRFSGSSSGTTASLTITG |
| | | | | LEWMGWINPNS | QAPVLVIFGK | VTVSWNSGALTSGVHTFPAVLQSSGLYSLSSVVTVPSS | TQAEDEADYYCNSRDSSGN |
| | | | | GYTDYAQRFQD | NNRPSGIPDR | SLGTQTYICNVNHKPSNTKVDKKVEPKSCDKTHTCPPC | HWVFGGGTKLTVLGQPKAA |
| | | | | RVTMTRDTSIN | FSGSSSGTTA | PAPELLGGPSVFLFPPKPKDTLMISRTPEVTCVVVDVS | PSVTLFPPSSEELQANKAT |
| | | | | TAYMELSRLRS | SLTITGTQAE | HEDPEVKFNWYVDGVEVHNAKTKPREEQYNSTYRVVSV | LVCLISDFYPGAVTVAWKA |
| | | | | DDTAVYYCARL | DEADYYCNSR | LTVLHQDWLNGKEYKCKVSNKALPAPIEKTISKAKGQP | DSSPVKAGVETTTPSKQSN |
| | | | | DDAFDVWGQGT | DSSGNHWVFG | REPQVYTLPPSREEMTKNQVSLTCLVKGFYPSDIAVEW | NKYAASSYLSLTPEQWKSH |
| | | | | MVTVSS | GGTKLTVL | ESNGQPENNYKTTPPVLDSDGSFFLYSKLTVDKSRWQQ | RSYSCQVTHEGSTVEKTVA |
| | | | | | | GNVFSCSVMHEALHNHYTQKSLSLSPGK | PTECS |
| | | | | 4451 | 4452 | 4453 | 4454 |
| 132 | VG9B47 | IgG1 | Kappa | QVQLVQSGAEV | DIVMTQSPLS | QVQLVQSGAEVKKPGSSVKVSCKASGGSFSNYAISWMR | DIVMTQSPLSSPVDLGQPA |
| | | | | KKPGSSVKVSC | SPVDLGQPAS | QAPGQGLEWMGGIIPFFGTPDYAQKFQGRVTITADKST | SISCRSSQSLVHSDGNTYL |
| | | | | KASGGSFSNYA | ISCRSSQSLV | STAYMELSSLRSEDTAVYYCSTGGGYGDYDYYYGINVW | SWLQQRPGQPPRLLIYKIS |
| | | | | ISWMRQAPGQG | HSDGNTYLSW | GQGTMVTVSSASTKGPSVFPLAPSSKSTSGGTAALGCL | NRFSGVPDRFSGSGAGTDF |
| | | | | LEWMGGIIPFF | LQQRPGQPPR | VKDYFPEPVTVSWNSGALTSGVHTFPAVLQSSGLYSLS | TLKISRVEAEDVGVYYCMQ |
| | | | | GTPDYAQKFQG | LLIYKISNRF | SVVTVPSSSLGTQTYICNVNHKPSNTKVDKKVEPKSCD | AKEFPLTLGGGTKVDIKRT |
| | | | | RVTITADKSTS | SGVPDRFSGS | KTHTCPPCPAPELLGGPSVFLFPPKPKDTLMISRTPEV | VAAPSVFIFPPSDEQLKSG |
| | | | | TAYMELSSLRS | GAGTDFTLKI | TCVVVDVSHEDPEVKFNWYVDGVEVHNAKTKPREEQYN | TASVVCLLNNFYPREAKVQ |
| | | | | EDTAVYYCSTG | SRVEAEDVGV | STYRVVSVLTVLHQDWLNGKEYKCKVSNKALPAPIEKT | WKVDNALQSGNSQESVTEQ |
| | | | | GGYGDYDYYYG | YYCMQAKEFP | ISKAKGQPREPQVYTLPPSREEMTKNQVSLTCLVKGFY | DSKDSTYSLSSTLTLSKAD |
| | | | | INVWGQGTMVT | LTLGGGTKVD | PSDIAVEWESNGQPENNYKTTPPVLDSDGSFFLYSKLT | YEKHKVYACEVTHQGLSSP |
| | | | | VSS | IK | VDKSRWQQGNVFSCSVMHEALHNHYTQKSLSLSPGK | VTKSFNRGEC |
| | | | | 4485 | 4486 | 4487 | 4488 |
| 133 | VG9B379 | IgG1 | Lambda | EVQLVQSGPEV | SSELTQDPAV | EVQLVQSGPEVKKPGASVKVSCKASGYTFTGDYIHWVR | SSELTQDPAVSVALGQTVR |
| | | | | KKPGASVKVSC | SVALGQTVRI | QVPGQGLEWMGWINPNSGGTNYAQKFQGRVTMTRDTSI | ITCQGDSLRSYYARWYQQK |
| | | | | KASGYTFTGDY | TCQGDSLRSY | STAFMELSRLRSDDTAVYYCAREGGVAPAAPDAFDIWG | PGQAPVVVIYGKNNRPSGI |
| | | | | IHWVRQVPGQG | YARWYQQKPG | QGTMVTVSSASTKGPSVFPLAPSSKSTSGGTAALGCLV | PDRFSGSSSGNTASLTITG |
| | | | | LEWMGWINPNS | QAPVVVIYGK | KDYFPEPVTVSWNSGALTSGVHTFPAVLQSSGLYSLSS | AQAEDEADYYCNSRDSSGN |
| | | | | GGTNYAQKFQG | NNRPSGIPDR | VVTVPSSSLGTQTYICNVNHKPSNTKVDKKVEPKSCDK | HRVFGGGTKLTVLGQPKAA |
| | | | | RVTMTRDTSIS | FSGSSSGNTA | THTCPPCPAPELLGGPSVFLFPPKPKDTLMISRTPEVT | PSVTLFPPSSEELQANKAT |
| | | | | TAFMELSRLRS | SLTITGAQAE | CVVVDVSHEDPEVKFNWYVDGVEVHNAKTKPREEQYNS | LVCLISDFYPGAVTVAWKA |
| | | | | DDTAVYYCARE | DEADYYCNSR | TYRVVSVLTVLHQDWLNGKEYKCKVSNKALPAPIEKTI | DSSPVKAGVETTTPSKQSN |
| | | | | GGVAPAAPDAF | DSSGNHRVFG | SKAKGQPREPQVYTLPPSREEMTKNQVSLTCLVKGFYP | NKYAASSYLSLTPEQWKSH |
| | | | | DIWGQGTMVTV | GGTKLTVL | SDIAVEWESNGQPENNYKTTPPVLDSDGSFFLYSKLTV | RSYSCQVTHEGSTVEKTVA |
| | | | | SS | | DKSRWQQGNVFSCSVMHEALHNHYTQKSLSLSPGK | PTECS |
| | | | | 4519 | 4520 | 4521 | 4522 |
| 134 | VG9B447 | IgG1 | Lambda | QVQLQESGPGL | SYELTQPASV | QVQLQESGPGLVKPSETLSLTCTVSGGSISNYYWSWIR | SYELTQPASVSGSPGQSIT |
| | | | | VKPSETLSLTC | SGSPGQSITI | QPAGKGLGWIGRIYSSGITNYNPSLKSRVTMSVDTSKN | ISCTGTSSDVGGYNYVSWY |
| | | | | TVSGGSISNYY | SCTGTSSDVG | QLSLKLSSVTAADTAVYYCAGIVGVKGAFAIWGQGTMV | QQHPGKAPKLMIYEVSNRP |
| | | | | WSWIRQPAGKG | GYNYVSWYQQ | TVSSASTKGPSVFPLAPSSKSTSGGTAALGCLVKDYFP | SGVSNRFSGSKSGNTASLT |
| | | | | LGWIGRIYSSG | HPGKAPKLMI | EPVTVSWNSGALTSGVHTFPAVLQSSGLYSLSSVVTVP | ISGLQAEDEADYYCSSYTS |
| | | | | ITNYNPSLKSR | YEVSNRPSGV | SSSLGTQTYICNVNHKPSNTKVDKKVEPKSCDKTHTCP | TSVVFGGGTKLTVLGQPKA |
| | | | | VTMSVDTSKNQ | SNRFSGSKSG | PCPAPELLGGPSVFLFPPKPKDTLMISRTPEVTCVVVD | APSVTLFPPSSEELQANKA |
| | | | | LSLKLSSVTAA | NTASLTISGL | VSHEDPEVKFNWYVDGVEVHNAKTKPREEQYNSTYRVV | TLVCLISDFYPGAVTVAWK |
| | | | | DTAVYYCAGIV | QAEDEADYYC | SVLTVLHQDWLNGKEYKCKVSNKALPAPIEKTISKAKG | ADSSPVKAGVETTTPSKQS |
| | | | | GVKGAFAIWGQ | SSYTSTSVVF | QPREPQVYTLPPSREEMTKNQVSLTCLVKGFYPSDIAV | NNKYAASSYLSLTPEQWKS |
| | | | | GTMVTVSS | GGGTKLTVL | EWESNGQPENNYKTTPPVLDSDGSFFLYSKLTVDKSRW | HRSYSCQVTHEGSTVEKTV |
| | | | | | | QQGNVFSCSVMHEALHNHYTQKSLSLSPGK | APTECS |
| | | | | 4553 | 4554 | 4555 | 4556 |
| 135 | VG9B392 | IgG1 | Lambda | QVQLVQSGAEV | SSELTQDPAV | QVQLVQSGAEVKRPGASAKVSCKTSGYTFTGYYIHWVR | SSELTQDPAVSVALGQTVR |
| | | | | KRPGASAKVSC | SVALGQTVRI | QAPGQGLEWMGWINPNSGYTNSAQKFQGRVSMTRDTSI | ITCQGDSLRSYYARWYQQK |
| | | | | KTSGYTFTGYY | TCQGDSLRSY | STVYMELSRLRSDDTAVYYCAREDDAFDIWGQGTLVTV | PGQAPVLVIYGKNNRPSGI |
| | | | | IHWVRQAPGQG | YARWYQQKPG | SSASTKGPSVFPLAPSSKSTSGGTAALGCLVKDYFPEP | PDRFSGSSSGDTASLTITG |
| | | | | LEWMGWINPNS | QAPVLVIYGK | VTVSWNSGALTSGVHTFPAVLQSSGLYSLSSVVTVPSS | ALAEDEADYYCNSRDNSGN |
| | | | | GYTNSAQKFQG | NNRPSGIPDR | SLGTQTYICNVNHKPSNTKVDKKVEPKSCDKTHTCPPC | HVVFGGGTKVTVLGQPKAA |
| | | | | RVSMTRDTSIS | FSGSSSGDTA | PAPELLGGPSVFLFPPKPKDTLMISRTPEVTCVVVDVS | PSVTLFPPSSEELQANKAT |
| | | | | TVYMELSRLRS | SLTITGALAE | HEDPEVKFNWYVDGVEVHNAKTKPREEQYNSTYRVVSV | LVCLISDFYPGAVTVAWKA |
| | | | | DDTAVYYCARE | DEADYYCNSR | LTVLHQDWLNGKEYKCKVSNKALPAPIEKTISKAKGQP | DSSPVKAGVETTTPSKQSN |
| | | | | DDAFDIWGQGT | DNSGNHVVFG | REPQVYTLPPSREEMTKNQVSLTCLVKGFYPSDIAVEW | NKYAASSYLSLTPEQWKSH |
| | | | | LVTVSS | GGTKVTVL | ESNGQPENNYKTTPPVLDSDGSFFLYSKLTVDKSRWQQ | RSYSCQVTHEGSTVEKTVA |
| | | | | | | GNVFSCSVMHEALHNHYTQKSLSLSPGK | PTECS |
| | | | | 4587 | 4588 | 4589 | 4590 |
| 136 | VG9B96 | IgG1 | Kappa | EVQLVQSGAEV | DIVMTQSPLS | EVQLVQSGAEVKKPGSSVKVSCKASGGTFSNYAISWVR | DIVMTQSPLSSHVTLGQPA |
| | | | | KKPGSSVKVSC | SHVTLGQPAS | LAPGQGLEWMGGIIPIFSAGTYAQRFQGRVTITADKST | SISCRSSQSLVHSDGNTYL |
| | | | | KASGGTFSNYA | ISCRSSQSLV | STAYMELNSLRSEDTAVYYCSSNSGTYYDYYYGMDVWG | SWLQQRPGQPPRLLIYKIF |
| | | | | ISWVRLAPGQG | HSDGNTYLSW | QGTMVTVSSASTKGPSVFPLAPSSKSTSGGTAALGCLV | NRLSGVPDRFSGSGAGTDF |
| | | | | LEWMGGIIPIF | LQQRPGQPPR | KDYFPEPVTVSWNSGALTSGVHTFPAVLQSSGLYSLSS | TLKISRVEAEDVGVYYCMQ |
| | | | | SAGTYAQRFQG | LLIYKIFNRL | VVTVPSSSLGTQTYICNVNHKPSNTKVDKKVEPKSCDK | AKQFPLTFGGGTKVEIKRT |
| | | | | RVTITADKSTS | SGVPDRFSGS | THTCPPCPAPELLGGPSVFLFPPKPKDTLMISRTPEVT | VAAPSVFIFPPSDEQLKSG |
| | | | | TAYMELNSLRS | GAGTDFTLKI | CVVVDVSHEDPEVKFNWYVDGVEVHNAKTKPREEQYNS | TASVVCLLNNFYPREAKVQ |
| | | | | EDTAVYYCSSN | SRVEAEDVGV | TYRVVSVLTVLHQDWLNGKEYKCKVSNKALPAPIEKTI | WKVDNALQSGNSQESVTEQ |
| | | | | SGTYYDYYYGM | YYCMQAKQFP | SKAKGQPREPQVYTLPPSREEMTKNQVSLTCLVKGFYP | DSKDSTYSLSSTLTLSKAD |
| | | | | DVWGQGTMVTV | LTFGGGTKVE | SDIAVEWESNGQPENNYKTTPPVLDSDGSFFLYSKLTV | YEKHKVYACEVTHQGLSSP |
| | | | | SS | IK | DKSRWQQGNVFSCSVMHEALHNHYTQKSLSLSPGK | VTKSFNRGEC |
| | | | | 4621 | 4622 | 4623 | 4624 |
| 137 | VG9B77 | IgG1 | Kappa | EVQLVESGAEV | EIVMTQSPLF | EVQLVESGAEVMKPGSSVKVSCKASGGTFSSYAISWVR | EIVMTQSPLFSPVTLGQPA |
| | | | | MKPGSSVKVSC | SPVTLGQPAS | QAPEQGLEWMGGIIPFFGTADYAQKFQGRVTITADKST | SISCRSSQSLVHSDGNTYL |
| | | | | KASGGTFSSYA | ISCRSSQSLV | NTAYMELTSLRSEDTAVYYCATATVTTDYYYGMDVWGQ | SWLQQRPGQPPRLLIYEIS |
| | | | | ISWVRQAPEQG | HSDGNTYLSW | GTMVTVSSASTKGPSVFPLAPSSKSTSGGTAALGCLVK | NRFTGVPDRFSGSGAGTDF |
| | | | | LEWMGGIIPFF | LQQRPGQPPR | DYFPEPVTVSWNSGALTSGVHTFPAVLQSSGLYSLSSV | TLKISRVEAEDVGVYYCMQ |
| | | | | GTADYAQKFQG | LLIYEISNRF | VTVPSSSLGTQTYICNVNHKPSNTKVDKKVEPKSCDKT | ARQFPLTFGGGTKVEIKRT |
| | | | | RVTITADKSTN | TGVPDRFSGS | HTCPPCPAPELLGGPSVFLFPPKPKDTLMISRTPEVTC | VAAPSVFIFPPSDEQLKSG |
| | | | | TAYMELTSLRS | GAGTDFTLKI | VVVDVSHEDPEVKFNWYVDGVEVHNAKTKPREEQYNST | TASVVCLLNNFYPREAKVQ |
| | | | | EDTAVYYCATA | SRVEAEDVGV | YRVVSVLTVLHQDWLNGKEYKCKVSNKALPAPIEKTIS | WKVDNALQSGNSQESVTEQ |
| | | | | TVTTDYYYGMD | YYCMQARQFP | KAKGQPREPQVYTLPPSREEMTKNQVSLTCLVKGFYPS | DSKDSTYSLSSTLTLSKAD |
| | | | | VWGQGTMVTVS | LTFGGGTKVE | DIAVEWESNGQPENNYKTTPPVLDSDGSFFLYSKLTVD | YEKHKVYACEVTHQGLSSP |
| | | | | S | IK | KSRWQQGNVFSCSVMHEALHNHYTQKSLSLSPGK | VTKSFNRGEC |
| | | | | 4655 | 4656 | 4657 | 4658 |

TABLE 3
Kabat CDR Amino Acid Sequences

| | Protein | HC Kabat | | | | LC Kabat | LC Kabat |
|---|---|---|---|---|---|---|---|
| # | Name | CDR1 | HC Kabat CDR2 | HC Kabat CDR3 | LC Kabat CDR1 | CDR2 | CDR3 |
| 1 | VG9B54 | SYSLT | SISSSSSYIFYADSVKG | DGELGPFDY | RASQSVSSSYLA | GASSRAT | QQYGRSPLT |
| | | 1 | 2 | 3 | 4 | 5 | 6 |
| 2 | VG9B121 | DYAMD | SISSTSNYIFYADSVKG | SYNWNYGGAFDI | RASQSVSSSYFA | GASSRAT | QQYGRSPLT |
| | | 35 | 36 | 37 | 38 | 39 | 40 |
| 3 | VG9B429 | NYDMN | SISSSSSYIYYADSVKG | DVGVTTDYYYYGMDV | RASQGINSWLA | AASSLQS | QQANSFPWT |
| | | 69 | 70 | 71 | 72 | 73 | 74 |
| 4 | VG9B370 | SYDMN | SISSSSSYIYYADSVKG | DRGVGGTDYYYYGLDV | RASQSVSSSYLA | GASSRAT | QQYGSSPPYT |
| | | 103 | 104 | 105 | 106 | 107 | 108 |
| 5 | VG9B80 | TYSMN | SISSSSSYIFYADSVKG | DGELGVFDY | RASQSISSSYLA | GPSGRAT | QQFGRSPLT |
| | | 137 | 138 | 139 | 140 | 141 | 142 |
| 6 | VG9B414 | NYDMN | SISSSSSYIYYADSVKG | DVGVTTDYYYYGMDV | RASQGINSWLA | AASSLQS | QQANSFPWT |
| | | 171 | 172 | 173 | 174 | 175 | 176 |
| 7 | VG9B195 | PYTMN | SISSSSSYMYYADSVKG | DGDLVGPTYYFDY | SGDKLGDKYAC | QHNKRPS | QAWDSTTVV |
| | | 205 | 206 | 207 | 208 | 209 | 210 |
| 8 | VG9B140 | SYDMN | SISTSSGYIYYADSVKG | DRGIAVAGDYYYGMDV | RASQSVSSSYLA | GASSRAT | QQYGSSPPYT |
| | | 239 | 240 | 241 | 242 | 243 | 244 |
| 9 | VG9B426 | NYDMN | SISSSSSYIYYADSVKG | DVGVTTDYYYYGMDV | RASQSVASSYLA | GASSRAT | QQYGSSPPYT |
| | | 273 | 274 | 275 | 276 | 277 | 278 |
| 10 | VG9B46 | INSMN | SISSTSDYIFNADSVKG | DDVFGAFDI | RTSQSVSRSYLG | GSSSRAT | QQYSRSPLT |
| | | 307 | 308 | 309 | 310 | 311 | 312 |
| 11 | VG9B416 | NYDMN | SISSSSSYIYYADSVKG | DVGVTTDYYYYGMDV | RASQSVASSYLA | GASSRAT | QQYGSSPPYT |
| | | 341 | 342 | 343 | 344 | 345 | 346 |
| 12 | VG9B69 | SYDIN | SITSSSYYIYYADSVKG | DLGVRGVDYYYYGLDV | RASQSVSSSYLA | GASSRAT | QQYGSSPPYT |
| | | 375 | 376 | 377 | 378 | 379 | 380 |
| 13 | VG9B415 | NYDMN | SISSSSSYIYYADSVKG | DVGVTTDYYYYGMDV | RASQSVASSYLA | GASSRAT | QQYGSSPPYT |
| | | 409 | 410 | 411 | 412 | 413 | 414 |
| 14 | VG9B104 | VYSMN | SIGSSSSYIFYADSVKG | DHDYGGLDY | RASQSVSSSYLA | GPSNRAT | QQYGRSPLT |
| | | 443 | 444 | 445 | 446 | 447 | 448 |
| 15 | VG9B198 | SIYWS | RIYTTDITNYNPSLKS | NGYSYGGFNY | TGSSSNIGAGYDVH | GDSYRPS | QSYDSSLSVVV |
| | | 477 | 478 | 479 | 480 | 481 | 482 |
| 16 | VG9B463 | NYDMN | SISSSSSYIYYADSVKG | DVGVTTDYYYYGMDV | SGSSSNIGSNTVN | TNTQRPS | AAWDDSLNAWV |
| | | 511 | 512 | 513 | 514 | 515 | 516 |
| 17 | VG9B469 | HYDMN | SISSSSSYIFYADSVKG | DRGVGDTSDYYSFGLDV | RASQNVSSTYLA | GACSRAT | QQYGSSPPYT |
| | | 545 | 546 | 547 | 548 | 549 | 550 |
| 18 | VG9B428 | NYGMN | YISSGSSYKYYADSMKG | DPVVTEYYYYGMDV | RASQGINSWLA | AASSLQS | QQANSFPWT |
| | | 579 | 580 | 581 | 582 | 583 | 584 |
| 19 | VG9B430 | SYDMN | SISSSSSYIYYADSVKG | DVGVTTDYYYYGMDV | RASQSVASSYLA | GASSRAT | QQYGSSPPYT |
| | | 613 | 614 | 615 | 616 | 617 | 618 |
| 20 | VG9B423 | NYDMN | SISSSSSYIYYADSVKG | DVGVTTDYYYYGMDV | RASQSVASSYLA | GASSRAT | QQYGSSPPYT |
| | | 647 | 648 | 649 | 650 | 651 | 652 |
| 21 | VG9B98 | SYSMY | SIGSSSTYIFYADSVKG | DGELGPFEY | RASQSVSTSYLA | GTSSRAT | LQYGRSPLT |
| | | 681 | 682 | 683 | 684 | 685 | 686 |
| 22 | VG9B73 | NYDMN | SISSSSSYIYYADSVKG | EIGVTGTTYYQDYGMDV | RASQSFSSNYLA | GASSRAT | QQYGSSPPFT |
| | | 715 | 716 | 717 | 718 | 719 | 720 |
| 23 | VG9B133 | NYDMN | SISSSSSYIYYADSVKG | DLGITGTTMDYYYGMDV | RASQSVSSSYLA | GASSRAT | QQYGSSPPYT |
| | | 749 | 750 | 751 | 752 | 753 | 754 |
| 24 | VG9B368 | SYDMN | SISSSSSYIYYADSVKG | DRGIGGDYYSYAMDV | RASQSVSSSYLA | GASSRAT | QQYGSSPPYT |
| | | 783 | 784 | 785 | 786 | 787 | 788 |
| 25 | VG9B424 | NYDMN | SISSSSSYIYYADSVKG | DVGVTTDYYYYGMDV | RASQSVASSYLA | GASSRAT | QQYGSSPPYT |
| | | 817 | 818 | 819 | 820 | 821 | 822 |
| 26 | VG9B427 | NYDMN | SISSSSSYIYYADSVKG | DVGVTTDYYYYGMDV | RASQSVASSYLA | GASSRAT | QQYGSSPPYT |
| | | 851 | 852 | 853 | 854 | 855 | 856 |
| 27 | VG9B417 | NYGMN | YISSGSSYKYYADSMKG | DPVVTEYYYYGMDV | RASQGINSWLA | AASSLQS | QQANSFPWT |
| | | 885 | 886 | 887 | 888 | 889 | 890 |
| 28 | VG9B58 | SSYWT | YMFYLGSTNYNPSLKS | ERPVLDAFDI | RASQDITNYLA | AASSLQG | LQHDTYPYT |
| | | 919 | 920 | 921 | 922 | 923 | 924 |
| 29 | VG9B419 | NYDMN | SISSSSSYIYYADSVKG | DVGVTTDYYYYGMDV | RASQSVASSYLA | GASSRAT | QQYGSSPPYT |
| | | 953 | 954 | 955 | 956 | 957 | 958 |
| 30 | VG9B425 | NYDMN | SISSSSSYIYYADSVKG | DVGVTTDYYYYGMDV | RASQSVASSYLA | GASSRAT | QQYGSSPPYT |
| | | 987 | 988 | 989 | 990 | 991 | 992 |
| 31 | VG9B143 | TYGMN | YISTSSYTIYYSDSVKG | EGDWWYFDL | TGTSSDVGGYNYVS | EVSNRPS | SSYTSSSTLV |
| | | 1021 | 1022 | 1023 | 1024 | 1025 | 1026 |
| 32 | VG9B418 | NYDMN | SISSSSSYIYYADSVKG | DVGVTTDYYYYGMDV | RASQSVASSYLA | GASSRAT | QQYGSSPPYT |
| | | 1055 | 1056 | 1057 | 1058 | 1059 | 1060 |
| 33 | VG9B472 | HYDMN | SISSSSSYIFYADSVKG | DRGVGDTSDYYSFGLDV | RASQGIATYLA | AASTLQS | QKYNSAPPWT |
| | | 1089 | 1090 | 1091 | 1092 | 1093 | 1094 |
| 34 | VG9B421 | NYGMN | YISSGSSYKYYADSMKG | DPVVTEYYYYGMDV | RASQGINSWLA | AASSLQS | QQANSFPWT |
| | | 1123 | 1124 | 1125 | 1126 | 1127 | 1128 |
| 35 | VG9B88 | SYTMN | SISTSSSYIDYADSVKG | DGDMVAPIKGSFDY | RSSQSLLNSDDGNTYLD | TLSYRAS | MQRIEFPIT |
| | | 1157 | 1158 | 1159 | 1160 | 1161 | 1162 |
| 36 | VG9B384 | SSGMCVS | LIDWFDDKYYSTSLKT | IRGTGAYYYGLDV | SGNELGDKYAS | QDNKRPS | QAWDSSKVV |
| | | 1191 | 1192 | 1193 | 1194 | 1195 | 1196 |
| 37 | VG9B413 | NYDMN | SISSSSSYIYYADSVKG | DVGVTTDYYYYGMDV | RASQSVASSYLA | GASSRAT | QQYGSSPPYT |
| | | 1225 | 1226 | 1227 | 1228 | 1229 | 1230 |
| 38 | VG9B36 | SYGMY | SISTGSSYIYYADSVKG | DKGLAVTGYIMDV | QASQDISNYLN | DASNLET | QQYDNLPMYT |
| | | 1259 | 1260 | 1261 | 1262 | 1263 | 1264 |
| 39 | VG9B403 | NYDMN | SISSSSSYIYYADSVKG | DVGVTTDYYYYGMDV | RASQSVASSYLA | GASSRAT | QQYGSSPPYT |
| | | 1293 | 1294 | 1295 | 1296 | 1297 | 1298 |
| 40 | VG9B191 | RYSMN | SISSSSSYIYYADSVKG | DGPTVNWDYYFDL | SGSSSNIGNNYVS | DNNKRPS | GTWDSSLSTSVV |
| | | 1327 | 1328 | 1329 | 1330 | 1331 | 1332 |
| 41 | VG9B44 | SNSAAWN | RTYYRSKWYNDYPISVKS | ESGSYYTDGFDI | RASQGISSYLA | AASTLQS | QEFNSYPYT |
| | | 1361 | 1362 | 1363 | 1364 | 1365 | 1366 |
| 42 | VG9B67 | SYTMN | SISSSSSYIDYAESVKG | DGDILATIRGSFDY | RSSQSLLDSDAGNTYLD | TLSYRAS | MQRIEFPIT |
| | | 1395 | 1396 | 1397 | 1398 | 1399 | 1400 |
| 43 | VG9B402 | NYGMN | YISSGSSYKYYADSMKG | DPVVTEYYYYGMDV | RASQGINSWLA | AASSLQS | QQANSFPWT |
| | | 1429 | 1430 | 1431 | 1432 | 1433 | 1434 |
| 44 | VG9B127 | AYTMN | SISSSSSYIDFAESVKG | DGDIVSTIRGSFDY | RSSQSLLDSDDGNTYLD | TLSYRAS | MQRIEFPIT |
| | | 1463 | 1464 | 1465 | 1466 | 1467 | 1468 |
| 45 | VG9B137 | SYSMD | SIGSSSSYIFYADSVKG | SYSWNYGGAFDI | RASQSVSSSYLA | GASSRAT | QQYGSSPPYT |
| | | 1497 | 1498 | 1499 | 1500 | 1501 | 1502 |
| 46 | VG9B33 | SYNMN | SISTSSSYIYYADSVKG | DTSVTKYPDTFDI | RASQDINNYLA | GASTLQS | QKYNSAPFT |
| | | 1531 | 1532 | 1533 | 1534 | 1535 | 1536 |
| 47 | VG9B162 | GDYMH | WINPNSGYTNYAQKFQG | EGDAFDV | TGTSSDVGGYNYVS | EVSKRPS | NSYAGSNNFEV |
| | | 1565 | 1566 | 1567 | 1568 | 1569 | 1570 |
| 48 | VG9B152 | SYSMI | SISSSSDYIYNADSVKG | DWELLGFDC | SGDKLGDKYAC | QHNKRPS | QAWDSTTVV |
| | | 1599 | 1600 | 1601 | 1602 | 1603 | 1604 |
| 49 | VG9B64 | ISGVSVG | LIYWNDDKRYSPSLQS | SGQWLEGDAFDI | RSSESLVHSDGNTYLS | KISNRFS | MQATQFPLT |
| | | 1633 | 1634 | 1635 | 1636 | 1637 | 1638 |
| 50 | VG9B21 | NYDMN | SISSSSHYIYYADSLKG | DRGVTTDYYYYALDV | RASQGIYNYLA | AASTLQS | QKYNRAPFT |
| | | 1667 | 1668 | 1669 | 1670 | 1671 | 1672 |
| 51 | VG9B128 | TYGMH | VIWYNGSNKYYADSVKG | GGFGESFDS | RASQSVIDYLA | DASNRAT | QQRSNWPLT |
| | | 1701 | 1702 | 1703 | 1704 | 1705 | 1706 |
| 52 | VG9B66 | TYTMN | SISSSSFYMDYADSVKG | DGDIVATIRGSFDY | RASQDITNFLA | TASTLQS | QKYNSAPFT |
| | | 1735 | 1736 | 1737 | 1738 | 1739 | 1740 |
| 53 | VG9B32 | RYAMN | FISGTGYTVYYADSVKG | DQEPGFDY | RSGQSLVHSDGNTYLS | KISSNRFS | MQATHFPFT |
| | | 1769 | 1770 | 1771 | 1772 | 1773 | 1774 |
| 54 | VG9B57 | SHDMN | SISSSSSYIFYADSVKG | DLGVGVRDYYYYGMDV | RASQDCNYLA | AASSLQG | LQHDTYPYT |
| | | 1803 | 1804 | 1805 | 1806 | 1807 | 1808 |
| 55 | VG9B135 | TYGVGVG | LIYWNDDKRYYPSLNN | DYDFWSGYFDY | RASQSVSSSYLA | GASSRAT | QQYGSSPLT |
| | | 1837 | 1838 | 1839 | 1840 | 1841 | 1842 |
| 56 | VG9B60 | GYYMH | RINPNSGVTHYAQKFQG | GGSLVRGVISGLDY | RASQSFSGSYLA | GASSRAT | QQYGSSPPYT |
| | | 1871 | 1872 | 1873 | 1874 | 1875 | 1876 |
| 57 | VG9B409 | SYYMH | IINPSGGSTSYAQKFQG | GSYGWYFDL | RASQSVTSSYLA | GASSRAT | QQYGSSPPYT |
| | | 1905 | 1906 | 1907 | 1908 | 1909 | 1910 |
| 58 | VG9B411 | NYDMN | SISSSSSYIYYADSVKG | DVGVTTDYYYYGMDV | RASQGINSWLA | AASTLQS | QQYNNWPRT |
| | | 1939 | 1940 | 1941 | 1942 | 1943 | 1944 |
| 59 | VG9B129 | SYVIS | GILPILSTANYAQKFQG | AHDYYYGMDV | RASQSVSNNYLA | GASSRAT | QQYGSSPPYT |
| | | 1973 | 1974 | 1975 | 1976 | 1977 | 1978 |
| 60 | VG9B396 | GYYWN | RIFTTGNTNYNPSLKS | EKWDSSSSALYFDF | TGSSSNIGADYDVK | GNTDRPS | QSYDSRLTGYVV |
| | | 2007 | 2008 | 2009 | 2010 | 2011 | 2012 |
| 61 | VG9B470 | TSGVGVG | LILWNDHTIYSPSLKS | DKWELRDAFDI | RASQGISRYLA | VASTLQS | QQLISYPYT |
| | | 2041 | 2042 | 2043 | 2044 | 2045 | 2046 |
| 62 | VG9B111 | TYSVN | SISSDSSYIFYADSMKG | DSVTGPFDY | QASQDISHYLN | DSYILET | QQYDNLPYT |
| | | 2075 | 2076 | 2077 | 2078 | 2079 | 2080 |
| 63 | VG9B169 | NYFWN | YIFYSGSTSYNPSLKS | VGRWELRTAFDI | SGSSSNIGSNTVN | SNNQRPS | AAWDDSLNGPGV |
| | | 2109 | 2110 | 2111 | 2112 | 2113 | 2114 |
| 64 | VG9B639 | TYRMN | SISSSSIYIHSADSVKG | ERVYTVSFDY | QASQDISHYLN | DSYILET | QQYDNLPYT |
| | | 2143 | 2144 | 2145 | 2146 | 2147 | 2148 |
| 65 | VG9B201 | THGVGVG | LIYWNADKHYSPSLKS | EGDWGHYFDF | SGSSSNIGNNDVS | DNNRRPS | ETWDSSLSAIWV |
| | | 2177 | 2178 | 2179 | 2180 | 2181 | 2182 |
| 66 | VG9B161 | SYFWS | YIFYSGSTNYNPSLKS | VGRWELRGAFDI | SGSSSNIGSNTVN | SNNQRPS | AAWDDSLNSPGV |
| | | 2211 | 2212 | 2213 | 2214 | 2215 | 2216 |
| 67 | VG9B383 | SYYWN | RIYTIGNTNYNPSLKS | EGYYDSSGSFFPGAFGI | TGTSNDVGSYNLVS | AGSKRPS | CSFAGATNVV |
| | | 2245 | 2246 | 2247 | 2248 | 2249 | 2250 |
| 68 | VG9B382 | GYYWN | RIFTTGNTNYNPSLKS | ERWDSSSSALYFDY | TGSSSNIGADYDIK | GNSNRPS | QSYDSSMSGYVV |
| | | 2279 | 2280 | 2281 | 2282 | 2283 | 2284 |
| 69 | VG9B156 | GYFWN | YIFYSGSTNYNPSLKS | LGKWELRTAFDI | SGSTSNIGSDTVN | SNNQRPS | AAWDDSLNGPV |
| | | 2313 | 2314 | 2315 | 2316 | 2317 | 2318 |
| 70 | VG9B205 | GYFWN | YIFYSGSTNYNPSLKS | LGKWELRTAFDI | SGDKLGDKYAC | QDSKRPS | QAWDSSTVV |
| | | 2347 | 2348 | 2349 | 2350 | 2351 | 2352 |
| 71 | VG9B86 | SSYWS | RFYSSGSTSYNPSLKS | YSGSYWYFDL | RASQSVSSYLA | DTSNRAT | QQRSDWLLT |
| | | 2381 | 2382 | 2383 | 2384 | 2385 | 2386 |
| 72 | VG9B154 | YYFWN | YIYYSGSTNYNPSLKS | EGKWELRTTFDI | SGSSSNIGSNTVN | SNNQRPS | AAWDDSLNGPV |
| | | 2415 | 2416 | 2417 | 2418 | 2419 | 2420 |
| 73 | VG9B159 | SYYWS | RFYTGGRNNYNPSFKS | DMEYYYDRSGYSYWYFDL | GGNNIGSKSVH | DDSDRPS | QVWDSSSDHVV |
| | | 2449 | 2450 | 2451 | 2452 | 2453 | 2454 |
| 74 | VG9B465 | SYYWN | RIYTIGNTNYNPSLKS | EGYYESDGSFFPGAFNI | TGTSNDVGSYNLVS | AGSKRPS | CSYAGTTNVV |
| | | 2483 | 2484 | 2485 | 2486 | 2487 | 2488 |
| 75 | VG9B194 | GDYMH | WINPNSGYTNNAEKFQG | ELDALDV | TGTSSDVGGYNYVS | EVSKRPS | NSYAGSNNFEV |
| | | 2517 | 2518 | 2519 | 2520 | 2521 | 2522 |
| 76 | VG9B182 | NHFWS | FVFYNGNTNYNPSLKS | VGRWVLRTAFDI | SGSSSNIGSNTVN | SNNQRPS | AAWDDSLNGPGV |
| | | 2551 | 2552 | 2553 | 2554 | 2555 | 2556 |
| 77 | VG9B173 | NYFWS | YIYYSGSTNYNPSLKS | EGKWELRSAFDI | SGSSSNIESNTVN | SNNQRPS | TAWDDSLNGPV |
| | | 2585 | 2586 | 2587 | 2588 | 2589 | 2590 |
| 78 | VG9B87 | SEYIH | IINPSGGSTSYAQRFQG | ERGYSYGSFDY | RASQGISSYLA | AASTLQS | QQFNSYSLT |
| | | 2619 | 2620 | 2621 | 2622 | 2623 | 2624 |
| 79 | VG9B208 | GYYWS | YIFYSGSINYNPSLKN | VGKWELRSSFDI | SGSTSNIGNNDVS | DNNKRPS | GTWDSSLSVWV |
| | | 2653 | 2654 | 2655 | 2656 | 2657 | 2658 |
| 80 | VG9B372 | NGGFYWS | YINYSGSTYYNPSLES | DRNYEWNFDL | RASQSVSSYLA | DASNRAT | QQRSNWPLT |
| | | 2687 | 2688 | 2689 | 2690 | 2691 | 2692 |
| 81 | VG9B186 | NIRMSVS | HIFSNDEKSYNSSLKS | MRLPYGMDV | SGSTSNIGSNTVN | SNNQRPS | AAWDDSLNGPV |
| | | 2721 | 2722 | 2723 | 2724 | 2725 | 2726 |
| 82 | VG9B177 | NYFWS | YIFYSGSTNYNPSLKS | VGKWELRTAFDI | TGTSSDVADYNYVS | EVSNRPS | CSYTSSFTVV |
| | | 2755 | 2756 | 2757 | 2758 | 2759 | 2760 |
| 83 | VG9B114 | RYDMH | AIGSAGDTYYPGSVKG | GKWELRDAFDI | RASQGIHSYLA | VASTLQS | QQLNSYPYT |
| | | 2789 | 2790 | 2791 | 2792 | 2793 | 2794 |
| 84 | VG9B147 | GYFWN | YIFYSGSTNYNPSLKS | EGKWELRSTFDI | QGDSLRSYYAT | GENNRPS | NSRDTGDHHLV |
| | | 2823 | 2824 | 2825 | 2826 | 2827 | 2828 |
| 85 | VG9B65 | IYAIN | GIIPFFGTANYAQKFQD | GGDSGYDWGFDY | RSSQSLVHSDGNTYLS | QISNRFS | MQATQFPLT |
| | | 2857 | 2858 | 2859 | 2860 | 2861 | 2862 |
| 86 | VG9B81 | TYYWS | RIYTSDNTNYNPSLKS | YNWNYWYFDL | RASQGISSYLA | TASTLQS | QHLNSYPYT |
| | | 2891 | 2892 | 2893 | 2894 | 2895 | 2896 |
| 87 | VG9B203 | FTYYWN | RIYTSGSTNYNPSLKS | SGGNFYWYFDL | SGSSSNIGNNYVS | DNNKRPS | GTWDSSLSAGV |
| | | 2925 | 2926 | 2927 | 2928 | 2929 | 2930 |
| 88 | VG9B380 | NYYWS | RIYPSGITSYDPSLKS | IMGTKGAFDI | TGTSSDVGGYNYVS | EVSNRPS | SSYTSTSVV |
| | | 2959 | 2960 | 2961 | 2962 | 2963 | 2964 |
| 89 | VG9B103 | SYAIS | GIIPIFGTATYAQKFQD | GVGWGTDYYYGLDV | RSSQGLVHSDGNTYLS | KISNRFS | MQATHHPLT |
| | | 2993 | 2994 | 2995 | 2996 | 2997 | 2998 |
| 90 | VG9B462 | NYYWS | RIYSSGITNYNPSLKS | IVGVKGAFAI | TGTSSDVGGYNYVS | EVSNRPS | SSYTSTSVV |
| | | 3027 | 3028 | 3029 | 3030 | 3031 | 3032 |
| 91 | VG9B461 | NYYWS | RIYSSGITNYNPSLKS | IVGVKGAFAI | TGTSSDVGGYNYVS | EVSNRPS | SSYTSTSVV |
| | | 3061 | 3062 | 3063 | 3064 | 3065 | 3066 |
| 92 | VG9B106 | GYYLH | WINPSSGDTDYAQTFQG | ELGIGVFDY | KSSQSVLYSSNNKNYLG | WASTRES | QQYYSIPYT |
| | | 3095 | 3096 | 3097 | 3098 | 3099 | 3100 |
| 93 | VG9B115 | TYGVGVG | LIYWNDDKRYNPSLKS | ESDWSYYFDY | RASQSVSSYLA | DASNRAT | QQRSSWPWT |
| | | 3129 | 3130 | 3131 | 3132 | 3133 | 3134 |
| 94 | VG9B27 | SYVIS | GILPILSTANYAQKFQG | AHDYYYGMDV | RSSQSLVHSDGNTYLS | KISNRFS | MQATHHPLT |
| | | 3163 | 3164 | 3165 | 3166 | 3167 | 3168 |
| 95 | VG9B458 | NYYWS | RIYSSGITNYNPSLKS | IVGVKGAFAI | TGTSSDVGGYNYVS | EVSNRPS | SSYTSTSVV |
| | | 3197 | 3198 | 3199 | 3200 | 3201 | 3202 |
| 96 | VG9B131 | SYDMN | YISSSSTAKYYADSVKG | EDIVVVTPILQH | RASQSVSNYLA | DASNRAT | QQRSNWT |
| | | 3231 | 3232 | 3233 | 3234 | 3235 | 3236 |
| 97 | VG9B163 | NHGMS | SITRGGDTTYYPDNVKG | GPLTVGYAMDY | SGNSSNIGHNYVS | DNNQRPS | GIWDSSLSIVV |
| | | 3265 | 3266 | 3267 | 3268 | 3269 | 3270 |
| 98 | VG9B454 | NYYWS | RIYSSGITNYNPSLKS | IVGVKGAFAI | TGTSSDVGGYNYVS | EVSNRPS | SSYTSTSVV |
| | | 3299 | 3300 | 3301 | 3302 | 3303 | 3304 |
| 99 | VG9B439 | NYYWS | RIYSSGITNYNPSLKS | IVGVKGAFAI | TGTSSDVGGYNYVS | EVSNRPS | SSYTSTSVV |
| | | 3333 | 3334 | 3335 | 3336 | 3337 | 3338 |
| 100 | VG9B68 | TYAIS | GIIPIFGTATYAQRFQD | GVGWGSDYYYGLDV | RSSQSLVHSDGNTYLS | KISNRFS | IQATHHPLT |
| | | 3367 | 3368 | 3369 | 3370 | 3371 | 3372 |
| 101 | VG9B449 | NYYWS | RIYSSGITNYNPSLKS | IVGVKGAFAI | TGTSSDVGGYNYVS | EVSNRPS | SSYTSTSVV |
| | | 3401 | 3402 | 3403 | 3404 | 3405 | 3406 |
| 102 | VG9B204 | SFYWS | RIYTSGGTIYNPSLKS | WLRAFDY | SGDKLGDKYAC | QDSKRPS | QAWDSSTVV |
| | | 3435 | 3436 | 3437 | 3438 | 3439 | 3440 |
| 103 | VG9B459 | NYYWS | RIYSSGITNYNPSLKS | IVGVKGAFAI | TGTSSDVGGYNYVS | EVSNRPS | SSYTSTSVV |
| | | 3469 | 3470 | 3471 | 3472 | 3473 | 3474 |
| 104 | VG9B157 | NHGMS | SITRGGDTTYYPDNVKG | GPLTVGYAMDY | QGDSLRSYYAT | GENNRPS | NSRDTGDHHLV |
| | | 3503 | 3504 | 3505 | 3506 | 3507 | 3508 |
| 105 | VG9B453 | NYYWS | RIYSSGITNYNPSLKS | IVGVKGAFAI | TGTSSDVGGYNYVS | EVSNRPS | SSYTSTSVV |
| | | 3537 | 3538 | 3539 | 3540 | 3541 | 3542 |
| 106 | VG9B443 | NYYWS | RIYSSGITNYNPSLKS | IVGVKGAFAI | TGTSSDVGGYNYVS | EVSNRPS | SSYTSTSVV |
| | | 3571 | 3572 | 3573 | 3574 | 3575 | 3576 |
| 107 | VG9B455 | NYYWS | RIYSSGITNYNPSLKS | IVGVKGAFAI | TGTSSDVGGYNYVS | EVSNRPS | SSYTSTSVV |
| | | 3605 | 3606 | 3607 | 3608 | 3609 | 3610 |
| 108 | VG9B466 | NYYWS | RIYSSGITNYNPSLKS | IVGVKGAFAI | TGTSSDVGGYNYVS | EVSNRPS | SSYTSTSVV |
| | | 3639 | 3640 | 3641 | 3642 | 3643 | 3644 |
| 109 | VG9B450 | NYYWS | RIYSSGITNYNPSLKS | IVGVKGAFAI | TGTSSDVGGYNYVS | EVSNRPS | SSYTSTSVV |
| | | 3673 | 3674 | 3675 | 3676 | 3677 | 3678 |
| 110 | VG9B438 | NYYWS | RIYSSGITNYNPSLKS | IVGVKGAFAI | TGTSSDVGGYNYVS | EVSNRPS | SSYTSTSVV |
| | | 3707 | 3708 | 3709 | 3710 | 3711 | 3712 |
| 111 | VG9B464 | NYYWS | RIYSSGITNYNPSLKS | IVGVKGAFAI | TGTSSDVGGYNYVS | EVSNRPS | SSYTSTSVV |
| | | 3741 | 3742 | 3743 | 3744 | 3745 | 3746 |
| 112 | VG9B437 | NYYWS | RIYSSGITNYNPSLKS | IVGVKGAFAI | TGTSSDVGGYNYVS | EVSNRPS | SSYTSTSVV |
| | | 3775 | 3776 | 3777 | 3778 | 3779 | 3780 |
| 113 | VG9B457 | NYYWS | RIYSSGITNYNPSLKS | IVGVKGAFAI | TGTSSDVGGYNYVS | EVSNRPS | SSYTSTSVV |
| | | 3809 | 3810 | 3811 | 3812 | 3813 | 3814 |
| 114 | VG9B442 | NYYWS | RIYSSGITNYNPSLKS | IVGVKGAFAI | TGTSSDVGGYNYVS | EVSNRPS | SSYTSTSVV |
| | | 3843 | 3844 | 3845 | 3846 | 3847 | 3848 |
| 115 | VG9B436 | NYYWS | RIYSSGITNYNPSLKS | IVGVKGAFAI | TGTSSDVGGYNYVS | EVSNRPS | SSYTSTSVV |
| | | 3877 | 3878 | 3879 | 3880 | 3881 | 3882 |
| 116 | VG9B434 | NYYWS | RIYSSGITNYNPSLKS | IVGVKGAFAI | TGTSSDVGGYNYVS | EVSNRPS | SSYTSTSVV |
| | | 3911 | 3912 | 3913 | 3914 | 3915 | 3916 |
| 117 | VG9B460 | NYYWS | RIYSSGITNYNPSLKS | IVGVKGAFAI | TGTSSDVGGYNYVS | EVSNRPS | SSYTSTSVV |
| | | 3945 | 3946 | 3947 | 3948 | 3949 | 3950 |
| 118 | VG9B189 | NHGMS | SITRGGDTTYYPDNVKG | GPLTVGYAMDY | TGTSSDVGGYNYVS | EVSKRPS | NSYAGSNNFEV |
| | | 3979 | 3980 | 3981 | 3982 | 3983 | 3984 |
| 119 | VG9B452 | NYYWS | RIYSSGITNYNPSLKS | IVGVKGAFAI | TGTSSDVGGYNYVS | EVSNRPS | SSYTSTSVV |
| | | 4013 | 4014 | 4015 | 4016 | 4017 | 4018 |
| 120 | VG9B53 | SYYWS | RIHTIGSINYNPSLKS | EGVGATNYYYGMAV | RSSQSLLHSNGYNYLD | LGSNRAS | MQALQTPYT |
| | | 4047 | 4048 | 4049 | 4050 | 4051 | 4052 |
| 121 | VG9B41 | TYWMH | LIDPSDSETRLNQKFKD | NRWLLG | RSSQSLVHSDGNTYLS | KISNRFS | VQTREFPLT |
| | | 4081 | 4082 | 4083 | 4084 | 4085 | 4086 |
| 122 | VG9B136 | SNRAAWN | RTYYRSKWYNEYAASVKS | DLWELREACDI | RASQGISSYLA | VASTLQS | QQLNSYPFT |
| | | 4115 | 4116 | 4117 | 4118 | 4119 | 4120 |
| 123 | VG9B399 | NYDMN | SISSSSSYIYYADSVKG | DVGVTTDYYYYGMDV | RASQSVASSYLA | GASSRAT | QQYGSSPPYT |
| | | 4149 | 4150 | 4151 | 4152 | 4153 | 4154 |
| 124 | VG9B369 | TIGVGVG | LIYWNDDKRYNPSLKS | SHDWVHAFDI | QASQDISHYLN | DASNLDT | QQYDNFPLT |
| | | 4183 | 4184 | 4185 | 4186 | 4187 | 4188 |
| 125 | VG9B144 | GYYMH | WINPNSGATNKEQKFQG | EDDAFDV | QGDNLRNYYAT | GKNNRPS | NSRDSSGNHVV |
| | | 4217 | 4218 | 4219 | 4220 | 4221 | 4222 |
| 126 | VG9B410 | NYGMN | YISSGSSYKYYADSMKG | DPVVTEYYYYGMDV | RASQGINSWLA | AASSLQS | QQANSFPWT |
| | | 4251 | 4252 | 4253 | 4254 | 4255 | 4256 |
| 127 | VG9B386 | SYSMI | SIGSSSTYIYYADSVRG | DGETGGFDY | RASQSISRSYLA | GPSNRAT | QQYGSLPLT |
| | | 4285 | 4286 | 4287 | 4288 | 4289 | 4290 |
| 128 | VG9B125 | SYDMN | YISSSSTAKYYADSVKG | EDIVVVTPILQH | RASQSVSKDLA | DASNRAT | QQRINWPLFT |
| | | 4319 | 4320 | 4321 | 4322 | 4323 | 4324 |
| 129 | VG9B112 | SNSAAWN | GTYYRSKWFNNYAVSVKS | GEWGLRDAFDI | RASQGIHSYLA | VASTLQS | QQLNSYPWT |
| | | 4353 | 4354 | 4355 | 4356 | 4357 | 4358 |
| 130 | VG9B371 | SYDMN | SISSSSSYIYYADSVKG | DRGVGGTDYYYYGLDV | RASQSVSSSYLA | GASSRAT | QQYGSSPPYT |
| | | 4387 | 4388 | 4389 | 4390 | 4391 | 4392 |
| 131 | VG9B387 | GYYLQ | WINPNSGYTDYAQRFQD | LDDAFDV | QGDSLRNYYAI | GKNNRPS | NSRDSSGNHWV |
| | | 4421 | 4422 | 4423 | 4424 | 4425 | 4426 |
| 132 | VG9B47 | NYAIS | GIIPFFGTPDYAQKFQG | GGGYGDYDYYYGINV | RSSQSLVHSDGNTYLS | KISNRFS | MQAKEFPLT |
| | | 4455 | 4456 | 4457 | 4458 | 4459 | 4460 |
| 133 | VG9B379 | GDYIH | WINPNSGGTNYAQKFQG | EGGVAPAAPDAFDI | QGDSLRSYYAR | GKNNRPS | NSRDSSGNHRV |
| | | 4489 | 4490 | 4491 | 4492 | 4493 | 4494 |
| 134 | VG9B447 | NYYWS | RIYSSGITNYNPSLKS | IVGVKGAFAI | TGTSSDVGGYNYVS | EVSNRPS | SSYTSTSVV |
| | | 4523 | 4524 | 4525 | 4526 | 4527 | 4528 |
| 135 | VG9B392 | GYYIH | WINPNSGYTNSAQKFQG | EDDAFDI | QGDSLRSYYAR | GKNNRPS | NSRDNSGNHVV |
| | | 4557 | 4558 | 4559 | 4560 | 4561 | 4562 |
| 136 | VG9B96 | NYAIS | GIIPIFSAGTYAQRFQG | NSGTYYDYYYGMDV | RSSQSLVHSDGNTYLS | KIFNRLS | MQAKQFPLT |
| | | 4591 | 4592 | 4593 | 4594 | 4595 | 4596 |
| 137 | VG9B77 | SYAIS | GIIPFFGTADYAQKFQG | ATVTTDYYYGMDV | RSSQSLVHSDGNTYLS | EISNRFT | MQARQFPLT |
| | | 4625 | 4626 | 4627 | 4628 | 4629 | 4630 |

TABLE 4
Chothia CDR Amino Acid Sequences
(SEQ ID NO disclosed in the cell under each respective sequence)

| | | HC | HC | | | LC | LC |
|---|---|---|---|---|---|---|---|
| | Protein | Chothia | Chothia | HC Chothia | LC Chothia | Chothia | Chothia |
| # | Name | CDR1 | CDR2 | CDR3 | CDR1 | CDR2 | CDR3 |
| 1 | VG9B54 | GFAFSSY | SSSSSY | DGELGPFD | SQSVSSSY | GAS | YGRSPL |
| | | 7 | 8 | 9 | 10 | | 12 |
| 2 | VG9B121 | GFTFSDY | SSTSNY | SYNWNYGGAFD | SQSVSSSY | GAS | YGRSPL |
| | | 41 | 42 | 43 | 44 | | 46 |
| 3 | VG9B429 | GFTFSNY | SSSSSY | DVGVTTDYYYYGMD | SQGINSW | AAS | ANSFPW |
| | | 75 | 76 | 77 | 78 | | 80 |
| 4 | VG9B370 | RFTLSSY | SSSSSY | DRGVGGTDYYYYGLD | SQSVSSSY | GAS | YGSSPPY |
| | | 109 | 110 | 111 | 112 | | 114 |
| 5 | VG9B80 | GFTFSTY | SSSSSY | DGELGVFD | SQSISSSY | GPS | FGRSPL |
| | | 143 | 144 | 145 | 146 | | 148 |
| 6 | VG9B414 | GFTFSNY | SSSSSY | DVGVTTDYYYYGMD | SQGINSW | AAS | ANSFPW |
| | | 177 | 178 | 179 | 180 | | 182 |
| 7 | VG9B195 | GFTFSPY | SSSSSY | DGDLVGPTYYFD | DKLGDKY | QHN | WDSTTV |
| | | 211 | 212 | 213 | 214 | | 216 |
| 8 | VG9B140 | GFTFRSY | STSSGY | DRGIAVAGDYYYGMD | SQSVSSSY | GAS | YGSSPPY |
| | | 245 | 246 | 247 | 248 | | 250 |
| 9 | VG9B426 | GFTFSNY | SSSSSY | DVGVTTDYYYYGMD | SQSVASSY | GAS | YGSSPPY |
| | | 279 | 280 | 281 | 282 | | 284 |
| 10 | VG9B46 | GFTFSIN | SSTSDY | DDVFGAFD | SQSVSRSY | GSS | YSRSPL |
| | | 313 | 314 | 315 | 316 | | 318 |
| 11 | VG9B416 | GFTFSNY | SSSSSY | DVGVTTDYYYYGMD | SQSVASSY | GAS | YGSSPPY |
| | | 347 | 348 | 349 | 350 | | 352 |
| 12 | VG9B69 | GFTFSSY | TSSSYY | DLGVRGVDYYYYGLD | SQSVSSSY | GAS | YGSSPPY |
| | | 381 | 382 | 383 | 384 | | 386 |
| 13 | VG9B415 | GFTFSNY | SSSSSY | DVGVTTDYYYYGMD | SQSVASSY | GAS | YGSSPPY |
| | | 415 | 416 | 417 | 418 | | 420 |
| 14 | VG9B104 | GFTFSVY | GSSSSY | DHDYGGLD | SQSVSSSY | GPS | YGRSPL |
| | | 449 | 450 | 451 | 452 | | 454 |
| 15 | VG9B198 | GDSISSI | YTTDI | NGYSYGGFN | SSSNIGAGYD | GDS | YDSSLSVV |
| | | 483 | 484 | 485 | 486 | | 488 |
| 16 | VG9B463 | GFTFSNY | SSSSSY | DVGVTTDYYYYGMD | SSSNIGSNT | TNT | WDDSLNAW |
| | | 517 | 518 | 519 | 520 | | 522 |
| 17 | VG9B469 | GFTFSHY | SSSSSY | DRGVGDTSDYYSFGLD | SQNVSSTY | GAC | YGSSPPY |
| | | 551 | 552 | 553 | 554 | | 556 |
| 18 | VG9B428 | GFTFSNY | SSGSSY | DPVVTEYYYYGMD | SQGINSW | AAS | ANSFPW |
| | | 585 | 586 | 587 | 588 | | 590 |
| 19 | VG9B430 | GFTFSSY | SSSSSY | DVGVTTDYYYYGMD | SQSVASSY | GAS | YGSSPPY |
| | | 619 | 620 | 621 | 622 | | 624 |
| 20 | VG9B423 | GFTFSNY | SSSSSY | DVGVTTDYYYYGMD | SQSVASSY | GAS | YGSSPPY |
| | | 653 | 654 | 655 | 656 | | 658 |
| 21 | VG9B98 | GFTFSSY | GSSSTY | DGELGPFE | SQSVSTSY | GTS | YGRSPL |
| | | 687 | 688 | 689 | 690 | | 692 |
| 22 | VG9B73 | GFTFSNY | SSSSSY | EIGVTGTTYYQDYGMD | SQSFSSNY | GAS | YGSSPPF |
| | | 721 | 722 | 723 | 724 | | 726 |
| 23 | VG9B133 | GFTFSNY | SSSSSY | DLGITGTTMDYYYGMD | SQSVSSSY | GAS | YGSSPPY |
| | | 755 | 756 | 757 | 758 | | 760 |
| 24 | VG9B368 | GFTFNSY | SSSSSY | DRGIGGDYYSYAMD | SQSVSSSY | GAS | YGSSPPY |
| | | 789 | 790 | 791 | 792 | | 794 |
| 25 | VG9B424 | GFTFSNY | SSSSSY | DVGVTTDYYYYGMD | SQSVASSY | GAS | YGSSPPY |
| | | 823 | 824 | 825 | 826 | | 828 |
| 26 | VG9B427 | GFTFSNY | SSSSSY | DVGVTTDYYYYGMD | SQSVASSY | GAS | YGSSPPY |
| | | 857 | 858 | 859 | 860 | | 862 |
| 27 | VG9B417 | GFTFSNY | SSGSSY | DPVVTEYYYYGMD | SQGINSW | AAS | ANSFPW |
| | | 891 | 892 | 893 | 894 | | 896 |
| 28 | VG9B58 | GGSIKSS | FYLGS | ERPVLDAFD | SQDITNY | AAS | HDTYPY |
| | | 925 | 926 | 927 | 928 | | 930 |
| 29 | VG9B419 | GFTFSNY | SSSSSY | DVGVTTDYYYYGMD | SQSVASSY | GAS | YGSSPPY |
| | | 959 | 960 | 961 | 962 | | 964 |
| 30 | VG9B425 | GFTFSNY | SSSSSY | DVGVTTDYYYYGMD | SQSVASSY | GAS | YGSSPPY |
| | | 993 | 994 | 995 | 996 | | 998 |
| 31 | VG9B143 | GFTFSTY | STSSYT | EGDWWYFD | TSSDVGGYNY | EVS | YTSSSTL |
| | | 1027 | 1028 | 1029 | 1030 | | 1032 |
| 32 | VG9B418 | GFTFSNY | SSSSSY | DVGVTTDYYYYGMD | SQSVASSY | GAS | YGSSPPY |
| | | 1061 | 1062 | 1063 | 1064 | | 1066 |
| 33 | VG9B472 | GFTFSHY | SSSSSY | DRGVGDTSDYYSFGLD | SQGIATY | AAS | YNSAPPW |
| | | 1095 | 1096 | 1097 | 1098 | | 1100 |
| 34 | VG9B421 | GFTFSNY | SSGSSY | DPVVTEYYYYGMD | SQGINSW | AAS | ANSFPW |
| | | 1129 | 1130 | 1131 | 1132 | | 1134 |
| 35 | VG9B88 | GFTFSSY | STSSSY | DGDMVAPIKGSFD | SQSLLNSDDGNTY | TLS | RIEFPI |
| | | 1163 | 1164 | 1165 | 1166 | | 1168 |
| 36 | VG9B384 | GFSLSSSGM | DWFDD | IRGTGAYYYGLD | NELGDKY | QDN | WDSSKV |
| | | 1197 | 1198 | 1199 | 1200 | | 1202 |
| 37 | VG9B413 | GFTFSNY | SSSSSY | DVGVTTDYYYYGMD | SQSVASSY | GAS | YGSSPPY |
| | | 1231 | 1232 | 1233 | 1234 | | 1236 |
| 38 | VG9B36 | GFTFSSY | STGSSY | DKGLAVTGYIMD | SQDISNY | DAS | YDNLPMY |
| | | 1265 | 1266 | 1267 | 1268 | | 1270 |
| 39 | VG9B403 | GFTFSNY | SSSSSY | DVGVTTDYYYYGMD | SQSVASSY | GAS | YGSSPPY |
| | | 1299 | 1300 | 1301 | 1302 | | 1304 |
| 40 | VG9B191 | GFTFSRY | SSSSSY | DGPTVNWDYYFD | SSSNIGNNY | DNN | WDSSLSTSV |
| | | 1333 | 1334 | 1335 | 1336 | | 1338 |
| 41 | VG9B44 | GDSVSSNSA | YYRSKWY | ESGSYYTDGFD | SQGISSY | AAS | FNSYPY |
| | | 1367 | 1368 | 1369 | 1370 | | 1372 |
| 42 | VG9B67 | GFTFSSY | SSSSSY | DGDILATIRGSFD | SQSLLDSDAGNTY | TLS | RIEFPI |
| | | 1401 | 1402 | 1403 | 1404 | | 1406 |
| 43 | VG9B402 | GFTFSNY | SSGSSY | DPVVTEYYYYGMD | SQGINSW | AAS | ANSFPW |
| | | 1435 | 1436 | 1437 | 1438 | | 1440 |
| 44 | VG9B127 | GFTFGAY | SSSSSY | DGDIVSTIRGSFD | SQSLLDSDDGNTY | TLS | RIEFPI |
| | | 1469 | 1470 | 1471 | 1472 | | 1474 |
| 45 | VG9B137 | GITFSSY | GSSSSY | SYSWNYGGAFD | SQSVSSSY | GAS | YGSSPPY |
| | | 1503 | 1504 | 1505 | 1506 | | 1508 |
| 46 | VG9B33 | GFTFSSY | STSSSY | DTSVTKYPDTFD | SQDINNY | GAS | YNSAPF |
| | | 1537 | 1538 | 1539 | 1540 | | 1542 |
| 47 | VG9B162 | GYTFTGD | NPNSGY | EGDAFD | TSSDVGGYNY | EVS | YAGSNNFE |
| | | 1571 | 1572 | 1573 | 1574 | | 1576 |
| 48 | VG9B152 | GFTFSSY | SSSSDY | DWELLGFD | DKLGDKY | QHN | WDSTTV |
| | | 1605 | 1606 | 1607 | 1608 | | 1610 |
| 49 | VG9B64 | GFSLSISGV | YWNDD | SGQWLEGDAFD | SESLVHSDGNTY | KIS | ATQFPL |
| | | 1639 | 1640 | 1641 | 1642 | | 1644 |
| 50 | VG9B21 | GFSFSNY | SSSSHY | DRGVTTDYYYYALD | SQGIYNY | AAS | YNRAPF |
| | | 1673 | 1674 | 1675 | 1676 | | 1678 |
| 51 | VG9B128 | GFTFGTY | WYNGSN | GGFGESFD | SQSVIDY | DAS | RSNWPL |
| | | 1707 | 1708 | 1709 | 1710 | | 1712 |
| 52 | VG9B66 | GFTFSTY | SSSSFY | DGDIVATIRGSFD | SQDITNF | TAS | YNSAPF |
| | | 1741 | 1742 | 1743 | 1744 | | 1746 |
| 53 | VG9B32 | GFTFSRY | SGTGYT | DQEPGFD | GQSLVHSDGNTY | KIS | ATHFPF |
| | | 1775 | 1776 | 1777 | 1778 | | 1780 |
| 54 | VG9B57 | GFTFSSH | SSSSSY | DLGVGVRDYYYYGMD | SQDITNY | AAS | HDTYPY |
| | | 1809 | 1810 | 1811 | 1812 | | 1814 |
| 55 | VG9B135 | GFSLTTYGV | YWNDD | DYDFWSGYFD | SQSVSSSY | GAS | YGSSPL |
| | | 1843 | 1844 | 1845 | 1846 | | 1848 |
| 56 | VG9B60 | GYTFTGY | NPNSGV | GGSLVRGVISGLD | SQSFSGSY | GAS | YGSSPPY |
| | | 1877 | 1878 | 1879 | 1880 | | 1882 |
| 57 | VG9B409 | GYTLTSY | NPSGGS | GSYGWYFD | SQSVTSSY | GAS | YGSSPPY |
| | | 1911 | 1912 | 1913 | 1914 | | 1916 |
| 58 | VG9B411 | GFTFSNY | SSSSSY | DVGVTTDYYYYGMD | SQGINSW | AAS | YNNWPR |
| | | 1945 | 1946 | 1947 | 1948 | | 1950 |
| 59 | VG9B129 | GGTFSSY | LPILST | AHDYYYGMD | SQSVSNNY | GAS | YGSSPPY |
| | | 1979 | 1980 | 1981 | 1982 | | 1984 |
| 60 | VG9B396 | GDSIRGY | FTTGN | EKWDSSSSALYFD | SSSNIGADYD | GNT | YDSRLTGYV |
| | | 2013 | 2014 | 2015 | 2016 | | 2018 |
| 61 | VG9B470 | GFSLTTSGV | LWNDH | DKWELRDAFD | SQGISRY | VAS | LISYPY |
| | | 2047 | 2048 | 2049 | 2050 | | 2052 |
| 62 | VG9B111 | GFTFSTY | SSDSSY | DSVTGPFD | SQDISHY | DSY | YDNLPY |
| | | 2081 | 2082 | 2083 | 2084 | | 2086 |
| 63 | VG9B169 | GGSITNY | FYSGS | VGRWELRTAFD | SSSNIGSNT | SNN | WDDSLNGPG |
| | | 2115 | 2116 | 2117 | 2118 | | 2120 |
| 64 | VG9B639 | GFTFTTY | SSSSIY | ERVYTVSFD | SQDISHY | DSY | YDNLPY |
| | | 2149 | 2150 | 2151 | 2152 | | 2154 |
| 65 | VG9B201 | GFSLTTHGV | YWNAD | EGDWGHYFD | SSSNIGNND | DNN | WDSSLSAIW |
| | | 2183 | 2184 | 2185 | 2186 | | 2188 |
| 66 | VG9B161 | GGSISSY | FYSGS | VGRWELRGAFD | SSSNIGSNT | SNN | WDDSLNSPG |
| | | 2217 | 2218 | 2219 | 2220 | | 2222 |
| 67 | VG9B383 | SGSISSY | YTIGN | EGYYDSSGSFFPGAFG | TSNDVGSYNL | AGS | FAGATNV |
| | | 2251 | 2252 | 2253 | 2254 | | 2256 |
| 68 | VG9B382 | GDSISGY | FTTGN | ERWDSSSSALYFD | SSSNIGADYD | GNS | YDSSMSGYV |
| | | 2285 | 2286 | 2287 | 2288 | | 2290 |
| 69 | VG9B156 | NGSISGY | FYSGS | LGKWELRTAFD | STSNIGSDT | SNN | WDDSLNGP |
| | | 2319 | 2320 | 2321 | 2322 | | 2324 |
| 70 | VG9B205 | NGSISGY | FYSGS | LGKWELRTAFD | DKLGDKY | QDS | WDSSTV |
| | | 2353 | 2354 | 2355 | 2356 | | 2358 |
| 71 | VG9B86 | GDSISSS | YSSGS | YSGSYWYFD | SQSVSSY | DTS | RSDWLL |
| | | 2387 | 2388 | 2389 | 2390 | | 2392 |
| 72 | VG9B154 | GGSISYY | YYSGS | EGKWELRTTFD | SSSNIGSNT | SNN | WDDSLNGP |
| | | 2421 | 2422 | 2423 | 2424 | | 2426 |
| 73 | VG9B159 | GGSISSY | YTGGR | DMEYYYDRSGYSYWYFD | NNIGSKS | DDS | WDSSSDHV |
| | | 2455 | 2456 | 2457 | 2458 | | 2460 |
| 74 | VG9B465 | GGSITSY | YTIGN | EGYYESDGSFFPGAFN | TSNDVGSYNL | AGS | YAGTTNV |
| | | 2489 | 2490 | 2491 | 2492 | | 2494 |
| 75 | VG9B194 | GYTFTGD | NPNSGY | ELDALD | TSSDVGGYNY | EVS | YAGSNNFE |
| | | 2523 | 2524 | 2525 | 2526 | | 2528 |
| 76 | VG9B182 | GDSINNH | FYNGN | VGRWVLRTAFD | SSSNIGSNT | SNN | WDDSLNGPG |
| | | 2557 | 2558 | 2559 | 2560 | | 2562 |
| 77 | VG9B173 | GGSINNY | YYSGS | EGKWELRSAFD | SSSNIESNT | SNN | WDDSLNGP |
| | | 2591 | 2592 | 2593 | 2594 | | 2596 |
| 78 | VG9B87 | GYTFTSE | NPSGGS | ERGYSYGSFD | SQGISSY | AAS | FNSYSL |
| | | 2625 | 2626 | 2627 | 2628 | | 2630 |
| 79 | VG9B208 | GGSISGY | FYSGS | VGKWELRSSFD | STSNIGNND | DNN | WDSSLSVW |
| | | 2659 | 2660 | 2661 | 2662 | | 2664 |
| 80 | VG9B372 | GGSISNGGF | NYSGS | DRNYEWNFD | SQSVSSY | DAS | RSNWPL |
| | | 2693 | 2694 | 2695 | 2696 | | 2698 |
| 81 | VG9B186 | GFSLTNIRM | FSNDE | MRLPYGMD | STSNIGSNT | SNN | WDDSLNGP |
| | | 2727 | 2728 | 2729 | 2730 | | 2732 |
| 82 | VG9B177 | GGSINNY | FYSGS | VGKWELRTAFD | TSSDVADYNY | EVS | YTSSFTV |
| | | 2761 | 2762 | 2763 | 2764 | | 2766 |
| 83 | VG9B114 | GFTFSRY | GSAGD | GKWELRDAFD | SQGIHSY | VAS | LNSYPY |
| | | 2795 | 2796 | 2797 | 2798 | | 2800 |
| 84 | VG9B147 | GGSISGY | FYSGS | EGKWELRSTFD | DSLRSYY | GEN | RDTGDHHL |
| | | 2829 | 2830 | 2831 | 2832 | | 2834 |
| 85 | VG9B65 | GGTFNIY | IPFFGT | GGDSGYDWGFD | SQSLVHSDGNTY | QIS | ATQFPL |
| | | 2863 | 2864 | 2865 | 2866 | | 2868 |
| 86 | VG9B81 | GGSINTY | YTSDN | YNWNYWYFD | SQGISSY | TAS | LNSYPY |
| | | 2897 | 2898 | 2899 | 2900 | | 2902 |
| 87 | VG9B203 | GDSIGHY | YTSGS | SGGNFYWYFD | SSSNIGNNY | DNN | WDSSLSAG |
| | | 2931 | 2932 | 2933 | 2934 | | 2936 |
| 88 | VG9B380 | GGSISNY | YPSGI | IMGTKGAFD | TSSDVGGYNY | EVS | YTSTSV |
| | | 2965 | 2966 | 2967 | 2968 | | 2970 |
| 89 | VG9B103 | GGTFSSY | IPIFGT | GVGWGTDYYYGLD | SQGLVHSDGNTY | KIS | ATHHPL |
| | | 2999 | 3000 | 3001 | 3002 | | 3004 |
| 90 | VG9B462 | GGSISNY | YSSGI | IVGVKGAFA | TSSDVGGYNY | EVS | YTSTSV |
| | | 3033 | 3034 | 3035 | 3036 | | 3038 |
| 91 | VG9B461 | GGSISNY | YSSGI | IVGVKGAFA | TSSDVGGYNY | EVS | YTSTSV |
| | | 3067 | 3068 | 3069 | 3070 | | 3072 |
| 92 | VG9B106 | GYTFTGY | NPSSGD | ELGIGVFD | SQSVLYSSNNKNY | WAS | YYSIPY |
| | | 3101 | 3102 | 3103 | 3104 | | 3106 |
| 93 | VG9B115 | GFSLSTYGV | YWNDD | ESDWSYYFD | SQSVSSY | DAS | RSSWPW |
| | | 3135 | 3136 | 3137 | 3138 | | 3140 |
| 94 | VG9B27 | GGTFSSY | LPILST | AHDYYYGMD | SQSLVHSDGNTY | KIS | ATHHPL |
| | | 3169 | 3170 | 3171 | 3172 | | 3174 |
| 95 | VG9B458 | GGSISNY | YSSGI | IVGVKGAFA | TSSDVGGYNY | EVS | YTSTSV |
| | | 3203 | 3204 | 3205 | 3206 | | 3208 |
| 96 | VG9B131 | GFTFSSY | SSSSTA | EDIVVVTPILQ | SQSVSNY | DAS | RSNW |
| | | 3237 | 3238 | 3239 | 3240 | | 3242 |
| 97 | VG9B163 | GFTFSNH | TRGGDT | GPLTVGYAMD | NSSNIGHNY | DNN | WDSSLSIV |
| | | 3271 | 3272 | 3273 | 3274 | | 3276 |
| 98 | VG9B454 | GGSISNY | YSSGI | IVGVKGAFA | TSSDVGGYNY | EVS | YTSTSV |
| | | 3305 | 3306 | 3307 | 3308 | | 3310 |
| 99 | VG9B439 | GGSISNY | YSSGI | IVGVKGAFA | TSSDVGGYNY | EVS | YTSTSV |
| | | 3339 | 3340 | 3341 | 3342 | | 3344 |
| 100 | VG9B68 | GGTFSTY | IPIFGT | GVGWGSDYYYGLD | SQSLVHSDGNTY | KIS | ATHHPL |
| | | 3373 | 3374 | 3375 | 3376 | | 3378 |
| 101 | VG9B449 | GGSISNY | YSSGI | IVGVKGAFA | TSSDVGGYNY | EVS | YTSTSV |
| | | 3407 | 3408 | 3409 | 3410 | | 3412 |
| 102 | VG9B204 | YGSFSSF | YTSGG | WLRAFD | DKLGDKY | QDS | WDSSTV |
| | | 3441 | 3442 | 3443 | 3444 | | 3446 |
| 103 | VG9B459 | GGSISNY | YSSGI | IVGVKGAFA | TSSDVGGYNY | EVS | YTSTSV |
| | | 3475 | 3476 | 3477 | 3478 | | 3480 |
| 104 | VG9B157 | GFTFSNH | TRGGDT | GPLTVGYAMD | DSLRSYY | GEN | RDTGDHHL |
| | | 3509 | 3510 | 3511 | 3512 | | 3514 |
| 105 | VG9B453 | GGSISNY | YSSGI | IVGVKGAFA | TSSDVGGYNY | EVS | YTSTSV |
| | | 3543 | 3544 | 3545 | 3546 | | 3548 |
| 106 | VG9B443 | GGSISNY | YSSGI | IVGVKGAFA | TSSDVGGYNY | EVS | YTSTSV |
| | | 3577 | 3578 | 3579 | 3580 | | 3582 |
| 107 | VG9B455 | GGSISNY | YSSGI | IVGVKGAFA | TSSDVGGYNY | EVS | YTSTSV |
| | | 3611 | 3612 | 3613 | 3614 | | 3616 |
| 108 | VG9B466 | GGSISNY | YSSGI | IVGVKGAFA | TSSDVGGYNY | EVS | YTSTSV |
| | | 3645 | 3646 | 3647 | 3648 | | 3650 |
| 109 | VG9B450 | GGSISNY | YSSGI | IVGVKGAFA | TSSDVGGYNY | EVS | YTSTSV |
| | | 3679 | 3680 | 3681 | 3682 | | 3684 |
| 110 | VG9B438 | GGSISNY | YSSGI | IVGVKGAFA | TSSDVGGYNY | EVS | YTSTSV |
| | | 3713 | 3714 | 3715 | 3716 | | 3718 |
| 111 | VG9B464 | GGSISNY | YSSGI | IVGVKGAFA | TSSDVGGYNY | EVS | YTSTSV |
| | | 3747 | 3748 | 3749 | 3750 | | 3752 |
| 112 | VG9B437 | GGSISNY | YSSGI | IVGVKGAFA | TSSDVGGYNY | EVS | YTSTSV |
| | | 3781 | 3782 | 3783 | 3784 | | 3786 |
| 113 | VG9B457 | GGSISNY | YSSGI | IVGVKGAFA | TSSDVGGYNY | EVS | YTSTSV |
| | | 3815 | 3816 | 3817 | 3818 | | 3820 |
| 114 | VG9B442 | GGSISNY | YSSGI | IVGVKGAFA | TSSDVGGYNY | EVS | YTSTSV |
| | | 3849 | 3850 | 3851 | 3852 | | 3854 |
| 115 | VG9B436 | GGSISNY | YSSGI | IVGVKGAFA | TSSDVGGYNY | EVS | YTSTSV |
| | | 3883 | 3884 | 3885 | 3886 | | 3888 |
| 116 | VG9B434 | GGSISNY | YSSGI | IVGVKGAFA | TSSDVGGYNY | EVS | YTSTSV |
| | | 3917 | 3918 | 3919 | 3920 | | 3922 |
| 117 | VG9B460 | GGSISNY | YSSGI | IVGVKGAFA | TSSDVGGYNY | EVS | YTSTSV |
| | | 3951 | 3952 | 3953 | 3954 | | 3956 |
| 118 | VG9B189 | GFTFSNH | TRGGDT | GPLTVGYAMD | TSSDVGGYNY | EVS | YAGSNNFE |
| | | 3985 | 3986 | 3987 | 3988 | | 3990 |
| 119 | VG9B452 | GGSISNY | YSSGI | IVGVKGAFA | TSSDVGGYNY | EVS | YTSTSV |
| | | 4019 | 4020 | 4021 | 4022 | | 4024 |
| 120 | VG9B53 | GGSISSY | HTIGS | EGVGATNYYYGMA | SQSLLHSNGYNY | LGS | ALQTPY |
| | | 4053 | 4054 | 4055 | 4056 | | 4058 |
| 121 | VG9B41 | GYSFTTY | DPSDSE | NRWLL | SQSLVHSDGNTY | KIS | TREFPL |
| | | 4087 | 4088 | 4089 | 4090 | | 4092 |
| 122 | VG9B136 | GDSVSSNRA | YYRSKWY | DLWELREACD | SQGISSY | VAS | LNSYPF |
| | | 4121 | 4122 | 4123 | 4124 | | 4126 |
| 123 | VG9B399 | GFTFSNY | SSSSSY | DVGVTTDYYYYGMD | SQSVASSY | GAS | YGSSPPY |
| | | 4155 | 4156 | 4157 | 4158 | | 4160 |
| 124 | VG9B369 | GFSLSTIGV | YWNDD | SHDWVHAFD | SQDISHY | DAS | YDNFPL |
| | | 4189 | 4190 | 4191 | 4192 | | 4194 |
| 125 | VG9B144 | GYTFTGY | NPNSGA | EDDAFD | DNLRNYY | GKN | RDSSGNHV |
| | | 4223 | 4224 | 4225 | 4226 | | 4228 |
| 126 | VG9B410 | GFTFSNY | SSGSSY | DPVVTEYYYYGMD | SQGINSW | AAS | ANSFPW |
| | | 4257 | 4258 | 4259 | 4260 | | 4262 |
| 127 | VG9B386 | GFTFSSY | GSSSTY | DGETGGFD | SQSISRSY | GPS | YGSLPL |
| | | 4291 | 4292 | 4293 | 4294 | | 4296 |
| 128 | VG9B125 | GFTFSSY | SSSSTA | EDIVVVTPILQ | SQSVSKD | DAS | RINWPLF |
| | | 4325 | 4326 | 4327 | 4328 | | 4330 |
| 129 | VG9B112 | GDSVSSNSA | YYRSKWF | GEWGLRDAFD | SQGIHSY | VAS | LNSYPW |
| | | 4359 | 4360 | 4361 | 4362 | | 4364 |
| 130 | VG9B371 | RFTLSSY | SSSSSY | DRGVGGTDYYYYGLD | SQSVSSSY | GAS | YGSSPPY |
| | | 4393 | 4394 | 4395 | 4396 | | 4398 |
| 131 | VG9B387 | GFTFTGY | NPNSGY | LDDAFD | DSLRNYY | GKN | RDSSGNHW |
| | | 4427 | 4428 | 4429 | 4430 | | 4432 |
| 132 | VG9B47 | GGSFSNY | IPFFGT | GGGYGDYDYYYGIN | SQSLVHSDGNTY | KIS | AKEFPL |
| | | 4461 | 4462 | 4463 | 4464 | | 4466 |
| 133 | VG9B379 | GYTFTGD | NPNSGG | EGGVAPAAPDAFD | DSLRSYY | GKN | RDSSGNHR |
| | | 4495 | 4496 | 4497 | 4498 | | 4500 |
| 134 | VG9B447 | GGSISNY | YSSGI | IVGVKGAFA | TSSDVGGYNY | EVS | YTSTSV |
| | | 4529 | 4530 | 4531 | 4532 | | 4534 |
| 135 | VG9B392 | GYTFTGY | NPNSGY | EDDAFD | DSLRSYY | GKN | RDNSGNHV |
| | | 4563 | 4564 | 4565 | 4566 | | 4568 |
| 136 | VG9B96 | GGTFSNY | IPIFSA | NSGTYYDYYYGMD | SQSLVHSDGNTY | KIF | AKQFPL |
| | | 4597 | 4598 | 4599 | 4600 | | 4602 |
| 137 | VG9B77 | GGTFSSY | IPFFGT | ATVTTDYYYGMD | SQSLVHSDGNTY | EIS | ARQFPL |
| | | 4631 | 4632 | 4633 | 4634 | | 4636 |

TABLE 5
AbM CDR Amino Acid Sequences
(SEQ ID NO disclosed in the cell under each respective sequence)

| | Protein | HC AbM | HC AbM | | | LC AbM | LC AbM |
|---|---|---|---|---|---|---|---|
| # | Name | CDR1 | CDR2 | HC AbM CDR3 | LC AbM CDR1 | CDR2 | CDR3 |
| 1 | VG9B54 | GFAFSSYSLT | SISSSSSYIF | DGELGPFDY | RASQSVSSSYLA | GASSRAT | QQYGRSPLT |
| | | 13 | 14 | 15 | 16 | 17 | 18 |
| 2 | VG9B121 | GFTFSDYAMD | SISSTSNYIF | SYNWNYGGAFDI | RASQSVSSSYFA | GASSRAT | QQYGRSPLT |
| | | 47 | 48 | 49 | 50 | 51 | 52 |
| 3 | VG9B429 | GFTFSNYDMN | SISSSSSYIY | DVGVTTDYYYYGMDV | RASQGINSWLA | AASSLQS | QQANSFPWT |
| | | 81 | 82 | 83 | 84 | 85 | 86 |
| 4 | VG9B370 | RFTLSSYDMN | SISSSSSYIY | DRGVGGTDYYYYGLDV | RASQSVSSSYLA | GASSRAT | QQYGSSPPYT |
| | | 115 | 116 | 117 | 118 | 119 | 120 |
| 5 | VG9B80 | GFTFSTYSMN | SISSSSSYIF | DGELGVFDY | RASQSISSSYLA | GPSGRAT | QQFGRSPLT |
| | | 149 | 150 | 151 | 152 | 153 | 154 |
| 6 | VG9B414 | GFTFSNYDMN | SISSSSSYIY | DVGVTTDYYYYGMDV | RASQGINSWLA | AASSLQS | QQANSFPWT |
| | | 183 | 184 | 185 | 186 | 187 | 188 |
| 7 | VG9B195 | GFTFSPYTMN | SISSSSSYMY | DGDLVGPTYYFDY | SGDKLGDKYAC | QHNKRPS | QAWDSTTVV |
| | | 217 | 218 | 219 | 220 | 221 | 222 |
| 8 | VG9B140 | GFTFRSYDMN | SISTSSGYIY | DRGIAVAGDYYYGMDV | RASQSVSSSYLA | GASSRAT | QQYGSSPPYT |
| | | 251 | 252 | 253 | 254 | 255 | 256 |
| 9 | VG9B426 | GFTFSNYDMN | SISSSSSYIY | DVGVTTDYYYYGMDV | RASQSVASSYLA | GASSRAT | QQYGSSPPYT |
| | | 285 | 286 | 287 | 288 | 289 | 290 |
| 10 | VG9B46 | GFTFSINSMN | SISSTSDYIF | DDVFGAFDI | RTSQSVSRSYLG | GSSSRAT | QQYSRSPLT |
| | | 319 | 320 | 321 | 322 | 323 | 324 |
| 11 | VG9B416 | GFTFSNYDMN | SISSSSSYIY | DVGVTTDYYYYGMDV | RASQSVASSYLA | GASSRAT | QQYGSSPPYT |
| | | 353 | 354 | 355 | 356 | 357 | 358 |
| 12 | VG9B69 | GFTFSSYDIN | SITSSSYYIY | DLGVRGVDYYYYGLDV | RASQSVSSSYLA | GASSRAT | QQYGSSPPYT |
| | | 387 | 388 | 389 | 390 | 391 | 392 |
| 13 | VG9B415 | GFTFSNYDMN | SISSSSSYIY | DVGVTTDYYYYGMDV | RASQSVASSYLA | GASSRAT | QQYGSSPPYT |
| | | 421 | 422 | 423 | 424 | 425 | 426 |
| 14 | VG9B104 | GFTFSVYSMN | SIGSSSSYIF | DHDYGGLDY | RASQSVSSSYLA | GPSNRAT | QQYGRSPLT |
| | | 455 | 456 | 457 | 458 | 459 | 460 |
| 15 | VG9B198 | GDSISSIYWS | RIYTTDITN | NGYSYGGFNY | TGSSSNIGAGYDVH | GDSYRPS | QSYDSSLSVVV |
| | | 489 | 490 | 491 | 492 | 493 | 494 |
| 16 | VG9B463 | GFTFSNYDMN | SISSSSSYIY | DVGVTTDYYYYGMDV | SGSSSNIGSNTVN | TNTQRPS | AAWDDSLNAWV |
| | | 523 | 524 | 525 | 526 | 527 | 528 |
| 17 | VG9B469 | GFTFSHYDMN | SISSSSSYIF | DRGVGDTSDYYSFGLDV | RASQNVSSTYLA | GACSRAT | QQYGSSPPYT |
| | | 557 | 558 | 559 | 560 | 561 | 562 |
| 18 | VG9B428 | GFTFSNYGMN | YISSGSSYKY | DPVVTEYYYYGMDV | RASQGINSWLA | AASSLQS | QQANSFPWT |
| | | 591 | 592 | 593 | 594 | 595 | 596 |
| 19 | VG9B430 | GFTFSSYDMN | SISSSSSYIY | DVGVTTDYYYYGMDV | RASQSVASSYLA | GASSRAT | QQYGSSPPYT |
| | | 625 | 626 | 627 | 628 | 629 | 630 |
| 20 | VG9B423 | GFTFSNYDMN | SISSSSSYIY | DVGVTTDYYYYGMDV | RASQSVASSYLA | GASSRAT | QQYGSSPPYT |
| | | 659 | 660 | 661 | 662 | 663 | 664 |
| 21 | VG9B98 | GFTFSSYSMY | SIGSSSTYIF | DGELGPFEY | RASQSVSTSYLA | GTSSRAT | LQYGRSPLT |
| | | 693 | 694 | 695 | 696 | 697 | 698 |
| 22 | VG9B73 | GFTFSNYDMN | SISSSSSYIY | EIGVTGTTYYQDYGMDV | RASQSFSSNYLA | GASSRAT | QQYGSSPPFT |
| | | 727 | 728 | 729 | 730 | 731 | 732 |
| 23 | VG9B133 | GFTFSNYDMN | SISSSSSYIY | DLGITGTTMDYYYGMDV | RASQSVSSSYLA | GASSRAT | QQYGSSPPYT |
| | | 761 | 762 | 763 | 764 | 765 | 766 |
| 24 | VG9B368 | GFTFNSYDMN | SISSSSSYIY | DRGIGGDYYSYAMDV | RASQSVSSSYLA | GASSRAT | QQYGSSPPYT |
| | | 795 | 796 | 797 | 798 | 799 | 800 |
| 25 | VG9B424 | GFTFSNYDMN | SISSSSSYIY | DVGVTTDYYYYGMDV | RASQSVASSYLA | GASSRAT | QQYGSSPPYT |
| | | 829 | 830 | 831 | 832 | 833 | 834 |
| 26 | VG9B427 | GFTFSNYDMN | SISSSSSYIY | DVGVTTDYYYYGMDV | RASQSVASSYLA | GASSRAT | QQYGSSPPYT |
| | | 863 | 864 | 865 | 866 | 867 | 868 |
| 27 | VG9B417 | GFTFSNYGMN | YISSGSSYKY | DPVVTEYYYYGMDV | RASQGINSWLA | AASSLQS | QQANSFPWT |
| | | 897 | 898 | 899 | 900 | 901 | 902 |
| 28 | VG9B58 | GGSIKSSYWT | YMFYLGSTN | ERPVLDAFDI | RASQDITNYLA | AASSLQG | LQHDTYPYT |
| | | 931 | 932 | 933 | 934 | 935 | 936 |
| 29 | VG9B419 | GFTFSNYDMN | SISSSSSYIY | DVGVTTDYYYYGMDV | RASQSVASSYLA | GASSRAT | QQYGSSPPYT |
| | | 965 | 966 | 967 | 968 | 969 | 970 |
| 30 | VG9B425 | GFTFSNYDMN | SISSSSSYIY | DVGVTTDYYYYGMDV | RASQSVASSYLA | GASSRAT | QQYGSSPPYT |
| | | 999 | 1000 | 1001 | 1002 | 1003 | 1004 |
| 31 | VG9B143 | GFTFSTYGMN | YISTSSYTIY | EGDWWYFDL | TGTSSDVGGYNYVS | EVSNRPS | SSYTSSSTLV |
| | | 1033 | 1034 | 1035 | 1036 | 1037 | 1038 |
| 32 | VG9B418 | GFTFSNYDMN | SISSSSSYIY | DVGVTTDYYYYGMDV | RASQSVASSYLA | GASSRAT | QQYGSSPPYT |
| | | 1067 | 1068 | 1069 | 1070 | 1071 | 1072 |
| 33 | VG9B472 | GFTFSHYDMN | SISSSSSYIF | DRGVGDTSDYYSFGLDV | RASQGIATYLA | AASTLQS | QKYNSAPPWT |
| | | 1101 | 1102 | 1103 | 1104 | 1105 | 1106 |
| 34 | VG9B421 | GFTFSNYGMN | YISSGSSYKY | DPVVTEYYYYGMDV | RASQGINSWLA | AASSLQS | QQANSFPWT |
| | | 1135 | 1136 | 1137 | 1138 | 1139 | 1140 |
| 35 | VG9B88 | GFTFSSYTMN | SISTSSSYID | DGDMVAPIKGSFDY | RSSQSLLNSDDGNTYLD | TLSYRAS | MQRIEFPIT |
| | | 1169 | 1170 | 1171 | 1172 | 1173 | 1174 |
| 36 | VG9B384 | GFSLSSSGMCVS | LIDWFDDKY | IRGTGAYYYGLDV | SGNELGDKYAS | QDNKRPS | QAWDSSKVV |
| | | 1203 | 1204 | 1205 | 1206 | 1207 | 1208 |
| 37 | VG9B413 | GFTFSNYDMN | SISSSSSYIY | DVGVTTDYYYYGMDV | RASQSVASSYLA | GASSRAT | QQYGSSPPYT |
| | | 1237 | 1238 | 1239 | 1240 | 1241 | 1242 |
| 38 | VG9B36 | GFTFSSYGMY | SISTGSSYIY | DKGLAVTGYIMDV | QASQDISNYLN | DASNLET | QQYDNLPMYT |
| | | 1271 | 1272 | 1273 | 1274 | 1275 | 1276 |
| 39 | VG9B403 | GFTFSNYDMN | SISSSSSYIY | DVGVTTDYYYYGMDV | RASQSVASSYLA | GASSRAT | QQYGSSPPYT |
| | | 1305 | 1306 | 1307 | 1308 | 1309 | 1310 |
| 40 | VG9B191 | GFTFSRYSMN | SISSSSSYIY | DGPTVNWDYYFDL | SGSSSNIGNNYVS | DNNKRPS | GTWDSSLSTSVV |
| | | 1339 | 1340 | 1341 | 1342 | 1343 | 1344 |
| 41 | VG9B44 | GDSVSSNSAAWN | RTYYRSKWYND | ESGSYYTDGFDI | RASQGISSYLA | AASTLQS | QEFNSYPYT |
| | | 1373 | 1374 | 1375 | 1376 | 1377 | 1378 |
| 42 | VG9B67 | GFTFSSYTMN | SISSSSSYID | DGDILATIRGSFDY | RSSQSLLDSDAGNTYLD | TLSYRAS | MQRIEFPIT |
| | | 1407 | 1408 | 1409 | 1410 | 1411 | 1412 |
| 43 | VG9B402 | GFTFSNYGMN | YISSGSSYKY | DPVVTEYYYYGMDV | RASQGINSWLA | AASSLQS | QQANSFPWT |
| | | 1441 | 1442 | 1443 | 1444 | 1445 | 1446 |
| 44 | VG9B127 | GFTFGAYTMN | SISSSSSYID | DGDIVSTIRGSFDY | RSSQSLLDSDDGNTYLD | TLSYRAS | MQRIEFPIT |
| | | 1475 | 1476 | 1477 | 1478 | 1479 | 1480 |
| 45 | VG9B137 | GITFSSYSMD | SIGSSSSYIF | SYSWNYGGAFDI | RASQSVSSSYLA | GASSRAT | QQYGSSPPYT |
| | | 1509 | 1510 | 1511 | 1512 | 1513 | 1514 |
| 46 | VG9B33 | GFTFSSYNMN | SISTSSSYIY | DTSVTKYPDTFDI | RASQDINNYLA | GASTLQS | QKYNSAPFT |
| | | 1543 | 1544 | 1545 | 1546 | 1547 | 1548 |
| 47 | VG9B162 | GYTFTGDYMH | WINPNSGYTN | EGDAFDV | TGTSSDVGGYNYVS | EVSKRPS | NSYAGSNNFEV |
| | | 1577 | 1578 | 1579 | 1580 | 1581 | 1582 |
| 48 | VG9B152 | GFTFSSYSMI | SISSSSDYIY | DWELLGFDC | SGDKLGDKYAC | QHNKRPS | QAWDSTTVV |
| | | 1611 | 1612 | 1613 | 1614 | 1615 | 1616 |
| 49 | VG9B64 | GFSLSISGVSVG | LIYWNDDKR | SGQWLEGDAFDI | RSSESLVHSDGNTYLS | KISNRFS | MQATQFPLT |
| | | 1645 | 1646 | 1647 | 1648 | 1649 | 1650 |
| 50 | VG9B21 | GFSFSNYDMN | SISSSSHYIY | DRGVTTDYYYYALDV | RASQGIYNYLA | AASTLQS | QKYNRAPFT |
| | | 1679 | 1680 | 1681 | 1682 | 1683 | 1684 |
| 51 | VG9B128 | GFTFGTYGMH | VIWYNGSNKY | GGFGESFDS | RASQSVIDYLA | DASNRAT | QQRSNWPLT |
| | | 1713 | 1714 | 1715 | 1716 | 1717 | 1718 |
| 52 | VG9B66 | GFTFSTYTMN | SISSSSFYMD | DGDIVATIRGSFDY | RASQDITNFLA | TASTLQS | QKYNSAPFT |
| | | 1747 | 1748 | 1749 | 1750 | 1751 | 1752 |
| 53 | VG9B32 | GFTFSRYAMN | FISGTGYTVY | DQEPGFDY | RSGQSLVHSDGNTYLS | KISNRFS | MQATHFPFT |
| | | 1781 | 1782 | 1783 | 1784 | 1785 | 1786 |
| 54 | VG9B57 | GFTFSSHDMN | SISSSSSYIF | DLGVGVRDYYYYGMDV | RASQDITNYLA | AASSLQG | LQHDTYPYT |
| | | 1815 | 1816 | 1817 | 1818 | 1819 | 1820 |
| 55 | VG9B135 | GFSLTTYGVGVG | LIYWNDDKR | DYDFWSGYFDY | RASQSVSSSYLA | GASSRAT | QQYGSSPLT |
| | | 1849 | 1850 | 1851 | 1852 | 1853 | 1854 |
| 56 | VG9B60 | GYTFTGYYMH | RINPNSGVTH | GGSLVRGVISGLDY | RASQSFSGSYLA | GASSRAT | QQYGSSPPYT |
| | | 1883 | 1884 | 1885 | 1886 | 1887 | 1888 |
| 57 | VG9B409 | GYTLTSYYMH | IINPSGGSTS | GSYGWYFDL | RASQSVTSSYLA | GASSRAT | QQYGSSPPYT |
| | | 1917 | 1918 | 1919 | 1920 | 1921 | 1922 |
| 58 | VG9B411 | GFTFSNYDMN | SISSSSSYIY | DVGVTTDYYYYGMDV | RASQGINSWLA | AASTLQS | QQYNNWPRT |
| | | 1951 | 1952 | 1953 | 1954 | 1955 | 1956 |
| 59 | VG9B129 | GGTFSSYVIS | GILPILSTAN | AHDYYYGMDV | RASQSVSNNYLA | GASSRAT | QQYGSSPPYT |
| | | 1985 | 1986 | 1987 | 1988 | 1989 | 1990 |
| 60 | VG9B396 | GDSIRGYYWN | RIFTTGNTN | EKWDSSSSALYFDF | TGSSSNIGADYDVK | GNTDRPS | QSYDSRLTGYVV |
| | | 2019 | 2020 | 2021 | 2022 | 2023 | 2024 |
| 61 | VG9B470 | GFSLTTSGVGVG | LILWNDHTI | DKWELRDAFDI | RASQGISRYLA | VASTLQS | QQLISYPYT |
| | | 2053 | 2054 | 2055 | 2056 | 2057 | 2058 |
| 62 | VG9B111 | GFTFSTYSVN | SISSDSSYIF | DSVTGPFDY | QASQDISHYLN | DSYILET | QQYDNLPYT |
| | | 2087 | 2088 | 2089 | 2090 | 2091 | 2092 |
| 63 | VG9B169 | GGSITNYFWN | YIFYSGSTS | VGRWELRTAFDI | SGSSSNIGSNTVN | SNNQRPS | AAWDDSLNGPGV |
| | | 2121 | 2122 | 2123 | 2124 | 2125 | 2126 |
| 64 | VG9B639 | GFTFTTYRMN | SISSSSIYIH | ERVYTVSFDY | QASQDISHYLN | DSYILET | QQYDNLPYT |
| | | 2155 | 2156 | 2157 | 2158 | 2159 | 2160 |
| 65 | VG9B201 | GFSLTTHGVGVG | LIYWNADKH | EGDWGHYFDF | SGSSSNIGNNDVS | DNNRRPS | ETWDSSLSAIWV |
| | | 2189 | 2190 | 2191 | 2192 | 2193 | 2194 |
| 66 | VG9B161 | GGSISSYFWS | YIFYSGSTN | VGRWELRGAFDI | SGSSSNIGSNTVN | SNNQRPS | AAWDDSLNSPGV |
| | | 2223 | 2224 | 2225 | 2226 | 2227 | 2228 |
| 67 | VG9B383 | SGSISSYYWN | RIYTIGNTN | EGYYDSSGSFFPGAFGI | TGTSNDVGSYNLVS | AGSKRPS | CSFAGATNVV |
| | | 2257 | 2258 | 2259 | 2260 | 2261 | 2262 |
| 68 | VG9B382 | GDSISGYYWN | RIFTTGNTN | ERWDSSSSALYFDY | TGSSSNIGADYDIK | GNSNRPS | QSYDSSMSGYVV |
| | | 2291 | 2292 | 2293 | 2294 | 2295 | 2296 |
| 69 | VG9B156 | NGSISGYFWN | YIFYSGSTN | LGKWELRTAFDI | SGSTSNIGSDTVN | SNNQRPS | AAWDDSLNGPV |
| | | 2325 | 2326 | 2327 | 2328 | 2329 | 2330 |
| 70 | VG9B205 | NGSISGYFWN | YIFYSGSTN | LGKWELRTAFDI | SGDKLGDKYAC | QDSKRPS | QAWDSSTVV |
| | | 2359 | 2360 | 2361 | 2362 | 2363 | 2364 |
| 71 | VG9B86 | GDSISSSYWS | RFYSSGSTS | YSGSYWYFDL | RASQSVSSYLA | DTSNRAT | QQRSDWLLT |
| | | 2393 | 2394 | 2395 | 2396 | 2397 | 2398 |
| 72 | VG9B154 | GGSISYYFWN | YIYYSGSTN | EGKWELRTTFDI | SGSSSNIGSNTVN | SNNQRPS | AAWDDSLNGPV |
| | | 2427 | 2428 | 2429 | 2430 | 2431 | 2432 |
| 73 | VG9B159 | GGSISSYYWS | RFYTGGRNN | DMEYYYDRSGYSYWYFDL | GGNNIGSKSVH | DDSDRPS | QVWDSSSDHVV |
| | | 2461 | 2462 | 2463 | 2464 | 2465 | 2466 |
| 74 | VG9B465 | GGSITSYYWN | RIYTIGNTN | EGYYESDGSFFPGAFNI | TGTSNDVGSYNLVS | AGSKRPS | CSYAGTTNVV |
| | | 2495 | 2496 | 2497 | 2498 | 2499 | 2500 |
| 75 | VG9B194 | GYTFTGDYMH | WINPNSGYTN | ELDALDV | TGTSSDVGGYNYVS | EVSKRPS | NSYAGSNNFEV |
| | | 2529 | 2530 | 2531 | 2532 | 2533 | 2534 |
| 76 | VG9B182 | GDSINNHFWS | FVFYNGNTN | VGRWVLRTAFDI | SGSSSNIGSNTVN | SNNQRPS | AAWDDSLNGPGV |
| | | 2563 | 2564 | 2565 | 2566 | 2567 | 2568 |
| 77 | VG9B173 | GGSINNYFWS | YIYYSGSTN | EGKWELRSAFDI | SGSSSNIESNTVN | SNNQRPS | TAWDDSLNGPV |
| | | 2597 | 2598 | 2599 | 2600 | 2601 | 2602 |
| 78 | VG9B87 | GYTFTSEYIH | IINPSGGSTS | ERGYSYGSFDY | RASQGISSYLA | AASTLQS | QQFNSYSLT |
| | | 2631 | 2632 | 2633 | 2634 | 2635 | 2636 |
| 79 | VG9B208 | GGSISGYYWS | YIFYSGSIN | VGKWELRSSFDI | SGSTSNIGNNDVS | DNNKRPS | GTWDSSLSVWV |
| | | 2665 | 2666 | 2667 | 2668 | 2669 | 2670 |
| 80 | VG9B372 | GGSISNGGFYWS | YINYSGSTY | DRNYEWNFDL | RASQSVSSYLA | DASNRAT | QQRSNWPLT |
| | | 2699 | 2700 | 2701 | 2702 | 2703 | 2704 |
| 81 | VG9B186 | GFSLTNIRMSVS | HIFSNDEKS | MRLPYGMDV | SGSTSNIGSNTVN | SNNQRPS | AAWDDSLNGPV |
| | | 2733 | 2734 | 2735 | 2736 | 2737 | 2738 |
| 82 | VG9B177 | GGSINNYFWS | YIFYSGSTN | VGKWELRTAFDI | TGTSSDVADYNYVS | EVSNRPS | CSYTSSFTVV |
| | | 2767 | 2768 | 2769 | 2770 | 2771 | 2772 |
| 83 | VG9B114 | GFTFSRYDMH | AIGSAGDTY | GKWELRDAFDI | RASQGIHSYLA | VASTLQS | QQLNSYPYT |
| | | 2801 | 2802 | 2803 | 2804 | 2805 | 2806 |
| 84 | VG9B147 | GGSISGYFWN | YIFYSGSTN | EGKWELRSTFDI | QGDSLRSYYAT | GENNRPS | NSRDTGDHHLV |
| | | 2835 | 2836 | 2837 | 2838 | 2839 | 2840 |
| 85 | VG9B65 | GGTFNIYAIN | GIIPFFGTAN | GGDSGYDWGFDY | RSSQSLVHSDGNTYLS | QISNRFS | MQATQFPLT |
| | | 2869 | 2870 | 2871 | 2872 | 2873 | 2874 |
| 86 | VG9B81 | GGSINTYYWS | RIYTSDNTN | YNWNYWYFDL | RASQGISSYLA | TASTLQS | QHLNSYPYT |
| | | 2903 | 2904 | 2905 | 2906 | 2907 | 2908 |
| 87 | VG9B203 | GDSIGHYYWN | RIYTSGSTN | SGGNFYWYFDL | SGSSSNIGNNYVS | DNNKRPS | GTWDSSLSAGV |
| | | 2937 | 2938 | 2939 | 2940 | 2941 | 2942 |
| 88 | VG9B380 | GGSISNYYWS | RIYPSGITS | IMGTKGAFDI | TGTSSDVGGYNYVS | EVSNRPS | SSYTSTSVV |
| | | 2971 | 2972 | 2973 | 2974 | 2975 | 2976 |
| 89 | VG9B103 | GGTFSSYAIS | GIIPIFGTAT | GVGWGTDYYYGLDV | RSSQGLVHSDGNTYLS | KISNRFS | MQATHHPLT |
| | | 3005 | 3006 | 3007 | 3008 | 3009 | 3010 |
| 90 | VG9B462 | GGSISNYYWS | RIYSSGITN | IVGVKGAFAI | TGTSSDVGGYNYVS | EVSNRPS | SSYTSTSVV |
| | | 3039 | 3040 | 3041 | 3042 | 3043 | 3044 |
| 91 | VG9B461 | GGSISNYYWS | RIYSSGITN | IVGVKGAFAI | TGTSSDVGGYNYVS | EVSNRPS | SSYTSTSVV |
| | | 3073 | 3074 | 3075 | 3076 | 3077 | 3078 |
| 92 | VG9B106 | GYTFTGYYLH | WINPSSGDTD | ELGIGVFDY | KSSQSVLYSSNNKNYLG | WASTRES | QQYYSIPYT |
| | | 3107 | 3108 | 3109 | 3110 | 3111 | 3112 |
| 93 | VG9B115 | GFSLSTYGVGVG | LIYWNDDKR | ESDWSYYFDY | RASQSVSSYLA | DASNRAT | QQRSSWPWT |
| | | 3141 | 3142 | 3143 | 3144 | 3145 | 3146 |
| 94 | VG9B27 | GGTFSSYVIS | GILPILSTAN | AHDYYYGMDV | RSSQSLVHSDGNTYLS | KISNRFS | MQATHHPLT |
| | | 3175 | 3176 | 3177 | 3178 | 3179 | 3180 |
| 95 | VG9B458 | GGSISNYYWS | RIYSSGITN | IVGVKGAFAI | TGTSSDVGGYNYVS | EVSNRPS | SSYTSTSVV |
| | | 3209 | 3210 | 3211 | 3212 | 3213 | 3214 |
| 96 | VG9B131 | GFTFSSYDMN | YISSSSTAKY | EDIVVVTPILQH | RASQSVSNYLA | DASNRAT | QQRSNWT |
| | | 3243 | 3244 | 3245 | 3246 | 3247 | 3248 |
| 97 | VG9B163 | GFTFSNHGMS | SITRGGDTTY | GPLTVGYAMDY | SGNSSNIGHNYVS | DNNQRPS | GIWDSSLSIVV |
| | | 3277 | 3278 | 3279 | 3280 | 3281 | 3282 |
| 98 | VG9B454 | GGSISNYYWS | RIYSSGITN | IVGVKGAFAI | TGTSSDVGGYNYVS | EVSNRPS | SSYTSTSVV |
| | | 3311 | 3312 | 3313 | 3314 | 3315 | 3316 |
| 99 | VG9B439 | GGSISNYYWS | RIYSSGITN | IVGVKGAFAI | TGTSSDVGGYNYVS | EVSNRPS | SSYTSTSVV |
| | | 3345 | 3346 | 3347 | 3348 | 3349 | 3350 |
| 100 | VG9B68 | GGTFSTYAIS | GIIPIFGTAT | GVGWGSDYYYGLDV | RSSQSLVHSDGNTYLS | KISNRFS | IQATHHPLT |
| | | 3379 | 3380 | 3381 | 3382 | 3383 | 3384 |
| 101 | VG9B449 | GGSISNYYWS | RIYSSGITN | IVGVKGAFAI | TGTSSDVGGYNYVS | EVSNRPS | SSYTSTSVV |
| | | 3413 | 3414 | 3415 | 3416 | 3417 | 3418 |
| 102 | VG9B204 | YGSFSSFYWS | RIYTSGGTI | WLRAFDY | SGDKLGDKYAC | QDSKRPS | QAWDSSTVV |
| | | 3447 | 3448 | 3449 | 3450 | 3451 | 3452 |
| 103 | VG9B459 | GGSISNYYWS | RIYSSGITN | IVGVKGAFAI | TGTSSDVGGYNYVS | EVSNRPS | SSYTSTSVV |
| | | 3481 | 3482 | 3483 | 3484 | 3485 | 3486 |
| 104 | VG9B157 | GFTFSNHGMS | SITRGGDTTY | GPLTVGYAMDY | QGDSLRSYYAT | GENNRPS | NSRDTGDHHLV |
| | | 3515 | 3516 | 3517 | 3518 | 3519 | 3520 |
| 105 | VG9B453 | GGSISNYYWS | RIYSSGITN | IVGVKGAFAI | TGTSSDVGGYNYVS | EVSNRPS | SSYTSTSVV |
| | | 3549 | 3550 | 3551 | 3552 | 3553 | 3554 |
| 106 | VG9B443 | GGSISNYYWS | RIYSSGITN | IVGVKGAFAI | TGTSSDVGGYNYVS | EVSNRPS | SSYTSTSVV |
| | | 3583 | 3584 | 3585 | 3586 | 3587 | 3588 |
| 107 | VG9B455 | GGSISNYYWS | RIYSSGITN | IVGVKGAFAI | TGTSSDVGGYNYVS | EVSNRPS | SSYTSTSVV |
| | | 3617 | 3618 | 3619 | 3620 | 3621 | 3622 |
| 108 | VG9B466 | GGSISNYYWS | RIYSSGITN | IVGVKGAFAI | TGTSSDVGGYNYVS | EVSNRPS | SSYTSTSVV |
| | | 3651 | 3652 | 3653 | 3654 | 3655 | 3656 |
| 109 | VG9B450 | GGSISNYYWS | RIYSSGITN | IVGVKGAFAI | TGTSSDVGGYNYVS | EVSNRPS | SSYTSTSVV |
| | | 3685 | 3686 | 3687 | 3688 | 3689 | 3690 |
| 110 | VG9B438 | GGSISNYYWS | RIYSSGITN | IVGVKGAFAI | TGTSSDVGGYNYVS | EVSNRPS | SSYTSTSVV |
| | | 3719 | 3720 | 3721 | 3722 | 3723 | 3724 |
| 111 | VG9B464 | GGSISNYYWS | RIYSSGITN | IVGVKGAFAI | TGTSSDVGGYNYVS | EVSNRPS | SSYTSTSVV |
| | | 3753 | 3754 | 3755 | 3756 | 3757 | 3758 |
| 112 | VG9B437 | GGSISNYYWS | RIYSSGITN | IVGVKGAFAI | TGTSSDVGGYNYVS | EVSNRPS | SSYTSTSVV |
| | | 3787 | 3788 | 3789 | 3790 | 3791 | 3792 |
| 113 | VG9B457 | GGSISNYYWS | RIYSSGITN | IVGVKGAFAI | TGTSSDVGGYNYVS | EVSNRPS | SSYTSTSVV |
| | | 3821 | 3822 | 3823 | 3824 | 3825 | 3826 |
| 114 | VG9B442 | GGSISNYYWS | RIYSSGITN | IVGVKGAFAI | TGTSSDVGGYNYVS | EVSNRPS | SSYTSTSVV |
| | | 3855 | 3856 | 3857 | 3858 | 3859 | 3860 |
| 115 | VG9B436 | GGSISNYYWS | RIYSSGITN | IVGVKGAFAI | TGTSSDVGGYNYVS | EVSNRPS | SSYTSTSVV |
| | | 3889 | 3890 | 3891 | 3892 | 3893 | 3894 |
| 116 | VG9B434 | GGSISNYYWS | RIYSSGITN | IVGVKGAFAI | TGTSSDVGGYNYVS | EVSNRPS | SSYTSTSVV |
| | | 3923 | 3924 | 3925 | 3926 | 3927 | 3928 |
| 117 | VG9B460 | GGSISNYYWS | RIYSSGITN | IVGVKGAFAI | TGTSSDVGGYNYVS | EVSNRPS | SSYTSTSVV |
| | | 3957 | 3958 | 3959 | 3960 | 3961 | 3962 |
| 118 | VG9B189 | GFTFSNHGMS | SITRGGDTTY | GPLTVGYAMDY | TGTSSDVGGYNYVS | EVSKRPS | NSYAGSNNFEV |
| | | 3991 | 3992 | 3993 | 3994 | 3995 | 3996 |
| 119 | VG9B452 | GGSISNYYWS | RIYSSGITN | IVGVKGAFAI | TGTSSDVGGYNYVS | EVSNRPS | SSYTSTSVV |
| | | 4025 | 4026 | 4027 | 4028 | 4029 | 4030 |
| 120 | VG9B53 | GGSISSYYWS | RIHTIGSIN | EGVGATNYYYGMAV | RSSQSLLHSNGYNYLD | LGSNRAS | MQALQTPYT |
| | | 4059 | 4060 | 4061 | 4062 | 4063 | 4064 |
| 121 | VG9B41 | GYSFTTYWMH | LIDPSDSETR | NRWLLG | RSSQSLVHSDGNTYLS | KISNRFS | VQTREFPLT |
| | | 4093 | 4094 | 4095 | 4096 | 4097 | 4098 |
| 122 | VG9B136 | GDSVSSNRAAWN | RTYYRSKWYNE | DLWELREACDI | RASQGISSYLA | VASTLQS | QQLNSYPFT |
| | | 4127 | 4128 | 4129 | 4130 | 4131 | 4132 |
| 123 | VG9B399 | GFTFSNYDMN | SISSSSSYIY | DVGVTTDYYYYGMDV | RASQSVASSYLA | GASSRAT | QQYGSSPPYT |
| | | 4161 | 4162 | 4163 | 4164 | 4165 | 4166 |
| 124 | VG9B369 | GFSLSTIGVGVG | LIYWNDDKR | SHDWVHAFDI | QASQDISHYLN | DASNLDT | QQYDNFPLT |
| | | 4195 | 4196 | 4197 | 4198 | 4199 | 4200 |
| 125 | VG9B144 | GYTFTGYYMH | WINPNSGATN | EDDAFDV | QGDNLRNYYAT | GKNNRPS | NSRDSSGNHVV |
| | | 4229 | 4230 | 4231 | 4232 | 4233 | 4234 |
| 126 | VG9B410 | GFTFSNYGMN | YISSGSSYKY | DPVVTEYYYYGMDV | RASQGINSWLA | AASSLQS | QQANSFPWT |
| | | 4263 | 4264 | 4265 | 4266 | 4267 | 4268 |
| 127 | VG9B386 | GFTFSSYSMI | SIGSSSTYIY | DGETGGFDY | RASQSISRSYLA | GPSNRAT | QQYGSLPLT |
| | | 4297 | 4298 | 4299 | 4300 | 4301 | 4302 |
| 128 | VG9B125 | GFTFSSYDMN | YISSSSTAKY | EDIVVVTPILQH | RASQSVSKDLA | DASNRAT | QQRINWPLFT |
| | | 4331 | 4332 | 4333 | 4334 | 4335 | 4336 |
| 129 | VG9B112 | GDSVSSNSAAWN | GTYYRSKWFNN | GEWGLRDAFDI | RASQGIHSYLA | VASTLQS | QQLNSYPWT |
| | | 4365 | 4366 | 4367 | 4368 | 4369 | 4370 |
| 130 | VG9B371 | RFTLSSYDMN | SISSSSSYIY | DRGVGGTDYYYYGLDV | RASQSVSSSYLA | GASSRAT | QQYGSSPPYT |
| | | 4399 | 4400 | 4401 | 4402 | 4403 | 4404 |
| 131 | VG9B387 | GFTFTGYYLQ | WINPNSGYTD | LDDAFDV | QGDSLRNYYAI | GKNNRPS | NSRDSSGNHWV |
| | | 4433 | 4434 | 4435 | 4436 | 4437 | 4438 |
| 132 | VG9B47 | GGSFSNYAIS | GIIPFFGTPD | GGGYGDYDYYYGINV | RSSQSLVHSDGNTYLS | KISNRFS | MQAKEFPLT |
| | | 4467 | 4468 | 4469 | 4470 | 4471 | 4472 |
| 133 | VG9B379 | GYTFTGDYIH | WINPNSGGTN | EGGVAPAAPDAFDI | QGDSLRSYYAR | GKNNRPS | NSRDSSGNHRV |
| | | 4501 | 4502 | 4503 | 4504 | 4505 | 4506 |
| 134 | VG9B447 | GGSISNYYWS | RIYSSGITN | IVGVKGAFAI | TGTSSDVGGYNYVS | EVSNRPS | SSYTSTSVV |
| | | 4535 | 4536 | 4537 | 4538 | 4539 | 4540 |
| 135 | VG9B392 | GYTFTGYYIH | WINPNSGYTN | EDDAFDI | QGDSLRSYYAR | GKNNRPS | NSRDNSGNHVV |
| | | 4569 | 4570 | 4571 | 4572 | 4573 | 4574 |
| 136 | VG9B96 | GGTFSNYAIS | GIIPIFSAGT | NSGTYYDYYYGMDV | RSSQSLVHSDGNTYLS | KIFNRLS | MQAKQFPLT |
| | | 4603 | 4604 | 4605 | 4606 | 4607 | 4608 |
| 137 | VG9B77 | GGTFSSYAIS | GIIPFFGTAD | ATVTTDYYYGMDV | RSSQSLVHSDGNTYLS | EISNRFT | MQARQFPLT |
| | | 4637 | 4638 | 4639 | 4640 | 4641 | 4642 |

TABLE 6
Contact CDR Amino Acid Sequences
(SEQ ID NO disclosed in the cell under each respective sequence)

| | Protein | HC Contact | HC Contact | HC Contact | LC Contact | LC Contact | LC Contact |
|---|---|---|---|---|---|---|---|
| # | Name | CDR1 | CDR2 | CDR3 | CDR1 | CDR2 | CDR3 |
| 1 | VG9B54 | SSYSLT | WVSSISSSSSYIF | ATDGELGPFD | SSSYLAWF | RLIYGASSRA | QQYGRSPL |
| | | 19 | 20 | 21 | 22 | 23 | 24 |
| 2 | VG9B121 | SDYAMD | WISSISSTSNYIF | ANSYNWNYGGAFD | SSSYFAWY | LLIYGASSRA | QQYGRSPL |
| | | 53 | 54 | 55 | 56 | 57 | 58 |
| 3 | VG9B429 | SNYDMN | WVSSISSSSSYIY | ARDVGVTTDYYYYGMD | NSWLAWY | LLIYAASSLQ | QQANSFPW |
| | | 87 | 88 | 89 | 90 | 91 | 92 |
| 4 | VG9B370 | SSYDMN | WVSSISSSSSYIY | ARDRGVGGTDYYYYGLD | SSSYLAWY | LLIYGASSRA | QQYGSSPPY |
| | | 121 | 122 | 123 | 124 | 125 | 126 |
| 5 | VG9B80 | STYSMN | WVSSISSSSSYIF | AKDGELGVFD | SSSYLAWY | VLIYGPSGRA | QQFGRSPL |
| | | 155 | 156 | 157 | 158 | 159 | 160 |
| 6 | VG9B414 | SNYDMN | WVSSISSSSSYIY | ARDVGVTTDYYYYGMD | NSWLAWY | LLIYAASSLQ | QQANSFPW |
| | | 189 | 190 | 191 | 192 | 193 | 194 |
| 7 | VG9B195 | SPYTMN | WVSSISSSSSYMY | ARDGDLVGPTYYFD | DKYACWY | LVIYQHNKRP | QAWDSTTV |
| | | 223 | 224 | 225 | 226 | 227 | 228 |
| 8 | VG9B140 | RSYDMN | WVSSISTSSGYIY | ARDRGIAVAGDYYYGMD | SSSYLAWY | LLIYGASSRA | QQYGSSPPY |
| | | 257 | 258 | 259 | 260 | 261 | 262 |
| 9 | VG9B426 | SNYDMN | WVSSISSSSSYIY | ARDVGVTTDYYYYGMD | ASSYLAWY | LLIYGASSRA | QQYGSSPPY |
| | | 291 | 292 | 293 | 294 | 295 | 296 |
| 10 | VG9B46 | SINSMN | WVSSISSTSDYIF | VRDDVFGAFD | SRSYLGWY | LLIFGSSSRA | QQYSRSPL |
| | | 325 | 326 | 327 | 328 | 329 | 330 |
| 11 | VG9B416 | SNYDMN | WVSSISSSSSYIY | ARDVGVTTDYYYYGMD | ASSYLAWY | LLIYGASSRA | QQYGSSPPY |
| | | 359 | 360 | 361 | 362 | 363 | 364 |
| 12 | VG9B69 | SSYDIN | WVSSITSSSYYIY | ARDLGVRGVDYYYYGLD | SSSYLAWY | LLIYGASSRA | QQYGSSPPY |
| | | 393 | 394 | 395 | 396 | 397 | 398 |
| 13 | VG9B415 | SNYDMN | WVSSISSSSSYIY | ARDVGVTTDYYYYGMD | ASSYLAWY | LLIYGASSRA | QQYGSSPPY |
| | | 427 | 428 | 429 | 430 | 431 | 432 |
| 14 | VG9B104 | SVYSMN | WVSSIGSSSSYIF | GRDHDYGGLD | SSSYLAWY | LLIYGPSNRA | QQYGRSPL |
| | | 461 | 462 | 463 | 464 | 465 | 466 |
| 15 | VG9B198 | SSIYWS | WIGRIYTTDITN | AKNGYSYGGFN | IGAGYDVHWY | LLIYGDSYRP | QSYDSSLSVV |
| | | 495 | 496 | 497 | 498 | 499 | 500 |
| 16 | VG9B463 | SNYDMN | WVSSISSSSSYIY | ARDVGVTTDYYYYGMD | IGSNTVNWY | LLIYTNTQRP | AAWDDSLNAW |
| | | 529 | 530 | 531 | 532 | 533 | 534 |
| 17 | VG9B469 | SHYDMN | WVSSISSSSSYIF | ARDRGVGDTSDYYSFGLD | SSTYLAWY | LLIYGACSRA | QQYGSSPPY |
| | | 563 | 564 | 565 | 566 | 567 | 568 |
| 18 | VG9B428 | SNYGMN | WVSYISSGSSYKY | ARDPVVTEYYYYGMD | NSWLAWY | LLIYAASSLQ | QQANSFPW |
| | | 597 | 598 | 599 | 600 | 601 | 602 |
| 19 | VG9B430 | SSYDMN | WVSSISSSSSYIY | ARDVGVTTDYYYYGMD | ASSYLAWY | LLIYGASSRA | QQYGSSPPY |
| | | 631 | 632 | 633 | 634 | 635 | 636 |
| 20 | VG9B423 | SNYDMN | WVSSISSSSSYIY | ARDVGVTTDYYYYGMD | ASSYLAWY | LLIYGASSRA | QQYGSSPPY |
| | | 665 | 666 | 667 | 668 | 669 | 670 |
| 21 | VG9B98 | SSYSMY | WVSSIGSSSTYIF | ARDGELGPFE | STSYLAWF | LLIFGTSSRA | LQYGRSPL |
| | | 699 | 700 | 701 | 702 | 703 | 704 |
| 22 | VG9B73 | SNYDMN | WVSSISSSSSYIY | AREIGVTGITYYQDYGMD | SSNYLAWY | LLIYGASSRA | QQYGSSPPF |
| | | 733 | 734 | 735 | 736 | 737 | 738 |
| 23 | VG9B133 | SNYDMN | WVSSISSSSSYIY | ARDLGITGTTMDYYYGMD | SSSYLAWY | LLIYGASSRA | QQYGSSPPY |
| | | 767 | 768 | 769 | 770 | 771 | 772 |
| 24 | VG9B368 | NSYDMN | WVSSISSSSSYIY | ARDRGIGGDYYSYAMD | SSSYLAWY | LLIYGASSRA | QQYGSSPPY |
| | | 801 | 802 | 803 | 804 | 805 | 806 |
| 25 | VG9B424 | SNYDMN | WVSSISSSSSYIY | ARDVGVTTDYYYYGMD | ASSYLAWY | LLIYGASSRA | QQYGSSPPY |
| | | 835 | 836 | 837 | 838 | 839 | 840 |
| 26 | VG9B427 | SNYDMN | WVSSISSSSSYIY | ARDVGVTTDYYYYGMD | ASSYLAWY | LLIYGASSRA | QQYGSSPPY |
| | | 869 | 870 | 871 | 872 | 873 | 874 |
| 27 | VG9B417 | SNYGMN | WVSYISSGSSYKY | ARDPVVTEYYYYGMD | NSWLAWY | LLIYAASSLQ | QQANSFPW |
| | | 903 | 904 | 905 | 906 | 907 | 908 |
| 28 | VG9B58 | KSSYWT | WIGYMFYLGSTN | SRERPVLDAFD | TNYLAWF | RLIYAASSLQ | LQHDTYPY |
| | | 937 | 938 | 939 | 940 | 941 | 942 |
| 29 | VG9B419 | SNYDMN | WVSSISSSSSYIY | ARDVGVTTDYYYYGMD | ASSYLAWY | LLIYGASSRA | QQYGSSPPY |
| | | 971 | 972 | 973 | 974 | 975 | 976 |
| 30 | VG9B425 | SNYDMN | WVSSISSSSSYIY | ARDVGVTTDYYYYGMD | ASSYLAWY | LLIYGASSRA | QQYGSSPPY |
| | | 1005 | 1006 | 1007 | 1008 | 1009 | 1010 |
| 31 | VG9B143 | STYGMN | WISYISTSSYTIY | AREGDWWYFD | VGGYNYVSWY | LMIYEVSNRP | SSYTSSSTL |
| | | 1039 | 1040 | 1041 | 1042 | 1043 | 1044 |
| 32 | VG9B418 | SNYDMN | WVSSISSSSSYIY | ARDVGVTTDYYYYGMD | ASSYLAWY | LLIYGASSRA | QQYGSSPPY |
| | | 1073 | 1074 | 1075 | 1076 | 1077 | 1078 |
| 33 | VG9B472 | SHYDMN | WVSSISSSSSYIF | ARDRGVGDTSDYYSFGLD | ATYLAWY | LLIYAASTLQ | QKYNSAPPW |
| | | 1107 | 1108 | 1109 | 1110 | 1111 | 1112 |
| 34 | VG9B421 | SNYGMN | WVSYISSGSSYKY | ARDPVVTEYYYYGMD | NSWLAWY | LLIYAASSLQ | QQANSFPW |
| | | 1141 | 1142 | 1143 | 1144 | 1145 | 1146 |
| 35 | VG9B88 | SSYTMN | WVSSISTSSSYID | ARDGDMVAPIKGSFD | LNSDDGNTYLDWY | LLIYTLSYRA | MQRIEFPI |
| | | 1175 | 1176 | 1177 | 1178 | 1179 | 1180 |
| 36 | VG9B384 | SSSGMCVS | WLTLIDWFDDKY | ARIRGTGAYYYGLD | DKYASWY | LVIYQDNKRP | QAWDSSKV |
| | | 1209 | 1210 | 1211 | 1212 | 1213 | 1214 |
| 37 | VG9B413 | SNYDMN | WVSSISSSSSYIY | ARDVGVTTDYYYYGMD | ASSYLAWY | LLIYGASSRA | QQYGSSPPY |
| | | 1243 | 1244 | 1245 | 1246 | 1247 | 1248 |
| 38 | VG9B36 | SSYGMY | WVSSISTGSSYIY | ARDKGLAVTGYIMD | SNYLNWY | LLIYDASNLE | QQYDNLPMY |
| | | 1277 | 1278 | 1279 | 1280 | 1281 | 1282 |
| 39 | VG9B403 | SNYDMN | WVSSISSSSSYIY | ARDVGVTTDYYYYGMD | ASSYLAWY | LLIYGASSRA | QQYGSSPPY |
| | | 1311 | 1312 | 1313 | 1314 | 1315 | 1316 |
| 40 | VG9B191 | SRYSMN | GVSSISSSSSYIY | ARDGPTVNWDYYFD | IGNNYVSWY | LLIYDNNKRP | GTWDSSLSTSV |
| | | 1345 | 1346 | 1347 | 1348 | 1349 | 1350 |
| 41 | VG9B44 | SSNSAAWN | WLGRTYYRSKWYND | ARESGSYYTDGFD | SSYLAWY | LLIYAASTLQ | QEFNSYPY |
| | | 1379 | 1380 | 1381 | 1382 | 1383 | 1384 |
| 42 | VG9B67 | SSYTMN | WVSSISSSSSYID | ARDGDILATIRGSFD | LDSDAGNTYLDWY | LLIYTLSYRA | MQRIEFPI |
| | | 1413 | 1414 | 1415 | 1416 | 1417 | 1418 |
| 43 | VG9B402 | SNYGMN | WVSYISSGSSYKY | ARDPVVTEYYYYGMD | NSWLAWY | LLIYAASSLQ | QQANSFPW |
| | | 1447 | 1448 | 1449 | 1450 | 1451 | 1452 |
| 44 | VG9B127 | GAYTMN | WVSSISSSSSYID | ARDGDIVSTIRGSFD | LDSDDGNTYLDWY | LLIYTLSYRA | MQRIEFPI |
| | | 1481 | 1482 | 1483 | 1484 | 1485 | 1486 |
| 45 | VG9B137 | SSYSMD | WVSSIGSSSSYIF | ATSYSWNYGGAFD | SSSYLAWY | LLIYGASSRA | QQYGSSPPY |
| | | 1515 | 1516 | 1517 | 1518 | 1519 | 1520 |
| 46 | VG9B33 | SSYNMN | WVSSISTSSSYIY | ARDTSVTKYPDTFD | NNYLAWF | LLIYGASTLQ | QKYNSAPF |
| | | 1549 | 1550 | 1551 | 1552 | 1553 | 1554 |
| 47 | VG9B162 | TGDYMH | WMGWINPNSGYTN | AREGDAFD | VGGYNYVSWY | LMIYEVSKRP | NSYAGSNNFE |
| | | 1583 | 1584 | 1585 | 1586 | 1587 | 1588 |
| 48 | VG9B152 | SSYSMI | WVSSISSSSDYIY | ARDWELLGFD | DKYACWY | LVIYQHNKRP | QAWDSTTV |
| | | 1617 | 1618 | 1619 | 1620 | 1621 | 1622 |
| 49 | VG9B64 | SISGVSVG | WLALIYWNDDKR | VHSGQWLEGDAFD | VHSDGNTYLSWL | LLIYKISNRF | MQATQFPL |
| | | 1651 | 1652 | 1653 | 1654 | 1655 | 1656 |
| 50 | VG9B21 | SNYDMN | WVSSISSSSHYIY | ARDRGVTTDYYYYALD | YNYLAWY | LLIYAASTLQ | QKYNRAPF |
| | | 1685 | 1686 | 1687 | 1688 | 1689 | 1690 |
| 51 | VG9B128 | GTYGMH | WVAVIWYNGSNKY | ARGGFGESFD | IDYLAWF | LLIYDASNRA | QQRSNWPL |
| | | 1719 | 1720 | 1721 | 1722 | 1723 | 1724 |
| 52 | VG9B66 | STYTMN | WVSSISSSSFYMD | ARDGDIVATIRGSFD | TNFLAWY | LLIYTASTLQ | QKYNSAPF |
| | | 1753 | 1754 | 1755 | 1756 | 1757 | 1758 |
| 53 | VG9B32 | SRYAMN | WVSFISGTGYTVY | ARDQEPGFD | VHSDGNTYLSWL | LLIYKISNRF | MQATHFPF |
| | | 1787 | 1788 | 1789 | 1790 | 1791 | 1792 |
| 54 | VG9B57 | SSHDMN | WVSSISSSSSYIF | ARDLGVGVRDYYYYGMD | TNYLAWF | RLIYAASSLQ | LQHDTYPY |
| | | 1821 | 1822 | 1823 | 1824 | 1825 | 1826 |
| 55 | VG9B135 | TTYGVGVG | WLALIYWNDDKR | AHDYDFWSGYFD | SSSYLAWY | LLIYGASSRA | QQYGSSPL |
| | | 1855 | 1856 | 1857 | 1858 | 1859 | 1860 |
| 56 | VG9B60 | TGYYMH | WMGRINPNSGVTH | ARGGSLVRGVISGLD | SGSYLAWY | LLIYGASSRA | QQYGSSPPY |
| | | 1889 | 1890 | 1891 | 1892 | 1893 | 1894 |
| 57 | VG9B409 | TSYYMH | WMGIINPSGGSTS | ARGSYGWYFD | TSSYLAWY | LLIYGASSRA | QQYGSSPPY |
| | | 1923 | 1924 | 1925 | 1926 | 1927 | 1928 |
| 58 | VG9B411 | SNYDMN | WVSSISSSSSYIY | ARDVGVTTDYYYYGMD | NSWLAWY | LLIYAASTLQ | QQYNNWPR |
| | | 1957 | 1958 | 1959 | 1960 | 1961 | 1962 |
| 59 | VG9B129 | SSYCIS | WMGGILPILSTAN | ARAHDYYYGMD | SNNYLAWY | LLIYGASSRA | QQYGSSPPY |
| | | 1991 | 1992 | 1993 | 1994 | 1995 | 1996 |
| 60 | VG9B396 | RGYYWN | WIGRIFTTGNTN | AREKWDSSSSALYFD | IGADYDVKWY | LLIYGNTDRP | QSYDSRLTGYV |
| | | 2025 | 2026 | 2027 | 2028 | 2029 | 2030 |
| 61 | VG9B470 | TTSGVGVG | WLALILWNDHTI | ARDKWELRDAFD | SRYLAWY | LLIYVASTLQ | QQLISYPY |
| | | 2059 | 2060 | 2061 | 2062 | 2063 | 2064 |
| 62 | VG9B111 | STYSVN | WVSSISSDSSYIF | ARDSVTGPFD | SHYLNWY | LLIYDSYILE | QQYDNLPY |
| | | 2093 | 2094 | 2095 | 2096 | 2097 | 2098 |
| 63 | VG9B169 | TNYFWN | WIGYIFYSGSTS | ARVGRWELRTAFD | IGSNTVNWY | LLIYSNNQRP | AAWDDSLNGPG |
| | | 2127 | 2128 | 2129 | 2130 | 2131 | 2132 |
| 64 | VG9B639 | TTYRMN | WVSSISSSSIYIH | ARERVYTVSFD | SHYLNWY | LLIYDSYILE | QQYDNLPY |
| | | 2161 | 2162 | 2163 | 2164 | 2165 | 2166 |
| 65 | VG9B201 | TTHGVGVG | WLALIYWNADKH | AHEGDWGHYFD | IGNNDVSWY | LLIYDNNRRP | ETWDSSLSAIW |
| | | 2195 | 2196 | 2197 | 2198 | 2199 | 2200 |
| 66 | VG9B161 | SSYFWS | WIGYIFYSGSTN | ARVGRWELRGAFD | IGSNTVNWY | LLIYSNNQRP | AAWDDSLNSPG |
| | | 2229 | 2230 | 2231 | 2232 | 2233 | 2234 |
| 67 | VG9B383 | SSYYWN | WVGRIYTIGNTN | AREGYYDSSGSFFPGAFG | VGSYNLVSWY | LMIYAGSKRP | CSFAGATNV |
| | | 2263 | 2264 | 2265 | 2266 | 2267 | 2268 |
| 68 | VG9B382 | SGYYWN | WIGRIFTTGNTN | ARERWDSSSSALYFD | IGADYDIKWY | LLIYGNSNRP | QSYDSSMSGYV |
| | | 2297 | 2298 | 2299 | 2300 | 2301 | 2302 |
| 69 | VG9B156 | SGYFWN | WIGYIFYSGSTN | AKLGKWELRTAFD | IGSDTVNWY | LLIYSNNQRP | AAWDDSLNGP |
| | | 2331 | 2332 | 2333 | 2334 | 2335 | 2336 |
| 70 | VG9B205 | SGYFWN | WIGYIFYSGSTN | AKLGKWELRTAFD | DKYACWY | LVIYQDSKRP | QAWDSSTV |
| | | 2365 | 2366 | 2367 | 2368 | 2369 | 2370 |
| 71 | VG9B86 | SSSYWS | CIGRFYSSGSTS | ARYSGSYWYFD | SSYLAWY | LLIYDTSNRA | QQRSDWLL |
| | | 2399 | 2400 | 2401 | 2402 | 2403 | 2404 |
| 72 | VG9B154 | SYYFWN | WIGYIYYSGSTN | AREGKWELRTTFD | IGSNTVNWY | LLIYSNNQRP | AAWDDSLNGP |
| | | 2433 | 2434 | 2435 | 2436 | 2437 | 2438 |
| 73 | VG9B159 | SSYYWS | WIGRFYTGGRNN | ARDMEYYYDRSGYSYWYFD | SKSVHWY | LVVYDDSDRP | QVWDSSSDHV |
| | | 2467 | 2468 | 2469 | 2470 | 2471 | 2472 |
| 74 | VG9B465 | TSYYWN | WIGRIYTIGNTN | AREGYYESDGSFFPGAFN | VGSYNLVSWY | LMIYAGSKRP | CSYAGTTNV |
| | | 2501 | 2502 | 2503 | 2504 | 2505 | 2506 |
| 75 | VG9B194 | TGDYMH | WMGWINPNSGYTN | TRELDALD | VGGYNYVSWY | LMIYEVSKRP | NSYAGSNNFE |
| | | 2535 | 2536 | 2537 | 2538 | 2539 | 2540 |
| 76 | VG9B182 | NNHFWS | WIGFVFYNGNTN | ARVGRWVLRTAFD | IGSNTVNWY | LLIYSNNQRP | AAWDDSLNGPG |
| | | 2569 | 2570 | 2571 | 2572 | 2573 | 2574 |
| 77 | VG9B173 | NNYFWS | WIGYIYYSGSTN | AREGKWELRSAFD | IESNTVNWY | LLIYSNNQRP | TAWDDSLNGP |
| | | 2603 | 2604 | 2605 | 2606 | 2607 | 2608 |
| 78 | VG9B87 | TSEYIH | WMGIINPSGGSTS | ARERGYSYGSFD | SSYLAWY | LLIYAASTLQ | QQFNSYSL |
| | | 2637 | 2638 | 2639 | 2640 | 2641 | 2642 |
| 79 | VG9B208 | SGYYWS | LIGYIFYSGSIN | ARVGKWELRSSFD | IGNNDVSWY | LLIYDNNKRP | GTWDSSLSVW |
| | | 2671 | 2672 | 2673 | 2674 | 2675 | 2676 |
| 80 | VG9B372 | SNGGFYWS | WLGYINYSGSTY | ARDRNYEWNFD | SSYLAWY | LLVYDASNRA | QQRSNWPL |
| | | 2705 | 2706 | 2707 | 2708 | 2709 | 2710 |
| 81 | VG9B186 | TNIRMSVS | WLAHIFSNDEKS | ARMRLPYGMD | IGSNTVNWY | LLIYSNNQRP | AAWDDSLNGP |
| | | 2739 | 2740 | 2741 | 2742 | 2743 | 2744 |
| 82 | VG9B177 | NNYFWS | WIGYIFYSGSTN | ARVGKWELRTAFD | VADYNYVSWY | LMIYEVSNRP | CSYTSSFTV |
| | | 2773 | 2774 | 2775 | 2776 | 2777 | 2778 |
| 83 | VG9B114 | SRYDMH | WVSAIGSAGDTY | ARGKWELRDAFD | HSYLAWY | LLIYVASTLQ | QQLNSYPY |
| | | 2807 | 2808 | 2809 | 2810 | 2811 | 2812 |
| 84 | VG9B147 | SGYFWN | WIGYIFYSGSTN | AREGKWELRSTFD | SYYATWY | LVIYGENNRP | NSRDTGDHHL |
| | | 2841 | 2842 | 2843 | 2844 | 2845 | 2846 |
| 85 | VG9B65 | NIYAIN | WMGGIIPFFGTAN | ARGGDSGYDWGFD | VHSDGNTYLSWL | LLIYQISNRF | MQATQFPL |
| | | 2875 | 2876 | 2877 | 2878 | 2879 | 2880 |
| 86 | VG9B81 | NTYYWS | LIGRIYTSDNTN | ARYNWNYWYFD | SSYLAWY | LLIYTASTLQ | QHLNSYPY |
| | | 2909 | 2910 | 2911 | 2912 | 2913 | 2914 |
| 87 | VG9B203 | GHYYWN | WIGRIYTSGSTN | ARSGGNFYWYFD | IGNNYVSWY | LLIYDNNKRP | GTWDSSLSAG |
| | | 2943 | 2944 | 2945 | 2946 | 2947 | 2948 |
| 88 | VG9B380 | SNYYWS | WIGRIYPSGITS | AGIMGTKGAFD | VGGYNYVSWY | LLIYEVSNRP | SSYTSTSV |
| | | 2977 | 2978 | 2979 | 2980 | 2981 | 2982 |
| 89 | VG9B103 | SSYAIS | WMGGIIPIFGTAT | ARGVGWGTDYYYGLD | VHSDGNTYLSWL | LLIYKISNRF | MQATHHPL |
| | | 3011 | 3012 | 3013 | 3014 | 3015 | 3016 |
| 90 | VG9B462 | SNYYWS | WIGRIYSSGITN | AGIVGVKGAFA | VGGYNYVSWY | LMIYEVSNRP | SSYTSTSV |
| | | 3045 | 3046 | 3047 | 3048 | 3049 | 3050 |
| 91 | VG9B461 | SNYYWS | WIGRIYSSGITN | AGIVGVKGAFA | VGGYNYVSWY | LMIYEVSNRP | SSYTSTSV |
| | | 3079 | 3080 | 3081 | 3082 | 3083 | 3084 |
| 92 | VG9B106 | TGYYLH | WVGWINPSSGDTD | ANELGIGVFD | LYSSNNKNYLGWY | LLIYWASTRE | QQYYSIPY |
| | | 3113 | 3114 | 3115 | 3116 | 3117 | 3118 |
| 93 | VG9B115 | STYGVGVG | WLALIYWNDDKR | SHESDWSYYFD | SSYLAWY | LLIYDASNRA | QQRSSWPW |
| | | 3147 | 3148 | 3149 | 3150 | 3151 | 3152 |
| 94 | VG9B27 | SSYVIS | WMGGILPILSTAN | ARAHDYYYGMD | VHSDGNTYLSWL | LLVYKISNRF | MQATHHPL |
| | | 3181 | 3182 | 3183 | 3184 | 3185 | 3186 |
| 95 | VG9B458 | SNYYWS | WIGRIYSSGITN | AGIVGVKGAFA | VGGYNYVSWY | LMIYEVSNRP | SSYTSTSV |
| | | 3215 | 3216 | 3217 | 3218 | 3219 | 3220 |
| 96 | VG9B131 | SSYDMN | WVSYISSSSTAKY | AREDIVVVTPILQ | SNYLAWY | LLIYDASNRA | QQRSNW |
| | | 3249 | 3250 | 3251 | 3252 | 3253 | 3254 |
| 97 | VG9B163 | SNHGMS | WVASITRGGDTTY | TRGPLTVGYAMD | IGHNYVSWY | LLIYDNNQRP | GIWDSSLSIV |
| | | 3283 | 3284 | 3285 | 3286 | 3287 | 3288 |
| 98 | VG9B454 | SNYYWS | WIGRIYSSGITN | AGIVGVKGAFA | VGGYNYVSWY | LMIYEVSNRP | SSYTSTSV |
| | | 3317 | 3318 | 3319 | 3320 | 3321 | 3322 |
| 99 | VG9B439 | SNYYWS | WIGRIYSSGITN | AGIVGVKGAFA | VGGYNYVSWY | LMIYEVSNRP | SSYTSTSV |
| | | 3351 | 3352 | 3353 | 3354 | 3355 | 3356 |
| 100 | VG9B68 | STYAIS | WMGGIIPIFGTAT | ARGVGWGSDYYYGLD | VHSDGNTYLSWL | LLIYKISNRF | IQATHHPL |
| | | 3385 | 3386 | 3387 | 3388 | 3389 | 3390 |
| 101 | VG9B449 | SNYYWS | WIGRIYSSGITN | AGIVGVKGAFA | VGGYNYVSWY | LMIYEVSNRP | SSYTSTSV |
| | | 3419 | 3420 | 3421 | 3422 | 3423 | 3424 |
| 102 | VG9B204 | SSFYWS | WIGRIYTSGGTI | ARWLRAFD | DKYACWY | LVIYQDSKRP | QAWDSSTV |
| | | 3453 | 3454 | 3455 | 3456 | 3457 | 3458 |
| 103 | VG9B459 | SNYYWS | WIGRIYSSGITN | AGIVGVKGAFA | VGGYNYVSWY | LMIYEVSNRP | SSYTSTSV |
| | | 3487 | 3488 | 3489 | 3490 | 3491 | 3492 |
| 104 | VG9B157 | SNHGMS | WVASITRGGDTTY | TRGPLTVGYAMD | SYYATWY | LVIYGENNRP | NSRDTGDHHL |
| | | 3521 | 3522 | 3523 | 3524 | 3525 | 3526 |
| 105 | VG9B453 | SNYYWS | WIGRIYSSGITN | AGIVGVKGAFA | VGGYNYVSWY | LMIYEVSNRP | SSYTSTSV |
| | | 3555 | 3556 | 3557 | 3558 | 3559 | 3560 |
| 106 | VG9B443 | SNYYWS | WIGRIYSSGITN | AGIVGVKGAFA | VGGYNYVSWY | LMIYEVSNRP | SSYTSTSV |
| | | 3589 | 3590 | 3591 | 3592 | 3593 | 3594 |
| 107 | VG9B455 | SNYYWS | WIGRIYSSGITN | AGIVGVKGAFA | VGGYNYVSWY | LMIYEVSNRP | SSYTSTSV |
| | | 3623 | 3624 | 3625 | 3626 | 3627 | 3628 |
| 108 | VG9B466 | SNYYWS | WIGRIYSSGITN | AGIVGVKGAFA | VGGYNYVSWY | LMIYEVSNRP | SSYTSTSV |
| | | 3657 | 3658 | 3659 | 3660 | 3661 | 3662 |
| 109 | VG9B450 | SNYYWS | WIGRIYSSGITN | AGIVGVKGAFA | VGGYNYVSWY | LMIYEVSNRP | SSYTSTSV |
| | | 3691 | 3692 | 3693 | 3694 | 3695 | 3696 |
| 110 | VG9B438 | SNYYWS | WIGRIYSSGITN | AGIVGVKGAFA | VGGYNYVSWY | LMIYEVSNRP | SSYTSTSV |
| | | 3725 | 3726 | 3727 | 3728 | 3729 | 3730 |
| 111 | VG9B464 | SNYYWS | WIGRIYSSGITN | AGIVGVKGAFA | VGGYNYVSWY | LMIYEVSNRP | SSYTSTSV |
| | | 3759 | 3760 | 3761 | 3762 | 3763 | 3764 |
| 112 | VG9B437 | SNYYWS | WIGRIYSSGITN | AGIVGVKGAFA | VGGYNYVSWY | LMIYEVSNRP | SSYTSTSV |
| | | 3793 | 3794 | 3795 | 3796 | 3797 | 3798 |
| 113 | VG9B457 | SNYYWS | WIGRIYSSGITN | AGIVGVKGAFA | VGGYNYVSWY | LMIYEVSNRP | SSYTSTSV |
| | | 3827 | 3828 | 3829 | 3830 | 3831 | 3832 |
| 114 | VG9B442 | SNYYWS | WIGRIYSSGITN | AGIVGVKGAFA | VGGYNYVSWY | LMIYEVSNRP | SSYTSTSV |
| | | 3861 | 3862 | 3863 | 3864 | 3865 | 3866 |
| 115 | VG9B436 | SNYYWS | WIGRIYSSGITN | AGIVGVKGAFA | VGGYNYVSWY | LMIYEVSNRP | SSYTSTSV |
| | | 3895 | 3896 | 3897 | 3898 | 3899 | 3900 |
| 116 | VG9B434 | SNYYWS | WIGRIYSSGITN | AGIVGVKGAFA | VGGYNYVSWY | LMIYEVSNRP | SSYTSTSV |
| | | 3929 | 3930 | 3931 | 3932 | 3933 | 3934 |
| 117 | VG9B460 | SNYYWS | WIGRIYSSGITN | AGIVGVKGAFA | VGGYNYVSWY | LMIYEVSNRP | SSYTSTSV |
| | | 3963 | 3964 | 3965 | 3966 | 3967 | 3968 |
| 118 | VG9B189 | SNHGMS | WVASITRGGDTTY | TRGPLTVGYAMD | VGGYNYVSWY | LMIYEVSKRP | NSYAGSNNFE |
| | | 3997 | 3998 | 3999 | 4000 | 4001 | 4002 |
| 119 | VG9B452 | SNYYWS | WIGRIYSSGITN | AGIVGVKGAFA | VGGYNYVSWY | LMIYEVSNRP | SSYTSTSV |
| | | 4031 | 4032 | 4033 | 4034 | 4035 | 4036 |
| 120 | VG9B53 | SSYYWS | WIGRIHTIGSIN | AMEGVGATNYYYGMA | LHSNGYNYLDWY | LLIYLGSNRA | MQALQTPY |
| | | 4065 | 4066 | 4067 | 4068 | 4069 | 4070 |
| 121 | VG9B41 | TTYWMH | WIGLIDPSDSETR | ASNRWLL | VHSDGNTYLSWL | LLIYKISNRF | VQTREFPL |
| | | 4099 | 4100 | 4101 | 4102 | 4103 | 4104 |
| 122 | VG9B136 | SSNRAAWN | WLGRTYYRSKWYNE | ARDLWELREACD | SSYLAWY | LLIYVASTLQ | QQLNSYPF |
| | | 4133 | 4134 | 4135 | 4136 | 4137 | 4138 |
| 123 | VG9B399 | SNYDMN | WVSSISSSSSYIY | ARDVGVTTDYYYYGMD | ASSYLAWY | LLIYGASSRA | QQYGSSPPY |
| | | 4167 | 4168 | 4169 | 4170 | 4171 | 4172 |
| 124 | VG9B369 | STIGVGVG | WLSLIYWNDDKR | AHSHDWVHAFD | SHYLNWY | FLIYDASNLD | QQYDNFPL |
| | | 4201 | 4202 | 4203 | 4204 | 4205 | 4206 |
| 125 | VG9B144 | TGYYMH | WMGWINPNSGATN | AREDDAFD | NYYATWF | LVFFGKNNRP | NSRDSSGNHV |
| | | 4235 | 4236 | 4237 | 4238 | 4239 | 4240 |
| 126 | VG9B410 | SNYGMN | WVSYISSGSSYKY | ARDPVVTEYYYYGMD | NSWLAWY | LLIYAASSLQ | QQANSFPW |
| | | 4269 | 4270 | 4271 | 4272 | 4273 | 4274 |
| 127 | VG9B386 | SSYSMI | WVSSIGSSSTYIY | ARDGETGGFD | SRSYLAWY | LLIYGPSNRA | QQYGSLPL |
| | | 4303 | 4304 | 4305 | 4306 | 4307 | 4308 |
| 128 | VG9B125 | SSYDMN | WVSYISSSSTAKY | AREDIVVVTPILQ | SKDLAWY | LLIYDASNRA | QQRINWPLF |
| | | 4337 | 4338 | 4339 | 4340 | 4341 | 4342 |
| 129 | VG9B112 | SSNSAAWN | WLGGTYYRSKWFNN | ARGEWGLRDAFD | HSYLAWY | LLIYVASTLQ | QQLNSYPW |
| | | 4371 | 4372 | 4373 | 4374 | 4375 | 4376 |
| 130 | VG9B371 | SSYDMN | WVSSISSSSSYIY | ARDRGVGGTDYYYYGLD | SSSYLAWY | LLIYGASSRA | QQYGSSPPY |
| | | 4405 | 4406 | 4407 | 4408 | 4409 | 4410 |
| 131 | VG9B387 | TGYYLQ | WMGWINPNSGYTD | ARLDDAFD | NYYAIWY | LVIFGKNNRP | NSRDSSGNHW |
| | | 4439 | 4440 | 4441 | 4442 | 4443 | 4444 |
| 132 | VG9B47 | SNYAIS | WMGGIIPFFGTPD | STGGGYGDYDYYYGIN | VHSDGNTYLSWL | LLIYKISNRF | MQAKEFPL |
| | | 4473 | 4474 | 4475 | 4476 | 4477 | 4478 |
| 133 | VG9B379 | TGDYIH | WMGWINPNSGGTN | AREGGVAPAAPDAFD | SYYARWY | VVIYGKNNRP | NSRDSSGNHR |
| | | 4507 | 4508 | 4509 | 4510 | 4511 | 4512 |
| 134 | VG9B447 | SNYYWS | WIGRIYSSGITN | AGIVGVKGAFA | VGGYNYVSWY | LMIYEVSNRP | SSYTSTSV |
| | | 4541 | 4542 | 4543 | 4544 | 4545 | 4546 |
| 135 | VG9B392 | TGYYIH | WMGWINPNSGYTN | AREDDAFD | SYYARWY | LVIYGKNNRP | NSRDNSGNHV |
| | | 4575 | 4576 | 4577 | 4578 | 4579 | 4580 |
| 136 | VG9B96 | SNYAIS | WMGGIIPIFSAGT | SSNSGTYYDYYYGMD | VHSDGNTYLSWL | LLIYKIFNRL | MQAKQFPL |
| | | 4609 | 4610 | 4611 | 4612 | 4613 | 4614 |
| 137 | VG9B77 | SSYAIS | WMGGIIPFFGTAD | ATATVTTDYYYGMD | VHSDGNTYLSWL | LLIYEISNRF | MQARQFPL |
| | | 4643 | 4644 | 4645 | 4646 | 4647 | 4648 |

TABLE 7
IMGT CDR Amino Acid Sequences (SEQ ID NO
disclosed in the cell under each respective sequence)

| | Protein | HC IMGT | HC IMGT | HC IMGT | LC IMGT | LC IMGT | LC IMGT |
|---|---|---|---|---|---|---|---|
| # | Name | CDR1 | CDR2 | CDR3 | CDR1 | CDR2 | CDR3 |
| 1 | VG9B54 | GFAFSSYS | ISSSSSYI | ATDGELGPFDY | QSVSSSY | GAS | QQYGRSPLT |
| | | 25 | 26 | 27 | 28 | | 30 |
| 2 | VG9B121 | GFTFSDYA | ISSTSNYI | ANSYNWNYGGAFDI | QSVSSSY | GAS | QQYGRSPLT |
| | | 59 | 60 | 61 | 62 | | 64 |
| 3 | VG9B429 | GFTFSNYD | ISSSSSYI | ARDVGVTTDYYYYGMDV | QGINSW | AAS | QQANSFPWT |
| | | 93 | 94 | 95 | 96 | | 98 |
| 4 | VG9B370 | RFTLSSYD | ISSSSSYI | ARDRGVGGTDYYYYGLDV | QSVSSSY | GAS | QQYGSSPPYT |
| | | 127 | 128 | 129 | 130 | | 132 |
| 5 | VG9B80 | GFTFSTYS | ISSSSSYI | AKDGELGVFDY | QSISSSY | GPS | QQFGRSPLT |
| | | 161 | 162 | 163 | 164 | | 166 |
| 6 | VG9B414 | GFTFSNYD | ISSSSSYI | ARDVGVTTDYYYYGMDV | QGINSW | AAS | QQANSFPWT |
| | | 195 | 196 | 197 | 198 | | 200 |
| 7 | VG9B195 | GFTFSPYT | ISSSSSYM | ARDGDLVGPTYYFDY | KLGDKY | QHN | QAWDSTTVV |
| | | 229 | 230 | 231 | 232 | | 234 |
| 8 | VG9B140 | GFTFRSYD | ISTSSGYI | ARDRGIAVAGDYYYGMDV | QSVSSSY | GAS | QQYGSSPPYT |
| | | 263 | 264 | 265 | 266 | | 268 |
| 9 | VG9B426 | GFTFSNYD | ISSSSSYI | ARDVGVTTDYYYYGMDV | QSVASSY | GAS | QQYGSSPPYT |
| | | 297 | 298 | 299 | 300 | | 302 |
| 10 | VG9B46 | GFTFSINS | ISSTSDYI | VRDDVFGAFDI | QSVSRSY | GSS | QQYSRSPLT |
| | | 331 | 332 | 333 | 334 | | 336 |
| 11 | VG9B416 | GFTFSNYD | ISSSSSYI | ARDVGVTTDYYYYGMDV | QSVASSY | GAS | QQYGSSPPYT |
| | | 365 | 366 | 367 | 368 | | 370 |
| 12 | VG9B69 | GFTFSSYD | ITSSSYYI | ARDLGVRGVDYYYYGLDV | QSVSSSY | GAS | QQYGSSPPYT |
| | | 399 | 400 | 401 | 402 | | 404 |
| 13 | VG9B415 | GFTFSNYD | ISSSSSYI | ARDVGVTTDYYYYGMDV | QSVASSY | GAS | QQYGSSPPYT |
| | | 433 | 434 | 435 | 436 | | 438 |
| 14 | VG9B104 | GFTFSVYS | IGSSSSYI | GRDHDYGGLDY | QSVSSSY | GPS | QQYGRSPLT |
| | | 467 | 468 | 469 | 470 | | 472 |
| 15 | VG9B198 | GDSISSIY | IYTTDIT | AKNGYSYGGFNY | SSNIGAGYD | GDS | QSYDSSLSVVV |
| | | 501 | 502 | 503 | 504 | | 506 |
| 16 | VG9B463 | GFTFSNYD | ISSSSSYI | ARDVGVTTDYYYYGMDV | SSNIGSNT | TNT | AAWDDSLNAWV |
| | | 535 | 536 | 537 | 538 | | 540 |
| 17 | VG9B469 | GFTFSHYD | ISSSSSYI | ARDRGVGDTSDYYSFGLDV | QNVSSTY | GAC | QQYGSSPPYT |
| | | 569 | 570 | 571 | 572 | | 574 |
| 18 | VG9B428 | GFTFSNYG | ISSGSSYK | ARDPVVTEYYYYGMDV | QGINSW | AAS | QQANSFPWT |
| | | 603 | 604 | 605 | 606 | | 608 |
| 19 | VG9B430 | GFTFSSYD | ISSSSSYI | ARDVGVTTDYYYYGMDV | QSVASSY | GAS | QQYGSSPPYT |
| | | 637 | 638 | 639 | 640 | | 642 |
| 20 | VG9B423 | GFTFSNYD | ISSSSSYI | ARDVGVTTDYYYYGMDV | QSVASSY | GAS | QQYGSSPPYT |
| | | 671 | 672 | 673 | 674 | | 676 |
| 21 | VG9B98 | GFTFSSYS | IGSSSTYI | ARDGELGPFEY | QSVSTSY | GTS | LQYGRSPLT |
| | | 705 | 706 | 707 | 708 | | 710 |
| 22 | VG9B73 | GFTFSNYD | ISSSSSYI | AREIGVTGTTYYQDYGMDV | QSFSSNY | GAS | QQYGSSPPFT |
| | | 739 | 740 | 741 | 742 | | 744 |
| 23 | VG9B133 | GFTFSNYD | ISSSSSYI | ARDLGITGTTMDYYYGMDV | QSVSSSY | GAS | QQYGSSPPYT |
| | | 773 | 774 | 775 | 776 | | 778 |
| 24 | VG9B368 | GFTFNSYD | ISSSSSYI | ARDRGIGGDYYSYAMDV | QSVSSSY | GAS | QQYGSSPPYT |
| | | 807 | 808 | 809 | 810 | | 812 |
| 25 | VG9B424 | GFTFSNYD | ISSSSSYI | ARDVGVTTDYYYYGMDV | QSVASSY | GAS | QQYGSSPPYT |
| | | 841 | 842 | 843 | 844 | | 846 |
| 26 | VG9B427 | GFTFSNYD | ISSSSSYI | ARDVGVTTDYYYYGMDV | QSVASSY | GAS | QQYGSSPPYT |
| | | 875 | 876 | 877 | 878 | | 880 |
| 27 | VG9B417 | GFTFSNYG | ISSGSSYK | ARDPVVTEYYYYGMDV | QGINSW | AAS | QQANSFPWT |
| | | 909 | 910 | 911 | 912 | | 914 |
| 28 | VG9B58 | GGSIKSSY | MFYLGST | SRERPVLDAFDI | QDITNY | AAS | LQHDTYPYT |
| | | 943 | 944 | 945 | 946 | | 948 |
| 29 | VG9B419 | GFTFSNYD | ISSSSSYI | ARDVGVTTDYYYYGMDV | QSVASSY | GAS | QQYGSSPPYT |
| | | 977 | 978 | 979 | 980 | | 982 |
| 30 | VG9B425 | GFTFSNYD | ISSSSSYI | ARDVGVTTDYYYYGMDV | QSVASSY | GAS | QQYGSSPPYT |
| | | 1011 | 1012 | 1013 | 1014 | | 1016 |
| 31 | VG9B143 | GFTFSTYG | ISTSSYTI | AREGDWWYFDL | SSDVGGYNY | EVS | SSYTSSSTLV |
| | | 1045 | 1046 | 1047 | 1048 | | 1050 |
| 32 | VG9B418 | GFTFSNYD | ISSSSSYI | ARDVGVTTDYYYYGMDV | QSVASSY | GAS | QQYGSSPPYT |
| | | 1079 | 1080 | 1081 | 1082 | | 1084 |
| 33 | VG9B472 | GFTFSHYD | ISSSSSYI | ARDRGVGDTSDYYSFGLDV | QGIATY | AAS | QKYNSAPPWT |
| | | 1113 | 1114 | 1115 | 1116 | | 1118 |
| 34 | VG9B421 | GFTFSNYG | ISSGSSYK | ARDPVVTEYYYYGMDV | QGINSW | AAS | QQANSFPWT |
| | | 1147 | 1148 | 1149 | 1150 | | 1152 |
| 35 | VG9B88 | GFTFSSYT | ISTSSSYI | ARDGDMVAPIKGSFDY | QSLLNSDDGNTY | TLS | MQRIEFPIT |
| | | 1181 | 1182 | 1183 | 1184 | | 1186 |
| 36 | VG9B384 | GFSLSSSGMC | IDWFDDK | ARIRGTGAYYYGLDV | ELGDKY | QDN | QAWDSSKVV |
| | | 1215 | 1216 | 1217 | 1218 | | 1220 |
| 37 | VG9B413 | GFTFSNYD | ISSSSSYI | ARDVGVTTDYYYYGMDV | QSVASSY | GAS | QQYGSSPPYT |
| | | 1249 | 1250 | 1251 | 1252 | | 1254 |
| 38 | VG9B36 | GFTFSSYG | ISTGSSYI | ARDKGLAVTGYIMDV | QDISNY | DAS | QQYDNLPMYT |
| | | 1283 | 1284 | 1285 | 1286 | | 1288 |
| 39 | VG9B403 | GFTFSNYD | ISSSSSYI | ARDVGVTTDYYYYGMDV | QSVASSY | GAS | QQYGSSPPYT |
| | | 1317 | 1318 | 1319 | 1320 | | 1322 |
| 40 | VG9B191 | GFTFSRYS | ISSSSSYI | ARDGPTVNWDYYFDL | SSNIGNNY | DNN | GTWDSSLSTSVV |
| | | 1351 | 1352 | 1353 | 1354 | | 1356 |
| 41 | VG9B44 | GDSVSSNSAA | TYYRSKWYN | ARESGSYYTDGFDI | QGISSY | AAS | QEFNSYPYT |
| | | 1385 | 1386 | 1387 | 1388 | | 1390 |
| 42 | VG9B67 | GFTFSSYT | ISSSSSYI | ARDGDILATIRGSFDY | QSLLDSDAGNTY | TLS | MQRIEFPIT |
| | | 1419 | 1420 | 1421 | 1422 | | 1424 |
| 43 | VG9B402 | GFTFSNYG | ISSGSSYK | ARDPVVTEYYYYGMDV | QGINSW | AAS | QQANSFPWT |
| | | 1453 | 1454 | 1455 | 1456 | | 1458 |
| 44 | VG9B127 | GFTFGAYT | ISSSSSYI | ARDGDIVSTIRGSFDY | QSLLDSDDGNTY | TLS | MQRIEFPIT |
| | | 1487 | 1488 | 1489 | 1490 | | 1492 |
| 45 | VG9B137 | GITFSSYS | IGSSSSYI | ATSYSWNYGGAFDI | QSVSSSY | GAS | QQYGSSPPYT |
| | | 1521 | 1522 | 1523 | 1524 | | 1526 |
| 46 | VG9B33 | GFTFSSYN | ISTSSSYI | ARDTSVTKYPDTFDI | QDINNY | GAS | QKYNSAPFT |
| | | 1555 | 1556 | 1557 | 1558 | | 1560 |
| 47 | VG9B162 | GYTFTGDY | INPNSGYT | AREGDAFDV | SSDVGGYNY | EVS | NSYAGSNNFEV |
| | | 1589 | 1590 | 1591 | 1592 | | 1594 |
| 48 | VG9B152 | GFTFSSYS | ISSSSDYI | ARDWELLGFDC | KLGDKY | QHN | QAWDSTTVV |
| | | 1623 | 1624 | 1625 | 1626 | | 1628 |
| 49 | VG9B64 | GFSLSISGVS | IYWNDDK | VHSGQWLEGDAFDI | ESLVHSDGNTY | KIS | MQATQFPLT |
| | | 1657 | 1658 | 1659 | 1660 | | 1662 |
| 50 | VG9B21 | GFSFSNYD | ISSSSHYI | ARDRGVTTDYYYYALDV | QGIYNY | AAS | QKYNRAPFT |
| | | 1691 | 1692 | 1693 | 1694 | | 1696 |
| 51 | VG9B128 | GFTFGTYG | IWYNGSNK | ARGGFGESFDS | QSVIDY | DAS | QQRSNWPLT |
| | | 1725 | 1726 | 1727 | 1728 | | 1730 |
| 52 | VG9B66 | GFTFSTYT | ISSSSFYM | ARDGDIVATIRGSFDY | QDITNF | TAS | QKYNSAPFT |
| | | 1759 | 1760 | 1761 | 1762 | | 1764 |
| 53 | VG9B32 | GFTFSRYA | ISGTGYTV | ARDQEPGFDY | QSLVHSDGNTY | KIS | MQATHFPFT |
| | | 1793 | 1794 | 1795 | 1796 | | 1798 |
| 54 | VG9B57 | GFTFSSHD | ISSSSSYI | ARDLGVGVRDYYYYGMDV | QDITNY | AAS | LQHDTYPYT |
| | | 1827 | 1828 | 1829 | 1830 | | 1832 |
| 55 | VG9B135 | GFSLTTYGVG | IYWNDDK | AHDYDFWSGYFDY | QSVSSSY | GAS | QQYGSSPLT |
| | | 1861 | 1862 | 1863 | 1864 | | 1866 |
| 56 | VG9B60 | GYTFTGYY | INPNSGVT | ARGGSLVRGVISGLDY | QSFSGSY | GAS | QQYGSSPPYT |
| | | 1895 | 1896 | 1897 | 1898 | | 1900 |
| 57 | VG9B409 | GYTLTSYY | INPSGGST | ARGSYGWYFDL | QSVTSSY | GAS | QQYGSSPPYT |
| | | 1929 | 1930 | 1931 | 1932 | | 1934 |
| 58 | VG9B411 | GFTFSNYD | ISSSSSYI | ARDVGVTTDYYYYGMDV | QGINSW | AAS | QQYNNWPRT |
| | | 1963 | 1964 | 1965 | 1966 | | 1968 |
| 59 | VG9B129 | GGTFSSYV | ILPILSTA | ARAHDYYYGMDV | QSVSNNY | GAS | QQYGSSPPYT |
| | | 1997 | 1998 | 1999 | 2000 | | 2002 |
| 60 | VG9B396 | GDSIRGYY | IFTTGNT | AREKWDSSSSALYFDF | SSNIGADYD | GNT | QSYDSRLTGYVV |
| | | 2031 | 2032 | 2033 | 2034 | | 2036 |
| 61 | VG9B470 | GFSLTTSGVG | ILWNDHT | ARDKWELRDAFDI | QGISRY | VAS | QQLISYPYT |
| | | 2065 | 2066 | 2067 | 2068 | | 2070 |
| 62 | VG9B111 | GFTFSTYS | ISSDSSYI | ARDSVTGPFDY | QDISHY | DSY | QQYDNLPYT |
| | | 2099 | 2100 | 2101 | 2102 | | 2104 |
| 63 | VG9B169 | GGSITNYF | IFYSGST | ARVGRWELRTAFDI | SSNIGSNT | SNN | AAWDDSLNGPGV |
| | | 2133 | 2134 | 2135 | 2136 | | 2138 |
| 64 | VG9B639 | GFTFTTYR | ISSSSIYI | ARERVYTVSFDY | QDISHY | DSY | QQYDNLPYT |
| | | 2167 | 2168 | 2169 | 2170 | | 2172 |
| 65 | VG9B201 | GFSLTTHGVG | IYWNADK | AHEGDWGHYFDF | SSNIGNND | DNN | ETWDSSLSAIWV |
| | | 2201 | 2202 | 2203 | 2204 | | 2206 |
| 66 | VG9B161 | GGSISSYF | IFYSGST | ARVGRWELRGAFDI | SSNIGSNT | SNN | AAWDDSLNSPGV |
| | | 2235 | 2236 | 2237 | 2238 | | 2240 |
| 67 | VG9B383 | SGSISSYY | IYTIGNT | AREGYYDSSGSFFPGAFGI | SNDVGSYNL | AGS | CSFAGATNVV |
| | | 2269 | 2270 | 2271 | 2272 | | 2274 |
| 68 | VG9B382 | GDSISGYY | IFTTGNT | ARERWDSSSSALYFDY | SSNIGADYD | GNS | QSYDSSMSGYVV |
| | | 2303 | 2304 | 2305 | 2306 | | 2308 |
| 69 | VG9B156 | NGSISGYF | IFYSGST | AKLGKWELRTAFDI | TSNIGSDT | SNN | AAWDDSLNGPV |
| | | 2337 | 2338 | 2339 | 2340 | | 2342 |
| 70 | VG9B205 | NGSISGYF | IFYSGST | AKLGKWELRTAFDI | KLGDKY | QDS | QAWDSSTVV |
| | | 2371 | 2372 | 2373 | 2374 | | 2376 |
| 71 | VG9B86 | GDSISSSY | FYSSGST | ARYSGSYWYFDL | QSVSSY | DTS | QQRSDWLLT |
| | | 2405 | 2406 | 2407 | 2408 | | 2410 |
| 72 | VG9B154 | GGSISYYF | IYYSGST | AREGKWELRTTFDI | SSNIGSNT | SNN | AAWDDSLNGPV |
| | | 2439 | 2440 | 2441 | 2442 | | 2444 |
| 73 | VG9B159 | GGSISSYY | FYTGGRN | ARDMEYYYDRSGYSYWYFDL | NIGSKS | DDS | QVWDSSSDHVV |
| | | 2473 | 2474 | 2475 | 2476 | | 2478 |
| 74 | VG9B465 | GGSITSYY | IYTIGNT | AREGYYESDGSFFPGAFNI | SNDVGSYNL | AGS | CSYAGTTNVV |
| | | 2507 | 2508 | 2509 | 2510 | | 2512 |
| 75 | VG9B194 | GYTFTGDY | INPNSGYT | TRELDALDV | SSDVGGYNY | EVS | NSYAGSNNFEV |
| | | 2541 | 2542 | 2543 | 2544 | | 2546 |
| 76 | VG9B182 | GDSINNHF | VFYNGNT | ARVGRWVLRTAFDI | SSNIGSNT | SNN | AAWDDSLNGPGV |
| | | 2575 | 2576 | 2577 | 2578 | | 2580 |
| 77 | VG9B173 | GGSINNYF | IYYSGST | AREGKWELRSAFDI | SSNIESNT | SNN | TAWDDSLNGPV |
| | | 2609 | 2610 | 2611 | 2612 | | 2614 |
| 78 | VG9B87 | GYTFTSEY | INPSGGST | ARERGYSYGSFDY | QGISSY | AAS | QQFNSYSLT |
| | | 2643 | 2644 | 2645 | 2646 | | 2648 |
| 79 | VG9B208 | GGSISGYY | IFYSGSI | ARVGKWELRSSFDI | TSNIGNND | DNN | GTWDSSLSVWV |
| | | 2677 | 2678 | 2679 | 2680 | | 2682 |
| 80 | VG9B372 | GGSISNGGFY | INYSGST | ARDRNYEWNFDL | QSVSSY | DAS | QQRSNWPLT |
| | | 2711 | 2712 | 2713 | 2714 | | 2716 |
| 81 | VG9B186 | GFSLTNIRMS | IFSNDEK | ARMRLPYGMDV | TSNIGSNT | SNN | AAWDDSLNGPV |
| | | 2745 | 2746 | 2747 | 2748 | | 2750 |
| 82 | VG9B177 | GGSINNYF | IFYSGST | ARVGKWELRTAFDI | SSDVADYNY | EVS | CSYTSSFTVV |
| | | 2779 | 2780 | 2781 | 2782 | | 2784 |
| 83 | VG9B114 | GFTFSRYD | IGSAGDT | ARGKWELRDAFDI | QGIHSY | VAS | QQLNSYPYT |
| | | 2813 | 2814 | 2815 | 2816 | | 2818 |
| 84 | VG9B147 | GGSISGYF | IFYSGST | AREGKWELRSTFDI | SLRSYY | GEN | NSRDTGDHHLV |
| | | 2847 | 2848 | 2849 | 2850 | | 2852 |
| 85 | VG9B65 | GGTFNIYA | IIPFFGTA | ARGGDSGYDWGFDY | QSLVHSDGNTY | QIS | MQATQFPLT |
| | | 2881 | 2882 | 2883 | 2884 | | 2886 |
| 86 | VG9B81 | GGSINTYY | IYTSDNT | ARYNWNYWYFDL | QGISSY | TAS | QHLNSYPYT |
| | | 2915 | 2916 | 2917 | 2918 | | 2920 |
| 87 | VG9B203 | GDSIGHYY | IYTSGST | ARSGGNFYWYFDL | SSNIGNNY | DNN | GTWDSSLSAGV |
| | | 2949 | 2950 | 2951 | 2952 | | 2954 |
| 88 | VG9B380 | GGSISNYY | IYPSGIT | AGIMGTKGAFDI | SSDVGGYNY | EVS | SSYTSTSVV |
| | | 2983 | 2984 | 2985 | 2986 | | 2988 |
| 89 | VG9B103 | GGTFSSYA | IIPIFGTA | ARGVGWGTDYYYGLDV | QGLVHSDGNTY | KIS | MQATHHPLT |
| | | 3017 | 3018 | 3019 | 3020 | | 3022 |
| 90 | VG9B462 | GGSISNYY | IYSSGIT | AGIVGVKGAFAI | SSDVGGYNY | EVS | SSYTSTSVV |
| | | 3051 | 3052 | 3053 | 3054 | | 3056 |
| 91 | VG9B461 | GGSISNYY | IYSSGIT | AGIVGVKGAFAI | SSDVGGYNY | EVS | SSYTSTSVV |
| | | 3085 | 3086 | 3087 | 3088 | | 3090 |
| 92 | VG9B106 | GYTFTGYY | INPSSGDT | ANELGIGVFDY | QSVLYSSNNKNY | WAS | QQYYSIPYT |
| | | 3119 | 3120 | 3121 | 3122 | | 3124 |
| 93 | VG9B115 | GFSLSTYGVG | IYWNDDK | SHESDWSYYFDY | QSVSSY | DAS | QQRSSWPWT |
| | | 3153 | 3154 | 3155 | 3156 | | 3158 |
| 94 | VG9B27 | GGTFSSYV | ILPILSTA | ARAHDYYYGMDV | QSLVHSDGNTY | KIS | MQATHHPLT |
| | | 3187 | 3188 | 3189 | 3190 | | 3192 |
| 95 | VG9B458 | GGSISNYY | IYSSGIT | AGIVGVKGAFAI | SSDVGGYNY | EVS | SSYTSTSVV |
| | | 3221 | 3222 | 3223 | 3224 | | 3226 |
| 96 | VG9B131 | GFTFSSYD | ISSSSTAK | AREDIVVVTPILQH | QSVSNY | DAS | QQRSNWT |
| | | 3255 | 3256 | 3257 | 3258 | | 3260 |
| 97 | VG9B163 | GFTFSNHG | ITRGGDTT | TRGPLTVGYAMDY | SSNIGHNY | DNN | GIWDSSLSIVV |
| | | 3289 | 3290 | 3291 | 3292 | | 3294 |
| 98 | VG9B454 | GGSISNYY | IYSSGIT | AGIVGVKGAFAI | SSDVGGYNY | EVS | SSYTSTSVV |
| | | 3323 | 3324 | 3325 | 3326 | | 3328 |
| 99 | VG9B439 | GGSISNYY | IYSSGIT | AGIVGVKGAFAI | SSDVGGYNY | EVS | SSYTSTSVV |
| | | 3357 | 3358 | 3359 | 3360 | | 3362 |
| 100 | VG9B68 | GGTFSTYA | IIPIFGTA | ARGVGWGSDYYYGLDV | QSLVHSDGNTY | KIS | IQATHHPLT |
| | | 3391 | 3392 | 3393 | 3394 | | 3396 |
| 101 | VG9B449 | GGSISNYY | IYSSGIT | AGIVGVKGAFAI | SSDVGGYNY | EVS | SSYTSTSVV |
| | | 3425 | 3426 | 3427 | 3428 | | 3430 |
| 102 | VG9B204 | YGSFSSFY | IYTSGGT | ARWLRAFDY | KLGDKY | QDS | QAWDSSTVV |
| | | 3459 | 3460 | 3461 | 3462 | | 3464 |
| 103 | VG9B459 | GGSISNYY | IYSSGIT | AGIVGVKGAFAI | SSDVGGYNY | EVS | SSYTSTSVV |
| | | 3493 | 3494 | 3495 | 3496 | | 3498 |
| 104 | VG9B157 | GFTFSNHG | ITRGGDTT | TRGPLTVGYAMDY | SLRSYY | GEN | NSRDTGDHHLV |
| | | 3527 | 3528 | 3529 | 3530 | | 3532 |
| 105 | VG9B453 | GGSISNYY | IYSSGIT | AGIVGVKGAFAI | SSDVGGYNY | EVS | SSYTSTSVV |
| | | 3561 | 3562 | 3563 | 3564 | | 3566 |
| 106 | VG9B443 | GGSISNYY | IYSSGIT | AGIVGVKGAFAI | SSDVGGYNY | EVS | SSYTSTSVV |
| | | 3595 | 3596 | 3597 | 3598 | | 3600 |
| 107 | VG9B455 | GGSISNYY | IYSSGIT | AGIVGVKGAFAI | SSDVGGYNY | EVS | SSYTSTSVV |
| | | 3629 | 3630 | 3631 | 3632 | | 3634 |
| 108 | VG9B466 | GGSISNYY | IYSSGIT | AGIVGVKGAFAI | SSDVGGYNY | EVS | SSYTSTSVV |
| | | 3663 | 3664 | 3665 | 3666 | | 3668 |
| 109 | VG9B450 | GGSISNYY | IYSSGIT | AGIVGVKGAFAI | SSDVGGYNY | EVS | SSYTSTSVV |
| | | 3697 | 3698 | 3699 | 3700 | | 3702 |
| 110 | VG9B438 | GGSISNYY | IYSSGIT | AGIVGVKGAFAI | SSDVGGYNY | EVS | SSYTSTSVV |
| | | 3731 | 3732 | 3733 | 3734 | | 3736 |
| 111 | VG9B464 | GGSISNYY | IYSSGIT | AGIVGVKGAFAI | SSDVGGYNY | EVS | SSYTSTSVV |
| | | 3765 | 3766 | 3767 | 3768 | | 3770 |
| 112 | VG9B437 | GGSISNYY | IYSSGIT | AGIVGVKGAFAI | SSDVGGYNY | EVS | SSYTSTSVV |
| | | 3799 | 3800 | 3801 | 3802 | | 3804 |
| 113 | VG9B457 | GGSISNYY | IYSSGIT | AGIVGVKGAFAI | SSDVGGYNY | EVS | SSYTSTSVV |
| | | 3833 | 3834 | 3835 | 3836 | | 3838 |
| 114 | VG9B442 | GGSISNYY | IYSSGIT | AGIVGVKGAFAI | SSDVGGYNY | EVS | SSYTSTSVV |
| | | 3867 | 3868 | 3869 | 3870 | | 3872 |
| 115 | VG9B436 | GGSISNYY | IYSSGIT | AGIVGVKGAFAI | SSDVGGYNY | EVS | SSYTSTSVV |
| | | 3901 | 3902 | 3903 | 3904 | | 3906 |
| 116 | VG9B434 | GGSISNYY | IYSSGIT | AGIVGVKGAFAI | SSDVGGYNY | EVS | SSYTSTSVV |
| | | 3935 | 3936 | 3937 | 3938 | | 3940 |
| 117 | VG9B460 | GGSISNYY | IYSSGIT | AGIVGVKGAFAI | SSDVGGYNY | EVS | SSYTSTSVV |
| | | 3969 | 3970 | 3971 | 3972 | | 3974 |
| 118 | VG9B189 | GFTFSNHG | ITRGGDTT | TRGPLTVGYAMDY | SSDVGGYNY | EVS | NSYAGSNNFEV |
| | | 4003 | 4004 | 4005 | 4006 | | 4008 |
| 119 | VG9B452 | GGSISNYY | IYSSGIT | AGIVGVKGAFAI | SSDVGGYNY | EVS | SSYTSTSVV |
| | | 4037 | 4038 | 4039 | 4040 | | 4042 |
| 120 | VG9B53 | GGSISSYY | IHTIGSI | AMEGVGATNYYYGMAV | QSLLHSNGYNY | LGS | MQALQTPYT |
| | | 4071 | 4072 | 4073 | 4074 | | 4076 |
| 121 | VG9B41 | GYSFTTYW | IDPSDSET | ASNRWLLG | QSLVHSDGNTY | KIS | VQTREFPLT |
| | | 4105 | 4106 | 4107 | 4108 | | 4110 |
| 122 | VG9B136 | GDSVSSNRAA | TYYRSKWYN | ARDLWELREACDI | QGISSY | VAS | QQLNSYPFT |
| | | 4139 | 4140 | 4141 | 4142 | | 4144 |
| 123 | VG9B399 | GFTFSNYD | ISSSSSYI | ARDVGVTTDYYYYGMDV | QSVASSY | GAS | QQYGSSPPYT |
| | | 4173 | 4174 | 4175 | 4176 | | 4178 |
| 124 | VG9B369 | GFSLSTIGVG | IYWNDDK | AHSHDWVHAFDI | QDISHY | DAS | QQYDNFPLT |
| | | 4207 | 4208 | 4209 | 4210 | | 4212 |
| 125 | VG9B144 | GYTFTGYY | INPNSGAT | AREDDAFDV | NLRNYY | GKN | NSRDSSGNHVV |
| | | 4241 | 4242 | 4243 | 4244 | | 4246 |
| 126 | VG9B410 | GFTFSNYG | ISSGSSYK | ARDPVVTEYYYYGMDV | QGINSW | AAS | QQANSFPWT |
| | | 4275 | 4276 | 4277 | 4278 | | 4280 |
| 127 | VG9B386 | GFTFSSYS | IGSSSTYI | ARDGETGGFDY | QSISRSY | GPS | QQYGSLPLT |
| | | 4309 | 4310 | 4311 | 4312 | | 4314 |
| 128 | VG9B125 | GFTFSSYD | ISSSSTAK | AREDIVVVTPILQH | QSVSKD | DAS | QQRINWPLFT |
| | | 4343 | 4344 | 4345 | 4346 | | 4348 |
| 129 | VG9B112 | GDSVSSNSAA | TYYRSKWFN | ARGEWGLRDAFDI | QGIHSY | VAS | QQLNSYPWT |
| | | 4377 | 4378 | 4379 | 4380 | | 4382 |
| 130 | VG9B371 | RFTLSSYD | ISSSSSYI | ARDRGVGGTDYYYYGLDV | QSVSSSY | GAS | QQYGSSPPYT |
| | | 4411 | 4412 | 4413 | 4414 | | 4416 |
| 131 | VG9B387 | GFTFTGYY | INPNSGYT | ARLDDAFDV | SLRNYY | GKN | NSRDSSGNHWV |
| | | 4445 | 4446 | 4447 | 4448 | | 4450 |
| 132 | VG9B47 | GGSFSNYA | IIPFFGTP | STGGGYGDYDYYYGINV | QSLVHSDGNTY | KIS | MQAKEFPLT |
| | | 4479 | 4480 | 4481 | 4482 | | 4484 |
| 133 | VG9B379 | GYTFTGDY | INPNSGGT | AREGGVAPAAPDAFDI | SLRSYY | GKN | NSRDSSGNHRV |
| | | 4513 | 4514 | 4515 | 4516 | | 4518 |
| 134 | VG9B447 | GGSISNYY | IYSSGIT | AGIVGVKGAFAI | SSDVGGYNY | EVS | SSYTSTSVV |
| | | 4547 | 4548 | 4549 | 4550 | | 4552 |
| 135 | VG9B392 | GYTFTGYY | INPNSGYT | AREDDAFDI | SLRSYY | GKN | NSRDNSGNHVV |
| | | 4581 | 4582 | 4583 | 4584 | | 4586 |
| 136 | VG9B96 | GGTFSNYA | IIPIFSAG | SSNSGTYYDYYYGMDV | QSLVHSDGNTY | KIF | MQAKQFPLT |
| | | 4615 | 4616 | 4617 | 4618 | | 4620 |
| 137 | VG9B77 | GGTFSSYA | IIPFFGTA | ATATVTTDYYYGMDV | QSLVHSDGNTY | EIS | MQARQFPLT |
| | | 4649 | 4650 | 4651 | 4652 | | 4654 |

Variable Region Cloning. Both RNA purified by QIAGEN kit (RNEASY Plus Mini Kit) and B cells lysate were used for cDNA synthesis using the Smarter cDNA synthesis kit (Clontech, Mount View, CA). To facilitate cDNA synthesis, oligodT was used to prime reverse transcription of all messenger RNAs followed by “5′ capping” with a Smarter IIA oligonucleotide. Subsequent amplification of the VH and VL fragments was performed using a two-step PCR amplification using 5′ primers targeting the SMARTER IIA cap and 3′ primers targeting consensus regions in CH1. Briefly, each 50 μl PCR reaction consists of 20 pM of forward and reverse primer mixes, 25 μl of PRIMIESTAR MAX DNA polymerase premix (Clontech), 2 μl of unpurified cDNA, and 21 μl of double-distilled H2O. The cycling program started at 94° C. for 3 min., followed by 35 cycles (94° C. for 30 sec., 55° C. for 1 min., 68° C. for 1 min.), and ended at 72° C. for 7 min. The second round PCR was performed with VL and VH second round primers containing 15 bp complementary extensions that “overlap” respective regions in their respective Lonza mother vector (VH and VL). Second round PCR was performed with the following program: 94° C. for 3 min.; 35 cycles (94° C. for 30 sec., 55° C. for 1 min., 68° C. for 1 min.), and ended at 72° C. for 7 min. IN-FUSION® HD Cloning Kit (Clonetech, U.S.A.) was used for directional cloning of VL gene into Lonza huIgK or Lambda vector and VH gene into Lonza huIgG1 vector. To facilitate IN-FUSION® HD Cloning, PCR products were treated with Cloning Enhancer before IN-FUSION HD Cloning. Cloning and transformation were performed according to manufacturer's protocol (Clonetech, U.S.A.). Mini-prep DNAs were subjected to Sanger sequencing to confirm that complete V-gene fragments were obtained.

### 1.3 Binding Activity of TRGV9, TRDV2 and TRGDC Antibodies on Target Cell Lines.

Binding of antibodies to human γδ T cells or SKW cell lines expressing Vγ9/Vδ2, Vγ9/Vδ2 or Vγ5/Vδ2 was carried out by flow cytometry. Briefly, the cells were centrifuged at 300×g for 5 minutes and the pellet was resuspended in 34 ml fresh media (in RPMI+10% FBS). 50 μL of the cells were then plated (44K cells/well) into assay plates. Samples were incubated for one hour at 37° C. in tissue culture (TC) incubator followed by wash steps by adding 100 μl FACS staining buffer. Secondary detention antibodies (A647 conjugated anti-human IgG Fc specific antibody (2 pg/mL) along with AF488 anti mouse CD3, clone OKT3 at 1 μg/mL) were added at 50 μL/well to appropriate wells according to the plate map. After incubating for 60 minutes at 37° C. in the TC incubator, cells were again washed as described above. This is followed by resuspension in 20 μL/well running buffer containing 1:1000 dilution of Sytox blue dead cell stain and the plates were run on iQue Screener.

Gating and data analysis: Human ant-human Vγ9 antibody (VG9B2 mAb at 10 nM) was used to mark the gating of positive population. All cells were first gated for singlets on SCS-A vs. SCS-H dot plot, live/dead followed by CD3 positive population, and this subset of CD3 positive population was gated for Vγ9 positive binding using positive control mAb. Cells were also assessed for cell binding of control mAbs by RL1 (A647) Geomeans from the live cell population. Advanced metrics were used and normalization metrics were chosen to calculate fold over background wells containing secondary antibody alone (designated as ‘background’ within ForeCyt settings). The results were exported to GeneData screener to graph and summarize results, which are shown in Table 8.

TABLE 8
Antibody Binding

| | | | | SKW-Cell Lines | | |
|---|---|---|---|---|---|---|
| | | | | DRC Binding | DRC Binding | DRC Binding |
| | | | | Flow 37° C.: | Flow 37° C.: | Flow 37° C.: |
| | | | Human | AG000003284 | AG000003287 | AG000003285 |
| | | | γδ T cells | (SKW3_4 | (SKW3 | (SKW3 |
| | | | DRC Binding | expressing | expressing | expressing |
| | | | Flow 37° C.: | Vg9/Vd2) | Vg9/Vd1) | Vg5/Vd2) |
| | | | AG000003021 | [CLI000001324 | [CLI000001326 | [CLI000001325 |
| | Protein | | (γδ T cells) | (C3819A)] | (C3821A)] | (C3820A)] |
| # | Name | Specificity | Max. Activity | Max. Activity | Max. Activity | Max. Activity |
| 1 | VG9B54 | Vg9 | 4287.92 | 165.54 | 348.47 | 2.49 |
| 2 | VG9B121 | Vg9 | 3666.93 | 160.56 | 360.43 | 18.44 |
| 3 | VG9B429 | Vg9 | 3637.59 | 177.29 | 420.69 | 1.76 |
| 4 | VG9B370 | Vg9 | 3529.08 | 183.63 | 424.06 | 2.63 |
| 5 | VG9B80 | Vg9 | 3480.37 | 133.20 | 313.72 | 2.24 |
| 6 | VG9B414 | Vg9 | 3295.02 | 195.62 | 498.04 | 10.02 |
| 7 | VG9B195 | Vg9 | 3257.31 | 119.35 | 299.67 | 10.31 |
| 8 | VG9B140 | Vg9 | 3226.11 | 133.46 | 325.32 | 2.78 |
| 9 | VG9B426 | Vg9 | 3212.79 | 154.44 | 339.73 | 2.40 |
| 10 | VG9B46 | Vg9 | 3207.94 | 164.92 | 349.12 | 1.95 |
| 11 | VG9B416 | Vg9 | 3122.34 | 180.59 | 420.36 | 1.91 |
| 12 | VG9B69 | Vg9 | 3109.88 | 147.17 | 341.06 | 3.35 |
| 13 | VG9B415 | Vg9 | 3077.36 | 173.88 | 411.15 | 2.66 |
| 14 | VG9B104 | Vg9 | 3052.98 | 147.85 | 358.46 | 1.90 |
| 15 | VG9B198 | Vg9 | 3042.49 | 160.00 | 372.45 | 1.89 |
| 16 | VG9B463 | Vg9 | 3032.24 | 144.29 | 321.13 | 4.19 |
| 17 | VG9B469 | Vg9 | 3003.65 | 168.31 | 399.44 | 1.44 |
| 18 | VG9B428 | Vg9 | 2984.79 | 162.05 | 346.00 | 2.86 |
| 19 | VG9B430 | Vg9 | 2965.48 | 142.74 | 324.73 | 1.49 |
| 20 | VG9B423 | Vg9 | 2963.87 | 155.60 | 344.61 | 2.87 |
| 21 | VG9B98 | Vg9 | 2879.14 | 153.54 | 343.86 | 2.47 |
| 22 | VG9B73 | Vg9 | 2844.52 | 117.98 | 317.45 | 1.79 |
| 23 | VG9B133 | Vg9 | 2835.38 | 143.97 | 307.41 | 5.97 |
| 24 | VG9B368 | Vg9 | 2784.27 | 185.70 | 398.79 | 3.23 |
| 25 | VG9B424 | Vg9 | 2782.65 | 154.63 | 341.58 | 19.79 |
| 26 | VG9B427 | Vg9 | 2728.10 | 151.51 | 344.45 | 1.82 |
| 27 | VG9B417 | Vg9 | 2698.70 | 169.62 | 424.38 | 2.15 |
| 28 | VG9B58 | Vg9 | 2694.07 | 159.70 | 350.03 | 1.49 |
| 29 | VG9B419 | Vg9 | 2689.29 | 171.96 | 400.28 | 1.87 |
| 30 | VG9B425 | Vg9 | 2664.04 | 155.86 | 368.27 | 1.87 |
| 31 | VG9B143 | Vg9 | 2629.19 | 97.35 | 241.41 | 1.07 |
| 32 | VG9B418 | Vg9 | 2625.01 | 190.37 | 397.53 | 3.80 |
| 33 | VG9B472 | Vg9 | 2618.27 | 171.46 | 327.75 | 2.22 |
| 34 | VG9B421 | Vg9 | 2613.21 | 164.91 | 357.78 | 2.16 |
| 35 | VG9B88 | Vg9 | 2559.14 | 153.54 | 345.70 | 18.78 |
| 36 | VG9B384 | Vg9 | 2546.76 | 120.75 | 289.57 | 18.67 |
| 37 | VG9B413 | Vg9 | 2472.53 | 184.38 | 405.73 | 4.47 |
| 38 | VG9B36 | Vg9 | 2411.74 | 156.79 | 354.36 | 2.45 |
| 39 | VG9B403 | Vg9 | 2388.25 | 192.50 | 426.04 | 2.43 |
| 40 | VG9B191 | Vg9 | 2366.62 | 155.61 | 381.04 | 17.18 |
| 41 | VG9B44 | Vg9 | 2317.34 | 169.68 | 375.29 | 2.13 |
| 42 | VG9B67 | Vg9 | 2267.90 | 128.38 | 317.80 | 1.14 |
| 43 | VG9B402 | Vg9 | 2245.94 | 175.93 | 390.71 | 2.50 |
| 44 | VG9B127 | Vg9 | 2225.81 | 91.86 | 317.54 | 1.78 |
| 45 | VG9B137 | Vg9 | 2199.84 | 133.26 | 316.80 | 1.84 |
| 46 | VG9B33 | Vg9 | 2093.60 | 173.44 | 377.35 | 2.85 |
| 47 | VG9B162 | Vg9 | 1855.16 | 96.27 | 249.35 | 2.44 |
| 48 | VG9B152 | Vg9 | 1851.56 | 140.95 | 351.66 | 1.64 |
| 49 | VG9B64 | Vg9 | 1688.57 | 100.31 | 259.64 | 2.22 |
| 50 | VG9B21 | Vg9 | 1672.33 | 39.71 | 121.09 | 1.86 |
| 51 | VG9B128 | Vg9 | 1483.38 | 120.69 | 215.26 | 11.39 |
| 52 | VG9B66 | Vg9 | 1223.28 | 33.87 | 179.02 | 1.04 |
| 53 | VG9B32 | Vg9 | 1211.08 | 40.02 | 108.12 | 2.52 |
| 54 | VG9B57 | Vg9 | 1206.73 | 30.76 | 75.94 | 1.04 |
| 55 | VG9B135 | Vg9 | 577.24 | 15.10 | 59.87 | 2.65 |
| 56 | VG9B60 | Vg9 | 567.17 | 16.70 | 40.58 | 1.42 |
| 57 | VG9B409 | Vg9 | 410.62 | 176.35 | 408.24 | 8.45 |
| 58 | VG9B411 | Vg9 | 396.41 | 19.12 | 24.89 | 2.76 |
| 59 | VG9B129 | Vg9 | 271.32 | 10.36 | 23.35 | 1.13 |
| 60 | VG9B396 | Vd2 | 4543.78 | 154.68 | 2.19 | 222.19 |
| 61 | VG9B470 | Vd2 | 4273.42 | 166.74 | 1.59 | 231.07 |
| 62 | VG9B111 | Vd2 | 3433.67 | 116.94 | 1.92 | 154.43 |
| 63 | VG9B169 | Vd2 | 3246.37 | 165.28 | 2.21 | 175.26 |
| 64 | VG9B639 | Vd2 | 3236.63 | 155.28 | 1.29 | 185.68 |
| 65 | VG9B201 | Vd2 | 3170.12 | 152.35 | 2.74 | 204.61 |
| 66 | VG9B161 | Vd2 | 2819.85 | 180.14 | 1.43 | 210.72 |
| 67 | VG9B383 | Vd2 | 2778.28 | 183.98 | 1.36 | 283.96 |
| 68 | VG9B382 | Vd2 | 2775.57 | 176.98 | 1.04 | 275.40 |
| 69 | VG9B156 | Vd2 | 2698.68 | 152.58 | 1.60 | 169.64 |
| 70 | VG9B205 | Vd2 | 2689.76 | 153.49 | 2.04 | 161.98 |
| 71 | VG9B86 | Vd2 | 2670.16 | 137.94 | 1.16 | 206.91 |
| 72 | VG9B154 | Vd2 | 2592.95 | 152.88 | 1.09 | 92.70 |
| 73 | VG9B159 | Vd2 | 2505.10 | 123.26 | 1.58 | 11.37 |
| 74 | VG9B465 | Vd2 | 2500.83 | 148.03 | 7.28 | 214.03 |
| 75 | VG9B194 | Vd2 | 2470.37 | 151.47 | 6.29 | 148.31 |
| 76 | VG9B182 | Vd2 | 2365.20 | 159.31 | 1.68 | 195.27 |
| 77 | VG9B173 | Vd2 | 2362.86 | 181.80 | 1.82 | 25.36 |
| 78 | VG9B87 | Vd2 | 2331.26 | 138.98 | 1.40 | 51.63 |
| 79 | VG9B208 | Vd2 | 2306.11 | 176.93 | 2.15 | 203.33 |
| 80 | VG9B372 | Vd2 | 2293.95 | 127.30 | 1.02 | 171.26 |
| 81 | VG9B186 | Vd2 | 2287.42 | 142.50 | 1.20 | 66.88 |
| 82 | VG9B177 | Vd2 | 2247.18 | 161.00 | 1.28 | 192.81 |
| 83 | VG9B114 | Vd2 | 1975.60 | 176.44 | 8.38 | 226.28 |
| 84 | VG9B147 | Vd2 | 1890.32 | 30.48 | 1.59 | 69.75 |
| 85 | VG9B65 | Vd2 | 1819.09 | 88.75 | 8.50 | 122.51 |
| 86 | VG9B81 | Vd2 | 1771.18 | 42.23 | 1.17 | 77.02 |
| 87 | VG9B203 | Vd2 | 1753.37 | 143.32 | 1.30 | 193.78 |
| 88 | VG9B380 | Vd2 | 1749.32 | 163.87 | 11.60 | 232.97 |
| 89 | VG9B103 | Vd2 | 1675.38 | 96.41 | 1.79 | 236.84 |
| 90 | VG9B462 | Vd2 | 1541.64 | 140.24 | 21.55 | 141.65 |
| 91 | VG9B461 | Vd2 | 1539.12 | 148.91 | 9.04 | 153.18 |
| 92 | VG9B106 | Vd2 | 1524.56 | 10.48 | 1.20 | 64.28 |
| 93 | VG9B115 | Vd2 | 1522.89 | 41.89 | 1.49 | 66.95 |
| 94 | VG9B27 | Vd2 | 1446.95 | 137.76 | 5.83 | 208.79 |
| 95 | VG9B458 | Vd2 | 1412.02 | 147.86 | 4.38 | 123.29 |
| 96 | VG9B131 | Vd2 | 1403.08 | 58.11 | 2.38 | 91.72 |
| 97 | VG9B163 | Vd2 | 1346.41 | 57.19 | 10.63 | 48.91 |
| 98 | VG9B454 | Vd2 | 1244.69 | 180.68 | 29.37 | 158.37 |
| 99 | VG9B439 | Vd2 | 1244.61 | 169.53 | 1.84 | 162.87 |
| 100 | VG9B68 | Vd2 | 1192.87 | 124.09 | 2.08 | 207.12 |
| 101 | VG9B449 | Vd2 | 1178.94 | 164.57 | 1.47 | 158.60 |
| 102 | VG9B204 | Vd2 | 1129.50 | 99.65 | 1.50 | 169.49 |
| 103 | VG9B459 | Vd2 | 1087.45 | 156.84 | 7.55 | 137.62 |
| 104 | VG9B157 | Vd2 | 1070.92 | 10.96 | 0.96 | 11.69 |
| 105 | VG9B453 | Vd2 | 1037.24 | 171.23 | 1.69 | 162.89 |
| 106 | VG9B443 | Vd2 | 1005.02 | 171.26 | 5.90 | 150.44 |
| 107 | VG9B455 | Vd2 | 1000.40 | 169.15 | 1.33 | 153.89 |
| 108 | VG9B466 | Vd2 | 945.82 | 139.31 | 1.76 | 126.03 |
| 109 | VG9B450 | Vd2 | 927.78 | 167.57 | 1.13 | 159.63 |
| 110 | VG9B438 | Vd2 | 895.07 | 164.78 | 1.74 | 165.98 |
| 111 | VG9B464 | Vd2 | 886.16 | 145.74 | 1.73 | 115.48 |
| 112 | VG9B437 | Vd2 | 885.19 | 164.62 | 1.30 | 153.25 |
| 113 | VG9B457 | Vd2 | 864.60 | 179.57 | 0.93 | 157.79 |
| 114 | VG9B442 | Vd2 | 856.24 | 159.04 | 1.48 | 158.00 |
| 115 | VG9B436 | Vd2 | 812.47 | 162.02 | 1.88 | 169.69 |
| 116 | VG9B434 | Vd2 | 803.78 | 171.06 | 1.48 | 159.57 |
| 117 | VG9B460 | Vd2 | 767.37 | 138.23 | 1.47 | 129.82 |
| 118 | VG9B189 | Vd2 | 738.68 | 46.15 | 1.46 | 21.54 |
| 119 | VG9B452 | Vd2 | 686.75 | 181.12 | 2.10 | 166.55 |
| 120 | VG9B53 | Vd2 | 536.30 | 7.34 | 0.93 | 109.60 |
| 121 | VG9B41 | Vd2 | 137.19 | 11.43 | 2.18 | 16.08 |
| 122 | VG9B136 | g/d constant | 2684.58 | 180.54 | 288.27 | 226.18 |
| 123 | VG9B399 | g/d constant | 2478.03 | 214.03 | 435.82 | 206.50 |
| 124 | VG9B369 | g/d constant | 2429.10 | 258.91 | 319.93 | 241.30 |
| 125 | VG9B144 | g/d constant | 2425.86 | 123.44 | 204.70 | 193.74 |
| 126 | VG9B410 | g/d constant | 2253.41 | 222.69 | 434.89 | 54.42 |
| 127 | VG9B386 | g/d constant | 2113.79 | 210.54 | 388.92 | 92.74 |
| 128 | VG9B125 | g/d constant | 1992.68 | 126.84 | 277.32 | 194.19 |
| 129 | VG9B112 | g/d constant | 1991.02 | 227.15 | 328.28 | 217.02 |
| 130 | VG9B371 | g/d constant | 1970.39 | 225.19 | 430.68 | 130.30 |
| 131 | VG9B387 | g/d constant | 1965.44 | 200.61 | 434.64 | 261.40 |
| 132 | VG9B47 | g/d constant | 1558.28 | 162.35 | 308.26 | 211.59 |
| 133 | VG9B379 | g/d constant | 1458.49 | 160.15 | 294.95 | 260.37 |
| 134 | VG9B447 | g/d constant | 1417.79 | 196.00 | 94.00 | 161.41 |
| 135 | VG9B392 | g/d constant | 1247.94 | 154.25 | 295.22 | 281.15 |
| 136 | VG9B96 | g/d constant | 1022.01 | 126.04 | 244.90 | 223.89 |
| 137 | VG9B77 | g/d constant | 771.90 | 102.46 | 137.09 | 147.80 |

Cytokine and effector molecule analysis. Cytokines and effector molecules were assessed both intracellularly and in the cell culture supernatants. For intracellular cytokine and effector molecules assessment by flow cytometry, cells were initially surface stained with indicated monoclonal antibodies and washed twice with wash buffer. Stained cells were fixed and permeabilized using BD Fix/Perm kit (BD biosciences) per manufacturer's instructions. Permeabilized cells were probed with monoclonal antibodies against intracellular cytokines (TNFα, IFNγ) or effector molecules (Granzyme B) for 30 minutes at 4° C. Cells were washed twice and acquired on Novocyte flow cytometer. FMO (Fluorescence Minus One) controls were used to establish the gating for cytokines. For assessing cytokines in cell culture supernatant, cell culture supernatants were collected on indicated time points and subjected to quantification using customized human magnetic Luminex assay 15-plex kit (R&D Systems, Minneapolis, USA), as per the manufacturer's instructions. Quantification of the cytokines was carried out in MAGPIX multiplex detection system with xPONENT software. Results are shown in Table 9.

Activated T Cell Surface Marker Profiles. Flow cytometery was utilized to asses the percentage of Vγ9+ cells expressing certain T cell activation markers, including CD25 and CD71 expression, as well as the percentage of Vγ9 T cells in the cell population. Briefly, 48-well plates were coated with 200 μl of antibodies (1 pg/ml) and control antibody (VG7A5 1 μg/ml) in PBS overnight at 40° C. PBMCs were thawed and resuspended in RPMI+10% FBS media. They were then labeled with CTV dye to track proliferation. CTV labeled PBMCs were plated 2×10⁶ in 250 μl media per well of the antibody coated plates and incubated at 37° C. for 2 days. Supernatant was collected on day 2 for Luminex analysis (IFNγ, TNFα, Granzyme B). The cells were spun down and transferred to IL-2 containing media (100 IU/ml) and allowed to rest for 3 days followed by staining for CD25, CD71, IFNγ and Granzyme B after 3 days. The results are shown in Table 9.

TABLE 9
Cell Cytokine and Surface Marker Profiles

| | | | | | | Flow Staining | | |
|---|---|---|---|---|---|---|---|---|
| | Protein | Luninex (supernatant) | | | | CD25+ | CD71+ | Vg9 T |
| # | Name | TNFα | IL6 | IFNγ | Granzyme B | (Vg9+ cells) | (Vg9+ cells) | cell % |
| 1 | VG9B54 | 6096 | 761 | 1601 | 3103 | 32.4 | 93.3 | 10.5 |
| 2 | VG9B121 | 96 | 161 | 429 | 1352 | 50.9 | 98.7 | 16.1 |
| 3 | VG9B429 | 583 | 816 | 1249 | 2148 | 25.8 | 91.6 | 3.0 |
| 4 | VG9B370 | 2297 | 541 | 2839 | 5023 | 44.9 | 98.6 | 8.0 |
| 5 | VG9B80 | 4914 | 385 | 1364 | 3009 | 49.6 | 98.3 | 5.2 |
| 6 | VG9B414 | 186 | 219 | 476 | 1489 | 24.8 | 88.1 | 4.0 |
| 7 | VG9B195 | 61 | 166 | 303 | 1064 | 15.8 | 86.9 | 3.7 |
| 8 | VG9B140 | 3517 | 602 | 796 | 2161 | 20.4 | 70.6 | 3.9 |
| 9 | VG9B426 | 789 | 621 | 2348 | 4586 | 36.5 | 98.8 | 7.6 |
| 10 | VG9B46 | 5256 | 375 | 1154 | 3174 | 37.9 | 95.8 | 4.4 |
| 11 | VG9B416 | 87 | 225 | 1175 | 3390 | 46.4 | 98.8 | 13.9 |
| 12 | VG9B69 | 1099 | 591 | 2070 | 3238 | 40.4 | 97.7 | 8.8 |
| 13 | VG9B415 | 193 | 403 | 1213 | 5157 | 31.5 | 96.9 | 5.3 |
| 14 | VG9B104 | 4132 | 1092 | 2441 | 5290 | 41.4 | 95.2 | 5.1 |
| 15 | VG9B198 | 66 | 199 | 324 | 1103 | 23.0 | 91.2 | 3.6 |
| 16 | VG9B463 | 2109 | 100 | 364 | 965 | 26.0 | 87.4 | 4.1 |
| 17 | VG9B469 | 173 | 440 | 1585 | 3696 | 29.9 | 98.5 | 7.8 |
| 18 | VG9B428 | 390 | 613 | 1995 | 4125 | 37.0 | 98.1 | 5.0 |
| 19 | VG9B430 | 125 | 372 | 1491 | 2515 | 37.5 | 96.2 | 10.7 |
| 20 | VG9B423 | 229 | 590 | 1432 | 3376 | 40.3 | 98.9 | 13.9 |
| 21 | VG9B98 | 2007 | 294 | 305 | 1424 | 29.4 | 90.5 | 7.2 |
| 22 | VG9B73 | 136 | 359 | 1051 | 3372 | 30.6 | 94.1 | 13.8 |
| 23 | VG9B133 | 123 | 478 | 1167 | 3475 | 21.8 | 91.4 | 5.8 |
| 24 | VG9B368 | 118 | 260 | 1484 | 3314 | 42.3 | 98.2 | 11.5 |
| 25 | VG9B424 | 174 | 931 | 1405 | 3964 | 36.7 | 98.8 | 12.1 |
| 26 | VG9B427 | 220 | 340 | 2487 | 4676 | 50.6 | 99.1 | 13.6 |
| 27 | VG9B417 | 202 | 1409 | 2873 | 5568 | 49.6 | 98.8 | 10.9 |
| 28 | VG9B58 | 3454 | 572 | 1599 | 3839 | 33.9 | 91.8 | 6.8 |
| 29 | VG9B419 | 216 | 362 | 2429 | 4250 | 34.2 | 98.1 | 5.0 |
| 30 | VG9B425 | 127 | 612 | 1356 | 3740 | 39.3 | 98.6 | 15.6 |
| 31 | VG9B143 | 32 | 113 | 155 | 1086 | 11.4 | 87.5 | 6.2 |
| 32 | VG9B418 | 163 | 279 | 2182 | 3886 | 46.9 | 99.2 | 13.3 |
| 33 | VG9B472 | 84 | 394 | 575 | 2780 | 23.4 | 96.6 | 6.3 |
| 34 | VG9B421 | 255 | 604 | 2454 | 6129 | 36.7 | 98.7 | 7.1 |
| 35 | VG9B88 | 1263 | 551 | 1531 | 2377 | 15.1 | 88.1 | 4.1 |
| 36 | VG9B384 | 3744 | 610 | 1099 | 2301 | 26.4 | 89.9 | 3.4 |
| 37 | VG9B413 | 229 | 1961 | 2830 | 5795 | 44.9 | 98.8 | 13.3 |
| 38 | VG9B36 | 188 | 607 | 2581 | 3893 | 29.1 | 89.4 | 7.8 |
| 39 | VG9B403 | 102 | 175 | 1388 | 3017 | 36.2 | 97.5 | 7.2 |
| 40 | VG9B191 | 7323 | 400 | 1385 | 3373 | 42.4 | 93.6 | 5.2 |
| 41 | VG9B44 | 198 | 587 | 1147 | 3031 | 45.4 | 98.8 | 14.4 |
| 42 | VG9B67 | 144 | 365 | 972 | 2553 | 28.5 | 87.4 | 14.0 |
| 43 | VG9B402 | 376 | 433 | 2367 | 4497 | 45.5 | 99.1 | 9.5 |
| 44 | VG9B127 | 709 | 348 | 1173 | 2453 | 37.3 | 96.5 | 11.2 |
| 45 | VG9B137 | 30 | 8 | 44 | 36 | 8.7 | 77.3 | 3.6 |
| 46 | VG9B33 | 85 | 240 | 749 | 1994 | 28.0 | 95.2 | 14.0 |
| 47 | VG9B162 | 329 | 54 | 57 | 481 | 9.7 | 70.3 | 2.7 |
| 48 | VG9B152 | 51 | 118 | 344 | 1060 | 8.1 | 81.9 | 3.0 |
| 49 | VG9B64 | 46 | 122 | 383 | 863 | 21.4 | 79.6 | 4.0 |
| 50 | VG9B21 | 3329 | 459 | 1660 | 3153 | 27.5 | 91.8 | 7.1 |
| 51 | VG9B128 | 3964 | 304 | 1242 | 4854 | 36.6 | 97.4 | 9.6 |
| 52 | VG9B66 | 405 | 145 | 162 | 470 | 25.4 | 84.6 | 3.8 |
| 53 | VG9B32 | 10 | 11 | 44 | 68 | 19.1 | 87.3 | 5.1 |
| 54 | VG9B57 | 349 | 147 | 29 | 468 | 17.1 | 77.4 | 4.0 |
| 55 | VG9B135 | 225 | 717 | 57 | 270 | 20.6 | 77.7 | 4.3 |
| 56 | VG9B60 | 2425 | 269 | 280 | 912 | 9.1 | 73.0 | 2.9 |
| 57 | VG9B409 | 190 | 194 | 195 | 585 | 18.7 | 80.6 | 3.2 |
| 58 | VG9B411 | 25 | 88 | 51 | 115 | 10.8 | 75.4 | 3.0 |
| 59 | VG9B129 | 192 | 94 | 36 | 305 | 16.4 | 73.4 | 3.6 |
| 60 | VG9B396 | 908 | 172 | 531 | 1388 | 44.7 | 92.7 | 2.7 |
| 61 | VG9B470 | 3659 | 554 | 828 | 2231 | 43.5 | 92.6 | 2.3 |
| 62 | VG9B111 | 42 | 32 | 44 | 98 | 27.8 | 84.4 | 8.8 |
| 63 | VG9B169 | 722 | 399 | 577 | 2381 | 30.3 | 95.3 | 2.0 |
| 64 | VG9B639 | 6081 | 755 | 1397 | 4289 | 37.8 | 94.7 | 4.3 |
| 65 | VG9B201 | 460 | 77 | 225 | 987 | 21.8 | 89.4 | 4.2 |
| 66 | VG9B161 | 1285 | 286 | 620 | 1671 | 31.3 | 94.5 | 5.4 |
| 67 | VG9B383 | 687 | 379 | 1242 | 6027 | 29.7 | 91.3 | 6.4 |
| 68 | VG9B382 | 49 | 46 | 440 | 1640 | 32.7 | 93.9 | 6.0 |
| 69 | VG9B156 | 14 | 37 | 44 | 493 | 27.5 | 93.7 | 6.4 |
| 70 | VG9B205 | 224 | 66 | 99 | 800 | 22.0 | 91.4 | 2.4 |
| 71 | VG9B86 | 2254 | 186 | 503 | 1052 | 27.4 | 94.1 | 4.7 |
| 72 | VG9B154 | 9 | 4 | 44 | 181 | 26.3 | 93.7 | 6.9 |
| 73 | VG9B159 | 17 | 42 | 44 | 711 | 10.3 | 85.7 | 3.5 |
| 74 | VG9B465 | 132 | 126 | 795 | 1472 | 28.7 | 94.0 | 3.6 |
| 75 | VG9B194 | 276 | 40 | 44 | 316 | 17.0 | 82.6 | 3.7 |
| 76 | VG9B182 | 1040 | 209 | 938 | 2180 | 37.9 | 91.0 | 2.5 |
| 77 | VG9B173 | 17 | 104 | 83 | 766 | 22.9 | 90.4 | 5.1 |
| 78 | VG9B87 | 674 | 117 | 154 | 652 | 32.9 | 94.5 | 4.5 |
| 79 | VG9B208 | 22 | 31 | 68 | 618 | 16.8 | 91.7 | 3.8 |
| 80 | VG9B372 | 73 | 105 | 149 | 828 | 11.2 | 89.3 | 4.0 |
| 81 | VG9B186 | 429 | 56 | 68 | 327 | 13.3 | 78.3 | 3.4 |
| 82 | VG9B177 | 99 | 117 | 60 | 768 | 22.8 | 88.6 | 2.4 |
| 83 | VG9B114 | 6999 | 731 | 1081 | 3901 | 53.7 | 98.3 | 9.7 |
| 84 | VG9B147 | 58 | 81 | 44 | 307 | 12.0 | 89.0 | 3.0 |
| 85 | VG9B65 | 42 | 190 | 179 | 1040 | 24.6 | 96.4 | 7.2 |
| 86 | VG9B81 | 1147 | 144 | 138 | 500 | 27.4 | 79.7 | 8.4 |
| 87 | VG9B203 | 22 | 30 | 44 | 525 | 19.0 | 90.2 | 4.5 |
| 88 | VG9B380 | 15 | 21 | 44 | 228 | 15.5 | 84.3 | 4.2 |
| 89 | VG9B103 | 4760 | 298 | 889 | 3212 | 46.6 | 96.4 | 5.0 |
| 90 | VG9B462 | 7463 | 819 | 1461 | 2639 | 28.5 | 83.6 | 2.7 |
| 91 | VG9B461 | 7904 | 748 | 1353 | 3166 | 34.4 | 85.3 | 2.7 |
| 92 | VG9B106 | 119 | 39 | 44 | 287 | 12.2 | 73.9 | 3.5 |
| 93 | VG9B115 | 204 | 104 | 44 | 186 | 26.8 | 83.5 | 3.5 |
| 94 | VG9B27 | 3537 | 274 | 1492 | 4123 | 35.4 | 92.6 | 4.0 |
| 95 | VG9B458 | 4324 | 384 | 1026 | 2230 | 27.6 | 84.5 | 3.1 |
| 96 | VG9B131 | 371 | 76 | 91 | 429 | 18.8 | 80.3 | 3.4 |
| 97 | VG9B163 | 9 | 5 | 44 | 41 | 9.7 | 75.9 | 3.7 |
| 98 | VG9B454 | 2063 | 458 | 1215 | 2475 | 39.9 | 92.7 | 5.0 |
| 99 | VG9B439 | 190 | 140 | 167 | 760 | 13.8 | 86.0 | 3.9 |
| 100 | VG9B68 | 6831 | 539 | 1544 | 4204 | 35.6 | 93.5 | 9.3 |
| 101 | VG9B449 | 266 | 131 | 256 | 1089 | 16.3 | 87.2 | 3.5 |
| 102 | VG9B204 | 3255 | 230 | 698 | 1878 | 31.8 | 91.4 | 4.3 |
| 103 | VG9B459 | 1589 | 409 | 1060 | 2322 | 24.2 | 86.6 | 2.9 |
| 104 | VG9B157 | 74 | 606 | 36 | 115 | 13.1 | 78.3 | 3.3 |
| 105 | VG9B453 | 5756 | 612 | 1670 | 3297 | 29.7 | 87.9 | 3.0 |
| 106 | VG9B443 | 5106 | 860 | 1174 | 2548 | 33.6 | 86.0 | 3.8 |
| 107 | VG9B455 | 7052 | 1576 | 2726 | 9278 | 24.2 | 77.3 | 2.6 |
| 108 | VG9B466 | 5870 | 558 | 1194 | 2375 | 21.7 | 84.6 | 2.6 |
| 109 | VG9B450 | 1510 | 293 | 560 | 1379 | 26.1 | 83.9 | 3.5 |
| 110 | VG9B438 | 1419 | 387 | 653 | 2365 | 28.2 | 86.4 | 4.2 |
| 111 | VG9B464 | 5479 | 392 | 1584 | 2486 | 26.6 | 86.2 | 3.3 |
| 112 | VG9B437 | 311 | 899 | 666 | 1489 | 20.4 | 85.4 | 3.7 |
| 113 | VG9B457 | 1201 | 1066 | 778 | 1836 | 24.0 | 83.2 | 3.4 |
| 114 | VG9B442 | 617 | 542 | 322 | 1306 | 18.4 | 82.0 | 3.9 |
| 115 | VG9B436 | 271 | 149 | 230 | 1080 | 21.5 | 87.0 | 3.9 |
| 116 | VG9B434 | 478 | 985 | 900 | 4803 | 20.5 | 85.3 | 4.2 |
| 117 | VG9B460 | 333 | 528 | 410 | 1252 | 18.5 | 88.0 | 3.2 |
| 118 | VG9B189 | 64 | 30 | 44 | 230 | 11.6 | 80.0 | 3.5 |
| 119 | VG9B452 | 1891 | 273 | 1037 | 1828 | 26.4 | 84.9 | 3.6 |
| 120 | VG9B53 | 9 | 17 | 44 | 125 | 10.8 | 81.4 | 3.7 |
| 121 | VG9B41 | 213 | 95 | 29 | 274 | 16.9 | 79.5 | 4.8 |
| 122 | VG9B136 | 192 | 279 | 560 | 1542 | 33.3 | 94.9 | 5.7 |
| 123 | VG9B399 | 157 | 511 | 2400 | 8239 | 36.8 | 96.5 | 7.0 |
| 124 | VG9B369 | 138 | 4897 | 1458 | 2633 | 27.1 | 92.4 | 6.1 |
| 125 | VG9B144 | 215 | 123 | 352 | 1762 | 20.6 | 89.6 | 3.8 |
| 126 | VG9B410 | 158 | 590 | 2119 | 4389 | 36.5 | 98.2 | 12.3 |
| 127 | VG9B386 | 89 | 228 | 846 | 2963 | 29.7 | 97.0 | 8.5 |
| 128 | VG9B125 | 959 | 587 | 1491 | 3755 | 24.8 | 70.8 | 12.4 |
| 129 | VG9B112 | 1255 | 256 | 461 | 1143 | 23.4 | 91.8 | 7.9 |
| 130 | VG9B371 | 4097 | 1555 | 4834 | 7484 | 42.3 | 98.4 | 8.2 |
| 131 | VG9B387 | 135 | 5196 | 746 | 1471 | 24.7 | 89.7 | 5.5 |
| 132 | VG9B47 | 73 | 293 | 383 | 1284 | 30.0 | 96.1 | 6.4 |
| 133 | VG9B379 | 258 | 465 | 710 | 2529 | 29.9 | 94.7 | 7.7 |
| 134 | VG9B447 | 1539 | 197 | 462 | 1303 | 23.3 | 84.6 | 3.4 |
| 135 | VG9B392 | 78 | 191 | 258 | 1548 | 21.2 | 96.5 | 7.6 |
| 136 | VG9B96 | 3808 | 4354 | 2690 | 7663 | 33.1 | 82.5 | 15.1 |
| 137 | VG9B77 | 195 | 864 | 787 | 4675 | 19.4 | 90.3 | 5.1 |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

What is claimed:

1. A method of directing a γδ T cell to a target cell, the method comprising contacting the γδ T cell with a multispecific antibody,
wherein the target cell expresses a second target, and wherein the contacting directs the γδ T cell to the target cell, and
wherein the multispecific antibody is a multispecific TRDV2 antibody that binds a Vδ2 (TRDV2) polypeptide, the multispecific antibody comprising a first binding domain that binds TRDV2, and a second binding domain that binds to the second target; wherein the first binding domain that binds to TRDV2 comprises:
(1) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2037; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2038;
(2) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2071; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2072;
(3) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2105; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2106;
(4) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2139; and (ii) a VL comprising a VL CDR1, a VL CDR2, and a VL CDR3 having an amino acid sequence of a VL CDR1, a VL CDR2, and a VL CDR3, respectively, of a VL having amino acid sequence of SEQ ID NO:2140;
(5) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2173; and (ii) a VL comprising a VL CDR1, a VL CDR2, and a VL CDR3 having an amino acid sequence of a VL CDR1, a VL CDR2, and a VL CDR3, respectively, of a VL having amino acid sequence of SEQ ID NO:2174;
(6) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2207; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2208;
(7) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2241; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2242;
(8) (i) a VH comprising a VH CDR1, a VH CDR2, and a VH CDR3 having an amino acid sequence of a VH CDR1, a VH CDR2, and a VH CDR3, respectively, of a VH having amino acid sequence of SEQ ID NO:2275; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2276;
(9) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2309; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2310;
(10) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2785; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2786;
optionally wherein
(a) the multispecific antibody binds a TRDV2 antigen or a TRDV2 epitope;
(b) the multispecific antibody specifically binds to TRDV2;
(c) the VH CDR1, VH CDR2, VH CDR3, VL CDR1, VL CDR2 and VL CDR3 form a binding site for an antigen or an epitope of the TRDV2; or
(d) the TRDV2 is present on the surface of a T cell.

2. The method of claim 1, wherein the VH CDR1, VH CDR2, VH CDR3, VL CDR1, VL CDR2, and VL CDR3 amino acid sequences are according to
(a) the Kabat numbering system,
(b) the Chothia numbering system,
(c) the AbM numbering system,
(d) the Contact numbering system or
(e) the IMGT numbering system.

3. The method of claim 1, wherein the multispecific antibody:
(a) is a humanized multispecific antibody;
(b) is a multispecific IgG antibody; wherein optionally the IgG antibody is an IgG1, IgG2, IgG3, or IgG4 antibody;
(c) comprises a kappa light chain or a lambda light chain; or
(d) is a monoclonal multispecific antibody.

4. A method of inhibiting growth or proliferation of target cells, the method comprising contacting the target cells with a multispecific antibody, wherein the target cells express a second target on the cell surface, and wherein contacting the target cells with the multispecific antibody inhibits growth or proliferation of the target cells,
wherein the multispecific antibody is a multispecific TRDV2 antibody that binds a V62 (TRDV2) polypeptide, the multispecific antibody comprising a first binding domain that binds TRDV2, and a second binding domain that binds to the second target; wherein the first binding domain that binds to TRDV2 comprises:
(1) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2037; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2038;
(2) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2071; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2072;
(3) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2105; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2106;
(4) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2139; and (ii) a VL comprising a VL CDR1, a VL CDR2, and a VL CDR3 having an amino acid sequence of a VL CDR1, a VL CDR2, and a VL CDR3, respectively, of a VL having amino acid sequence of SEQ ID NO:2140;
(5) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2173; and (ii) a VL comprising a VL CDR1, a VL CDR2, and a VL CDR3 having an amino acid sequence of a VL CDR1, a VL CDR2, and a VL CDR3, respectively, of a VL having amino acid sequence of SEQ ID NO:2174;
(6) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2207; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2208;
(7) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2241; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2242;
(8) (i) a VH comprising a VH CDR1, a VH CDR2, and a VH CDR3 having an amino acid sequence of a VH CDR1, a VH CDR2, and a VH CDR3, respectively, of a VH having amino acid sequence of SEQ ID NO:2275; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2276;
(9) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2309; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2310;
(10) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2785; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2786;
optionally wherein
(a) the multispecific antibody binds a TRDV2 antigen or a TRDV2 epitope;
(b) the multispecific antibody specifically binds to TRDV2;
(c) the VH CDR1, VH CDR2, VH CDR3, VL CDR1, VL CDR2 and VL CDR3 form a binding site for an antigen or an epitope of the TRDV2; or
(d) the TRDV2 is present on the surface of a T cell.

5. The method of claim 4, wherein the VH CDR1, VH CDR2, VH CDR3, VL CDR1, VL CDR2, and VL CDR3 amino acid sequences are according to
(a) the Kabat numbering system,
(b) the Chothia numbering system,
(c) the AbM numbering system,
(d) the Contact numbering system or
(e) the IMGT numbering system.

6. The method of claim 4, wherein the multispecific antibody:
(a) is a humanized multispecific antibody;
(b) is a multispecific IgG antibody; wherein optionally the IgG antibody is an IgG1, IgG2, IgG3, or IgG4 antibody;
(c) comprises a kappa light chain or a lambda light chain; or
(d) is a monoclonal multispecific antibody.

7. The method of claim 4, wherein the target cells are in the presence of a γδ T cell while in contact with the multispecific antibody.

8. A method for eliminating target cells in a subject, comprising administering an effective amount of a multispecific antibody to the subject, wherein the target cells express a second target optionally wherein the subject is a subject in need thereof,
wherein the multispecific antibody is a multispecific TRDV2 antibody that binds a V62 (TRDV2) polypeptide, the multispecific antibody comprising a first binding domain that binds TRDV2, and a second binding domain that binds to the second target; wherein the first binding domain that binds to TRDV2 comprises:
(1) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2037; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2038;
(2) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2071; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2072;
(3) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2105; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2106;
(4) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2139; and (ii) a VL comprising a VL CDR1, a VL CDR2, and a VL CDR3 having an amino acid sequence of a VL CDR1, a VL CDR2, and a VL CDR3, respectively, of a VL having amino acid sequence of SEQ ID NO:2140;
(5) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2173; and (ii) a VL comprising a VL CDR1, a VL CDR2, and a VL CDR3 having an amino acid sequence of a VL CDR1, a VL CDR2, and a VL CDR3, respectively, of a VL having amino acid sequence of SEQ ID NO:2174;
(6) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2207; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2208;
(7) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2241; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2242;
(8) (i) a VH comprising a VH CDR1, a VH CDR2, and a VH CDR3 having an amino acid sequence of a VH CDR1, a VH CDR2, and a VH CDR3, respectively, of a VH having amino acid sequence of SEQ ID NO:2275; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2276;
(9) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2309; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2310;
(10) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2785; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2786;
optionally wherein
(a) the multispecific antibody binds a TRDV2 antigen or a TRDV2 epitope;
(b) the multispecific antibody specifically binds to TRDV2;
(c) the VH CDR1, VH CDR2, VH CDR3, VL CDR1, VL CDR2 and VL CDR3 form a binding site for an antigen or an epitope of the TRDV2; or
(d) the TRDV2 is present on the surface of a T cell.

9. The method of claim 8, wherein the VH CDR1, VH CDR2, VH CDR3, VL CDR1, VL CDR2, and VL CDR3 amino acid sequences are according to
(a) the Kabat numbering system,
(b) the Chothia numbering system,
(c) the AbM numbering system,
(d) the Contact numbering system or
(e) the IMGT numbering system.

10. The method of claim 8, wherein the multispecific antibody:
(a) is a humanized multispecific antibody;
(b) is a multispecific IgG antibody; wherein optionally the IgG antibody is an IgG1, IgG2, IgG3, or IgG4 antibody;
(c) comprises a kappa light chain or a lambda light chain; or
(d) is a monoclonal multispecific antibody.

11. A method for treating a disease caused all or in part by target cells in a subject, comprising administering an effective amount of a multispecific antibody to the subject, wherein the target cells express a second target; optionally wherein the subject is a subject in need thereof,
wherein the multispecific antibody is a multispecific TRDV2 antibody that binds a Vδ2 (TRDV2) polypeptide, the multispecific antibody comprising a first binding domain that binds TRDV2, and a second binding domain that binds to the second target; wherein the first binding domain that binds to TRDV2 comprises:
(1) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2037; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2038;
(2) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2071; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2072;
(3) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2105; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2106;
(4) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2139; and (ii) a VL comprising a VL CDR1, a VL CDR2, and a VL CDR3 having an amino acid sequence of a VL CDR1, a VL CDR2, and a VL CDR3, respectively, of a VL having amino acid sequence of SEQ ID NO:2140;
(5) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2173; and (ii) a VL comprising a VL CDR1, a VL CDR2, and a VL CDR3 having an amino acid sequence of a VL CDR1, a VL CDR2, and a VL CDR3, respectively, of a VL having amino acid sequence of SEQ ID NO:2174;
(6) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2207; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2208;
(7) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2241; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2242;
(8) (i) a VH comprising a VH CDR1, a VH CDR2, and a VH CDR3 having an amino acid sequence of a VH CDR1, a VH CDR2, and a VH CDR3, respectively, of a VH having amino acid sequence of SEQ ID NO:2275; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2276;
(9) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2309; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2310;
(10) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2785; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2786;
optionally wherein
(a) the multispecific antibody binds a TRDV2 antigen or a TRDV2 epitope;
(b) the multispecific antibody specifically binds to TRDV2;
(c) the VH CDR1, VH CDR2, VH CDR3, VL CDR1, VL CDR2 and VL CDR3 form a binding site for an antigen or an epitope of the TRDV2; or
(d) the TRDV2 is present on the surface of a T cell.

12. The method of claim 11, wherein the VH CDR1, VH CDR2, VH CDR3, VL CDR1, VL CDR2, and VL CDR3 amino acid sequences are according to
(a) the Kabat numbering system,
(b) the Chothia numbering system,
(c) the AbM numbering system,
(d) the Contact numbering system or
(e) the IMGT numbering system.

13. The method of claim 11, wherein the multispecific antibody:
(a) is a humanized multispecific antibody;
(b) is a multispecific IgG antibody; wherein optionally the IgG antibody is an IgG1, IgG2, IgG3, or IgG4 antibody;
(c) comprises a kappa light chain or a lambda light chain; or
(d) is a monoclonal multispecific antibody.

14. A method of activating a T cell, wherein:
(a) the T cell expresses TRGV9, and the method comprises contacting the T cell with an antibody;
wherein optionally the contacting results in an increase in CD69, CD25, and/or Granzyme B expression, as compared to a control T cell expressing TRGV9;
(b) the T cell expresses TRDV2, and the method comprises contacting the T cell with the antibody;
wherein optionally the contacting results in an increase in CD69, CD25, and/or Granzyme B expression, as compared to a control T cell expressing TRDV2; or
(c) the T cell expresses TRGDC, and the method comprises contacting the T cell with the antibody;
wherein optionally the contacting results in an increase in CD69, CD25, and/or Granzyme B expression, as compared to a control T cell expressing TRGDC,
wherein the antibody binds a V62 (TRDV2) polypeptide, the antibody comprising:
(1) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2037; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2038;
(2) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2071; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2072;
(3) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2105; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2106;
(4) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2139; and (ii) a VL comprising a VL CDR1, a VL CDR2, and a VL CDR3 having an amino acid sequence of a VL CDR1, a VL CDR2, and a VL CDR3, respectively, of a VL having amino acid sequence of SEQ ID NO:2140;
(5) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2173; and (ii) a VL comprising a VL CDR1, a VL CDR2, and a VL CDR3 having an amino acid sequence of a VL CDR1, a VL CDR2, and a VL CDR3, respectively, of a VL having amino acid sequence of SEQ ID NO:2174;
(6) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2207; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2208;
(7) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2241; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2242;
(8) (i) a VH comprising a VH CDR1, a VH CDR2, and a VH CDR3 having an amino acid sequence of a VH CDR1, a VH CDR2, and a VH CDR3, respectively, of a VH having amino acid sequence of SEQ ID NO:2275; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2276;
(9) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2309; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2310;
(10) (i) a VH comprising a VH CDR1, a VH CDR2, and a VH CDR3 having an amino acid sequence of a VH CDR1, a VH CDR2, and a VH CDR3, respectively, of a VH having the amino acid sequence of SEQ ID NO:2785; and (ii) a VL comprising a VL CDR1, a VL CDR2, and a VL CDR3 having an amino acid sequence of a VL CDR1, a VL CDR2, and a VL CDR3, respectively, of a VL having the amino acid sequence of SEQ ID NO:2786;
optionally wherein
(a) the antibody binds a TRDV2 antigen or a TRDV2 epitope;
(b) the antibody specifically binds to TRDV2;
(c) the VH CDR1, VH CDR2, VH CDR3, VL CDR1, VL CDR2 and VL CDR3 form a binding site for an antigen or an epitope of the TRDV2; or
(d) the TRDV2 is present on the surface of a T cell.

15. The method of claim 14, wherein the VH CDR1, VH CDR2, VH CDR3, VL CDR1, VL CDR2, and VL CDR3 amino acid sequences are according to
(a) the Kabat numbering system,
(b) the Chothia numbering system,
(c) the AbM numbering system,
(d) the Contact numbering system or
(e) the IMGT numbering system.

16. The method of claim 14, wherein the antibody:
(a) is a humanized antibody;
(b) is an IgG antibody; wherein optionally the IgG antibody is an IgG1, IgG2, IgG3, or IgG4 antibody;
(c) comprises a kappa light chain or a lambda light chain; or
(d) is a monoclonal antibody.

17. The antibody of claim 14, wherein the antibody is multivalent; wherein optionally
(a) the antibody is capable of binding at least three antigens;
(b) the antibody is capable of binding at least four antigens; or
(c) the antibody is capable of binding at least five antigens.

18. The antibody of claim 14, wherein the antibody is a multispecific antibody, wherein optionally the multispecific antibody is a bispecific antibody, a trispecific antibody, or a quadraspecific antibody.
